SEQUENCE LISTING (363 sequences)

SEQ ID NO: 1 - DNA, 3626 bp, Homo sapiens
cgggcacgggtcggccgcaatccagcctgggcggagccggagttgcgagccgctgcctagaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccacaggctgctggccccaggatacatagaaacccactacggcccagatgggcagccagtggtgctggcccccaaccacacggtgagatgcttccatgggctctgggatgcaccgccagaggatcattgccactaccaagggcgagtaaggggcttccccgactcctgggtagtcctctgcacctgctctgggatgagtggcctgatcaccctcagcaggaatgccagctattatctgcgtccctggccaccccggggctccaaggacttctcaacccacgagatctttcggatggagcagctgctcacctggaaaggaacctgtggccacagggatcctgggaacaaagcgggcatgaccagccttcctggtggtccccagagcaggggcaggcgagaagcgcgcaggacccggaagtacctggaactgtacattgtggcagaccacaccctgttcttgactcggcaccgaaacttgaaccacaccaaacagcgtctcctggaagtcgccaactacgtggaccagcttctcaggactctggacattcaggtggcgctgaccggcctggaggtgtggaccgagcgggaccgcagccgcgtcacgcaggacgccaacgccacgctctgggccttcctgcagtggcgccgggggctgtgggcgcagcggccccacgactccgcgcagctgctcacgggccgcgccttccagggcgccacagtgggcctggcgcccgtcgagggcatgtgccgcgccgagagctcgggaggcgtgagcacggaccactcggagctccccatcggcgccgcagccaccatggcccatgagatcggccacagcctcggcctcagccacgaccccgacggctgctgcgtggaggctgcggccgagtccggaggctgcgtcatggctgcggccaccgggcacccgtttccgcgcgtgttcagcgcctgcagccgccgccagctgcgcgccttcttccgcaaggggggcggcgcttgcctctccaatgccccggaccccggactcccggtgccgccggcgctctgcgggaacggcttcgtggaagcgggcgaggagtgtgactgcggccctggccaggagtgccgcgacctctgctgctttgctcacaactgctcgctgcgcccgggggcccagtgcgcccacggggactgctgcgtgcgctgcctgctgaagccggctggagcgctgtgccgccaggccatgggtgactgtgacctccctgagttttgcacgggcacctcctcccactgtcccccagacgtttacctactggacggctcaccctgtgccaggggcagtggctactgctgggatggcgcatgtcccacgctggagcagcagtgccagcagctctgggggcctggctcccacccagctcccgaggcctgtttccaggtggtgaactctgcgggagatgctcatggaaactgcggccaggacagcgagggccacttcctgccctgtgcagggagggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagcagatcaagtccagatgccaagatcctgcctctggtgagaggtagctcctaaaatgaacagatttaaagacaggtggccactgacagccactccaggaacttgaactgcaggggcagagccagtgaatcaccggacctccagcacctgcaggcagcttggaagtttcttccccgagtggagcttcgacccacccactccaggaacccagagccacattagaagttcctgagggctggagaacactgctgggcacactctccagctcaataaaccatcagtcccagaagcaaaggtcacacagcccctgacctccctcaccagtggaggctgggtagtgctggccatcccaaaagggctctgtcctgggagtctggtgtgtctcctacatgcaatttccacggacccagctctgtggagggcatgactgctggccagaagctagtggtcctggggccctatggttcgactgagtccacactcccctgcagcctggctggcctctgcaaacaaacataattttggggaccttccttcctgtttcttcccaccctgtcttctcccctaggtggttcctgagcccccacccccaatcccagtgctacacctgaggttctggagctcagaatctgacagcctctcccccattctgtgtgtgtcggggggacagagggaaccatttaagaaaagataccaaagtagaagtcaaaagaaagacatgttggctataggcgtggtggctcatgcctataatcccagcactttgggaagccggggtaggaggatcaccagaggccagcaggtccacaccagcctgggcaacacagcaagacaccgcatctacagaaaaattttaaaattagctgggcgtggtggtgtgtacctgtaggcctagctgctcaggaggctgaagcaggaggatcacttgagcctgagttcaacactgcagtgagctatggtggcaccactgcactccagcctgggtgacagagcaagaccctgtctctaaaataaattttaaaaagacataaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 227 bp, Homo sapiens
accgggcacgggtcggccgcaatccagcctgggcggagccggagttgcgagccgctgcctagaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggac

SEQ ID NO: 3 - DNA, 3509 bp, Homo sapiens
cagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccacaggctgctggccccaggatacatagaaacccactacggcccagatgggcagccagtggtgctggcccccaaccacacggatcattgccactaccaagggcgagtaaggggcttccccgactcctgggtagtcctctgcacctgctctgggatgagtggcctgatcaccctcagcaggaatgccagctattatctgcgtccctggccaccccggggctccaaggacttctcaacccacgagatctttcggatggagcagctgctcacctggaaaggaacctgtggccacagggatcctgggaacaaagcgggcatgaccagccttcctggtggtccccagagcaggggcaggcgagaagcgcgcaggacccggaagtacctggaactgtacattgtggcagaccacaccctgttcttgactcggcaccgaaacttgaaccacaccaaacagcgtctcctggaagtcgccaactacgtggaccagcttctcaggactctggacattcaggtggcgctgaccggcctggaggtgtggaccgagcgggaccgcagccgcgtcacgcaggacgccaacgccacgctctgggccttcctgcagtggcgccgggggctgtgggcgcagcggccccacgactccgcgcagctgctcacgggccgcgccttccagggcgccacagtgggcctggcgcccgtcgagggcatgtgccgcgccgagagctcgggaggcgtgagcacggaccactcggagctccccatcggcgccgcagccaccatggcccatgagatcggccacagcctcggcctcagccacgaccccgacggctgctgcgtggaggctgcggccgagtccggaggctgcgtcatggctgcggccaccgggcacccgtttccgcgcgtgttcagcgcctgcagccgccgccagctgcgcgccttcttccgcaaggggggcggcgcttgcctctccaatgccccggaccccggactcccggtgccgccggcgctctgcgggaacggcttcgtggaagcgggcgaggagtgtgactgcggccctggccaggagtgccgcgacctctgctgctttgctcacaactgctcgctgcgcccgggggcccagtgcgcccacggggactgctgcgtgcgctgcctgctgaagccggctggagcgctgtgccgccaggccatgggtgactgtgacctccctgagttttgcacgggcacctcctcccactgtcccccagacgtttacctactggacggctcaccctgtgccaggggcagtggctactgctgggatggcgcatgtcccacgctggagcagcagtgccagcagctctgggggcctggctcccacccagctcccgaggcctgtttccaggtggtgaactctgcgggagatgctcatggaaactgcggccaggacagcgagggccacttcctgccctgtgcagggagggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagatcaagtccagatgccaagatcctgcctctggtgagaggtagctcctaaaatgaacagatttaaagacaggtggccactgacagccactccaggaacttgaactgcaggggcagagccagtgaatcaccggacctccagcacctgcaggcagcttggaagtttcttccccgagtggagcttcgacccacccactccaggaacccagagccacattagaagttcctgagggctggagaacactgctgggcacactctccagctcaataaaccatcagtcccagaagcaaaggtcacacagcccctgacctccctcaccagtggaggctgggtagtgctggccatcccaaaagggctctgtcctgggagtctggtgtgtctcctacatgcaatttccacggacccagctctgtggagggcatgactgctggccagaagctagtggtcctggggccctatggttcgactgagtccacactcccctgcagcctggctggcctctgcaaacaaacataattttggggaccttccttcctgtttcttcccaccctgtcttctcccctaggtggttcctgagcccccacccccaatcccagtgctacacctgaggttctggagctcagaatctgacagcctctcccccattctgtgtgtgtcggggggacagagggaaccatttaagaaaagataccaaagtagaagtcaaaagaaagacatgttggctataggcgtggtggctcatgcctataatcccagcactttgggaagccggggtaggaggatcaccagaggccagcaggtccacaccagcctgggcaacacagcaagacaccgcatctacagaaaaattttaaaattagctgggcgtggtggtgtgtacctgtaggcctagctgctcaggaggctgaagcaggaggatcacttgagcctgagttcaacactgcagtgagctatggtggcaccactgcactccagcctgggtgacagagcaagaccctgtctctaaaataaattttaaaaagacataaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 826 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHRLLAPGYIETHYGPDGQPVVLAPNHTVRCFHGLWDAPPEDHCHYQGRVRGFPDSWVVLCTCSGMSGLITLSRNASYYLRPWPPRGSKDFSTHEIFRMEQLLTWKGTCGHRDPGNKAGMTSLPGGPQSRGRREARRTRKYLELYIVADHTLFLTRHRNLNHTKQRLLEVANYVDQLLRTLDIQVALTGLEVWTERDRSRVTQDANATLWAFLQWRRGLWAQRPHDSAQLLTGRAFQGATVGLAPVEGMCRAESSGGVSTDHSELPIGAAATMAHEIGHSLGLSHDPDGCCVEAAAESGGCVMAAATGHPFPRVFSACSRRQLRAFFRKGGGACLSNAPDPGLPVPPALCGNGFVEAGEECDCGPGQECRDLCCFAHNCSLRPGAQCAHGDCCVRCLLKPAGALCRQAMGDCDLPEFCTGTSSHCPPDVYLLDGSPCARGSGYCWDGACPTLEQQCQQLWGPGSHPAPEACFQVVNSAGDAHGNCGQDSEGHFLPCAGRDALCGKLQCQGGKPSLLAPHMVPVDSTVHLDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFLLAMLLSVLLPLLPGAGLAWCCYRLPGAHLQRCSWGCRRDPACSGPKDGPHRDHPLGGVHPMELGPTATGQPWPLDPENSHEPSSHPEKPLPAVSPDPQADQVQMPRSCLW

SEQ ID NO: 5 - DNA, 207433 bp, Homo sapiens
gctctaataaatttgcggccgctaatacgactcactatagggagaggatccgcggaattcccccatgtgccatgtccacgagatggttgaacagatgagacaacatggttgtgcagcttctctctttttttttttttcagacagagtctcactctgtagcccaggctggagtgcagtggcgcaatttcagctcactgcaacctccgcctcccaggtcctgattcaagcagttctcctgcctcagcctcctgagtagctgggattacaggaacgcgccactatgcccagctaatttttctaggtttagtagagacggggtttcaccatgttgaccaggctggtcttgaattcctgaccttgtgatccgcccgcctcggcctcccgaagtgctgagattacaggcatgagccaccgcatccggccatgcagcttctcttttctagagttaacaggagatgcggcaggtctgtagtagcccagaaattctcagacttgcactcttgaatttacacgcatgtacaaaatgcagctcagcctaatgcctcctgttcagttctgtggtgctgcctacttcctgtgcccaaattagccttgcgtttataactaggaggaatgacttggtgtcctacaatattttggctcctgggcttctttggtgttcattctattactagttgattttttttttcttggaaagaaaagtattcaaagagccagaatttactttcattatatcttacagttgttaaattaacctatccttgttttcactttctgtgtacttctttctttggggtacaaaacagtgtttctttagattgtctatttctaaatattattttaaccagaacataccaaatatattttcctgccagtttcattcttcattttacttcttaaccattgttacgattttttttttaacttcggtctcagattttgctcaattgaagttgtcccagttcatgagattttgttttctttgcagctcttgaccatgacagatgtgaccagcacacagattgttacagctgcacagccaacaccaatgactgccactggtgcaatgaccattgtgtccccaggaaccacagctgctcagaaggccaggtcagaggctgtttcttaaagatttcagaaaaatcccaatttgtcataggtttagcttttatagtgtatatggtataaataatggcccagagttacttttcaaatgggtttctatttggattttattatccctgaggttttcctttaggaagagatgttctgtatatttcaagggtcctactagccctgggaatttgtgtattgtgttttagaagaaggtaggactgttgtccctgggcatggctgtgactaagcactggatccttggtttgaggcatctgatagtgacctcactttaccagtaccaggttttgataaagttagggtttgagagtgagtcaggtgtacagcggcccatattgaactgtggaaatgacagactttgcaaaatctccttgttttatatcttggtttggcataatctcactgctcatcatatatagatttttaaatttaagttataagatgacccaggcttttatagtttttgacagcttacaagacttttttttttgtcagtctcatacagttcataaaatagaaaactttcagacttttgaagtgagcatttgaaaagcaccaagttcaacattcccattttacagatgagcaggttgaggcatggtgtttaaaagagtgggctgcttccttgccagttaaaagcatgtccttgagcagaacattccattgcagtgttccccactgcagagctactgcaacttatctatctatctgtctgtctgtctgtctatctatctatctatctatctatctatctatctatctatctgtctatctatttatttgcagtttgcttcctcaattagattttgttgtctataatatcctgttggtcctcaccataagttctctctgtggatacaaaagaaaagcttttgtccactgtgttctagatctccatttttaggtatgagaattgccccaaggataaccccatgtactactgtaacaagaagaccagctgcaggagctgtgccctggaccagaactgccagtgggagccccggaatcaggagtgcattgccctgcccggtaggccttgcagggtcatcttggtgtgtgtgggtccattacttcagcctgcttcccccaacactgtgcagcctaagttgaacctagcagaggggaagagctaattctgtccattcatcccccacacgagtattatgggcttttttgtttttaactaaaatacagttcttaagtatttgttcctactgtcctttgaaataaagtgaaacatcctttgctgctctgtagaattgagtgacagcttggttctgtatcactgagcctgctccttgtctctttcccatccacctttgataacctgggactagaccatgatgtctcagcagtcagtctgctgagcactttatggagagtacttcttattaaccactgggatttaattgttggcacctgctaatgggccttctctgagaaggagaggatagatacttctgtcagcagcaccttttaggggtgatctccagccctgaaaacctcaatatatcctgcttctgaggttcaggatgatgaactcagggcctgagaccagccagccatgtgatatatttggaccagggtggtccagaaaggggaacttccttgtctgtgcacaacatctgagcttgttcagggaagttggtttggaccaggccctttttgaatgtcccttggaagtttttgattcagcttgcagagtgggactcttttctatatctcagtgtgtctcaaattttaaggtacaggaaaagacctgggaatcttgcgaaaggcatattctggtgcaagtgtggggtggggcctagagtgtgcattttatctagtcgactgctacactgtgaggagcaagtctttgtctgttcttaaatgatctctttcccatggtacctttcttttatctcagtgactgttactgttaatgaacattgttgatgtctccaaagtactttggttctggtgaagttgctttgttctttcattgtcttccaggaagcattcatagcttctgtgcagtaccttgtgtgggttcaggatgatcacaggtagcagattacaagcttgtcttgtatgctatagccatatcacttgggttgtttctcaagaaggaccttctcaccttgcttttgggatgctttgtacacttgattgtaccttccacctgatgatatgaaaacagtgcagcttttggagactatagatttgttaattccttgattcatttccattcttgcagtttttaccccagccctccaatatgcatattcatttgtctgctcttcacttaggattttagttttctaattgttcttcagaaggaagtgtaccagtctaatattggcaccaaactggtgttttcatctaagacataggataagtgacctcagaatatgctttttaggatccgggagatatcaccagtaaacattttaaaattcttgtattctgcatttggtccttaataatgtgtcagaggctcccacatcctaatgaagtacctagaatttaaattagaaaggccatttcggtattcagtaatttgaactcataatacagtagttttgtctgatttctaaaattcttttctttctcttttccccttaatgaaagaaaatatctgtggcattggctggcatttggttggaaactcatgtttgaaaattactactgccaaggagaattatgacaatgctaaattgttctgtaggaaccacaatgcccttttggcttctcttacaacccagaagaaggtagaatttgtccttaagcagctgcgaataatgcagtcatctcagagcatggtgagttaaaatcctcaaaacttaagtttctggttatccacctttctaccaagggcatgactgcagcttgcatgtggaaggctgtggatatgtgtaacgtgcttggcaagaaggggagtgctggtgaacgcagcctgagggactgtgggtttgtgctgtcagagtctcttcctcttaaaatttttaatactttgtatatataagatctatgaataattatatgggggatgaattgtaacatgtatatgtgtacataatctggtgacatcagtagattatttcatacctgttttacctctggattctgctaggggagaaagagaggtcactgataattagctaggttggattaagccacctgagttccttggagttaaggtattataatagtgcataagactgtataattaccactaagaagtgtacatctcagctggatgtggtggctcacacctgtaatcccagcactttgggaggctgaggtgggtggatcgcctgaggtcaggagttcaagaccagcctgaccaatatagtaaaaccccgtctctactaaaaatacaaaaattagccaggcatggtggtgtgcgcctgtaatcccagctactcaggaggccgaggcaggagaattgcttgaacccaggaggtggaggtggcagtgagctgagattgcgccaccgcactccagcctgggtgacagagcgatactccgtctcaaaacaaaaaaagaacagcaaaaaaaagaaatgtacatctccttgggtctttcatagcccagccatctcaaaagaagagagcaccttcttgtcaagagtttctaagcccagaaaggctcaagttctctgcttgtccacccagtgctctcagggggcttatagtcaatattccatgatcacattttgtcatttttagtctggagtcataaattgtgatcctaccgtcagttaagtagactcatagaacaaagctctttcagcagtttcagctgtggtacagaaacgtttagtggaaatgttcttaccaagcagggaggagttgagggcaacacttccttgggtacagcctccttcatgtgaaggtatggaaatgtggcctgggtctcggctgcctgtggcctctgtgtaccacctatagggccattctgagactggtaggaggtgccctgtatttagttttctccaattagtcccttttttcagtgcaacttagatggggtatggacactcaaacattggtgacatattcttagtgtgtttacctcaggctactgtgaccacatttggtatttcataatattttgatagctttttcagatttcagaatctccattggtgactgtctctgttgtttctctttccatgtccaaatgtgggtctcttccagcattccatccttggctggcagctgacctttcccatagttattcactccctcagaaaatggatggcacccagctttgcttattaccctggtgtctctaacaatgactctcgagtccacggaagttaaaagggttcaaccaggtggccacagatatacttctggtaccctttctctcttccttaggttttctaactctaaacgtttctgggtattctaatctgctgtggccacgtttatgaaacagaaattcacagtcttaatgataagactgacagatgagagacaactgaagtgtaatgtccttccacagctatacctctagatgtagcccagttagtagagcccagatttttatggaaaaacaagaaaggacacctagcctaacccttaggacagaggctggctggtagaaagctggaaggaggtgacccctgcaggtgagaaggagtgaattaggatgtcgaagacggaagggctttctgtgatttaattagtgcccccatctgtgagatgtagagggagatgattaagggagtggctctttgagtgagctgcaggtaagtttgcatggttggctgcaggctggatgggaggggatttttatagttgagcctcaggaaggaaccaaggccagaccctgcgaggccatggctacaatagtaatggatttgaattgtatcgtgaaggcaaagaaattattgaagggcctttaaaaagtatttttaattgtctttctttcttttggatctgtttatttttaggtcttttagtaggcatgtgtattttttcctctcaaaaatggaaataggctgggtgtggtggctcctgcctgtaatcccagcattttgggaggccagtgagggaggattgcttgagcccaggagttcaagactagcctgggcaacacagggagacctcgtctctacaaaggaagtttttttaaagaattagccgggcatgatggtggcacatacttgtagttccggctacttgggaggctgaggcaagagggttgcttgagcccaggggtttgaggctgtagtgagccatgatcatgtccctgcactccagcatgggcaacagagcgagaccctgtctcaaaagaaagcaaagggagggaaatacagtatatcttttgttttataactaccaaaattaggaatacttaccatttcttggctaaactttatattttgatttttaaaacttgttaaaaattgcaatgagaaggaaatttcaggagagcagaagacagactgtcccaggtgtcactgtcctattattccctataaaatccagtgccaggatggatgaatggataaagcaaatgtggtataagttgaatatcccttatctaaaatgctttggacgaggagtgtttcgaattttagaatatttgcattatactaaccagttaagcatccctaatctggaaatccaaaatgctctagtgaacattttccttcagcatccatcatgttggcactcaaaaagttttagattttggagtgttttggatttcagattagggatactcagcctgtgtttgggggtagccatctcttcatatagacatttcagaacttaaatattgctttgctataatttctgtgaatttttgatatattatcttctctgagctacatttttatcctttataaaatggccatattgaagtgatgatctatcctaatctaccatggctgagtcaagggataaagaggttttcctgtgtctgtggggtatacttaacttggtggtttttatctagaagcttgttttggtcaagatgttggttatattcaggccaggcatggtggctcatgcctataatctcaacattttgggaggccaaggtgggaggatcacttgagctcaggagtttgaaactagccagggcaacatggcaagactccatctccaaaatttaaattaaaaaaagatactatctgtattcatagttgtgtctcttttgcctttagtccaagctcaccttaaccccatgggtcggccttcggaagatcaatgtgtcctactggtgctgggaagatatgtccccatttacaaatagtttactacagtggatgccgtctgagcccagtgatgctggattctgtggaattttatcagaacccagtactcggggactgaaggctgcaacctgcatcaacccactcaatggtagtgtctgtgaaaggcctggtaagttcacaggtgaattaggtggtattcagagtttattgtgagagaaaccataggaggcatagttcattgctgagatgtgtgaagtagtcatgaaaacagatgaagtattgatttcaagcatgcaaagaagagtataacccagatttcagaagcagaaggaaatattctgggaccctgaatagttttaattataagcaaaactaaaaataactaacactactcgaagaaactgatattctaattaacaatgagattgataggtttattgaccagaaaaagtattgagaattgctctgaaaagcaaatttattggtgttggcagagaaatgctgtggaagaagaaacaaaaaaggaaaataaaaccaaagaaaagattagtaaagcaagcaagtgactgcagggacagtgttcagaaaggtagtgtcaacagggagaaaaatgatgagagcagttctgtaagcgaggacagaagcaacccagaactacgcgagagctgcaggagagtactgagcagacagacactcggagtagcttcccagctttcagtctctctgggctacattttggctaactaaaggcaggcccaggggtagctgctgcagcatgaagacaactcaaaataagcctcctcctcctgcagagggactcacaagcagtcggtggaattgtgggttattttgggaggctggactccatttgcattgtgtccaattttgggaaagtaatttgtctgaggaattacagaggtataggagagaattcatagcattgtagatttctaaatattgatttctaaacttctctaggggagctgcccagcagcctttcagaattctgaatcctttgttgaacttaatgaatgccgtagaccttcttccctccaaaattgcaaacatgagattttacatattcaagtggattgtaaaacccctaaagttcatcctgggtccccaaattctaaggggcttacagccctacttttatggaaagattcactgtttcctcatctttgtccttaatgctctttgaaaagaaaatttattttttccacaagtgtgtaaatacacttgactaaacagtacttgatgacttttattgttttctattttctctcatttaattgatttcatggcacattaatgggtcatcacccacatttgaaaagtcttggctgggcacagtggctcacgcctgtaatcccagcactttaggaggccaagacgggtggatcacaaggtcaagatatcaagaccatcctggctaacatagtgaaaccccatctctactaaaaatacaaaaaattagccagacgtgatggcgcacccctgtagtcccagctacttgggaggctgaggcaggagaatcacttgaaccggggaggcggaggttgcagtgagccgaaattgtgccactgcactctagcctggagacagagtgagactccgtctcaaaaaaaaaaaagaaaagaaagaaagaaaagtctttgaccttagcggacatggtgggagctctaagtgtctctcttgggtttcattcccagcaaaccacagtgctaagcagtgccggacaccatgtgccttgaggacagcatgtggagattgcaccagcggcagctctgagtgcatgtggtgcagcaacatgaagcagtgtgtggactccaatgcctacgtggcctccttcccttttggccagtgtatggaatggtatacgatgagcacctgcccccgtaagtgaaaaagggagccctaggcacttatgcatgccctctgtataggcaacaactcagccatgaggctgtgctgtcagcctctgaacattttagaaacaagactggacatgacctctgctcaaacctgaccagagactgccatcgagaccttgctgcctattgagaaccttcatacagaatcaggcacattgacagtaaataaatgtaagatagatcacagagtacagaaataacttgtccaacttcagtgtcatattgctcaatccatgtaatatctccatatctgaatttcctaatttgtaaagtaaatgctttccaatagataatctctaaggtcccttttgccttcaacatcctgggattgagagaggagggaagggtcatctctgttatgtattgggcaaaatactgggctctttacattcattatctcttttaaataatcaagacagaataatatttttgactcaagccagttgaatagtctgttaaaaaaaaagtaaatacagtgaattcagatctacctgtgatagtcaattgcaactttttttttttaatagctgaaaattgttcaggctactgtacctgtagtcattgcttggagcaaccaggctgtggctggtgtactgatcccagcaatactggcaaagggaaatgcatagagggttcctataaaggaccagtgaagatgccttcgcaagcccctacaggaaatttctatccacagcccctgctcaattccagcatgtgtctagaggacagcagatacaactggtctttcattcactgtccaggtaagatgccttgcatatccaaattcaagtgtttcactactgatttatgaagaataaaaccttgaaagctacgttgtgtatatgtaactccctgccctcagcccctttccttcctcctaatggttggtacaagaaggaatagaccagaagctggtccaaggcctgacctggacctgctgagagtggtggtgggttcctaagaaaccaattctaagaaattggcctttgattcagacttgaagtgaccactcagcaatgtgtctgtgggtttctagaacagttgggagaggctgggctggtgcaaagactcctcagagattagcagtcaagaacttctctaagagcctgccattgacaacagggctgtttgtgaggactttgtaagggaaagtccactgtaaacaaagctaaaagggcagagacagactgggagaaaatacctgcactgcatgtaacaactgatgatcatccagaatatgtaaactcccaccctccagggaaaaaataagaagctaaatgtgaacccaacagaaaaagtggttattggagataaagaagccattctcagaaaaggaaatagaacaggaaaatgtaaactaacaccaggaaccaattttttgtctactaaactggatcaaattttccgtgttcccttttttccatactaagatatgggggacctgcaattctatttttaatctgctaggagttaactttttaacgaaatatttaaatctctgctttttcatgaatatcacgaatatatctggtaaaatgacaacccagagaatgggagaaaatatttgcaaagtatatatctaataagaatccaatgtccagaacacgtaactcttaaaactcaacaatagaaagacaacccaattaagaaatggataaaggatttaaatagacatatgtccagagaagaaatacaaatggccaataagcacatgaaaagatactcaatatcattcatcattaccaagaaatgcaagtcaaagccacaatgagataccactttacacccactgagatggctgtaatcaataaaacaggtaataacaagtattggaaggatatgtagaaattggaactctcatgcaggttggcaggtcctcaaaaagttaaatatagagttatcatacggcccagcagttttactcctaggtacatacccaagaaaattgaaaacatatgttacccaaaaacttgtatataaatgcttatgcttatagcatcattcttcatagcatcatgctcaaagcaacattattcataataagcaaaagtgaaaacaagccaaatacctgttagctggcaaatggatgaacaaagtgttgtatattcatacagtgtaatgttatttggcaataaaaaggaatgaagtactgacatattttctgacatggattaccctaaaaaaacatgctatgtaaaagaagccaggtgcaaaagattatgcgttgcatgatgccatttatatgaaatgtccaaagacagaaagtagatgtttagtggtttcctagggctgggcatggtaatgaagaataataggcatgaggttttctagagtagctgcagcattattttctgacatggattaccctaaaaaaaatgctatgtgaaagaagccaggtacaaaagattatgcattgcatgatgccatttatatgaaatgtccaaaggcagaaagtagatgtttagtggtttcctagggctggggatggaaacaaggaataataggcatgaggttttctagagtagctgcagtattattttctgacatgaattaccctaaaaaaacatgctatgtaaaagaagccaggtacaaaagattatgcattgcatgatgccatttatatgaaatgtccaaaggcagaaagtagatgtttagtggtttcctagggctggggatggaaacaaggaataataggcatgagattttgtagagtagctgcaccattttatgctctcaccaacatcatatgaaggttcatatctttccacatccttgccaatacttgttactatctttttaatgaaaactgttctagtggatgagaaatggcatctcactgttgttttcatttgtattttcctgataactaatgaaattgaacatcaacttcattagttagccttttgtatatcttctttggagaaatatttagtcaaattctttgcccatttttcagttatgttgtcttttttattattgagttgtaagagttctttatgaattctgaagtcccttattggatatattatttgcaaatactttctcccattatatgggtttcttttcactttcttaatatgccttttgaaatcaaaagttttcagttttgattaagttccatgtatcagtgttttattttatcccatgtgctttttggtattgtatctagaaaatcagtgcccaagtaacccaggatcacatagatttgctcctaagttttctttgaaaagtttcatagatttgtgtgttacattaggtccttgatccattttgagttaatttttgtgtatggtgtgaggtaggggtccaaactctctttttgcctgtggatgtggtcctgcaccatttgttgaaaagatttttttttttttttaaccattgaattttcatggcacatttgttgcagatcagttgactgtgaatctaggaacttttttctaagctctcatttttgtgcagttgaccaatgtttctccttttgccaatattacactgttttgattactgtggctttgtataagttttaaaattgggaaggctaagtcctccacctttgttcttattttgcaagactgttacagctattctgggtttcttgcatgttcatattaattttaggatgagcttgccattttctgcaacaacaaaaagccaactggtttgataaggtttgcattgaatctactgaccaatttggggtgtattgcctgtttgtttgtttgtttgtttgtttgtttgtttgttgagagagggtctcactctgtcacccaggctggagcgcagtggtgctatcttggctcactgcagcctccgctttcccaggctcaagtgattctccagcctcagcctctcgagtaactggtactacaggcatgagctaccaacacccagctaatttttatatttttttagagacagggtttcgccatgttgcccaggctggtcttgaactcctgagctcaaagcaatcctcctggcctttgcctccctagttctgggattacaggtgtgagccaccacgcctgggccccttgccattttaacaagggttaatcttctagcctgtgaacctggaatgtcaatttattaccctctttactttcttttggcattgttttatagttttcagtgtacatcattgtcttgttcctttttttaggggaaaataattcagtctttattaaatatgatatttgtttagttttttttatacatgccctttattgggttgagaaagtttccttctattctagttttttgagggttttgtaatgaatgagtgctagattttgtcaaacactttttctgtgtctacttagatgatcacgaagttttgttctttattaatactatgtattacattaattaagaatgttaaaccagttttgcattcctgggataaagcccatttagtcatgatgtatacttttttatcatgatgtatatttttaatctgctgttggattctatttgctagttggggattttgtgtgtacattcatgagggggatataagtttgtggttttcttcttttgtgattactatctggttttcatagcagggtagtactggcctcctagagtaagttggaacgtattcctcctcctctatttactggaagagttcgtcaaggattagcattaattcttctttagatgttgattgaattcaccagtgaaaccaaatgggcctggcctttttctttacaggaaaatttttaattattaattcaatctgtttgttatagatctattcagattttctttttctgagtgagtcagttttggtaatctgtttttctaggaatttgtctattctcagtaatcaaatttgttaacatacagttcgtagtattttcttttgtttttttttttttttttttgagacagagtctcactctttcacccagtctggagtgcagtgacgtgatctctgcttactgcaacctccgcctccagggttcaagtgattctcctgcctcagccccccaagtagctgggactacaggcgcacgccaccactcctagctaattttttattttgttttgttttgctttttttagtagagacagggttttaccatgtagaccaggatggtctcgatctcctgacctcgtgatctgcctgccttggcctcccaaagtgctgggattacaggcgtgagccaccacgcccggccagttcatagtattttcttgcaggttgctctttagcaatatgtagtctcctgtttaacctgttaacgatttttttttctgtctaatttggttttattattcttttacattttgcaagggctctaaatattgtgagggtttttttttttaagagcttgctctcttatattgtggatgcaataatttaagccttattgaatgtactaaaattcgtatttttcattctctcatatttcttgcattaacccttccttcaggatttgttgctgtgcgtgtttatcctggtggtattcggagattgagagttttcttatactggttaatcctcaaggttaatttgtattaataactaaatgtgtagatctgtcaatattggtcagtggttgggtgtgctgagctgttgtgaaagtgtggtatgatgtcctggaaaagctagcaagccagcctcaggacactccagctctggtccacttcgactgttgttgactggtgtgcgcatctccctcattggaagaaggaaggagcaatggacctgggtggctgggtgcaattagcatgttcatgggtgtggcaagtgcctcccagccttgggtggtagggccacctaaagatagctggcacattgccttgagttttctttcattcttgcttgcttgcttgcttttatttttgagatggagtttcactcttgttgcccaggctggagtgcaatggtgctttcttggctcactgcaacctctgcctcccgggttcaagcggttctcctgcctcagcctcccaagtagctgggattacaggcacctgccaccacctctggctaattttttgtatttttagtagagacagggtttcactgtgttggccaggctgatctcgaactcctgacctcaggtgatccacctaccttggcctcccaaagtgatgggattacaggcatgagccaccacacccagctgagttttctttcaaacactcaaatactaacaggtgcataaacagaacaagtacaaggctatggaaggttgccaagatggtgaaaaactgcatcacactgatcaaccaaggcagatctgaagaatcagattttgcttacagtgcaggttgtgcactatgtaaatgtttcccccatcctattcccccctagagttctgcagtgcacagcctgtgcagctgtcctgagcagtcctcagaggtccaagcttcatatacctttctagttgtaaagccctatatccattaggagtcttagtaatcccacttgtggaaatgtttatccgtaaagatggttatttttctaataatgaaaaatgtaaagcaaactaaatattagaatataagggaatggttaaataactgttaagattcatcaaaatcattctaccatttgaagttttgtttataattgatgaaataaggtggtaaaatagttgtgatctaagttaagaaaggtgaatattaatatgaacataattgtgcaaattatgttcttctggtatagaaaaataccagaagaaaatatatcaaatgtgaacagtggtctctgagtggttggattgtatatgatctttccccatccttctattttttaagtatactgtcatgagcatacgttattttttagtgaataacaacaaaaaatattttttaatatcactatgggctttcaccttgctgtgatacattttcaattttcctggactagctcttctttgatattttttatcttatttctcaaattagtttttagtgggccagttttggtgatttatcaaagataaattaactacagagatagttgcagataaatcttattgaacttaactacatgcagttcttccatggtacagaccccttgaaactctttttcttgcagcttgccaatgcaacggccacagtaaatgcatcaatcagagcatctgtgagaagtgtgagaacctgaccacaggcaagcactgcgagacctgcatatctggcttctacggtgatcccaccaatggagggaaatgtcagcgtaagtcaaattggtcaggtttactcatggcaaatcggtgtggaacacagcacttctatttgacttgaatatctaggaggaaaaaagccactttgttttggataccacatttcttattaaatcatactggtcagaagctcagctgagctggaagaaagaaccaaggtttcagtgtagctggttaaagcaacaagccaaaaatgttagagtcaagcctttgaccacattctgcagtggtataagcaagggcccaaaaccagagaaagtgccaacttcccctgtttcctttcctcccattgaacagggcacaggtcaatagtcagagggatcagctcagacatgggagtcttggggctcatctcactgaggcccggacaaaaggttccagcagtatatggacactaaggttgtgggaagggcaaggtgtaagggctaggactgggaaagtcctgggttggagtcagagttaggaaagtgtcttctgaattccccctcctccccccatcaggaggccttggtgcctgaagaagacctcagcccaaggcctcagatagggagcctgggaataaaggctccagggctaggaatgtgaacatgtgaaggagcgtgacccgtgaggtagacctgagtccttaactgcacctctctcagagccccacacttaggcccacctcacaggtcatgctctgccaggtaaagcctgtggcattgcctgtggtcaggccctgaaaaatcacacacgggctttttaaccaggagccagactcctcaatacttatagtgtattttaaaggtttaaatagtcccgtgttggctgggtgtagtggctcacgcctgtaatcccagcactttgggagggccaaggcaggtggatcacttgaggttaaggagtttgagaccagcctgaccaacatggtgaaaccccatctctactaaaaatagaaaaattagctgggcatggtggcgggggcctgtaatccaagccactcaggaggctgaggcatgagaattgcttgaacctgggaggcggaggttgcagtgagctaagatcacgccactgcactccagcctgggcaacagagtgagactcagtctcaaaaaaaaaaaaaaagaaaaaaaaaaaagtcctgtgtttccccatttatgtcaaatagaagcctgcaagcaataggacattttattatgagaacaaaaattatgacaagtgatattaatgaactgctttttttattctagtttccagccaaaaaacataatgagtactatagtcgaaagacttttcaaagttctgagcaggaaaagtaaacaacatagggaaatctctacctactccccagatcccaactccaaactctttggcatggcccttcagaatctgatctgatccaagaccagccaggagagaggcagcgttctagctgtgtgtcccttactctccctggggttcagtttcctcctaataatagggtggttgttaagatgaaagcagagcagcacatgacacatgttccacaggatactagtcaatacttagtagatgtgggcaattcttattacccttctaatgccattctccactactcctaagtaaaagttcttgctcagattaaaaggtatttggtgaaaatgttttccccactctttgtgaatagacttaatccgttagacagtaaccagagtacctaacagagtggtggagtgtacaaggcattgtgctgttacccactcaagacacaggtgcttttattatccccaagatcaagtaaactgcccagggtctcacagtgagatgtggccaagctaagaaccacgcccagtctgtgctgaccttaattacttggctggttgtgcttccagatgccatcatacccacatcagcagctgctatctagaagcatcacattttcctctgtgagatctacagggcctgagtacaatttgccttatttttcagagtcctaatccacggtagaaagcgtggtgtttatagaatactgcagatggcaccaagtctttgatgttttcttcttaaaattgtatagtccttaggtaacgatagtagccatgatttcttgaatgcttactgtgtttgagacatcatatacatatcatcacaaacacccctatgagataggtactgttgttatccccatcatatggacaaggaagctgaaactcagaaaggttaagtagcattcccagagtgacagaagagccaagtagaggacgctgaattcaaacccaggcagatggacgtaaggcctttgcttttcactatacattacacaccttcccccagtctcaacagagccaagtgtcagacactcatgattctgacttcagcatccattctgttaatctctgcactattctgttttatagttttctttgggtgattccaaaggatttatttttaaagcatacctctctccagctgaatgcctttcatttattcaccagcaaaacagggtataaagtgaaaaggtgttgaccaaaaaggctttcacttttttcaactggagtataatatttatcacggcttgtattacgttggatgataaaaggagagatgttgagttggccagatgagggagatgggaagagctattttctgtaaaggcaaaggaaaaggtgaagcaagggtggcatgtcctgagggcccctaaagcgctgaccttggcattggatcctggttctgagccctgcggtgattccaaatgtgaccatcgtggagaaggcacctcagccaagaccagccctccttcagaggacagtcacctccagagcacctccctaggggcagcagctatgtctgtgtccccacacccagcacctggcattctagagatgcttcacaaatatttattgaatgaatcaaagaatcatagcagtgaaaaagagagtctattgaaaagatcaaaatagtcattgcttcagaaggcagtgggtaccagcattcactttcctcctgtcctttgtttggttcctttaatgttacctatacattattatcattagctgaaaaatcatgaatcttacccattgaatgctcgtactttaatctgtctttcctactgaactcaattcagataaaattgcttgttttggaaaaagtctccaatagtcagaattttttaagccagttgtgacctctgttcctttttcttctctgcagcatgcaagtgcaatgggcacgcgtctctgtgcaacaccaacacgggcaagtgcttctgcaccaccaagggcgtcaagggggacgagtgccagctgtgagtaccatactccctggaccaccagggaggaccaagaggctgtgcagctgcctgaaccccaccctgagagccacccacttccctgtgtcttgttgctgtgggctctgagggatccctgggttgattagtttgaaattttgcccattctatttcagacaggtcagtccccaaaatgaggaggtcgtcgagttagcagcaattccttaatggctcttgaattcacatttttgtttaaatgatactgacatttcctgggttgtccatttggagtagtcattttaacttcagcaactacttgatttttgtcatgtcaagagattatactctttcccaaagagtagagatggaagagctggtgttttggtgatggcatttatttggcgtttggttgtctgattgtggaaatgatcctcttacctttaacatttcccattactctagcattttccttgttgaagcatgtcaggcatcttcctggagagggtttgaagtccccatccatgaggatgcaggtgcagcagcatcaggcatgtttaaggtttcagacctggccctgcctccactgagcaaggtgctcttgaggcaaatcactgttccccccatctgtcagtagtgggactagcagtaattgtttttggtgtcctggtaggttgaaacaggaaggatagttcctcaatcaatgtataccgggctgtctcactgggaaacctcataaaatgcatgtatccgcatttactcattctcaagaaaaaacttaaaaagtgtttagtgtcaaaacactgagctgattaatcattgtaaacatcattatttcttaaataacagtagtaataatgccaccagtgattgagacatcattgctggtgctactcagcagggtttggtctgtctactttttagtttatatctgcatatatgtttactagcttcatgttgccctaaagccaatatgtgaagaaaagtctaatccaaggttaaatttaacttttaggtccacattcccttaaacagaaaaatgtcatcgagcaaaattaatcactcttaccctcagttagctgatgcaacaaagacatgaggtggcatcaagtaagactttcaagttccttcgagatcgcagatgtaggcaggtgctgctcatcaccctggctaaagggacacagcatacctgcctgggaaaggccattacccacttcccgccctttcctcttcatgcatagctgttgtgtttacctaaatgcttcatgaagtgggaggctgggttttgctgatgtttatagatgatctgtatggggaaattgttttctgagtagaacatattttttttctacagactggtgaacacttgttccagggtttaaaaaaacagtgattcttggtatttagtcttctctcacttgtgtttaaagaagagaattagtttatccagggaacataggaaagaagtgagaaattagtatttgaaaaatgttttgactgcacttttagaagaatatgtagtccacacaaaattgaaaatgtttttcattatagtaaataggtttattacatttggatttctctcattgctttggcaaatgcttgtcaattgtcctcatatagttggtgtgtgaactgctgagcagtcagtattgaagcgttcatgcatgctcttcctagactgcctgagctgagttatggtgaaggatgcaattataatggctccagactatctgtactttatataaaaggatctgtttggttttaaaatgaattcagtttctgttttaaatagcagtataaaatagtcttttttcccccccagatgtgaggtagaaaatcgataccaaggaaaccctctcagaggaacatgttattgtaagtggttttgcaattcttatttctagaagcaaagtagttcagtaaaacttcattgtttaaacgggtttgagaatagtaagtgctataagactatagcagccaccaatgaagtgttcccagacttgatatgtttacattctgttaagtttactacatataggagcactgttttaagctgttttaattgtgtttggggttaacgttaatgtgtccatagcagatagcaggagagagtagagaggcatgcatctttgtctatccacatttatgttctcttaaaactttactttatttgccattacctagttggggtcatcatatttcgtgttttaggatgtagatcaaaaacagaattcttacaatatggttgaactttttgtcattctgtctggaactgatttgtgttaaccaccttgaggtgaaaggtgagtctgacaaggtgaccatgtttttatgggtaaatgtgttttctctttatgggagaatccacatggtagaccagagtacgggaccagaaaaaaggagttaatgttatggcatatccattgtaattatatacctgctgtactgggttgaataacatcccttcaaaattcatgtccacttggaacctgtgaatatgacttcatatatatatatataaaacttctaaaagaaaacaagagaaaaacatcttgaccttgggtggggaaaagttgttagataggattctaaaagcataattcataaataaaaagtagactaatgaaactatataaaaatttaaaacttgtaggaaaaaaccatccaaaacataaaaagccacagactagagaaaatactcgcacatcatcatatatttgataaggagtttatatccagaatacatgaggaactcttacaactgaataagatgacaattccagttaaaaacaggcaaagatttgaaaagacatttcatgaaagaagatattacagtggccaataagcacattaaaaacgacttaacatcattcgttatttggaatggagattttaaaaccacagtcagatacagccctaaagtgattagaatccaacgctaatgccatggctttttagaagacagtggtaatctcatgtctgcttctgcattcagtctgttgcagtacatctttttggttaaacacatgaaaaaaacctggccttaccaggcatgcagttggaaaagggtatagtgatacccttttaatagccttttcagataattatggacgttacttgatattatgctgaaactggacaactggtcatttctttaaagcggtttgatgggttaacccaaataaaatgatactgcccacagacttgattttctttgaggaacctgggtacagctgagttaaagtgctttcctcgttacttaatttataagaaaagcagcctgtatctctaaagacctgttctattttgtgtgtgtttagtttttaaatatgcatttctttctttccatagatactcttcttattgactatcagttcacctttagtctatcccaggaagatgatcgctattacacagctatcaattttgtggctactcctgacgaagtaagattttttaaagtcttcctattttgttttgaatttgtatggatctttttcttggtcattacggatggacgtactgccttaacagtgctctccagactggagtacacgagatgatctctagaggtataggaaagaaatgttagactctacgttatctcctttccacataaaaggcaaaagtgatgttaataagatttaccaggatcttagacacagactgacattgattccacgcatacttactctgcctgtcagcccatcatggcctcatacagaaaggggactccaccatcagagggcagatagcagagcctgggtttactctctcaagagtgaccagaggcttaaagacactgctgattgaaatgccagtgatgcagccccaatcagacagcaagggaggggaccccaatcagacagcaagggaggggaccccaatcagacagcaagggaggggacactctgctcctagagtgagttcttagcctcattaggaggcaaaacagcaaaggcttagttgggtccattaagaagttagccaggaataatttaaattgttaaaatatgtatgtaaaatgtggatttttttatctgctgtcattaacgatgaagcccaacctgccttaaggtattacctagtggtagaaggaaggccacactgcgggacatttaaaactgaaacatacagaacacgaagatgcacctgtacagtttcttcaatgaaatataaagtcatgcagtacccacttcagtatttaaagaaattttggtaaacataatggtaaattatttaggaacttccttggagatttcttacttctcatgaacatacacaaagccatttaccattacaaaattccattaacaataaatgtgacaaataatacatggaaaacaatatggcagtaagacaaagtatattgctttgttaacaaatgttatctataaacattgctaaatttaattttaaaagtagaacaaagtctatagtgtggtatgtttactatacaagtaaatgaaaataggatttgtttttaatattctatttcaaagataaaattaagaaaaaatgtactagaaaagatacattctcaaaagtaaattgctttttaagtaaaaataataaattactttaaatgaattattttatggaaaaactataggtattaatatatcatttgagtggctgtattgactggaatttttttcttaacgattttagaataagattttaacaaaagtaccatatatgaaatgtattcactgcctcataagcaagcgtttgaagtgctcaaaatctttcaggatatacttcatgccattaatgtcattaaaaaaataaatatagtagaatctttgtaatacttcttaaccaggtgggaagcaaccatgaaagtatttggacctttctggatttcccagtttatcttacgacagaatacttactagagttatccaaatgcatatgttctgtcctctataaaagcacaagcattttaagtttattgattctttctgtggaaagacatatagttgacccttgagcaatacaggttaaaactgtgaggatccacttacacgtagacttttttcaaccaagcgtggaatgaaaatacagtatttgcaagatgtgaaacctgagtatacagagggtggacttttcatatgcaagggttctatgggcagactgcggactggagtatgtgtggatttgggcatgctcagggggtcctggaaccgatgacccacgtataccaaaggatgactgtaattgttagttgtgtgctgccagcaaatactaaatacaaataagtaaacacttgagctgtcctttcaagatgaaggtgaggtcttatcagtaaatgagaaggtaaatgctttgtgagagaaacttctggtttaatgatcacattttaaaaatagctgtttggaaatgtttccattgttgtgattttgtgctattaaaatgatcaaaacaacacccttaaaaatcttattctaacctctcaagatcttttaaaaatgaataatttcagtacagtcggatgcatctgtaaaagataaaaatataacattgattagtttgcaaaaataattgtttgaccccagttaagagatgtactagtcaaatttcagtttgactaattattaatgttataatttacctaacatcaccaacagtacacttcctccactggcttaacagattcctcagcaatatctttattagtcattaagaccaaggatcaaaataaattaagttagattagccctgtgaactgctatatctctaagtggtagacaaggttttcaaaactaagaagccatactcaatcatatttctcttgaaataatatttttagtaagagaaaaatatttttcaaatcatgaatataataaaattattttttaaattaagtacatttcagctctatatgccttttttaaaggctgtttccagttttggagaagttttacactgtatataatctatgagtttagaattatatgggtttcagttataaataaatataattttgggcacttgatcaagtgttttattggtaggagtgaacttcagacatttgaaaacagctgcccagagaatccttaggagggtgattccccagcacaggtcaggagatgcaaatcactgtgctctgataggggtctttttaaaaggcactttctcatgcagttaggcatttgataaagcattaaattatgtatactttaatgggagggaataaaattttattggcatatattgcttgtattaaggaaaattggttagaaattttgctaactcttctgtgagtttctctaaagacatcataaaatgtttgtgattaagaacatttagaggagtaaactttattgctttattttaaaatctagaaattgttttaattaattttctaataatttgtaccgctcatcagcaaaacagggatttggacatgttcatcaatgcctccaagaatttcaacctcaacatcacctgggctgccagtttctcaggtaaagacatacctagagaagaccctgcaaatgaaggtgtggtagattaagaaatgtaatataggaattgagaaagcgagctcaggagacagattggtttgaaacccacccttgccacttactagctatgagaccttgagcaagtatctaaatccctctctaaaccttagcattattttattcatctgtaaagtgaggataatgatacttacctcttagaattgttgtatagattaaattaggttatacctaccagagcttgctgtggtgtcaggctcagtgtgtggttactaccctagcccaccaccaccatttctgttcttgctgtggccactggcactaccatcattgtctacatccgtgcttcggaagtgaaaaattcaaatgattcgtttcaataaatgaaaacattttaaataaaatgagattttagtaggtacagagaaatgtaacttgggaattacatcaagctctaaaagcacagctcttgctgtctgccttactgtgattcactgaagatctactgtatagaaaatctaaagaaataaaggatgaaggccaagtgctgtggctcatgcctttaatctcaacactttgggagactgaggcaggaggattgcttgagctcaggagtttaagaccagcctgggcaacatagtgagaccctgtctacaaaaaaaaaaaaaaaaaaacccaggtgtagtgactcatacctgtaatgccaactactcaggaggctgagatgagaggatcacttgagcccaggggttggagattgcactgagccatgatcgcaccattgtactccagtgtgggcaaacagagcaagatcccatctctttaaaaaaaaaaaaaaaaaaagaaaaaaggataatcactacttaacttgataactcaacaagtagatatgggtttgaaatttgtccattaaatttacttgcaccgtgctgttaggcaagttacttaaggtttctgagccagtttcctcctgtataaagtaggatagtaaaaacaccgtcctggcagggcgtgatagctcatgcctgtaatcccagcactgtgggaagccaaggtgggaagatcacctgaggtcaagagttttgagaccagcctggccaacatggtgaaaccctgtctctactaaaaatacaaaaatcagccaggcgtggaggcacgtgcctgtaatcccagctactcaggaggctgaggtaggagaatcgcttgaaccttgaaggtagaggttgctgtgagccgagatcacgccactgcactccagcttgggtgacagagtgaaactccatctcaaaaaaaaaaaataaaaaaacaccttcccaagtagagtgatgtgagaattaaatgagataataaatgaagtactcaatatagtgcttgaaatgtggtaaatggtaactatattttatcattattactattacaatactgggtttttaaaaatcaaaaacacaaagcaatgagattgatgcaaaataagaatattgccttgtgcacgccacttacgtttatcatcttaaaacattgtgtagaatttgagaaaagttcagaaactctcaatgaggagggacttttaagaaaaagtctgaattatcagagtatttggagaaaggcaacatctccaggcatgtgaaagatttgcaatgagccgggcggtggctcatgcctgtaatcctagcactttgggaggctggggcaggtggattacctgaggtcaggagttcaagaccagcctgaccaacatggtgaaatcccgtccctactaaaaatacaaaactagctgggtgtggtggggcgtgcctgtaatcccagctacttgggaggctgaggcaggagaattgcttgaacctgggaggcggaggttgcagtgacccaagattgcatcattgcactccagcctgggcaacaaaagcgaaattccatctcaaaaaaaaaaaaaaaaaaaaaaagatttgccatgagtgtctcaatgaagacgtgataatgtgggctctagtcacagggtctaactcagacatggaaaaaagtccatttcattaatctttatcggcacttgaattcctggctaagggagaatgtggaacattgaaggactctctgggaataggatggagttataccagattagggggacttaaatactgtggtagctggtggtagaagggaggactgagtgaccccttgaacccctcctccctgctacagtgggttaggcagtgagcggtacatcagcattactggcatgggagtctggcgcattgccaaggaggtgtaaaggggaaatgcaaaggaattgaagtggtgtgggcaaagtgaatgccagtgcttgttaataggattctagtggtatctgtattttcatgatcatgtgtgtcacctgtttgggggtggggcaagggtggaagggagttacatggattcctggtaaaaccatttttctttctttctttttttttttttgagacggaatttcgctcttattgcctaggctggagtgcaatggcgcaatcttggctcactgcaacctctgcctcccaggttcaagcgattctcctgcctcagcctgctgagtagctgggattacaggcatgcgccaccacgcctggctaattttgtatttttagtagagacggggtttctccatgtttgtcaggctggtctcaaactccgacctcaggtgatccacccgcctcggtctcccaaagtgctgggattacagatgtgagccaccgcacccggccagtaaaaccattttggttaggggcataggcttgtatatcagcctgcccagctttaaatcttatctccatttcttgttggctgtgtgacttgagggaagttacttaatttctgtgaacctcaatttcccagtctataaatgaagataataatagggcagctgtgaggattaaatgagattgagctcttaaaggctattactggaacacaggaaatgtttgataaatgctattgtccttattattaatgaggccagattctgttctccccctagccccccaaaaaatgtctcttctctttcatgtttttcttttaacagctggaacccaggctggagaagagatgcctgttgtttcaaaaaccaacattaaggagtacaaagatagtttctctaatgagaagtttgattttcgcaaccacccaaatatcactttctttgtttatgtcagtaatttcacctggcccatcaaaattcaggtaagaagaggcttttggtctcatacctgcaaaggtggtgaaatctctttagtaagactaaatttactaatttggagcttgtggtaaatgagatgtgcaatgtggctttgcctttgtaacgtgtatggcagaggagtgctgagcacatgcatgctgcacagaagattggagtggggatggactgtatcactcatgaaagacatttgcaaaagcactgttgaaagcaagttggcatgtaacagatttggtcattataaacgtattcacttcttcagtgagcatttgccatgtgaaagcctgtagggctacacaaagaacttcaattctagagtaaggtggatgtaagtgaaacaaacccacatataacaactgaaagccagagtgtggaaagtgacatgagatgatcccagaaatgctatcaaagtttaaaggaggacaaatggggagactatgttgaagaacatcagccttctagtcagacagaggtggattgattcctggctcctatacaaatcatacagcctttccaagtctccagttctctgtgcatgtgacctgaagggtagttgtaaggggctgtgcagctgctgcagtgtcttgttagcctgctcctttcctctgttcccagggggccagtgtactccctcttgtccgagacccatggccccattttaactttttatactcatgtcccctggggcctttcctcaataccttctgcttcttaccttcttcatttaggtgaatgtggaggttagggataggtgggctttcaaggactggttcacctttaaccatggaagcatggtcactggacggaggctgttgctgtttgccaatgttcagaagcataatcaacctcagaagcaagtcaccacaaacatatgaaaaaagttcaacatcactgatcattagagaaatgcaaatcagaaccacagtgagataccatctcacaccagtcagaatgtctgttattaaaaagtcaaaaaagaacagatgctggcaaggctgtggagaaacaggaacgcttttacactgttggtgggagtgtaaattagttcaaccattgtggaagacagtctggcaattcctcaaagacctagaggcagaaataccatttgacccagccatcccattactgggtgtatatccaaaagaatataaatcattctgtaacaaagatacatgcacatgtatgttcattgcagcactattcacagtagcaaagacatagaatcaacctaaatgcccatcagtgatagactggataaagaaaatgtggtacatatacaccatggaatactatgcagccataaaaaggaatgagatcatgtcctttgcagggacatggatggagctggaggccattatcctcagcaaactaatgcaggaacggaaaaccaggtaccacatgttctcactcgtaagtgggagccgagaacacatggacacatggtggggaacaacacacactggggcctattggagagtgatggggaggaaggagagcatcaggaagaatagctagtggatgctgggcttaatacctaggtgatgagatgatgtgtgtagcaaaccactgtggcacacgtttacctatgtaacaaacctatacatcctgcacatgtacccctgaacttaaaaattaacaataacaaaaaaagcaagttatcactcatcaattaggatgccttgggactgtgactaaagaacagttgatctttccattctggaaatatggaggacaaaaactatgttgtagtttttctcaccaccctctctcctttttcctgtctccatggtctaagatttgttaatcctctcatcaggttctccctttgccctcgcatactccctgtgtccctcctcagccagtcttgtaagcatcagccacgcttcttattcctgttttctcttgtctagtcacacatctgctcacaatggtctggcctcttccctcaccacatcctgaaactgtcctgtcatagtgggccccagtgatggaatttttcagtcccctttttatctcattgcatggctttgaatcatcatctttcttgtcttcctggatttttcttttcctcgttttctggccagtctttttagggaatcctcttcctcttccttaaacactgggcttgtctaggattcagacctggtccttttctctcttcactctgtttcctgcatgtgggacttgtggtgccacatctattctggtgattctcagtgctgtctccagaccggcactggcagttgctgcctggacagccctacctgggtagtcaaagctccccgcgttcggtgcatattccttcttgtgatcttagccccagacttccatgcttcctttctctgtaaacagcaccaccatcaccctgttgtacaagctagggcctgatgatcatttgcattcctgggataaacccaacttgagcatgacgtattatttccttactttacacgttgttggattcagtttgctaatatttaggttagaattttgtatctatttcatgagtaagaatgtctgataattttcccttcctgtccttgttatggttttgatatcatgttaaactaacttttaataattataatttgaggagttttcactctttctcatctctggagatgtaagattagagttaactgaacctcaagtacttggtagctctctcctgtaaaatcatttgatcctagtgtcttatttgtggagatactttttagctgctgattcagcttctttaatagttataggatattttgaatttcctctttatttgattcactttttaatattttttctaggtttttatatcatgtttttaaatgtatttttaattttactttttaaattgatacataattgtacgtatttatgaggtacatgagatattttgatacatgcatacaatgtataatgatcaaataagagtaattaggatatccatcacctcaaacatttatcatttctttgtgttgagtacatttcacatcttctagctattttgaaataatgaataagttattgctaactatagtcataatgactattgtgccattgaacactagaacttattccttgtaactctatttttatacccattaacctctatctccccagccccccagcagctggtaaccaccattctgcactctacctccatgagatcagtttttttagctcccacatctgagtgagaaaacatatctatctttctgtgcctggcttgtttcacttaacataatgacctccagttccattcatgttgctgcaaaggacaggattccattctttttgtgactgaataatatttcattgtataatatatacaacattttatttgtccattcatttgttgatggacacgtaggttgattccatgtcttcactcttgtgaatagtgctgtgataaacatttagagcatgcagtatctctccagtatactgattttctttcttttggatatatacccagcagcgggattgctggatcatgtggtggatctattatgagcagtcttcatactgtcttccatagtggctgtactaaataatttacattcccaccagcagtgaactagtatcgtctttctctgtatcctcgccagcatctgttattttgtctttttaataatagccattttaactgggatgagatgatttctcattatggttttgatttgcatttgcctgatggttactgatgttgagatttttttcatatgcctgttggccatttgtatgtcttctttggacaaatctctattcagatcatttgcccatttttaaatcaagtttttttcctattgagttgtttgaattgctggtatattctggttataaatcccttgttggatggatagtttgaacatattttctctcattctataagctgtctcttcactctcttgtttcctttgctttgtagagctttttggcttgatataatcccatttgtcgatttttgcttttgttgcctgtgatttcgacatcttacacaaaacatctttgcccagaccaatgtcctgaaggattttcccaatattttcttctagtagttttatggttttaggtcttatacttaagcctttaatccatttaaatttgatttttgtatgtggtgagagagaggggcctagtgttatttttctgcacatggatatccagttttcccagcaccatttattgaagaacctgtcctttcccccactgaatattcttgactccattattgaaaaccagttggccgtgaatatgtggatttatttctgagttctttattttgttccactggtctgtgtatctgtttttatgctggtatcatgctgttgtgggtactatagctttgtagcatattttgaagtaaggtgatatgatgcctccagttttgttctttttgctcagaaatgctttggttggctgagtacagcagctcgtgcctataatcccagcactttgggaggccgaggctggtggatcacctgaggtcaggagttcgagaccagcctggccaacatggcaaaaccctgtctctaataaaaatacaaaaattagccatgtgcagtggtgggtgcctgtaatcccagctacttgggaggctggggcaggagaatctcttgaacccgggaggaggaggctgcagtgagccaagattacgccactacattctaccctgggcaaacaaagcgagactctgtctcaaaaaaaaaaagaaaaaaaaaaaaaagaaatgctttggctatttgaggtcttctgtggttccatacaaattttaggattgttttttctatttctgtgaagaatgtcattggtgtagagattacattggatctgtaggtagcttttggtagtatggtttttttcacaatattaattatttcagtccacaaatgtggtgtctctttcaatttttttgtgacctcttcaatttcttacatcagtgttttatagttttccttgtaaagagggctttcacctccttggttagatttattcctacggttgtttttggaagtttttaaaaaatctatcgagaatgggattgctttcttgatttcttcttttgctagttcgttgctcctatatagaaatgattctgatttttgtgtgttgattttgtatcctgcaactttactgagtttgtcagttcttagtttggtggagcttttagggttttctgtatataagattatatcaactgcaaatagggacactttgacttcctcctttcagtttggatgccctttatttctttcttttgcctagttgctctggtcaaatatgttgataacttgttgatgctgtcctctaatgctcgcagcgtctgtgttcatgaaaccctttgttgtattccaatgttgatagcattattcacagtaatcaaaatgtggaaacaacccaaatatctatcagtggatgaatggataaacaaaatgtggtatgtatgtgtgtgtgtgtgtgtgtgtgtgtgtgtgtgtgtgtgtgtgtataataaaatgttattcagcattaaaaaggaatgaaattctgatacatgcgacaacacaagtgaaccttgaaaacatgctaagtgaaataagctagttgcaagaggacaaatattgaatgattccacttaacatgaaatatctagagtagtacacagattcttagagacagaaagtggattggaggttaccagcagctagggggagcgggaaatagggaattactgcttcatggttattaaagagtttccctttggggtgatgaaaaagctttggaagtaggtagtgatattggttgcacaaaattgtgaatgtaattattgccactgatttgtacacttaaaaatggttaaagtgattttatgttatatcttactacaataaaaaaagtctttaaaaatctcagcaagatgtttttgtagatatagacaagcttattctgaaatttatatgaaagggcaaaagttctagaatagctataacaatttagaaaaagaagaataaaatgggaagaatcaacctacccaatgttaaggcttactatttaggtacagtaatcaagacattgtttcaggtagaggggtagacacacagatcaatgggataggctgatctataggtagatcccagaaatagaaccacataaatatgtccaactgattaggatttgattttgtttgtggtgtttttttgagacagggtctcactatgatgtccaggctggtcttgactcctgggctcaagtaaccctctcacctcagcctcctgaataactgggattacaggtgcacaccgccacgctagttccagctgaattttttttttttttttttttttttgagacagggtctcactttgtcgcccaggccaaagtgtagtggtgcagtcacagcacactgcagcctcaacctccctggctcaagtgatcctcctgcctcagcctcccaagttgctgggactacaggtgcataccaccatgcctggctaattcttttttttttttttttttttttttttttggtagagatgagatctccctgtgttgcctagtctggtctcaaactctcaaactcctgggctcaagtgatcctcccgccttggtctcccaaagtgctaagattacagacatgagccgttgcgcccagcccccagctgatttttgacaaaggccctgcaaaggcaattcaatgaagaaaggatagccttttaacgaatagtgctagagcagttggacaaccataggcaactaaaggaatctctaaatctcacatcttatataagaattaacgcaatattgatcacagatctaaatgtaaaacataaaactataaaacttttagaaaaaaatataggagaaaatctttgaatcccaaaacgaaacaaaagctttttagactcgatactaaaaacgtgattcataaaaggaaaacttgataaactggaccacataaaaccaaaagtttttgctctgtgagaaaccctgttatgaagatgaaaaggcaagctatggactgggagaaaatatttgcaaaccatatttctgggaaagcacttgaatctagaatataataactctcaaaactcaacagtaaacaaacgaacaattcagttagaaaatgggcaaaagacatgaatagacatttaaccaaagaggatatacatatggcaaataatgacataaaagatgatcggcatcaataaccattagagaggccaggtgtggttcacacctgaaatcccagcactttgggaggccaaggcaggcagatcaccacttgaggtcaggagttcaagaccagcctagccaacatggcaaaaccccgtctctactaaaaatacaaaaattagccaggcatggtggcacatgcctatagtcccagctactcaggatactgaggcaggagaatcgcttgaacctgggaggcagaggttacagtgagctgagattgtgctactgcactccagcctgggcaacggaatgagactgcctctcaaaataaaaataaaataaccattagagaaatgcaaattgaaagcacaatgagataacactacacacttatcaaaatgactaaaattaaaaatagtaataacaccaaatgttggagagagtggggaaagattggatcactcatacattgttggtgggaatataagatggtacagtcactctggaaaatactttgtttcttaaaaaactaatgttatacttatcatataacccagcaattgcattcctaggcatttatcctagagaaatgaaaaccagtgttctcacaaatctctgtacacccgtgtttatagcacttgaaagcaatatttaacagctttatttataatagcccaaaactggaaacaagccaaatattcctcaacaaatgaatgcttaaataaatgatgctacatacacaccatggcctaatacccagcaacaaaaagaaatgaactgttgatatacagaaacacaacaactttgataaatcccaagcaaattttgctgattgaaaaagaaaatcttagaaatttatatactgcatgattgcatttatataacatttgaaaatgaaaattttagatcttatatacctatgcacatattgaataagtatcctgaagcttcaaatttctctgacttcttaggactctgatctctagaccctgttttaaaggagcttatctgattaggtcaggcctacccacacaggagtagggaagatacagagcggtgcccatcaagaaggtggaaatcttgggggcattcttagaattctccctgctgtaggctgcatttggaatagagcttgcaggggtttcggtagtttctccgatccgaaaaaggttgagctctaggtcccagtcagtataatttcagtttggcaacatctcctgacactattaagatatagattatatatatttgttaggttattttcatgtaaacacttgattagggttctagtaaataaaatttatttatttactcattttaagttgagattgtttttgtactgacatacctctccatattctcaatctaattgaacatcgtgctttgttgcttatagctgacaaatgattaatactttttagattcttagtgaaaatcttgacttatgctgctgacacttttcccagtctgtgcctataagcaggcttttgtgctggagatggccatacttgttgccaatgccgagtttttttcctcccagttccctattgctccaccctctgaaagtcactgaagtggttttagagctttcattctagagggagctcttccccacacttacttctaagaagtaataattttaagtttcataatcaaggagatgttgattttcttcctggttttctggtgctccttatcttttctggatcactaacttaaattaagatccacttatgaatgctgcattcaggagcacttgtgaaaataaacgatggcatgcccaccatttagtttgccaagctttccttgaaaacgcaggtaagttctattgcaaacgtgtctttttgctgtttggtacaaatgactgcactattcactgtgactaggcccgtgtcaccgtgtgttctcagtcctcgcatgagagcctggttgacactggttctctctgctttggggaccgtatctggctgtgcgctatgtggagggctgagctcgtgtcttcctgggtaaatacatcatgttctcaacaagggctgcatgtttctttattgctgttttttccccagagacttagcttttctaggtgtgtagcatggctcttaaatacacacatttagcaactgtgcaaattcagtaaaaatggagagacttatttaaggaagacagtagggaaatgtctaaatagtgcagtgtaggccattcagttaaatttcagtatggctttcgatgcaggcaaagccgttttgcctttgattctcattttttaaattagattttaaaaattgagattctccttttttaaatgagattttaaaaattgagattctccttttttaaatgagattttaaaaattgagattctccttttttaaatgagattttaaaaattgagattctccttttttaaatgagattttaaaaattgagattctccttttttaaatgagattttaaaaattgagattctcatttttttaaagcaaagtaactgaggaaatctaattctctacttggaaatttgtgtaagcattggggctttaaaaatggtaatactaaactcaagttccttcactctgaaccaatcccaggattcttttccataattaggaacagatcaaaacctataactgctgagggaatttgaaactgactataaaaatctttgtagatgcagctaaaaaacaaacaaaacccacagactgaaaagtcctgtttctgggagggtggcgggtgtgttggggccggttgtgcctgttttttgattggttgttcctgtgggtgagcacccttccctctgcccctgctttctgaaagctatcctagttgttctccccagtaagccggggctcagccggcttcagctatgtccatagtttgggggcgtgggcggtaggtattcaagatagaagatggttggacagaaagtgtgtgaggtgggaaggtggagtggtgaggctctggctcgttcccgactctgcaacagaggttgttactgtaatgtggagctaatatagaagtctgcgtgctgggtgtttcatctactgttttatgtaagaggccagtgttcacaagtaaaggcaacacagaattggtattgagaactgggcattcatttagattttagagttctgtttatgggtgctaacaggatcgctatactgagcaagggatgtgtgaaacttttgttcacttggatgtgctttttttcttataacagctttattgagacatcatttacataccatacagttcgctcatttaaagtacacaattcagtggttttcagtgtattcacagaagcgtgcagccacccgtcactacagccaattttagaatattttcaccacttccagaagaagcccctagcagtcagtcctcattcccccctcttccggcaacccccagtctgtatttcctgtctctgtggatttgcctattctggacattttatataaattgagtcacatgatatgctctcacttagcatactgttttcaaggttcacccgtgtttagtatgtttcagtacttattccttgtcatagctgaataatcttccttatatgtatataccacgttttgtttatccatttttctatcgatggatgcttgggtggttttcactctttggctattatgaatagtgtcagatatacttttaatgtttatatttattgtataataacagtgcatatccgtacctcctggtgttaattgtctgtatcttaagagttttaagaattatatggttgtgctgtaattctagcacttagggaggccgaggcgggcagatcgctttagctcaggagtttgagaccaggctgggcaacatagtaaaaccctgtctctactaaagtacaaaaaattagctgggcgtggtgaagtgttcctgtagtcccagctacttgcaaggctgaggcacaagaatcacttgaacccaggaggcagatgttgcagcgagccaagattgcaccactgtactgcagcctgagcgacagagcaagactcctgtctccaaaaaaaaaaaaaatttttctttatgatgaaacccacaaggctgtacaagaaccttaaatatagtttgaaacaaagaaagacacatgcttttaagtacagtttaattcttttcttttctcttatttcattttttttttttttggagacagagtctcactctgtcgcccaggctggagtacagtggtgcaatcttggctcgctgcaacatctgcctcctgggttcaagtgattctcgtgcctcagccttgcgagtagctgggactacaggaacacttcaccacgcccagctaattttttgtattttagtagagacagggttttaccatgttgcccccatcattctcagcaaactatcgcaaggacaaaaaactgaacaccgcttgttctcattcataggtgcaaactgaacaatgaggacgcatggacacaggaaggggaacatcacacaccagggcctgttgtggggtggggggaaggggaagggatagcattaggggatatacctaatgttaaatgacgagttaatgggtgcagcacaccaacatggcatatgtatacatatgtaacaaacctgcatgttgtgcacatgtaccctaaaacttaaagtataattaaaaaaaaaaaaagaaaaatgtgttattcctttgtctcgaaagtcccctgcttttcagcttttgctttggttgtctcttgtcagctctttaatgctattgttcccttgggtttcatctggactctttttccctgggcaatctcatccacaccatctgggaacattgcctttggctggacaggtaaatctttgtctctagcagacttctctcctgtgatctccagagccaggaacccactagacttccttaaaattcattcagccagcagactttctggcccagagcagcagctgtgagtggacagcaggcacctcgttagaagcttggtgttggaggtgtgcccatgcattcctgggagccatgagaggggaggggagtgccctgtgtcacctgagtccaagttaagagagatcacaccagagctgattcctaaggagcatgggggcgggggaagggggtgttcgtggtggggaaaggaaatgaccagccctttcaggaactaccaggagtttagcagagctggtgacagtgctatgagcaagaaggtggagagctggggagggccattatgaagagactggagattgcaggctgtgaacttgagtcctgatgtgactgggaattgttgagatgttttaggccgaaaagtcaccgacgatcagtgtttagaaagaccccactggcagcagttgggagtgtggattggaaggagccaggcctaagtcctgggaggacttaggaggtccaggagtccagggagtccaggtggaattcttgtgtagaaacccagggaaggggaccatctgatgacacacatcccatagcaacttcctttcctcctctgcacctccacagcagagtgtctgtttccactatacgaagatttcttaatggcaaggatcgcatttttatccctcagtgtcctcagtgtcatgtgctaatcagttggagacactccctaaatgtatgccagatgagtatatagttgttagtataagggctttacggtaggtatcagaactttttcttttttacaacttttataacttttataaaggagccgacacctttttctgatattgtcagaacaattgctgaccagtgctatgggctggctgaggttggcaaccaagaatctgttatctcagtctacacagcgtggagttagaagattggattgttgttggttagaattttgtccagagagttagaagatagaagaaaagggagggctgaggacataaacatccaaattttacaaagaagaaagttcagtgctcctgtctatattgaaatagttaagagggaaatgattgggtctggggaatgcttcaaaataatatgatcatagggaaagtaagtgtagtgtggatgaaacaaggttggccttgagctgataatttactaaagcaggatgatgagcgtgtgaggttcattatagtgctctttctactttttaaaaagcttaagattttccaaaatgaaaaagaagctgagcatggtagctcacgtctgtaatcccagcactttgagagcctgagacggaaggatcacttgaagccaggagttcgagaccagcctgggcaatatagtgagacccccatccctactaaaaaagaaaaaaaattacacaatgaaaagaataatacaggtggtagaagaagaaataaaggtatgagtatatgatttggcctcctacagacagctacaggagcatgcctctgctgggcgggcctggagcatagtgcaccctggagctgaagttctgttcgcctttacaggaagtacacagcattgtacgggggttttaccagttaacagtggacagtctctagaacactcaggggtcaggggcaaaactccctctccctcaaaccagcccacctgtcttggtgagaatggaaaacggagcttcccagcaagcctcctgccttcaccccgacgcacaggtgcatcagggtctgagggctcctccctgtggggcctgggaagccgcccttcagtgggctatggctgcattgtcttctgatcttgtagctgtgtgtgggtaacaggcacatcgtctccccatatcccagagagtcatgtccacttcctatctgtggtggcatctttccatcactgtcaccctcaggataatggcacaggaactaacagaggcactaagagcagtgatatatactggagggaggggcggggctgatgaccaaaatcaccttctgtttcagtaagaggttggttaaagtgatgcctaaaatggatagatcaggaaataacaataaaaacattaatgataaaaaggcaactgccagaagaactgaaaagtaacctagttcaaggtgggggtccctggagagaagccttcagaagagcagaggaagagcagggaacattgcttttcatgactagcctttcagtgccttttgatatttcagctaagtatgtagttctctaatgatttctaaattgttaatgagtgtgatagaataggaattctggagaatggtcaaaaggaattgctaggagaaaattagggaaagaaagcacagagcagtattgaggtgggaggttgggagcagacagatggtgagtgttcgtttgcatggcaggaatgagtggctgaatggcattaactggccatggggctctgaccagagcttgggtatacagtttctgtgccagcacaaaccagcattaggtgcgtggtgtgtgaggccgggtgatggtcatatggatctggtccagagctgtggtcttgacatagagttccatgtatgaactttggtggtggtggtggttttgtttcgttttttaagaaaaaaagaagagttgttttatgtcacagccaaactcaaggaggtgacagcacaagacccaggttagaatgtacttagccagggctcacggaatcttccatcctttgcaaagccaagaagagtagaggcctcaaggagaatagaggagcacatgggctccttctgggctgagctctcacccttggtggggccgctggtcccagggggtcgttgggagcctgcggagattctgcccagtacatacataccttgcgttttctcggagcctgacaggtgctgccttgaagcggaaagacttcacctcacagggtggccagctgtcctttctcttcctctccctgtcctgtgtgctcatgataatggatagatagacatctccatggtagaaataatgcagccctttctgcagtccatggttccacagcatatggtcagtagacttagatgagtgctacataaagtgtaattagttctgcaccacggtgtcttaccttctgaaaaggaaactctggtattgacttagaaatgccagttttatccttccgaccagaacgcaagtcctgaatgaggccagaggccaatgaaaggtcctttcttctctcttttcaaatctgctgtattaaacagtgacttttcctagtcattcagtattcattcattcatccatttattcaagtttttgagtaccttctttgtggatggcacatagtagatgcaaaaatacaatcatttttaaaatggaaagcatttgttatgtggtagtggaatataagaaatgccatgcttccatatttttatggtaatcttggactcaggttctacccaccatacttaattagaaattcattctcagcctctgttttggcagagacccattctcttccctcaggaacatacagaaacctcagggctgaactgagctcagaggttggggaaggtcccctggtctgatcttcagtcgctgactgctgcgcccgattccctggcccctcactcttgagggcagcatcctcactcttgtgtggcacaggcttggacatagaatgacacttaagcaccagagcctccctagcctgccacaaggacaccctacattcccaggcactttcagaatgcaaaacctcccctccattccctccctttccctgcctccaggtttgggccctgcctctccttttgtttcaaagcactgtcctctcttcctctacccttatccttccccatctccctcccactccccaaaagagccaaactaattatggggatttaaccttccaattaaaggcgaggatttttttttttcaatgcagttcctgaaatgctaaagaaattgtccagggaaaggagcacaggaggagttttgttttgttttgttttgttttgttttgttttgttttgttttgttttgagacagtctcgctctgtcacccaggctggagtgcagtggtgcgatcttggcttaccgcaacctctccacctcctgggtttgagcaattcttgtgcctcagccccccaagtagctgggatcacaggtgcacaccaccacgccagctaatttttgtatttttagtagaaatggggttttgccatgttggccaggctggtttcgacctcctggcctcaagtgatccacccacctcagcctcccaaagtactgggattaggcgtgagccactgcatccagcctgatgtttaaataatttcttacttcagagtttcctgaagtgtggcctatggagactagcatcagaatcactggaggagcttaaagatatagagacagggatagggcctgagattctgactttgttgtaagctatccagagtttgagagccatggcctagttcttggaatacaaaagtgtatctgctcttgtatgggaggtaggaagttaccttttcattcattcttctacaacagtaatttaaaagttaggaattggtatcttcatagacagaagaatgcaggaatcctcttctcaggatgtctgtctttacacagccatggtatatggtagaccaagtttgtccaacccatggcccgcgggctacatgcagcccaggacggctttgaatgcagcccaacacaattcataaactttcttaaaacatgatttttttattttatttttttttaagctcatcagcactatcgttagtgttagtgttagtgtattttatgtgtggccaaagacaattcttccagtgtggcctagggaagccaaaagattggacacccctatatagaccataattatcacacgccctgagagggaagcagatcaatggtgagggagcgtagaggagagcctgccagagtttccagtggggccggggatgcttaaaaacggaggcagtgatgtgctgagccagaaaggacagggaggacccccatgcacacaggcagggaggctctctggtgacatctgccgtcaggtacagtagtacagtagtagagtgagcggaagcacttttggctgaagtgcaacattagtgattgaggaagtgcaggtccgtaatatcttaccccaaaccctggggctcactgagcttccgagttcagagtttttcagaattgatgaacacatccagaaggcccatggggcagatacaccttagacatattgatgaaagtggagttggaattgggttgtggaagtcgtaggacgtgaattctaggtttcttggagtgtggagtatggtgtctactgtttggaaacagagaagaatgacattatgagttttatttataaaataagaatttcttctttgtattaaaatagatttttatataggaaattttcattaccttcagtatcagaaaggcccccaaatctatttcaccgtttccataataaattgtgaaagaaagatttgattgggatattgtctttaaaaatgaaaccaaaaagtatgggaaaatactttaattttagagttaacaacatttatagttacaatcatgaagcaggtatggtgctgcttcatgagccaagttcataaaagaaaaaaaaatcagatcttactgagttctgtgagacagatcattatttttcttattaacatattcatttattcagcaaatgtttattaagtgccaattaatgtatcagtggctgctttaggcactggagatatatggtcagtgggataaggggaagtctctgtcttcaatcctcacagatcttattttccataagtaagtgtacaaatacgggctatgaattaaatagagcaagataaagagatgggggtgtggcttcagttgtgcacatggtgcccagaaatctgatagctccatcatggagtccttcacttgagacttcacagcaggatgtaaaaagcaacagccaggaaactccttagttttcccaaagtcctcccttaatctcgtttagagaattatgaaacacttcctatggcttcatcctttatcccttcttcagttgataactgaagggctaaatctgccaagtcttcctttgccaatggttttgtgcgagcagttctccaacagcgcttccactaaaatcttgtcttttacaagatggattttacagttggttggcattgtatgacagtctgtgagtagggacagcccaatgtatgtttgttaatcaagtttatgccttttacattgagtaatttttaattacctcgtgtaatttttaacagcactctccttcatcttcagaagtgttcagtttgaacaataagtttgtcatcttgtcagtcaaaggttgaccaacaaagactgaccgcagtgggcggggtattcagcagggttagatgtttctaagtggtcagtgctttcataaacattttcacgttatgacctttgcctcctcacacaatcttgtaactatggagacttagatgataaagattcaactaagaggatccctttaaagtcttgggtaccagccataccaaaagctgttttatatgaggttctttatgattataatttggacatgaacatggagtggtcctgcttcagcaagaaatcaggctaataaccattagcatccagtgtgggtaaaggcgggggctgccctgaggagtgttgtgtggcacagggcgcatgaacaccagataagtcctcagacagtgcccttagcatccagtggtttttggcatctagcagggagtaaaagtatctcatagattctttaagtgtaaacaccgaggacacacttaacgttttaaaagattgaaatccagacagggctggggtggactctaatctgtttccttctctctctttatgtgttataacttggattcttcattccccaaaatgaccttgtttatataattctccagtcttatctaaaacttttatgtgaggaccatggctcatattccgcagctttccttctctcaaggagctaggactggttattgtgataaatgaaggaaaagaaaagaaagtgggtttctgtttattgtatgaaattgatgtaactttgtatttctgattataaatggtagccagagggaggacacaaaaacttcaaattccaagccccataatatattatggcctgaatagtgttggaatttatttatttatggaatgtcaaaaagaaaatagggccatcaaaaaagcaaacagtggaagtctattttctggcccctgtgagtctggctctaaaaacagtgcaaggggttggggtttggtgggactaggaaagcagatgctcgtcatttaccttcctgtgtttgtcatgagaattgtctgtactgggattgtcaccttggtcagtaattgtttcgtttatagtaccattgtctctccaaagagggctgagttccctaaaaaaaagcaggcagcgcaccacaacatctggctagtagcacgtatattatcggtgatagtaaggacacaattctcgtatttaatcaatttggaggtatgcattttttcgtgggctaatatccctgaaatcatgatgcatcttatggtcagtgacttaaaaagcattatgtcatagttaaattgacagtgtttttcctaaattatacacagaataatgcctttttcagccaatgttattttagagttgatgaaatatgttgcatttttatattcttggtatgaagactattaactgtataataaaattatttaagttctgatgacgtagtgtcagatgatgttcagtgtagcaaaacgcgaggcacaattttttgtaaaaaggacaatatcttgcagtcagtacacactgagtacataaatgttgaccaaatgaagagcacagaatggggctgcccacctggaggcagagccatgaattggtactgaggcaaacaaggagagctttgtgtgtgggtggaggcgcagggagggagcgccaggcttcatggaggcatgcgagggtctccagggtcaaacagacagaacctcagaactggtgtgaagcttagaaggggctgcaggaggctgtgctaaagcctcagtgtattgctatgggactggcaactaaacaagtaaactaaaggggtgaggtacttttccttaagtgaaaaaagtttccttaacttctttaaagtgtaagctctgttttcagcttaactcttgaaatgagaagcgtgaatccgtggtggtttcatgacctgcataatcacactacctgtttttctgtcacagattgccttctctcagcacagcaattttatggacctggtacagttcttcgtgactttcttcaggtaattttgcttatacagactctctctgttcttttgatttaggcactagatccattaatagagtatcagctcctaaaagataaaaagaataatcctgtgtttccaccacagattaaatttacttcatatgtgttcctgaaaagttagatgtcagttacagtaatataaactcaatctagcatgacaaagtgaaaacgtttggtttgtatgttctggcttccagcggggggctttgcatgtatcctgtattaattttgcatgcatcctatagataatttggctcttcatatggggagaatttggactaaaacaacatttaattaataatggttactgatgtgagctacacatatgcttatgcttgttcaagagatgattctggtgattatccaaagttttaaagctgaaagtgattttttagtgccctgggattttcttttatttccatagtttttttcgtagtctaattttatctttatgaaaatactatagacaaataattgaaaaagacaaaaaacgtgctgcagtttttaaaacagaaagcagcaatagcctgccttgcgccttccccaccccacgtcccccaatttaaattgttttgttttttacttctctgtgtttacacttggtttttcagttaagttctcttttgatgtcctactgtagaagatgaggatatactgtgttacatggcctcccctagcatctcccctcacaccacaaaagcatacaaaagccccatctctttcatttaataccattgtatcataatgcatggctaaatcagtgatccctatctatgttatggccataaaactcttattcacagctgcattgaacagtgattacactaccacattttttgggtagaaggatcattctttgcttatcctaaatgtagtatttgccatgttttggctttttaattgtttgatttttctatgtgcctacccctgatttatcactatactcttgctgatgtacctttagaacagtaaaaaataaaataaaataaagtatcattgtactgtgacagaactgagaaatctcacaatggtcacacacaggaaggtgctctcttggtttgaatttctctcggagttgcccaccctagaaccttcccacctcctgcccccaagtgcacttgttcctgaggcctgctgcacaactgtcatttggaaacttcctttgccttcctcctatattgaagtctctgtttcccagatcccatgtcttcaccttcctatccattccctctttttactggagcacattctctagatttatgagaaaagatgcctgggagataacttttctgtgagctctcctgactgaaaatatatttattttatcctacaatttgattgaatttctgggggcaggtgctatggctcacacctgtatttctagcactttgggagaccaaagtgggtgggtcacttgagctcaggagttcgagaccagcctggacaacatggcaaaaccttgtctctacaaaaaatacaaaaattagctgggtgaggtggcgcaagcctgtagtcccagctaatcaggaggctgaggcaggagaatggcttgagcctaggaggcagaggttgcagtgagcggagattgcaccactgcactccatcctgggtgacagagtgagaccctgtctcaaaaaaaaaaaaaaaaaaagtctgactacagaattctaggttcaaaatcattttccattagaatttagaagacgttatactattttttctagtgtccagtataatattgagaaggctagtgctgttataattcccactctttgtatgggatccgttttctctcttaaaaagaaaagtatatttaagtgtaatataaagttacagaaacatttgcaaatcctatgtgtgtagctcagtgaattatcacaaattgaacacttctagagtattgataatattatctcttgttcacccagaacacttcaggcttcttatttcttctctgtcagtttcggcaagttgtatatagttttcttctaatgatcccattccatctgttccattctcttgttcactgagaacaagagttaatattaccaatactctagaagttcctcttgtgtttcccctcactcacaacctcctgcctcttccccaaaggtaccatttacctgacttctcaaaccatacattgcatttgctatttttgaaatgcatatgaattatttctttttttttttttttttttgaaacttagtctctctctgtcacccaggccagagtgcagtgttgtgatcttggctcactgcaagcaaccgccgcctcccgggttcaagcaattctcctgcctcagcctcccaagtagctgggattacaggcacgtgccaccacgcatggctaatttttgtatttttagcagagacggggtttcaccaggttggccaggctggtctcgaactcctgacctcaagtgatccacccgcctctgcctcccaaagtgctgggattacaagcatgaaccaccgcgctcagtcaaaatgtatataaatgaaatcacaacataaattttaagatgatttggttgatgaagaaaatactgaaaacttgtgggttgcagttaaagttgtgcttcgaggttatagctgtaaatctgtggctctccaccttagctgcacctagaatcacttgcggagcttttgatgtccaagccactcttcagaccatttgactcagaacctctaggggtgcaccccaaacatttgaaaactctccaggtgattccaatgtgcagctaaggttgagaatcactgctttaaaagcacatattagaaaagaaaaaacattcctctcaataactcagaactgctaattaaacctaaagaagtagaagaaagaaaaatataaataagagcaaaaattaatgatctaagggaagaagtacagtagagaggatcttaaaaactgtcagttggatctttgaaaagagtaagaataatattgacaaacccctggcaagattaaccaaggaaaaaagagagaaggtatatggtatatgtagccaatgtcagaaataaaaaagggggcattcctccaaatcccgcagacgtttttttcctccagaagggtatttgatgaacaactttatgtgaatgaatttgaaaatgaaacagatggaatgggaccattaaaagaaaaatatgcaacttgccaaaactgacacagaagaagtaaggagcctgaatagttctgtatccatttttaaaacaacactaatttaagttttgtcatagtgaaaactctaggccccaggtggcttcattggtaacataacctttttaggccgggtgcagtggctcacgcctgtaatcccagcactttgggaggccgaggtgggtggatcacgaggtcaggagatcgagaccatcctggctaacacggtgaaaccccatctctactaaaaatacaaaaaattagccaggcgcggtggtgggcgcctataatcccagctactcgggaggctgagacaggagaatggcgtgaacctgggaggcagagcttgctgtgagccgagatcgcgccactgcactccaacctgggcaacagtgcgagactccatctcaaaaaacaaaaaggagaatactgtgatcatcttaatcaatggaggagataaagcatttgataaaatttaatacccattcatttttaaaaaaatttcttagaaaactagaaagagaaacttctggaacactgataatggaacaacataaaaagtcaacagcaaaccactttcttaatggtagaatgttagaagtttgagtttgggaacaagacagagatccactttccacagtttcaattactcagggtcaaccgtggtccaaaaacattaagtggaaaattccagaaataaacaatttataagtcttaaattgtactccattctgagtagtgtgatgaaatgtcgtgccatcctgcttcgtcccacctgggataggaatcatcccttggtccagtgtatccacgcccctatgctacccacccactagtgtcttggttatcagatctaaaaatcatagtatatgtagggttcagtactatccatggtttcacgcatccactgggaggctgggaatgtaaccccacagattaagcaggacactatattgccaaatccaaaggatactttttggttctcctcttcttgacttctcggtgatatcaaccaactgactgcttcatgagcgagactcttccagccgtctctgacccatgctgccctactgctgcttcttagcctgcctattatgcaatcttttctattcagcctctcactgttgattttttcccagaccttgaccctggccgtcttctcactctgtgttctctgactggactctgccttccccatctgcagcacgacatgcccagaagtgaccaattccatccttcctcaccggtgttccccacctcactaactggcaacatggctctgctggttccaagccacttctcttgctttcactcagcaggcactcccttaccaggccccagagctgtggccgtcagtgcactctccacctccactccccaccccaccaaccctagcatcacagtctcgcctggaccttttcacttgactttggcccctccagaatccacaaaaagtccagagaagacctttaaagatcatgtatctagccccttaccttcctgttgtatctaaaataaaattcagcgtcctgggtgaactggcgtccgcttttccatattcagcttgcactcttctcttccacatttctgctaccaggaccatctcctatgtcttcaaaagcacaagctctttccctacactgagcatttgtacatgcttatcccattttctggaacactcttctcactacccttcattctgtatactttcataccttgcttgatttttccagacaacaacaaattctccaattctctgacaccaactcagtgtctcacagttcagttccattcagacactacccatagttggagcaaatcctgcaggttaggggctcagtcccacaagactgctcccacttcagacactaagcacaaatgggtccccaggctacctcactgctactgggccaactacaaattcagggcttctggtgactgctcactccaagatttgacaatttgttagaacagctcacagaactcaggagagcactatacgtagaattacagttttattatacagggtacaactcaggaaccgccaaatggaagacatgaataggacaaggcatgggggtgggtgagcacagctctttcatgccgtctctggacaagcctcctccctgcacgtggatatattcactatcctggaagctcccccaagcctcatcattctgagtttttttattgaggtttcattacataggcatgatggatgaaatcactttctattggtgactgaactcatctccagcccctttccccttcttggaggttgggggatggagctgaaagttctaaccctctaaccacctagttgggttttctggtgaccagcccccgcatctgaggctctttaggcccctctccccatgagtcattcattagcacacaaaagacactcctgtccctctgaaaattccaaggggtaagctaaattgtctccccttgttcctcactcctgtagaactgtattatttttcatcgtggtattcatcacaatttgaagaatatattcatatgtttacttgcttaatatctgggtcggtagcccaagcttcaggggagcagtgcactgtctctcgttcactcctatacacacacagcttcagcatctagtacataatctagtaagatatttgtgggaagtaaacttagactcttcagggtaagtgcagagtgaggaggaaccataggcaccaacctctcagggaaaaaagcttgtgtcaatggcttgcttttaagggatgtaggggctgtattgtagctccagtagacatccttaaattcttggagcatgtgtatggaataagatggccctgctgccaagtacgtacagcctggtgaggaccggccacttctactggacattcagtccctgggcgaccgaatgacactatcagaataagtcgaggaagtacttttcaggcccatggcctcttgtgtagtaaacaaggacagatggcattttcatcacttctcccttgtgggggttgaacttctggaaaagaatccggaactttggtttttctagacgatgtttactaactagcgatgataaacttttagctaagaatagagttactttcatggcatggggaagaaaatattttcccaggacaagttaaccatgtctaaaagaatttatttagctttttttttttttttttttttttttttgggatagggtctctgttgcccaggctggaacacagtggtgcagtcatagcccactgcagcctcaaactcctgagctggtctcaagcagtcctcctcacctcagtctcccaagtagagctgggactgcaggcatgcaccatcatgcccagctaatattttcttttttgtagagttgggttctcactatattgcccaggctggtcttaaacttctggcctcaagcagtcctcccatctcagcctcccaaggtgctgggattaccagcgtgagccactgctcccagcctggtccaaaagaatttaaaatggagcctataaggaaaaggggaaaggtatccagataatcctaagcactctgaagaatggctcatataatatatggaaaaaagaacaaaagtcggggggaaattcgatgattcaaatgagaaaatcaatgtaaagtgagttagaaaaaatatttccagactccgggcacagaaaagtaaactctaggtcctgacacagtctgttgagactgtgtcctaatgagatgcttttccagcctcaggaaggagcctccttaaagagggaaataagagactctcctctgcttgcttttctgtagcttcttgtagctggagcagcaagtagaagggactggggaactggtgttgtcaacagatcttcactgaatacagaagggccctctcaccttccctgaggcatagagggaagcaggcacagactgtattacgggtggacccaagagaggtttctgagtgacaccagggtcctcgcctctcagcagcagcggcaggctcttgaccaacttagaagctgttgccgttggcttcatgctcttccttgtggatttgctgcctccttatgtttatctattagaataaaaataacaaaaatcaggattatgcgtggaagccaagaaagactgagaaggttattctcagacctctttactggttcttcctgttctcctcaacttctcattgttagggtggccccaggacattctcatctatctgtattcactctcttggtgaatgtggccaggtttattgcttgaaataccatatatatccttatgccttccaaatgccatgcccagcccagacctggcagccagcctccaggttagaatatctgcccaggacatctcccctgggctgtccggtagatatctcagggttctgcatgtctaaaatggatctactgatatgtccagattccaaagtgcttttcacacctctgcttccacagctctggtttgagccaccatcatctcacctagactgctggtctccctgataatgccattgcacctctaatggttttctcagcaaacgttaaatcacaacactctgtttaaaaacctgcactggctctttcccaatgccctcataatgcttgcagtgccccacatgatctgcccttcaccctcacccttctctctgcctttgtctctggccttacacaccccactccaaacatgctggcctcctgaggctccccaagccatcccaggcacggccttgcctcgggctttgcactgactgctcctttttccagacgcacttttgcaggcgtccatgagccacctcccataccttctccaagtctttgttcaaatgtcaccttctgagaaagaaaggcccacacactctgaccatcctgctgtggagtgccactggtactcctgaaccccttgctcttagcatctttttatttctgtagcacttacctaccttttggcatgctctgtaatttgcttatttacttactgtttgtctccacctgctggaatgtaaattccatgggacagtaatctttgctctgtttatcagtgttttccatgagcctagaacaggacctgacccaattaaagtattgattaaaaagacaatgtaggccagtcacgtgaggcctgtaatctttgccaaggcaggcagatcacttgaggttaggagtttgagaccagactggccaacgtggtgaaacctcgtctccactaaaaatacaaaaattagctgggtgtgatggcgcacacctgtagtccaagctactcaggaggctaaaagaggagaatagaatcgcttgaacctgggaggcagaggttgcagtgagctgagatcgtgccactgcactccagcctgggtgacagagcgagactgtgtctcaaaaaaaagacaatgtagactgtcagaagagttggcagtgtcctgatagcagagaggcccagagatgaaggctttccaccaagtgacgatcctgggggggttggaaggcaatcctattgaattgcctgttgtcagggcacccattggaggagtctgcacccttgtaccccttagggaggaggcagtgagccttgagataagggcagttcagtcatcctagcatcttagagtccctctctccagccagcccaggagggctgtgttggtgtcagcagttgggtgcactgaggtgccattcatgaagctgaccccatcctacaaacgcagagctcagccagcaagagagtaccttgatgacactggcagagagccttatgtctacatgtagcactctggaaaaataccctgtagctagccccttttacaagttaatgagtaaaaatgcagttatagcagttatctatttttttacttgttaagcttattcttccttttaaccgcaagtcagctgttctctttgccatcactgtagcttttcaaatcattccacacctggtttatacatgagagagcattgaaatcctctgcaactattaaggccactgggagttattttctcttaatatttttaagaaagttgttggcttgggtttgatcatttcaaatgtgttttattttacttaaaaatatgtatttgtaaaaatattgaatatacactaataattacacacatttcttctgtttgcctcttccttatgaaaagttgattgttactttaataattaactttttcaccttcttagattttaccagtaaatatctgctaacttgaataaacagcatgactaatatttgttataattatcttagaattatttttcttatttgggataagccactgtgggccattctgatgaaggccaagtggaaggtcagggctctctctgaataccacatgggaacgatacagtctagaggcctggttaggaagcctggcttaccgaagttgtctcactcattacctcatgagccggcacattgttgtgacaaagcatcagaagggagcccggaggtggcatgtgggagctgttttcactgcctgggcctgaggtgtgggcaacggtgggagctgattctgggggctgggataataactcagtactttcctttctcacagttgtttcctctctttgctcctggtggctgctgtggtttggaagatcaaacaaagttgttgggcctccagacgtagagaggtaagcttcagtgggtaaagattaaagaatccctggaagagctttttttccttctttttctcttaagcaagtgggttttagctatttagtgataatggacagacagacatctccacggaaggaataatgcagccctttctgcagtccatggttctgcagcatatggtcagtagacttagatgagtgctacataaagtgtaattagttctgcaccacagtgtcttaccttccgaaaaggaaactctggtattgacttggaaatgccagttttatccttcagaccagaacccaagtcctgactgaggccagagatccaagggcgtattagcacagccaccgcgtgattaggcaccgccttcatgagaacttgtcatgcgagagggaaatcagccatttaagcatctcaaaaatttttcattattcaaggaaagataaatgtgtgtgtagttaagtttttaacttgagttttatttttaaataagtcttgatttgtttccagagcttcattccaaaagtaataagcaaatatttacagctgacaaatttaaataaataaacttggttgcaaattacaacatatcaaatgcccatcaatcaatgagtggataaagaaaatgtggggtgtgtatgtgcgtgtgtatgtatatatatatatttatatacatttgcagcaacctggatggaattggagaccgttattctaagtgaaataattcaggaatggaaaaccacacttcatatattctcacttacaagtgggaactaagccctgaggatgcaaaggcataagaatgatacagtggattttgaggactcgggggaaagagtgggagggggatgagggataaaatactatacatcgggtacagtgtacactgctcgggtgataggtgcaccagaatctcagaaatcaccatgaaagaacttattcatgtaaccaaacaccacctgttcccccaaaaacctattgaaataataaataaataaataaataaataaataaataaatattttaaaaataaccaccattaaaataattacaacataaaaactttgtgtattgcaactttcacttgcatcattactttgcctttcatttagtcatgcctgtggcattgttggcattgttgctaatggaaatgctcacgtgtatataagatctagacaaagaagctgtctggggtttttttggatctactcatgcctttttctatataaaaatgtatttatagataagatttagaacagaagggaagtatgtaaaatcacaactcacaagcactcagcactgttgataggatattcatgtctgaatagggttaagacaggctccaggatatgggcctcttgttgtgggccacagtaccaccttttctgacccacataaagatgatgttatcatagagggaatgttgcacactctttattattattttttaaataagtggataacatattcaaagggttttttagataactgtattactgatatactttaaaagcctaatgaattacgtgatgttctgaaaaaaattatattttcaatttaatttaaatgtatatttattcacaatggagtaagaaagaagtaagaagccacagtaaaccagttatcaagaaaggagttgaaaacttttgagtgctactctctccaagaaggacattagacccagggagtttcaaggttgagatgagcttatggagattatgatagcctgttcctccctgagtgcttatgagaaataattgtttctaaaatctgtaaagcaaaaggacagatggaaagcttgaaagagtccgaattgattttatacacataaatcttggtaccaaaacctgacaaaggtagcaccaaaatacaaagggcagctgcatagtgaatgtatgtgcagcagtctaattgcagtgtaatgaaagagtcacttaataccagaaaacctattagaatatatcatgtcaaaaaaagaatatatcatgtcaatggggaaaaaataacacctgaattcttcctagtggcccagaaatcatttcgtcatttattgaaactcaatatccattcctaatttgaaaacaaaactcacagtaaaatcaaaaatacctttttaacgctatgaagaatatctggataaccagcatcactcatgataaaacaaacaaacaccagaggttgtatccaggctagttagttttaacaagagctgacatctgagcatttaaaaattccaggcactgagttctaagtggttttacgtagattaactaatccttatctcaaacctatgaaataggtattgtccctcgttatgttttatggatgaggggactgaggctcagaaaggtgaagtgacttacaccacatagttaactaagaggcagacgaggctttaactagggacttctgtcttcacagcccaagttcgttatcttcgtgtaatcctacagtcattcagctttgtgctagatgttctagactgtgcactaagtgaaagggcactaagaagtgttgttactattggaaaggaaacaaaattattattttcatataaagttattaaaacaatgtagtagagatttgcctgcataaaaatgaaggcttaagtatatcaaaaaataaatgaataacacagaagctaaacaatcaactggaaattgtttgcagtgtgcatgagagaaagtaagatgcacagcctacaggaaaaagattaaaatatatatatgtgtttagtatggagttggtcatctaataaactatgcttttacccgtgtgatttggaatttctctgtaactgtgaagctgaccattcagaatatatttcatcatttttaaaatgtcagccattatttattccctctcagaattgtagaagcaatgatttgatgttatttttaaaaacaatagtagtagcggctgacgctgtttagtccttgctacacaccagccccggttttaagtgcttttacacatgttgcctcctgaaccctgtcttattgtgaccccagcacttaagcacagaccccaaggagcattgctgcatatattagtcgagtaaatggacctacagctttaaaaaatgtaagaatgttggtattttaagaaaatatttataaaaatgcccttctagaacagagccatatctatccaggccaagattctgtcttctacagtagttccaaggaggcagtttgaagtaaggcctacttgtatcccaaggcataacctcaaagcttcagcatcatgcccagaaagatctctaatatccttaatgatatgtgaacggctgttcaccatgtgctaggcgctgtgctgtagtatctcatttgatcccagtgacaaccttatgggctaggaacacatttcagagggaggacacggatgcccagagacttcaaacacctttcttaaggtcacacagctgtagactattggagcaggagttcacatcaagcctccctaaatcccgagcccctgtttgtccctctactctgcaaaatttcacatttgtttgtttttagcttataccacctctctgaaggaagggttctttcagttcctatctgccgtataatgaggcataggtttttgttgtcctgtgaatccttggtgccagtttcagtaggaagaaagcaagcctgactttagtagtagaaatacgaggagatagggccacaccattttatgtatttgacaagtcgacgtcatagcattaaatccacagtacttttatttttatctttattttcttattttactttaagttctgggatacatgtacagaacatgtagatttgttacataggtatacatgtgccgtggtggtttgccgcacctatcagcctgttatctaggttttaagccctgcatgcatttggtatttgtcctaatgctctccctccccttgctccccacccctgacaggccccagtgtgtgatgttcccttccctgtgtccatgtgttctcatggttcaactcccacttaagagtgaggacatggagtatttggttttctgttcctgtgttggtttgctgagactgatggcttccagcttcatccgtgtccctgcaaaggatatgaactcatttattttatggccgcatagtattccatggtgtatatgtgccatgttttctttatccagtctatcattgatgggcatttgggttggttctaagtcttcgctactgtaaatagtgctgcaataaacatcgtacgttaatatgtatctctgacaggggcttttaaaaaatataaggacaatggtgttatcacatccaacaaaatttagtaatccctaatgttatctaatatcaagtctaaaaagtatcattttggacttgatttgtttgaatcagggaggatgaggtgtttttaacatgtagcattaactaataaaaccccttcagtactcattccattaatggctttgccatattatttatcagcaacttcttcgagagatgcaacagatggccagccgtccctttgcctctgtaaatgtcgccttggaaacagatgaggagcctcctgatcttattggggggagtataaaggtgagaatgtgactcagaagtccctataacttgactttttaaaacttaggctcctaagtctgggaaaccagagagagcaaaagccctattccattacctcctttttttcctctctttctttttaacaataagctaaaacctgaagttgctgggtactcttttgcttttgtttttcaatctttgtttcatgtcgctgagcagtcttgtgttcaggggattttaaaatctaaatgatttgtctgcttttcttttaagtattctgtggcatacaaaacttttatttgaaaagagctacattgaacatcgaatcattgtttttttgtttgtttgtttgtttgagacagagtcttactgtgtcacacaggctggagcgcagtggcatgatctcagctcactgcaatctacctcccgggttcaagcgattctcttgccccagcctcctgagttgctgggattaccgatgtgcgcaaccacgcccagctgatttttgtatttttagtggagatggggtttcaccatgttggccaggctggtctcgaactcctgacttcaagcaatccacccacctcggcctcccaaagtgctgggattacaggtgtgaaccaccgcacccagcctatttttctttatattttggggaattttttaagcagcaaaatgataatacactgtgtattaatactctgtggaaaatatttatcacccccctccccaaaatgatgaagaaaatggtaagactttctccttgggcaaaatgatggaatatttaagatacgctggagaaatagctatgtatcttgaataaaacatacttacttaaaatgttactgagctcacaggaagtaggtaaaatctccagggaatactctccttccctaccctaaaaagaacaaaccataaaccaaaaccagaaccatgatttgtcctaagtggttccagatggtaaacacaacgcccacctgaatagatggaaatcctctctatagaaaaacatcttcaattcagcgctgttgaagtcccacagatcaagatgaagtaattaaagatcaccagggacaagaggcaagccgccataaataagagtagcagaaacaacagatgattgatgtggacaggactccaaatgttagaattgaaatctaaagaatataatataactgtgtatgaaatgtttaaagaaataaaagatgaaatcataaagatgagagtcaagtatggccaagcagatctgaagaagaaccaaatggaacttctagaaatgaaaaatatacttgttgaaattttttaaataaataaactcaatttacatgttaaacagcatatcagacataagagaatttacaaagtggaagatgaatctgaaaaaagtacgcagaatgcagctcagagagacaaggagatagaaagtacaatagatactaagaatatggaggatagaatgagaagatccaactcatatatatttccagaatcccagaacaaaggagaggcaatattcagaaatgatagctttccagaactgatggaaaacatgaatatacagattccagaagcaaaacatattgtaagcatgattaaagaaagaaagggaaggcaggaaggaaagaaaacaagttggatttctcataatgaaattgtataactccaaagacaaagagaacatcttaaaatcagccagagaaaaagtatcacatacaacgtggagaaactgaacgttagtgcactgttgatggaagtataaagtgatggagccactgtggaaaacagtaccactattccccccagaattaaaagtagaattatatatgatctggcaatctgggtttatacatagaagaactgaaagcagagtctcaaagacatatttgaacatcagtgttcttggcagcattattcacagtagccaaaaagtggaagcaggcccaagtgtccatggatggataaatggataagcacaacgtggtctatgcatccaatggaatgttatccagccttccaaaggaagcaaattctgacccatgctacaatgtgatgaaccttgaagacatactaagtgaaatactccagtaacaaaaaaataaacattgtatgattacacttttaagagatacacttgtggtagtcaagctcataaagacaggaagtagaacagtggttcacaggggctgggagaaggggaaaatggggagttagtgtttaatgggtacgaagtttgagttttacaagatgaaaagagttctggagatggatagtaatgatggttgcacaacaatgtgaatatacttaataccactgaactgtacatttttaaatggtcaagatggtaaatgttatgtgtattttatcacaatttttgttaaaaatgggaaaagaggccgggtgtggtggctaacacctgtaatccgagcactttgggaggccgaggcgggcggatcacttgaggtcaggagttcaagaccagcctggccaacatggtgaaaccccatctctactaaaaatacagaaattagccaggcatgatggcacacatctgtaatcccagctacttgggaggctgaggcatgagaatcatttgaacctagaaggcagagttcacagtgagctgagattgcaccactgtactccagcctgggcgacagaacaagactctgtcgcaaaaaaaaaaaaaaaaaaaaaaaaaagggaaaagatcacctgcaaaggaatgacaagctgaccgctaacttctcagaagtcagaagagagtgagagaatgtctttgaagtactgaaagaaacctccccacctagaatagtgtgcccagtaagcaccttccgagaacaaggatgaaataaagatatcttcaaattagaataaaaattgagaatttgccaccagcagacctgcacaaaagaaatgtctgaaggatgtgctaaaatgaaggaaaattacccccaggaggatagactaaagtgtaagaaagaatagtgagtaaataaaagtataatcatatatagaaatctaaacaaacattatcctggtaaaataatatttgtgggttaaaaaatagacaagcctaaagtattgaacaacatgatatatgtcagcagcatatgacacagttagttgctccttaaaacattttcttgattttcttccaggacaccacacacttgccttctagctgttcttgctcagtggtctttgctcagtcctcttcatcttttgacagtggtgtgcgtcaggccctgcctttgtccccttcttgtgtcttttacacccacttcttcagtgatcttcactatcacaggtttaagtaccatgatcctctcacctggactgctcccatgagcctccaacacaaacatcagttgcctactcagtgtccccacttggatatcttaacatggccatggccaaactgagctttggatgtttatttctcagacttgctcctctgacataccttccgcctttcagcacataacacctccatcaccctagttgctcaggccaaaatcgtggaattgttcttgactcattcttgatgtcatgttctcattggacagccagtctatcagcaaatcttgactccgtattcaaaaatgtttcccagccactgtgtcccatctctgccactattcccacttgatctaggccaccgctgtctctcacctggattcttgcagttacttcataactgatctccctgcctctgccattcacccatctagtcagttccttgtgccacagcagagtggtactggaaattattttcttcagccaaactgaagaaactgatgacatttgtgggtatagtcagcagaattctaaaggtggccctcaagatttccatcccacgtttattcagtcacacactactctaggtactgctagagggattttgcagatgtaatgaaacaagggagttgactttaagatacagaaagtttctctggctggtggcagaaagaggaagccagagagattggaaacatgagaaggacttgacacaccatccctggtttagagatggagtggccacagaagtttttttgtttttgtgagacagagtctggctgtgtcacccacactggagtgcagtggcatgatcatggctcactgtagcctcaaactcttgggctcaagggatcctccctcctgggtctcccaaagtgctgggattacaggtgtgagccactgcgtaagccctgaatgcatttcaaagcagagtcttccccagagtctccaactaagagtcccatccagctgacaacttgacttcagcctgagagacctcaggagatagctgataattccccagaactcatggaagacatgaacagacctgtctctctcggcagagaaccctacagagcttgctgggacttctgacctctaaagctgtgagataataagtgggtgttgttttaagcaactaagtttgcagtagtttgttacactaatataagctgggtgcggtaggtcacacctttaatcccagcactttgggaggctgaggtgggaggatcgcttgagcccaggagttcaagacctgcctggacaacatagtgagaccccatttctattctttaaaaaaaaaaaggaaaggaaagaaaaagagaagaaactaatataggggacaaatctcatctaaaggctgcaacagaagatgaaaacatattttccagagccaaatgcatccagagagtagacaaagaatctacttggtcagccaacccagcttttacaagcttttacatccagcaggcttgtctacttagtgtctaaaggacagacaaaatagaatgttttttaagtctccatgatatctgcatctgtgtttgagccaggtatgcagaactatctgacaaatctaccaagcactcaccactgccattcctgtgacctacagttatgaattagaatttcaaatcgagccaaagtacagcatccagaagaaacccacttggcgtggccccttccctacagttcaagcagccctctggaaggggactgcgagacctcagtggaaggtagcaagcagtaccacccgtgtcactggtgctcagagagtactgttagctctctcctgccctttttcattatcttaaaaactgcaatgtaccttttttatatacctgaaattgatggttcttataagcacaaatgaccgttaagttgtccgtatacattaaagcttgagtatcacttaaggtaattttgttttcttctgccagactgttcccaaacccattgcactggagccgtgttttggcaacaaagccgctgtcctctctgtgtttgtgaggctccctcgaggcctgggtggcatccctcctcctgggcagtcaggtgagtagatgcggtccagcgaaagacaccttctaagcatgtgagggagctaagcatgggatacttccctttcctagagaacaaggttataaaggtgataaccaacagtgcctgcccatgccatggcaaggattcagtgagataacccattttcacctatatgcccaaaactgttttcccgaggtcatcgtcccttcctggggggacgtcccgggacctcctcaccgttctggcctccccagcctctcagtgctcgtggatgcctctgaccacaccttcctttgctgtcggagtttctggtccccctttgcctctgctgctctttccctgtctcttttgccctgtcctgcccctgaatctgggtgtgctcccttcaccctgcatgttcttcctcagttgccttctccactccacaacttccagccatctcccctgggctggtgtttcttcaattggtatttctagccctcctgcatttcttttgctttttcactgttttaaatataaggcacaaacacaacgataacataaaggaaacgtctgaaaatccacgcacagttctgctatcccacaacccaccaattttcattgttctgtgttctttccagccctggccagcaatttagttgaaatgattgtatagatctaatttttattctgcttttttcatataactatcttaaacatttttatagcctactacataatattttaccaatgcatgtgacacttctccatcaaatatatgaatcatgattatataaacttctattttgaagacagaaaatatggaatacagttttgaaagtatctcgcttaattacagagtgctttggaggatgcctcaatgtgatcattttagacaactttcagttgagatgattctggagtcagggttaccttagccagagagcaccctgtgtctggtggaaaggctgccccaggatactggtccaggccctgagctccactgtcccttcagtgcaccctcagacccagaagatcctccagcccagctttagaatggttaggatctatagtatcttcacgagggaggcctttcgacatggctcggccaggattcattgtcttcatctcacctagggattcctgtgagttgagcccctaactcggccttgcctgtgttgaggacaggacaactctctcgtctgtcactcaagtgcagtttgtgtctcctgccaccgcccagcccagcaaacagtggctggtgtgccctgagggcagggtggcggctggggcggcaaggtgacaggtgtgcagccctgtagcctttcctgctccagcctttgcctgacatgccccaaccacctgaaacacctaataaaggtctgctcaatgcttgagacccacccaacagtggggccgtccttgctgccccttctgtaccccctgttttaagtggtgcccttttctaggctcccagagcattccccactgtgtgccaggcacttacagctgtctacccccacccccactccccttcacaggctcaacaaggcaagtaacacgctccactaacacaactcctggcaggtctcagcagagctccgagcctgcgctggccatttcccacgtatattaggaatccagctcccccagaaccgtgccttccctaatggatgtcagattactccgttatcaatacaggaaggaaaatgggattgcaagtccccaaagcaaagtgggcggcggggcagtgatgagcataaagggtagttgctattgatcgtgggttgctgcgcgccagatactaagactttatgcatgccaggtactaagactttgtcagttcactcattgattcttcacactagccctgtactactattatccccattttacaaatgaggaaactgaggtgcagggaggttaaagaaccttgcttattccaaagcctggccccagagcctgccctgcctgtatcgtggtatggaatggaagtgactaatgctgactgagtgataggggtgaatctgaatctcgtcataaagcgtgctgctatttctcggctccttttggaggaaggcaggagaaattgggcacttagcacacgtctacaatctcatttatatgttcccaccaccaagcagagcactgtgcttggttgatgtgctgaaaagcaaactgcaccttttgtgtaaaaagtagggaaaggagaagaatgtgtatttgtatttgcataagtataccctcaaagaatacataaaaattagtgaaagcgtttacccagatggaggggaaggcggagggagggtcagggtggaaggaagacttttccctgtgtatgtgtacctttttgggggttttttgttctggttttttggtttgtttgttttgcaccatgggattgaaaaataaatgtttaaattattttttaaaacaaaataaaagccagctgctcccagcatatgttctctctgtggttctcccaaggtcttgctgtggccagcgccctggtggacatttctcagcagatgccgatagtgtacaaggagaagtcaggagccgtgagaaaccggaagcagcagccccctgcacagcctgggacctgcatctgatgctggggccagggactctcccacgcacgagctagtgagtggcacaccagagccatctgcagggaagggcgtggcggggaaatggctgtgcggtgcgggacggaagactggaaaccctcaaagcatctgactcacctgcatgatcacaagctttctttgacggtttctcccatccgtgttccagcatctaaccttttacttttgcataggaaatacttgatttaattacaggtccagggatgagctgatggttgctggaggaggccagtgtagagccagtgagagaactaggaatgacactcaggttcactgtggaaaactgttcttgggactgtctcaactgtgcaaaaaacaaaagatggagtgtttacaagtagacattcgtcatcagttgttcttgaacatggtcttttaaaaactagtcagatgaattaacttgttttcatctgaagcctgctatcttttttaaaagatgtgctatttattcttgcacgatttaggcaattatctctcttccagggagtacctttttttctagttgagaattaataatggtccatctcttttgatcatatcaagctaggatagaaggggggctattttaaatgtcaaggtcagcagtgttactttgaatgtaaactggtataataggtagttttctatagtaacttgattaatttagtcttaatccatttgaaactctctcttcctttctctctgcctgtccctctccttctccatctcaccctccctctctcacacatacacacacaaacacatacacacaacactaagtgcctagactttaaatagatctagcaattggaaagttagtaagcctaagtttttacataattgcattcctacattcttgtaaaatttaaatagctaccattggcaatctgctttttttctaaaatctgatttgcagccaggaaagaattttctcacccaaggaacatttgatctagcagcagggatgagaggaaagcagaaatgaatgaactgtgaaagctcctgtttttattatcaaaaaggacactgtcaagaaggcgccccctgcccccacccccgtgtcaccctaggcctgataagcgatcagaggaaaggactcattcatgtcacgcttccttgagcagaaaagagcactgagagcacttgggacccctggatcagagagcatctgtgtgtcctgcagcctcctctgaacttgtggttcattctcaggctggggtggactcagatgccaggaaagggacagcctcccattgtcaggcagaagctgcccaaagcctggagaaggacttgtttgccctctttcccccaggaggggctcgacccacccaccctccctctcagaccaaggtggtggctgtgaggagggcagcaaatgctgacaaggatgaaaagcacatggaaaaaaatggacgaggagggaaaactctgccaaatggaaaatgaccaaatttaagagggtgggacagtcccctgctcctctcccagagggcactgcttggaaattgtgttttccccatttatggtgctctgtattctggcattatgcagcagcctcccagaagctctcttctgcttcaaaacctgggatctctggcattaccctattgggatggaccgctggacagcaatgctcgagtttgtgaatttggagagatactcaaaagagctaaaactgcagcattttacctttaaatgcagtgcctagagagagagtattgtctcttccccaacactaaccccactcccatgaagaattgcctggaaagatgttttcaaggaatttgaaccataaaacactatctgatgcacagaacacctctactttgagactcacctctcataaagcttctttttcacattactgttaaagaccagacgttctagaaaagacccctcctctcatgagctcccccatccctgctacagaacacagcacccatggcgcctgcagtggactggccccttaattcccacaggcccccccagcaaggccaaagggaggcccctgggtattgtcctcctacaaggaagatcctctttgtttgttcaaaggaccagttttcctaggccaaagaagtctcttccccatgttagtcctatgccttgaaatatcatgcaccatgacccacagccatctggttatgtcttatttttttcctaaaagataatgtttatttttaaaaaggaaggaaggagcaagtgaagtttcattctgctccagcggtggggaagccgctgaatccacctgcttctcctttgcaaccgacagcaaacagctttctccggcctcagggcagaaaaagggaatggcagggagtaagaggcgctgggctcggagcctgtttccaagaaggaattggttgtcatctggcagtgttgcgcgtcacaagagagcctgtatataaattaaaatagtcaagacaacactgaccttgcacttgtacataactatacagtagtgtccagaatgttcagacattcggagtgtacataaaacagaaaaaatcttcatgtatttttattaaatataacaatgtctgagtttcacctaagatgtttttgtgccatatgctggatatccaggttctcgccaggccccgatacatgaataacaaacccaagaaacgcatccccattgtgtgatgtgttcagatgcatctggcaccaattaggtatttcttaaaacaggactcatctgtcagagtgcacatgaaaaatcaggcagggaatcgaaacgacagcgctggaggagactcaggaagcagaggcgtccctgccgctgcccttggccctgcaagcacatcatgaccctttctggcagcctcttggtgctctgggtagtgagggatgaccagtcttgtcctgagaaatgtttctcttagtctttaagttcaaagactaacctgtagcaatcagactttccaaaagggggttctccattttttgtagttttgtctaaatttttaatgaccatttcctggaatcagtttattatactgaaaactgggggtgggagtagggagctagtttgttgataaatagttcccatttccccgtggagaatttgacataccctggactcctgtgtgcctcctgccatccctgcacacagcctggggagaagcctgtgcctccccgtgtggagagaaggcaaccccagatcccctgagctaacccggaggaaaggcagtcctggacagaagactgtcagcagaaggaaagtactggactacccgtgggtaagtcctgccattcaagactggagacacctgggaaataaaaagagcagggcactgctggtgggaagaggcattttaccttccagtgcaaatcctgctcctttgatttaatggggtgtactggggccaggggctgattcacttccttgggagatggtggtgttttcatgaacatctttgatccttccatttcatttattcatccatccattcaacaagtatttgctaaacactaacttaagctaatgctagggtagtgactgagatgtaaaaatagattttagaattaaaacaaaatccaagtcctcacacccctgtcatcccaggagatctttccttgtggtggtttctgtgagaattggccatcctgaggacacagccaggacggcagaggcctcctggcctcagggcatgccctgcctaccttctgaaatgtttaccccattgaccaaacttggctccagccattgcggtggtttctagatagccaggcccaccaagagatattgccccttgatgagagtcaaacaccctgcctacaaggagatgttttgaaatggagaggaaaattggcacctcatcttttaaaggcagtaatggaattgattttcagtaactgaatttgtgcacaaaacattctaaacactagtgaagcctgtttcgttgaactaattaattctggctctggaaatgtttttgttttatagttatttacgatttcgtttgtttggattcaagcttagtttgttaatatgtataatttagcatctattacactcatgtaaatatggagtaagtattgtaaactatttcattgcggggattgtgggtgttatacatacatttaggactgcaattttttggtatttttttgtattgtaaaataacagctaatttaagcaggaacaagagaactaagggaggtctgtgcattttaaacacaaatgtgaagaacttgtatataaacaaaagtaaatactataatacaaacttccttctgaaataaaagtagatctggtaaaaatgtggcttttgttctgagtgtttcattttgattttgcattgttttgcctatatttacattttggtcattagaaccttaagggaaaaaaaaaagcgtttaccagttttcagactgcgtcagagggagcacttgacagttaacggaggtgagggtgaaaacccctggcaggtacctcagacagccctccaggcagcttgtgagccacagaccttccacacagcctttgtgtgcctacagggcagctgggcccgtgggggcagggacagatctgagcagtgaggtatggacagccgtctcactgctccactaaccacaccactgtctccagtggtgctgttatccctccactttgcgctctcctcacaggctcctgaatgtcctctaggttgtctccaaactgctaaatctcaggtgtgctgacaggtctcagccgtggccagtgttttcctcggccttgaagttttgaggaagttactcccattggcaaggccttgtttgggggcctcctgtgtgtcctgcccccttttatttggctcctgccctcagaatgggtcagagtcatgtatttcagtgcagagtgcagattcccagtgctagcagttccttccagaaggatgaggtcattgaaagaaggaggaaacacaagacggtcactcctgtaggggaagatggcagggtactagggccagggcctgaggtgaaggcaggagcctggaggaggcctgcctaccagagctcaggtctggcttagggagggaaatggctggactagtggctgcaaacaagttgagaaggaccttgaaactcttccagggagggttatgagagccaccaacagtaacaggatagaaaccctgtgatttggtggtactgtggcagagacaatgctgtgtttaccaaacctactgggcccacagctcatcatttcccagcctccctgcagttaggttgggaccaagctaagccatggccaaggaaaggttggaagaagtgatgggcaccaattccaaacgtggcacacaaatgctcccacaggggactctcctacggatcacatgttaaagactggagggactgtgggtccctgagtcaccccttagaggccggccgcctgatcagtaagagctgtgctggcctttgcatcactggaaagcaaactcactctgctgatccactgagatttggggctttgttacactagttagcttttcttattctcactaatatacaaatcagcactttgaagtgcagtgcacatgtaaccaaacccttaagtacatggcactggctcagcgtgtggccaggtggcgagcagtgataagacaggtgcagctggcaggacaactgaggtccctgccaggcggagcaaaacatctggtaaggccgtttcactcgataactcggaaaacaaactccatgtctgctgactttttgacttgaggggaataaacaacaacaacaacaacaaatatgtgttgattgttactggctgcttttggcgaggcatcacgagaaaaagtttacttcaagaaagaattagccagtttacaagcagaagggaaaaagatggaaagatgttaaccagggccttgcaattctggaaaagccaactgcttctagtctccaaccagtaagaggtgcaggtgagcaacaaaggccccatgagacttagttgaacacagtgactgggcctcatggtgaccatccaatgaagggtgtggtcttcccactggagcatggaaactgcaaagccaccagcttgagggaggggcacgtggttaaggaagcaaagaaaaagctgacatgagaactgtttccaggaacaaaccatgaatgtagtcactgaccctggacctgcctggaaataaataaagatgtagtaaatttaagaaaccattagcaaagccttaaaaaaaaaaaaaaaaaaaaaaaaaaacggtggcaattgaggaagtcttaaatccaccttcaggcaggaaaatggttgtgaaagctgtgtccagcgagggctttacaagctcagatgtgggcccaagagcagaactgggcctgagcgaggactccccccagcccaggacagggcccagcaggattgcaaaaacaccgcagagcagcaattgctgcatctgcacccgctccttcctgggtaggagggcctaggagggtgtcctgcctgttctcatcactacctgctgggtgggaggtggattaagctgtcttgttaattcaggattgtggacttcaaagagccacctgagggaggctgcatcacctggaggtctgggcggggtgtgacctcgtattgtccccctaggagagggtgcctgtgacccatgtaggagaggaagcacaaaactggttttggtgatcagaatagcaaactgggccagatgcttaccacacatttcctcttcctgagtccacagcttcgactgcatggcctgggcccgtggcatctacccagatgccttgggaccagccctcaccaatgatgtgtcatttcaaggccattcacagtgacagtgtctcctccactgtctccatcaaccttgaaggcttatggtgaagatggtaatacaacaggattgaaggagactgggtccctgaatgactttgtggaacagagcactataggccaaaggtagtagaattaggtatcttgttttatgctgcttaagtgaattaattaattctctagatttattcacctagttattcaacaaatttttattgagcacatcagggctagggcattgtgtcaggatcagagattttgcaatgaacaaaacagaaaactccatgtcctccaggggagttgataataaaatgtcagataagctctgtgaagaaaggtaaaagtagggttaggggtagaaagtgctggggggattgctacttaacatagtgtggccaggggaagccattctgctaaggtgacagctgagcagaacgatgagtggagggggcaggaaggaatgtggaggtctgggagaagagcattccaaacagggatggcaggtgcagaggccctgagggggcagcatgcctggtaggcctgctggatatttctgagaatgaaaaagtccattcccagttgtttgcgtggagaacctaacccagttataaacacaaagtacttgagagctggtttaatatacagccagctttccagcagtgcaactactgtgtacatcaagggaaaactgaacttcgttttccttaaaacttatcatcagctggtcatcattttgacaaattctgtcaacaacagcagtgtcattcctggcatctgtatgggtcacgtctgaacagacacacgccctgcagccctgcaggtaccagctgtataacaagaactcccttccaccctgtgtcctggaaacaagaaagccattagaccggaagatcccgatggctatctcaaatgtgctggatggagttgccagggcccactggcatgccctgtaagcctttccttccacgtttggttcctgccccttgaagactccatttctgagtttgtgtgtgttttactttctagtgtgtgtcctcatcttaatttttctctctctcttctgccttgaactgaaggttcgcttgggtgtggagagacaggcccccagcagagcagcttcccgagacatcctccgatccagggcttcccagcagcccggcaaggcagggctgtgcctttctgcttcagctcacaagcatgccaggctcactggcaagctgctgtctggttgagggactgctcctaaagccctgcacagcccctgtcctcctggccctctggaaattccacccccgtgtccacatttcatgcaaaaatgagctggttctgtgagcatggcccggcctgactcgcttagtgggcggtaagtggtttccacttcaaccttgcacctaatcaccgggctccacaccaggatggacattcatgagccgtgaagtttccagtaataaatccacagatgcttccagcacctgccttttcgcatcacctccactcccagccacctgccaggcaacaggtaacagagacccagtcacaggagggcagtgtgggggcaggactgcagtctcccaaagcccatgcacaaaaccgacagcgccctggcaggacaaggaggctgacattcagatgtggaggaacaaggcatgacccattcctggtcatgggggccacagctggactcagccttgaggcttggccagacttaacaccgtgtataaaccaggacctttttaggtagagtaatggaaaccaaactctaatgatcttagacagtgctattagtctcctggagctgccagaacaaattaccacaacttcagtgtcttgaaacaagagaaactgattctcacagttctggagaccagaagtctgaaatgcaggtgttgccagggctgtagtctctggagactctaggggaatctgtgcctacctcctccagcttccagtggctcctgacattccttggcttgtggctgcatcaccccaatccctatctctgtcttcccctggtcttttgctcaaaatgtctgtgtttagtttccctgtagacacctctgcatcactctcataagatgcagaggtgcgacatacaggtgttgagagcccacttagataatccaggataagctcctctcaagatctgtaacttggctgggtgcagtgtctcacacctgtaatcccagcactttgggaggccaaggcgggaggttcacttgaggtcaggagttggagaacagcctgggcaacatggggagaccctgtctctactaaaaatagccaggcgtggtggcacacacctgtggtcccagctactcaggaggctgaggtaggaggattgcttgagcctgggagtttgaggctgcagtgggctatgactgcaccactgaattccagcttgggtgacagagtgagactgtctcaaaaaaaaaaaaaacataaaacataacttaaatcacatctcttgccacagaaagtaatactcttttgcctacatataaggtaatatttacaggatccaggggttaggatgtggacatatctttgggaccactgacagccatgaagcaatctcataattttcaaataggttctgtcctttttatctttccagtcttttggaaagcatatgcctatattttcaatccacaattctatttttatttgaggtcatttcatttctggtttttattttttattgagacagggtctcactctgtcacaggctggaatacactagcacaatcatggctcactgcagccaacttctgggctgaagtgatcctccagcctcagcctcctgagtagctggaactacagacacacatcaccatgcctggctgattcattttttaattttttctagagacaggctctatgttgcccaggctggtctcaaactcctggccccatgcaaacctcccgcttcggcctcccaatgtgctgggattataggagtaagccgccttacccagcctccagttttattgtgttttgttttgttttgtttgagacagagtcatgctctgtcaccaggctggagtgcagtggcacgatctcggctcactgcaacctctgccttgcgggttcaagcgattctcctgcctcagcctcccgagtagctcggattataggcatgcaccaccacgcctggctaaatttttgtatttttagtagagaccgggtttcaccatgttggtgaacacaaagtatttgagagctggtttaatatacagccagctttccagaaatgcaactactgtgttcatcaagggaaaactgaacttcgttttccttaaaacttatcaccagctggtcatcattttgacaaattctgtcaacaacagccatgtcattcctggtatctgtattggtcacatctgaacgacacacgccctgcatgcagccctgcaggtactggctgtataacacgcctggcaagaattcccttccaccctgtgtcccggaaacaagaaagtgattgtgatccactcacctcagcctcccaaagtgctgggattacaggcgtgagccactgcactcggcttcctcccgttttttttttttttcaatgcttatattttactctaattaactgagtcaaaaattgagaatagttgaatacactttcatgtaaggcgaatcatttagccgatacttaactctgcatttgggctaccatgccgctgtggttagcggggcaaggtgatgagccctgtctcaacacacacaccccgcctctccccagcccacttacacgcgtccattcccacgcaggtgtgggggccttagaggattccctcttcttcgtaaagtgagaatgggctggactcggcttcactgcccaacaactcctttttttcctttgggaaactgtccttcccattccatataaccttcatgggctgagatcactcaccgagctacaggaagaggcccattactatggccccatcagggttctgcctgggacagtccataaatgctggaagagagaggtgcctttccctactgaggctgctaaaaggagacactgcaaactggggctgctggtggcaatcttacactctcagtgaaagcctgcctggctgcaggggaaaccaatgcacagaccagcaggggcaacatcatttgaacccctggagacagctgtgcctgaagcccacatggctcaggcacatgctcaagccactctgatttgcttgttacagtcaagagagtccttggccgggcacagtggctcatgcctgtaatcccagcactttgggaggccgaggcaggcggatcacctgaggtcaggagttcaagaccagcctgaccaacatggtgaaaccccgtttctactaaaaatacaaaaattagccgggcatggtggcatgtccctgtaatcccagttgttagggaggctgaggtgggagaatcgcttgaacctgggaggtggaggttgcagtgagccaagattgcaccactgcactccaggctagctaacaaagcgaggctctgactcaaaaaaagagagtcctgactgaagctgagaggctggtggacagctgtcagcaagcagtgtttgtgagtctgatgtgagctgcgaagtccacacctgatttcagagctggtggctctttttccaatcaggacagctccaggcctgagttttggtgtggtctgtacctaaccgctgtgtcttaggtcaggatctctagaagccaagcccaagacaggagttctcaaatgccgtgatttactgagggaatgccttcgggggaacctgccagtgaggagagctggaggaggcgggcaggggcgggagctgagccaggtgggggtccctgctggagtctagcctcagcttgaccccacaggggctctggagcaggaacagcacactgcattgtcccttgaggcagtcactggctgcagttgcctctgggagcagagtaaaagtggacaggcatttctgggagtacaattccctagagaaggggacagctctgagctgtgttaccagccaccatttccaggggctgggggatgcgctgcactggcccagagaggtctctgagcaaggcccccacaacctccacttcaacctcctgccaaacctcaagcagattgagaaggagccgctagagacacaggaaccctttcctcatcagaagtctccaagctttggaggaagaaggaggcaggcaggaggcgggaggatcgtttcaccccaggggttcagggctgcagtggactatgatcatgccactgcactactgcctggtgacagagtgagaccctgtctcaaaaacaaacagacaaaaaaaatcaatgctcttagcatctgctgggtccacattgggcagtgtgtgtgacgtggtccagctgcaagggaggctgggaaatgcagtttcattatctgagtactagtggagaaagaagagacgaaagcaatgagaatccctgccatgaggaagtgagagctgattcatttcagcttcctaaggtgaggtgatggggcccagacaggttctgaggccactggaggtcacacaggccagcagggatacacccagtggtagaccctggtctccaggctcccaggacacagaccacataaaagcaaggtcagtctcagcacacagatttaataagcactgtatgttatgcctacagctgcaggaaaccagggggaagatggccacagggccaatgccaggcagggcacagggtggggcccctcaagcagcatatggggaacaggaaagactctgccagcatggtcatggtgcacaggtggctacaatggtgggtaatgcccagttgggaggatgcagtgttagacggggtcttgtccgatggattccttagggggttatatgcttgggaggctgatacaatttcctagaattctgggattctggatggtctctgacctgctctaggggatctgggtctccagagaggtctcagctacaaaagtgaccctctcctgaccccaccttgtgggagaccccagtggtctcaaggtctgtgaataaagggacttacaaccaagatgcctcctgacagggagacgggggctttacagtcaggccccagcctacatcccttgccccaggggacggcacacagggtgtccaaacctacaaagggcagcggagtgaaagcaccagggccggcttggggggtaagccagccccttctcaaggggccagtaggccacagaggcggcccaggcaggcagggtggagtagctggctgtgctatccgaggtcgctgtcctaatcagagcagggccgggagagccaggacaggaagtatggagagcagggagcgtctctccagggccctgcctgtggaggacaccttgggggtgggagccaagtgcagacttgaggcaaaagcctgtctctgccctctgcctgcacccccaccccaggcccttcatgcctccacccaggcccaccagggacggaagggtgtgaaggtgacagtgggggaggtgccagctcaccctcccctccctgcacctacctgcaggaggctgtgctccgggtctccctgggggttcagctggtccagcaggactgggggccacccgctggcaaaggcctgaatggcaccatctatggagggaggggtccagggtggacttagctgggaaaccctagcacagggctcagcacaggggacaaagtgagagaccccctgctgcaaagactggctccttggacagaaggtgtgtggtgaggaataacaaaataataagcacagctgtgacagagtgactcaactgtacagtcttccattctcccctctaaaatggctcccagccaggcggcttgggagggggcagtaagcgccgcccccactccccctccactcccccccgggcccctcacccaagcagcggttcctggtaaagagcccccggaaggcttcgcagtcctcacgccggttcccgctggctccgcagtcgcaccagggcgccacgcgcgcgctcacgttgtccacgtagttaggggtgacggcggtgcctgcggggaccctgagggcgaagtcatcggcccacccgggcggagatatcccctggagaacccccgccctcgcccggatcccggccgcgcgtacccacgaggcccgcgtaggcgcgcaggcagcgggcgccctggtccagcaggcagccgtcgggggcgctgggcgctggggtgcacgagacctgaaaggccaggaggcgaggcctgcgcggcgggagggcggtgagccggagagaggccccgtccccaccctcgccacggccccgccgccgcccgcgcgcacctgcagacccggctgcgctcgcagaagtttaagggctcaaggcaggagggcggcgcggggccgggccccgaaaaggcgcaggagggcacgaaggtctggcgccgacgctcggcgcacgcggggcccgcgcacgggcagaagagcagtgcgtgggtgagcgcgggcggcccgcgggcgaagaagcggcgcagggcccggcggcagcgggcgcggggacagcccccctgcgcagcccggcccaggcactgcgccacatactcggagcgcaaacgctggcaccgcgcgtccgccgtgcaggcttcggccgcgtccacacatcggttccctccgaccgagctcgccgaccctgggaaaggcgcggggaggctgcaggtctcagtgcgccccgcatgcacacttcacccagcccatccacgcccctggaaggtgggggacactgaaaacccgagaaagcatagagtgtggcccaaaggcggggccgactggcactgatcggctctccgtcgtggggatgggcagcggcgggctctggagggctctgcaacacgtcagggatggagagggagcatggaggctgagccaggccttccccaaaagctgaagcccctccttcccaagcatcgccaccattcccagaacggccacacacgtccaaagacgggtccaggggctcctcctacactcaatggcactacctgacgggggaacaatgaggctttctggggatctggccaatggagactttaaaaccagagatggcatagaagacagtggctgctcctagtctcttaatgacaggctgtgacgccatacctttcctcctctaccccctccacctcgccgcttatcctccaccatggagaccctttgggtgggtctattgatttctgtttctccctccttcctgacctcctgctcctcctcctgccccctgcatgggtgtctcccctctccagcccttcctgatgctcccttgagtcgaggtcatcctgccccatctgcactggcttcctccagcccctccacccttcatcagccaggggagagcggatttatgcaagagctctggccactgaagaacccactgagtcctggctgccagggtgtggattcagagaagtccctgacgacacaccgccgggctttgtcctgagccacaggacccaagggttcatcagtggttttagtgaccaggccccatgataggtgacaccctgctggagacagagaaggtgtctggcaccccagtcacacaccaggggttgggtagacagagggtgagctctgcccacatgcccacaggctgtgtctaaatagagccaggataactggaagttgggacttggcagccccttcccaggatttgagtcacaaaaaccaggctaatcgaatcccccccagcctccacaagttctccagtctggagcatctcattcagactagaagcctttctggtgcccagcctgagtggacgtgcacagcctggcccctcatccatctccctgtgtgtttgtgcacaggggctacggggtggtatccccccccagggctgggagcagggtgcctctctggcaatggctgtgtctttcaggctgtggattggaccgcgtggtgagagtgattcctgctgtgacttcagcacctgcctctgcaggaaggatgaacatagctcgagggcttagaatggtccctgagtggaggggatagatggcagcggggtgagtcctcccttcctcccctccccaaagggcacactttgctttcctctcacagccttgccaggcacatcctcaggtctcagctgcacacaccagcacctgatgttgcctaagggtttgaacccacctctgcctgttgcttacctgtcacccttgcccagaacacccttacccagtctccaccctcacagcagctcatctcagctgcaagatctcctccaccttaaacctttaccagattccactttccacactacactcggtaaagtgacccctccctccgcgacatccatgcagggacggagcagtccaagaaggtggaacaacagatgaacaacaccgaagattcaccaaggaatctgtgtgatgggtaggagggctggggctgaggggagggtgagagagttgggagatggcagacaataggcaaaagttcctggggcacgggtgccaaggctggactcgggagggggatggcggtggtggcttgacggggctgggaggtgaggtgctcagcacatagtggggtcaattgccaggggtccccaagccgtgcgcacaggcctctcactggcaacccttcctgtggttcagcttttcctggaatatcttgccctcccaccttgtcactgaggagcagggacagagaggccagggttcggggtccaggggaagagaggggcgctcacccagtaacagcagcagcagcagcgcaggccccaggcagcggaccatgctggaccttcaacagagaaggatggctggcagagtcctagtctgataggcgcccccttcctagtgcctctggagcaggagcacagtgaaaacccagctgtctgcgctgatacccctgggaggagcctttgctgcagcaactccccgccctcattaagtctccaccccaggctgcaccgtcaggtaaacctgggagccacgggattcggcgcctgaatgtgcctagacggaccccaacacactagccctgccacgcacacccagcaacacacagacaaccacccagggagacctggtctctcccatgctaaactaggaacgctatgaaacagcatttttcttttccttttttatataattctattttaaacttaaaaaaatttttttgaatagagttggggtctcgctctattgcgcaggctggtcttgaactcctgggctcaaaccatcctcccacctcggcctctcaaagtgctaggatgacaggtgtgagccaccatgcctggcctattttaaacttcttattgttgactaattttagacttgcagaaaagttggaaaatgtaagcattttccttacacccttcacccagcttcccctgatggtccatcttacttaatcatagtccaatcattacagctaggaaactaaccttggtacaatcctgttaacgaaactgtagactgttttgcatttctcatttttccactaatgtcctttttctgttccaagatccattctaggatcccacatcacagttgtctccttattctcctcaatctgggagttccttcatctttccttatctttacccttgacacttttgaagaatcctggccagttattttgcagaatgtttctcttgagttgtctgatgttttattctgatcagaatgagacacagcattgttttgactaaccaaaaagttattctataagtaaatattgaggttaaaaatctcatccaacctgggcaacagagtaaggccctgtctcaaataaagtctcaacactaagatttaaaaagtgaccagaaaagcccccactatgatttgtcttgacttttttttaaaaaacaaacaaacaaacaaacaaacaaacaaaaacgatgtcttgctctctctctccctcaggctggagtgcagtggcacgatcttggctcactgcaacctccgcctcccaggttcaagcgattcttctgcctcagcctcccaggtagctgggattacagacgcccgccaccatgcccggctaatttttgtatttttagtagagacaaggtttcaccatgttggccaggctggtctcgaacttctaacctcaagtgacccgcccacctctgcctcccaaagtgctggaattacagacgtgagcaactgcgcttggcctcattagcatcttaaatctccacacaggggtgtgttccttactgttataaggagcaaaggatcagtttgaggacaggtaaaataaaaatgcgcttgctgcctagagggagaagtccctgctgaagatagctttgcttgaatgagctcaattgcaatgccagtgctgaggcttgttgactgtacggtcaccacagttgctgctgcgcgcctagaacatggtcactttcttgactacctatcctgtctcagtacatctgtctgtggtttgtggtggtccatttcctaatttttttaatgaatcagaagactgtgatgtgctttccgctgtgctaaccatggccgctgaagcaaaatgtaaaccaagatgcccctgcagtggttgtgcttcactctacgacatctgttaccggaaaggggtccagattcagaccccaggagagggttcttggatctcgtgcaagaaagaatttgagacgagtccataaagtgaaagcacatttattaagaaagtaaaagaataaaagaatggctactccatagagagcgcagccctgagggctgctggttgcccatttttatggttgtttctggatgatctgctaaacaggggtggattgttcatgtctcccctttttagaccatatatggtaacttcctgatactgccatggcatctgtaacctgtcatggtgctggtgggagtgtagcagtggggaccgaccagaggtcactctcatcaccatcttggtttgggtgggttttagccagcttctatattgcaagctgatttttttggttggtttggtttttgagacggagtctcgctccaggctggagtgcagtgacatgatctcagctcactgcaacctccacctcctgggttcaagcgattctcctgcctcagcctcccaagtagctgggattacaggcacacaccaccatgcctggctagtttttgtatttttagtagagatgaggtttcaccttgttggccaggctggtctcgaacttctgacctcaggtgatccgcccacctcagcctcccaaagtgctgggattacaggcgtgagctaccgcgcctggcctactgcaaactattttatcagcaaggtctttatgacctgtatctcctatctcatcctgtgacgcagaatgctgtaactgtctggaaacgcagcccagtaggtctcagccttattttgctcagcccctattcaggatggagttgctctggttcacaggcctctgacacatcctcttgtgttttggcgtgtgggaggaaagaggggtgagggaaggaaactcaaaaccaagctctgaccacacagggcaggtacactctcccacctgtctgtgggtgccacaagtcaagggaggggcagagagagaagaaggtgtgacagatggccgcaggccacagaatgtcagaggaagcccagttcctcccggggcagcccaagtagctggtagttgggtggccaaacagagggcgtcacagctgagctgggctcgctcgctacccccagctcagcgtccactctgcccctcagtacctcctgctcagcctcagggtccatgcctaccctcctgcttcccagtcacttctgcgtgcctcctgcttttctgctgttggccccatgccagctcctttctgctgagcttctcttctccagttctgcagcacagccaggtgatcctgggctccagacaggcctctcccccagtctggggcctcccctcttagagccctctttccttcccacgtggcctccccagggttcgccactgaatggagaaggggtggagggggtgctgggcagtcttggggattagccaagagggcagagttggcctccccagggtcccttgtagctggagtcccgcggggtctagacaccccctcctgaagggtaagagcgggggaggtatattaacgtgtatttttagagtctctcttttttttttttttttttttgagacggagtcttgctcttgttgcccaggctggagtgcagtggcacgatctcagctcactgcaacctccacctcccatgttcaaacaattctcctgccgcagcctcccgagtagctgggattataggcacgtgccaccacaccgggctaatttttgtatttttagtagagatggggtttcaccacattggccaggctggtctcgaacttctgacctcaaatgatcctcccgccttggcctcccaaagtgctgggattacaggcgtgagccaccatgcctggccttagattctctattttatgatggtttaacatctcggggtgggggcttgttggctggagagaaactgcttgattcctggagatcagaaacaactcatgcctttcatatgcaaaccgaccagtcttgagtccatacaccaaccacccccttcaaggaactctcacatacgaaaccagtatttcccctgccctaaaccagctcagggccaggcacctacccagacaattagagcccaaccccacgccccaaaccccaccagaatgattcaaaatgccaatcctaccctcttcccctggcctgccttgcctccccagtggaaacggcactgtgggctgtggcctgtgccttccactcgctcctgattctgtccctggaccaaacctagtgcctccccactgtggccctgcatggtgggaaactgtgagtaataacttatttcaacagcattggcctctgtgtcatcagtcaccttcataaattaaaattttgcaactacaactgaggcaggagggggctttagagagcactctcacctggccccttgggcttgtggagtggagcccgaggtgaagttgcctccctgcactcagttttgggatggttttgttatgcggcaacagctggctgatataggccactcagcagctcttgcatgaagcaatgggagaatgtgaacgcccaagggaggcaggagtgacagagcaaagagggtgttcaaactaggcaacaccgtcctgtgcccagacacatgcctggggctctggctgccatctaggctcggcctgccagccaccaccccgtcagccaccaccccaccaaccaccaccccgccaaccaccaccccgtgtggtcctcagggcaccccatgggttgaatttacatacagaaaagactaaccaaggtccaagtgtaatatgtctttttaaaatttattttagagacagggtcttgctctgtcacccaggctggagtgcagtggtgccaccatagctcactgcagtgttgaactcaggctcaagtgatcctcctgcttcagcctcctgagcagctaggcctacaggtacacaccaccacgcccagctaattttaaaatttttctgtagatgcggtgtcttgctgtgttgcccaggctggtgtggacctcctggcctctggtgatcctcctaccccagcttcccaaagtgctgggattataggcatgagccaccacgcctatagccaacatgtctttcttttgacttctactttggtatcttttcttaaatggttccctctgtccccccgacacacacagaatgggggagaggctgtcagattctgagctccagaacctcaggtgtagcactgggattgggggtgggggctcaggaaccacctaggggagaagacagggtgggaagaaacaggaaggaaggtccccaaaattatgtttgtttgcagaggccagccaggctccaggggagtgtggactcagtcgaaccatagggccccaggaccactagcttctggccagcagtcatgccctccacagagctgggtccgtggaaattgcatgtaggagacacaccagactcccaggacagagcccttttgggatggccagcactacccagcctccactggtgagggaggtcaggggctgtgtgacctttgcttctgggactgatggtttattgagctggagagtgtgcccagcagtgttctccagccctcaggaacttctagtgtggctctgggttcctggagtgggtgggtcgaagctccactcggggaagaaacttccaagctgcctgcaggtgctggaggtccggtgattcactggctctgcccctgcagttcaagttcctggagtggctgtcagtggccacctgtctttaaatctgttcattttaggagctacctctcaccagaggcaggatcttggcatctggacttgatctgctgagaatgaggaggatatgttgtcccctaaggactggggccccaggctgcaagctgtgtggcagagagcccatcctcactcagtgaggaccagtgatccaggaaaagccacagcttctccctccccagcccaggggcttccagcatcctggtctccatgataaccaagaggtcataaactcatttccataataacctgagcccagaaacctgattagggggcagcaaactgaggggtgggagaggtgggagggtgggcgatgagagggggaggctttgaatccaggtccctgcctaccttgggggtcaggcgagactgctggcagaggcttctcagggtggctgctgggctcatgagagttctcagggtctgggagaaatggtggagggtaaatgttgtgaatatggtcagcaggagaccctggggctggggaggggcataggggactcaaggtgactgggtgctgcccatctggaaggaggcaggaggcatgagcccttcccttctcccttccctctccacctccccctggtgcctcactcacccaggggccagggctgtccagtggctgtggggcccaactccatggggtgaacgccgcccagggggtggtccctgtgtgggccatctttggggctgagcaacgtgataagagtccaggaggttggcacagtgatcctgagtgggttattgcctccccgcagcatggtgtccagcccagggagttctgcgtttactgagtttcttggggcacccatctgctccaagtcaccctctcagctcccttcctgctccctcttcaggggagccttgggatccaggctccaagtgagcctcatgccctcggctggcacctcctctctctagtcctaacatttcctccaggctctgacaccacccagcagcctggcactctccagatgctggcatcgctcagcttccaaagaaccttggatgtccgccccttcggcagctatgtctgctctccttgcccctgggtgccctgctgcccttgatgattccaagccatctttgactgtccccatcccatcccccaaggccttgtcatttcctgtgatgttccttcaaaacattctcccctgccctgagactcccgcctggggatgagaagcagccgccaccctctgcagcgccccctccgtgctgacacgccaggctctggccaccttgctcctctgcccacagaccctcaatcacaactcgctttgtcagggtcctcttagctgccacccggggcccaggtggtgccctgcccctgtctgttatgccctctgcccccatctctggcccaaatcatgccatctcccttggcttgcccggagcactcccaagaccaggctatgtcagacatggccacagagtgcctgccctgcctagggccctggtgcagggtgagtcctaggacagccatgcttagtattatgtgactccccactccgccaccacccaggtcacagagaactgggttaaggcagggccctggcacaggggcagccagcaccgcagctgaccagtggtatggagtgaaaagatgtgctgggcccagcatttgggaacttcaagggggtgacagaggtgatttgtgcagaggaagtggcaaagggccgaaaactggtgagacagaggctggacaggcctccgggggcagcatggtacagggactgcaatctgagccagggaaaaacagggcgaagtcaagggtgaggcagccagctggtgggagaagcaggagagtggacaagaggagctgtactgggaggtagagggccatgccttgcggtgctggtggtggggcagggatccaccccctcgcttgactgggaggccactggaacctcctgttcaaagctacttctttccatggcctctggggctgctctctgcacctgggggcaaggctgagggcctgccccagctcccacagccccagcagagctctgaggaggggaaccgcaggagtaggctcaggaagcaggcgctcggagcctacccactgcacgcagggtcccttctgcagccccagctgcatcgctgcagatgggctcctgggagtcggtagcaacaccaggccaggccggcccctgggagcagaggcagcaggacgctgaggagcatggccagcaggaaggtgtcatggtctgcggggattgggggaaggggcgctgagtcctgagcaggtgcaccaccccagctcctgcccacatgccccccactggcatactttcagcctgcacagggccactgtccatgctgccaccaaagcctggcttgtcacagaagggtggagcccagcctggagcacagtggcagttatggttgctattgcaaacctgcagagaagagaagaggagggtcacgtaggattaggaaccccaaggtcacccccactcctcgggctctcaccccgtggctgtggcaggcagtcaggcagcgctgaagctcctggaaggcattcttcctgcagcgcctgctctggcacacctacgggcagtgcaccaggcagtgagggggacactggcctgcgggattcaaacggcaaggaggggtcgggtgggcagagctcaccattctaggtccacactgggtgcctggctctaccaggcccaggccaagcaggtccagctgggcactggggagtgccaaggctccccgacaagtcacttcctggccatctaggtgaacggtagagtccactggcaccatgtgcggtgcgagcaggctgggctttccaccctggcactgcagcttcccacacagggcatccctggggaggaagtagaggggggtcaacagctgcagtaccccccttccccaaacccactccatagcttctgctccctccactcagctccactccctacctccctgcacagggcaggaagtggccctcgctgtcctggccgcagtttccatgagcatctcccgcagagttcaccacctggaaacaggcctcgggagctgggtgggagcctgaggaagcatgggccaggctgggggcagctcggagaggggctgcgctcagcgggcctcagtttcccctgcccatcttccccacagtagaaaactggccccaccagaggatggggggcagggtgcaagggtgctcgtgtcctctcaccaggcccccagagctgctggcactgctgctccagcgtgggacatgcgccatcccagcagtagccactgcccctggcacagggtgagccgtccagtaggtaaacgtctgggggacagtgggaggaggtgcccgtgcaaaactcagggaggtcacagtcacccatggcctggcggcacagcgctccagccggcttcagctgcgcaggtgacgggtggtggggaaggcagagagaggccacgtgcagtgagaggtccatgccgagagcgcggctcggagctgggggagccaggcctacccaagcccagcacccaacgggggaacctgagggcaccaattaactaaggccaacaggccggctcccaagctccccgaaaccctcaccctgaaccttccatgccctcaccaggcagcgcacgcagcagtccccgtgggcgcactgggcccccgggcgcagcgagcagttgtgagcaaagcagcagaggtcgcggcactcctgggaccagaaaggcaagaagggcccaggtgagggcgcagcgccccagacctgagcggagagggcaagtgggggccgggcgagccgacttaacctggccagggccgcagtcacactcctcgcccgcttccacgaagccgttcccgcagagcgccggcggcaccgggagtccggggtccggggcattggagaggcaagcgccgccccccttgcggaagaaggcgcgcagctggcggcggctgcaggcgctgaacacgcgcggaaacgggtgcctaccggcacggggagggcattgggcatggagggacagtcccccaacccccgcgcttctctgatccccacccctgggcttggctacagccgccagacgcgcagagcccagagaggggaagtaacccgcgcaaagtcacacaacaagcgggacaggggacgatgcggccccaatagtgagcagcccgggacccaaggtggaatcgcgacccgacggtgctcctcccggtgtaggagtaacctcgccaggttactcggaaaataatcttcataccgttgagaatccactttgcctgagcttcttccctttaagcctcataaaccaccctgaagcggacactatgatcattatccccattttacagaagaggaaactgagggacgaccaaagaaacgcagcggaggaagtccccaggactagccgccccgccgcagccccgaccccccacccgcgtacccggtggccgcagccatgacgcagcctccggactcggccgcagcctccacgcagcagccgtcggggtcgtggctgaggccgaggctgtggccgatctcatgggccatggtggctgcggcgccgatggggagctccgagtggtcctggggggccgtgggagggcggtcactgcggccgtagagcctcctgtctctccctcgcccccgcccgcggggctcaccgtgctcacgcctcccgagctctcggcgcggcacatgccctcgacgggcgccaggcccactgtggcgccctggaaggcgcggcccctgggggcggagcgcggcgtgaccaggcggggccgggaggtgaggccgccccacccgggacccgcgtccgggtcagaggcacccacgtgagcagctgcgcggagtcgtggggccgctgcgcccacagcccccggcgccactgcaggaaggcccagagcgtggcgttggcgtcctgcgtgacgcggctgcggtcccgctcggtccacacctccaggccggtcagcgccacctgaatgtccagagtcctgagaagctgagggcgaggcggggctgaagccgggacagggcgccccatcgcgccggtggtccttcgtggggcgcccttcctcttccccaaaccccaccagcacctgcctgtcctgccgccgccacccccatcaccgctctctccccgccgcccccaacctggtccacgtagttggcgacttccaggagacgctgtttggtgtggttcaagtttcggtgccgagtcaagaactgggaaggcagaaatcccggtggcttgaggggctgagctggccccatccctgaccccgccaacccctggggtctctcctcaccagggtgtggtctgccacaatgtacagttccaggtacttccgggtcctgcgcgcttctcgcctgccctgcggaggtgcaaatggggaccctgagtggaagctgctgggcttgagccctgacccccaaccccagctcccagaaggaagtttaacatgttttctggaacttgtttcttcagacttcaataaaaatactgggactcgaggcctgtgaattcctgtctcttctgatttggagggctatagatacagcattcccactcccatccgatcgatgcccctgaccctgctctggggaccaccaggaaggctggtcatgcccgctttgttcccaggatccctgtggccacaggttcctttccaggtgagcagctgctccatccgaaagatctcgtgggttgagaagtccttggagccccggggtggccagggacgcagataatagctggcattcctgctgagggtgatcaggccactagggtgcagaggggtaggagcgggtgtgagggagctctttccccatcccaggcccagcctcctctcccagagctcacctcatcccagagcaggtgcagaggactacccaggagtcggggaagccccttactcgcccttggtagtggcaatgatcctagggaggaaggggccagccccaaatctcagccagggctggagcaagaggggcaagagggagggtgtggtaggggctggctccaaccgccccttaggaatgcaaggaggagtaggggtaggaatggtgggggggtacctctggcggtgcatcccagagcccatggaagcatctcaccgtgtggttgggggccagcaccactggctgcccatctgggccgtagtgggtttctatgtatcctggggccagcagcctgctgagagggggtgttacagggaacactgaattcagcttcctcctgcctcctccaggatgtctcccagccttcctccctaaatgctaatggagcagctttatgagtgagacactcacagtgtgtcttagggaagggacaggagcaatggtgacttgctcagatcagaaactcttggggctagaggaaggagccttggtgatggcttagttgtgggagatgtgaatatgggaagcaccagggaggacgccggggaggagtgggaataggggaagagtttgtggttcccaggggacctgcagcaggcagcaggatccacaggatcgggaggggaggagtcaggagacactgccgaagaatgggacttggagttggggaaatgcggtgacctcccccagttcccctgcctgctgccctcctttgttgggcatctggtcgaccctcttgcccccacctgccctagatccttgaaatattttcctcagacttctagaccccacatacctcccacctgtccttcagtgattgatgctcaccccctgcctccagagaaaacagaatcgccacctgcccacgctgcttccaccctccctgccttctccacacccactccggtaatgattccatcttcaggctccatctcaacaggatctttcccacacggatggatcaatcataagtcaatctgtcttctttaaagaaaatccttaacccaacctcaccttggcctcattacctccagaccacccgctaatgatggctgcttcccccctcccaggcattccaccacctgccccagctctgccccctacccctgccccacacacacccgccaccctaggaggtaggtgatgtgaccaccccattgaaagggtagggacgtcgggaaaatatggttgggcacagtggaactagagtttgttccctgtccatccgactccacgagggagaataaaatacgtgtcaagtgctcagaacagcgcctgcggtcaagcactcagtaggtgatatatactgataacataatctgggtggttttaagagcctgcgctccagcccggacacccaccccacccagcccaaggcaccttcctgagcacaggtctgcctgtcccttccccagctcaaaatctttggctctggatggtccaggacactgagcaccaaaatggccttctgtgatctggccctcctgggactctccaaattcattcccccctgctcccctccctgtagatggagaccttccaggcaggtcagacaaactgctgcccccaagtgtagccactgcatctttttcttttttcttttctttctttttttttttctttttcttttttttctctttttttgagacggagtctcactctttgcccaggccggagtggtgcagtggtgtgatcttggctcaccacaacctctacctccggggttcaagcgattctcccgtctcagccccctaagtagctgggattacaggcgccgccaccacgcctggctaattttttgtatttttagtagagacggggtttcaccatgttggcaaggctggtctcaaactcctgacctcaggtgagccgcccgcctcggcctcccaaagccaccgcatcttggtccctgccattcccttagcctggggtgccggctcatcttttccctctaggatttctttagactcagcatatcttgcaaatgtccactaggtggtgctcactcatcgccagcagggagctaacaagccgctcctggggttgggagggcggaggtgccccacagcggggctgacagcctcagcggtcctcttcagcctccagggagccaaccacaggcctgcgtgactctccctgtcatctgcaccctctctggggtcctctgcccatccagccacccgcacagatctgtgtcagtccctgccccccaacactgatcccctcctcccagccctaccccagcctggcactcactggttcttctccagctcaagcaggagctcctggccttcagcctccagggccaccagccccatgtctggcttcgagacctgggcaagaaagagtgtggagctgagatggtggcctccaggcctcctgcctgccagggagtaggtggcctgtggagccggctggggaggaagttcttggggagaacgtgggctggggagtcagcaggaccccccacatactatggagggcgtggaggaggtgagaacatacaaagatgttcccaaactcaggatgtttgcagtcctgacaacagccacttggaagggcgttggcacagcctgccaggcacaccagcatcctccctagagaccagaggtcccagaaaggtgcccctcccctggccccgccctcttctttcatgcccagaaggggcatcaaaagcaggggaagacagaggggtgctgaggacattatgggggcatcgggtagccatggtcagggcctcctcagagcctctgctacctgaggcttggttccaaatgagctgctgctcatttcctatagaattcaaatttgactcctccacttccaattttggcaaactgctccctcttccaaagttttcctgggcctccagcagcccccgtccctccggctccgacacctgcttcactggacccacgaagtaaacatggacgccattccagccaagagagcacactggctctcagctaggtgtcaggaggctggcttggacggccagccctctctccttcccccaccctcttggcgtctcccaccctgtgggaacaccccacttcccccttgtccactcagcctggctgggggcccagagttggagccggcccaggagcttcctgggaggctgctgcgccttcggaatgtttaacccccgactccttttctccaaaaatgcactggcctggggccctgtccaagggtctcagagtctttggagggagttcttccttcgcaagtggggagcagatggtccttgcctccctggccacaggccccacaaggcctccagcatgagctcatgaggctggaatgccacttgctttattggggaaaggtctgcaccgggaaaaaggccatactcgaggtccctgttcctctgcagcccctgctatctttactcttgccctcctggtaccctgccccttgatatatacccctcatcttgaaatgtgagtgtttcctgccttttggaggggatacctagcctctactctttcttctgtaccatcttggcaggcttcctgggggcaggggcccaccggtgggggaagcagagcccctttggggctctcctcttggtcacagcccaggccagacagacagggaggcccagaggcagagtgaccccagtgtgtgtccagccttcccctcctggggatggggagggcaatctcaaagctcaggccagtgccgtgcttgaccagtggaatgggggccttatgggcctaggggatcccagtgagggccctgggttgggagctgctgggtctctgggggcctctcagccttcatggcaatgctcccctgccttccctcttgctggatttggacagtagggctgaaaattccaaacaaagagggctctctaggaggggcaggggtgtagccaatggtttaaaatcgttcagaccttagtgggtctcaggctcccagcctaaagagctgtgtgaccatggacaatttccccaagctctctgggcttccgtttgcccctctgtaaaatgagcatatcaaggctactgccctcttagtttgcagcacagatattatggcacaaacagatggggcatggttattctggaagcgtgtgaagagcgggattgggaagaggctggggcagagcgtcctgcagaagaagcacatggggtggtcttacatctgggggacatcaggagagtgaccactgccccccccataccagaagtggattccacaggagccagtgaggctgaaggttcaggccttcgtggcagggccctgagagggacagcagtgtgtccacagggtcacatgttctggtcaactttgcaaaaggttttctttttggtgcttttttttttttttttttttttagaggctcctgaaaagcttcaggacccacaaactctggacccatttctgcctggtgggggtgggggtggcccagatcatccagggagggagggaaagagggaggtggggtggagaaagctgaaatgacttccatgtgtgcgggctcacgagatccagatgtccaaaccccagtgccttcttctgcccacttgaggggcaggggaggcaggggcctataggagtagtgacttggtggttctggggaccccagcaaaactagaagctgtaatgtagggagagacaaaagggctgggaggttcagggcccctgtggagggcggggagacatggcactgaccggctcctccaggctgacggtgcgccagggttgtccatccaggacccagtgcggggtgactggctgcccagggatatgtcctggagtaaagacagagcacagggtgagggggacctgaggaacacaggggcatgggacaaagcagagggaggggggtagaggacatccccagggaggcactggaggccttttggggcagacttcaccttcaacacgcgtgggctcagcctggagaaggagggacgcccgtgggcatccttggatctgaggagctatcaaggaggaggaaaagagaaggctggaaagggacagctcagctggggacacgggagtcccctgacctttgtcggggggcaggcttgggctcggcgatcacaaggaagaggccaaggccgccagtgcagaggggaaggaaagagcgcggcagccttagggatttttagatgggcagcagatgcctttagggtgagagatgtacgaagagaggacacttgtgccccccccatcatctgagaaaaacaacagccagatgttgccttgcgaggtccaccttgcccagagctccctcggggactctgtcctggtggcagggttttggtaccctggcccagaaggcccctcctcatctcttcaaggggaggggacgcttccggacggagccttggtgctccctggccgggtgtgcctaagggggctctaggaggaatcccagagccaagcattactcagagggcgcctggaatgttcccctggaatgctcccagcccctccactggccccaaccactctcacaggcccgccctgcaggagccaggccccaggcacccagagcctgcagcagccctccttcccccggtacccagtcccagctcccagaacagacagcctcccccctccacgcagccctggcctcagtcctgctgggctgatggctgcctgtggaagtgactcagctcctgctaggccaccccaactccttttttctcctccaccttctctcccagactacaaacatcaaagacccttcctccaagaagccctccttgattggatgagtgaattgccatcaggcagatgagggccgagaggagtctgccaccttggaaaggaggctagaggggccagtgcagggagggctctgagtggatgtgggggaggggaaggaggggaggtctctcagcccagagagcacttaactgagagtagagaaccaagctttgctgctcctaggcctctaagggtttggggaagaggtagggtgggcccgggcacaggtgtggtgtgggtgcagtgtggtgtgtgggtgctgtccacatggccttgcgcgcacgtgctggccacgggcaccctgaccccaatgagggagagaggggcagagctggagctggagctggagctccggtgaccgggtgaatgggggtggaacccgagggagccaggctggtattgggcacatagacgcccctctcccaggggtcccatcacctcccctgaccccaggatagggctcagaggggagggagcagtggaccgcctggggccctcccctggggccagaacagaccaggcccctgtacctgtttggtccccacacagtgctgtggaagccaccgcccagtctgcatagcacagcccagccccgcatgccccctccctggttgccctccctgttcccggccaggcacttgctgtgcaggactggctaatcctcccacccgcttgcagaggttgttccagccccatcttaacatctttgtttggaggggttaccccgaggagacagctgcagtctttccagagcactgctaaacagacaccttctatctggagaggcccttctctatctcaccaaacaaggcaacaatataaacaacatacacactgccctgctgccctgggaggagggacgaggggtgagcagggtggaggccacagctagttctgcagcctgagagcaaagcagggactctgggggactcttgggcatgggggcttcctagaggatggagccccgctgagtcctaaggggtggaggagcaggagcgggtcacacggtggcctgcggatggaagctggttgtgagagcgagaatccaggcagagggggctacggctatgggctgggggctgggggctgggctgtccccgagggggagggagccatgccctctgctttgccagcggagtggcagccgggcagtgtgggcaagtccgggcccggggccagcccaagcacacttgagcgtccctgggcaggtcccacggagacccccccaaagagtccccacgccctgacctactggccgtatggtgccggggccgtgagaccctccgcgcgctgacccgagctctgagcagaacccatccccgccaccaccaccgcgcctagcctgcccctcagggcgcaccccgcccgcgtcctcaccttgaagcaccccggcgcctggcactggccagagcagcagcagtagtagcagcagcagcaacggggtcccccgagctctccggggcctccagcccatagctgtgagctcctcggcctctaggcagcggctcgcaactccggctccgcccaggctggattgcggccgacccgtgcccggtgcagcctcaggccgccgccttcggaccttcccgcccccacctcccaccgcccgccctcgctcccgcctcccctccccgccaaccccgctcggagcctggccaggggccccgacggcgcgcgccatgggggagccgggtcgccactcccggaccgccgcccctcgagggggtggagctgggcggaggagggaatccgtgcggcccctcggatgaccggcccgagccgtccctccccgtcggtctcagagggcctctactcctgagaggaggagagaaccgctgggaaggttcttggaggaccgcggcgtggtgggatgaggcggtgggcaaaggccgcctctcgctgctgaagttggccccaggagcgcgatcttccgtggtctcctggggccgatctctgtcccctccttgctacccgtcctgccccgagggtgccctggcggaggttgagtcgggtcatccacctgcactgggtgcccccaaggataggaaggttcaggcaaccggctgccgctgtcttgggggcttcattgctgggcaaaggcgatgcagcagacggagacaacctttcttccctggcggtggccagagggcagaattgcataaaagctgcagactcccaggcctgggagaccctttcggcctcagtaacatctgtttcatgttttaaacttttgttttcctactcggtgcaaatttggatgagatgttaactttttttttttttttttttttgagatggagtctccctctgtcgccaggctggagtgcagcggcgcgatcttggctcactgcaacctccgactccctggttcaagcgattctcctgcctcagcctcccgagtagctgggactacaggcgcgcgctaccacccccagctaacttttgtatttttagcagagacgaggtttcaccattttggccaggatggtctcaatctcctgatctcgtgatccacccgcctcggcctctcaaagcgctgggattacaggcatgagccaccgcgcccggccggagatgttaacttttaagcaaatctttttttttttttttttttttgagacagagtttctctcttgttacccagactggagtgcaatggcatgatctgggctcactgcaacctctgcctcccagattcaagtgattcttctgcctcagcctcccgagtagctggcattacaggcattcgccaccacgcctggctaattttgtatttttagtagagatggggtttctccatgttggtcaggctggtctcgaactcccgacctcaggtgatctgcccgcctcggcctcccaaagcgctggaattacaggcgtgagacaccgcacccagcctacttttaagtaaatctatttgtttttgagaatttggaatgtagtaatttggttagtgaaagttcgagcagtgagagaaacctacattcacatatctcaaaatcaaaaagtacagaaagcatagggaaaagtctccgtgctcttagccctcctcaccaacaggaaaccaatatgattagtttctttcataggcttttagattattttttcacactcaagacaatacagacatatttttttctcttattaacgtttttctgcactttgattttctttttttttttggtcgcttaatacaccttagatatcagtgcgtttagagggtccttgttgttcttatgattattatttagagacagggtctcactctgtcacccacgctagaggacagtggcctgatcatgcctcattgcagccttgaaatcctgggctcaaggtatcctcccacctcagcctcctgagtagctggaactacaggcacacggcaccaggcccagctaaaatttttaatttttctgtagacagggggtctcactttgtttcccaggctggtctcaaactcctggtcttggccaggcgcagtgtctcatgcctgtaatcccagcactttgggaggccgaggcgggcagatcactggaggtcaggagttcaagaccagtctggccaacatggtgaaaccccatctctactaaaaatacaaaaattagccgggcatggtggtgagcgcctgtagttccagctacttgggaggctgaggcaggaaaatcgcttgaactcagaaggtggaggttgcagcgagccgagatcatgccattgcactccagcctgggcaacaagagcgaaactccgtctcaaaaaataaaaataaaaataaaaagaactcctgatcttaagtgatcctcctgcctcagcttctcaaatcgctggaattacaggagtgagtcaccacagctgtccagctacgagattattacttattattactactttggattttcaaatcaacttcattaaggtataatttacacacaataaaatgcacttattttaagtggccagtaagatgagtttcgataagtgtatataactacataagcatcactataatgcagacacattccctcactcacagaaagagccctgtgcccttccagccaaacttccccactcccaaccccagacagccactgatctgttgttctctgtctatagataagttttgcctgttctagaatttcatataaatggaatcatgcggcatgcactcttctgtgtctggcttccttccctctttccgatgtttttgagattcatttacactattttgcatatcaatagtttgttccttcgtattgctgaatagtgttcggtggtttgagggaaccacagtttctctactcaccagtgcaccatagggttattttccagttaggggctcttataattggaactatatttgcacagagagagagagaggaagaaagagggagagagatatttattatagcaattggctcacgtgattatggaggccaaaaagttcccgaatctgccatctgcaagctggagaacgaggaaagccagtggtgtgattcagtttgagttcaaaggcctgagaaccaggagcaccagtatggaggtggctcgagctcagaacaagttggggacaggaaagcagagcagcaccccagagcagcccctcagcgacacctcttcagtaaagcaaggctgaacacagaggggctggcttcagtgtggatgtcaggtacagaaggcagctcgaggagctactctggcgttcttgcttactggtattcttacctcgaactggccaactcctacttaaactgcaggccatggctttaatgtcctgtcattcagaggctgtcccttacccaaagccaggttagcatcccctgactgacacttctccctgcaacacgtttcagaaggccctgtagtcgtccacttccctgtctctctccccaagctcctgagctccatgtggtctgggaatatgtgtgttgctcacttcctagcacagtcagtgctaataactgactgtagaggggacacagtcgaaaagccacatggggatcagagtcatccttacacagttgacacctcccaaacccagatgagctgtgtccaagtgcaggtcagaggaattttctgccgaagtctctgagaaagggtttatttacattttgaggttgcaggggaggagatgaggccatcaaaccaaagctgaggaagagggatcctaggatgcaccgagcagctccgggggcgcctgacagcacctgggaaagatggcttctccactggcttgttggcgtcaccctccagaggggcatcaggaaatgtcctgggaaccaggcaaaccagtgagcattaacccttagaagtgcttggcatgggtgacacccaccatctgtaaacacgacttctcccaaggagtgacgcagaacaggatgtctgagggaggcactccgactccagccttcagagatcgccagggtggcacctggtgacgacaggctgatgcttgggtgccccagaaaaggtcatgtgtgtgaatgggggccccaaagccaacgcttcatccctgacagcctggtgcatttagaggggaactttttgtcccttggcaaggtgggtggaatttcaggttcatagggcaagggtattttagctttaatagatattgtcaaacagttttccaaagtcattgtacacactctgtgattctacttatgtaaagtttaaaaacaggcaaatcaaatctatggtgttcgaagtcaagacagtagttacccttgtgggggctgcaactggtacagagtgtaaggggggactgtaggatggtctatttcttgatctgggtgtgttcgcttttggaaaagtccttgagttgcatttataatgtgtgaacttttctgtatgttacactttaattgaatgtacaaaaagtctcaggaggcctcagaccactggaagcggacacaactaacccctctgagagcctccaatccaagatggacatatgtccccttggaagtatgcagaagcaggtgaagactcctaagccggatattcccaaatccccccagtagccgcagcttcagcagctgcttatggtcctccctacaccctctcttccccagacagcccccaaacatctggctgcatttgacttgctctctccctgtcccacctctggatttagtccatgttctccaccctccccactgtcagcaatgtagacaagacaaacgcttagttcacgtgcccacctactgcgtgccatgcacggggctggtcattgtgggtggcaaatgtgagcaacacacgaagcctcaaggagcagaaagggacacaaatcacttcagcgtaaggtaatttgtgataaatgtcatgtaacttgcagcccctggccccctcctacagatggtgtctaagaataaaccccactaacatgtgactcctctgttctagcccagctgtttgggttgcaagaaagagactcactccagttgcgtcataggatggagttttattgggaggacattctggacgggctcccagcaagagtctggcaatggagcatgaaaatgaatgagcctggaatgagggagggtgcggcctcggctacacaaagttcacccggcccccaactgcctcccagcgtgttagctcctgtggcaactccccacttctctctctgtttttcaacccaaatcctagagcagagggctcttcgctcctcacaccatctccaccaggctgcagggggagtcactagcccactcaagagctctctcctgttggtccctgattcgcaggggcacgtcattccttcttggtaactcattctctttaacagcccgacacagggtctccaggaaaaggacaggagctcacacagtcatgaacagagatgcatctggagcaggaataaggatgctgacaacatctgtgtctgccctccactcttgtaagtgccagtaaggtagtacccagtccctgggggtagggggggtggtggtgggaatgagggcatctcttctttcaggggccaaatctggcacaaggatgtctgcttaggcaactccactgcctggcatgctctctccctaggaaaacaccaaaccttttttatttcctcagtcttagtgtgagagtgatcacctcttccaggaagcccacaaccagaatggccaggggcacaccctggccctcagtagatgggcactgagacaaaacatggcctgctggtggcattcccaacaatgtcaaaagtctcagggatgagctcacagttgggagtcctttggaagtccaattccaaatgtcctctggactcaaaataaaaactacatttcccagtctcccttgtagcttgcagtagccatgtgacaatactccagccaatgggaagtgagtggaaaagtcctgtgcagcttctgagttgcgtccaccatgggcatgggtgtgcttctacctccactcttccttcttctggctggagggcatgtggacaaagcggggccatggtgagcctcacaatcaaaggcatcattttagggatagagaaaaggaagatagaaggatcctgagccccaacatcataaaccatcgtaacagccagactagcgtgggagagaaaaataaaattctatcatgttgaagtcactgtatttggtctcttgttagagcaggctgactgatactctgttgaatacagaatgccttggtgagcctctgaggatgcaggatgctgcaatccaaccaaagtccagggtgactttcgaggagaaggatgtgaaagggcagggcttcctttgagggagaaatggagagaagggaggagatccagagaagagagagcgagatcagctcagtgctcttcagtgtcctccagggtcaacaccctcctgggcagacctctcaccacccatttttgggggggcaagaggcttggggccaggtcataaaaagtcagatgtcacagagctgttttcgtagaggcgggcaggactcaacactgcctcattcacattcataggctggagtcatcacagagtctggccactttctcctccggagggcaggcaacaccactggtcagcccaggggtggggcacaggttgaggtctcacatgtggctgcatcaggatcaatgagcttcccacagagaaaaccctggtcacaggaggcctctgggagccagggcaggcccccaggcccccagcctcaggtggggtcccaagaggattggtgggagtgtgcatggctggaagtgtgacttggagccctggcctaccaggtgaatcagctgcagaagggacacaccacaggggaggagaaactgactttcacttctgcccaggtgacgggcaggcttgggagtggggaagaggcctcagaacaaaggtgggggagtcagaacatacgtgtcttggaaatggaggggatgctgccaagtccagaaagtctcccctagtcctctaagccagccgccagctgacacagggattccctctgccccgagcagaacagggctctcctatctcctgaggggctcctggttccctgcacactctccccaacctccccttggtcccaggcaccatcctctaaatgacagcagtctccagatgccagttggcactgtgggttagctgggtattatatctgcccgtgtgtgttgaatggatacctgcgtggtctctttctgaaaagccatctccaccgaattctcgcccatgctctgcttacaaacacgcctccttctgcaaggcccggagtggactcagagagcctctcccctccccacctcctgcccccattcctctcccgctccccacaggctggctccagaccacaagagcgtgggaacctcatggagagtgggtgcttcctctgtcttcctccctagcccttgctttagcacagccagggcagagaggaggaaagaaggaaactacagcaaggaggatttagggacaattctagaagggatttccaatttggaggggcagtctgggaagtaggcagcctgtgaacttaataggaggtcctaaaggacctgggggaatttggggagaagacgaggtgggctgccttaccccaatcttctggcccactcccagctccagacccacctccaggtgtagcaggcccccaggcccaacagcaggagcaggaggcccaccagcagtcccaggacccagagcagctgctggaactgatgcaggcggtgcagggctggaacacagagcgggactcagagcagccacagctgcaggcccccagtgcttccttccaaggggacccacccaagatgacaccttctaacttttgctttatcgctgaagctctccacacaagggtgccccgagcaatcagtttagagtcgacaggcaaatcatgctgcagcaggagggatacagggaaggaagcctttgggcctaagagctgggcttttgtgcagagtggcctgggagatggagccccctcccctcacctctgaccccaaagtaggtggaggtagaggcagagcccaggacatttgaggcagaacagatgtattccccttggtcgctgggcctcagcgcctcgatgtccaccctcagggcattgggggaagccaggacatggatgtgtggctctgcaggagcaccctggggctgactggaggccaccagtcgactgccaaggtggagagtcaggctggcgagcggctcgctgtccactcggcaatccaggatgccccggaggccaccctcaggctccacgaagaccatcatggtgggcgtcttgggagggtctgtggggaggaagggagtgtggtgattgcagaaaatgaccacaaatttctccctccctgtacccatgcacagtgactttgctgctcctcccattaagaggcagaacctatttcctcactccttgaatctgtgactcagtttgaccaattgaagaaagaagtgatgttgtgcaacttcagagccagacctcaagaggccttgtagcttctgctctacttgttggaacacaacaatgtgggaagcccaggccagcctgctgggcacatgtggcccaactggcagccaggagcgtgcagtcatctgagaccactgggcaactgcagccacatgaggtgtgaacaacagaagaactccccagctgagcccagcccacagaattgagcagataacatgacttttgtttgaagcagtaagttttgttgtggtcaatagcaatagctgactgatacagtgtaggccttggtgaaggctggagtgggagaccaagactgatgagggcttatcgtgtgccagacacactcagcacgctttatttcttttggttttttctcttcttttcttttcttttttttttttttttttttgagacagagtctcagtctgtcacccaggctggagtgcgttgtggtgatcttggctcactgcaacctccacctcccaggctcaagtgagtctcctgcctcagcctcctgaatagctgggattacaggtgtgtgccttcatgccgtgctaattcttgtatttttagtagagacggagtttcgccatattgaccaggctagtcttgaactcctgacctcaagtgatccgcccacctcagcctctcaaagtgctgggattacaggcataagcccctgcaccagccatttatttcttatacaacttgcaggatgaggcgaatgatgtattccccattttacaaggtcacccaaccagacagcgggagagccaggattccaacccaggactcaatatccgtgaagccttcactcttggcagacagaagaggaggaagaactgaggtgggcgtgtgagatgtggggagactaaggcggaggagggggttcacactcacagagcacacggagcatgactggagcagaggcagcagccccagtggggagctcagccaggcagtggtacatcccagcttgagcacgagccacgtgggtgaaggcgagagtgggcacaggctccgcgtgcagccgccggtcattccagaaccatgcaaaggtggagttgcccacaggcccagggccacccaggaggcggcagctgaggttcagggcagcgccctcaggcacgtccaggccaggctctgccaccacgcgtgcacctgcgggcggaggatagagagatgattggggatctgtaggccttgggggctggagcctctgggtgggacctctgaggggcctctgcacattgtgggaaggtctcagtctgacttggtatagggctttccaaggtggaaccatgccccctccagtgggagctgtgccccctccaccagattaggcttctcaaggcagagctttctcagaccagacagcccgctctagtgggagctccagcccctctcgtggacttggacctgagagcatcagagcccctcctcctcccctctcttccactcaccttctacctgcaaccgcccgatggtgctgattgagcccagcaagttttgggctgtgcaaacataggtgtcatcacctgcaggcacatcttgcacctgcagccgtagggcgtttcgggccacctggacatggcctgtccctgatgccaagctgtggaccccgctgctcgtggccagcaccttgccatcatgagatagggccagctcagcaggtggctcactgtccacagtgcactgtatcacagccatggatctggccctggagtcccggaaggaggacaggacagcgtcctgaggggcatctgtgggcaggcagggcacagatggggaccttgcttaggcaccctgtactgctcattgcctagctgcctggggttggtcaggctgaggcctctggaccaatggccacttcctagaagtgacaccttcccaggagtcctgtgggcagagtgctccaggcagggcttataggactgtctctttctctagcacctatgtcctctttccccaactgcccattcctgggcccactgcagtcctttgcccactcccaccagagcccagaagcaccctccaccaccctcctcctgggcagccctggccaaggaccctctgctcacagaggacttgcagggcagcaggacgggagctgcgggtgccctgggcatcctgggcctggcaagagtaggcgcctgcatgagcccgcgtggccaccaggaatgagagtgaggcagctggaccctcctgcagccaacgaccgttgtggtaccaagtatagagtgtgggtgcgtgggcagcagggtccgcacaggtcactgtgatgggggcaccttcaggcacggcagcctccggagccaggatcacccgcacacctgtgccaaggaggccaggatcagccgggcccagccggacaccacagtgggcttccatcccagtgggcttggcctaggccctgccttaccctccagccgcagctccagggacgtgttggcctggcccagagggctgcgggcagagcagctgtagaaaccctcatccctgggctgtggccctcgcagctccaggcgcagggtgttggggacagaggctgctgtcgaggaggccaagaggcgaccggcgtggctgagggccagctgggcgggcgggcggctgtccacagtgcacagtaccagggccagctgcccgccatggctctccaggaggtaggtcaggcgcaggttgcggggcgcgtctgcagggcatgagaggcttacacggagtcacaggcggcagcctgcaggaggccagtttgcaggtggcattcaaactcaggagggccctggctccccacccctatggctcccaaccacccagtctgaggcactgctcctctgcccagacaggactcaccccagcgccccctactcttaatgctccttgcccagtgctccactagctactgggtaccgagttccccactggacacctgtcgggttccggccatgccgtgatccctgtgggcttctgtcattcctgtgagtcccaccctgcatccagccagactcacagaggacgtccaaggtgataggtctggagaggcggggtgcccgaccaggggggcccacaccgcagcggtaggaggtggcatccctgactgtgacgttgggcagggggatggagtgggcatccaggcgctgctgcccatcctggtaccatgtgtaggtgagctgggccgggtgagtggtccacacaaggcaggtcaggttcaccagctgcccctcccgcacggtagccccgggccacacctgcacattcacagctggggagagggagggcacaggacaccagtgagggtcttgaggctgtgtacagcaggccacctgtctccatgtgggtgagctactcctactgctggggagccccttggcctttgggagccttgggtggcccacctataaatgtggcttagaactcaagcttgggaatagcccactggctcctgagtggtcccaagtagagttccacagagtcctggaaagccctgagcccctccctgaccctggctgggggcaaatggggagaccacagacctcccctcccctgctgcactttctcctgaaattccctgtttagttttatccactgggcatacttgtaaaatttggtgtttataaaaggttctcctaagaaactcaagtctggagtccaccatcgttaggaagggtgggaatggggactattcccgtgcccccaggcttctagaaccctgtcccacctcctctcctcttttaaaggacacacacacacacacacacacacacacacacacacacacacactgaccttgagcgtcgaagtcagctgaggccgaggcctggcccagggtgttggaggcctcacagatatagacaccctcatcctccagcatagccccgtggatctccagacgcagtgtgttgggggccacagccacatgcagcctgggagagctgccttcgggtccccccacaccttgtagggtggaggccacaaggcgatccccgtggagcagccgcagctgggccggagggtcactgtccacacggcacaggaggaggcccagtcgtccaggacctgtgtccatcagggtagtgagtgtgacgtggcgtggggcatctacacagggtggggagtggtcaggacccagccaggggggtccctcatccagggctctgtcatgtgacacagcccaggactgggggactgcattccttcccccacacctggatgggacaaaagtccttacaggacacgtggaggctgatgggtgcagctaggctcgtggtggctgagcctggggcctgggcttggcaatgataggccccagcttgtgtcaaagttatggctgcaaagcggagcgtggccgaggtcgactcctggaggggctggccatcccgataccaacgatatgaggtcccctctgggactcctgtgggtacctggcagctcaggaccacagcctggccctcttggagctcaggtgatggtgacacctggacccaggctcctgcaggggaaaaccaagagcaggtgagggctctccaccacacctctcacagtctgggaccatgtgcgtgtccacctaggactacacaacccaaccccatgtgctggagccgcagccccttccagaccaccgccagggcccactcagccctatgccttggcccctcctgctccccactctcctgcacaggctccatcctggcatttacctcccaccacctggtgggatggttttactgtgtcagtcatatctcgcctaccaaagcgtaagccttatgggcactggacttgtgtgacctgtaaccccagttccaggcactgtgcctgcatgtgttagctcctgacaaatgttggttcaacccataaagtactgaaaagggagggatttagatcatcccagggccattgtcctaactaggatctggactcagcatgggtgaccgagggcttggaagggaatgttagcagctatgttatcttgagcgctctcgccaggccagccctgcatccagactccaggccacaagagcacaggacctggggaccaggcaaagggcagctcctcagtggcccctgggtgtgaagcaggagaaccccaggttgcagggagtgagatggagggacagctggaatgctaagcaagaacacagaccatgcctgagaccacagctagcagggtcacgcaactggggaaaagctcctaaaactcaaaccttcaatttcttcctgcgaaataggtatgctaagtaagaaaagatacacagaaccaaggctcaaagtaaatgtcccaaaattggtgctgtcggtcaccgtggtggaagaaacactcatgttcctttgaccttcgcattatgggcattatctttcctttttcctggtttatttgtttagggagaattttttttttttaagacagagtctcactctgtcatccaggctagagtgaagtacaattttggctcactgcaacctccatctcccaggttcaaacgattcgcctgccttagcctcccgagtaactgggattacaaatgcccaccaccacacctagctaatttttgtatttttagtagagtgttggccaggctggtcttgaactcctgacctcaagtgatctgcccgcctcagcctcccaaagtgctgggattacaggcatgagccatggtgcccggcctgagggaggatttttatagagactatatatatatacacacacacacacacacacacacacacacacacacacacacacgtatgtgtatataaataaagaatatgaatatattatgcataagaatacacataatgtatatttgtatgtgtatatactcatacataaacatatatgcataatataaatatacatatgtatgagaatgtttactatgtaacaaaattatatgatcgcaaaacaagttatttcagaaagtgctcccaccaagcaagccactttgtgaattgccattgcctcctagtggctgctgcagttattacaggtgaaaatatctagctggaggcaaagggaggccttctgctggctggctggaaactgcccttaccaggactgaaaaagacatatttctgcacaaaattcacagcacccagcccaaagtctgaccagagtagtccccagggcatcagagtctaccatatcttcagaagactgacactcacctcggacctggaagaagagtgaggtgttggatgatccaagaacatttgtggcctcacagcggtagctgccagagtccccaaggcccagttctcggacctctaacttcagggagttggcctcagccttagcctggaaccgaccatgggatgggacctggggacccaggctggtggccaggaggtgctccccatggaacaaggccagcaaggccagggggcggctgtccacagtgcagatgaacagagccatgtggccctggcccatgtctaggagggctgacagctttggacggtccgggggatctgcaggaacagagggagctgaggccacccagcctagctcaccacattacaactgccacactatgtcccccacctcctgccacacacacagcctaagccatgctctttcctccccaaaccccagcccggcccctgcttctccccagaccacccctccaccctccaagtccccaggctgcccatgccagtcacccacagaggacactcaggagcacgggagtggagagctgggcaccagcctcagtcaggatgcggcaggcgtaaagggcagcatcagttctggccacgggcagcagtgtcacggtctccaggggaccctgggcccacagcaccccatttcggaaccaggagaagttagcagggctgccagcagcttcccggctcacgttgcaagtcaagttggcttctgtgccctcctgaagtgtgtgtgatggtgcaatggccaggacagtggctggagagcaggcggcacagcttactgaccacccccggcccccaggagccaggggtccagccgcccagcctggaaggagcaggaaggaggccccctggggtgcccacagggttggggagaaaagcaagctagctcacagggcagcctaggagaagtgggccctggggactgtgggggccctgggcagagagggttgcagtacagggaaggaggacaaggcagcccaactgtcccagctggggagtccttcctcagagaccagctgtgttgcctatccccggcctgaacagagatcatgggaaggagcagctccgctcttcctgaatgtggaggagggcagcgaagggcccccactgctgcacagagtggttttgcctgattagatcctcctcggagcagaacagtccagagctccagcccctgcccccaggccacccattccctgcctgactcaccctggccattgaaggtggctgaggtggaggcgttgcccagggcattgctggcctcacagaggtacaagccctcctcttccagcaaagggttgtgaatctccacacgcagcaagttgggggctttggtgaccttcatgcgtggggaacagcccccacaggtgctgcagccaccccctgatggcagggaagtggccacaacacggtccttgtggagcagctgcagcctggcgggggggtcgctgtccacacggcacaaaaggaggcctcgccgtccagccccggccccagcggcatcaaggtccagcctggtggtgaatgttggttgtcgaggggggtctgcagggaggaagaacatgggcactcatcccacggatgctccagggccccacaagcctggctaggctcccagaatacactggacaaaggcagatcccgaaggctgccccaagcagctgtgcagactgtgcactgcacaagggcagccagaccagggtgggtggaactcaagctaggctcatgctccccaagctaagctgtgccctgatgcctactgagtcttcccagaagaaggagcaccattttctagctcagacaaaggtgctgtatgggctggctgccaccttgcataagtcccatctgctgctgagcagggtgcccacctagcaatccctgggggtcatgctgtctgctcccaccctgcacccaatgccttaccacggagcctcccctcaagatctgccctgcagcttatctgtgatctccaagccagctcctgtgatactagcgaatgcatagaatggcctcttcctgaaggcttctggagacagttggccaaaagagagcaatcagccaaggcccccatgtgatctcactcagacaccagaaaactttgcagaggcagctgccctctgacagttccgggacgtggctccggcacacatgtgtcaggggaagcctctggaatcaggcccttggtcttacacggagcctccccgcttggcggatgcagaggggctgtctgtccctgtgctgaggggctttgccttggtgtctatgggagcccagcacagcctgtggggcatgtgcacaaatagaggagtgtggtgtcccagggctcacccatgaggacaaagcatggatgtggtgcccatgctgagcttgaggctgggacagaggagcccacagggctggcattggagggtaaagacatggggaggtagagaaggccccagcctcatttcctaaacttcagccccacgtggaaagacccactgtggccaggtctgtgacccagccctcccgatatagaccttggggcagggcaggacagggctggggggcctagaggaggtggggtggctggtgggtcccggcaggaggctgcaacaggtggtggctgctcacagagcacagtgagaacagctggcgaagaggggccactggcactgtggccgtcccgggcccggcagtggtatgagccggcgtcagtgctggaggccgcggggagcaggaggctgctgccgggaccctcgtgaagcagggctccattcaggtaccaggagaagcgggcatcaggtgtggggcttaggccgcttctgcagctcagtgtcactgcctgtccttccaccacctcggctgccgggctgatgaggagacgggcggctgcggggagaggaagaggctgggaagggtccctcctctcaaccccacatgctgccctatatagaaagccttccagggttctcctttccccacactactgtaggtagctctgggctttgtggtgcatcaggagggtttgccctttaatgccaaatcagatctattggtagtagatggctgcagcatggttgaagattcagagccagagcccaggcttatgtccaacacctggcttggctgggcatggtagctcatgcctgtaatcttagcactttgggagactgaggcagaaggatcgtttgaggccaggagttccagaccagcctaggcaacatagtgagactccatctcaaacaatttttttttttttgagactgagtctcactctgtctcccaggctggagtgcagtggtgtgatcctggctcactgcaacctctgcctcccaggttcaagcgattctcctgcctcagcctcccaagtagctgggattacaggtgtgccaccacacccagctaatgttatacatgtagtagagatagggtttcaccatgttagccaggcagatctcgaactcccgacctctggtgatccacccgcctcagcctcccaaagtgctgggattacaggtgtgagccactgtggccagcttttttttttttttttttttttttttttgagacggagtcttgctccgtcagccaggctggagtgcagtggcgcaatctgagctcgctacaacctctgcctcccaggttcaagcaattatcctgcctcagcctccctagtagctgggaccacaggtgtgcgccaccacacccggctaatttttgtatttttagtggagatggggtttcaccacgttggccaggctgatctcaagctcctgacctcaggtgatctgcctgcctcggcctcccaaagtgctggaattacaggcatgagccaccatgcctggccacaattttttttttttaattagctgggtgtggtgacatgggccgtagtctcagctatttgggaggctgagatgggaggatggcttgagcccaggagtttgaggctgcagtgagccatgaacataccattgcactccggcctgggcaacagagcaacaccctatctcaaaaaacaaaaagaaaaacctggcttgatcaattagctaccatgccctcaggaggagggaaggacagtgcacataccgaaagttggaagaccgtacttttcttttttctttttcttttttttttttttttttgagacagagtctcactcttgtcacccaggctggagtgcagtggcgctatcttggcccactacaacttccacctcctgggttcaagcgattctcctgccttagcctcccaggtagctgggactacaggaactcaccaccatgcccagctaatttttgtatttttagtagagatgggatttcaccatgttggccaggatggtctcgatctcttgacctcgtgatccacccacctcggcctcccaaagtgctgggattacaggagtgagccaccgcgcccagccagaagaccctacttttctatttggcttcccacatctgactgctagcatagagcctgctcccagagtttcataattaaaaaacaatgaatgcttctgagggactctccaagtttagggtcagggtaggtgcaaaaggaatgatgtgacctgttgtgtttccctttttcccttgacttccaggaagctctgccgttgggtcactgcacagcccctgtcttttatgtggcgtagccagttagctcagtcctgcggttgagtccactagacttctagaaggaacagactggagcaggctcctcctcaggctccctccactctccctggctgccagtgcccatcttaccattggcatggaagtccagggtggaggttgcatttccaagggagttggtggctgagcacttgtactccccactgtcagtttcctccaggtctcggatctccaggcgcagggagttgggaccagaggtaccactgaagcgtgggctgtgatcactgtccccggaggtggaggccaggatatgacccccatgtgacagcaccagtgtggccaggggctcactgaccacagagcagtgaaggatgcccacaagtcccgcctgggtctccaggaaggctgtcaggactggagtgagaggcgggtctgcgtggagacgagaggtgggcctgtcaccctcagacaagggcattccctggataccctgatacaacccgtgacctctgcaccgctttgtcccacactgccctgcgagaagggggtgatcccaaagtgcctgagtgcctgctgaatacacttttgtccttggctgggctgggtacgtcactctgttgtcccagtcagtgttcaaggccaccctgcaagtggggataaaagccccacttcaaagatgagaaaactaatagagagacctggtgagggacagcacctcagccaggtgaccaaagtgagcatcatcagcaatgggacaagctgacacagagtgcctcctgacagggcagagcagcatgtccgcggccttcccacccagagggcatggtctcaggccattcaagtcaggcaaatgtggagtgagggacctcctcacaacagcaggcctgcactctgcaagtttcaacgtcaagaatgacaaggaaagactgaggaacagtctcagactaacggagaatgaagagacgcaacgacccaatgcaatatgtgaactgtgattgtattctggaccagaaaaaaaatggctacaaaagacagtattaggtccactggtaaaatgtgaatatagattatagcttagataacagtcttctatcagccaggtgtagtggctcatacctgtaatcccagtactttgggaggccgaggtaggtggatcacgtgaggtcaggagttcaagaccatcctggccaacatggtgaaaccctgtctctacaaaaacacaaaaattagccaggcaggatggtgggtgcctgtaatcccagctactcaggaggctgaggcaggaggagaatcgcttgagccccagaggcggacattgcagtgagccaagatagcaccattgcactccagcctgggcaacacagtgagactccatctcaaaaaaaaaaaaaagtcttctatcaatgctaattttcctgatcatgatcattgcattgtggatatgtaagagaatgatcttaggaatttagcggtaaagaggcctcatgtctgcaacttcaggaatataaatatgtaggtagatacataaataacatatgcatatgtatatagagtgtccatatatgtataagtgcacatgtccacatagagtggatgtgcatacacacaagtgcacctgtatatatgcaagtctatatccatacatttatatgtatgtatgtgcgtgtgtgtgtctgtgtagacagcgagaaagattaagcaaatgtgccaaaatgttaataaatgggaaatctaggttaaaggtatataatcattatttgtctttctctgcaacttttcataagtttaaacttccaaatataaaataggagggaactagggaggtcaaagcattgcccaggtgtgcagagctgggacatgagctcaaggccacctccaggtaagtggccttgaagttcccatgcccaggaccccaacccttccttggggctccaccatctggtcctgctctgactgtgtccagtgccaccccaccctggcctcttacggttgactaccacgctgacagggcccgagcgctcgctgccatggacgttctgcacctcacagaagtagaagccagtatcagccctagtggccaagtgcagccggagggtatgggagtgggcatcctccagcaggacatggttcttgtaccagctgtagcggagatcactgggtgcctcgttgggtgtgttgcagactagtgtcactgtctggttctccaggatgggacctgctgggctcacctggacctcagccactgcaagggcagcatagggagtgctggggggtcccagccaacttccagccccagccccatacagtccccgggtctcagccaatcagcctcaatctctgcacatcctacaccacccgcctgctgctacaggggagcctcctggagtgctctgcatctctttgtcctgctcaccaggatccccctgacaccccacccttcgtgggggtattgtcacctgtctcatggtaaggcagggctgggactccacacctgcatccaggaagcactgggttatgccagttgctggccctgccctgccctgtctcccctccgtccctgaggcctgagctcctccctattctctggccaatacgcaaccttcccaagaactcactgaagatgtggaggctgatggggggtgagaccaaagagcccacgccgttctcagcttggcaggtgtagacgccagcatcgctccaggctgcctggggcaggtgcagcacaccagtcttggtttggaggcgtaccccatccttgagccacttaatggaactgactgcagggtagctgctgttcacctggcaggtgagtgtgaccagctcacctggaaggatgttcctccccgaggggctgaggaggatcttcacacccctgggggcatctgcaagtcacagtagggggtattgggtaaggtgcttggggagggcagaggatggcacacttcttcttgcccccctttaaaagctcagtcctaaggaagtatgcccagataaaacagcagtccccttcaatcctcacccagggcatgctctgtctgcccatgtccgtccctttcccgccccctgcgcctgatgcattcctatgcccattgagagctgatcatgtgacgcttggccagcgtccagcctaccccaccagctgtagttttctgcttcccaatgctgtccattgctcctgctctatttgggatgagctccacacacagggtcatgggtaccccactgctagctttagtggcctgtggaagctattggtaagggaccaactacctagtgggagggggccaaaggcagcatcaaactagctctgaaaatagttacccagtttgtaagcaagaggccaacaacacaaagaactgcatttccttaaatcttgttccaaagcctctctctgtgtattcaagtgttttattcttatttttttagaaagaggatctggctcagtcagccaagctggagtgcagaggcacaatcatagctcactgcagcctgaaactcctgggctcaagtgatcctcctgcctcagcctcccgaggaactgggactacaggtgcaagccaccacatccagctaatttttgttttttaatttttttgtagagacagggtctcactatgttgcccaggctggtctcgaactccttgcctcaagcaattctcctgccttggcctcccaaagcactggggttacaggtgtgagccactgtatccggcctcaagtgtttaatttgtgccaggcactcttctaaatccttgacctgggtcatctcctttatttgtttttgttgttgttgttgttttttgtttgagacacagtctcactctgtcacccaggctggagtgcagtggcacaatcacaactcaatacaactccacccccggggttcaagcaatcctagtgcctcagcctccttagtaactgggattacaggcatatgccaccacacccggctaatttttgtatttttagtagagacgaggtttcaccatgttggccaagctggtcttgaatccctggcctcaagtgatccacccgcctcagcctcccaaagtgatgggattacaggcataagccaccgcgcccggcccatctcctttaatttttatcataaatctgtgagacaggaaccatctattgttatcttcgtcatagactgagaaaacagagccaggcaggaaagggataaatcccacctccccagcatcctccagcacggggtaaatcccggggcacttgctgccaaccctgaagctgcatgggagctcctgttccccaaccctttgtgtctccctttctctgcccagcctggcctcaaggacaagccccttcgaagcagtaggaggttgagggaaccatttaacgaagtccttggggctcagcagtgtctctccacttgccctaccctggaggcccaggagcagcctggttttgcatcaggagcaagggttccgtttctgtgggctggagaggggctggtttctgtcaggagcaacagatgcgctcagccacaagggtgtgtccccagacaactcacacttcacttggaggtgaatctcgctctgagccctgtgattggccacggagagctggcagcgcaggatccggccgtggtcctgccaggacatggccatgtggagggtctccaggtggccgacgccggtgggctcaaacttctggctgttgaaggtgacagagcgagcagggtcctggccttgccactgcagtctgacctgctcctgcaggcatacgtagggagtggagcagttgaagtccacctctgtgccctcgagaagctccaccggggaggcaatggtgggcaccctgggctcctctgaggacagagacagcagtgctcaggacccgcttttgccacccctgagatccctcgccctggaaaccccagctgaggagagagccctggggagggggcttttaggggaaggaaagacgctttctactccagccccacttgggtgaatacaagggagaaccaggcctgggcctacgccggggctggaacagaggctgagactggctggggttagattcaggacaagggctggggctgagagccaaggggtccagaagcagcttgggaatccctcccggggggcagccaggccaccccacttatcacctgttactgtgaccaaggtgcctttcacatctgaccagcggttgacctcactgatctcgaagcggaagttgtaggaaccagagtcctcgggctgcaggtccttcagcagcaggttgcacaccctgtgctcggggttccccatgaactcggtgcggccgcggaagcgggcctccaccagcttggggtccgccgagtggctcaccacctgccgctggcccgagtagtcgtagtaccagatggccgtgatgccgtcgggcacctccacgtcggcagggaagctgaagatgcaggggataagcaggcaagaccccttcacaccctgcacgtcctggggactggagacgccccatgaggcctggcctgggggaagaacggcagggggacagaggggagggtgatacaggcctcagggtgccacagagccctgcgacctgccccagagaaggtgccccagctgggctcccaaattctgccctgccccgagaaatgcacacttagagcagcccttctcagtgccccaggggtcagtccactgcccgaggctgctcagaggtttggtagggtggctcgaagacagatggcagcttcctgcccagctcctggccactgcccgattgggccctcccttgacctcagcaaccaaacatgtgacccaggcatagtctaatctgccaagggctggacttatgaaggctggaccctggaagacaggcactaggcccgcaaagcttacctgctgggaagaatgaggccaggaggagaagcttgggcaagaagcccatagcaggttcttgtgctgctcctgttgcctaagagggtggtgcgcactgcgctggctgggctcacaggggcctccagggacacctctgggcactttagccccagcacctgctagaagtccgagcctgtgtccccacctcctctgctggccaacccaataagagggcagggctcttaaagacctctgagtcagacaccagcagagagccaggaggccacgttcccagctcaggctgtgcccaggaatgccctcacttggtggcctgcctcagaaaagcctgtgtgtcccttggccctatcccaagttctgctttcccagcccctcaaggataccaccctaaggcagatgaacagctgtttctccctcttccccacttccctgccccctccccaccacccaaaccaacaggaactggagcccagagatgcccagttactcactccaagacacccagctagaatgatggtttcttcctgaggcttgtctcctaccacctgccttactaactatagaccataatggggctttactgaacttgccgaagtgctgcctttaacagtcactcccctgctcaaaaaccttctgtggctccccattgcccgtgagatgtgaaaagtcctcatttcctgcccccagctctgtccccattccctgctctccgcagacccactctgggggcagttctgtctgctcaagggctccctagctgcccagctctatctccaccacagataatctttgcctgctgaaaccttattcaaccttcaaggagcagcatgaatttggcttccagctggaacttctcctttgaggttcctgtagctaccccagagctatctctattttccttgccttgttttttacagcttgtgagagcccatttctcaggacctagaactgaagatatgtgccccatagcagtgcggagcctaccaggcattcagcaaaccccttagtgactaagagaggggtgaggtctttaggggttcagagctgaggttcagagttggagtggggaggtggcaaggcaagtctaggtttgaaaggtagcatgagagcgctgtggaacacataccccacaaatatgagctcaatgtgtgcggagtgtaccatatccaaaaaggcaggccctcaaccatggagtgcccctggtcagggagtgtctaaggggtaccatagacctgagcccaaaaggaagagatgccagaaacacatataagtgaaactataaaactcttagaagaaaacaggtgaaaatcttcatgaccttggattaggcaatgctttcttaagtatgaaattaaaagcacgaaaaaaaaaaataggtaagttggacttcatcaaaatttaaaacttggccagacacagtggctcatgcctgtgaacccaacactttgggaggctgaggcaggaggatcactttagcccaggagttcaagacgagcctgggcaatactgcaagactctgtctctacgaaaaattaaaaaacaggcctgtggtcccagctactctggaggctgaggtgggaagatcgcttgagcccaggggaggggtcgaggctgcagtgagccatgattgcaccactgcactccagcctgggtgacagagcaagaccctgtctgaaaagaacaaacaacagctgggtgcggtggctcacgcctgtaatcccagcactttgggaggctgaggcatgcagatcacaaggtcaagagattgagaccatcctggccaacatggtgaaaccccgtctctactaaaaatacaaaattagctgggtgtggtggtgtgcacctctagtcctagctactcgggaggctgaggcaggagaatcacctgaacccaggaggcggaggtcacggtgagccaggatcacgccactgtactccagcctggtgacagagtgagactcttctgtctcaaaaaaaaaaaaaaaaaaaaaggatatgaatcaaaggacactatccagagagtgaataaaaggaggacaacccacagaatgggagaaaatatttgtaaatcatttatctgataaaggactaatatccagaatatataaagaattcctacaatgaacaacaacaaccaccaaaaaaaccatgaaatccaactccaaaaatgggcaaaacacttgaataaacatttcttcaaagaagatatataactggctgataaggacatgaaaagatgctcaacatcactaggcattaggaaatgcaaatcaaaaccaaaccacagtgagataccacttcacatccgttagaatggctattcacaaacaaacaaagcaacacagaaaacaataaatattggtgaggatgcgaagttgaaattcttgtgtattgctggtgggaatataaaatggttccgtcactgtggaaaacaattgggtcattcctcaaaaagtcaacataggattaccatatgatccagcaattccactcctaggtatatacccaaaagtactgaaaacagggactctaacagagtacaccaatgttcacggcagcactattccactaaaaggtggaaacaggtcaagtgtccatcagtgaatggatgtggataaacaaactgtggtatgtacatacaatggaatatcaatcggccataaggaggaatgaattctaacatatgtgaaccttgaaaacattatgctcagtgaaaccagccagacacaaaagggcaaatattgtagggttccaattacatgaaatatctagactacgtatattcagagactgaaagtagaataggatagaggtaaccaggggctgcagggagggggagctaatgtttaatgattgctgagtctctcagataatgaaaaagttctggaaatagtggtgatggttacacaatattgcaaatgtacctaatgtcatggggtgtacacttaaagacagttaaaacagtaaattttacattatgtatatttcaccacatacacacccatgttgccaactttgcaatcctccctggtcctaaatgctgacttggccaagtgaacgaggaggctggaagtggggacaggaaactcatgacctcccagctcccagcccatccgcctcaggggctgggctcagcagattccaatgactaccaggggtcacacctgggaagggggtgagccgaggcccagggccagtcaggctgaccaggtgggacttagcctgctgcagaaggcagaaggtgccccagcagggggcacagtacagggcgggattgggacaggaaggacaccgctccccaggggacccagccctctcgcaggctgctggagtggactgatctggccatttatggaggcccaagggctcatctccagttctctaggaagccctaggcctcctcctcttctgggaagatgcacccccagcctccacaccaggttcttggccactggagaatgatatagctggggccctgggacctggacacctcaccgtgaagaaaagcagcctgctgggcacactgggggtcagatgtgtccctggccacaggggatgtcagggtcagctctgctatggccagggcagctatcttgtcccagctcccctgttcctcccatttggggtcctgaaaagggcaatcgtgaacctgatggaagaatggtggggttctggacacagcgaccctggaacagggcgcgggggaggaccctttccaggaccactcccatcacataatgtagaggccacctatgcttagcccggccctaaccccaaaggggtcagccccaccggaatccagcctattggctcagcctgtcaccacaaaggccagcttcagcccagataactgttctggaaacagaaagagcagggaccgctcagaaaggagatctctgtccctgtttgaaagcctggagttgaggggacagtgccccgccccccgccccccgcaacttgggttgcagctgtggcctagtgagcacgcagcgccccctggtggtcgagggggaattgcgggtcccgggaaagggggcggtgtgccagcaacagggagcaggcagctctgcagccctgaaccatccctcccttgggtgactcttttggaaatcattgttccccagacaggaggttcctgaggttcatacttgggtctccaagtcttgggtgctctgaagacaggattttaaatccccactcctactattggtatgtgttgcatcagggtggcttgagctggcctggtacacagtgggcactcacgtttgctgcctgtttgagaccaagtgcctcaggaggtcttggagggttggctggggccccaagtccctgacctctgattccagaggccaagtttagctggggaagaaagggcagaggcagtttccctatggacagctaggcccgggtgtaggattcagtttctgtttcctgacaccaaggcttctccccaacttccccattgggctagagaaggaagaacacagggtgacatggccagctggaggttactggcccacagatagggagtcagggtacggatgggcaattcctggagcagattatggtcaaaatagtggaaatccccaatcacaggccaaatgtttaattctcagagccatagaatccataactaatgcttattggctttgacatgggcagtagaaatttcacacttcattccttaatctggctctaaatgcttctggctggagtgctcactctccaaactgtgctggacagcaccagaagccctcctgtggacggacgaagtggtgatggatgaagtggaattgtgctggggttagagtaaggagagattatcgttgggctgggctggggctgaggtcagggtgatgatcaggttggaattctggaagcaaattaaggctgggattggggtggacattgaggttgagtgatgggtgggggtaagggtgaaggttggagttgggtgcaggtgatggttaagatacggtggaggctgggttgagatgaggatgatagagtcggagttgtggttagggttgggatgatggatggttgggattagatgagatgagtaaatggttagggttggggtgcaagtgaggtgagggtgagataaggttggaataggggctggggctggggctgaggtagggtcagagcagggtgatggtcgggactgggattgggatgcaggttggaaggatttgggggtggtggaagggtttcagttgagtccatctttgattagtgtcttggacttgggttggtttggggtccaccactcgcacccagatggagcccccccccgacccctgcccctatcccgctcagccagtttcagcccagccccgctcctaatgctccactcaccctctgggcccagggaccagggacaggggtacctgctccacagaaggaagtggctgcggcggtgctggacctgcggagaggagaacaggaaggacggccaagagctcctggtgcagctggctccccagggctctgccggctcaagagagaaggatcccgtatcaggggctgcttcctctttcccaaagcctcagctctactgtccaacccagaggctggtcagggaggcagctgcaggccttgcgcaatgccaaaacgggaaagacctccataggggaaggccctcggcaaggccagggacttagggactccagcaagcagaagtgggaccgctgcaacgctggagcctccccaggcaaagtgagaaatggagtgggggactcccattcaccagccaaatccacaccccactctctctgagccccttagggaggctgggggaggtggaagggggcttcctgcacacagctctcctccctgacacctgagagggaggcgcgcccagcctggggtggggatgcactacacgatgcccagaccaaggcagttattctccaagccattcaggagcctccctgtaccactgagtactcttaagaacccccaagaggcagtcccgtgttgtgggggacataaggccccaatgtccaggacactcctcaggttttctctttcccctctcactgtcctgtacgttttttggtttttggcttcttgttgttggtttttttctttttgttttgtttttgcttttgagatggagtctcactctgtcgtccaggctggagtgcggtggcacaatctcagctcacggcaacctccacctcccgagttcaagtgatcatcttgcctcagcctctcgagtagctgggattacaggcatgcaccaccacatccggctaatttttgtatttttggtagagacggggtttcaccatgttgtccaggttggtcttgaactcctgaccccaagtaatctgcctgcctcggcctcccaaagttctgggattacagaggtgagccgctgcacccggccactatcctataccttcacccccaccttgggacaggaaaggaaagcccccaccacagctagttactgttacattactgcagcccatcttattgagggcctagtttgtgcaggcacttgacatgtgaagcagatgttaattgctccaagcaatcccagatacaagcaacaagactttttttggtttttgtttgtttgtttgtttttgttttttgagacagtgtcttgctctgtcgcccaggctggggtgtcctggtgcaatcttggctcactttggctcactgcagcttcgaaattctaggcttaagcaatcctcctgcttcagcctcctaagaagccgggattacaggcgcttgccactatgcacggctaattttttaagttattttgtagagacggagtctccctatgttcccaggctggtctcaaacacctgggctcaagtgactctcccacctcgacctcccaaagtgctggaactacaggcgtaagccaccactcctggccatttttttttttaatttcacaactcaaacttaattcaactcagtccctgcctacctgtactggtggggagctggccacagcaaattaggcccctgtaccctgagggcaaggccacgtgtgcaggtgtaaaaggatgtgaaaccctaaatcagggtggatccagaatcgcaggccatggtgccccaaagcagatgtctggtgacattccaccctgaaatgctcaggctacagagatattaggtctctatcactctgttcctctttatagctcctgtgtccactcctatgcttgggccatttcctctttcggccaaaacacaaagggttcatcccattacttcctccctcaacagctggtccggagacacccagctctaggcctgtggggttgtgacacatgggtaccaatccttcagtccactgggactctatacatccaccccttggcttcatggtgggaaagcatcccttgactttgaccttagtcatatgacttgctctggccaatggatgtgcacggacatgacacaaactaagtcttgaaacgtacttaagcagtttgcctttccccctgaatttctgtcattgccatgcaaggggcaggctccaactaacctgctggtccaaagaggatgacgaacacatgcagatgacctgaatcagacccatggattgaaacaaaactcagctgagcccagcctacgtccaccagaccagtcaacctgtggatgcgtgaatttattgctggatgctgctgagaattttgtggctacattagcaagatgatacaaggcctaagtcccagaacaacacacccagaacttgcttacctttccttagcatgaggagagcaaagacttgtctaccttgattagtcagggagcactgcttcctgtcatttccttgagtatacagcaaactaggtaaataaataaaaataactaggtaggctgggcacggtggctcacgcctgtaatctcagcactttaggaggccgaggtgggcagatcgcttgaggccaggagttcaagaccagcctggtcaacgtggcgaaaccctgtctctacgaaaaatacaaaaattagctgggcctggtggcaggcgcctgtagtctcagctactcaggaggctgaggcacgagaatcgattgaacccgggaggtggagattgcagtgagccgagatcacactactgcactccagcctggatgacagagcgagactctgcctcaaaatattttaaaaaatgtaatttctcagtaggtcacactgttacacattcatctaataacaattattctttttctttttttttttttttttttggagatagggtctcactgtcacccaggctggacaggctggagtgcaatggcacaatctcggctcactgcaactttgacctcctaggctcaagtgatcctcctgcctcagcctcccaagttgctgggactacaggtgagtaccaccagacgcagccaatttttgtatttttttgtagagatggggcttcaccatgttgcccaggctggtctcacactcctgagagttcccagtcaagtgatccacccaccttggcctcccaaagtgctgggattacaggtatgagccaccatacccagtgaattattctcgttccagataggaaaaccaagtcatagggaggttagagaatttgccaaagacaaaactttttggttgaaaaaaaataagttttgctacaagtatagaaaacaccaaataacggtgttttaaataaaatagaagtgtttcaccctctccctcaagtaagtgttggcatccaagatgatatgacaactccacaatcatgaaactagatccctttttattttttggctcagtcattgtcaatgggctgcttccagtcattgtccaaagtggctgcttgcgctccagccactgtatctgcattcgggaaagcaggatggaggaaaggacaaagtaggccatgccctctactttaagataactccttaaattgctgatgtggtgggtcactcctgcaataccagccactcaggaggctgaagcaggaggttcacttgaacccaggagcttgaggctgcagtgaactataattgtgtcactgcattccagcctgagtgacagagtgagatcttgtctcttaacaacaacaaaaaaggtaattctttaaattgtacactttattgctgcttatattccactagaagctagaagttagtcacatggtcttaagtctttattctaacaggtgatgtgcccgagtgaatcctgggggcccagtgctaagaagtagagggagatgaagccctgccctcctgtggagcaaccaggaccttaattcagtcattcattccttggacctacccagattccagcctcaggcccctgcccacctccttgccagtatctctgcagagcctcctgcctcccccagagggtgccagagccctctgcttcctcagccttcagacccacttgctgtgtctcaggtggggacaactcagtctatagaggcccaagggagatctttgagacccatactgtagtcagcttggggcagatgggagtcatgtagctcactggctaaaagcctaggtcctgcatagagctgggttcaaatccaggcatatccatcacctgctctacgatcccaggagtcactcaatgactcagaagctagagtcctctggaaagcttgtatgaagagttcccagggcctggaacatcaaaagagctctgagaatgttggctctgctgttgcttctcttattgggtatctgaaggccgtactctcctctctcctccacccgaggtgacctctactctcatagccacaccctggaccctcactcattcagtcacctcacccagtctgtcatctgtccctctggctctcctctcctgttcatgcatcttgtactcacccacttctgtccctgagtgtttcaaggctctggggttttcagagacactgaagggacctccctcctcaacacaaccacaaggtctaggtggaatgacccactaagggaccggctctgaagccagagacttccagggaaagtcaacaagcccaaggatgcccgttacaagaaagttaaaagacccatgtacatgtcctcccgttttattccctgctcagggtctgggcatacagtggaacacatgcagtccccaaaggacaccgtctgtacagagtcagatggagttaagaacatttagccggctgggcgcggtggctcacgcctgtagtcccagctactcgggaggctgaggcaggagaatggcgtgaacccgggaggcggagcttgcagtgagccgagatcgcgccgctgcactccagcctgggccacagagacagactctgtctcaaaaaaaaaaaaaaagaatatttagtctgggcacggaagcttatgcctgtaatcccagcactttgggaggctgaggtgggcggataacctgaggtcagaagtttgagaccagcctggccaacatggtgaaaccccatctctactaaaaatacaaaaattagccaggcatctgtaatcccagctactcaggaggctgaggcaggaaaatcacttgaacccaggaggtggcggtcacagtgagccaagattgtgccagtgcactccagcctgggtgacagagcaagactccatctaaacacacacacgcacacgcacacatatttaaaccaccacaccaacatctagttcaagatggtggactgagaacttgtctctgccattcctggcccatccaataccactgagagcacagtaagcaaagggaaaaggagacagaagggctgggaacaggatggctgggggatgggaagtatccactgcacaggattttgatttaattctagaagatagaaagagggaggatcacgttcaggaacagatgcgggtaaaggaaaccagagccaaagcacgctgagagaaagctgccccagaggccggagcagaagtggactctctacagggactcaatacaccccaaagggttggtagctggcacacgtacctctccacccccacatgtaacactgcagggcagaggaaataccctaggtgagttccagtatcaatcaacctgccctttgttcataaagatgaattggttaccaggtattaccagacaggtgaggaagaccaacacaaagagaaagatccagaaacaaacaggccaggcgcattggctcacgcctgtaatcccagcactctgggaggccaagatgggtggatcacctgaggtcaggagttcaagaccagccttgccaacatggtgaaaccctgtctctactaaaaatacaaaaattagctgggtgtggtggggttctcctgcctcagcctcccgagtagctgggaggctgaagtaggaggttcacttgaacccaggagctcgaggctgcattgaactatgattatgtcactacattccagcctgagtgacagagtgagatcttgtctcttaacaacaacaaaaaaggtaattctttaaattgcacactacattgctgcttatattccactggctagaggttagtcacatggtctcatgtctttattctaacaggtgatgtgcccgagtaaatcctggggccccagtgctaagaagtagggggagatgaagccctgccctcctgcttgaaccctagaggtagaggttgcagagagcggagatcatgccactgcactccagcctgggtgacagagtgagactccatctcaaaaaataaaaaataaataaataaataaataaataaataaataaataaaaacctacagcaaagaacaaagagctattgcaccgttactgtgggcctggcagtattatgacaacttttttttttttttttttttttggagatggagtatttttctgtcacccaggctggagtgcagtggctcaatctcagctcactgcaacctctgcctcctgggttcaagcgattctcctgcttcagactcccgagtacctggtattataggcacatcccaccacacccggctaatttttgtatttttagtagagaccgggttacaacatggtttcaccatgttggccagtctagtatcaaactcctgacctcaggtgatccacccgcctcagcctcccaaagtgccaggattacaggcatgagccaccacacccatccagtgttctgacaattttatacttattaactcatttagcaaccctctgagctagatgctattgttatctccacttacaggaaactgagtcacagaatggtacagtaaaataaccttgaccgaggtttacatggctggtaagtggcagagaaatgaaacttgaacccaggcaacctgtttcctgagttggactctgaactactcagccatactgcctatttatttatttatttatttacttacttacttatttattgagatggggtcagccgggcatggtggcttacgcctgtaatcccaccactttgggaggctgaggggaacagatcacttgaggccagcagttcgagaccagcctggacaacaaggagacagggtcttgctctgtcacccaggctggagtgcaatggtgcaatcatggttcactgcagcttcgacctcccaggctcaagcgatcctcccacctcagcctcccaagtagctgggaccataggcacccactatcatgtccagctaatcgaaaaaaaaaaattatatagagatgggggtctcactatattgcctagactggtctctaactcctgggcttaagcaatccacccaccttgccctcccaaagtactagaattacaggtgtgagccaccgtgcctgaccaatacttcctctttaataaactaaacaaaaagtgaacaaaccaatcccggagggaacagataattcaaggaatacaagaaaacttatgaaaagaaagattctagtgccaatatgttcataaaacaaaaaaaagcagctatgaaaagaaagcaaaaaataattcttggaaatttaaaatagaattgctgaaataaaaagaaattcaatagaaaagggtgtgaacatctgtggttttggtttccaaaattagttcactcttctttgggtaaaaataccctgattttcctgctgtattctcaagccctgtgggttggtggaattgacacctcttccatttactgcacgagagaagcatgttgctgctcacagttcattatgaggagccagcaggtaaagacaacactgagggcagatgaaagatatatactgaccctgggtccttgtggacatcattgagtccctggatcaagccttacctgaagctaaagggatgagtgcttctcactgttaatagccactgatcaaattccatagaaggactccaattatccttgcctcagttgtgtgtccagccctcggatgagctaccatcaagggaaactgccagaccttggtgacaagcccacccactgaaaatggggaaaaaacccaagtcacatcattgaactgaaagagctgcagttccccgaaagacaggagagaaggaatgctggataaacacaacaactacttcatctcactgcacctgcaaagaatccaggcacaggaaggtgggcacagcaagtagtctttctggttgacataacaaagacaacatctgtttcttctctcagaaagaagtaagaaaggcaattggagaaccagaaacgacaaaatctattccaggaccaaatagaaagaaaacacatctgaagcatgattttgaacaattagtggggtgtaagaaaagagaatccatttgaccttgacatctgaaacagcaaatattctccatcaaggcactttagggtagaggaaatgatactatgtttccttctcaaaggagctatctggttacgtggttctgcagtaaatgacgtttatacaatcataacatccgttggttttcaattttcagaatcaacctgaagataaagcatggaagatttcattataattaccgaacagcatgcaaatgttacaaactttgaccatgaaaatgtaaaaaagagcccaggcgcagtggctcacgcctgtaatcctagcactttcggaggccgaggcgggtggatcacaaggtcaggagtttgagaccagcttggccaacatagtgaaaccccatctctactaaaaatacaaacattagctaggcatggtggcacgcacctgtagtcccagctacatgggaggctgaggcaggagaatcacttgaacccaggaggcggagtttgtggtgagccaagatcacgccgcggcactacagcctggtcaacagagcaagactccatctcaaaaaaaagaaaagaaaagaaaagaaaagtagacagcagaaattagagggagatggcaggtgacctggggctgggggaggcacaatttttcttacatagtgatagggctcaagaaattctttctaaaattgatgtatgaggaacgaaattttaaatatagattgtttagaactataagtagcaccaacacaggactccagtaatcacacagcaaacaaaactgagaaaacagacatgggcggggagatgggaatgggtgatttccattccctgctttaatgatgaggtgtcagtagatgctctcttgagttactaaattgagaaacaaagataaagtatattgtttagaggagtgatgttcaaactccagaaaaggctgggcgcggtggctcatacctgtaatcctggcactttgggaggccgaggtgggcagatcacttaagcccaggagtttgagaccagcctaggcaacatgatgaagccctatctctactaaaaaaaaaaaaaatacaaaaaattagcaaagcatggtggtgcacacctgtagtcccagttattcaggatctcactactgtactccagcctgggtaatgagagtgagaccctctctcaaaaaaaaaaaaaaaaaaaaaaaagaagaagaagaagaaaactccagaatatataaaaatgaaaagagggcggaagagtggaactgcggggtagaaaattttgcattttgtttcatctctctgtatatgttgaactttttttttaacctacatgtactacgtcatatccacagcccccaaccatccaccaggcatcaactgctgtgtttatatggagggttgagcaatcattcctgcctccttccagttcgtcttcctgtactgcagaggctagaaaactaaatttatatcacccagattcccttccagctaccttaatgctaccacctcattcagccatcattgattctccacttcaccaaactggtcaataattgtctcttctaaaaatattagaattgggactaagagacactacagctgacccttaaacaacatgggtttgaactgtgctagtctgcttatacacagatttttaaaaaaataaacatattgaaaaaaattttggagagatgtgacaatttgaaaaaactcacaaaccacatagctagaaatatcaaaaaaaaaaaaactgaaacagagttaggtaggtcaggaatgcataaatatatatgttaattggctgtttatattattagtagggcttccagtcaacagaaggctattagtaattaagtatttgagtcaaaagttatacacagatttttgatgatgaggaggattagtgcccccaacccccatgttgttcaagggttagctatatttgaaatctgctggtagctggaattgggcaactaaattcacgagatgtggaacggtcgtatacatggagaataaataaataaataaataaataaataagtatttgttggcctggtgcagtggctcatacctgtaatcccaacactttgggaggccaaggcaggcagatgacttgaggtcaggagttcgagaccagcctggccaacatggtgaaataccgtctctactaaaaatacaaaaatcagctgggcgtggtggtgtgcacctgtaatcccagctgcttgggaggctgagggatgagaattgcttgaacctgggagtcagaggttgcagtgagccgagattgcaccactgcactccagcctgggcgaaagagtgggactctgtctcaacaacaacaacaaaaaagcaattgttaaaagaataagaaaacaagccacagattgggagaatatatttgcaaaacaaatatctgatgacccaaaatacacaaagaactcttaaaacccaacactaagaaatcaaacaaccccattaaaaaatggacaaacgggccaggcatggtggcggtggctcacatctgtaatgccagcactttgggaggccgagcagggcagatcaccttaggtcaggagttctcaactagcctggccaacatagtgaaaccgtctctactgaaaatacaaaaattagccgaccgtggtgacgtgcatctgtggtcccagctacttgggtgtctgcggcaggaaaatagcttgaaccagggaggttgcaatgagctaagatcgcgccactgcactccagcctgggcaacacagtgagactccgtctcaaaaaaaaaaaatgggcaaaagatgtgaacagacacctcatcaaagaagatatacagatggaagataagcatatgaaaatatgcttaacctgatgtcattaaggaattacaaattgaagcaacaaggtaccactaaacccctattaaaatggtcaaaatccagaatgctaaaaacaccaaatgctggtgaggatgtggagtgacaggactctcattcgttgctggtggaaatgtaaaatggtatggccatttttaagacagtttggcagtttcttacaaaactaaacatagtcttaccatacaatctaacaattacactcctagatagttacccaattgaggtgaaaacttatatccagggccgagcacagtggctcacacctgtaatcccatcactttgggaggccaaagaggaaggattgcttgaggccaggagttctttctttttatttatttatttatttattttcttttgagacagagtttcgctctgtcacccaggctggagtgcagtggagtgatctcatctcactgcaatctctgcctcccggcttcaagcgattctcctgtctcggcctctgagtagctgctgggattacaggtgcacgccaccatgcccagctaatttttatatttttagtagagacagggtttcaccatgtcagctagactggtcttgaactcctgacctcaagtgacctgcctgcctcggtctcccaaagtgctgggattacaggtgtgagctactgtgctcagccaaggccaggagttccagaccagtcctggcaacacagtgatactctgtctctacaaaaaaaaatttttttaattagtcacatgtggtggcacacacttgtagtgtcagctactggggaggctgaggctgaggatcacttgagccccaggagtttgaggttgcagtgagccacgattatgcctctgcactccagcgtgggcaacagagtaagagaaagaaagagagagagagagagagagagaggaaggaaggaaggaaggagaaaagaaacgaaaagaaaagaaggaaggaagggaaggaagggagggaaggaagggaggaaggaaggaagaaagaaagaaaggaaagaaataaaaaaggaaaagaaaagagagagagaaaccttatatccatacaaaaccctgcgcaagaatgtttatagctgctttattcataattgccaaaaactggaagcaactaagaagctctccaatgggtgaaaggataaacaaactgtggtatatctgtgcaatggagtatcattaagtaataaaaagaagacttagcaaaccacaaaaagtaatatgcatactaaatatgcatttagtaatattaatatgcattaagcaataaaaaaagacgtgtcaagccacaaaaagtaatatccatattactaaatgaaagaagccagtctgaaaaggctacgtgctgtgtgatttcaactatttgactttctggaaaaggcaaaactacagacagtaaaaagatcaagggttgccgggggttctggggacaggaaaggatgaataggtggagcacgggaattttaggtcagtgatattattctatatgatactataatggtggatccatgatatgcttttgtcaaaacccatagaaagtacaacacaaagagtgattcttaatgtaaactatgtgctttagtcaacaatgtattgatattagttcattaattttaacaatgtaccacactaatccaagatgttaataatggggaaactgcgtgtgagggaggggatacatgggaactctgtactacagctcaataattttataaatctaaaactttttttttaaataaagtctcttagaaaacaataaaaataaaataaaaagtcaatttttttttttttttttgagagggaatcttgctctgtcacccaggctggagtgcagtggcacaatctctgctcactgcaacctctgcctccttagttcaagcgattctcctgcctctacctcccaggttcaatcaattctcccacctcagcctcccaagtagctgggactacaggcatgcgccaccacgcctggctaatgtttgtatttttagtagagatggggtttcgccatgttggccaggctggtcaagaactcctgacctcaggtgatccgcccgcctcagcctttcaaagtgctgggattacagggttgaaccacatgcctggtctaaaatgtccatttttaaaaggcagtctggctggggcagtggctcacgcctgtaatcccagcaccatgggagaaggctgagcagggtgtatctcttgagcccaggagttcgaggctgtagtgtgctatgatggtgccactgcactccagcctgggtgacagagtgagactctgtctctaaaataaataaataaaaatacaataaaattttaaaaggcaatctgcagcaagagaagatgaagttagcaggagagcaggacagaggagagtccatgaacaacagggagaggaaagtggtcatggtggcagctcccaggctcctggcatatgttcaattccctgttcccagccctcaggaagcccagatgtccccacctgcccctacatacaaaccctggatccttgacatcaacttctcctttccttcatgtgctctaatgagtttttgttacttgtggaacatttaactaacatcattaaccaagtggacctgtctcagagtggtgttatgagaagacagcagccaaaagacagctgcagccaagcacagtggctcatgcctgtaatcctagcattttgggaggccgaggtgggtggatcacctgaggtcaggagttcgagaccagcatggccaacatggcgaaaccccctctccactaaaaatataaaaattatccgggtgtggtggcgagcgcctataatcccagctacttgggaggttgaggcaggagaattgcctgagcccagggggcagaggcggcagtgagccgggatcgtgccacttcactccagcctgggtgaaagagcaaaactctgtctcaaaaaaaaaaaaaaaaaagacagctgcaacaaatgtcaagttctgtgtgttttcttttcttttcttttttttctatttaattaatttattttagagtcagagcctccctatgtcacccaggctggagtgcagtggcacagtcacagctcactgtagcctcaacctcctgggctcaggcgatccttccacctcagcctccttcctagctgggactacaggtgtgtgccacgacatctggcttgtgtgttttcttttctttttttttgagacggagtcttgctctgccacccaggctggagtgcagtggcgcgatcttggctcactgcaacctctgcctcctgggctcaagcaattctcctgcctccgcctcctgagtagctgggaatacaggcgcacaccaccatgcccagctaatttttgtatttttagtagagacggggtttcaccatgttggccaggatggtctcgatctcctgaccttgtgatccacctgactcggcatcccaaagtgctgggattacaggcgtgagccactgcaccctgcctggcttgcatgttttctgacatactgtcaaaaggatactcatactaaatggcaacacattctcaagccccttccttttcttctcctatctgctttaccacacaccatagctgcttttaccgttttctccttaaaaactcaaaaaaaccttcccccaacctatctatccacttcttgtcctcccctcaaacccactccattttgattctgcatcataaaaaccaagcagagttctggggaggcagaagcctcctcttatgatattggaagggggtggatgaaattgagcacacaaagccagaatcctcttttgtggaaagatgggggcagtgggcagagggaagcaggctcatctctttccctctcttcccttcccttctctttcacaccacacgctccgcctgggtgagctcatcgtcctcgtgtcttcaatttccacctccaggagatcgactgccaaatttctacccgcaatcccaaactccactctgagctccagacccatctactaattgcctagtcaacattttctctgtgggatcgaatcagataggattatattctgctgtgactaacagagactgaaaattcagagtcctaaacaaaagactttctttttcttgtgctgaaaagtctggaagtacacagccctaggctggaatagggatactgctggggggctccgcctccttgcaggctgcccctctgctatctccagggtgtgtccctcaaccacactgtccaaagtggtgactgaaggaccagtcctcccactgacattccaggcaacagaatggagaagaacaggctgaggaaggaggaaccacaggcgccctccagctgtttcaaggaaggttcctggaagatgctaaataacaattccgtttatatctcattagccaggcttagtcacgtggcaacacctagaagcaagaaaggctgggaaatatactgtttctgctgggttaccgtatgcatgactaaaaagagagcgctctgttcttaggaaaatgagaatagactgggttgggggaggataaccagcattggctacactgcagccccacaacctcctcatgcttgatgtggctgaaataaaaagagcatctccccctaacatttcctttcccttagtcttgttccttttctgtaccccataccctagcagaggggcaattctgtggggtgggctcactggcaaggggaggtctcacccaagctcagtcaagggtgagcagagagaattgtacaaggaagctgaatttccagctggtctggaggaaacactgaaaaccccaaacagaagatgtccaggtggagcaaatgcagctgagagttcttgtccaaggggacagaatgtcagcagagtaggacacaagggcaggtctctactgaaagcaccaggggcaaagtcacccagcccttcaaccggctggccaccaagcacatcggctccactctgcctcctgcccgtgccaccctttgcacttttgtctgtaccatctcctccaccaggagtgcccttcccgctcctccatgtggtatactctcagccacccctcaggctcagatccagagccacctcctccaagaagtctccacctgtctgctgggaaccctcacatcacactgggagacgaagccaaaagtgggggggtcagtacagaggtgtgggaggtaggtccaatcctggctggacctggctttgggtgccatttgggacagggtattggggaggttcagcaggaaggtgggacctcaaggctttctttttatgaaggaggaggaaccagctgttgacagagcagcaacagcagggagccaccaaggctgagccttgaaccctgggccaccaggctcctgcaggcaactgggagcagctcagcctctcacagtcccatttccagggaggaagatgatgcagcctgaggcaattggccacctggaggaagttcagctgggtggggcaggcagaggaattcctggaaggaagctcacccctcagcactggggtagagggacactagagacaggggtggcccaggagctgcccacctcctatccccatgaataagcaggtgggcccgggtgaccagctgggagccacccctgggtgctcaggtaccatgcttctggcccagcctcctgagctgtggcacaggcaagagcaccggcccacggggtctgcatggggcagtgacctgttctctctaagcttcagctttctcatctgtagaaagagggtgaagagtccttccccaccatggttgtggtgagggtaactgaagtgtcagggtaggggacccagtggggcaccagcacacctgaggttcagtcaacaaggcccactggtgaagaaaacctcgcccaccctcccgtccctcctgaggcaggtggtggaggcacatgccctctccattgtgtgtgcatgagtgtgtgggtgcatgcatgtgtgtgcatgcatgtgtgtgtgcatgtgtgtgcatgcatgtgtgtgcgaatgtgtgtgtgtgcgcacatgcatgtgtgctaagctctggagaacaagcagccaggcttgggcaggagtccgggggcaagggggggacatgcaaagttctctccacagcccctcagtgctaacaggctgaggtgggggatgacatgtcttagaaacaaacttcaccctctgccctaaaactgcctgctggtctgccacatcgggccaagctgtgggtacttggaaagagggccctgctcctgccttccaaacccaggaagtcttgaaatccccatggaggggacctggggccaggggactccccaagcagacacagcacccacacaagggtctaagggacccctttctccccaaccgcctgaggtagggatggatgtggacaagcagctcagggctggggctggcctaggtggaaggttcagaagcacagaaggcaggacgtgcccaaggggccggacatggggttggaggctaggggctggaaaaagagtttgggaggtcagccaagggcagtccacgccttggctcttctctgggtcatggggagccacggagagtataggagtggtgatgggatggccccagagttgtgcctttgaaagtcacctctggttgcagatagagatggactgggggcagggctgagaggaagggcatctcatgagaggggaaatccctgaccacagtccagaggaaaacagcgggcctggggagaggctgctcctggggaggacctactcagaggctgattcttcctcgtgctaccccacccagggagatgcaggaagctggtgaggtcagtgggtgggcccacaaccatgctggagaggagcaggagggagacgggagggcacagagaggtagagggcagggactctgaggaccctcatccatgcccacctggacaattgaatcgagcttcctctagtgtggaatgtgggctcactggggctatttgctatccttctctcatgtcagactggatgcggcccaagggtggggtctgtgactccctcatcagactgaggggggccgaaatgtgggggtgggggaaggagggagcttccaaacaggaaggtttggagggcgctggcactgaggacaggggatacgccccccatgagtcttcacccctgctcagagcctgctgccgtggccctcagagacgagcccatctttggagctggagttggggacagggaaaccaggtggaagggaaaaagaaaaaaggggcaggcatgagtgtgaggggccagaccctgcagaccccaggacatgcacagcagacacacactccaatccctcctggctctgctcagcctcaaacgaggtgcaggcctgcaggccccaaccaaggacccctgttacacacagacacaaagacacaagatgcagcaacacacacacgtaatacagaaacacagtcacacactagcgcacacacagacacagtcagacaatccaagaaacacactcacacatgaacagacatataagcaaagtcttacacatacaaagacacacttaagcacagacacaaacacaccaacagatgcacagacggacacacatagacgcacactgggaggcggcctgtggagactgctgattggataccatgttggcttggtatccagaattgttggtgcagcgcactccccatgcctgacctcattaaggaggccctctggggcagtggctctgagaagaaaggcagcaagcctagggaggtgacactgtagctggggagcattttggctgggcagagaggaggaaggagagcatttcagggagggaaaatagcccttgcaaaggtttggagtggaagagcaaaagggatgtcccaagaactgtgcctcatttgatggggctgtggggcccttctctgacgggcagccttcccaggcacaaagggaggtggactgagaagcatctggaacttctgtgggggcacatggggcagaggggaaggggaacaggcagaacgagtcagctgcctttgaaagctccacgagggacctagcagaaattcattcattcatccgttcattcatttatttatgacaggttctctctctgtcacccaagctggagtgcggtggtgtgatcacacttactgcagccttaaactcctgggctcaagcaatcctccaccccagcctcctgagtacctaggactacagacgtgagctactacacctggctaatttttaaaactttttagagacagggtctccctttgttgcccaggctggtctttgttgcccagccagcttcaagccatcctcccacctcagactcccaaagcgctggattacaggcatgagccacgggttcccagcccagaaagagaccttcaccccacaaaccccctgtctggaggccaagtcccagcatccccagaagggccccggttaggacaagaaaaaggctgtgtgcctcggaggagaggctgggtgccccctggtgcggagtggtccccttgccgggcatggggcctcagtctgtgagactccgcgcctccgtcgtctgaacttgagcaaagacagagctaggcctagcctggggacccgagggcctgccctcatccgcaattctgcctcttttctggccactgaggaccgggctagggaagctgtaggggcagtaggggtgggaggagagagggttccgggtcaagggactggaggggtctggggcagggaagtggaggggtctagggcgggcccttccccctccgctgttgggccctcgctgaaggggccgaggctgagccccaaggtgggcctgtgggggcggggcgggggagcccctcagcagctggaccgcaggaggccgaccagggtctggaactcctcttcccctccctccgccgccccctctcccttctcccaccccctgggctgctaggggagcgggggaggcgcaggaggggctcagggaggggccctggacgcgggaccaggctgggcccctcggcggaggcccgcgcaggcaggccctgccgctggcctcgcacatctgggccgggagcgcgggccgacccggcggcgcaggcggcgcggccatccggcctgggggagggggcggcggccggggtgggccggccccaagaaggcggaaggcctcagggccgggcgcacgttgagggctgcgaccgccgcagcgggcacggcccaccgagctgggggcccgggatcgcggcggctggacggggctggagctgtcgggagggcggaggtgagttctggggcgggggctgccgggcgccccgagtggagaaaggcgaggaggtttgccgtcccgggctgtcggctgagaccccaccaaaaacctccagactttgtctggtggggacagatctgcaagcccctctctgcagcgtggctgcgggctcgggaaggcacttggggcagcgaccttggtctcccacctgggcttcgggggaccacctcccctgtccctcgcacagctgctcagaggcttaggctggagaaactcctgtgtcttaggggctggcaggactggtgtgggctggggcctgctccagggagccaaactgagggacccggtgcctaggtctgaggtggaaacctgggagttgagtgtatgtgtgtgtgttggggggtgcctcaggggtcccaggccctgtggtcccgaagcgcaacagagtactgagtggcccaggtgccagggtctgaggctgggacctggtcttccgggccagcttgaggtccaggtggggtggggtccaggttcccgctagggcagtggagcctatctgtgaggttagcgtgtgttgatgagagagactggaagggagtgatcacgaaactagggaccctccggagagcagacgcagcgcaggaatcccaggccagaggggtaatggggggctgtgggcagagcatgggaggggctctgttgtttgtgaactggctgctcccatgatgaggacccaagggggtgcggcaggatgtatggaaagcaaagatgaggacagggaggggccatgggaggccatttctgtcactccctgccccagcgcaggatggggcagccacctcactcagccacacatggtcccatcttcttaagggctcctctccagaacagagtggggcagccctttgggtgccgtttctgatctgtgctttgggccctggttggcccagtctgtggcttggaagaaacgcctgtggcctcacttagcccgctgacggagtcctgttccattacagggctggctgggtgctgagcagagagcagcaggcggaagaggcagggtcctagtgaggaggggagctggaatgacctgggtgggctctttgtctacacaggcatcgtgtggagccctgaaacccagaacactgcattcccatagtgctcctggggacatgagcgcccagctgaaacaccccacacaggaagagatcctccgaggattctgtcaggcaaagggttgggcccctgcaaggtggctcacgcctgtaatcccagcactttgggaggatgaagtgggaggatcgcttgaggccaggtggtcgaggctgcagtgagccgtgatcgtgccactgcactccagcctaggcaacagagcaagaccctgtctcaaaaaaaggagcgggagtgggggcagggttcgtcctaggctattgtaagaccaggggatactctgggatgggagtctctggttgtgagttcctgctgggccacaccctatcctggtccccagtgactaatcggccctgcctattgggtagaagtcagggccaggggtcagggtgaagctgatgactagaggtcaatatgaagccaagagttaggggtcagtccatgggcagggttagggatcaggctggggccagagtttggcatgtgcttgtagccaggaaatggagttcctttctgtgtaaatcaaactgtcccactcaattactctcaaacccataggcaccaccagtctgaaattagtaggcccctgaagcggggctcagcctcccctcattttcagctgtggactctgcccagacctagctttcgggagctgggcttgtcccgtgttctgtggtcagcactctccttaagtgtccctgggtgtgtgtgctcgcttggcagtcagccacctcttggcttacctccacctctcggggaatccatgggatcctccttctttaacacaattttttttttttttttttttggagacagggtctcactctgtcacccaggctggagtgcagtggtgtgatcatgacttactgcagcctcaaccttccaggctcaagcaattctcccacctcagcgtccaaagtggctgggactctaggcatgcactaccacgctgggctaatatttgtatttttcgtagagatggggttttgctgttgcccaggctggtctcgaactcctgagctcaagcaatccacccactttgacctcccaaagtgctagaattacaggcatgagccaccacacccagcccttaacacaaattgatttagttcctacctccttccttaaagaactcatgatgactagaatgcgattctcagtaagaccaaaatatctaacaagaaaattcagaatctgcatcatgaagggaaagaacactgctcaaacctgcccaactccctgcttttatcaataccttgcatatctggacttgttaacataagccctctgtgtcacccattctacaggaaagggcactgaggcacagagatgtggcatgatgcactcacgctaggtgccataataggctcttgccatccctgaaaccccagcataggggcactcagcatatcgcacaatgagagacggtagataactggacaagcttccgatagctgggaagagtataggcccatggtttggatgtggaatccagtgtagagcgaggactcagtctttttttttttttttcttgagatggggtctcactcttctcgcccaggctggagtgcaatggcgcgatcttggctcactgcaacctctgcttcccaggttcaaacgattctcctgcctcagcctcctgagtagctggaattacaggcgtctgccactacgcttggctaatttttgtatttttagtagagacaaggtttcaccatgttggccaggctggtctcgaattcctgacctcaggtgatccacctgcctcagcctctgaaagtgctgggattacaggagtgagccacggcgcccggcccagagtctttttttttctggtggttctaggttgtttctcctccaggtacaatagcctcggtccagaaaatccattcattcattcaacaaagtggttccccagtgcttagcacagagcctggcatgaagaatctcgataagtgtttgctgaagaatcagaacaacatacaaataaggctgccagctcctccctcgagaccttgatccagtagtatgctggttaatggtttccaatcagctctggaagaaggataaaatcctgatttatggtgtttgccaatttctgtcttgtaaatactcccagccatgactgatttgaagctaccaatgtgatgtcattgaatgcagaattgggaagagatagaaagaatcagctcgcatgagctggtgggacccagctcagcatcccactccatcatcctgagctgggctctgggctcctgcccctggctcctaagaaccctcccctccccctctctgtgctttcccttttctcctcaacagccagctgctatatgcatgcccacaaccagcacccatccttcttctgattctgattcctgagcatccaactccaatggtacagaggacagcaagagtgtctggaaatctccatggtgctggccatgcaggagaatcctggacatacaagagtgtgtgggagtccaggtgtctgtccacaatggggctggtttgtgtgaactgtgtgagcaagggtgtgtgcatgtgtctatgggcatgccagagccagctctgtgtctgcctgtgttctatagcaaaactttcagattgggccgctggtgacagagaaggaatgcattggccctgcgtagcctcctcctttgtttagaggagcagccaggcccaccaatgagaacatagtgtcagtggtctgcagactggctgagagacactgttgagcaacacttctcaaacttcaatgtgcacctgaatcacctgggaatcttgttaaaataggccagacgcggtggcttacgcctgtaatcccagcactttgggaggctgagacgggcggatcacctgaggccaggagttcgagaccagcctggccaatatggtgaaaccccgactctactaaaaatacaaaaattagccaggcatttttaggtggcattttaggcaaattaggtggcgcatgcctgtggccccagctacttgggaggctgaggcaggataatcacttgaacccaggagatggagattgcagtgagctgagaactgagatcacgccactgcactccagcctgggggacagagtgagactctgcctcaaaaataaataaataaataaataaataaataaataaataaataaatatgaaatacatgttctgattcagcaggtctgggctggggcctgaggttctgcatttctaacaagcttccaggtgataccaatgctgctgatcctcagcccacactttgagtaacaaagctgttgatggtttgaaatatcctgcctatagcctggccccagaacttcatagacaggatcgagcccctcccatcttagaaaataaatattcttcctcacctcccttcagctttagcgccctccaactactgccctcacatgtattcctgatttcagccaagcttttcagaaattctagctcaatatgattgcgtcactacactccagcctgggcaacagagtgaaactccatcttaagaaaagaagttctagctcatgtcacctcttgctcactcctcacctaagcttacctcccttgggccactaaaacagtgtttccagtctcatctggggaccttttacagggatctagctttacccatctcttggtacaatcttcatgagaccatgaggcatctcagtctcctgggccggtatatattcctctgttcagcctttaaatgtaggttctcctcgtgacagagtgacaccctatctcaaaacaaacaaacaacgaaggccaggcacggtggctcatgcctgtaatcccagcactttgagaggccgaggtgggcggatcacctgaggtcaggagttcgagaacagcctggccaacatggcgaaaccccgtctctactaaaaatacaaaaattagctgggcatggaggcactcacctgtaatcccagctactgaggaggctgaggtaggagaatggcttgaacctgggaggcagaggttgcagtgagccaagatcgtgccactgcattccagcctgggcaacagagtgagaccctgtctcagaaaaaagaaaagacaaattgagtcatgcccccaccccaccctgccacctcaaacctttcaagatttgctgttgcccttcatataaaatcaaatctgaaatagggtgcacaggccctgcctgcagggtaagcccagcccagccctctgtccagacttaccatgtcctcttcccaccccgcattatccaactttgaccttcctagcactcagcaacgcttgtcatttcaaaatcacttgcctgattatctcctccatcagaccgttggtttctagggcagggacagtgtctctctgggtcactatctcccagcacctggcacagagcaggcaccagaaaacatttagaaggatcctgattccaagaaagtaaagccattttttgacacaagctgggaactgtgggactttagatgagatttaaggaattactgttaatttcattagacatgagaatggtgttgcgatgacattgtttaaaacgtctttatctgttggagagatgtattaaagagttcacgggtgaaacaacatgatggctgggatttgcttcaaaggactccagccaaaagtaaacaaataaaataaataagcattcagatgagggaacaaagggacaaataaatcccactctgcttgtctgttcctgttgacagctacagggcctgcaaggatgttggctgtcccggagatgggcctgcaggggctgtacatcggtaagaacccccaccattctgccctgacccatcacccactcacccgagcaggcactgagacccacctacaggttgtgcaatgtacctctcccacagagcttcccaacagctcttctgtgagtgagtgtgtgtgtgtgtgtgcatgtgtgcagggcaggtctgtgtttactggacccccctgtgtgtgcagaaagggtctgtgtgcaccggtctgtgtatgtgagtatgtgtgcacacaggactggtctattcacaggaccctcatgtgtatatgagtgtgggctgagtgcgtgtgtttgtgtgtgccagaagggtctgtgcacacaggagtgtatgcatgtgcgtgtgttttgttcaggctctgtccacaggaccctgtgtgagtgtgtcagtgagtgtgtgtctctgcgggcaggtctctgtgcacgggatctgtgtacgggagtatagtctgcaggtgggcatgcatacagagagttctgcgtgtgcatggagggtctgtgtgcacaggagtgtttgaatgtccatgtgttttgttcaggccatgtgcacaggaccccgtgtgagtgcatcagtgagtgtgtgtctctgcgggcaggtctctgtgcacaggatctgtgtacgggagtatagtctgcaggtgggcacgcatgcagagagttctgcgtgtgcatgtgcgtctggccatagcctggtctgggagcaggcttgggttctccgcaggggtgggaggccggaggcaagagggacactcctcaggaagtccattcacaaagacctggtgggctgggcccttccttcctgcctcccttcccaagctcgagccacagcaacttggcctctcgggctgcagctggtggcagggctggagcctgccctggctgcctgaggagagggagcccttgaaatgtggccactttttccctgcccctccagcacctcagactctggccctgtccaggcgcttcccagagtccccctcatgcctggcccttcgcctgtgttcccaggttacccccaaggccagggagagtggagacctgcctggcagcacgtgacaggactgtggctgagggagcacagggcctgcctggctcagcctaaggcgagccagacctgcctgatttgtccatggagcttcctaccagatctcactctccctccctagccagcctccctggcctgtccatggatatcaaggctgccccagtactcctcacatgccccttagcaatgcccagtgtggtggtggcagctaccagccaggcaggccctcctgccccataagccgcctgatcctcggggcgaggcctggggctgtcctccagggctactctactccctcccaccaaaggtccagctcagtcccaggcgtgcaagagggtcttgatggggcgtcctgccctgaaatgctggctcagggtgtgtgtatgtgtgtgtgtggggggtgggaatcagacttcctaatgttgtggacacccccagttcaggactgggctatctctatgggtgtgggaggaggtgctccccctcctcgatcctcctcaagcagccagctgggcctgtctgtggcccttgtgggcatgggagctgcagagttccctcaagtcctgattagaaccacgtggcccaactttagggtggaggggagcaaccccacctcctagccagttagctctgtagctccaccttttgaaggaggagggcttctctctcctctactccctcccaggagctcacctgccctatctcccctgcccttccatctctggactgagtctccctgccacacaatgcttttctgtctctctcccctcccacactgtgtgtctgaggggccctagtggggagacagtgagctcctggggaaagcatgggtcatctgctagggaaacgttgcctctgacagcagaccctcagctcctgcccctgcgtcatgtgtgtctttcctacaccccgcagtcctcctccccagcagagagccctttgccttagcccagcaaggactgatggagaacctttgggtgcctggcttggggccccgctgccatacctaccctgcttgtgccaggatgaactgccgtctcttctctgcaggctccagcccggagcggtccccagtgcctagcccacccggctccccgaggacccaggaaagctgcggcattgcccccctcacaccctcgcagtctccagtaagcccagagcagggaccaggtggtgggtgacctggctggtgtggacagggtcgtgcgtggcaaagtcatgacagggcctcagctaggaaggaggaggggatgggggtagcaccatgcctcttgtccccgtacactccagtcatgtgccccccagaaggattggccttgggtgaccctggactataaagggtaacaattctactgagaaggcgagccccatgtaactaactcccaacccccccacacaggaggcacccacggccattgaagctggcctggctgaggcaagtgttcccctgagcatgtggcttttctaggtccccagagcactgggcctcttgtggaccacagcccaccttaatccaagtcatatctgaaggagaggagatgtgttgccaaaaaaaaaaaaaaatagtctggaaaccattagcttgggctgggcatggtggctcatacctgtaatcccagcactttgggaggccgaggtgggcagattgctttgagctcaggagttcaagaacagcctgagcaacacagcgaaaccccatctctacaaaaaatacaacaattagctgggcgtggtggtgcacacctgtaatcccagctactcgggaggctgagggtggagaattgcttgaacctgggaagcggaggttgcagttagccaagatcgcaccactgcatttcagcctaggtgacagagtgagaccctgtctcaaataaaaaggaaactactagcttgaagtatccaggacaccacctccccacccccaagctccctgggtcaccccacctggatgaccttgccctggtgatccagcttagggacccccttattcttaggggagtccagccctggcataggagaaggcacacacttctctggggacatgcacgtgccctctcctctctgccacagaatcagacagtctggtttgtgtcactatggccacaaagagcaagaggataaaatatatacactggtccatgtgctgtaaaactgcctggaggagcaaaattgctcccagctgggcccagactaatggctgcctatgaagatggtgtgaggtgggggtggggctaaggcagtccagagggagggtgggcagaggctggaagcaggaaagcaggagcttgagccaaaccctgcctggggctacctgagagacacgtccaaggctcagcctggagcctgggagggcaagggagcccgagcaggtgggcaggtggggtgggccaggtccagggctggtctggaccacagtcagtggaggggagtgtcttccccatgcagaggaagcattggggctgtgggggatgggggtgtccctgctggctgcttgctaattctaagtcttgcacttgcagaaacccgaggtccgagccccccagcaggcctccttctctgtggtggtggccattgacttcggcaccacgtctagtggctatgctttcagctttgccagtgaccctgaggccatccacatgatgaggtgaggtcggctgggctgagagagtgaggtggggagggtggggagttcctcataccttggtcccaaaagtactgtcaccgagacatggggtctccttggaggccctgccacccccagtctggggctccccactggggtaaaagttggaggatgggccccaggcctgggtccttggcctcaactggagcagctcccaaccctctgtaggaaaagtcagactttgtttttaattttgaatttttttgagatataacaaacataacaataaaatatgcaaatgttttttattttcctttttttttttttgaggcggagtctcactctgtcgcctaggctggagtgcaatggcacgatcttagctcattgcaacctccacctctcgggttcaagcgattctcctgcctcagcctcccaagtagctgggaatacaggtgtgcgccactatgcctggctaacttttgtggggttttttgtttttttttgagacggagtctcactctgttgtccaggctggagtgcagtgacgcaatctcagctcactgcaagctctgcctcctgggttcatgccattctcctgcctcagcctcttgagtagctgggactacaggcgaccgccaccacacccagctaatttttttttttttttgtatttttagtagagacggggtttcaccgtgttagccaggctggtctcaaattcctgacttcgggtgatccgccagcctcggcctcccagagtgctaggattacagatgtgagccaccacacctggcctttttttttttttctttgagacagggtctcgctctgtcacacaagctggagtgcagtggtgtgatcatagctcactgcagcctcaaactcctgggctcaagcaatcctcccacttcagcctcctgagtagctaggactacagacagcaccaccacacctggctaatttaaaaaaaaaaaaaatttttttttggagagacagggtctcactgtgttgccagggctggtcttgaattcctggcctcaagtgattatcccactttagcatcccaaaaatgctggaattgcaggcgtgagccaccatgcccagcccacaaatatttttctaagcttgtagacaaacacacacatatacaaacatatatgtgtttttttaagtaaaagtgagatatacaacttgctttgttttaaagcttcaacactattttgtagatatttcatgtcagcacatacgaagctaccactttctttgaacggcctagtattccatagtacagatgtgtcataatttatccattcccctattgacagttttaggggggttttttcctcctctcatttttcactattacaaaaagtgcagcagtaatatccttctttgaaaacttgtgccgcaatctccatggggtagattcctagaggtgattcctaggtccaacatctcaaatcttttttcttttccttttttagtaaaaaggaaaagaaaaaataataaaacaagaaataaaaataaaaacaagaaaatgaaggttctaagggctgagttgacaagtcacacagttgttttgaaagacatgtttcaaaaatccattcaaatgttgggttttatgtgggacctcaaggaccttgggacctcaagaccccagtcctctgaatatgtcctagagggtcggcaggcagtaggtgaagctacaggagacctcaaagctctcaacccagactgggatgggaggtaagaggggcttcccaaggcctggagactggatggaagggtgaatggagacatagacagacaggtaactgctcccaaaatacatgggacttgagcttcatgagactggactgtccttccttccacatcacaggaaaccacaaaggctctgagcacttacagccctctaggagctgcagggcatcccagatgtcttgctgatcaacacacatggctgaggccatgctggggctaaggacacatccaggaacactgtctctgtcctgtggaggtgacagtcacaggaaataactgggggacattccagcctcctcccaagctctctcttcccaccagacaccaagcccctaacatctctagaatacccatctacttctctccatggccactgatcgtgcccaagttcagccccgaccttctctcacctggatggctgcaatatcctcctccttggtttcctttctccatgctcccttccctgcattctgttgctccatgggagccagtgacctttctttctaaaggcaaatgtggtcatacctcttggctactttaaatccacgaagacagaattctgaatggccttctgtgctcttcatgatctcaccttagcattcttgtgttttctcctgctgcctatggagccgacccatagctagttctccctgctctttcacatctgtaagtttttgcacatgctgttccctctgcctggattcccacccaagaagggagggttgcactgactgccctgtgcctccgcaggaaatgggagggcggagacccgggcgtggcccaccagaagaccccgacctgcctgctgctgactccggagggcgccttccacagctttggctacaccgcccgcgattactaccatgacctggaccccgaagaggcgcgggactggctctacttcgagaagttcaagatgaagatccacagcgccacggtgagtcacagggctccagacagggaggcggggccagcatggaaaagggcagggctaatgggggtgggtgggacaaaaccaaaacgtgtgaggaccggcccgatggagtcgtggctgagagggggcggggctaaagggagacgtcggactccggtgtgggcggagctcagaaatgaggtggaggcggggctaatgtgggtggggctaatagtgaagctggggttgcaggaggggtggggctaaggagaggggtcggggcagagctaatgtcacatggggcaagagtgggacggtggtaaagaggaggggaagctccaggaaacggggtgattttaagagcgaggtcgtcaggaaatgagtgccaagctgaggcctcctgcagagccgccctgtgtccctgccaggatctcaccttgaagacccagctagaggcagtaaatggaaagacgatgcccgccctggaggtgttcgcccatgccctgcgcttcttcagggagcacgcccttcaggtgcgctgcggccccacctctgccgactgtggcagggaccccctattttcccctcatccgaaaccgctcccccatcccgtccccgacattggatgggtagccaccgccggagctcagaggtcatcttctccagtaccctcctccctttttgtctggtagagcctgcaccaagccatactgatgggaggggggccgattcttccagctctgctgggaagtccttcctgtgatttgattagtacctccagttccgcagagggctgaagaccaccctccctccaagccagctttcctctcactgccccctcctgtaccaggagctgagggagcagagcccatcgctgccagagaaggacactgtgcgctgggtgttgacggtgcctgccatctggaaacagccagccaagcagttcatgcgggaggctgcctacctggtgaggacgtgcaggcgggcccgagaacactgctcaggaagggccaggcctgtccccatgcttgcatgcaccccaccacccttgagaccacagagtcattgtggaaagaacttcagcctgctcctgatgggagtttgtagagttgctcccaccagaagagggagtgggcctgcaggaacaggggacagagggacaaaagacacagcccaggccagtgtagacagtgccttaagtcaggtgtcctaaaagcagagcccaagatggagagtcttttttttttttttttttttttttgagacagtcttgctctgtcgcccaggctggagcgcagtggcgtgatctcggctcactgcaagctctgcctcccaggttcacgccattctcctgcctcagcctcccgagtagctgggactacaggtgcccgccaccacgcccggctaattttttgtatttttagtagagacggggtttcatcgtgttagccaggatggtttcaatctcctgaccttgtgatccgcccgcctcggcctcccaaagtgctgggattacaggcgtgagccaccgcgcccggccaagatggagagtcttatatgtgaggtctattgaagaaggtttctcaggagaaaggcgagggagggaggcgggacaggagaaggagctatgaaggatgtggtctttgctggagtctagcctcaggtttagcctcagctagatcccatggggagctgcagagggagaactgtagcacagagttggggccggcttcttgcacatccgtatcggtctgtcactggttctggggaaatgggagggaaacctctccaagtgaggccattcccattcagctgagggcagttatccagaggaggtagcagctgtgagccaatagcagccaacactcacagtggcaggcagggcacccagaacattcacttcacttggcatcagtattttgagggacacccccaccaccgcccacctttgttggttcttgagcaggggaaataggactatgaaataaagggaggcgattctagccaatgcatacaggtcttcctaccactgagatgctcaaaggtctagagcaaggagttggggggggctcccgtggagacagagagggcatagcctcaagactcattgcttggtcgtaaccctagagccttggatacagaccctaagctgtccaaaagggatctcaagcctgcctgtggacaccctggagggacccccaatcctcccatacctatgggccaggcatgacccagggccaggtctgtaaatgtctacacttttcactggaaggagctaaccctgagcacagtggacatccctgtgcaaagatcaggatggaagcaaggctgtccccagccagaacaaacacctgctcccccagcgtccccttgcctattttcccaccctccttctcacgctgttccctggctgctcaggaaagtctggccctgggtaaaagtctccattcacacccttatccctctcccctatctggtcagaatctggggaaccctacaaaaccataatatacttcccacagggatcccctaccctgaaggaaataactccagatctcaagtgtttttctccacccaactggcctgtcttggtgcctggaatttcagcttcctccctgctgtatcagattccctgggaacaaagttctcctggaagtggggtggcctatggctaccgtctgacaccagcctagtggagaaagagggttcaatgagctaaaagggctccccaccacctcctctgagccatcacacacccccacattgtgctagagtctctgccaatacccccagccagcgctcagctggccgcttgtacctgtgaaggggaaccttgctgcagccccgctatcttgggaaatttgaaggggcagatcccaggggttctaggtctgcattctgtctagagtcttttcctgctgggcaacttcttggcatatttggccagggccattccctccccagcctggtcacagcgtggctatgaggcagctccaaatttgtgcagcacagaggggcctgagaggcctaacatgggtggggtgtgatggaggaggaggtgggagccccacaggccgcggttccccacctcacagtgccatcttaggtgtgacagcccacagtgctgcctgaccctgcccaccacccatccccaggctggactagtgtcccgagagaatgcagagcagctactcatcgccctggagcccgaggccgcctcggtatactgccgcaagctgcgcctgcaccagctcctggacctgagtggccgggccccaggtggtgggcgcctgggtgagcgccgctccatcgactccagcttccgtcagggtgagctgcccccggggacaccacccacccctggagggtcagagggtcactgaagccagaagctcagccatgtctagtatgaaggggagagggtacccaccctggaggagcccaatttgagcaggcagaaacatggctggtggagcctgtctgaggaagggaggcacagccctgcccaagggcaacctcgtctcagggtgggacatgccctacctagggcagcccaatctcagggtggagatttcaccttgccctaggggagcaaagtctgaggagggcaggactccgcccagccctgagtctgagggttgggggagacacagactaccccaggaggaaactgctggggtactgacaaaggaggaagtcagcccactgcatcagctgtccccttcactcccctcatcttccccgacacacatcagccccaagctcctgctgccaggaccaggcacccaggccaaggacagctatggtcatcctcccaaatgccatctcccagggcagggagaagccctaagtcctgagtcccctctgagactccaaagacctacctgcctccgctcctccaaacccctctaaccctgattttgccatgacctgagacctcctgcgttaaaggaaggccctgtgtccataaatatttccccacagctgttggatacagggtgggagtttggggttcaggattgccctctcccagtcaggagcaggttggagtttcaggagcactggctgctcccagtgcccatggaggtcctgggcaggaggatgggagttgaacgccatagctggagcacctccttctaatctcactccctgctgtctcctgacccccagctcgggagcagctgcgaaggtcccgccacagccgcacgttcctggtggagtcaggcgtaggagagctgtgggcagagatgcaagcaggtagggggaaagggggacggagtgttatccttggcccctaccgggcaccatatactgatggggggaagggcatgtttgcaaagcccgtctcttcctcctccattcgctgtacccaacctggccgtcccctcacagtcacccgcacccccaccccactcacagcggcgcccctaactcccactcctccaggggattctccgcggacgctcgggtggagttgcagagcctctggaaccatttctcccccacaccctgcgcccatatgtggtggtctgaggttcaagcacctgaagcccctcacgtccctcccccgaccctgcagacaggccttgggacccggggcagggctggaggctgggcgaggctggagggggcgcagggctgagggtgcgaggccgcccacgagtgtgtgcccgcgctcgccgccgcaggagaccgctacgtggtggccgactgcggcggaggcaccgtggacctgacggtgcaccagctggagcagccccatggcaccctcaaggagctctacaaggcatctggtgagtagccaggcggcgccccggtacccagcgcgacccgggctccggccccgccactgccccctggcggcccggcgagcgctgacgccctcttcgccccctgctccaccccagggggcccttatggcgcggtgggcgtggacctggccttcgagcagctgctgtgccgcatcttcggcgaggacttcatcgccaccttcaaaaggcaacggccggcagcctgggtagatctgaccatcgccttcgaggctcgcaagcgcactgctggcccacaccgtgcaggggcgctcaacatctcgctgcccttctccttcattgacttctaccgcaagcagcggggccacaacgtggagaccgctctgcgcaggagcaggtgggtcctgagcccgcgggctcaggcagggtttgccgacccgggaatgaccgtgcactggagggtcccgggccccaaggaacggtgggggtctgcctgattcatcccacatatacactaagccagcagggcgtcggggtggggcggcggggagcggcgagtgagtgccccagcccagcaggctccacccacggaatccgcagcccgaactggggcaagacagagaatcatagcggggaggcggcaatgcctatctcctcccagccttctctacacccccaccccgggccctgcgggcccatgctcctcggtttccctgcaccaaagcaaggggaggcccctcccaggacctcgtacctggaacctggagcaggctggcaactaaatcctctgagtgagtagggtggagataagggactaacatcccgcaggtccagtcctccagacaccacgtgcagtcggtgcccaggcacttctgcctggaggcagaggtagagaataaggaccacggaccccaaactggggcaagcagctgggccctgaccgatggatatttgcccctttcaccaccaacagcgtgaacttcgtgaagtggtcctcacaggggatgctccgaatgtcttgtgaagccatgaacgagctctttcagcccaccgtcagcgggatcatccagcacataggtgagcacctgagcttggtcccccacccgcccctacatgaacaaacagatgcagaataattcccccctatcagtgcctagatacctccacacatccatacactgtgatgagacctagaatcatctagaacacctgcgggatgaagtgcagtggtgattaagagctagagggttgatatgtagtcttgccaaggcaaaaaacttctggtgcctcagtttccccatttataaaatggggtgatagtattgggttctcaaaacgttatcacagggataaaatgagctgaagtacctagagtgagcacaatgtcttgcacacaatgtctaggtgtttaatacgtgtaaaatgcatatccttatctctcgtcctccacgtcgtggtgggagagaagtggggagcgtgagtgttggggaggcgaagccctcgaggactcccgtgagctctcaaagaaagtgctcaaatggctactttctagtcgccaggtaggtacaggctagggaggggaggcgccggtggccgcctagtggtggcctcagtggctctctctcccccgccccttctcctctgcccccttcacccgcgtccccccgtcctgtcccgcagaggccctgctggcacggccggaggtgcagggtgtgaagctgctgttcctagtgggcggcttcgccgagtcagcggtgctgcagcacgcggtgcaggcggcgctgggcgcccgcggtctgcgtgtcgtggtcccgcacgacgtgggcctcaccatcctcaaaggcgcggtgctgttcggccaggcgccgggcgtggtgcgggtccgccgctcgccgctcacctatggcgtgggcgtgctcaaccgctttgtgcctgggcgccacccgcccgaaaagctgctggttcgcgacggccgccgctggtgcaccgacgtcttcgagcgcttcgtggccgccgagcagtcggtggccctgggcgaggaggtgcggcgcagctactgcccggcgcgtcccggccagcggcgcgtactcatcaacctgtactgctgcgcggcagaggatgcgcgcttcatcaccgaccccggcgtgcgcaaatgtggcgcgctcagcctcgagcttgagcccgccgactgcggccaggacaccgccggcgcgcctcccggccgccgcgagatccgcgccgccatgcagtttggcgacaccgaaattaaggtcaccgccgtcgacgtcagcaccaatcgctccgtgcgcgcgtccatcgactttctttccaactgagggcgcgccggcgcggtgccagcgccgtctgcccggccccgccctctttcggttcaggggcctgcggagcgggttggggcgggggaaacgatagttctgcagtctgcgcctttccacgccctccagccccgggggagataaggtcatgggagagtgggtggggacacacccagagactggctttgggattgggcactggtccgctgactgccaggctgaagggacccgccaaggactgaacgggtaagagaagaggtttgcaagacagagcgcgcagcccggcaaggggcatgtgaccccgaaggaagaacgcaacagaagagtcctggtctgaacttggccgagtaggggtgggggtgggatggcaggaggagccgcaggaggaaggaggttgtgcagggtctggacctgcagggctgaagttcactcatcgaccgactcagccccaaccgggagccaggcagaaaaaccctgtgccgtaggaaagtgactggaagtggactccagagggacaggtgtggtggcacagtcctggtgtggtgctgaccacccaaatatgactgtgaattgtggaaagggcagtagatctctaatgtggaggtgggaacattattgtggtggaggcaattatgagggtagcatttctttcgagacaaaacacccgtctgggaaggccccaaggtcagcttatgaaggaccccacttgcaccccaccccagccatggaagagcagctggagggtggatggggaggccagagggagcaatgaggggtggtcccagctctgctattgactcggtatgcctttaggacattctcttaccgctcatgggcctcagtttcctaaagtgtgaaatgtcaggcacttccctctaactggcatgcaacagccccacctgcctgagagccctgaggtgacaataaaacatttatgctcaaggggaagccacagcctgctgatatggcgtggagaccctaatagtgggaggaatgcaagggttcccggtgctagagagagaagggagaaagctttcagctgtgcatagggaactgaccagaagggggtgctgctgtctcccatcaagcatcccaaacaactccactgcttaagacctctctggcctacacatgaggtccctctctcctcattcaaattaattgtcttggaagccagcttctggcctaaaatgccaccacctgtgcatacctcttgtggggctaggtgctataataccacgcggtgcccctgcctcctgagtgagtctacccaagtctttccctggcccatctgcaaaggagtaggcattaccccaaccccagagaacaaaaatccacctggcctccggtatccactggaagtttatttctttagggttctatcccaaccagtcgcttaaaaaccaagtaacacagacctgaggggtgggggctggggactgcacctccctcctactcatggtggacagcagtggggactagggaggggcaggagaggtggctgaagcaaggcagcagtaatggggccacgacgccacagagccagctccgtcctctcccagaccctggtgggagtccctgtggcttggggtggggagtgggggacccaccccaggccctccctctcccttcctcagacagcctcctttcgggctcaacccatttcttccggcaggagactgaggcacacagagaggaggaagtgggagaggaggacgagggaggggcagggtggcagcacaaatgaaggcagaggtgagaggcgtgggcaaggccactccacccccacacccaccccagagaggggcgaggaagccacaccatcacgcagcatgtcggggggacaaggcggggtttaaggctgaggggcccggggcaggcggggcctcgggcctcagtcaaagccgtgccagtcgctgtgctctgagtcgtattccagctcggcgcccacacacttgacaccatccagcagcatgggcgtgccgtggtgccggtctgcaaggcagggtgcaagtcagtgccatgctggcccccggccccacccatgcggcccactaaggggacccctccccttccctcagggatcagctggaggtagggacctgccaaggaggttgagaacccctgagccgggcaaggatcccttgttcagccttggttccctgaggaggacagaaaccctcgcagtcgagcttgtgcatccctcctccaaccaggagcctcacgctctcacccatgacgcgggcctgcaccgtcacgcgcacacaggtgccagtgccaccttcgcaggctagcagtatctcctctttgaggacgccctctttgtgcgccgagtactcacacttgacactataacctgcccggggacaggggcaattggtcagcacctcccccagcctccccgatcctgcccctggcactcacgggcccctacccaccaccatggggggactgcatgctaagcccccccaagaaaggggcagggatggcccttctggagcccagagacccattccccctagcaggggtgcacaggtctcaaaaacccattcttcagtgagctggacatgcctccagcctgtgggccaccccaatgtggctccaagagtgaatgaagtagggtccaattcaggcttccaaaagaaggtctggctgttttctcccaaaaggaaggcagggagaggcggtgatgaggagtgaggggggcagggcagggtaggctttgagcagatccgatggcaagaggtaaaggcctgaggggtgtcaatccattgaagggcaaggtcatgaagaggctggtgacggggacatgaaactgaagctggagctccacgaaactgacaccctagcctctgccaggctgggagtggggcatggggcagggcctgaagtgagcctgataggaacagcagcctgaaacccaaggtctgggactggtgggtgctaggagttatccacacggtgtgtgtagctccagcaggtttttctagagggttagggaggcaggagggaaggctggaggcttcaaaccagttcctcagcagctcccatcttggttactgccccacggaggtaaccatcacaccatgggcaggtacagggaagtatgagctcatggacttctgttcaggacagggagcaaggcctggagtgtgggacctgccatgctgccacagtgcaagctcacaaagaggtgccacatccccgacttgctgagctgcccatccaccctagttggaagtaagggagcaccccatgttctcactcccacacccatccaggccctgctggaggaggggacgcaccttcagggacgggcaccacgctgaggagcttgaggtgcaggctggggacaggtgcctcgcggacatccttgctcagcctgtgcactgggggcagagtgaaggtaatctcatacctgtgcaggatcttcaggaagccaacctacagcaggagaggtgagggagggaggcaccttcacttcctgcttcccacaggcccacccagttgtgccacctatgatcccatgcgccccagcacctgcttccccagagagcccagaaagcccaactccctccatcactccagctgatcgacccccagccctttcacagaccccttgagggtcccagccctacagcttggccagcaagatcctcagcatctgtctcacgggccccaaagtcctcagtggctcatttcatagacaggaaaatggaaaccacaaaatgactggccaaaggctatgtagtgtggccagggtcaagtcctccctagacttcttggccattcctgctatgcatggcacccagcaaggcaccacccactgcctacaagggaaagttcaaagtcccatgggtcattcacctgtccccatcactgccagtatcccagggtcagggatgctggtcctccattcgctcataggatgacaccaggccacagatagcgtgggatgagggatgggatcaaattagagatatgacagtagaccagctgtgtcagcccagagctggcccacagacctggcaccagccactgaactggggctcctggctgccctgctggcactggactggggatgcctgcctgcccactcaaaggctgtgtccaatggcccatactcacgacccaagcaatttctgttgttcattcacagtgcttggtgacaaccaccctctgggacaaatgccattcttggaaactccagtagtatgaagggtcacaagccagggtggttgctgagcaggggggctggtgggggtgccacgccagcaggcaaagggtgcctgctgtactcccagcttgcatgggcacacagagtcctgctcacagttactggggctgggcagccccatccctgggggccaactgggactggctgcagagagttttagccattcaatgggaccaggttgatattgctacatgacaaacttcaatccatgtgcccccctcatgctctgctggtagggggcacatccctcaagaaggctttctggctgtacctgctaactgtgaccctgtgacccaggggtactacttatagaattttccaggggaaatagagatgtgcagaaaaatactcttcgctgccctatttataacaatgaaaacaaaaacagccgtaacacccaggaacagggagccagctaagtcaatgatgacacatatcatggaccagtaggcagttgtaaaatctcgtggaaaattatttactgacacaggtaggagacagttcacaacagaagcaagcaaaaaccagtgtggacacggtgaggccagcttttgtttttggaaacaaagctttttaaaataagctggatccatcaccatagtgttttcagaaaaacaaataaataaataaataacattttaaaaagctggaataagccaggtgtggtggctcacgcctgtaatcccagcactttgggaggccgacacggacggatcacgaggtcaggagatcgagaccatcctggccaacagagtgaaaccccgtctctactaaaaatataaaaaattagctgagagtggtggcacgcgcctgtaatcccagctactctggaggctgaggcaggagaattgcttgaacctgggagctggaggttgcagtgagctgagatggcgccactgtactccagcctggtgacagcaagactccgtctcaaaaagaaaaaaaaaagctgaaataacacacatcagaatattaaccatggttattttgggatagtatgactgaccgtacgctgaagaggatgcgtattgactgtgctttccttctttgctggtggatatgaaaactggaatggacattgctatgacgggatggagctttcttacaaagctgagcatgagtcttaccatacaatctagcaatttcactcctagatgactacccaagtgaagtgaaaacttatatccagggccgagcacagtggctcacgcctgtaatcccagcactttgggaggccgacacggacggatcacgaggtcaggagatcgagaccatcctggccaacagagtgaaaccccgtctctactaaaaatataaaaaattagctgagagtggtggcacgcgcctgtaatcccagctactctggaggctgaggcaggagaattgcttgaacctgggagctggaggttgcagtgagctgagatggcgccactgtactccagcctggtgacagcaagactccgtctcaaaaagaaaaaaaaaagctgaaataacacacatcagaatattaaccatggttattttgggatagtatgactgtgggtacttttaaatttcttcatattttctctgtcttccaaattttctcatgtctattaagacatgagtggattttgcaatgaggggagagagctatttttaagtgttggtttgttccatttactctcttggggaagctgtcagctgtaggacaaagagtgggaacttcagagttggacagaaatggatacaaacctgggccccccaggttcttttttgtttgtttgtttgagacagagtctttctgttgcccaggctggagtgcagtggcacaatctcagctcactgcaacctcagcctcctgggttcaagcaattcttctgcctcagcctcccaagtagctgggactacaggcgtgcgccaccacgctcagctaacttttgtatttttagtagagacgggtttcgacccattggccaggctaaacctgggccccttttgagcagcgcagcagaccccccactcagggccatctcaatgggccagacctcccccgacccaagggcactcctgttcaactctggacccctgatttattgaccaacctgaaggcctcagtctccattttccccaatccagctccaccaagcagaaaacagggggtgataatactacttcaaagagcctaactgagccagagatttggcaaagaagggttaaaaaaaaagttgcaccttgttattgccatagttagcttcacacctgtatcacatacatacattcttccattctcacaccaactctaggggatgtgattgtctccacttgagagaaaagaaactcaggtgaccttcccaaggtcacagagccagaatggctggcctagacctgaactcaggccactggcaccacagccacgacactgccttccattgctattgtctgggtgaagcagatgacagcagtggctctgctcatcttgatctggatgacattctaaatgctctcatctcattgaaatctcactgcctcccatctgacagatggggcaaccaaggcacagggaaaggcacagacatgcctataagcccggctgagaggaacggggataggcacccagaagccaggcagagccggggaggggaagaagctgctgatggggtaggtgtgcatgtaccttgaccagaaagctgctgtcactctcctgggtgaccatgaccaccgagtcatgcagcttctcatcaaagtggacgtggctgtgggatccttctgcatcgtggcctgccgcaaagcggatactccggactctgggcttgttgcctaggaagagtgggagagggctctgagggctttccctgtccccaggaaactcctcccccttgtcccctcgtcacccccaagactgcccttgacatcatccagctcccactggctgggctcttttcagggctagatggacactaggatcatgaggcttgaggcctccccgccggccccgacctgcccctcccacaaaaatccatcccctgagagtgagcgtggggggactcagggactggctaatatgacccttgcttggagggggtggggctggtgccagagccaggaagggacagtcttccagactcccaccaagccagggcagcgtgggactcagcccagtcctctggataccctgcccagtgctctccttcacagtgcaaagctccccatccctggggcctacccctaagctgtgtcagtgcatgaggcccctgcctgcccatcctcatgacccaaacctgaaagggcagggacaggaacaggctctgggggatctgcctggggtggcagaaacaggggggatcaaaaaacacactcaggccacctgggcgaggctgcagctgccactaaacctcactggcccctggcagcaagaagagagaatccaaggaagcttccagacccacctcaagacccccttccttcctctgtctagcccaccatcaaggccctcagctgtctctgagctgggtcgggttctccaaggccagaaagggcagacggatgagacaggcagggtaggcgggagccgagctgaaggcctggactggatgaatgctcaggctgtagaggccgagagcacggccctagtcctctctgacccctggcccctaggccctcccaccaagagccctgcccagggtcctcttggcgggaggacctgatccagctttgagccggcctcttggatactcggaggcccaggggaccagcctggcacatccacaatccctggtgagccccacagaaccctcaaatctcaccagatcaccctggcctaaaaccctcccttatcccttctggactctgaaagaaacccaaactcccttccacagcctgctgtggccctcccacatcaccagtctcagtgctcaccataagcccctcggctcccccaacccccacagtggaacggcagggacacaagagcacacacctccacagaccagcctaaggagtcagcagggctctgcaggggtcctgctagggcttgcgtgagccccagaacctggcacaaacactgacatagtcccaatcacattcgcacaccagcacacacgcactcacacatgcacacacacacacaggtggtgcgggccctacctgcagcaatggccctcctaccagggcccaccggaagagcttgtgcaagtcctaccacgccaggaaggcacttacccttgttggctgcagccatggacgccctccctgccacgcagctcctgccagacaccgccactcacgctcagcagcctcccatgctccagggacaccagcgggagcctgaaggatagggagtggggagggcaggggtcagggccacccatggacccatggctcagggacaccagaggagctccctttaggaaggactcaaacccctaccgctctcagcagcccaagtggtgcctgtactctctagcacgggggcttctgcccaccctctaccgactgcccacattcaccaagagccctcaccccttcctgataccccagtgactcagggtcctgccagcatgtgctgagctgcagcttctaggtgccaaagcaacagcataggactcctatccccagcacccccagagactggcaggaggggcagcctggaaaggaggactttattggtattttccagggaccagttctggtgctgctgaccccaaaggctgggcctggagctaccttattcaggtcacacaagcaatgcagctgggtgcggtcagaggcaacagggtgctcgatggtgggcaagaacaggggaccacataaagtaagcgtgcccttagagctttccctcctggtgatcggtcagggccatatgcaaaacagatgcctgtggagggggtgtgcccccacctgaaccccattcagaccctgccctgagtctcaggcctgcctccgcctcagctccccaacggcagcctcagcacaggggcagtgagaggcaccccaggaagcctccaatgggccgagctgggaccatctgagcatcaaaaagaataatgagtgcaactgatggaaacagatggaacacataaaagcagtgcattcacagagattattagaaaagaagagagaaagcaaaacaaacaaacccaagccctcaaaagccctcatttgtcaccactgacggtagcagtgccctctttactctgaaagctgaggattaaagaggaagaattcagcctgtctcttggcctttcttgtaaccaaattgccctggtggttgatggaaagctcttctttcgggaagaattcttgctaataaataacaaagaaatgaccaaatgctaagtcattctgcaacccctaaaggaacaaatgctggaggcaacaagaaccagtggatgctaaaccagaggggaaggttgacaggaagcagatactcacaggcgcccaagcgtcactgacagaggacctggtatggtttttgttttttttttttttctgagacggagtttcactcttgtcacccaggctgggatgcaatggcacgatctcggctaactgcaacatctgcctcctgggttcgagcgactctcccgcctcagcatcccgagtagctgggactacaggcgcccgccaccacgcctagctaatttttgtatttttagtagagacagggtttcaccatgttggccaggctggtctcgaactcctgacctcaagcaatctgctcacttcggcctcccaaagtgttgggattacaggtgtgagccaccatgcctggccgaggacctggtgtttaagaggaatggcacaagtttgcaatggaggtatgtgacttctcccagtcacacactggtctatctctctactctctgcaggacagcctgtcacactgatgcctcctgaagtaaggcagcccaaagtcacagcatcactgacaagggattcctgccaaaaaggtttaacctggaatctaaaccagcctctaacttcaacttctcatttgctaaaaacacagaggagaggggaacaaatttaatgactccatgagaaagcaatgagacgagcccagaaggcaggatattctacaggatgactgacctgttttttgtttttgtttttgttttcaaatgtcaagaagaaaaagaaagcaggctgggaacggtggctcacgcctgtaatcccagcactttcacggatcacaaggtaaggagttcaagaccagcctggccaacatagtgaaacccagtctctaccaaaagtacaaagattagccgggtgtggtggcgggcagctgtagtcccagctagttgggaggttgaggcaggagaatcacttgaatccagaaggcggaggttgcagtgagccaagatcgcgccacggcacttcagcctgggtgacacagcgagactctgtctcaagaaaaaaaaaaaagaaagaagaaaaataaaacgagggagattattctagataggaaagcagcaaactacagctgattacacagctgatggtaaggttgctgcctgttgtgtttttgtttttgtttttgagacagggtctcactctgtccagcccaggctggagtccagctcactgccaccgtaatctcccaggctcaagcaatcctcccaccccagcctgccaagtagctgagaccacagccacgcaccaccatgcccggctaatttttgtagagacagagttgcccaggctggtctcaaactccggagcttaagcgatccacccacctcagcctcccaaagtgctgggattacagacgtgagccgccaggcctggcaaataaatttttgcaaataaagttttactaatagaacactacacttcattcacttacatgttgtctgtttttgcactaaaacaaagttacttccaagtagttatgatagagaccatatggcctgcaaagtctaaaccatttattatttggcccttcagagcaaaagtttgccaacccttgttctagatcaacacacacttaacagaccaaaaaaggataatcttcaggacaactgacctgtttttttcaccacatcagctgcaagaagaaaaataaaggagagggtgtttcgaactacctatcaaagaagacattttggggggtgccgggcgctgcggctcacacctgtaatcccagcactttgggaagccaaggcgggtagatcacctgaggtcaggagtttgggaccagcctggccaacatggcgaaaccccatctctactaaaaatacaaaaatcagctaggcatggtggtgtgcacctgtaatcccagctactcaggaggctgaggcaggagaatcgcttgaacctgggaggcggaggttgcagtgagccaagatcccgccactgcactccagcctgggtgacagagcgagactctgtctcaaaaaaaaaaaaaaaaaaaaggccgggtgaggtggctcacgcctgtaatcacagcactgtgggaggctgaggcgggcggatcacctgaggtcgggagttcaagaccagcctggccaacatggagaaaccctgtctctactaaaaaatacaaaattagccaggcgtggtggcgcatgcctgtaatccagctactcgggaggctgaggcaggagaatcgcttgaacccaggaggcggaggttgcggtgagccgagattgcgccactgcactctagcctgggcaaaaggagcaaaactccgtctcaaaaaaaaaaaaaaaaaagacatttgggggatctggaaaatgtaaatggcctggatatgaggtaacactaagaaatgagttgatcatggaattgtattatgtaaagaattatgtccttattttgtcagggaaacatactgaacaacttagagatgagtgacactggctcatcagtccagggacagaagaggaactacagggtggcctcatagtttagggctgggcctgtggagggcatgatctcaaggtccctctggtgccaagctgctctgggagcagatgtggcctcacctcgctccccacttctcaatgtgggcaaagtccacccaggcccaagccctgcctcatgggcagggtgttcacagtttgggtaccaccaggagccccctttggccaatggaccaggccagcaaccccctcttggaggtgagcttccaggccccagcccagggtgcaggaagtggagctacacccattcacctctggccagggccactgtggggtcagttgcctcagccctggaaagctcagcttgtccccagggaacttggtcttggagagcagcctgccgtaaccgccccccgactacagctgcccccaaagctgtgaactcagagtatatgacaaatggccaagcacaagagtgtgaggccctcctatcctgcagaggctgctccagccacacctctgcagccaggaggcagtgacagcccagtgtcctcaaggtccccccatctcccaggatgcttcctgaaggctcaggttaaactgcaccagtggctggtgagcaaaggctgcaggcccttttcacaaaacactctgaactggtccatgcagggcaccctccctgtcttcctcccaacctactgcctgaccgtccccccgccctgcctttgcccaggtcatcccttcagccaggaacaatcccgtccctcccagccacaccttcgacagggtctcagccccaacatttcttttttaaggatgcacaccctgcaaatcccaggacaggactacagctctttttggaatcccctttctcatgtctgggatcatttgatatccctcttcctcaccttacaagggtagtgactgtgtttttgtcaaaccctgtgtccccggcactcagcgcagcaccagacagggaggagctgtgtttggtttatgtttgttgaatgaatgaccacatcacatttgcttggagggcctggccaaggcccgtacttcagccctaaatgatatggtcagggccagtgactgcactgggggctctgaggaagcagaactccccaccccactcttttgcctcacccctgccctggggggcacttccctctccctggcccccaccaagtggcctcccactgggactcctcaggccttgctgcagtcagctggttacctgtccatgcctgctttgagagggacatgccccatgcccactgagggtaggacccacatctcatgcagtgccagtactcagtaaagggcaaacactgagggcctgtaaccctctggatagtgacaacatagaggcaggaagcaagggacttcaggaacccaaaggaaactgggaaaaccaaacctcctttctcaatggagaacctgctggtcgctgtcctcggggagctagactcttgtgcacacacaattcctcatcagaaacaggcaaagagggcactgggcctcccctgctgcagctgtgtccacagagaacagctaggccaccacacaaccccaagcctggccctgcctccagcctcctcctgtgccaaccaaggcctcaccatggctcaatccacatggcccccagaggcagcagtcctggttcaaattcaggatctgcccttgctagctgcatgggtgagttctttcacttctctgtccctgtttcctaatgtgtaacacaggaataagcagtggcctcttccttccagggtttgctgcaactgtgcctgaagctctgtgacaatgcaccccccatctctgcaaaagcaaacccccaaaggcctggtttctaaagcaacagcagtttcagcagcaccgtcagagaggcacttcaggccaatcctggaggagccaggagtgacccttagagtgggccccagggacgtcagccttcttggaaaacagctcaaggggtgagggggcctccctcctcctgcctcccccttctcccactcccaaagcagccaggtccctagggagggtcagagaacagatgctgggagtttccagtccccctaaccagagggggtcacaaggaagatgtgcagaatgaacatcctgggaaactgggaaatgactagggaggaacatggtgcctcccccccagcaaaaaaaattatacccttccccatgagatggagtgtcagcaagcttccaggccccagcccagggtgcaggaagaggagctacacccattcacctctggtcagcatgctcccaacgtctgagacctcatttctcattccttcttcagtccccattctcattgtggttcgaggtctttctctctgagccaggaactccgcaaccttccaccctccctacctcttcctctcctaccccagcctggggctcagtcctggctcaagcactcagtccagcagaagcactgtgtagcctcccattaaagctcacgcctgtgaaaagaacacccattgaggccttgagatggggccacactgacccgctgactctcaggactggacacagcagaggccacacatactcagaacaaagcctggaaaggcaaggctggaggtcagtagttgtggcagcttcacatcaactcagctttaatgtgatttaatttccttctccctccagtgggccaaaggtgcaaagataagtatggctgttctctctccttctaacagtgaggtgctgggggtgggggtgggggaatatggagaagggaccctcaccacccacaccttcctgcctccccaacaagtgctgccctcctctgcccagcattctccccactttgccctcagctagtgggtgcttagcctccagatagcatgccccacctaggccctgccctgggcctgtgatccagaggtcccaagaagcagaggccaggctggatccagggggtcagccaaggtgagggtgggagcacacaggattatctcccagggacagggctgctgcctcgtagctcaggatggatagaatgtggggggatatccagctacattttccctccacaaaagaccagaatgggagggggatggggtgctgccccgactttcttcaactccccggagcagaaaaatgccctacctccactttccagtgccaagattcaagaagaaaggcaagcggagacttccctttctcagtccctgcttactaatggaaacacgggtccagaacctaaatccagtccctcctccttcataccaccgggagggaggtgcagcccaagcccccgaggccccaagggtccaggtgtaggaccctttatcctctccggcagccatccctgtgggtgtggcacccccgccacaccccattcttgtcatctcagggggaggggggaaatgtaatcggacatcccccccatccaatccatcctgagctgcgaggcggcggctctgtccctcggagataatcctgtgcactccccaccttcactcacctcggctgacgcaggagtctccggagcccgcactcccagacatcactgccctccttcctgagggtgctgaggctcggaggctcagagatgctactggtccaaggtcatgcagcgaggcagcggcaatgaacagggtcgtggggaggagggggcgctgaccccattacgcccccgccctcactaccgcactcatgcccccggacaatcgcttcgcggaaaacaccccagctgccaccagttaacggtcactgcgccccgggaacctgacatcactctagttcaggtgctggggagtcttccaggcccggcccccaacagcggagcccccccaccccagccctgccatcacggccgctggggtcccaggcactgactgctcaggacagggccgggaccggggagggggacctcggcgaacgggaaggaaccgggaggcagggagcagaggggtggcctcaccttgcggggtgcaccccggggccggggagggcagggacgaccactgcagcggcggcggctgcaggagctcaacgccgagcacgaggaagggagccccgcgccgcggccgccctcccgtcggcacgcccccgcctccgcccattggttgatctgggagggtggggcgagggacgctccggaccaatgagcgggctccaaagaacggccaactggcgagggccgcctacgtcacgtgccagggtcgccgaggcagcgccctgctagtccgcgcctgccgggcgagctctcgcgaggaagacgggcaggcggcccaactaggccaggggccagaaccgaccactcgaagagggagaaggagggcctcggataggccccgcccccgctccttcttccgcctgggggatagcgcctctagccttgaaccttgcttaggacgcacctcccttgggcccttcgctctcgggagggctgtcgggcgcgtctcggggctgggtggagctcccgaaggtggcctttctccctgggcttccacgccggcttcggccatcgatacgggccgtgttggtctcgttcaggagctgaggaaccctccatcactcctgtttcgaccccagggtttggacctcttccccttccaccccatcccctgtcttgaaagaagcaacccccgtgcgggcccgagacgcgtcccgggtgcctggccgggcctggagaagcatcagaacaaagaaggcacgcgggctgggggctgggagagcctgtgacgcgcccccggggaccgcagcctctgctcccggtctccatggaggcggtcgccatggcagcgagatgcgcctcgctcagcaccgcggggtgggatgtgggcgcctgcaatgagccgaggagcgagaggcgtggccctccggtctgcgggggttcttgccggtgctctccgcccgccggttcgcgaacaccccacctatacttcgcccgtggggacggattccccaaagtgccctcagtaagccgtccggagcacgcagcgccttgcttccaacggaactagagagacggcctgggcggccgaaggccagcctcccttcagcagggccggggtcgctgccttaaaggagccctcaagtctgccaccctgtggcccataacctgtctgctgatctccagtctgcacactgttggcaaattaatctttctgagctcttgttttcatcgcgtccctctcctgctccaaagccctctgggactgcctccagtagcgcttcacaaacttcagcagcactttgggtgactcatgtgccctcgcgtttgagagacagcgcttacattgagagcttttcacattctgatctcagcccatccactcctgcccactctacccactcccaccacactacctttagaccttgtcttgaaagtctaaagttggcagacggtgggcccaaagtggcccgtaggtgtattttgtttagctcacatggggctttttaaaagaagtccttccccctcctgcctcctccgcctctttctggaatcaggaatctggtttcgtggcttttgaaatatcagaagatctgacaacgtgggctcactttccagcctggcattacaagcccctttaagagcacataaatttaccatagtccccgctaatccctgtttctcacggttagttaccttctgggtctgtgcagacatttgtgatcccctccccctcattatcccaccttccaacctggaaggccctccccgctctcttctcatagccaaaattcaagccctcttcaaccaagataagttttgcctatttatttatttatttatttttgagacagagtctcactctgtcgcccaggcttgagttcagtggtgccatctcggctgactacagcctccaccccctcccccgccgggttcaagccattctcctgcctcagcctcccaagtagctgggattctaggtgcgcgccaccacacccagggctaatttttgtatttttagtggagacagggtttcaccatgttggtcaggtgggtctcaaactcctagcctcaagtgatctgcctgcctcagcctcccaaagtgctgggattacaggcatgagccaccgcgcctggcccaagcctgcctttttcaaaagtctttctagccaacttctcaaaaccatctctggtgtgggctctctcaaacacaaactggctgctaactgcacagcccggggttatttccgaattgtcaactcctttcagcaaacatttactgaatgtatctgatgagtaagttattgtgctaggctctgtggagtggaaggcactcacagtttagtgtaggagggacaccacacacaaagaactgtggtctcctgtccttgccaaatgttcctcacactcccaggcatcgggcagtctggcccactgggaggagctgaaatacagagctgcccatgcctagggatactacatgctctcagggaaggccaaagatggttctggaaagcctccaggagaaaattagactctttgtgtcaggaggaagtcagggcaggagttacctggctgccccatcactgcccaatgtgtctgtgttaccacacgagtagtgcccaggctctggggcttcctgtgcggagggccttaccaaagatagatggctctgcaagggaaaccacaggcccctaccaggccccctggagacagaggcaggacaagaagaatcaggaacaactccaggttaggagaacagatggagcacgtataaaacatccactcacggccgggcgtggtggctcacgcctgtaatcccagcactttgggaggctgagatgggcggatcatgaggtcaggagatcaagaccatcctggctaacacggtgaaaccccgtctctactaaaaatacaaaaaattagccgggcatggtggctgatgcctgtaatcccaactactccggaagctgaggcaggagaatggcgtgaaccccgggaggcggagcttgcagtgagcccgagattgcgccactgcacttcagctttgggcgactgagccagactccatctccaaaaaaaaaaaaaaaaaaatccactcaccatgagaacctggtgacttttaaaaaaaaacaaaaaaacacaacacttgctacaactccaaacacacggttttttgtttttttttttatttttcagagacagggtctcactatgttgcccaggctggtctcaaactcttggcctcaagcgatcccccgaccttgggctcctaatgtgctgggaccagaccagaggcacaagccactgtgcccagccctctattctttttaattaaaaattaaacaactataacctcattggaaaactgcttggcagtacctaataaaaatgataaatgaaatgagtaattccactttttttttttttttgagacagagtcttactctgtcacccaggctggaatgcagtggtgcgatctcagctctctacaacctctgcctcctgggctcaagcactcctcccacctcagcctccccagtagccaggactacaggcacatgccaccacacccagctaatttttgtattttttttttttttttgagattacaggcatgtgccaccacgccggctaattttgtatttttagtagagacgggtttctccatgttggtcacgctagtctcgaactcccaacctcaggtaaactgcctgcctcagcctcccaaagtgctgggattacaggcatgagccaccgtgcctggccaatttttttgtatttttaatagagacggggtttcaccatgttggccaggctggtctcgaactcctgacctcaggtgaccgacccacctcagactcccaaagtgctgggattacaagcttgagccaccatgcccagccatggcatgtgtttaattttaaaagaaactggcaaattgttttccaaaatgactgtacttacttacactcccattagcagagtataagagtttctgttgcttcacatcctcatcaacataaaaatttgcaattttgtaaaaaacgctacaccatgctaaccaaacaaagcagggcaacggactaccagtcttcagcctctcctctgagccatgcccttgtagaggacaatttcacaacagctatcaagattacaaatgcacacacaaaaaaagagaaaacacacacacacacacacacacacacacacacagccaacatggtgaaaccccatctctactaaaaatacaaaaaattagctgggcgtggtggcaggtgcctgtaatgccagctacccaggaggttgaggcaggagaatcgcttgaacccgggaggcagaggatgcagtgaggcaagatagtgtcattgcactccagcctgggccacagcgcgagactctgactcaaaaaagaaaaaagagagaggaaaaaaaacacaacaaagattacagatgcatatattctttgatctaggaaccccagttctggaatttatcatttagatatacttccccatgatactaaatcatgagtgtatgaggttatttattgttgcattcttgtgatagcaaaagattggaaataactcaaatgtccataaatagaagacttcttaaataaatttggtacagacaatataatgttatatggctttaaaaagaaaaaaaaaaaagaggaagttttcaatgtcctcccaagtagccaggattacaggcgtgagccaccatgccgggataatttttgtatttttagtagagatggggttttgccatgttatacaagctggtctcgaactcctggcctcaagcgatccacccacctcagcctcccaaaatgctaggattacaggcatgagctaccgtgcccagcctgctaaggaaagattttgtattagctaggattctccagagaaacagaactaggaaagagggcagataggaaagactgacatattataaggaattgactcacacaattatggagtctgaccagtcccaagagctgcaggttgggttggcaagttggagacccaggagagccaatggtttagttccagtctgagtcccaatgcctgagagccaggaaagtcaatgatgtatctccagtccaaagggcagcaggtttgagaccaaggaagaatcaatgtttcagtttgatcccaaaggcgagaaaaaagctgatgttccagttcaaagaccaccaggcaataaaaattctctcttatttggggagggccagcctttttcttctatttaggtctcaactgactggatgtggcccactcacattagagagggaaatctgtttcactcatctgctgatttctttcttttttccttttgtttttgaaatggattctcactctgttgcccaggctggagtgccgtggcatgatcttggttccctgcaacctccacctcccgggttcaagcgattctcctgcctcagcttcctgagtagctggggttacaggcaatcaccaccacgcccggctaatttttgtatttttagtagagacggggtttcgccatgttgaccaggctggtcttgaatgcctgacctcaggtgattagcccgcctcggcctcccaaagtgctgggattacaggcgtgagccaccgtgcccggcctcatctgctgatttaaatgttaaccttagccgggtgcggtggtttacccctgttttcccagcactttgggaggccgaggcgggcggctcactcgagctcaggtgttcgggtcaacatggtgaaacctgcctctactaaatacataaaaattagctgggcatggtggcatgagcctgtagtcccagctactcaggaggccgaggcacgagaatcacttgaacccaggaggtggagggtgcagttaactgaaattgtgccactacactccagccagggtgacaaaacaagactcttctctcaaaaataagagtaagacaaatgtgcatgtttgtttatagtcacataaagtcattccggaaggatacataaaaactaaagatagaggttccctattagagatgtgctgggaataagaagacttttcactttttatgtgttttttttaaatcatatatatgttttatctattcaaaacattaagtaacaaagtttaaatacccttgcatgccttcctctcctgtagggttgtgaacactggagggaacaaatcatgttttatactcacttttgtattcctggtatttagtacatagtaggtactcaataaatagaggaaagaacgaatgaatgaaccagaagatagcagatttagtgtcggttagagagaggagaggggccaggcacagtggctcacacatgtaatcccagcattttgggaggctgaggcaagaggctcacttgaacctaggagttggagaccagcctgggtaacaaagcgaaatcctgtctctccaaaaaaaaaaaaaaaaaaaaaaaaaaaaaaagcccaggagcagtggctcacacctgtaatcccagcactttgggaggccgaggtggggtggatcacctgaggttagcagttcgagaccaactaggccaacatggtgaaacaccatctctactgaagatacaaaaattagttgggcgtagtggcaggtgcctgtaattccagcaactcaggaggctgaggcaggagaatagcttgaacctgggaggcagaggttgcagtgagccgagatcatgccgctgcactccagcctggatgacagagtgagacatctcataaaaaaaaaaaattagctggatatggtggtatgcacctgtcctcccacctattctagaggctgaggtgtaaagattgcttgaacttgggaggctggggttgcagtgagccaagattgtgccactgcactccagcctgggcgacagagtaaaaccctgtctctaaaaaagaagggagttagggctgggtgcggtggctcacggctgtaatcccagcactttaggaggccgaggctggtggatcacctgaggtcaggagtttgagaccagcctgaccaatatggtggaaccccgtctctactaaaaatacaaaaagtagccgggtgtggtggcgtgtgcctgtaatcccagctacttgggaggctgagacagaggaatcacttgaaccagggaagtggagagtgcagtgagccgagatcataccactgcactccagcctgggtgacagagcaatactctgtctcaaaacaaaaaaaaagaaagaaagaaaaattttttaaaaattaaattaaaaataagggagttataatcttatctaatgtaatcacaaaagttgacatcctatcacatttgctgtattctacttatcagaagcaagtcactaagtccagcctacattcaaggaaagggtacacatgggggtgacgtccaggaggcagggattactgggagccacgttaggagctgtctactacaaggaggaaaaagcaatgaaaggatgaaatctaggatggtggttatctctgagtggaagcaagggggagagatgcaagaagcacatggccggtgaaagttatttgtaataattccattcttggggcaggtggtggatttataggtgttcataagtgagttgattatggacaaatggggagagagtgtcatgaaccaaggattatatttagtccaattcagtaatatttaaaatattcaataataaatgaaataaaaattgccaactactcctgaccttgttctgagccccatccatgcactccacctggtcttagtttaacactggctccacctgaccctccacttgccccaaactaaccgttagcccttcacacacactgacccttgctatagcaccacgatctctccatagtctgcaataggcccccggagttctattccctgaaacttttatccatgtaacctcacctgtaggctgctatatgtaaagatagtaggaatggctgatggagtggcccgaggaccaggagacagggcaggctaacagttctgccttagcagtagcactgtgggcccagaagacaggtttgatggctggtatcacgacctcacctattggcctcctacatggcaaaggctgggccaggtcctgatggttcttcctctttgaggcttctgctcctggtatccagatatagctaagtctgtctctgagaagtgcacctctgccacagcaccaggcctgtaactcccttgttggtaatagactctatcaactctctcctttcctgtctttgatgaagtaagctaccatactggagatggctatgtgtcaaggaaatgagagtggctattggccaacaatgagtaaaacactgaagctttcagtttgacagcccactaggaagtaaattcagtcagcaaccaagtaagcctggaagcctggatccttcctcagttgtgccttcagatgagactgcagttctattggccctttgcttgcagccttgtaggaaacattgaagtggaggacccagccaagcctggcctggactcttgacccacagaaaatgtgagatataataataagtatttgttgttctaagctgctacattggtgataatttgctacacagtaagagataatgtatacaggttcttttcatttcttttgctattacaaagagtgtttctgtgggccaggcatggtggctcacgcctgtaatcccagcacttttggaggccaaggcaggtggatcacttgaggtcaggagttcgaggccagcctagccaacatggtgaaacctgtctctaccaaaaatataaaaaattagccagatgtggtggcatgcacctgtaatctcacatacttgagaggctcaggcaggagaatcgcttgaactcaggaggtggaggttgcagtgagccgagatcgtgcaattgcactccagcctggacgacagagcaagactccattttaaaaaaaacaaaaccaaacaaaaagagtgttgctatgaacgttgttgtaacatgtctcccagaacacagaacacaagagcctaggtttcccttgggtatgtgctgggtatatgcctaggaatggaattgttagatctcaggatgtttgttttgttttgagacagagtgtccttctgttgcctaggctggagtgcagtggtgcgatcttggctcactgcaacctctgccccccagcttcaagcaattctcctgcctcagcttcccaagtagctgggactacaggtgcccgccaccacgcctggctaattgttcgtatttttaatagagatgggttttcaccattggccaggctggtcttgaactcctggcctcaagtgacccacctgccttggcctcccaaagtggtgggattacaggcctgagccaccatgcccagctggatctcaggatttacaaatgttccacattactggtagtcagaaaaccattaaagttagggtcatgccatggctgtagtgtgttggccagggttgggagtaaagcctggcctcaatccatggaagttgggattgtctgtgggcagggttggagtcgtacagagggccgagttaggggtcagtccttacgtagggttgagggtgagtgtgtggccacatttgggaacaatttgaagtctggtttggggttgggtttggacaaggttggagaacactgtgagggtgggtttgggggtcaatctaaggccagggttagaagttaggctgtcatcctgtgcaaaacctgacccctggtgatatcatcaacctaccaagctgtggccgcacaggacccagccacccacaagatgagatccactctgggtccagaaagctctttcactcaaggcgggggtggggagatggaggacaagtgaagaagaaactggctcccctcagatgcaaaaagagaaggcaggcacaggaaatagaacacaacactgactttaatggggcagccctgagccgtaaccttcaaaccttccgcctcagtaccccgtgcgcgaggagggaggggcgactgctacgggcacatcgtcgatgtcctccctactcctggcgatcccacgcctcaccccttcctctccagctgctgcggtctccgtcgccgaggtgggtcccaggtaagctcccaggaggggcaggggagcccctcagcggcccgggacagattcccccatggctgagggcatggggaactagcctggatggagacgccgccgtcctcggagctgggcggggacctgattctgggggtgtggacagagccaagggaccgccccccaaggcccagcgccgggagatgcaaggccgggcccaaggtgttggacactgctttgggggacaggctaggtctctgcacgtggcttctgggctctggaaagcggtccattctcctgacccggatctccggagtggtaggaggcggctcagtcccgggcctgcgctcctagagttcctgtcccatctcctcccacgctcacccatcccaaggaaggagggcactcgggccccagcaggctcgtgagagcagcgggctccgccctcccaatggtctatccatcggtgggtgggtccggcgcggcgtcggggctctggcgggtacccgggcgtccccgcgcggcccgggccgccgctcaccgctgcttacgctccgcctgctggagccgccgggagaggtcttcgatccgcacattcttgagctggagcaggtgctccaggcgccttaccttcatctgcaggacctaagccgcaccgcgcaggcgtcaagcctggcggtctgctccctcctgcccggcctctgctggccgcgagcccccacccgcagccttcatagacccgtcacttgcacggtctcttgagaggccaacagctcctgctccttttcagcgatctggaggacgaagctggggtcgccctgcaacgcccggttatacgctggaggccgaggcgccggcggccggtcccagctggggtgggaagcagcttagcggaggcttggacctcgggccctacactcacccttccagggccctagaccaggccttcccttctccccctctagcccctttcctgcctcccgtctcaggctcagcgtaccgtgccccccaaccccagctgtgtgcagagatcaccagcctggtgaagtctggaactcccagcttctttacctgagctgaccccctcccggggggtccgggacaccttcacctcgggccttctgggatacacctgagacaaggcaagtggaggaatgactgtgctggagtctcattctcctgcttccccctccctacttccactctcacaagtgacaggaagaaggacagaaggaaagctccaaccgggagggagaaatggaaaaggccaccttacccacatccatgtagccactgccatcctggggagccagctcctgaaagaaccccgggggtccgctgtaagcccgggctcagtcatccccaaggagccccatgtcccttccacgggcccccaggccaggctctattagccaagcacccctcccccacacttatcacccaggctggcctttctgtggtctggggagcgccccccacccagcccccagagcagtgggagaagctggagcggggtggcacctgtaaggagccggcgccctgcttcctgcgcctctgcctctcctccaggcgctgcctcagcgggatgagcaccagctccaccacgcctggggcgcactgcgcgatcttgcgcatcacgtcatccggtactgaaaagttcagcctcttcagtaccttcctgaggggtagacattgggatagcagattgggtcctggggaaatgaagcgaggggatgggggagagggaagggggcatggtggttgctgttctgagtgtacagccctgggggagtgaaagcgcatcgcatctgagcttccttgtgtgggtgttccgaggttccttgcctccttcctaatagaagagtctcttgattttgtaatcggacgaattctgtctgcgttgaccctccccaccctgccaccaaaacagtctcagcctgggcaaccaagaccataggagatgatggggctgcacaggtattctgctacctgttcagatgaccccagttgctgagcttctgctggagagagttggcggggacataattgtgcatctccaccatcttggggaagtaaaacttgatgacctctgcaacaaggactgagggagaggggagcagacatggaaagtgggggcatgggtaaggaaggaggaggtgagagaaggtgggggtgggagaatcaggcatggcagagtggtggaacccaatggaggggtggagatgcggaaaggggaaggataaggagggagacaaagacattaagattagtgataggtagaggagactaagtctccagcatacctcagaggagctgcatcccttccccactaatttaggaatgggaatactgagtctctggtcctccagtagagaatgagtcagccagtgcaggggctcacacctgtaatcccaacactttgggaggctgaggcgggtagatcacctgaggccaggagttcgagaccagcctggccaacatggtgaaaccccatctctactaaaaatacaaaagttagccaggtgtggtggtgcatgtaaacccagctacttgggtggctgaggcagaagaatcgcttgaactcaggaggtggaggttgcagtgagctgagatcatgccactgcactccagcctgggcgacaagagtgagactctgtctcaaaaaaaaaaaaaaaaaaaaaagagaatgagtctccaagagaagctttcaggagcccagggaattcacatggatgcatgatgcacacacacacacacacacacacacacacacacactccaacaccaaagttccaggagagtaaaagactcacgtaagcagagacacatgtgataagatccccctacacatagatacatagggcagaatgtgtagacacatagaacaaacacatgctagacacacataggcacacatgtagaacacacatagacagaagcacaaaaagccacatatgcacacagcctccccaaatatacagatacagagatgcatagaagtacttgacatgaaaagatgacagataagtatttctgtcattgagatcccaggagacattcctagaaacacaaacaccttcacaatcacaaagaaacacatgcacacaagacccccccaaatacaagactaaacatgtcaacacatatgaacacacacatgctcagactcccagaagacatagaacacatagcaatgagtgagcacatacagaaatgcataaatacatatgcagacacatatcttaatcattaatgtccctggagggagttagaggcaccccccccaacatgcacacaggatagaacatgtagacatacatatatggacacacatcgttcacacacagatgcaaatagaaaacaagcacacacatctaaaatttgaagtcccaggaggcagattcctccccactcccacaagcacacaaacacaaagagtttgcacagagacccgttacacacgaacatacaggacagaacatgcagacacacaagggcacacacctccatcgctaaagtcccgggagaggtttcgcttgggccgggacagagggatgttgtctacccacaggtacagctggtgcagcgcctcctcgtccacgctgctcgccattggcgtcctcacggcctggccgccccagcggtgccgggtcccgccccagcctcacgacccttcaggcgcttcctttctcgccactgcagaagcccataactgcctctgcctgcctaatccagagactcacgtcccagctgggagcggccatattgttttctgaaaccatggaaaccattcccgactctcctgggtacaaagacttctgggagttgtagttttatcccctccccgctacctcctctcaggcccgagctttgaaaaggcggttagctgcccttgtcttcttcccaagaaagcatcacttctgtgcccacgccacctagtgaccagccactcatccattttgggaccagccaaagccaaccaggaccccagaattccgcggatccttctatagtgtcacctaaatgtcgacggccaggc

SEQ ID NO: 6 - DNA, 23574 bp, Homo sapiens
gctggaccttcaacagagaaggatggctggcagagtcctagtctgataggcgcccccttcctagtgcctctggagcaggagcacagtgaaaacccagctgtctgcgctgatacccctgggaggagcctttgctgcagcaactccccgccctcattaagtctccaccccaggctgcaccgtcaggtaaacctgggagccacgggattcggcgcctgaatgtgcctagacggaccccaacacactagccctgccacgcacacccagcaacacacagacaaccacccagggagacctggtctctcccatgctaaactaggaacgctatgaaacagcatttttcttttccttttttatataattctattttaaacttaaaaaaatttttttgaatagagttggggtctcgctctattgcgcaggctggtcttgaactcctgggctcaaaccatcctcccacctcggcctctcaaagtgctaggatgacaggtgtgagccaccatgcctggcctattttaaacttcttattgttgactaattttagacttgcagaaaagttggaaaatgtaagcattttccttacacccttcacccagcttcccctgatggtccatcttacttaatcatagtccaatcattacagctaggaaactaaccttggtacaatcctgttaacgaaactgtagactgttttgcatttctcatttttccactaatgtcctttttctgttccaagatccattctaggatcccacatcacagttgtctccttattctcctcaatctgggagttccttcatctttccttatctttacccttgacacttttgaagaatcctggccagttattttgcagaatgtttctcttgagttgtctgatgttttattctgatcagaatgagacacagcattgttttgactaaccaaaaagttattctataagtaaatattgaggttaaaaatctcatccaacctgggcaacagagtaaggccctgtctcaaataaagtctcaacactaagatttaaaaagtgaccagaaaagcccccactatgatttgtcttgacttttttttaaaaaacaaacaaacaaacaaacaaacaaacaaaaacgatgtcttgctctctctctccctcaggctggagtgcagtggcacgatcttggctcactgcaacctccgcctcccaggttcaagcgattcttctgcctcagcctcccaggtagctgggattacagacgcccgccaccatgcccggctaatttttgtatttttagtagagacaaggtttcaccatgttggccaggctggtctcgaacttctaacctcaagtgacccgcccacctctgcctcccaaagtgctggaattacagacgtgagcaactgcgcttggcctcattagcatcttaaatctccacacaggggtgtgttccttactgttataaggagcaaaggatcagtttgaggacaggtaaaataaaaatgcgcttgctgcctagagggagaagtccctgctgaagatagctttgcttgaatgagctcaattgcaatgccagtgctgaggcttgttgactgtacggtcaccacagttgctgctgcgcgcctagaacatggtcactttcttgactacctatcctgtctcagtacatctgtctgtggtttgtggtggtccatttcctaatttttttaatgaatcagaagactgtgatgtgctttccgctgtgctaaccatggccgctgaagcaaaatgtaaaccaagatgcccctgcagtggttgtgcttcactctacgacatctgttaccggaaaggggtccagattcagaccccaggagagggttcttggatctcgtgcaagaaagaatttgagacgagtccataaagtgaaagcacatttattaagaaagtaaaagaataaaagaatggctactccatagagagcgcagccctgagggctgctggttgcccatttttatggttgtttctggatgatctgctaaacaggggtggattgttcatgtctcccctttttagaccatatatggtaacttcctgatactgccatggcatctgtaacctgtcatggtgctggtgggagtgtagcagtggggaccgaccagaggtcactctcatcaccatcttggtttgggtgggttttagccagcttctatattgcaagctgatttttttggttggtttggtttttgagacggagtctcgctccaggctggagtgcagtgacatgatctcagctcactgcaacctccacctcctgggttcaagcgattctcctgcctcagcctcccaagtagctgggattacaggcacacaccaccatgcctggctagtttttgtatttttagtagagatgaggtttcaccttgttggccaggctggtctcgaacttctgacctcaggtgatccgcccacctcagcctcccaaagtgctgggattacaggcgtgagctaccgcgcctggcctactgcaaactattttatcagcaaggtctttatgacctgtatctcctatctcatcctgtgacgcagaatgctgtaactgtctggaaacgcagcccagtaggtctcagccttattttgctcagcccctattcaggatggagttgctctggttcacaggcctctgacacatcctcttgtgttttggcgtgtgggaggaaagaggggtgagggaaggaaactcaaaaccaagctctgaccacacagggcaggtacactctcccacctgtctgtgggtgccacaagtcaagggaggggcagagagagaagaaggtgtgacagatggccgcaggccacagaatgtcagaggaagcccagttcctcccggggcagcccaagtagctggtagttgggtggccaaacagagggcgtcacagctgagctgggctcgctcgctacccccagctcagcgtccactctgcccctcagtacctcctgctcagcctcagggtccatgcctaccctcctgcttcccagtcacttctgcgtgcctcctgcttttctgctgttggccccatgccagctcctttctgctgagcttctcttctccagttctgcagcacagccaggtgatcctgggctccagacaggcctctcccccagtctggggcctcccctcttagagccctctttccttcccacgtggcctccccagggttcgccactgaatggagaaggggtggagggggtgctgggcagtcttggggattagccaagagggcagagttggcctccccagggtcccttgtagctggagtcccgcggggtctagacaccccctcctgaagggtaagagcgggggaggtatattaacgtgtatttttagagtctctcttttttttttttttttttttgagacggagtcttgctcttgttgcccaggctggagtgcagtggcacgatctcagctcactgcaacctccacctcccatgttcaaacaattctcctgccgcagcctcccgagtagctgggattataggcacgtgccaccacaccgggctaatttttgtatttttagtagagatggggtttcaccacattggccaggctggtctcgaacttctgacctcaaatgatcctcccgccttggcctcccaaagtgctgggattacaggcgtgagccaccatgcctggccttagattctctattttatgatggtttaacatctcggggtgggggcttgttggctggagagaaactgcttgattcctggagatcagaaacaactcatgcctttcatatgcaaaccgaccagtcttgagtccatacaccaaccacccccttcaaggaactctcacatacgaaaccagtatttcccctgccctaaaccagctcagggccaggcacctacccagacaattagagcccaaccccacgccccaaaccccaccagaatgattcaaaatgccaatcctaccctcttcccctggcctgccttgcctccccagtggaaacggcactgtgggctgtggcctgtgccttccactcgctcctgattctgtccctggaccaaacctagtgcctccccactgtggccctgcatggtgggaaactgtgagtaataacttatttcaacagcattggcctctgtgtcatcagtcaccttcataaattaaaattttgcaactacaactgaggcaggagggggctttagagagcactctcacctggccccttgggcttgtggagtggagcccgaggtgaagttgcctccctgcactcagttttgggatggttttgttatgcggcaacagctggctgatataggccactcagcagctcttgcatgaagcaatgggagaatgtgaacgcccaagggaggcaggagtgacagagcaaagagggtgttcaaactaggcaacaccgtcctgtgcccagacacatgcctggggctctggctgccatctaggctcggcctgccagccaccaccccgtcagccaccaccccaccaaccaccaccccgccaaccaccaccccgtgtggtcctcagggcaccccatgggttgaatttacatacagaaaagactaaccaaggtccaagttaatatgtctttttaaaatttattttagagacagggtcttgctctgtcacccaggctggagtgcagtggtgccaccatagctcactgcagtgttgaactcaggctcaagtgatcctcctgcttcagcctcctgagcagctaggcctacaggtacacaccaccacgcccagctaattttaaaatttttctgtagatgcggtgtcttgctgtgttgcccaggctggtgtggacctgctggcctctggtgatcctcctaccccggcttcccaaagtgctgggattataggcatgagccaccacgcctatagccaacatgtctttcttttgacttctactttggtatcttttcttaaatggttccctctgtccccccgacacacacagaatgggggagaggctgtcagattctgagctccagaacctcaggtgtagcactgggattgggggtgggggctcaggaaccacctaggggagaagacagggtgggaagaaacaggaaggaaggtccccaaaattatgtttgtttgcagaggccagccaggctgcaggggagtgtggactcagtcgaaccatagggccccaggaccactagcttctggccagcagtcatgccctccacagagctgggtccgtggaaattgcatgtaggagacacaccagactcccaggacagagcccttttgggatggccagcactacccagcctccactggtgagggaggtcaggggctgtgtgacctttgcttctgggactgatggtttattgagctggagagtgtgcccagcagtgttctccagccctcaggaacttctaatgtggctctgggttcctggagtgggtgggtcgaagctccactcggggaagaaacttccaagctgcctgcaggtgctggaggtccggtgattcactggctctgcccctgcagttcaagttcctggagtggctgtcagtggccacctgtctttaaatctgttcattttaggagctacctctcaccagaggcaggatcttggcatctggacttgatctgctgagaatgaggaggatatgttgtcccctaaggactggggccccaggctgcaagctgtgtggcagagagcccatcctcactcagtgaggaccagtgatccaggaaaagccacagcttctccctccccagcccaggggcttccagcatcctggtctccatgataaccaagaggtcataaactcatttccataataacctgagcccagaaacctgattagggggcagcaaactgaggggtgggagaggtgggagggtgggcgatgagagggggaggctttgaatccaggtccctgcctaccttgggggtcaggcgagactgctggcagaggcttctcagggtggctgctgggctcatgagagttctcagggtctgggagaaatggtggagggtaaatgttgtgaatatggtcagcaggagaccctggggctggggaggggcataggggactcaaggtgactgggtgctgcccatctggaaggaggcaggaggcatgagcccttcccttctcccttccctctccacctccccctggtgcctcactcacccaggggccagggctgtccagtggctgtggggcccaactccatggggtgaacgccgcccagggggtggtccctgtgtgggccatctttggggctgagcaacgtgataagagtccaggaggttggcacagtgatcctgagtgggttattgcctccccgcagcatggtgtccagcccagggagttctgcgtttactgagtttcttggggcacccatctgctccaagtcaccctctcagctcccttcctgctccctcttcaggggagccttgggatccaggctccaagtgagcctcatgccctcggctggcacctcctctctctagtcctaacatttcctccaggctctgacaccacccagcagcctggcactctccagatgctggcatcgctcagcttccaaagaaccttggatgtccgccccttcggcagctatgtctgctctccttgcccctgggtgccctgctgcccttgatgattccaagccatctttgactgtccccatcccatcccccaaggccttgtcatttcctgtgatgttccttcaaaacattctcccctgccctgagactcccgcctggggatgagaagcagccgccaccctctgcagcgccccctccgtgctgacacgccaggctctggccaccttgctcctctgcccacagaccctcaatcacaactcgctttgtcagggtcctcttagctgccacccggggcccaggtggtgccctgcccctgtctgttatgccctctgcccccatctctggcccaaatcatgccatctcccttggcttgcccggagcactcccaagaccaggctatgtcagacatggccacagagtgcctgccctgcctagggccctggtgcagggtgagtcctaggacagccatgcttagtattatgtgactccccactccgccaccacccaggtcacagagaactgggttaaggcagggccctggcacaggggcagccagcaccgcagctgaccagtggtatggagtgaaaagatgtgctgggcccagcatttgggaacttcaagggggtgacagaggtgatttgtgcagaggaagtggcaaagggccgaaaactggtgagacagaggctggacaggcctccgggggcagcatggtacagggactgcaatctgagccagggaaaaacagggcgaagtcaagggtgaggcagccagctggtgggagaagcaggagagtggacaagaggagctgtactgggaggtagagggccatgccttgcggtgctggtggtggggcagggatccaccccctcgcttgactgggaggccactggaacctcctgttcaaagctacttctttccatggcctctggggctgctctctgcacctgggggcaaggctgagggcctgccccagctcccacagccccagcagagctctgaggaggggaaccgcaggagtaggctcaggaagcaggcgctcggagcctacccactgcacgcagggtcccttctgcagccccagctgcatcgctgcagatgggctcctgggagtcggtagcaacaccaggccaggccggcccctgggagcagaggcagcaggacgctgaggagcatggccagcaggaaggtgtcatggtctgcggggattgggggaaggggcgctgagtcctgagcaggtgcaccaccccagctcctgcccacatgccccccactggcatactttcagcctgcacagggccactgtccatgctgccaccaaagcctggcttgtcacagaagggtggagcccagcctggagcacagtggcagttatggttgctattgcaaacctgcagagaagagaagaggagggtcacgtaggattaggaaccccaaggtcacccccactcctcgggctctcaccccgtggctgtggcaggcagtcaggcagcgctgaagctcctggaaggcattcttcctgcagcgcctgctctggcacacctacgggcagtgcaccaggcagtgagggggacactggcctgcgggattcaaacggcaaggaggggtcgggtgggcagagctcaccattctaggtccacactgggtgcctggctctaccaggcccaggccaagcaggtccagctgggcactggggagtgccaaggctccccgacaagtcacttcctggccatctaggtgaacggtagagtccactggcaccatgtgcggtgcgagcaggctgggctttccaccctggcactgcagcttcccacacagggcatccctggggaggaagtagaggggggtcaacagctgcagtaccccccttccccaaacccactccatagcttctgctccctccactcagctccactccctacctccctgcacagggcaggaagtggccctcgctgtcctggccgcagtttccatgagcatctcccgcagagttcaccacctggaaacaggcctcgggagctgggtgggagcctgaggaagcatgggccaggctgggggcagctcggagaggggctgcgctcagcgggcctcagtttcccctgcccatcttccccacagtagaaaactggccccaccagaggatggggggcagggtgcaagggtgctcgtgtcctctcaccaggcccccagagctgctggcactgctgctccagcgtgggacatgcgccatcccagcagtagccactgcccctggcacagggtgagccgtccagtaggtaaacgtctgggggacagtgggaggaggtgcccgtgcaaaactcagggaggtcacagtcacccatggcctggcggcacagcgctccagccggcttcagctgcgcaggtgacgggtggtggggaaggcagagagaggccacgtgcagtgagaggtccatgccgagagcgcggctcggagctgggggagccaggcctacccaagcccagcacccaacgggggaacctgagggcaccaattaactaaggccaacaggccggctcccaagctccccgaaaccctcaccctgaaccttccatgccctcaccaggcagcgcacgcagcagtccccgtgggcgcactgggcccccgggcgcagcgagcagttgtgagcaaagcagcagaggtcgcggcactcctgggaccagaaaggcaagaagggcccaggtgagggcgcagcgccccagacctgagcggagagggcaagtgggggccgggcgagccgacttaacctggccagggccgcagtcacactcctcgcccgcttccacgaagccgttcccgcagagcgccggcggcaccgggagtccggggtccggggcattggagaggcaagcgccgccccccttgcggaagaaggcgcgcagctggcggcggctgcaggcgctgaacacgcgcggaaacgggtgcctaccggcacggggagggcattgggcatggagggacagtcccccaacccccgcgcttctctgatccccacccctgggcttggctacagccgccagacgcgcagagcccagagaggggaagtaacccgcgcaaagtcacacaacaagcgggacaggggacgatgcggccccaatagtgagcagcccgggacccaaggtggaatcgcgacccgacggtgctcctcccggtgtaggagtaacctcgccaggttactcggaaaataatcttcataccgttgagaatccactttgcctgagcttcttccctttaagcctcataaaccaccctgaagcggacactatgatcattatccccattttacagaagaggaaactgagggacgaccaaagaaacgcagcggaggaagtccccaggactagccgccccgccgcagccccgaccccccacccgcgtacccggtggccgcagccatgacgcagcctccggactcggccgcagcctccacgcagcagccgtcggggtcgtggctgaggccgaggctgtggccgatctcatgggccatggtggctgcggcgccgatggggagctccgagtggtcctggggggccgtgggagggcggtcactgcggccgtagagcctcctgtctctccctcgcccccgcccgcggggctcaccgtgctcacgcctcccgagctctcggcgcggcacatgccctcgacgggcgccaggcccactgtggcgccctggaaggcgcggcccctgggggcggagcgcggcgtgaccaggcggggccgggaggtgaggccgccccacccgggacccgcgtccgggtcagaggcacccacgtgagcagctgcgcggagtcgtggggccgctgcgcccacagcccccggcgccactgcaggaaggcccagagcgtggcgttggcgtcctgcgtgacgcggctgcggtcccgctcggtccacacctccaggccggtcagcgccacctgaatgtccagagtcctgagaagctgagggcgaggcggggctgaagccgggacagggcgccccatcgcgccggtggtccttcgtggggcgcccttcctcttccccaaaccccaccagcacctgcctgtcctgccgccgccacccccatcaccgctctctccccgccgcccccaacctggtccacgtagttggcgacttccaggagacgctgtttggtgtggttcaagtttcggtgccgagtcaagaactgggaaggcagaaatcccggtggcttgaggggctgagctggccccatccctgaccccgccaacccctggggtctctcctcaccagggtgtggtctgccacaatgtacagttccaggtacttccgggtcctgcgcgcttctcgcctgccctgcggaggtgcaaatggggaccctgagtggaagctgctgggcttgagccctgacccccaaccccagctcccagaaggaagtttaacatgttttctggaacttgtttcttcagacttcaataaaaatactgggactcgaggcctgtgaattcctgtctcttctgatttggagggctatagatacagcattcccactcccatccgatcgatgcccctgaccctgctctggggaccaccaggaaggctggtcatgcccgctttgttcccaggatccctgtggccacaggttcctttccaggtgagcagctgctccatccgaaagatctcgtgggttgagaagtccttggagccccggggtggccagggacgcagataatagctggcattcctgctgagggtgatcaggccactagggtgcagaggggtaggagcgggtgtgagggagctctttccccatcccaggcccagcctcctctcccagagctcacctcatcccagagcaggtgcagaggactacccaggagtcggggaagccccttactcgcccttggtagtggcaatgatcctagggaggaaggggccagccccaaatctcagccagggctggagcaagaggggcaagagggagggtgtggtaggggctggctccaaccgccccttaggaatgcaaggaggagtaggggtaggaatggtgggggggtacctctggcggtgcatcccagagcccatggaagcatctcaccgtgtggttgggggccagcaccactggctgcccatctgggccgtagtgggtttctatgtatcctggggccagcagcctgctgagagggggtgttacagggaacactgaattcagcttcctcctgcctcctccaggatgtctcccagccttcctccctaaatgctaatggagcagctttatgagtgagacactcacagtgtgtcttagggaagggacaggagcaatggtgacttgctcagatcagaaactcttggggctagaggaaggagccttggtgatggcttagttgtgggagatgtgaatatgggaagcaccagggaggacgccggggaggagtgggaataggggaagagtttgtggttcccaggggacctgcagcaggcagcaggatccacaggatcgggaggggaggagtcaggagacactgccgaagaatgggacttggagttggggaaatgcggtgacctcccccagttcccctgcctgctgccctcctttgttgggcatctggtcgaccctcttgcccccacctgccctagatccttgaaatattttcctcagacttctagaccccacatacctcccacctgtccttcagtgattgatgctcaccccctgcctccagagaaaacagaatcgccacctgcccacgctgcttccaccctccctgccttctccacacccactccggtaatgattccatcttcaggctccatctcaacaggatctttcccacacggatggatcaatcataagtcaatctgtcttctttaaagaaaatccttaacccaacctcaccttggcctcattacctccagaccacccgctaatgatggctgcttcccccctcccaggcattccaccacctgccccagctctgccccctacccctgccccacacacacccgccaccctaggaggtaggtgatgtgaccaccccattgaaagggtagggacgtcgggaaaatatggttgggcacagtggaactagagtttgttccctgtccatccgactccacgagggagaataaaatacgtgtcaagtgctcagaacagcgcctgcggtcaagcactcagtaggtgatatatactgataacataatctgggtggttttaagagcctgcgctccagcccggacacccaccccacccagcccaaggcaccttcctgagcacaggtctgcctgtcccttccccagctcaaaatctttggctctggatggtccaggacactgagcaccaaaatggccttctgtgatctggccctcctgggactctccaaattcattcccccctgctcccctccctgtagatggagaccttccaggcaggtcagacaaactgctgcccccaagtgtagccactgcatctttttcttttttcttttctttctttttttttttctttttcttttttttctctttttttgagacggagtctcactctttgcccaggccggagtggtgcagtggtgtgatcttggctcaccacaacctctacctccggggttcaagcgattctcccgtctcagccccctaagtagctgggattacaggcgccgccaccacgcctggctaattttttgtatttttagtagagacggggtttcaccatgttggcaaggctggtctcaaactcctgacctcaggtgagccgcccgcctcggcctcccaaagccaccgcatcttggtccctgccattcccttagcctggggtgccggctcatcttttccctctaggatttctttagactcagcatatcttgcaaatgtccactaggtggtgctcactcatcgccagcagggagctaacaagccgctcctggggttgggagggcggaggtgccccacagcggggctgacagcctcagcggtcctcttcagcctccagggagccaaccacaggcctgcgtgactctccctgtcatctgcaccctctctggggtcctctgcccatccagccacccgcacagatctgtgtcagtccctgccccccaacactgatcccctcctcccagccctaccccagcctggcactcactggttcttctccagctcaagcaggagctcctggccttcagcctccagggccaccagccccatgtctggcttcgagacctgggcaagaaagagtgtggagctgagatggtggcctccaggcctcctgcctgccagggagtaggtggcctgtggagccggctggggaggaagttcttggggagaacgtgggctggggagtcagcaggaccccccacatactatggagggcgtggaggaggtgagaacatacaaagatgttcccaaactcaggatgtttgcagtcctgacaacagccacttggaagggcgttggcacagcctgccaggcacaccagcatcctccctagagaccagaggtcccagaaaggtgcccctcccctggccccgccctcttctttcatgcccagaaggggcatcaaaagcaggggaagacagaggggtgctgaggacattatgggggcatcgggtagccatggtcagggcctcctcagagcctctgctacctgaggcttggttccaaatgagctgctgctcatttcctatagaattcaaatttgactcctccacttccaattttggcaaactgctccctcttccaaagttttcctgggcctccagcagcccccgtccctccggctccgacacctgcttcactggacccacgaagtaaacatggacgccattccagccaagagagcacactggctctcagctaggtgtcaggaggctggcttggacggccagccctctctccttcccccaccctcttggcgtctcccaccctgtgggaacaccccacttcccccttgtccactcagcctggctgggggcccagagttggagccggcccaggagcttcctgggaggctgctgcgccttcggaatgtttaacccccgactccttttctccaaaaatgcactggcctggggccctgtccaagggtctcagagtctttggagggagttcttccttcgcaagtggggagcagatggtccttgcctccctggccacaggccccacaaggcctccagcatgagctcatgaggctggaatgccacttgctttattggggaaaggtctgcaccgggaaaaaggccatactcgaggtccctgttcctctgcagcccctgctatctttactcttgccctcctggtaccctgccccttgatatatacccctcatcttgaaatgtgagtgtttcctgccttttggaggggatacctagcctctactctttcttctgtaccatcttggcaggcttcctgggggcaggggcccaccggtgggggaagcagagcccctttggggctctcctcttggtcacagcccaggccagacagacagggaggcccagaggcagagtgaccccagtgtgtgtccagccttcccctcctggggatggggagggcaatctcaaagctcaggccagtgccgtgcttgaccagtggaatgggggccttatgggcctaggggatcccagtgagggccctgggttgggagctgctgggtctctgggggcctctcagccttcatggcaatgctcccctgccttccctcttgctggatttggacagtagggctgaaaattccaaacaaagagggctctctaggaggggcaggggtgtagccaatggtttaaaatcgttcagaccttagtgggtctcaggctcccagcctaaagagctgtgtgaccatggacaatttccccaagctctctgggcttccgtttgcccctctgtaaaatgagcatatcaaggctactgccctcttagtttgcagcacagatattatggcacaaacagatggggcatggttattctggaagcgtgtgaagagcgggattgggaagaggctggggcagagcgtcctgcagaagaagcacatggggtggtcttacatctgggggacatcaggagagtgaccactgccccccccataccagaagtggattccacaggagccagtgaggctgaaggttcaggccttcgtggcagggccctgagagggacagcagtgtgtccacagggtcacatgttctggtcaactttgcaaaaggttttctttttggtgcttttttttttttttttttttttagaggctcctgaaaagcttcaggacccacaaactctggacccatttctgcctggtgggggtgggggtggcccagatcatccagggagggagggaaagagggaggtggggtggagaaagctgaaatgacttccatgtgtgcgggctcacgagatccagatgtccaaaccccagtgccttcttctgcccacttgaggggcaggggaggcaggggcctataggagtagtgacttggtggttctggggaccccagcaaaactagaagctgtaatgtagggagagacaaaagggctgggaggttcagggcccctgtggagggcggggagacatggcactgaccggctcctccaggctgacggtgcgccagggttgtccatccaggacccagtgcggggtgactggctgcccagggatatgtcctggagtaaagacagagcacagggtgagggggacctgaggaacacaggggcatgggacaaagcagagggaggggggtagaggacatccccagggaggcactggaggccttttggggcagacttcaccttcaacacgcgtgggctcagcctggagaaggagggacgcccgtgggcatccttggatctgaggagctatcaaggaggaggaaaagagaaggctggaaagggacagctcagctggggacacgggagtcccctgacctttgtcggggggcaggcttgggctcggcgatcacaaggaagaggccaaggccgccagtgcagaggggaaggaaagagcgcggcagccttagggatttttagatgggcagcagatgcctttagggtgagagatgtacgaagagaggacacttgtgccccccccatcatctgagaaaaacaacagccagatgttgccttgcgaggtccaccttgcccagagctccctcggggactctgtcctggtggcagggttttggtaccctggcccagaaggcccctcctcatctcttcaaggggaggggacgcttccggacggagccttggtgctccctggccgggtgtgcctaagggggctctaggaggaatcccagagccaagcattactcagagggcgcctggaatgttcccctggaatgctcccagcccctccactggccccaaccactctcacaggcccgccctgcaggagccaggccccaggcacccagagcctgcagcagccctccttcccccggtacccagtcccagctcccagaacagacagcctcccccctccacgcagccctggcctcagtcctgctgggctgatggctgcctgtggaagtgactcagctcctgctaggccaccccaactccttttttctcctccaccttctctcccagactacaaacatcaaagacccttcctccaagaagccctccttgattggatgagtgaattgccatcaggcagatgagggccgagaggagtctgccaccttggaaaggaggctagaggggccagtgcagggagggctctgagtggatgtgggggaggggaaggaggggaggtctctcagcccagagagcacttaactgagagtagagaaccaagctttgctgctcctaggcctctaagggtttggggaagaggtagggtgggcccgggcacaggtgtggtgtgggtgcagtgtggtgtgtgggtgctgtccacatggccttgcgcgcacgtgctggccacgggcaccctgaccccaatgagggagagaggggcagagctggagctggagctggagctccggtgaccgggtgaatgggggtggaacccgagggagccaggctggtattgggcacatagacgcccctctcccaggggtcccatcacctcccctgaccccaggatagggctcagaggggagggagcagtggaccgcctggggccctcccctggggccagaacagaccaggcccctgtacctgtttggtccccacacagtgctgtggaagccaccgcccagtctgcatagcacagcccagccccgcatgccccctccctggttgccctccctgttcccggccaggcacttgctgtgcaggactggctaatcctcccacccgcttgcagaggttgttccagccccatcttaacatctttgtttggaggggttaccccgaggagacagctgcagtctttccagagcactgctaaacagacaccttctatctggagaggcccttctctatctcaccaaacaaggcaacaatataaacaacatacacactgccctgctgccctgggaggagggacgaggggtgagcagggtggaggccacagctagttctgcagcctgagagcaaagcagggactctgggggactcttgggcatgggggcttcctagaggatggagccccgctgagtcctaaggggtggaggagcaggagcgggtcacacggtggcctgcggatggaagctggttgtgagagcgagaatccaggcagagggggctacggctatgggctgggggctgggggctgggctgtccccgagggggagggagccatgccctctgctttgccagcggagtggcagccgggcagtgtgggcaagtccgggcccggggccagcccaagcacacttgagcgtccctgggcaggtcccacggagacccccccaaagagtccccacgccctgacctactggccgtatggtgccggggccgtgagaccctccgcgcgctgacccgagctctgagcagaacccatccccgccaccaccaccgcgcctagcctgcccctcagggcgcaccccgcccgcgtcctcaccttgaagcaccccggcgcctggcactggccagagcagcagcagtagtagcagcagcagcaacggggtcccccgagctctccggggcctccagcccatagctgtgagctcctcggcctctaggcagcggctcgcaactccggctccgcccaggctggattgcggccgacccgtgcccggtgcagcctcaggccgccgccttcggaccttcccgcccccacctcccaccgcccgccctcgctcccgcctcccctccccgccaaccccgctcggagcctggccaggggccccgacggcgcgcgccatgggggagccgggtcgccactcccggaccgccgcccctcgagggggtggagctgggcggaggagggaatccgtgcggcccctcggatgaccggcccgagccgtccctccccgtcggtctcagagggcctctactcctgagaggaggagagaaccgctgggaaggttcttggaggaccgcggcgtggtgggatgaggcggtgggcaaaggccgcctctcgctgctgaagttggccccaggagcgcgatcttccgtggtctcctggggccgatctctgtcccctccttgctacccgtcctgccccgagggtgccctggcggaggttgagtcgggtcatccacctgcactgggtgcccccaaggataggaaggttcaggcaaccggctgccgctgtcttgggggcttcattgctgggcaaaggcgatgcagcagacggagacaacctttcttccctggcggtggccagagggcagaattgcataaaagctgcagactcccaggcctgggagaccctttcggcctcagtaacatctgtttcatgttttaaacttttgttttcctactcggtgcaaatttggatgagatgttaactttttttttttttttttttttgagatggagtctccctctgtcgccaggctggagtgcagcggcgcgatcttggctcactgcaacctccgactccctggttcaagcgattctcctgcctcagcctcccgagtagctgggactacaggcgcgcgctaccacccccagctaacttttgtatttttagcagagacgaggtttcaccattttggccaggatggtctcaatctcctgatctcgtgatccacccgcctcggcctctcaaagcgctgggattacaggcatgagccaccgcgcccggccggagatgttaacttttaagcaaatctttttttttttttttttttttgagacagagtttctctcttgttacccagactggagtgcaatggcatgatctgggctcactgcaacctctgcctcccagattcaagtgattcttctgcctcagcctcccgagtagctggcattacaggcattcgccaccacgcctggctaattttgtatttttagtagagatggggtttctccatgttggtcaggctggtctcgaactcccgacctcaggtgatctgcccgcctcggcctcccaaagcgctggaattacaggcgtgagacaccgcacccagcctacttttaagtaaatctatttgtttttgagaatttggaatgtagtaatttggttagtgaaagttcgagcagtgagagaaacctacattcacatatctcaaaatcaaaaagtacagaaagcatagggaaaagtctccgtgctcttagccctcctcaccaacaggaaaccaatatgattagtttctttcataggcttttagattattttttcacactcaagacaatacagacatatttttttctcttattaacgtttttctgcactttgattttctttttttttttggtcgcttaatacaccttagatatcagtgcgtttagagggtccttgttgttcttatgattattatttagagacagggtctcactctgtcacccacgctagaggacagtggcctgatcatgcctcattgcagccttgaaatcctgggctcaaggtatcctcccacctcagcctcctgagtagctggaactacaggcacacggcaccaggcccagctaaaatttttaatttttctgtagacagggggtctcactttgtttcccaggctggtctcaaactcctggtcttggccaggcgcagtgtctcatgcctgtaatcccagcactttgggaggccgaggcgggcagatcactggaggtcaggagttcaagaccagtctggccaacatggtgaaaccccatctctactaaaaatacaaaaattagccgggcatggtggtgagcgcctgtagttccagctacttgggaggctgaggcaggaaaatcgcttgaactcagaaggtggaggttgcagcgagccgagatcatgccattgcactccagcctgggcaacaagagcgaaactccgtctcaaaaaataaaaataaaaataaaaagaactcctgatcttaagtgatcctcctgcctcagcttctcaaatcgctggaattacaggagtgagtcaccacagctgtccagctacgagattattacttattattactactttggattttcaaatcaacttcattaaggtataatttacacacaataaaatgcacttattttaagtggccagtaagatgagtttcgataagtgtatataactacataagcatcactataatgcagacacattccctcactcacagaaagagccctgtgcccttccagccaaacttccccactcccaaccccagacagccactgatctgttgttctctgtctatagataagttttgcctgttctagaatttcatataaatggaatcatgcggcatgcactcttctgtgtctggcttccttccctctttccgatgtttttgagattcatttacactattttgcatatcaatagtttgttccttcgtattgctgaatagtgttcggtggtttgagggaaccacagtttctctactcaccagtgcaccatagggttattttccagttaggggctcttataattggaactatatttgcacagagagagagagaggaagaaagagggagagagatatttattatagcaattggctcacgtgattatggaggccaaaaagttcccgaatctgccatctgcaagctggagaacgaggaaagccagtggtgtgattcagtttgagttcaaaggcctgagaaccaggagcaccagtatggaggtggctcgagctcagaacaagttggggacaggaaagcagagcagcaccccagagcagcccctcagcgacacctcttcagtaaagcaaggctgaacacagaggggctggcttcagtgtggatgtcaggtacagaaggcagctcgaggagctactctggcgttcttgcttactggtattcttacctcgaactggccaactcctacttaaactgcaggccatggctttaatgtcctgtcattcagaggctgtcccttacccaaagccaggttagcatcccctgactgacacttctccctgcaacacgtttcagaaggccctgtagtcgtccacttccctgtctctctccccaagctcctgagctccatgtggtctgggaatatgtgtgttgctcacttcctagcacagtcagtgctaataactgactgtagaggggacacagtcgaaaagccacatggggatcagagtcatccttacacagttgacacctcccaaacccagatgagctgtgtccaagtgcaggtcagaggaattttctgccgaagtctctgagaaagggtttatttacattttgaggttgcaggggaggagatgaggccatcaaaccaaagctgaggaagagggatcctaggatgcaccgagcagctccgggggcgcctgacagcacctgggaaagatggcttctccactggcttgttggcgtcaccctccagaggggcatcaggaaatgtcctgggaaccaggcaaaccagtgagcattaacccttagaagtgcttggcatgggtgacacccaccatctgtaaacacgacttctcccaaggagtgacgcagaacaggatgtctgagggaggcactccgactccagccttcagagatcgccagggtggcacctggtgacgacaggctgatgcttgggtgccccagaaaaggtcatgtgtgtgaatgggggccccaaagccaacgcttcatccctgacagcctggtgcatttagaggggaactttttgtcccttggcaaggtgggtggaatttcaggttcatagggcaagggtattttagctttaatagatattgtcaaacagttttccaaagtcattgtacacactctgtgattctacttatgtaaagtttaaaaacaggcaaatcaaatctatggtgttcgaagtcaagacagtagttacccttgtgggggctgcaactggtacagagtgtaaggggggactgtaggatggtctatttcttgatctgggtgtgttcgcttttggaaaagtccttgagttgcatttataatgtgtgaacttttctgtatgttacactttaattgaatgtacaaaaagtctcaggaggcctcagaccactggaagcggacacaactaacccctctgagagcctccaatccaagatggacatatgtccccttggaagtatgcagaagcaggtgaagactcctaagccggatattcccaaatccccccagtagccgcagcttcagcagctgcttatggtcctccctacaccctctcttccccagacagcccccaaacatctggctgcatttgacttgctctctccctgtcccacctctggatttagtccatgttctccaccctccccactgtcagcaatgtagacaagacaaacgcttagttcacgtgcccacctactgcgtgccatgcacggggctggtcattgtgggtggcaaatgtgagcaacacacgaagcctcaaggagcagaaagggacacaaatcacttcagcgtaaggtaatttgtgataaatgtcatgtaacttgcagcccctggccccctcctacagatggtgtctaagaataaaccccactaacatgtgactcctctgttctagcccagctgtttgggttgcaagaaagagactcactccagttgcg

SEQ ID NO: 7 - DNA, 65 bp, Homo sapiens
agtctccgtgctcttagccctcctcaccaacaggaaaccaatatgattagtttctttcataggct

SEQ ID NO: 8 - DNA, 656 bp, Homo sapiens
gtgcagcctcaggccgccgccttcggaccttcccgcccccacctcccaccgcccgccctcgctcccgcctcccctccccgccaaccccgctcggagcctggccaggggccccgacggcgcgcgccatgggggagccgggtcgccactcccggaccgccgcccctcgagggggtggagctgggcggaggagggaatccgtgcggcccctcggatgaccggcccgagccgtccctccccgtcggtctcagagggcctctactcctgagaggaggagagaaccgctgggaaggttcttggaggaccgcggcgtggtgggatgaggcggtgggcaaaggccgcctctcgctgctgaagttggccccaggagcgcgatcttccgtggtctcctggggccgatctctgtcccctccttgctacccgtcctgccccgagggtgccctggcggaggttgagtcgggtcatccacctgcactgggtgcccccaaggataggaaggttcaggcaaccggctgccgctgtcttgggggcttcattgctgggcaaaggcgatgcagcagacggagacaacctttcttccctggcggtggccagagggcagaattgcataaaagctgcagactcccaggcctgggagaccctttcggcctcagtaacat

SEQ ID NO: 9 - DNA, 177 bp, Homo sapiens
cgggcacgggtcggccgcaatccagcctgggcggagccggagttgcgagccgctgcctagaggccgaggagctcacagctatgggctggaggccccggagagctcgggggaccccgttgctgctgctgctactactgctgctgctctggccagtgccaggcgccggggtgcttcaag

SEQ ID NO: 10 - DNA, 80 bp, Homo sapiens
gacatatccctgggcagccagtcaccccgcactgggtcctggatggacaaccctggcgcaccgtcagcctggaggagccg

SEQ ID NO: 11 - DNA, 77 bp, Homo sapiens
gtctcgaagccagacatggggctggtggccctggaggctgaaggccaggagctcctgcttgagctggagaagaacca

SEQ ID NO: 12 - DNA, 79 bp, Homo sapiens
caggctgctggccccaggatacatagaaacccactacggcccagatgggcagccagtggtgctggcccccaaccacacg

SEQ ID NO: 13 - DNA, 119 bp, Homo sapiens
caggctgctggccccaggatacatagaaacccactacggcccagatgggcagccagtggtgctggcccccaaccacacggtgagatgcttccatgggctctgggatgcaccgccagagg

SEQ ID NO: 14 - DNA, 77 bp, Homo sapiens
gatcattgccactaccaagggcgagtaaggggcttccccgactcctgggtagtcctctgcacctgctctgggatgag

SEQ ID NO: 15 - DNA, 190 bp, Homo sapiens
tggcctgatcaccctcagcaggaatgccagctattatctgcgtccctggccaccccggggctccaaggacttctcaacccacgagatctttcggatggagcagctgctcacctggaaaggaacctgtggccacagggatcctgggaacaaagcgggcatgaccagccttcctggtggtccccagagcagg

SEQ ID NO: 16 - DNA, 66 bp, Homo sapiens
ggcaggcgagaagcgcgcaggacccggaagtacctggaactgtacattgtggcagaccacaccctg

SEQ ID NO: 17 - DNA, 72 bp, Homo sapiens
ttcttgactcggcaccgaaacttgaaccacaccaaacagcgtctcctggaagtcgccaactacgtggaccag

SEQ ID NO: 18 - DNA, 167 bp, Homo sapiens
cttctcaggactctggacattcaggtggcgctgaccggcctggaggtgtggaccgagcgggaccgcagccgcgtcacgcaggacgccaacgccacgctctgggccttcctgcagtggcgccgggggctgtgggcgcagcggccccacgactccgcgcagctgctcac

SEQ ID NO: 19 - DNA, 85 bp, Homo sapiens
gggccgcgccttccagggcgccacagtgggcctggcgcccgtcgagggcatgtgccgcgccgagagctcgggaggcgtgagcacg

SEQ ID NO: 20 - DNA, 143 bp, Homo sapiens
gaccactcggagctccccatcggcgccgcagccaccatggcccatgagatcggccacagcctcggcctcagccacgaccccgacggctgctgcgtggaggctgcggccgagtccggaggctgcgtcatggctgcggccaccgg

SEQ ID NO: 21 - DNA, 178 bp, Homo sapiens
gcacccgtttccgcgcgtgttcagcgcctgcagccgccgccagctgcgcgccttcttccgcaaggggggcggcgcttgcctctccaatgccccggaccccggactcccggtgccgccggcgctctgcgggaacggcttcgtggaagcgggcgaggagtgtgactgcggccctggccag

SEQ ID NO: 22 - DNA, 90 bp, Homo sapiens
gagtgccgcgacctctgctgctttgctcacaactgctcgctgcgcccgggggcccagtgcgcccacggggactgctgcgtgcgctgcctg

SEQ ID NO: 23 - DNA, 196 bp, Homo sapiens
ctgaagccggctggagcgctgtgccgccaggccatgggtgactgtgacctccctgagttttgcacgggcacctcctcccactgtcccccagacgtttacctactggacggctcaccctgtgccaggggcagtggctactgctgggatggcgcatgtcccacgctggagcagcagtgccagcagctctgggggcctg

SEQ ID NO: 24 - DNA, 107 bp, Homo sapiens
gctcccacccagctcccgaggcctgtttccaggtggtgaactctgcgggagatgctcatggaaactgcggccaggacagcgagggccacttcctgccctgtgcaggg

SEQ ID NO: 25 - DNA, 199 bp, Homo sapiens
ggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatg

SEQ ID NO: 26 - DNA, 109 bp, Homo sapiens
gtttgcaatagcaaccataactgccactgtgctccaggctgggctccacccttctgtgacaagccaggctttggtggcagcatggacagtggccctgtgcaggctgaaa

SEQ ID NO: 27 - DNA, 148 bp, Homo sapiens
accatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtgg

SEQ ID NO: 28 - DNA, 92 bp, Homo sapiens
ccccaaagatggcccacacagggaccaccccctgggcggcgttcaccccatggagttgggccccacagccactggacagccctggcccctgg

SEQ ID NO: 29 - DNA, 72 bp, Homo sapiens
accctgagaactctcatgagcccagcagccaccctgagaagcctctgccagcagtctcgcctgacccccaag

SEQ ID NO: 30 - DNA, 1031 bp, Homo sapiens
cagatcaagtccagatgccaagatcctgcctctggtgagaggtagctcctaaaatgaacagatttaaagacaggtggccactgacagccactccaggaacttgaactgcaggggcagagccagtgaatcaccggacctccagcacctgcaggcagcttggaagtttcttccccgagtggagcttcgacccacccactccaggaacccagagccacattagaagttcctgagggctggagaacactgctgggcacactctccagctcaataaaccatcagtcccagaagcaaaggtcacacagcccctgacctccctcaccagtggaggctgggtagtgctggccatcccaaaagggctctgtcctgggagtctggtgtgtctcctacatgcaatttccacggacccagctctgtggagggcatgactgctggccagaagctagtggtcctggggccctatggttcgactgagtccacactcccctgcagcctggctggcctctgcaaacaaacataattttggggaccttccttcctgtttcttcccaccctgtcttctcccctaggtggttcctgagcccccacccccaatcccagtgctacacctgaggttctggagctcagaatctgacagcctctcccccattctgtgtgtgtcggggggacagagggaaccatttaagaaaagataccaaagtagaagtcaaaagaaagacatgttggctataggcgtggtggctcatgcctataatcccagcactttgggaagccggggtaggaggatcaccagaggccagcaggtccacaccagcctgggcaacacagcaagacaccgcatctacagaaaaattttaaaattagctgggcgtggtggtgtgtacctgtaggcctagctgctcaggaggctgaagcaggaggatcacttgagcctgagttcaacactgcagtgagctatggtggcaccactgcactccagcctgggtgacagagcaagaccctgtctctaaaataaattttaaaaagacataaaa

SEQ ID NO: 31 - DNA, 78 bp, Homo sapiens
gtgtgccagagcaggcgctgcaggaagaatgccttccaggagcttcagcgctgcctgactgcctgccacagccacggg

SEQ ID NO: 32 - protein, 6 aa, Artificial Sequence
  Description of Artificial Sequence polyhistidine tag
HHHHHH

SEQ ID NO: 33 - protein, 8 aa, Artificial Sequence
  Description of Artificial Sequence FLAG epitope tag
DYKDDDDK

SEQ ID NO: 34 - DNA, 22 bp, Artificial Sequence
  Description of Artificial Sequence Primer
aactcttgaaatgagaagcgtg

SEQ ID NO: 35 - DNA, 22 bp, Artificial Sequence
  Description of Artificial Sequence Primer
aatatcatgcaccatgacccac

SEQ ID NO: 36 - DNA, 22 bp, Artificial Sequence
  Description of Artificial Sequence Primer
tggagtaagtattgtaaactat

SEQ ID NO: 37 - DNA, 22 bp, Artificial Sequence
  Description of Artificial Sequence Primer
ggagcttatcctggattatcta

SEQ ID NO: 38 - DNA, 22 bp, Artificial Sequence
  Description of Artificial Sequence Primer
agagccacacatccatgtcctg

SEQ ID NO: 39 - DNA, 22 bp, Artificial Sequence
  Description of Artificial Sequence Primer
aagccactctgtgaattgccat

SEQ ID NO: 40 - DNA, 22 bp, Artificial Sequence
  Description of Artificial Sequence Primer
gagtagtcgtagtaccagatgg

SEQ ID NO: 41 - DNA, 22 bp, Artificial Sequence
  Description of Artificial Sequence Primer
gtctggcaatggagcatgaaaa

SEQ ID NO: 42 - DNA, 22 bp, Artificial Sequence
  Description of Artificial Sequence Primer
attagagcacatgaaggaaagg

SEQ ID NO: 43 - DNA, 22 bp, Artificial Sequence
  Description of Artificial Sequence Primer
acactgctttgggggacaggct

SEQ ID NO: 44 - DNA, 22 bp, Artificial Sequence
  Description of Artificial Sequence Primer
cacgacgccacagagccagctc

SEQ ID NO: 45 - DNA, 22 bp, Artificial Sequence
  Description of Artificial Sequence Primer
aaccaccacggattcacgcttc

SEQ ID NO: 46 - DNA, 22 bp, Artificial Sequence
  Description of Artificial Sequence Primer
ataaccagatggctgtgggtca

SEQ ID NO: 47 - DNA, 22 bp, Artificial Sequence
  Description of Artificial Sequence Primer
atccccgcaatgaaatagttta

SEQ ID NO: 48 - DNA, 22 bp, Artificial Sequence
  Description of Artificial Sequence Primer
gttgagagcccacttagataat

SEQ ID NO: 49 - DNA, 22 bp, Artificial Sequence
  Description of Artificial Sequence Primer
gcattgggggaagccaggacat

SEQ ID NO: 50 - DNA, 22 bp, Artificial Sequence
  Description of Artificial Sequence Primer
gccactaggaggcaatggcaat

SEQ ID NO: 51 - DNA, 22 bp, Artificial Sequence
  Description of Artificial Sequence Primer
cgacggcatcacggccatctgg

SEQ ID NO: 52 - DNA, 22 bp, Artificial Sequence
  Description of Artificial Sequence Primer
tccaggctcattcattttcatg

SEQ ID NO: 53 - DNA, 22 bp, Artificial Sequence
  Description of Artificial Sequence Primer
tgacatcaacttctcctttcct

SEQ ID NO: 54 - DNA, 22 bp, Artificial Sequence
  Description of Artificial Sequence Primer
agttgcagagacctagcctgtc

SEQ ID NO: 55 - DNA, 22 bp, Artificial Sequence
  Description of Artificial Sequence Primer
tctgggagaggacggagctggc

SEQ ID NO: 56 - DNA, 18 bp, Artificial Sequence
  Description of Artificial Sequence Primer
tgtaggactatattgctc

SEQ ID NO: 57 - DNA, 18 bp, Artificial Sequence
  Description of Artificial Sequence Primer
cgacatttaggtgacact

SEQ ID NO: 58 - DNA, 15 bp, Artificial Sequence
  Description of Artificial Sequence BstXI-linker adapter
gtcttcaccacgggg

SEQ ID NO: 59 - DNA, 11 bp, Artificial Sequence
  Description of Artificial Sequence BstXI-linker adapter
gtggtgaagac

SEQ ID NO: 60 - protein, 9 aa, Artificial Sequence
  Description of Artificial Sequence Synthetic peptide
DPQADQVQM

SEQ ID NO: 61 - protein, 8 aa, Artificial Sequence
  Description of Artificial Sequence Synthetic peptide
DPQDQVQM

SEQ ID NO: 62 - protein, 11 aa, Artificial Sequence
  MOD_RES (1)..(11): “Xaa” represents a variable amino acid
HEXXHXXGXXH

SEQ ID NO: 63 - DNA, 18 bp, Artificial Sequence
  Description of Artificial Sequence Primer
ctgcctagaggccgagga

SEQ ID NO: 64 - DNA, 20 bp, Artificial Sequence
  Description of Artificial Sequence Primer
caggagaccacggaagatcg

SEQ ID NO: 65 - DNA, 20 bp, Artificial Sequence
  Description of Artificial Sequence Primer
ttgcctgaaccttcctatcc

SEQ ID NO: 66 - DNA, 19 bp, Artificial Sequence
  Description of Artificial Sequence Primer
cccctgtgttcctcaggtc

SEQ ID NO: 67 - DNA, 20 bp, Artificial Sequence
  Description of Artificial Sequence Primer
gctccacactctttcttgcc

SEQ ID NO: 68 - DNA, 19 bp, Artificial Sequence
  Description of Artificial Sequence Primer
aggcaggaggaagctgaat

SEQ ID NO: 69 - DNA, 20 bp, Artificial Sequence
  Description of Artificial Sequence Primer
cctaccacaccctccctctt

SEQ ID NO: 70 - DNA, 19 bp, Artificial Sequence
  Description of Artificial Sequence Primer
cctacccctctgcacccta

SEQ ID NO: 71 - DNA, 20 bp, Artificial Sequence
  Description of Artificial Sequence Primer
aacttccttctgggagctgg

SEQ ID NO: 72 - DNA, 20 bp, Artificial Sequence
  Description of Artificial Sequence Primer
cacaccctggtgaggagaga

SEQ ID NO: 73 - DNA, 16 bp, Artificial Sequence
  Description of Artificial Sequence Primer
ccacgaaggaccaccg

SEQ ID NO: 74 - DNA, 18 bp, Artificial Sequence
  Description of Artificial Sequence Primer
ctcacgtgggtgcctctg

SEQ ID NO: 75 - DNA, 18 bp, Artificial Sequence
  Description of Artificial Sequence Primer
ctctacggccgcagtgac

SEQ ID NO: 76 - DNA, 18 bp, Artificial Sequence
  Description of Artificial Sequence Primer
gtccctccatgcccaatg

SEQ ID NO: 77 - DNA, 18 bp, Artificial Sequence
  Description of Artificial Sequence Primer
caggttaagtcggctcgc

SEQ ID NO: 78 - DNA, 19 bp, Artificial Sequence
  Description of Artificial Sequence Primer
ctctctctgccttccccac

SEQ ID NO: 79 - DNA, 20 bp, Artificial Sequence
  Description of Artificial Sequence Primer
tctactgtggggaagatggg

SEQ ID NO: 80 - DNA, 19 bp, Artificial Sequence
  Description of Artificial Sequence Primer
cccctctacttcctcccca

SEQ ID NO: 81 - DNA, 20 bp, Artificial Sequence
  Description of Artificial Sequence Primer
gaccttggggttcctaatcc

SEQ ID NO: 82 - DNA, 19 bp, Artificial Sequence
  Description of Artificial Sequence Primer
gtgcacctgctcaggactc

SEQ ID NO: 83 - DNA, 21 bp, Artificial Sequence
  Description of Artificial Sequence Primer
cctggactcttatcacgttgc

SEQ ID NO: 84 - DNA, 20 bp, Artificial Sequence
  Description of Artificial Sequence Primer
ttaccctccaccatttctcc

SEQ ID NO: 85 - DNA, 20 bp, Artificial Sequence
  Description of Artificial Sequence Primer
gtggagagggaagggagaag

SEQ ID NO: 86 - DNA, 20 bp, Artificial Sequence
  Description of Artificial Sequence Primer
ccccatgggttgaatttaca

SEQ ID NO: 87 - DNA, 21 bp, Artificial Sequence
  Description of Artificial Sequence Primer
gcagctaggcctacaggtaca

SEQ ID NO: 88 - DNA, 20 bp, Artificial Sequence
  Description of Artificial Sequence Primer
accacgcctatagccaacat

SEQ ID NO: 89 - DNA, 20 bp, Artificial Sequence
  Description of Artificial Sequence Primer
aggtgtagcactgggattgg

SEQ ID NO: 90 - DNA, 20 bp, Artificial Sequence
  Description of Artificial Sequence Primer
ccccaggaccactagcttct

SEQ ID NO: 91 - DNA, 20 bp, Artificial Sequence
  Description of Artificial Sequence Primer
attgagctggagagtgtgcc

SEQ ID NO: 92 - DNA, 20 bp, Artificial Sequence
  Description of Artificial Sequence Primer
ttcaagttcctggagtggct

SEQ ID NO: 93 - DNA, 20 bp, Artificial Sequence
  Description of Artificial Sequence Primer
acaaggaccctctaaacgca

SEQ ID NO: 94 - DNA, 20 bp, Artificial Sequence
  Description of Artificial Sequence Primer
acccttctgtgacaagccag

SEQ ID NO: 95 - DNA, 20 bp, Artificial Sequence
  Description of Artificial Sequence Primer
gtgttgctaccgactcccag

SEQ ID NO: 96 - DNA, 18 bp, Artificial Sequence
  Description of Artificial Sequence Primer
cccaggtgcagagagcag

SEQ ID NO: 97 - DNA, 21 bp, Artificial Sequence
  Description of Artificial Sequence Primer
gctcctcttgtccactctcct

SEQ ID NO: 98 - DNA, 20 bp, Artificial Sequence
  Description of Artificial Sequence Primer
gccacttcctctgcacaaat

SEQ ID NO: 99 - DNA, 20 bp, Artificial Sequence
  Description of Artificial Sequence Primer
ttctctgtgacctgggtggt

SEQ ID NO: 100 - DNA, 18 bp, Artificial Sequence
  Description of Artificial Sequence Primer
atttgggccagagatggg

SEQ ID NO: 101 - DNA, 18 bp, Artificial Sequence
  Description of Artificial Sequence Primer
ggcagaggagcaaggtgg

SEQ ID NO: 102 - DNA, 20 bp, Artificial Sequence
  Description of Artificial Sequence Primer
atggcttggaatcatcaagg

SEQ ID NO: 103 - DNA, 20 bp, Artificial Sequence
  Description of Artificial Sequence Primer
tagagagaggaggtgccagc

SEQ ID NO: 104 - DNA, 18 bp, Artificial Sequence
  Description of Artificial Sequence Primer
aaagatggcccacacagg

SEQ ID NO: 105 - DNA, 20 bp, Artificial Sequence
  Description of Artificial Sequence Primer
agaactctcatgagcccagc

SEQ ID NO: 106 - DNA, 20 bp, Artificial Sequence
  Description of Artificial Sequence Primer
agctctgagcagaacccatc

SEQ ID NO: 107 - DNA, 18 bp, Artificial Sequence
  Description of Artificial Sequence Primer
ctcgagggggtggagctg

SEQ ID NO: 108 - DNA, 20 bp, Artificial Sequence
  Description of Artificial Sequence Primer
gagaggaggagagaaccgct

SEQ ID NO: 109 - DNA, 20 bp, Artificial Sequence
  Description of Artificial Sequence Primer
agtgacttggtggttctggg

SEQ ID NO: 110 - DNA, 20 bp, Artificial Sequence
  Description of Artificial Sequence Primer
tgtcatctgcaccctctctg

SEQ ID NO: 111 - DNA, 20 bp, Artificial Sequence
  Description of Artificial Sequence Primer
aagagggagggtgtggtagg

SEQ ID NO: 112 - DNA, 20 bp, Artificial Sequence
  Description of Artificial Sequence Primer
gtgatcaggccactagggtg

SEQ ID NO: 113 - DNA, 20 bp, Artificial Sequence
  Description of Artificial Sequence Primer
atacagcattcccactccca

SEQ ID NO: 114 - DNA, 18 bp, Artificial Sequence
  Description of Artificial Sequence Primer
gaaggcagaaatcccggt

SEQ ID NO: 115 - DNA, 18 bp, Artificial Sequence
  Description of Artificial Sequence Primer
caccagcacctgcctgtc

SEQ ID NO: 116 - DNA, 17 bp, Artificial Sequence
  Description of Artificial Sequence Primer
gggtcagaggcacccac

SEQ ID NO: 117 - DNA, 19 bp, Artificial Sequence
  Description of Artificial Sequence Primer
gccgtagagcctcctgtct

SEQ ID NO: 118 - DNA, 19 bp, Artificial Sequence
  Description of Artificial Sequence Primer
gacgaccaaagaaacgcag

SEQ ID NO: 119 - DNA, 18 bp, Artificial Sequence
  Description of Artificial Sequence Primer
tgagcggagagggcaagt

SEQ ID NO: 120 - DNA, 20 bp, Artificial Sequence
  Description of Artificial Sequence Primer
aaaccctcaccctgaacctt

SEQ ID NO: 121 - DNA, 19 bp, Artificial Sequence
  Description of Artificial Sequence Primer
aagggtgctcgtgtcctct

SEQ ID NO: 122 - DNA, 20 bp, Artificial Sequence
  Description of Artificial Sequence Primer
ccactcagctccactcccta

SEQ ID NO: 123 - DNA, 19 bp, Artificial Sequence
  Description of Artificial Sequence Primer
ggattcaaacggcaaggag

SEQ ID NO: 124 - DNA, 19 bp, Artificial Sequence
  Description of Artificial Sequence Primer
gctgagtcctgagcaggtg

SEQ ID NO: 125 - DNA, 19 bp, Artificial Sequence
  Description of Artificial Sequence Primer
gaaccgcaggagtaggctc

SEQ ID NO: 126 - DNA, 20 bp, Artificial Sequence
  Description of Artificial Sequence Primer
atatggtcagcaggagaccc

SEQ ID NO: 127 - DNA, 21 bp, Artificial Sequence
  Description of Artificial Sequence Primer
gcatcctggtctccatgataa

SEQ ID NO: 128 - DNA, 20 bp, Artificial Sequence
  Description of Artificial Sequence Primer
gaggctttgaatccaggtcc

SEQ ID NO: 129 - DNA, 20 bp, Artificial Sequence
  Description of Artificial Sequence Primer
cagcaagacaccgcatctac

SEQ ID NO: 130 - DNA, 20 bp, Artificial Sequence
  Description of Artificial Sequence Primer
gggacagagggaaccattta

SEQ ID NO: 131 - DNA, 20 bp, Artificial Sequence
  Description of Artificial Sequence Primer
ttccttcctgtttcttccca

SEQ ID NO: 132 - DNA, 20 bp, Artificial Sequence
  Description of Artificial Sequence Primer
gtcctgggagtctggtgtgt

SEQ ID NO: 133 - DNA, 20 bp, Artificial Sequence
  Description of Artificial Sequence Primer
aggaacccagagccacacta

SEQ ID NO: 134 - DNA, 20 bp, Artificial Sequence
  Description of Artificial Sequence Primer
tgcctctggtgagaggtagc

SEQ ID NO: 135 - DNA, 20 bp, Artificial Sequence
  Description of Artificial Sequence Primer
ttcctggatcactggtcctc

SEQ ID NO: 136 - DNA, 21 bp, Artificial Sequence
  Description of Artificial Sequence Primer
ttcgagcagtgagagaaacct

SEQ ID NO: 137 - DNA, 19 bp, Artificial Sequence
  Description of Artificial Sequence Primer
ctgggagtcggtagcaaca

SEQ ID NO: 138 - DNA, 18 bp, Artificial Sequence
  Description of Artificial Sequence Primer
aggccactggaacctcct

SEQ ID NO: 139 - DNA, 20 bp, Artificial Sequence
  Description of Artificial Sequence Primer
gcagcatggtacagggactg

SEQ ID NO: 140 - DNA, 20 bp, Artificial Sequence
  Description of Artificial Sequence Primer
cagctgaccagtggtatgga

SEQ ID NO: 141 - DNA, 20 bp, Artificial Sequence
  Description of Artificial Sequence Primer
tgtcagacatggccacagag

SEQ ID NO: 142 - DNA, 20 bp, Artificial Sequence
  Description of Artificial Sequence Primer
agggtcctcttagctgccac

SEQ ID NO: 143 - DNA, 20 bp, Artificial Sequence
  Description of Artificial Sequence Primer
aggccttgtcatttcctgtg

SEQ ID NO: 144 - DNA, 20 bp, Artificial Sequence
  Description of Artificial Sequence Primer
caaagaaccttggatgtccg

SEQ ID NO: 145 - DNA, 19 bp, Artificial Sequence
  Description of Artificial Sequence Primer
ctcagctcccttcctgctc

SEQ ID NO: 146 - DNA, 18 bp, Artificial Sequence
  Description of Artificial Sequence Primer
ctgtgtgggccatctttg

SEQ ID NO: 147 - DNA, 20 bp, Artificial Sequence
  Description of Artificial Sequence Primer
ggagaaatggtggagggtaa

SEQ ID NO: 148 - DNA, 19 bp, Artificial Sequence
  Description of Artificial Sequence Primer
aaagccacagcttctccct

SEQ ID NO: 149 - DNA, 20 bp, Artificial Sequence
  Description of Artificial Sequence Primer
aggtttctgggctcaggtta

SEQ ID NO: 150 - DNA, 19 bp, Artificial Sequence
  Description of Artificial Sequence Primer
gtaggtgtgccagagcagg

SEQ ID NO: 151 - DNA, 21 bp, Artificial Sequence
  Description of Artificial Sequence Primer
tgtggacctagaatggtgagc

SEQ ID NO: 152 - DNA, 20 bp, Artificial Sequence
  Description of Artificial Sequence Primer
caaagtcacacaacaagcgg

SEQ ID NO: 153 - DNA, 20 bp, Artificial Sequence
  Description of Artificial Sequence Primer
caggatcttggcatctggac

SEQ ID NO: 154 - DNA, 20 bp, Artificial Sequence
  Description of Artificial Sequence Primer
ctggcttgtcacagaagggt

SEQ ID NO: 155 - DNA, 20 bp, Artificial Sequence
  Description of Artificial Sequence Primer
ctggagcacagtggcagtta

SEQ ID NO: 156 - DNA, 20 bp, Artificial Sequence
  Description of Artificial Sequence Primer
tttggtcgtccctcagtttc

SEQ ID NO: 157 - DNA, 20 bp, Artificial Sequence
  Description of Artificial Sequence Primer
cctctcaggagtagaggccc

SEQ ID NO: 158 - DNA, 20 bp, Artificial Sequence
  Description of Artificial Sequence Primer
agcggttctctcctcctctc

SEQ ID NO: 159 - DNA, 20 bp, Artificial Sequence
  Description of Artificial Sequence Primer
cctctcaggagtagaggccc

SEQ ID NO: 160 - DNA, 20 bp, Artificial Sequence
  Description of Artificial Sequence Primer
atgttactgaggccgaaagg

SEQ ID NO: 161 - DNA, 20 bp, Artificial Sequence
  Description of Artificial Sequence Primer
ccctttccagccttctcttt

SEQ ID NO: 162 - DNA, 20 bp, Artificial Sequence
  Description of Artificial Sequence Primer
caggactgcaaacatcctga

SEQ ID NO: 163 - DNA, 18 bp, Artificial Sequence
  Description of Artificial Sequence Primer
tccctggtgcttcccata

SEQ ID NO: 164 - DNA, 19 bp, Artificial Sequence
  Description of Artificial Sequence Primer
aggcaggaggaagctgaat

SEQ ID NO: 165 - DNA, 18 bp, Artificial Sequence
  Description of Artificial Sequence Primer
cctcttgcccctcttgct

SEQ ID NO: 166 - DNA, 21 bp, Artificial Sequence
  Description of Artificial Sequence Primer
cctgaatgtccagagtcctga

SEQ ID NO: 167 - DNA, 20 bp, Artificial Sequence
  Description of Artificial Sequence Primer
ggcctcgagtcccagtattt

SEQ ID NO: 168 - DNA, 20 bp, Artificial Sequence
  Description of Artificial Sequence Primer
agagcctcctgtctctccct

SEQ ID NO: 169 - DNA, 18 bp, Artificial Sequence
  Description of Artificial Sequence Primer
tcgccctcagcttctcag

SEQ ID NO: 170 - DNA, 18 bp, Artificial Sequence
  Description of Artificial Sequence Primer
tcacgtgggtgcctctga

SEQ ID NO: 171 - DNA, 20 bp, Artificial Sequence
  Description of Artificial Sequence Primer
gggttacttcccctctctgg

SEQ ID NO: 172 - DNA, 18 bp, Artificial Sequence
  Description of Artificial Sequence Primer
ctgggctttccaccctgg

SEQ ID NO: 173 - DNA, 18 bp, Artificial Sequence
  Description of Artificial Sequence Primer
ctgggctttccaccctgg

SEQ ID NO: 174 - DNA, 19 bp, Artificial Sequence
  Description of Artificial Sequence Primer
tccaggtggtgaactctgc

SEQ ID NO: 175 - DNA, 20 bp, Artificial Sequence
  Description of Artificial Sequence Primer
tagaatggtgagctctgccc

SEQ ID NO: 176 - DNA, 20 bp, Artificial Sequence
  Description of Artificial Sequence Primer
gaccttggggttcctaatcc

SEQ ID NO: 177 - DNA, 19 bp, Artificial Sequence
  Description of Artificial Sequence Primer
ccaagcacacttgagcgtc

SEQ ID NO: 178 - DNA, 18 bp, Artificial Sequence
  Description of Artificial Sequence Primer
agccatgccctctgcttt

SEQ ID NO: 179 - DNA, 18 bp, Artificial Sequence
  Description of Artificial Sequence Primer
cagcccaagcacacttga

SEQ ID NO: 180 - DNA, 20 bp, Artificial Sequence
  Description of Artificial Sequence Primer
cccatagctgtgagctcctc

SEQ ID NO: 181 - DNA, 20 bp, Artificial Sequence
  Description of Artificial Sequence Primer
aaagcttcaggacccacaaa

SEQ ID NO: 182 - DNA, 19 bp, Artificial Sequence
  Description of Artificial Sequence Primer
atcttggtccctgccattc

SEQ ID NO: 183 - DNA, 18 bp, Artificial Sequence
  Description of Artificial Sequence Primer
gagggagctctttcccca

SEQ ID NO: 184 - DNA, 18 bp, Artificial Sequence
  Description of Artificial Sequence Primer
ggaccaccaggaaggctg

SEQ ID NO: 185 - DNA, 18 bp, Artificial Sequence
  Description of Artificial Sequence Primer
aaccccagctcccagaag

SEQ ID NO: 186 - DNA, 20 bp, Artificial Sequence
  Description of Artificial Sequence Primer
ctgctcacctggaaaggaac

SEQ ID NO: 187 - DNA, 19 bp, Artificial Sequence
  Description of Artificial Sequence Primer
actgcaggaaggcccagag

SEQ ID NO: 188 - DNA, 20 bp, Artificial Sequence
  Description of Artificial Sequence Primer
accgaaacttgaaccacacc

SEQ ID NO: 189 - DNA, 20 bp, Artificial Sequence
  Description of Artificial Sequence Primer
tgagggacgaccaaagaaac

SEQ ID NO: 190 - DNA, 20 bp, Artificial Sequence
  Description of Artificial Sequence Primer
caaagtcacacaacaagcgg

SEQ ID NO: 191 - DNA, 20 bp, Artificial Sequence
  Description of Artificial Sequence Primer
gaacctgagggcaccaatta

SEQ ID NO: 192 - DNA, 21 bp, Artificial Sequence
  Description of Artificial Sequence Primer
ttggccttagttaattggtgc

SEQ ID NO: 193 - DNA, 21 bp, Artificial Sequence
  Description of Artificial Sequence Primer
ttggccttagttaattggtgc

SEQ ID NO: 194 - DNA, 20 bp, Artificial Sequence
  Description of Artificial Sequence Primer
ctggagcacagtggcagtta

SEQ ID NO: 195 - DNA, 20 bp, Artificial Sequence
  Description of Artificial Sequence Primer
aggagtaggctcaggaagca

SEQ ID NO: 196 - DNA, 20 bp, Artificial Sequence
  Description of Artificial Sequence Primer
tgtactgggaggtagagggc

SEQ ID NO: 197 - DNA, 20 bp, Artificial Sequence
  Description of Artificial Sequence Primer
agagggtgacttggagcaga

SEQ ID NO: 198 - DNA, 20 bp, Artificial Sequence
  Description of Artificial Sequence Primer
aggcaataacccactcagga

SEQ ID NO: 199 - DNA, 21 bp, Artificial Sequence
  Description of Artificial Sequence Primer
cccatgggttgaatttacata

SEQ ID NO: 200 - DNA, 20 bp, Artificial Sequence
  Description of Artificial Sequence Primer
gcctctggtgatcctcctac

SEQ ID NO: 201 - DNA, 20 bp, Artificial Sequence
  Description of Artificial Sequence Primer
actcagtcgaaccatagggc

SEQ ID NO: 202 - DNA, 20 bp, Artificial Sequence
  Description of Artificial Sequence Primer
tgtgtgacctttgcttctgg

SEQ ID NO: 203 - DNA, 20 bp, Artificial Sequence
  Description of Artificial Sequence Primer
gcatgaagcaatgggagaat

SEQ ID NO: 204 - DNA, 20 bp, Artificial Sequence
  Description of Artificial Sequence Primer
actcagtcgaaccatagggc

SEQ ID NO: 205 - DNA, 20 bp, Artificial Sequence
  Description of Artificial Sequence Primer
gcaggaaggtgtcatggtct

SEQ ID NO: 206 - DNA, 20 bp, Artificial Sequence
  Description of Artificial Sequence Primer
gcaggaaggtgtcatggtct

SEQ ID NO: 207 - DNA, 18 bp, Artificial Sequence
  Description of Artificial Sequence Primer
gggcattggagaggcaag

SEQ ID NO: 208 - DNA, 20 bp, Artificial Sequence
  Description of Artificial Sequence Primer
tctgcctcccagattcaagt

SEQ ID NO: 209 - DNA, 20 bp, Artificial Sequence
  Description of Artificial Sequence Primer
agaatgccttccaggagctt

SEQ ID NO: 210 - DNA, 20 bp, Artificial Sequence
  Description of Artificial Sequence Primer
gtgttgctaccgactcccag

SEQ ID NO: 211 - DNA, 20 bp, Artificial Sequence
  Description of Artificial Sequence Primer
ctgcttcctgagcctactcc

SEQ ID NO: 212 - DNA, 19 bp, Artificial Sequence
  Description of Artificial Sequence Primer
aacaggaggttccagtggc

SEQ ID NO: 213 - DNA, 20 bp, Artificial Sequence
  Description of Artificial Sequence Primer
agcgagttgtgattgagggt

SEQ ID NO: 214 - DNA, 20 bp, Artificial Sequence
  Description of Artificial Sequence Primer
tgtgcaggctgaaagtatgc

SEQ ID NO: 215 - DNA, 20 bp, Artificial Sequence
  Description of Artificial Sequence Primer
gccacttcctctgcacaaat

SEQ ID NO: 216 - DNA, 20 bp, Artificial Sequence
  Description of Artificial Sequence Primer
ctgagcccagaaacctgatt

SEQ ID NO: 217 - DNA, 18 bp, Artificial Sequence
  Description of Artificial Sequence Primer
gtgagtgaggcaccaggg

SEQ ID NO: 218 - DNA, 20 bp, Artificial Sequence
  Description of Artificial Sequence Primer
cctagatggccaggaagtga

SEQ ID NO: 219 - DNA, 20 bp, Artificial Sequence
  Description of Artificial Sequence Primer
ccagaaacctgattaggggg

SEQ ID NO: 220 - DNA, 20 bp, Artificial Sequence
  Description of Artificial Sequence Primer
tacctctcaccagaggcagg

SEQ ID NO: 221 - DNA, 20 bp, Artificial Sequence
  Description of Artificial Sequence Primer
gccagaagctagtggtcctg

SEQ ID NO: 222 - DNA, 19 bp, Artificial Sequence
  Description of Artificial Sequence Primer
gcaggcagcttggaagttt

SEQ ID NO: 223 - DNA, 21 bp, Artificial Sequence
  Description of Artificial Sequence Primer
ttatcatggagaccaggatgc

SEQ ID NO: 224 - DNA, 20 bp, Artificial Sequence
  Description of Artificial Sequence Primer
gacctggattcaaagcctcc

SEQ ID NO: 225 - DNA, 20 bp, Artificial Sequence
  Description of Artificial Sequence Primer
atgttggctataggcgtggt

SEQ ID NO: 226 - DNA, 21 bp, Artificial Sequence
  Description of Artificial Sequence Primer
ttatcatggagaccaggatgc

SEQ ID NO: 227 - DNA, 20 bp, Artificial Sequence
  Description of Artificial Sequence Primer
ctgagtggagggagcagaag

SEQ ID NO: 228 - DNA, 20 bp, Artificial Sequence
  Description of Artificial Sequence Primer
ctgagtggagggagcagaag

SEQ ID NO: 229 - DNA, 18 bp, Artificial Sequence
  Description of Artificial Sequence Primer
ccatgagatcggccacag

SEQ ID NO: 230 - DNA, 20 bp, Artificial Sequence
  Description of Artificial Sequence Primer
atttcaaggctgcaatgagg

SEQ ID NO: 231 - DNA, 20 bp, Artificial Sequence
  Description of Artificial Sequence Primer
acttctttccatggcctctg

SEQ ID NO: 232 - DNA, 20 bp, Artificial Sequence
  Description of Artificial Sequence Primer
accacccaggtcacagagaa

SEQ ID NO: 233 - DNA, 20 bp, Artificial Sequence
  Description of Artificial Sequence Primer
tcccaagaccaggctatgtc

SEQ ID NO: 234 - DNA, 18 bp, Artificial Sequence
  Description of Artificial Sequence Primer
ctggggatgagaagcagc

SEQ ID NO: 235 - DNA, 20 bp, Artificial Sequence
  Description of Artificial Sequence Primer
cttctcccttccctctccac

SEQ ID NO: 236 - DNA, 20 bp, Artificial Sequence
  Description of Artificial Sequence Primer
atttgtgcagaggaagtggc

SEQ ID NO: 237 - DNA, 20 bp, Artificial Sequence
  Description of Artificial Sequence Primer
catttcctccaggctctgac

SEQ ID NO: 238 - DNA, 20 bp, Artificial Sequence
  Description of Artificial Sequence Primer
tcagagcctggaggaaatgt

SEQ ID NO: 239 - DNA, 19 bp, Artificial Sequence
  Description of Artificial Sequence Primer
gttcctggagtgggtgggt

SEQ ID NO: 240 - DNA, 19 bp, Artificial Sequence
  Description of Artificial Sequence Primer
ctgggagtcggtagcaaca

SEQ ID NO: 241 - DNA, 41 bp, Homo sapiens
gccctctgagaccgacggggagggacggctcgggccggtca

SEQ ID NO: 242 - DNA, 41 bp, Homo sapiens
caagaaccttcccagcggttctctcctcctctcaggagtag

SEQ ID NO: 243 - DNA, 41 bp, Homo sapiens
caccatctcagctccacactctttcttgcccaggtctcgaa

SEQ ID NO: 244 - DNA, 41 bp, Homo sapiens
ccaccatctcagctccacactctttcttgcccaggtctcga

SEQ ID NO: 245 - DNA, 41 bp, Homo sapiens
acaactaagccatcaccaaggctccttcctctagccccaag

SEQ ID NO: 246 - DNA, 41 bp, Homo sapiens
tggtgcttcccatattcacatctcccacaactaagccatca

SEQ ID NO: 247 - DNA, 41 bp, Homo sapiens
caggatacatagaaacccactacggcccagatgggcagcca

SEQ ID NO: 248 - DNA, 41 bp, Homo sapiens
ccctccaaatcagaagagacaggaattcacaggcctcgagt

SEQ ID NO: 249 - DNA, 41 bp, Homo sapiens
agctgctcacctggaaaggaacctgtggccacagggatcct

SEQ ID NO: 250 - DNA, 41 bp, Homo sapiens
acttccttctgggagctggggttgggggtcagggctcaagc

SEQ ID NO: 251 - DNA, 41 bp, Homo sapiens
ttcctgcagtggcgccgggggctgtgggcgcagcggcccca

SEQ ID NO: 252 - DNA, 41 bp, Homo sapiens
ggttcagggtgagggtttcggggagcttgggagccggcctg

SEQ ID NO: 253 - DNA, 41 bp, Homo sapiens
cagagaagcgcgggggttgggggactgtccctccatgccca

SEQ ID NO: 254 - DNA, 41 bp, Homo sapiens
cccctctctgggctctgcgcgtctggcggctgtagccaagc

SEQ ID NO: 255 - DNA, 41 bp, Homo sapiens
cagccgccgccagctgcgcgccttcttccgcaaggggggcg

SEQ ID NO: 256 - DNA, 41 bp, Homo sapiens
agtggcctcccagtcaagcgagggggtggatccctgcccca

SEQ ID NO: 257 - DNA, 41 bp, Homo sapiens
tgctggccatgctcctcagcgtcctgctgcctctgctccca

SEQ ID NO: 258 - DNA, 41 bp, Homo sapiens
ctgctgcctctgctcccaggggccggcctggcctggtgttg

SEQ ID NO: 259 - DNA, 41 bp, Homo sapiens
gaagtagctttgaacaggaggttccagtggcctcccagtca

SEQ ID NO: 260 - DNA, 41 bp, Homo sapiens
gcctctgtctcaccagttttcggccctttgccacttcctct

SEQ ID NO: 261 - DNA, 41 bp, Homo sapiens
acaaatcacctctgtcacccccttgaagttcccaaatgctg

SEQ ID NO: 262 - DNA, 41 bp, Homo sapiens
tccataccactggtcagctgcggtgctggctgcccctgtgc

SEQ ID NO: 263 - DNA, 41 bp, Homo sapiens
ggtgctggctgcccctgtgccagggccctgccttaacccag

SEQ ID NO: 264 - DNA, 41 bp, Homo sapiens
ggaaatgacaaggccttgggggatgggatggggacagtcaa

SEQ ID NO: 265 - DNA, 41 bp, Homo sapiens
agggctcatgcctcctgcctccttccagatgggcagcaccc

SEQ ID NO: 266 - DNA, 41 bp, Homo sapiens
gcccctccccagccccagggtctcctgctgaccatattcac

SEQ ID NO: 267 - DNA, 41 bp, Homo sapiens
cctgggcggcgttcaccccatggagttgggccccacagcca

SEQ ID NO: 268 - DNA, 41 bp, Homo sapiens
gccccacagccactggacagccctggcccctgggtgagtga

SEQ ID NO: 269 - DNA, 41 bp, Homo sapiens
gccctggcccctgggtgagtgaggcaccagggggaggtgga

SEQ ID NO: 270 - DNA, 41 bp, Homo sapiens
tgcagcctggggccccagtccttaggggacaacatatcctc

SEQ ID NO: 271 - DNA, 41 bp, Homo sapiens
cactgagtgaggatgggctctctgccacacagcttgcagcc

SEQ ID NO: 272 - DNA, 41 bp, Homo sapiens
ctggtcctcactgagtgaggatgggctctctgccacacagc

SEQ ID NO: 273 - DNA, 41 bp, Homo sapiens
atgacctcttggttatcatggagaccaggatgctggaagcc

SEQ ID NO: 274 - DNA, 41 bp, Homo sapiens
agcaagacaccgcatctacagaaaaattttaaaattagctg

SEQ ID NO: 275 - DNA, 41 bp, Homo sapiens
ggaggatcaccagaggccagcaggtccacaccagcctgggc

SEQ ID NO: 276 - DNA, 41 bp, Homo sapiens
atcccagcactttgggaagccggggtaggaggatcaccaga

SEQ ID NO: 277 - DNA, 41 bp, Homo sapiens
agcctggctggcctctgcaaacaaacataattttggggacc

SEQ ID NO: 278 - DNA, 41 bp, Homo sapiens
actgagtccacactcccctgcagcctggctggcctctgcaa

SEQ ID NO: 279 - DNA, 41 bp, Homo sapiens
tccaggaacccagagccacattagaagttcctgagggctgg

SEQ ID NO: 280 - DNA, 41 bp, Homo sapiens
ttcttccccgagtggagcttcgacccacccactccaggaac

SEQ ID NO: 281 - DNA, 41 bp, Homo sapiens
tcctcattctcagcagatcaagtccagatgccaagatcctg

SEQ ID NO: 282 - DNA, 41 bp, Homo sapiens
ctgaggaccacacggggtggtggttggcggggtggtggttg

SEQ ID NO: 283 - DNA, 41 bp, Homo sapiens
ggctggcaggccgagcctagatggcagccagagccccaggc

SEQ ID NO: 284 - DNA, 41 bp, Homo sapiens
ctttgctctgtcactcctgcctcccttgggcgttcacattc

SEQ ID NO: 285 - DNA, 41 bp, Homo sapiens
gtgagctctgcccacccgacccctccttgccgtttgaatcc

SEQ ID NO: 286 - DNA, 41 bp, Homo sapiens
tggcgaggttactcctacaccgggaggagcaccgtcgggtc

SEQ ID NO: 287 - DNA, 41 bp, Homo sapiens
ggctgctcactattggggccgcatcgtcccctgtcccgctt

SEQ ID NO: 288 - DNA, 41 bp, Homo sapiens
gccgcatcgtcccctgtcccgcttgttgtgtgactttgcgc

SEQ ID NO: 289 - DNA, 17 bp, Artificial Sequence
  Description of Artificial Sequence Primer
gccgtcccaccccgtcg

SEQ ID NO: 290 - DNA, 17 bp, Artificial Sequence
  Description of Artificial Sequence Primer
cctcctctcttggcgac

SEQ ID NO: 291 - DNA, 18 bp, Artificial Sequence
  Description of Artificial Sequence Primer
tccacactctttcttgcc

SEQ ID NO: 292 - DNA, 20 bp, Artificial Sequence
  Description of Artificial Sequence Primer
gctccacactctttcttgcc

SEQ ID NO: 293 - DNA, 18 bp, Artificial Sequence
  Description of Artificial Sequence Primer
tcaccaaggctccttcct

SEQ ID NO: 294 - DNA, 21 bp, Artificial Sequence
  Description of Artificial Sequence Primer
cagaagagacaggaattcaca

SEQ ID NO: 295 - DNA, 19 bp, Artificial Sequence
  Description of Artificial Sequence Primer
tggaaaggaacctgtggcc

SEQ ID NO: 296 - DNA, 17 bp, Artificial Sequence
  Description of Artificial Sequence Primer
gggtttcggggagcttg

SEQ ID NO: 297 - DNA, 16 bp, Artificial Sequence
  Description of Artificial Sequence Primer
gggttgggggactgtc

SEQ ID NO: 298 - DNA, 16 bp, Artificial Sequence
  Description of Artificial Sequence Primer
ctctgcgcgtctggcg

SEQ ID NO: 299 - DNA, 17 bp, Artificial Sequence
  Description of Artificial Sequence Primer
gccgtccctccccgtcg

SEQ ID NO: 300 - DNA, 20 bp, Artificial Sequence
  Description of Artificial Sequence Primer
tcctcctctattggcgaccc

SEQ ID NO: 301 - DNA, 21 bp, Artificial Sequence
  Description of Artificial Sequence Primer
ctccacactttttcttgccca

SEQ ID NO: 302 - DNA, 19 bp, Artificial Sequence
  Description of Artificial Sequence Primer
gctccacactctttcttgc

SEQ ID NO: 303 - DNA, 18 bp, Artificial Sequence
  Description of Artificial Sequence Primer
tcaccaagcctccttcct

SEQ ID NO: 304 - DNA, 19 bp, Artificial Sequence
  Description of Artificial Sequence Primer
agaagagacgggaattcac

SEQ ID NO: 305 - DNA, 17 bp, Artificial Sequence
  Description of Artificial Sequence Primer
tggaaaggagcctgtgg

SEQ ID NO: 306 - DNA, 19 bp, Artificial Sequence
  Description of Artificial Sequence Primer
agggtttcgtggagcttgg

SEQ ID NO: 307 - DNA, 18 bp, Artificial Sequence
  Description of Artificial Sequence Primer
ggggttggaggactgtcc

SEQ ID NO: 308 - DNA, 18 bp, Artificial Sequence
  Description of Artificial Sequence Primer
gctctgcgcatctggcgg

SEQ ID NO: 309 - DNA, 18 bp, Artificial Sequence
  Description of Artificial Sequence Primer
agtcaagcgagggggtgg

SEQ ID NO: 310 - DNA, 16 bp, Artificial Sequence
  Description of Artificial Sequence Primer
cctcagcgtcctgctg

SEQ ID NO: 311 - DNA, 18 bp, Artificial Sequence
  Description of Artificial Sequence Primer
aacaggaggttccagtgg

SEQ ID NO: 312 - DNA, 18 bp, Artificial Sequence
  Description of Artificial Sequence Primer
accagttttcggcccttt

SEQ ID NO: 313 - DNA, 18 bp, Artificial Sequence
  Description of Artificial Sequence Primer
ctgtcacccccttgaagt

SEQ ID NO: 314 - DNA, 16 bp, Artificial Sequence
  Description of Artificial Sequence Primer
tcagctgcggtgctgg

SEQ ID NO: 315 - DNA, 15 bp, Artificial Sequence
  Description of Artificial Sequence Primer
gccttgggggatgga

SEQ ID NO: 316 - DNA, 16 bp, Artificial Sequence
  Description of Artificial Sequence Primer
tcctgcctccttccag

SEQ ID NO: 317 - DNA, 16 bp, Artificial Sequence
  Description of Artificial Sequence Primer
actggacagccctggc

SEQ ID NO: 318 - DNA, 19 bp, Artificial Sequence
  Description of Artificial Sequence Primer
ctgtgtggcagagagccca

SEQ ID NO: 319 - DNA, 22 bp, Artificial Sequence
  Description of Artificial Sequence Primer
aattatgtttgtttgcagaggc

SEQ ID NO: 320 - DNA, 19 bp, Artificial Sequence
  Description of Artificial Sequence Primer
gaacttctagtgtggctct

SEQ ID NO: 321 - DNA, 17 bp, Artificial Sequence
  Description of Artificial Sequence Primer
ccaagggaggcaggagt

SEQ ID NO: 322 - DNA, 18 bp, Artificial Sequence
  Description of Artificial Sequence Primer
agtcaagcgtgggggtgg

SEQ ID NO: 323 - DNA, 19 bp, Artificial Sequence
  Description of Artificial Sequence Primer
ctcctcagcatcctgctgc

SEQ ID NO: 324 - DNA, 20 bp, Artificial Sequence
  Description of Artificial Sequence Primer
gaacaggagtttccagtggc

SEQ ID NO: 325 - DNA, 20 bp, Artificial Sequence
  Description of Artificial Sequence Primer
caccagtttttggccctttg

SEQ ID NO: 326 - DNA, 20 bp, Artificial Sequence
  Description of Artificial Sequence Primer
ctgtcacccacttgaagttc

SEQ ID NO: 327 - DNA, 18 bp, Artificial Sequence
  Description of Artificial Sequence Primer
ggtcagctgtggtgctgg

SEQ ID NO: 328 - DNA, 19 bp, Artificial Sequence
  Description of Artificial Sequence Primer
aggccttgggagatgggat

SEQ ID NO: 329 - DNA, 16 bp, Artificial Sequence
  Description of Artificial Sequence Primer
tcctgccttcttccag

SEQ ID NO: 330 - DNA, 16 bp, Artificial Sequence
  Description of Artificial Sequence Primer
actggacagtcctggc

SEQ ID NO: 331 - DNA, 16 bp, Artificial Sequence
  Description of Artificial Sequence Primer
tgtggcagggagccca

SEQ ID NO: 332 - DNA, 20 bp, Artificial Sequence
  Description of Artificial Sequence Primer
attatgtttgcttgcagagg

SEQ ID NO: 333 - DNA, 21 bp, Artificial Sequence
  Description of Artificial Sequence Primer
ggaacttctaatgtggctctg

SEQ ID NO: 334 - DNA, 20 bp, Artificial Sequence
  Description of Artificial Sequence Primer
cccaagggaagcaggagtga

SEQ ID NO: 335 - protein, 55 aa, Homo sapiens
CCFAHNCSLRPGAQCAHGDCCVRCLLKPAGALCRQAMGDCDLPEFCTGTSSHCPP

SEQ ID NO: 336 - protein, 11 aa, Homo sapiens
TMAHEIGHSLG

SEQ ID NO: 337 - protein, 86 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHR

SEQ ID NO: 338 - protein, 48 aa, Homo sapiens
MGWRPRRARGTPLLLLLLLLLLWPVPGAGVLQGHIPGQPVTPHWVLDG

SEQ ID NO: 339 - protein, 178 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHRLLAPGYIETHYGPDGQPVVLAPNHTVRCFHGLWDAPPEDHCHYQGRVRGFPDSWVVLCTCSGMSGLITLSRNASYYLRPWPPRGSKDFSTHE

SEQ ID NO: 340 - protein, 113 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHGLITLSRNASYYLRPWPPRGSKDFSTHE

SEQ ID NO: 341 - protein, 165 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHRLLAPGYIETHYGPDGQPVVLAPNHTDHCHYQGRVRGFPDSWVVLCTCSGMSGLITLSRNASYYLRPWPPRGSKDFSTHE

SEQ ID NO: 342 - protein, 168 aa, Homo sapiens
LAPGYIETHYGPDGQPVVLAPNHTDHCHYQGRVRGFPDSWVVLCTCSGMSGLITLSRNASYYLRPWPPRGSKDFSTHEIFRMEQLLTWKGTCGHRDPGNKAGMTSLPGGPQSRGRRKARRTRKYLELYIVADHTLFLTRHRNLNHTKQRLLEVANYVDQLLRTLDIQV

SEQ ID NO: 343 - protein, 167 aa, Homo sapiens
SGYCWDGACPTLEQQCQQLWGPGSHPAPEACFQVVNSAGDAHGNCGQDSEGHFLPCAGRDALCGKLQCQGGKPSLLAPHMVPVDSTVHLDGQEVTCRGALALPSAQLDLLGLGLVEPGTQCGPRMVCNSNHNCHCAPGWAPPFCDKPGFGGSMDSGPVQAENHDTFL

SEQ ID NO: 344 - protein, 193 aa, Homo sapiens
SGYCWDGACPTLEQQCQQLWGPGSHPAPEACFQVVNSAGDAHGNCGQDSEGHFLPCAGRDALCGKLQCQGGKPSLLAPHMVPVDSTVHLDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFL

SEQ ID NO: 345 - protein, 126 aa, Homo sapiens
SGYCWDGACPTLEQQCQQLWGPDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFL

SEQ ID NO: 346 - protein, 93 aa, Homo sapiens
AWCCYRLPGAHLQRCSWGCRRDPACSGPKDGPHRDHPLGGVHPMELGPTATGQPWPLDPENSHEPSSHPEKPLPAVSPDPQADQVQMPRSCLW

SEQ ID NO: 347 - protein, 236 aa, Homo sapiens
SGYCWDGACPTLEQQCQQLWGPDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFLLAMLLSVLLPLLPGAGLAWCCYRLPGAHLQRCSWGCRRDPACSGPKDGPHRDHPLGGVHPMELGPTATGQPWPLDPENSHEPSSHPEKPLPAVSPDPQADQVQMPRSCLW

SEQ ID NO: 348 - protein, 302 aa, Homo sapiens
SGYCWDGACPTLEQQCQQLWGPGSHPAPEACFQVVNSAGDAHGNCGQDSEGHFLPCAGRDALCGKLQCQGGKPSLLAPHMVPVDSTVHLDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFLLAMLLSVLLPLLPGAGLAWCCYRLPGAHLQRCSWGCRRDPACSGPKDGPHRDHPLGGVHPMELGPTATGQPWPLDPENSHEPSSHPEKPLPAVSPDPQDQVQMPRSCLW

SEQ ID NO: 349 - protein, 235 aa, Homo sapiens
SGYCWDGACPTLEQQCQQLWGPDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFLLAMLLSVLLPLLPGAGLAWCCYRLPGAHLQRCSWGCRRDPACSGPKDGPHRDHPLGGVHPMELGPTATGQPWPLDPENSHEPSSHPEKPLPAVSPDPQDQVQMPRSCLW

SEQ ID NO: 350 - DNA, 339 bp, Homo sapiens
cgggcacgggtcggccgcaatccagcctgggcggagccggagttgcgagccgctgcctagaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccacaggc

SEQ ID NO: 351 - DNA, 225 bp, Homo sapiens
cgggcacgggtcggccgcaatccagcctgggcggagccggagttgcgagccgctgcctagaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggac

SEQ ID NO: 352 - DNA, 562 bp, Homo sapiens
gcctagaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccacaggctgctggccccaggatacatagaaacccactacggcccagatgggcagccagtggtgctggcccccaaccacacggtgagatgcttccatgggctctgggatgcaccgccagaggatcattgccactaccaagggcgagtaaggggcttccccgactcctgggtagtcctctgcacctgctctgggatgagtggcctgatcaccctcagcaggaatgccagctattatctgcgtccctggccaccccggggctccaaggacttctcaacccacgagat

SEQ ID NO: 353 - DNA, 362 bp, Homo sapiens
gaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccatggcctgatcaccctcagcaggaatgccagctattatctgcgtccctggccaccccggggctccaaggacttctcaacccacgagat

SEQ ID NO: 354 - DNA, 518 bp, Homo sapiens
gaggccgaggagctcacagctatgggctggaggccccggagagctcgggggaccccgttgctgctgctgctactactgctgctgctctggccagtgccaggcgccggggtgcttcaaggacatatccctgggcagccagtcaccccgcactgggtcctggatggacaaccctggcgcaccgtcagcctggaggagccggtctcgaagccagacatggggctggtggccctggaggctgaaggccaggagctcctgcttgagctggagaagaaccacaggctgctggccccaggatacatagaaacccactacggcccagatgggcagccagtggtgctggcccccaaccacacggatcattgccactaccaagggcgagtaaggggcttccccgactcctgggtagtcctctgcacctgctctgggatgagtggcctgatcaccctcagcaggaatgccagctattatctgcgtccctggccaccccggggctccaaggacttctcaacccacgagat

SEQ ID NO: 355 - DNA, 506 bp, Homo sapiens
ctggccccaggatacatagaaacccactacggcccagatgggcagccagtggtgctggcccccaaccacacggatcattgccactaccaagggcgagtaaggggcttccccgactcctgggtagtcctctgcacctgctctgggatgagtggcctgatcaccctcagcaggaatgccagctattatctgcgtccctggccaccccggggctccaaggacttctcaacccacgagatctttcggatggagcagctgctcacctggaaaggaacctgtggccacagggatcctgggaacaaagcgggcatgaccagccttcctggtggtccccagagcaggggcaggcgaaaagcgcgcaggacccggaagtacctggaactgtacattgtggcagaccacaccctgttcttgactcggcaccgaaacttgaaccacaccaaacagcgtctcctggaagtcgccaactacgtggaccagcttctcaggactctggacattcaggtggc

SEQ ID NO: 356 - DNA, 503 bp, Homo sapiens
cagtggctactgctgggatggcgcatgtcccacgctggagcagcagtgccagcagctctgggggcctggctcccacccagctcccgaggcctgtttccaggtggtgaactctgcgggagatgctcatggaaactgcggccaggacagcgagggccacttcctgccctgtgcagggagggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatggtttgcaatagcaaccataactgccactgtgctccaggctgggctccacccttctgtgacaagccaggctttggtggcagcatggacagtggccctgtgcaggctgaaaaccatgacaccttcctgc

SEQ ID NO: 357 - DNA, 581 bp, Homo sapiens
cagtggctactgctgggatggcgcatgtcccacgctggagcagcagtgccagcagctctgggggcctggctcccacccagctcccgaggcctgtttccaggtggtgaactctgcgggagatgctcatggaaactgcggccaggacagcgagggccacttcctgccctgtgcagggagggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgc

SEQ ID NO: 358 - DNA, 380 bp, Homo sapiens
cagtggctactgctgggatggcgcatgtcccacgctggagcagcagtgccagcagctctgggggcctgatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgc

SEQ ID NO: 359 - DNA, 324 bp, Homo sapiens
ggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagcagatcaagtccagatgccaagatcctgcctctggtgagaggtagctcctaaaatgaacagatttaaagacaggtggcc

SEQ ID NO: 360 - DNA, 753 bp, Homo sapiens
cagtggctactgctgggatggcgcatgtcccacgctggagcagcagtgccagcagctctgggggcctgatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagcagatcaagtccagatgccaagatcctgcctctggtgagaggtagctcctaaaatgaacagatttaaagacaggtggcc

SEQ ID NO: 361 - DNA, 1154 bp, Homo sapiens
cagtggctactgctgggatggcgcatgtcccacgctggagcagcagtgccagcagctctgggggcctggctcccacccagctcccgaggcctgtttccaggtggtgaactctgcgggagatgctcatggaaactgcggccaggacagcgagggccacttcctgccctgtgcagggagggatgccctgtgtgggaagctgcagtgccagggtggaaagcccagcctgctcgcaccgcacatggtgccagtggactctaccgttcacctagatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagatcaagtccagatgccaagatcctgcctctggtgagaggtagctcctaaaatgaacagatttaaagacaggtggccactgacagccactccaggaacttgaactgcaggggcagagccagtgaatcaccggacctccagcacctgcaggcagcttggaagtttcttccccgagtggagcttcgacccacccactccaggaacccagagccacattagaagttcctgagggctggagaacactgctgggcacactctccagctcaataaaccatcagtcc

SEQ ID NO: 362 - DNA, 953 bp, Homo sapiens
cagtggctactgctgggatggcgcatgtcccacgctggagcagcagtgccagcagctctgggggcctgatggccaggaagtgacttgtcggggagccttggcactccccagtgcccagctggacctgcttggcctgggcctggtagagccaggcacccagtgtggacctagaatggtgtgccagagcaggcgctgcaggaagaatgccttccaggagcttcagcgctgcctgactgcctgccacagccacggggtttgcaatagcaaccataactgccactgtgctccaggctgggctccacccttctgtgacaagccaggctttggtggcagcatggacagtggccctgtgcaggctgaaaaccatgacaccttcctgctggccatgctcctcagcgtcctgctgcctctgctcccaggggccggcctggcctggtgttgctaccgactcccaggagcccatctgcagcgatgcagctggggctgcagaagggaccctgcgtgcagtggccccaaagatggcccacacagggaccaccccctgggcggcgttcaccccatggagttgggccccacagccactggacagccctggcccctggaccctgagaactctcatgagcccagcagccaccctgagaagcctctgccagcagtctcgcctgacccccaagatcaagtccagatgccaagatcctgcctctggtgagaggtagctcctaaaatgaacagatttaaagacaggtggccactgacagccactccaggaacttgaactgcaggggcagagccagtgaatcaccggacctccagcacctgcaggcagcttggaagtttcttccccgagtggagcttcgacccacccactccaggaacccagagccacattagaagttcctgagggctggagaacactgctgggcacactctccagctcaataaaccatcagtcc

SEQ ID NO: 363 - protein, 812 aa, Homo sapiens
MGWRPRRARGTPLLLLLLLLLLWPVPGAGVLQGHIPGQPVTPHWVLDGQPWRTVSLEEPVSKPDMGLVALEAEGQELLLELEKNHRLLAPGYIETHYGPDGQPVVLAPNHTDHCHYQGRVRGFPDSWVVLCTCSGMSGLITLSRNASYYLRPWPPRGSKDFSTHEIFRMEQLLTWKGTCGHRDPGNKAGMTSLPGGPQSRGRREARRTRKYLELYIVADHTLFLTRHRNLNHTKQRLLEVANYVDQLLRTLDIQVALTGLEVWTERDRSRVTQDANATLWAFLQWRRGLWAQRPHDSAQLLTGRAFQGATVGLAPVEGMCRAESSGGVSTDHSELPIGAAATMAHEIGHSLGLSHDPDGCCVEAAAESGGCVMAAATGHPFPRVFSACSRRQLRAFFRKGGGACLSNAPDPGLPVPPALCGNGFVEAGEECDCGPGQECRDLCCFAHNCSLRPGAQCAHGDCCVRCLLKPAGALCRQAMGDCDLPEFCTGTSSHCPPDVYLLDGSPCARGSGYCWDGACPTLEQQCQQLWGPGSHPAPEACFQVVNSAGDAHGNCGQDSEGHFLPCAGRDALCGKLQCQGGKPSLLAPHMVPVDSTVHLDGQEVTCRGALALPSAQLDLLGLGLVEPGTQCGPRMVCQSRRCRKNAFQELQRCLTACHSHGVCNSNHNCHCAPGWAPPFCDKPGFGGSMDSGPVQAENHDTFLLAMLLSVLLPLLPGAGLAWCCYRLPGAHLQRCSWGCRRDPACSGPKDGPHRDHPLGGVHPMELGPTATGQPWPLDPENSHEPSSHPEKPLPAVSPDPQDQVQMPRSCLW

(19) United States
(12) Patent Application Publication
(10) Pub. No.: US 2003/0138925 A1
(43) Pub. Date: Jul. 24, 2003

(54) Novel human gene relating to respiratory diseases, obesity, and inflammatory bowel disease

(76) Inventors: Tim Keith, Bedford, MA (US); RANDALL LITTLE, NEWTONVILLE, MA (US); PAUL VAN EERDEWEGH, WESTON, MA (US); JOSEE DUPUIS, NEWTON, MA (US); RICHARD DEL MASTRO, NORFOLK, MA (US); JASON SIMON, WESTFIELD, NJ (US); KRISTINA ALLEN, HOPKINTON, MA (US); SUNIL PANDIT, GAITHERSBURG, MD (US)

Correspondence Address: MORGAN & FINNEGAN, L.L.P., 345 Park Avenue, New York, NY, 10154-0053, US

(21) Appl. No.: 09/834,597

(22) Filed: Apr. 13, 2001

Related U.S. Application Data
(63) Continuation-in-part of application No. 09/548,797, filed on Apr. 13, 2000.

(51) Int. Cl. C12N 9/00; C07H 21/04; C12N 1/18; C12N 1/21; C12P 21/02; C12N 5/06

(52) U.S. Cl. 435/183; 435/69.1; 435/320.1; 435/325; 435/252.3; 435/254.2; 536/23.2

(57) ABSTRACT

This invention relates to genes identified from human chromosome 20p13-p12, which are associated with various diseases, including asthma. The invention also relates to the nucleotide sequences of these genes, isolated nucleic acids comprising these nucleotide sequences, and isolated polypeptides or peptides encoded thereby. The invention further relates to vectors and host cells comprising the disclosed nucleotide sequences, or fragments thereof, as well as antibodies that bind to the encoded polypeptides or peptides. Also related are ligands that modulate the activity of the disclosed genes or gene products. In addition, the invention relates to methods and compositions employing the disclosed nucleic acids, polypeptides or peptides, antibodies, and/or ligands for use in diagnostics and therapeutics for asthma and other diseases.

### RELATED APPLICATIONS

This application is a continuation-in-part of U.S. application Ser. No. 09/548,797, filed Apr. 13, 2000, which is incorporated by reference in its entirety.

### FIELD OF THE INVENTION

This invention relates to genes identified from human chromosome 20p13-p12, including Gene 216, which are associated with asthma, obesity, inflammatory bowel disease, and other human diseases. The invention also relates to the nucleotide sequences of these genes, including genomic DNA sequences, cDNA sequences, and single nucleotide polymorphisms. The invention further relates to isolated nucleic acids comprising these nucleotide sequences, and isolated polypeptides or peptides encoded thereby. Also related are expression vectors and host cells comprising the disclosed nucleic acids or fragments thereof, as well as antibodies that bind to the encoded polypeptides or peptides. The present invention further relates to ligands that modulate the activity of the disclosed genes or gene products. In addition, the invention relates to diagnostics and therapeutics for various diseases, including asthma, utilizing the disclosed nucleic acids, polypeptides or peptides, antibodies, and/or ligands.

### BACKGROUND

Mouse chromosome 2 has been linked to a variety of disorders including airway hyperesponsiveness and obesity (DeSanctis et al., 1995, Nature Genetics, 11:150-154; Nagle et al., 1999, Nature, 398:148-152). This region of the mouse genome is homologous to portions of human chromosome 20 including 20p13-p12. Although human chromosome 20p13-12p has been linked to a variety of genetic disorders including diabetes insipidus, neurohypophyseal, congenital endothelial dystrophy of cornea, insomnia, neurodegeneration with brain iron accumulation 1 (Hallervorden-Spatz syndrome), fibrodysplasia ossificans progressive, alagille syndrome, hydrometrocolpos (McKusick-Kaufman syndrome), Creutzfeldt-Jakob disease and Gerstmann-Straussler disease (see NCBI; National Center for Biotechnology Information, National Library of Medicine, 38A, 8N905, 8600 Rockville Pike, Bethesda, Md. 20894; www.ncbi.nim.nih.gov) the genes affecting these disorders have yet to be discovered. There is a need in the art for identifying specific genes relating to these disorders, as well as genes associated with obesity, lung disease, particularly, inflammatory lung disease phenotypes such as Chronic Obstructive Lung Disease (COPD), Adult Respiratory Distress Syndrome (ARDS), and asthma. Identification and characterization of such genes will make possible the development of effective diagnostics and therapeutic means to treat lung-related disorders.

### SUMMARY OF THE INVENTION

This invention relates to Gene 216 located on human chromosome 20p13-p12. In specific embodiments, the invention relates to isolated nucleic acids comprising Gene 216 genomic sequences (e.g., SEQ ID NO:5 and SEQ ID NO:6), cDNA sequences (e.g., SEQ ID NO:1 and SEQ ID NO:3), complementary sequences, sequence variants, or fragments thereof, as described herein. The present invention also encompasses nucleic acid probes or primers useful for assaying a biological sample for the presence or expression of Gene 216. The invention further encompasses nucleic acids variants comprising single nucleotide polymorphisms (SNPs) identified in several genes, including Gene 216 (e.g., SEQ ID NO:241-288). Such SNPs can be used to diagnose diseases such as asthma, or to determine a genetic predisposition thereto. In addition, the present invention encompasses nucleic acids comprising alternate splicing variants (e.g., SEQ ID NO:2 and SEQ ID NO:350-362).

This invention also relates to vectors and host cells comprising vectors comprising the Gene 216 nucleic acid sequences disclosed herein. Such vectors can be used for nucleic acid preparations, including antisense nucleic acids, and for the expression of encoded polypeptides or peptides. Host cells can be prokaryotic or eukaryotic cells. In specific embodiments, an expression vector comprises a DNA sequence encoding the Gene 216 polypeptide sequence (e.g., SEQ ID NO:4 or SEQ ID NO:363), sequence variants, or fragments thereof, as described herein.

The present invention further relates to isolated Gene 216 polypeptides and peptides. In specific embodiments, the polypeptides or peptides comprise the amino acid sequence of the Gene 216 (e.g., SEQ ID NO:4 or SEQ ID NO:363), sequence variants, or portions thereof, as described herein. In addition, this invention encompasses isolated fusion proteins comprising Gene 216 polypeptides or peptides.

The present invention also relates to isolated antibodies, including monoclonal and polyclonal antibodies, and antibody fragments, that are specifically reactive with the Gene 216 polypeptides, fusion proteins, or variants, or portions thereof, as disclosed herein. In specific embodiments, monoclonal antibodies are prepared to be specifically reactive with the Gene 216 polypeptide (e.g., SEQ ID NO:4 or SEQ ID NO:363) or peptides, or sequence variants thereof.

In addition, the present invention relates to methods of obtaining Gene 216 polynucleotides and polypeptides, variant sequences, or fragments thereof, as disclosed herein. Also related are methods of obtaining anti-Gene 216 antibodies and antibody fragments. The present invention also encompasses methods of obtaining Gene 216 ligands, e.g., agonists, antagonists, inhibitors, and binding factors. Such ligands can be used as therapeutics for asthma and related diseases.

The present invention also relates to diagnostic methods and kits utilizing Gene 216 (wild-type, mutant, or variant) nucleic acids, polypeptides, antibodies, or functional fragments thereof. Such factors can be used, for example, in diagnostic methods and kits for measuring expression levels of Gene 216, and to screen for various Gene 216-related diseases, especially asthma. In addition, the nucleic acids described herein can be used to identify chromosomal abnormalities affecting Gene 216, and to identify allelic variants or mutations of Gene 216 in an individual or population.

The present invention further relates to methods and therapeutics for the treatment of various diseases, including asthma. In various embodiments, therapeutics comprising the disclosed Gene 216 nucleic acids, polypeptides, antibodies, ligands, or variants, derivatives, or portions thereof, are administered to a subject to treat, prevent, or ameliorate asthma. Specifically related are therapeutics comprising Gene 216 antisense nucleic acids, monoclonal antibodies, metalloprotease inhibitors, and gene therapy vectors. Such therapeutics can be administered alone, or in combination with one or more asthma treatments.

In addition, this invention relates to non-human transgenic animals and cell lines comprising one or more of the disclosed Gene 216 nucleic acids, which can be used for drug screening, protein production, and other purposes. Also related are non-human knock-out animals and cell lines, wherein one or more endogenous Gene 216 genes (i.e., orthologs), or portions thereof, are deleted or replaced by marker genes.

This invention further relates to methods of identifying proteins that are candidates for being involved in asthma (i.e., a “candidate protein”). Such proteins are identified by a method comprising: 1) identifying a protein in a first individual having the asthma phenotype; 2) identifying a protein in a second individual not having the asthma phenotype; and 3) comparing the protein of the first individual to the protein of the second individual, wherein a) the protein that is present in the second individual but not the first individual is the candidate protein; or b) the protein that is present in a higher amount in the second individual than in the first individual is the candidate protein; or c) the protein that is present in a lower amount in the second individual than in the first individual is the candidate protein.

### BRIEF DESCRIPTION OF THE FIGURES

FIG. 1 depicts the LOD Plot of Linkage to Asthma.

FIG. 2 depicts the LOD Plot of Linkage to BHR(PC20<=4 mg/ml) & Asthma.

FIG. 3 depicts the LOD Plot of Linkage to BHR(PC20<=16 mg/ml) & Asthma

FIG. 4 depicts the LOD Plot of Linkage to High Total IgE & Asthma

FIG. 5 depicts the LOD Plot of Linkage to High Specific IgE & Asthma

FIG. 6 depicts the BAC/STS content contig map of human chromosome 20p13-p12.

FIG. 7 depicts the BAC1098L22 nucleotide sequence (SEQ ID NO:5).

FIG. 8 depicts the locations of single nucleotide polymorphisms, corresponding amino acid changes, and domains in the Gene 216 transcript. The exons of the transcript are marked from A to T and the size of each one is indicated. Above the exons, the 8 domains are labeled and a black bar represents the approximate location of each one. Underneath the black bars are the approximate location of the amino acid changes that have been identified. The amino acids boxed in white are the alleles that are most frequently observed. The nucleotides boxed in gray are the alleles that are most frequently observed. Single nucleotide polymorphisms are unboxed, and the polymorphism names appear underneath. The uterus cDNA clone does not contain all of Exon A, and does not contain the sequence CAG between Exon S and T.

FIG. 9 depicts alternate splice variants of Gene 216 obtained from lung tissue, including rt672 (SEQ ID NO:350), rt690 (SEQ ID NO:351), rt709 (SEQ ID NO:352), rt711 (SEQ ID NO:353), rt713 (SEQ ID NO:354), and rt720 (SEQ ID NO:355).

FIG. 10 depicts alternate splice variants of Gene 216 obtained from lung tissue, including rt725 (SEQ ID NO:356), rt727 (SEQ ID NO:357), rt733 (SEQ ID NO:358), rt735 (SEQ ID NO:359), rt764 (SEQ ID NO:360), rt772 (SEQ ID NO:361), and rt774 (SEQ ID NO:362).

FIG. 11 depicts the structure of the genomic sequence of Gene 216.

FIG. 12 depicts the alternate AG splice sequences at the junction of Intron ST and Exon T in Gene 216.

FIG. 13 depicts the promoter region of Gene 216. The Gene 216 promoter sequence is shown in SEQ ID NO:8; the Gene 216 enhancer sequence is shown in SEQ ID NO:7.

FIG. 14 depicts a dendrogram of the ADAM family members and the relationship of Gene 216 to ADAMs that possesses an active metalloprotease domain.

FIGS. 15A-15C depict Northern Blots illustrating Gene 216 expression patterns. FIGS. 15A-15B show Gene 216 expression in various tissue types. FIG. 15C shows Gene 216 expression in bronchial smooth muscle tissue.

FIG. 16 depicts a Dot Blot that shows Gene 216 expression in various tissue types.

FIG. 17 depicts RT-PCR analysis of Gene 216 expression in primary cells from lung tissue.

FIG. 18 depicts an amino acid sequence alignment (Pileup) of 5 ADAM family members that are closely related to Gene 216. Amino acids highlighted in black show 100% identity within the Pileup; dark gray show 80% identity; and light gray show 60% identity. The boxed amino acids represent the cysteine switch, the metalloprotease domain, and the “met-turn”. The labeled arrows show the locations of the 8 domains.

FIG. 19 depicts the amino acid sequence of Gene 216 (SEQ ID NO:4). Labeled arrows above the sequence denote domain and corresponding length. Black boxes represent the signal sequence and the transmembrane domain identified by hydrophobicity plots. The underlined cysteine residue at position 133 is predicted to be involved in the cysteine switch, the dashed box represents the metalloprotease domain, and the methionine underlined twice is the “met-turn”. The gray boxes represent the signaling binding sites identified in the cytoplasmic tail. The amino acid changes corresponding to single nucleotide polymorphisms are indicated in bold. The alanine deleted in the uterus cDNA clone is marked within a black triangle, and if present would have been between the glutamine and the aspartic acid.

FIG. 20 depicts the Kyte-Doolittle hydrophobicity plot for the Gene 216 amino acid sequence.

FIGS. 21 depicts the genomic sequence of the mouse ortholog of Gene 216 (SEQ ID NO:364).

FIG. 22 depicts the cDNA nucleotide sequence (SEQ ID NO:364) and predicted amino acid sequence (SEQ ID NO:365) of the mouse ortholog of Gene 216.

FIG. 23 depicts an amino acid sequence alignment (Pileup) of human Gene 216 polypeptide (SEQ ID NO:4) and the mouse ortholog of Gene 216 (SEQ ID NO:366). Vertical lines indicate identical amino acid residues. Dots indicate similar amino acid residues.

FIG. 24 depicts the nucleotide sequence (SEQ ID NO:1) and encoded amino acid sequence (SEQ ID NO:4) determined from the master cDNA sequence of Gene 216. The master cDNA sequence combines the sequence information from the uterine cDNA clone and 5′ RACE clone. Identified single nucleotide polymorphism positions are underlined.

FIG. 25 depicts the results of a case control study p-value plot that shows single nucleotide polymorphism association with the asthma phenotype in the combined US and UK populations.

FIG. 26 depicts the results of a case control study p-value plot that shows single nucleotide polymorphism association with the asthma phenotype in the US and UK populations, separately.

FIG. 27 depicts the results of a case control study p-value plot that shows single nucleotide polymorphism association with the bronchial hyper-responsiveness and asthma phenotypes in the US and UK combined population.

FIG. 28 depicts the results of a case control study p-value plot that shows single nucleotide polymorphism association with the bronchial hyper-responsiveness and asthma phenotypes in the US and UK populations, separately.

FIG. 29 depicts the genomic nucleotide sequence (SEQ ID NO:6) determined for Gene 216. Identified single nucleotide polymorphism positions are underlined.

FIG. 30 depicts the nucleotide sequence (SEQ ID NO:3) and encoded amino acid sequence (SEQ ID NO: 363) of Gene 216 determined from the uterus cDNA clone. Identified single nucleotide polymorphism positions are underlined.

FIG. 31 depicts the nucleotide sequence (SEQ ID NO:350) and encoded amino acid sequence (SEQ ID NO:337) of Gene 216 alternate splice variant rt672.

FIG. 32 depicts the nucleotide sequence (SEQ ID NO:351) and encoded amino acid sequence (SEQ ID NO:338) of Gene 216 alternate splice variant rt690.

FIG. 33 depicts the nucleotide sequence (SEQ ID NO:352) and encoded amino acid sequence (SEQ ID NO:339) of Gene 216 alternate splice variant rt709.

FIG. 34 depicts the nucleotide sequence (SEQ ID NO:353) and encoded amino acid sequence (SEQ ID NO:340) of Gene 216 alternate splice variant rt711.

FIG. 35 depicts the nucleotide sequence (SEQ ID NO:354) and encoded amino acid sequence (SEQ ID NO:341) of Gene 216 alternate splice variant rt713.

FIG. 36 depicts the nucleotide sequence (SEQ ID NO:355) and encoded amino acid sequence (SEQ ID NO:342) of Gene 216 alternate splice variant rt720.

FIG. 37 depicts the nucleotide sequence (SEQ ID NO:356) and encoded amino acid sequence (SEQ ID NO:343) of Gene 216 alternate splice variant rt725.

FIG. 38 depicts the nucleotide sequence (SEQ ID NO:357) and encoded amino acid sequence (SEQ ID NO:344) of Gene 216 alternate splice variant rt727.

FIG. 39 depicts the nucleotide sequence (SEQ ID NO:358) and encoded amino acid sequence (SEQ ID NO:345) of Gene 216 alternate splice variant rt733.

FIG. 40 depicts the nucleotide sequence (SEQ ID NO:359) and encoded amino acid sequence (SEQ ID NO:346) of Gene 216 alternate splice variant rt735.

FIG. 41 depicts the nucleotide sequence (SEQ ID NO:360) and encoded amino acid sequence (SEQ ID NO:347) of Gene 216 alternate splice variant rt764.

FIG. 42 depicts the nucleotide sequence (SEQ ID NO:361) and encoded amino acid sequence (SEQ ID NO:348) of Gene 216 alternate splice variant rt772.

FIG. 43 depicts the nucleotide sequence (SEQ ID NO:362) and encoded amino acid sequence (SEQ ID NO:349) of Gene 216 alternate splice variant rt774.

### DETAILED DESCRIPTION OF THE INVENTION

Gene 216 was identified by extensive analysis of the region of human chromosome 20p13-p12 associated with airway hyperresponsiveness, asthma, and atopy. This region has also been implicated in other diseases such as obesity (Wilson, 1999, Arch. Intern. Med. 159:2513-4). Bronchial asthma, furthermore, has been linked to intestinal conditions such as inflammatory bowel disease (B. Wallaert et al., 1995, J. Exp. Med. 182:1897-1904). Thus, there was a need to identify and isolate the gene(s) associated with this region of human chromosome 20.

Definitions

To aid in the understanding of the specification and claims, the following definitions are provided.

“Disorder region” refers to a portion of the human chromosome 20 bounded by the markers D20S502 and D20S851. A “disorder-associated” nucleic acid or polypeptide sequence refers to a nucleic acid sequence that maps to region 20p13-p12 or the polypeptides encoded therein (e.g., Gene 216 nucleic acids, and polypeptides). For nucleic acids, this encompasses sequences that are identical or complementary to the Gene 216 sequence, as well as sequence-conservative, function-conservative, and non-conservative variants thereof. For polypeptides, this encompasses sequences that are identical to the Gene 216 polypeptide, as well as function-conservative and non-conservative variants thereof. Included are naturally-occurring mutations of Gene 216 causative of respiratory diseases or obesity, such as but not limited to mutations which cause altered protein levels or stability (e.g., decreased levels, increased levels, expression in an inappropriate tissue type, increased stability, and decreased stability).

As used herein, the “reference sequence” for Gene 216 is BAC1098L22 (SEQ ID NO:5). The BAC1098L22 sequence is also the source of the disclosed Gene 216 genomic sequence (SEQ ID NO:6). “Variant” sequences refer to nucleotide sequences (and the encoded amino acid sequences) that differ from the reference sequence at one or more positions. Non-limiting examples of variant sequences include the disclosed Gene 216 single nucleotide polymorphisms (SNPs), alternate splice variants, and the amino acid sequences encoded by these variants.

“Sequence-conservative” variants are those in which a change of one or more nucleotides in a given codon position results in no alteration in the amino acid encoded at that position (i.e., silent mutations). “Function-conservative” variants are those in which a change in one or more nucleotides in a given codon position results in a polypeptide sequence in which a given amino acid residue in the polypeptide has been replaced by a conservative amino acid substitution as described in detail herein. “Function-conservative” variants also include analogs of a given polypeptide and any polypeptides that have the ability to elicit antibodies specific to a designated polypeptide. “Non-conservative” variants are those in which a change in one or more nucleotides in a given codon position results in a polypeptide sequence in which a given amino acid residue in a polypeptide has been replaced by a non-conservative amino acid substitution as described hereinbelow. “Non-conservative” variants also include polypeptides comprising non-conservative amino acid substitutions.

As used herein, the term “ortholog” denotes a gene or polypeptide obtained from one species that has homology to an analogous gene or polypeptide from a different species. The term “paralog” denotes a gene or polypeptide obtained from a given species that has homology to a distinct gene or polypeptide from that same species. For example, the disclosed mouse and human Gene 216 sequences are orthologs, whereas human Gene 216 and human ADAM 19 are paralogs.

“Nucleic acid or “polynucleotide” as used herein refers to purine- and pyrimidine-containing polymers of any length, either polyribonucleotides or polydeoxyribonucleotide or mixed polyribo-polydeoxyribonucleotides. This includes single-and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases.

As used herein, “isolated” nucleic acids are nucleic acids separated away from other components (e.g., DNA, RNA, and protein) with which they are associated (e.g., as obtained from cells, chemical synthesis systems, or phage or nucleic acid libraries). Isolated nucleic acids are at least 60% free, preferably 75% free, and most preferably 90% free from other associated components. In accordance with the present invention, isolated nucleic acids can be obtained by methods described herein, or other established methods, including isolation from natural sources (e.g., cells, tissues, or organs), chemical synthesis, recombinant methods, combinations of recombinant and chemical methods, and library screening methods.

Nucleic acids referred to herein as “recombinant” are nucleic acids which have been produced by recombinant DNA methodology, including those nucleic acids that are generated by procedures which rely upon a method of artificial replication, such as the polymerase chain reaction (PCR) and/or cloning into a vector using restriction enzymes. Portions of recombinant nucleic acids which code for polypeptides can be identified and isolated by, for example, the method of M. Jasin et al., U.S. Pat. No. 4,952,501.

A “coding sequence” or a “protein-coding sequence” is a polynucleotide sequence capable of being transcribed into mRNA and/or capable of being translated into a polypeptide or peptide. The boundaries of the coding sequence are typically determined by a translation start codon at the 5′-terminus and a translation stop codon at the 3′-terminus.

A “complement” of a nucleic acid sequence as used herein refers to the “antisense” sequence that participates in Watson-Crick base-pairing with the original sequence.

A “probe” or “primer” refers to a nucleic acid or oligonucleotide that forms a hybrid structure with a sequence in a target region due to complementarily of the probe or primer sequence to at least one portion of the target region sequence.

Nucleic acids are “hybridizable” to each other when at least one strand of the nucleic acid can anneal to another nucleic acid strand under defined stringency conditions. Hybridization requires that the two nucleic acids contain substantially complementary sequences; depending on the stringency of hybridization, however, mismatches may be tolerated. The appropriate stringency for hybridizing nucleic acids depends on the length of the nucleic acids and the degree of complementarily, and can be determined in accordance with the methods described herein.

As used herein, “portion” and “fragment” are synonymous. A “portion” as used with regard to a nucleic acid or polynucleotide, refers to fragments of that nucleic acid or polynucleotide. The fragments can range in size from 8 nucleotides to all but one nucleotide of the entire Gene 216 sequence. Preferably, The fragments are at least 8 to 10 nucleotides in length; more preferably at least 12 nucleotides in length; still more preferably at least 15 to 20 nucleotides in length; yet more preferably at least 25 nucleotides in length; and most preferably at least 35 to 55 nucleotides in length.

“cDNA” refers to complementary or copy DNA produced from an RNA template by the action of RNA-dependent DNA polymerase (reverse transcriptase). Thus, a “cDNA clone” means a duplex DNA sequence complementary to an RNA molecule of interest, included in a cloning vector or PCR amplified. This term includes genes from which the intervening sequences have been removed.

“Cloning” refers to the use of recombination techniques to insert a particular gene or other DNA sequence into a vector molecule. In order to successfully clone a desired gene, it is necessary to use methods for generating DNA fragments, for joining the fragments to vector molecules, for introducing the composite DNA molecule into a host cell in which it can replicate, and for selecting the clone having the target gene from amongst the recipient host cells.

“cDNA library” refers to a collection of recombinant DNA molecules containing cDNA inserts that together comprise essentially all of the expressed genes of an organism. A cDNA library can be prepared by methods known to one skilled in the art (see, e.g., Cowell and Austin, 1997, “cDNA Library Protocols,” Methods in Molecular Biology). Generally, RNA is first isolated from the cells of the desired organism, and the RNA is used to prepare cDNA molecules.

“Cloning vector” refers to a plasmid or phage DNA or other DNA that is able to replicate in a host cell. The cloning vector is typically characterized by one or more endonuclease recognition sites at which such DNA sequences may be cut in a determinable fashion without loss of an essential biological function of the DNA, which may contain a marker suitable for use in the identification of cells containing the vector.

“Regulatory sequence” refers to a nucleic acid sequence that controls or regulates expression of structural genes when operably linked to those genes. These include, for example, the lac systems, the trp system, major operator and promoter regions of the phage lambda, the control region of fd coat protein and other sequences known to control the expression of genes in prokaryotic or eukaryotic cells. Regulatory sequences will vary depending on whether the vector is designed to express the operably linked gene in a prokaryotic or eukaryotic host, and may contain transcriptional elements such as enhancer elements, termination sequences, tissue-specificity elements and/or translational initiation and termination sites.

“Expression vector” refers to a vehicle or plasmid that is capable of expressing a gene that has been cloned into it, after transformation or integration in a host cell. The cloned gene is usually placed under the control of (i.e., operably linked to) a regulatory sequence.

“Operably linked” means that the promoter controls the initiation of expression of the gene. A promoter is operably linked to a sequence of proximal DNA if upon introduction into a host cell the promoter determines the transcription of the proximal DNA sequence(s) into one or more species of RNA. A promoter is operably linked to a DNA sequence if the promoter is capable of initiating transcription of that DNA sequence.

“Host” includes prokaryotes and eukaryotes. The term includes an organism or cell that is the recipient of an expression vector (e.g., autonomously replicating or integrating vector).

“Amplification” of nucleic acids refers to methods such as polymerase chain reaction (PCR), ligation amplification (or ligase chain reaction, LCR) and amplification methods based on the use of Q-beta replicase. These methods are well known in the art and described, for example, in U.S. Pat. Nos. 4,683,195 and 4,683,202. Reagents and hardware for conducting PCR are commercially available. Primers useful for amplifying sequences from the disorder region are preferably complementary to, and preferably hybridize specifically to, sequences in the 20p13-p12 region or in regions that flank a target region therein. Gene 216 generated by amplification may be sequenced directly. Alternatively, the amplified sequence(s) may be cloned prior to sequence analysis.

“Gene” refers to a DNA sequence that encodes through its template or messenger RNA a sequence of amino acids characteristic of a specific peptide, polypeptide, or protein. The term “gene” as used herein with reference to genomic DNA includes intervening, non-coding regions, as well as regulatory regions, and can include 5′ and 3′ ends.

A gene sequence is “wild-type” if such sequence is usually found in individuals unaffected by the disease or condition of interest. However, environmental factors and other genes can also play an important role in the ultimate determination of the disease. In the context of complex diseases involving multiple genes (“oligogenic disease”), the “wild type”, or normal sequence can also be associated with a measurable risk or susceptibility, receiving its reference status based on its frequency in the general population. As used herein, “wild-type Gene 216” refers to the reference sequence, BAC1098L22 (SEQ ID NO:5). The wild-type Gene 216 sequence was used to identify the variants (single nucleotide polymorphisms) described in detail herein.

A gene sequence is a “mutant” sequence if it differs from the wild-type sequence. For example, a Gene 216 nucleic acid containing a single nucleotide polymorphism is a mutant sequence. In some cases, the individual carrying such gene has increased susceptibility toward the disease or condition of interest. In other cases, the “mutant” sequence might also refer to a sequence that decreases the susceptibilty toward a disease or condition of interest, and thus acting in a protective manner. Also a gene is a “mutant” gene if too much (“overexpressed”) or too little (“underexpressed”) of such gene is expressed in the tissues in which such gene is normally expressed, thereby causing the disease or condition of interest.

A nucleic acid or fragment thereof is “substantially homologous” to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases.

Alternatively, substantial homology exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90% (M. Kanehisa, 1984, Nucl. Acids Res. 11:203-213). The length of homology comparison, as described, may be over longer stretches, and in certain embodiments will often be over a stretch of at least 14 nucleotides, usually at least 20 nucleotides, more usually at least 24 nucleotides, typically at least 28 nucleotides, more typically at least 32 nucleotides, and preferably at least 36 or more nucleotides.

As used herein, the terms “protein” and “polypeptide” are synonymous. “Peptides” are defined as fragments or portions of polypeptides, preferably fragments or portions having at least one functional activity (e.g., proteolysis, adhesion, fusion, antigenic, or intracellular activity) as the complete polypeptide sequence.

“Isolated” polypeptides or peptides are those that are separated from other components (e.g., DNA, RNA, and other polypeptides or peptides) with which they are associated (e.g., as obtained from cells, translation systems, or chemical synthesis systems). In a preferred embodiment, isolated polypeptides or peptides are at least 10% pure; more preferably, 80 or 90% pure. Isolated polypeptides and peptides include those obtained by methods described herein, or other established methods, including isolation from natural sources (e.g., cells, tissues, or organs), chemical synthesis, recombinant methods, or combinations of recombinant and chemical methods. Proteins or polypeptides referred to herein as “recombinant” are proteins or polypeptides produced by the expression of recombinant nucleic acids.

A “portion” as used herein with regard to a protein or polypeptide, refers to fragments of that protein or polypeptide. The fragments can range in size from 5 amino acid residues to all but one residue of the entire protein sequence. Thus, a portion or fragment can be at least 5, 5-50, 50-100, 100-200, 200-400, 400-800, or more consecutive amino acid residues of a Gene 216 protein or polypeptide, for example, SEQ ID NO:4 or SEQ ID NO:363.

An “immunogenic component”, is a moiety that is capable of eliciting a humoral and/or cellular immune response in a host animal.

An “antigenic component” is a moiety that binds to its specific antibody with sufficiently high affinity to form a detectable antigen-antibody complex.

A “sample” as used herein refers to a biological sample, such as, for example, tissue or fluid isolated from an individual (including, without limitation, plasma, serum, cerebrospinal fluid, lymph, tears, saliva, milk, pus, and tissue exudates and secretions) or from in vitro cell culture constituents, as well as samples obtained from, for example, a laboratory procedure.

“Antibodies” refer to polyclonal and/or monoclonal antibodies and fragments thereof, and immunologic binding equivalents thereof, that can bind to asthma proteins and fragments thereof or to nucleic acid sequences from the 20p13-p12 region, particularly from the asthma locus or a portion thereof. The term antibody is used both to refer to a homogeneous molecular entity, or a mixture such as a serum product made up of a plurality of different molecular entities. Proteins may be prepared synthetically in a protein synthesizer and coupled to a carrier molecule and injected over several months into rabbits. Rabbit sera is tested for immunoreactivity to the protein or fragment. Monoclonal antibodies may be made by injecting mice with the proteins, or fragments thereof. Monoclonal antibodies will be screened by ELISA and tested for specific immunoreactivity with protein or fragments thereof. (Harlow et al., 1988, Antibodies: A Laboratory Manual, Cold Spring Harbor Laboratory, Cold Spring Harbor, N.Y.). These antibodies will be useful in assays as well as therapeutics.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (A. M. Lesk (ed), 1988, Computational Molecular Biology, Oxford University Press, NY; D. W. Smith (ed), 1993, Biocomputing. Informatics and Genome Projects, Academic Press, NY; A. M. Griffin and H. G. Griffin, H. G (eds), 1994, Computer Analysis of Sequence Data, Part I, Humana Press, NJ; G. von Heinje, 1987, Sequence Analysis in Molecular Biology, Academic Press; and M. Gribskov and J. Devereux (eds), 1991, Sequence Analysis Primer, M Stockton Press, NY; H. Carillo and D. Lipman, 1988, SIAM J. Applied Math., 48:1073.

Technical and scientific terms used herein have the meanings commonly understood by one of ordinary skill in the art to which the present invention pertains, unless otherwise defined. Reference is made herein to various methodologies known to those of skill in the art. Publications and other materials setting forth such known methodologies to which reference is made are incorporated herein by reference in their entireties as though set forth in full.

Standard reference works setting forth the general principles of recombinant DNA technology include J. Sambrook et al., 1989, Molecular Cloning: A Laboratory Manual, 2d Ed., Cold Spring Harbor Laboratory Press, Cold Spring Harbor, N.Y.; P. B. Kaufman et al., (eds), 1995, Handbook of Molecular and Cellular Methods in Biology and Medicine, CRC Press, Boca Raton; M. J. McPherson (ed), 1991, Directed Mutagenesis: A Practical Approach, IRL Press, Oxford; J. Jones, 1992, Amino Acid and Peptide Synthesis, Oxford Science Publications, Oxford; B. M. Austen and O. M. R. Westwood, 1991, Protein Targeting and Secretion, IRL Press, Oxford; D. N Glover (ed), 1985, DNA Cloning, Volumes I and II; M. J. Gait (ed), 1984, Oligonucleotide Synthesis; B. D. Hames and S. J. Higgins (eds), 1984, Nucleic Acid Hybridization; Wu and Grossman (eds), Methods in Enzymology (Academic Press, Inc.), Vol. 154 and Vol. 155; Quirke and Taylor (eds), 1991, PCR-A Practical Approach; Hames and Higgins (eds), 1984, Transcription and Translation; R. I. Freshney (ed), 1986, Animal Cell Culture; Immobilized Cells and Enzymes, 1986, IRL Press; Perbal, 1984, A Practical Guide to Molecular Cloning; J. H. Miller and M. P. Calos (eds), 1987, Gene Transfer Vectors for Mammalian Cells, Cold Spring Harbor Laboratory Press; M. J. Bishop (ed), 1998, Guide to Human Genome Computing, 2d Ed., Academic Press, San Diego, Calif.; L. F. Peruski and A. H. Peruski, 1997, The Internet and the New Biology: Tools for Genomic and Molecular Research, American Society for Microbiology, Washington, D.C.

Standard reference works setting forth the general principles of immunology include S. Sell, 1996, Immunology, Immunopathology & Immunity, 5th Ed., Appleton & Lange, Publ., Stamford, Conn.; D. Male et al., 1996, Advanced Immunology, 3d Ed., Times Mirror Int'l Publishers Ltd., Publ., London; D. P. Stites and A. I. Terr, 1991, Basic and Clinical Immunology, 7th Ed., Appleton & Lange, Publ., Norwalk, Conn.; and A. K. Abbas et al., 1991,

Cellular and Molecular Immunology, W. B. Saunders Co., Publ., Philadelphia, Pa. Any suitable materials and/or methods known to those of skill can be utilized in carrying out the present invention; however, preferred materials and/or methods are described. Materials, reagents, and the like to which reference is made in the following description and examples are generally obtainable from commercial sources, and specific vendors are cited herein.

Nucleic Acids

The present invention relates to isolated Gene 216 nucleic acids comprising genomic DNA within BAC RPCI_{—}1098L22 (e.g., SEQ ID NO:5), the corresponding cDNA sequences (e.g., SEQ ID NO:1 or SEQ ID NO:3), RNA, fragments of the genomic, cDNA, or RNA nucleic acids comprising 20, 40, 60, 100, 200, 500 or more contiguous nucleotides, and the complements thereof. Closely related variants are also included as part of this invention, as well as nucleic acids sharing at least 50, 60, 70, 80, or 90% identity with the nucleic acids described above, and nucleic acids which would be identical to a Gene 216 nucleic acids except for one or a few substitutions, deletions, or additions.

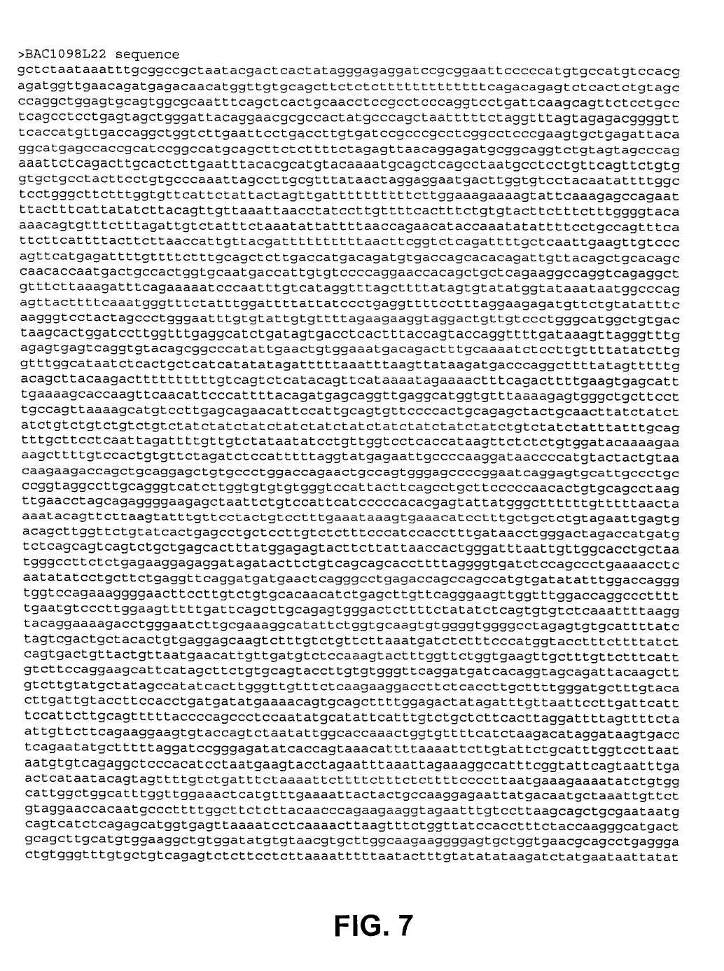

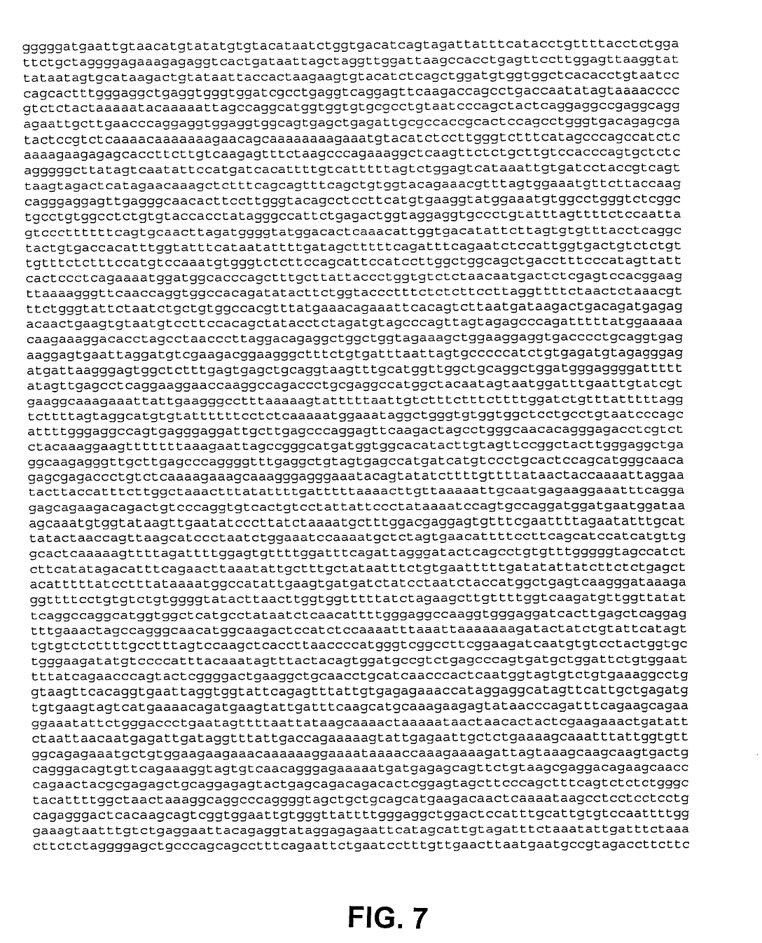

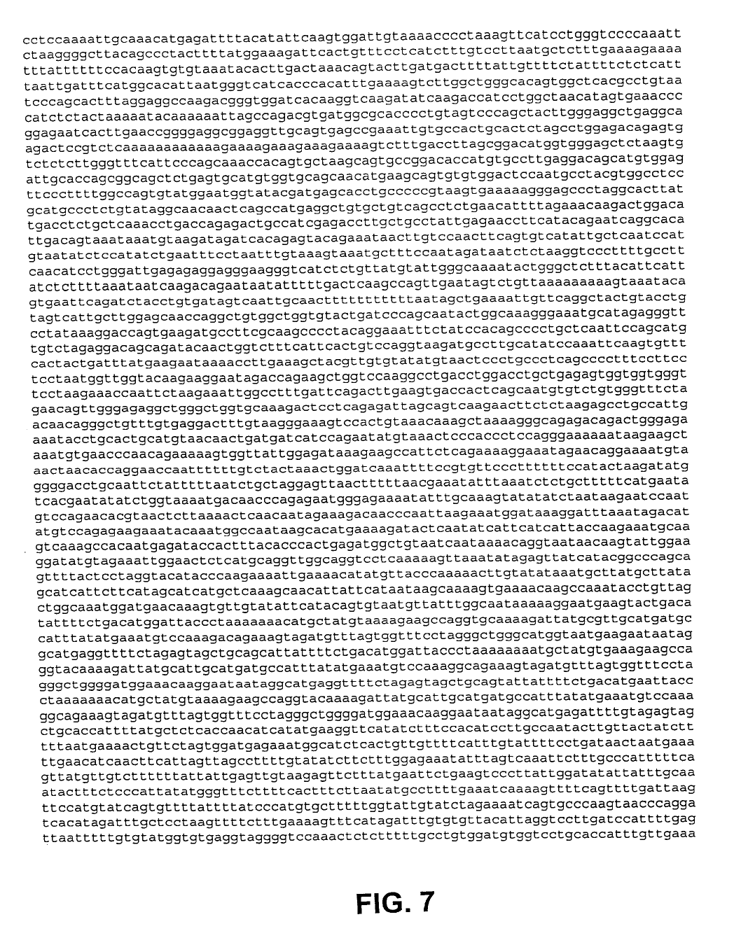

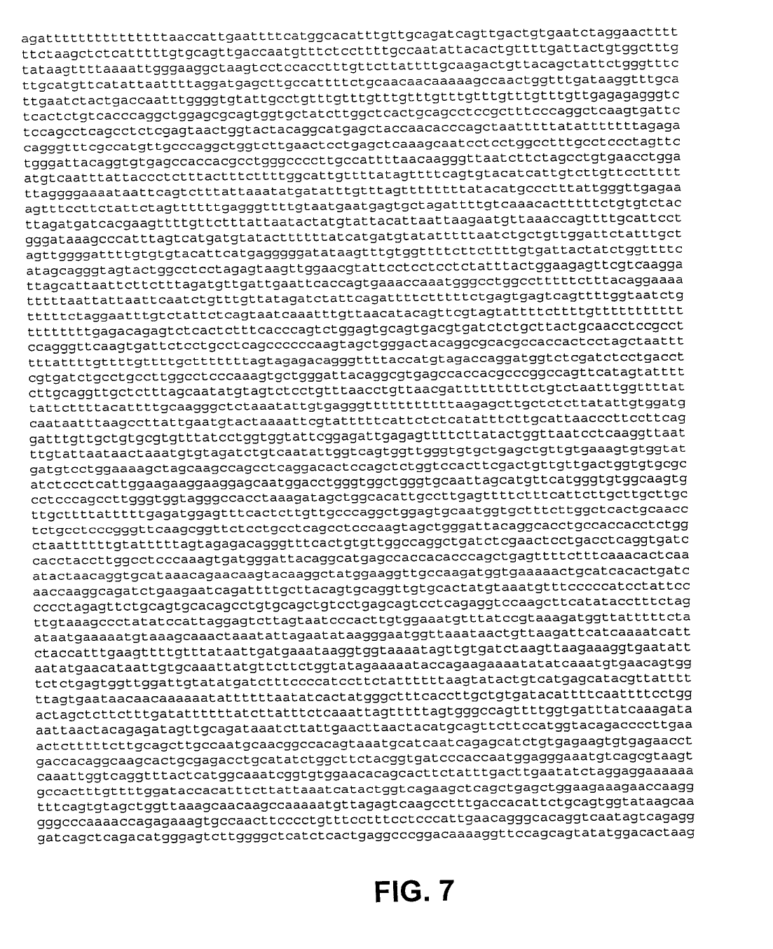

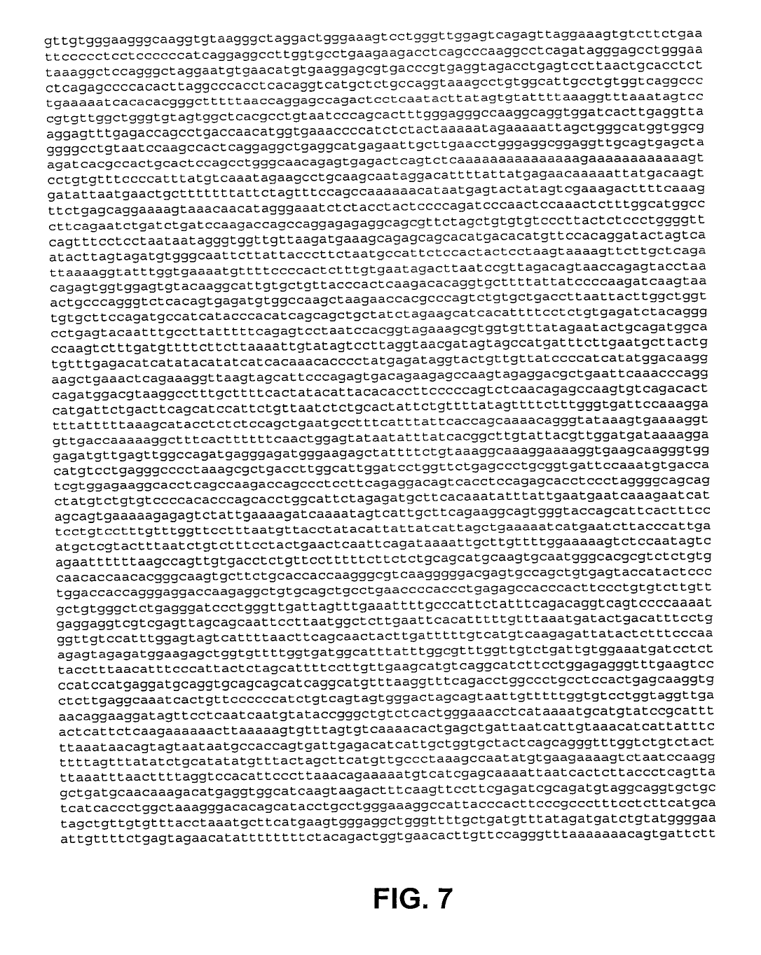

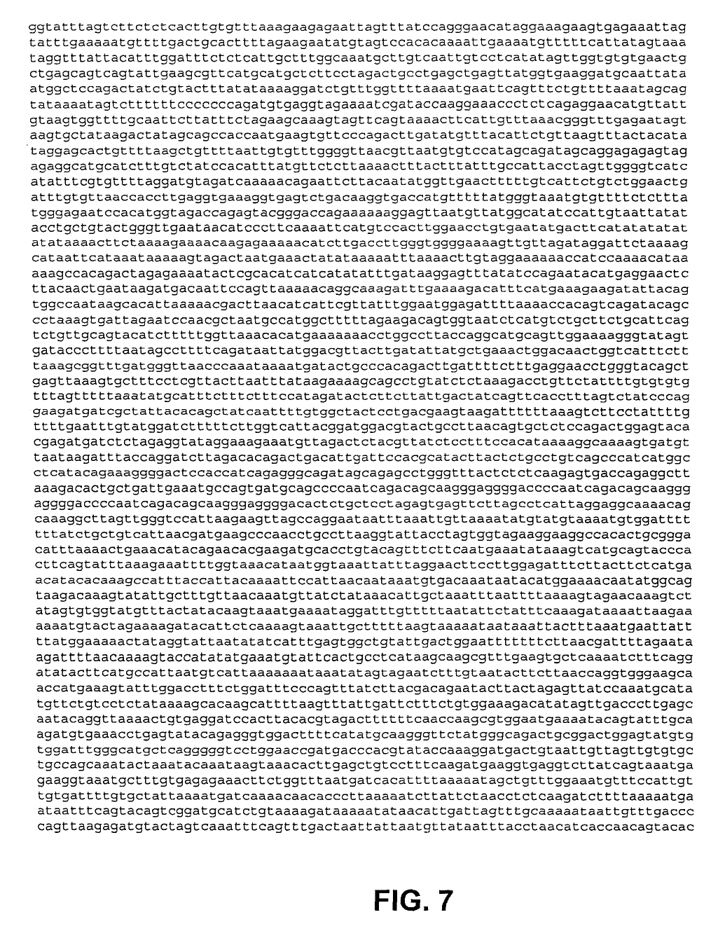

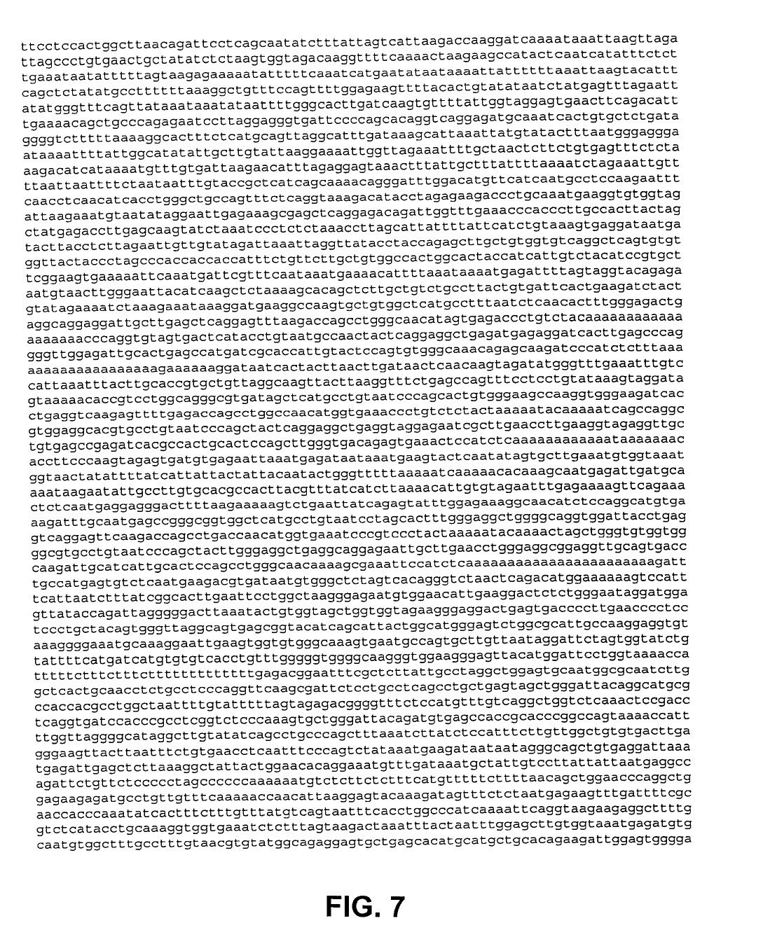

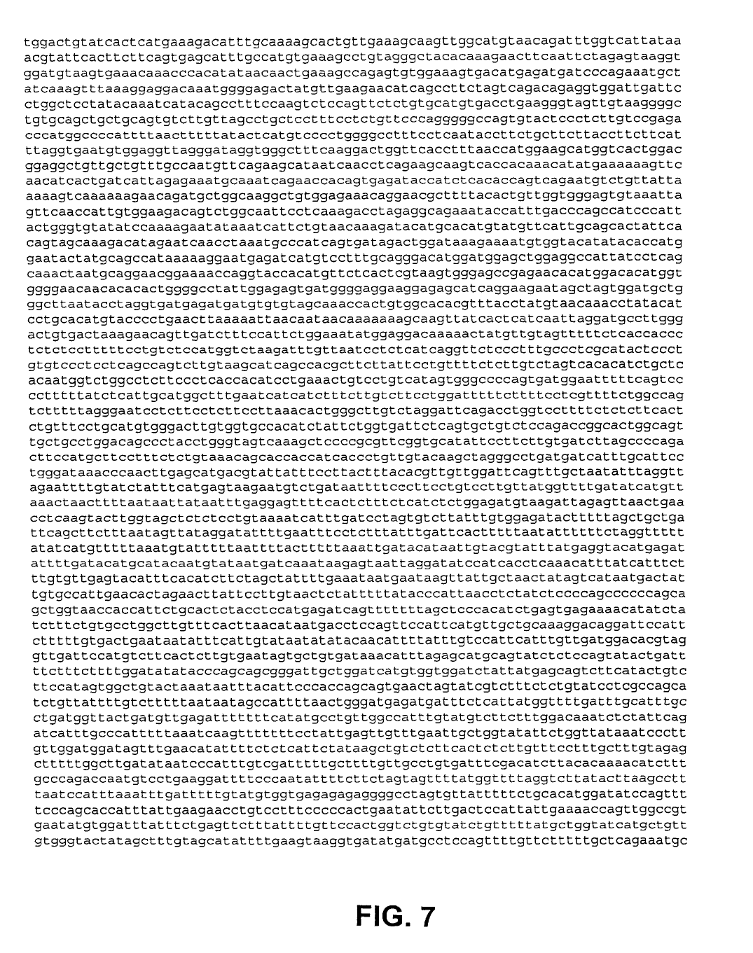

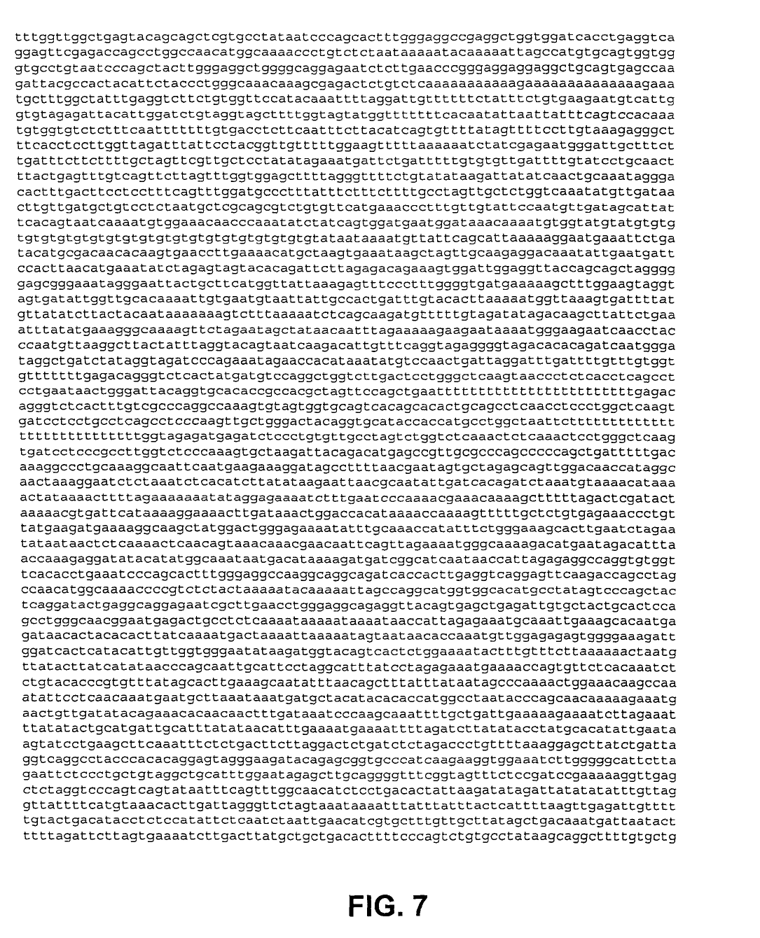

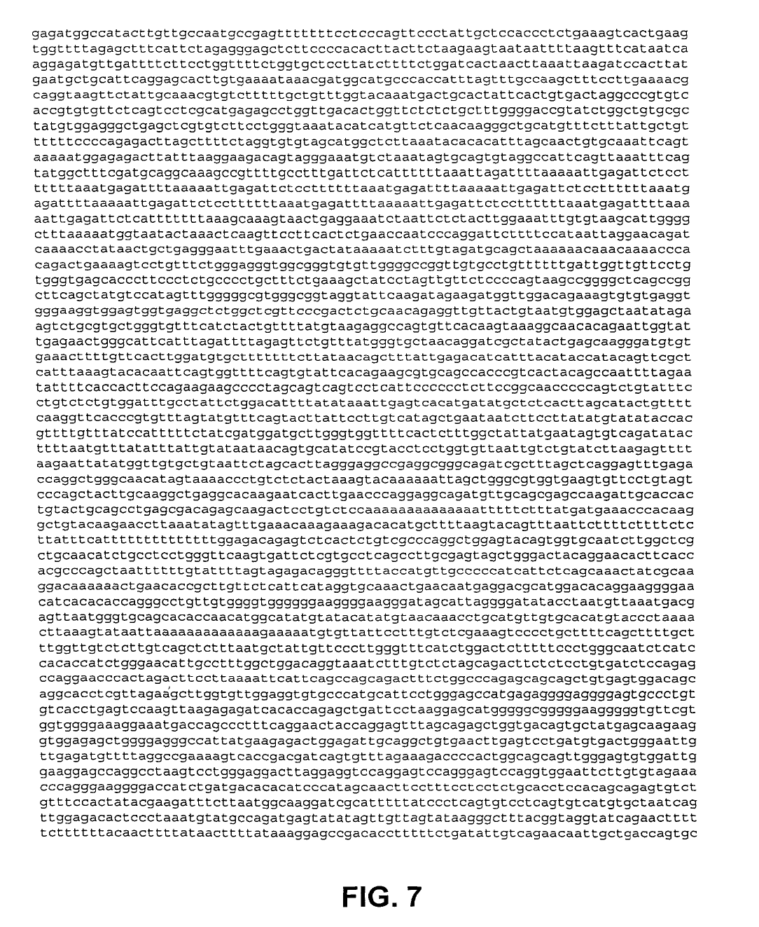

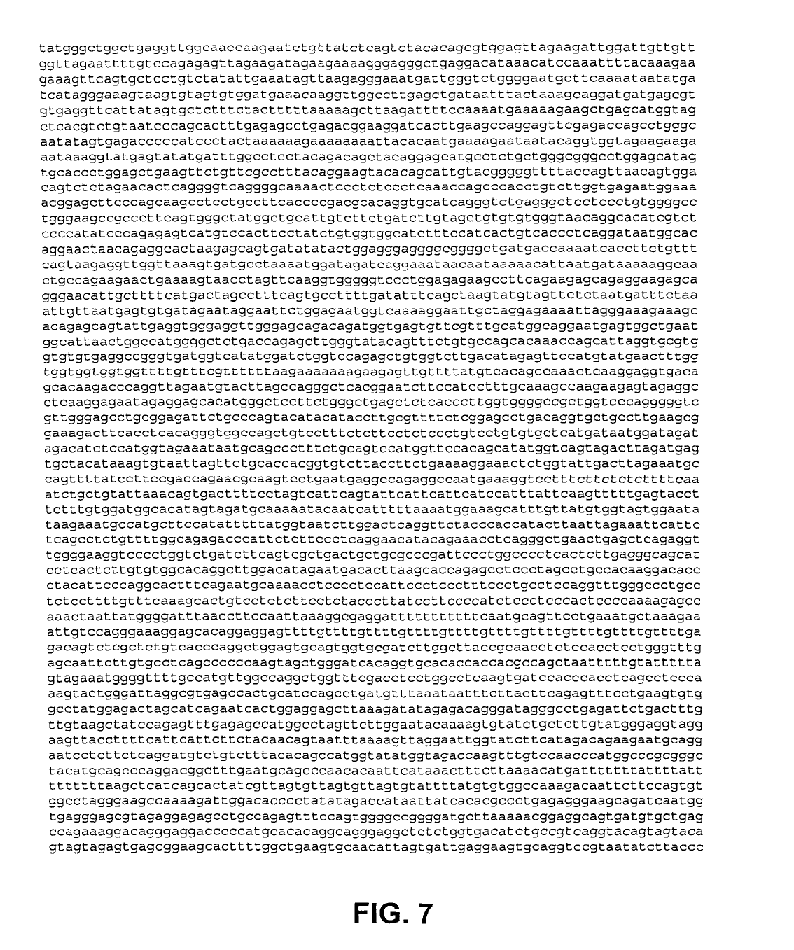

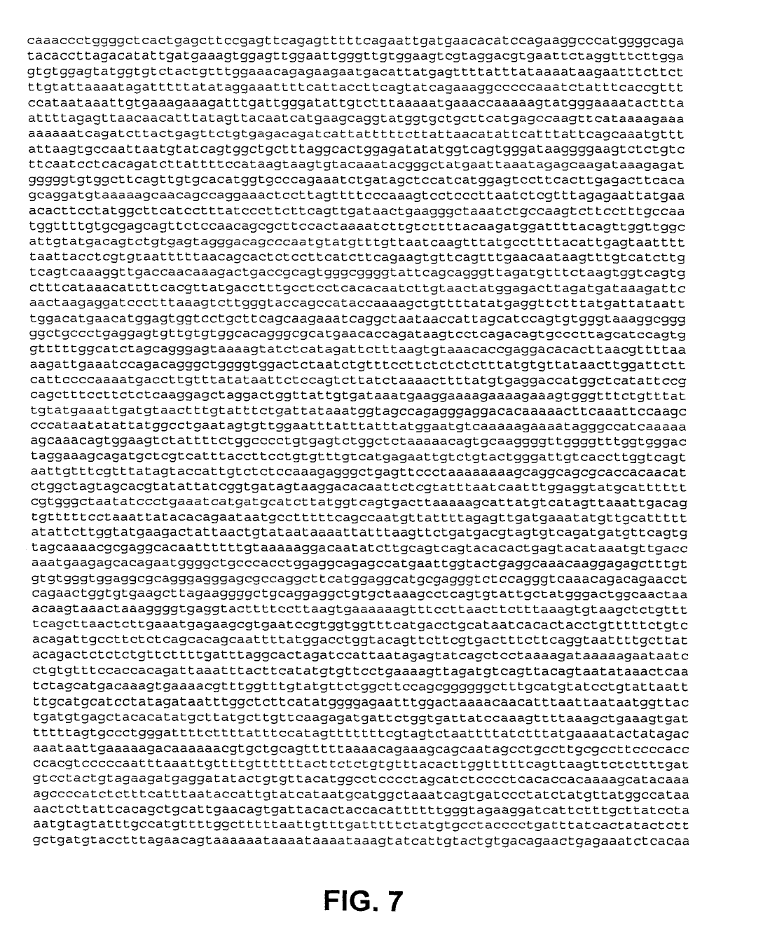

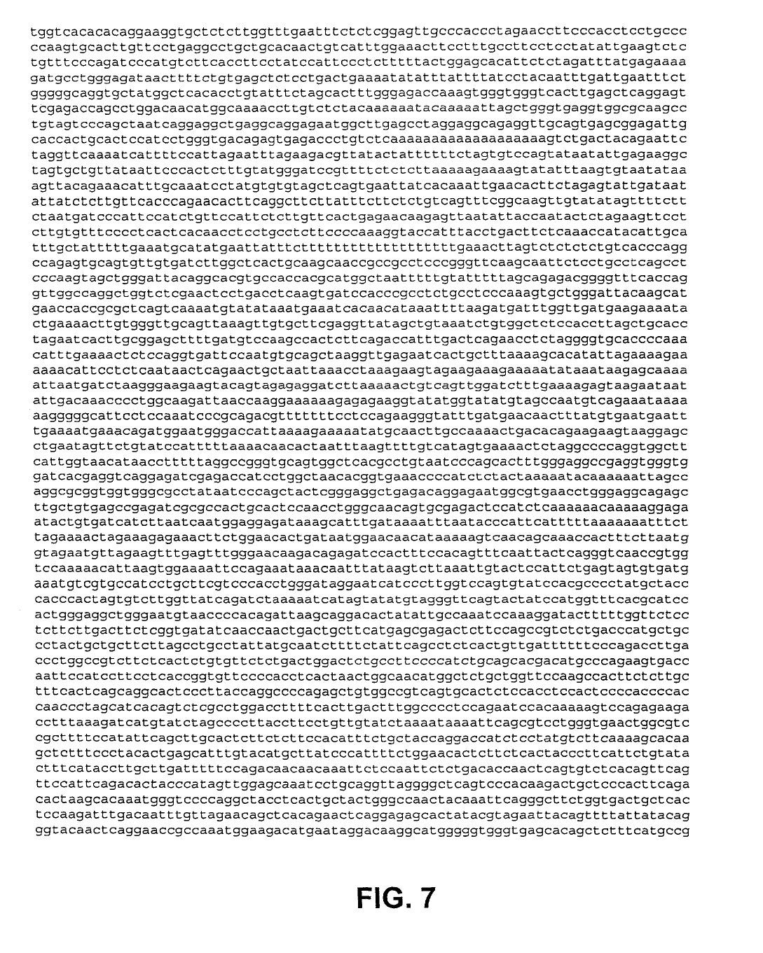

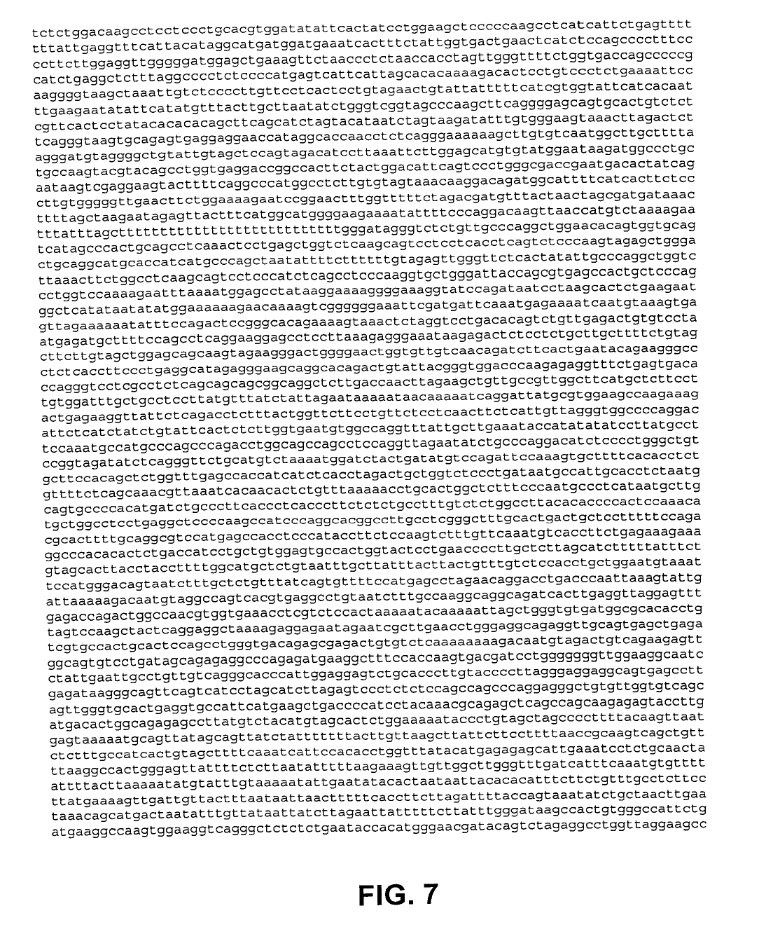

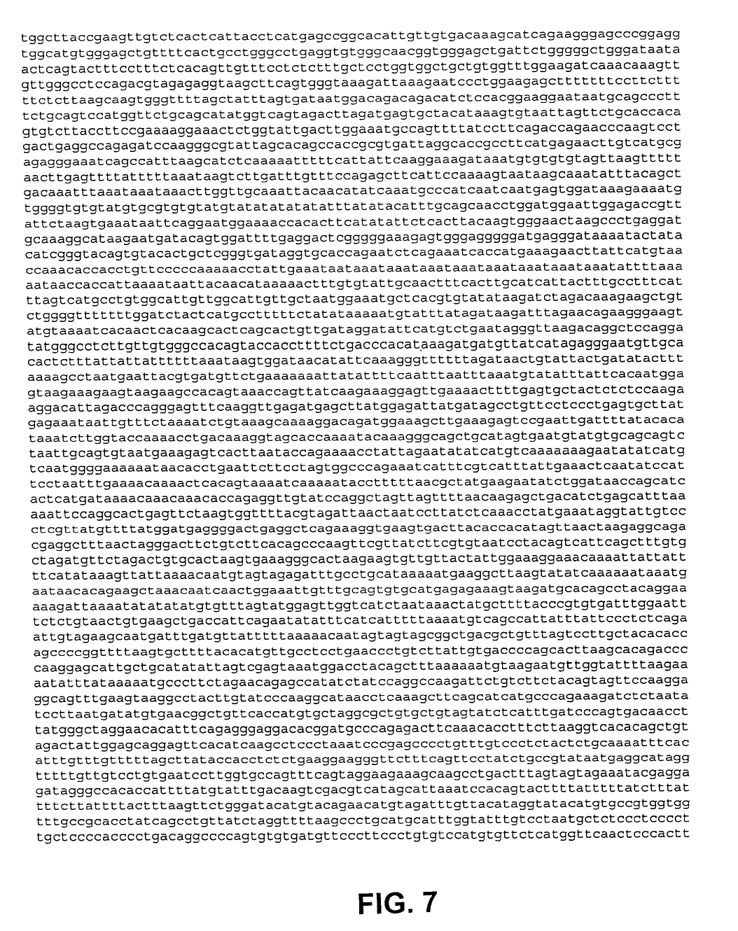

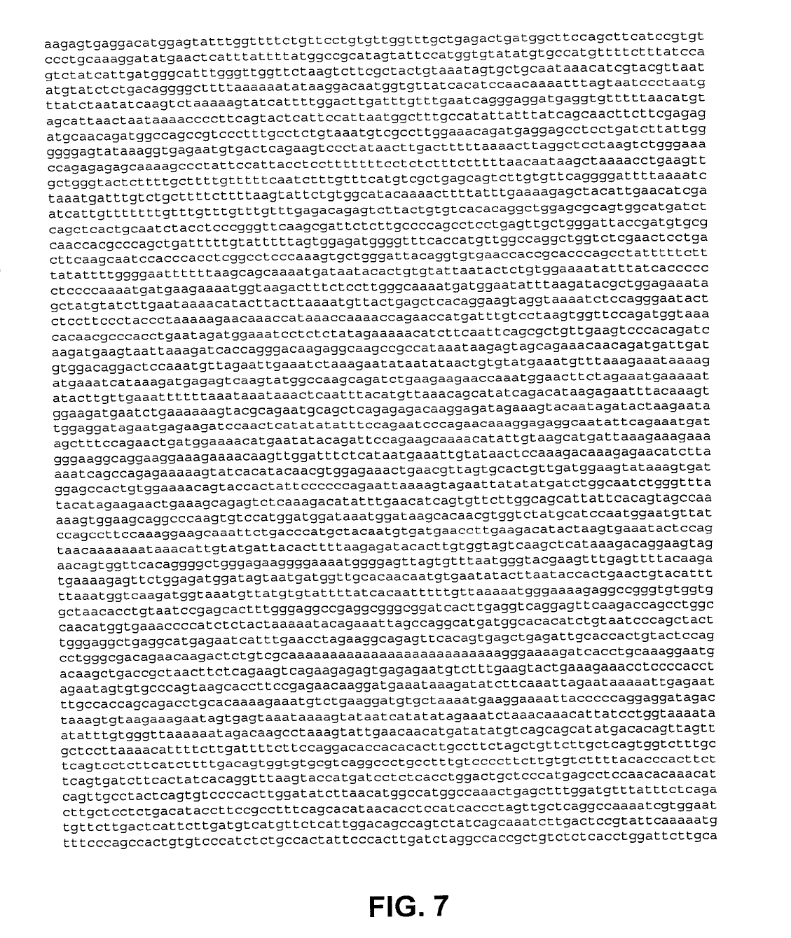

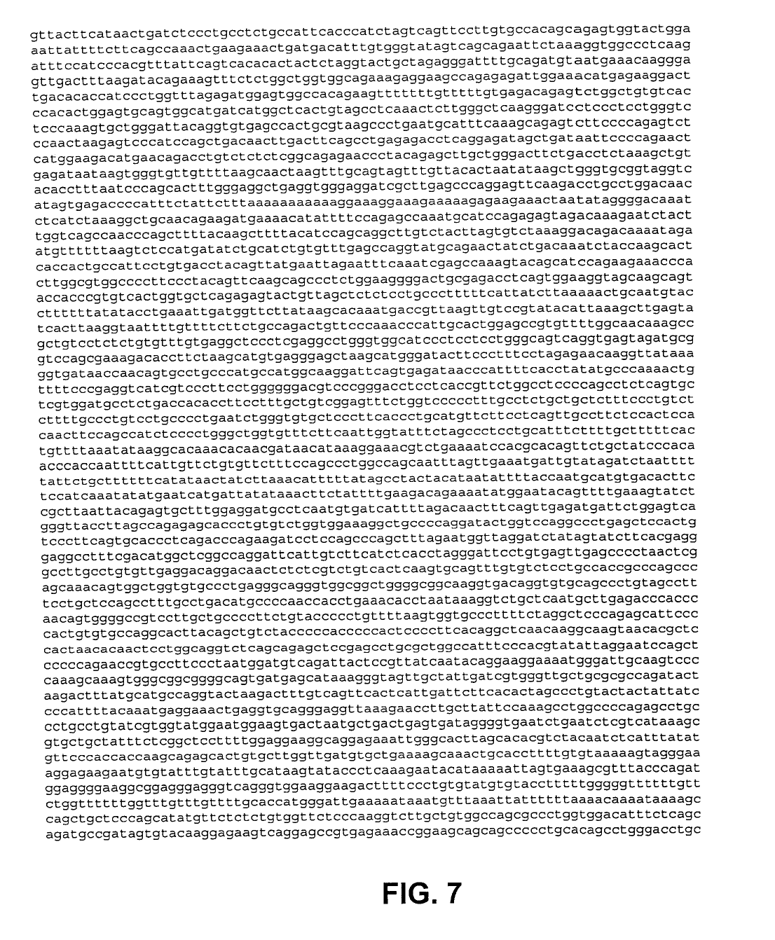

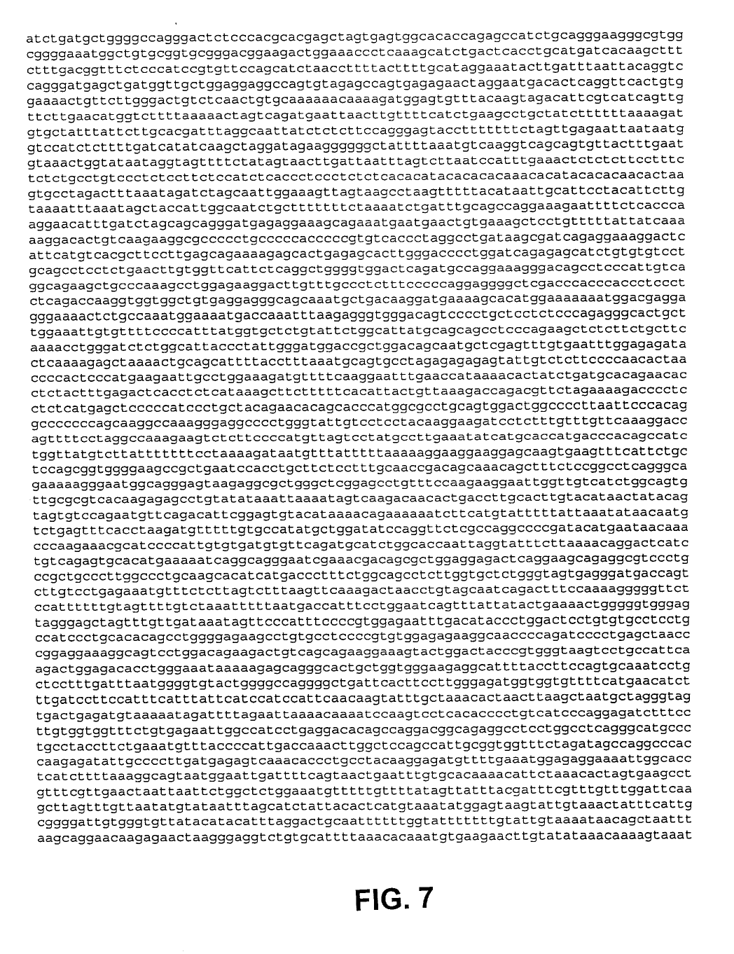

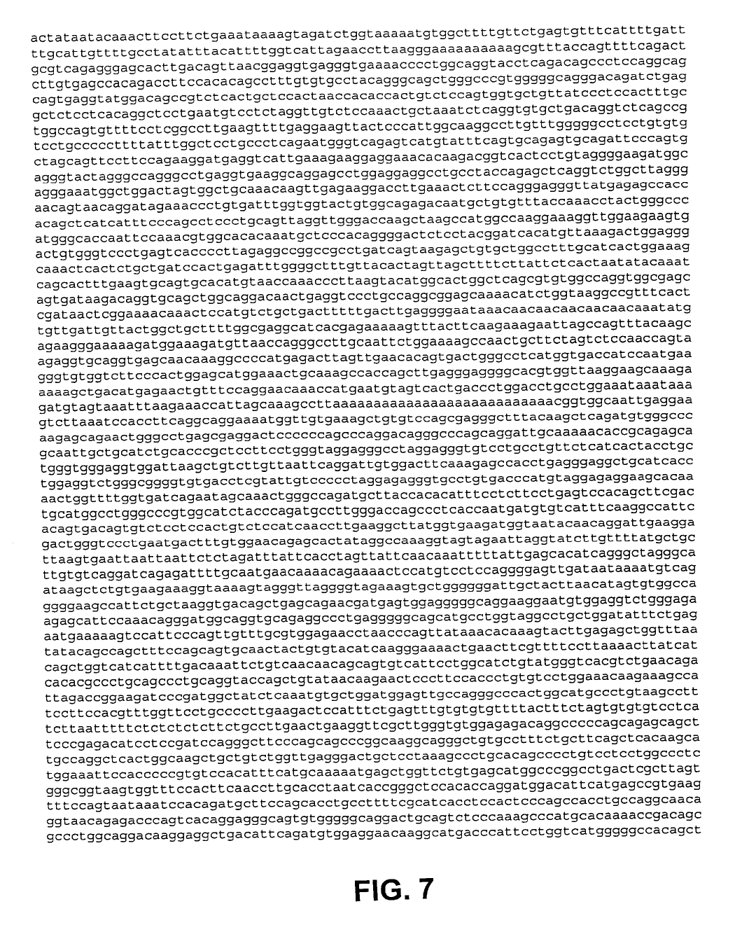

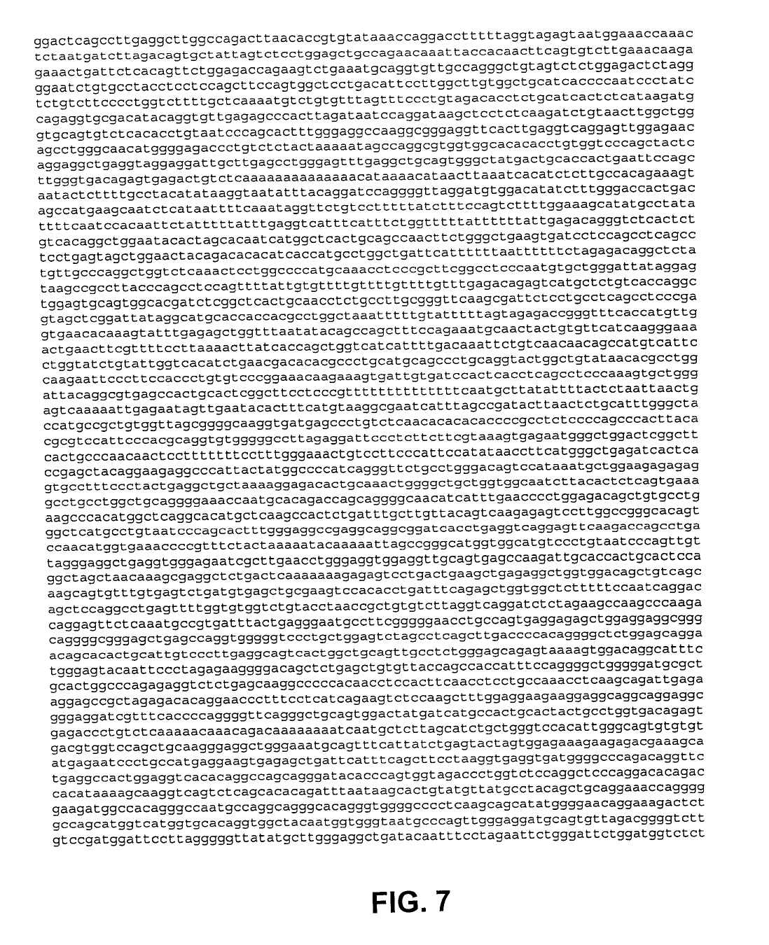

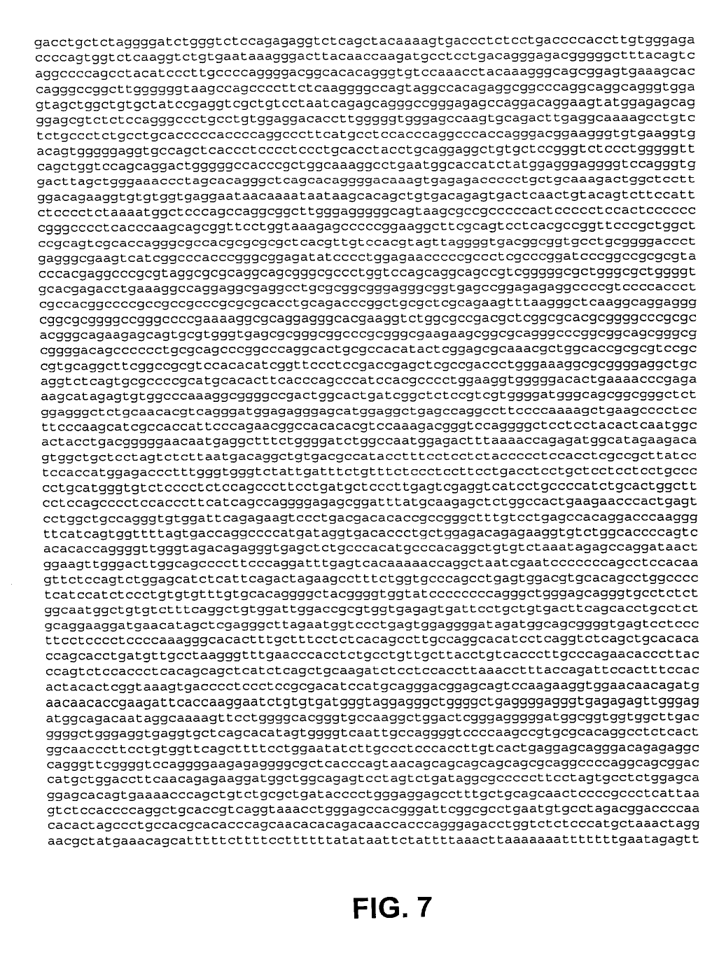

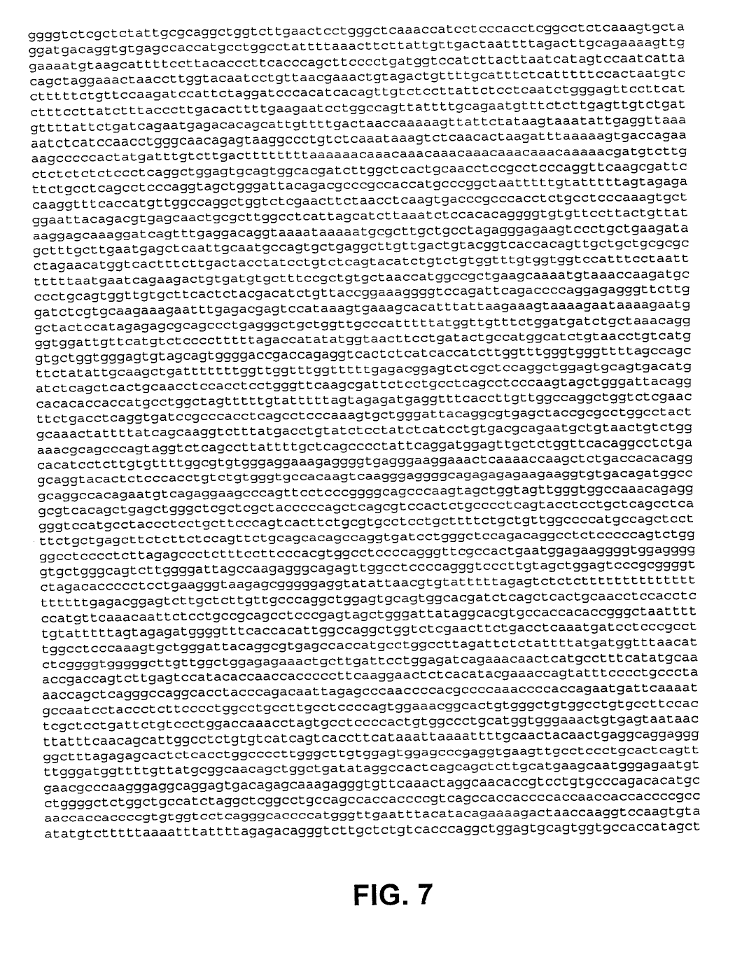

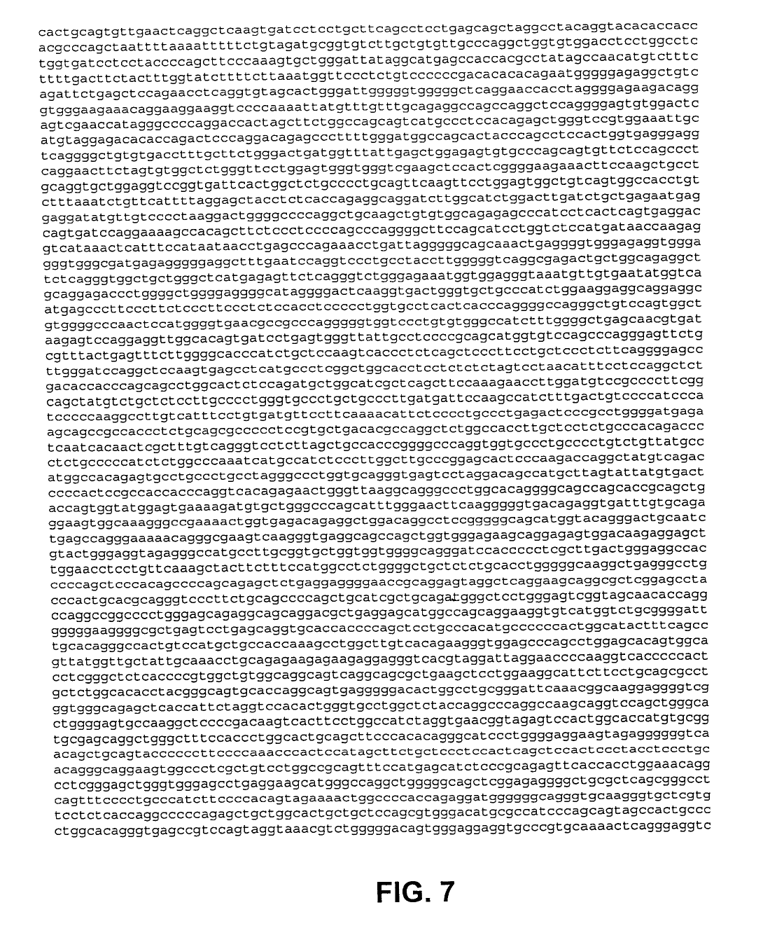

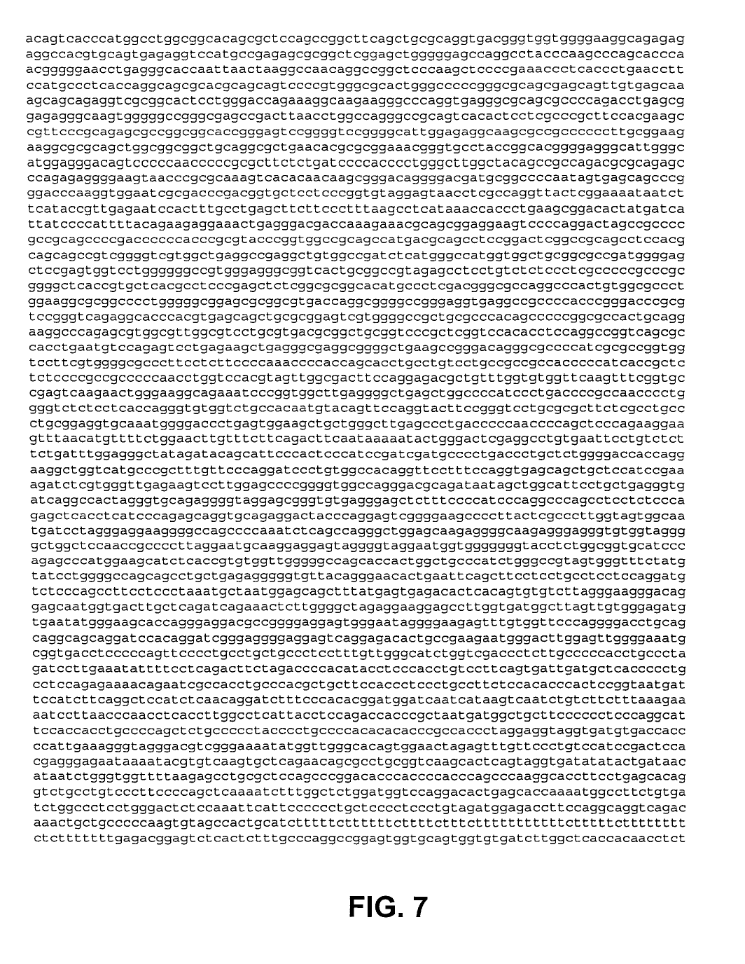

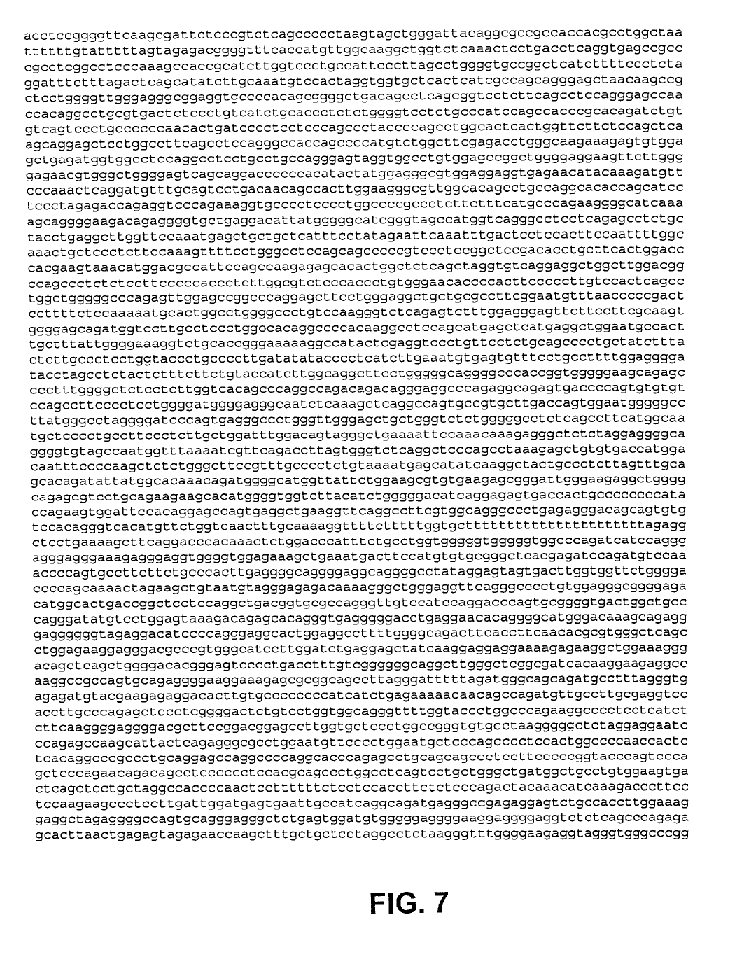

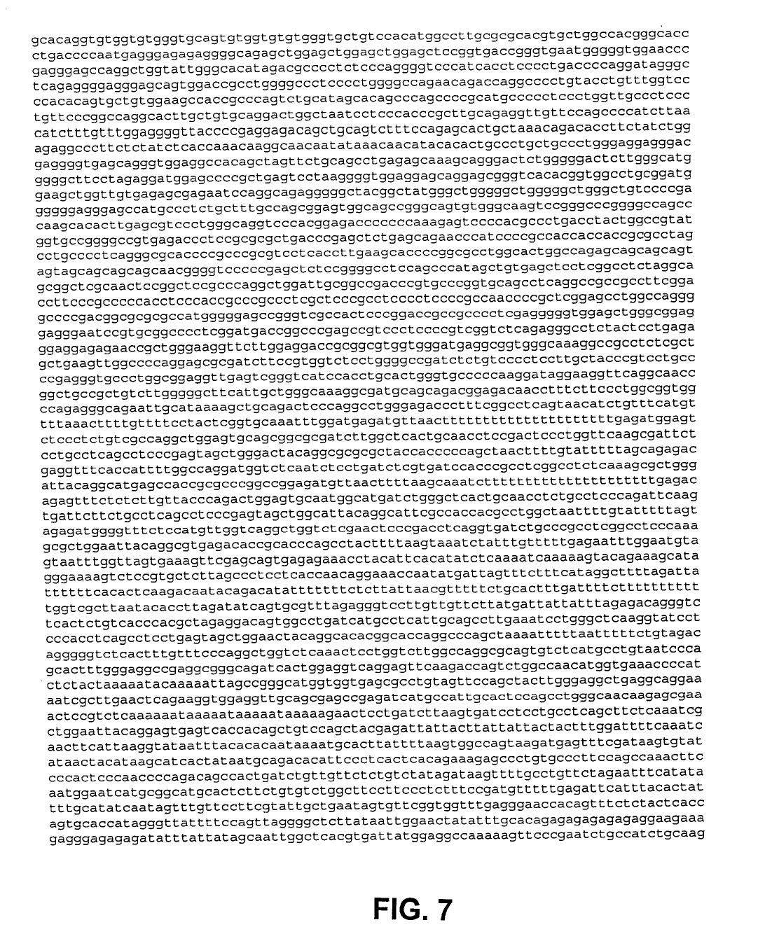

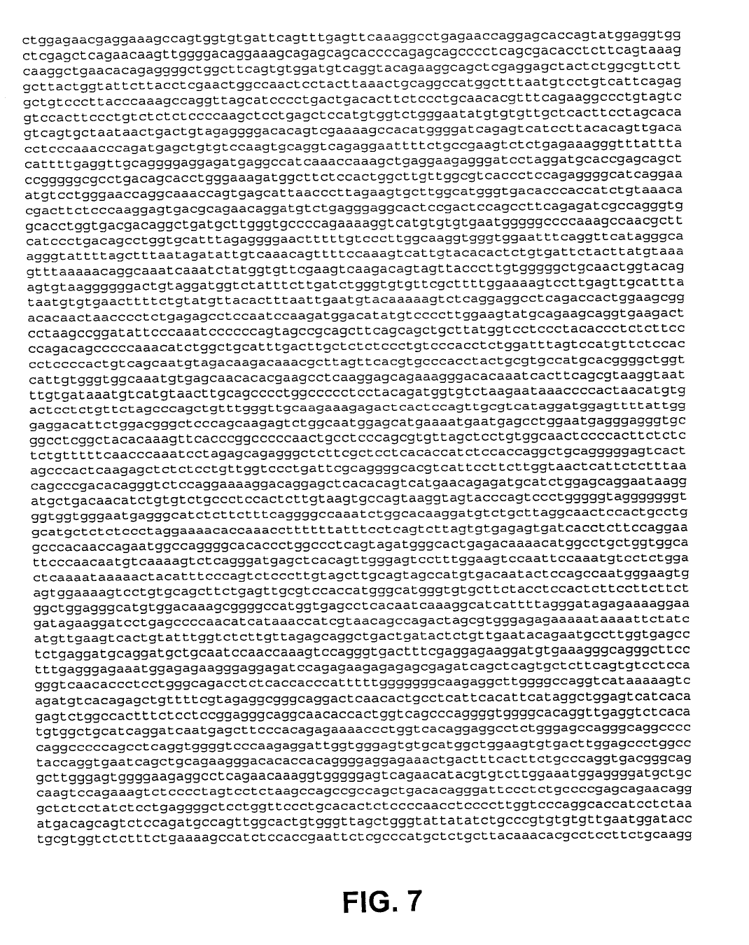

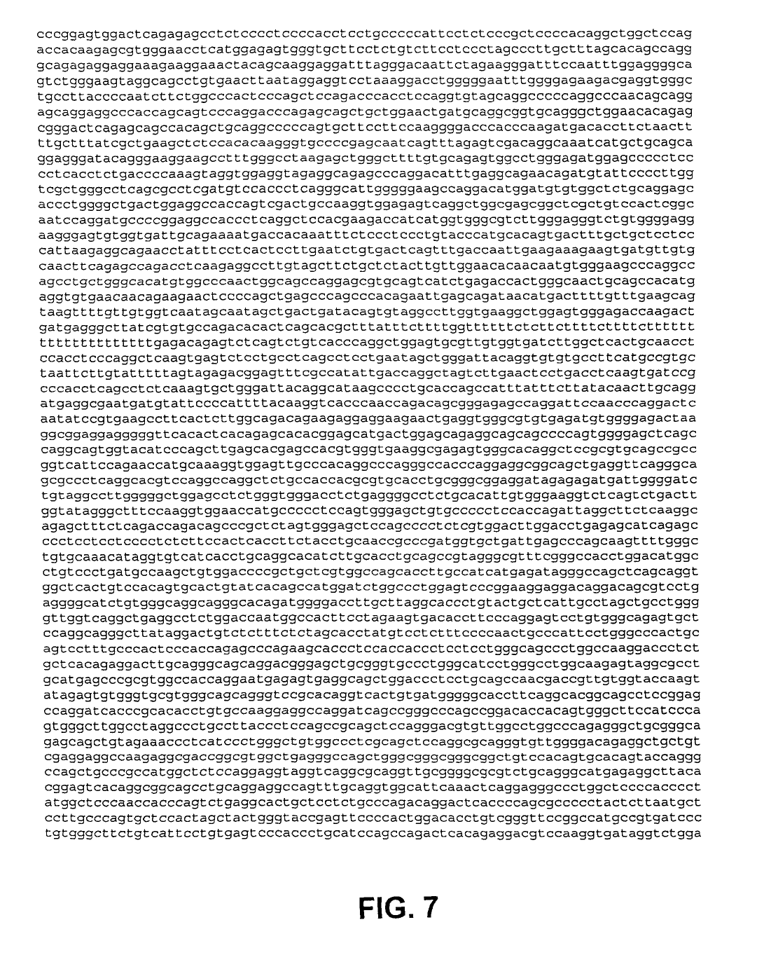

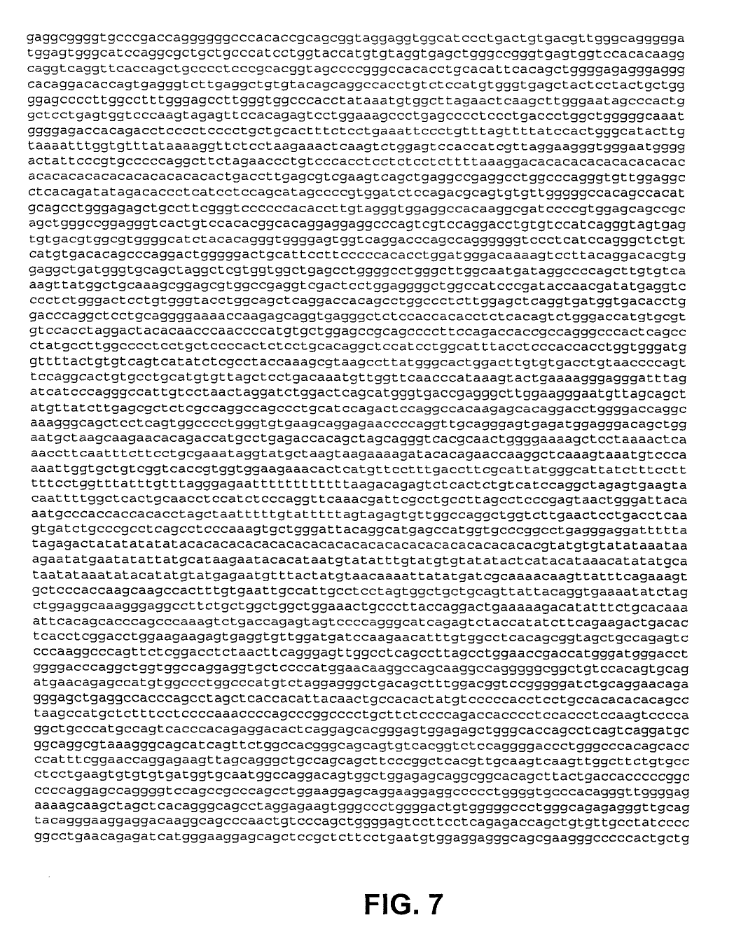

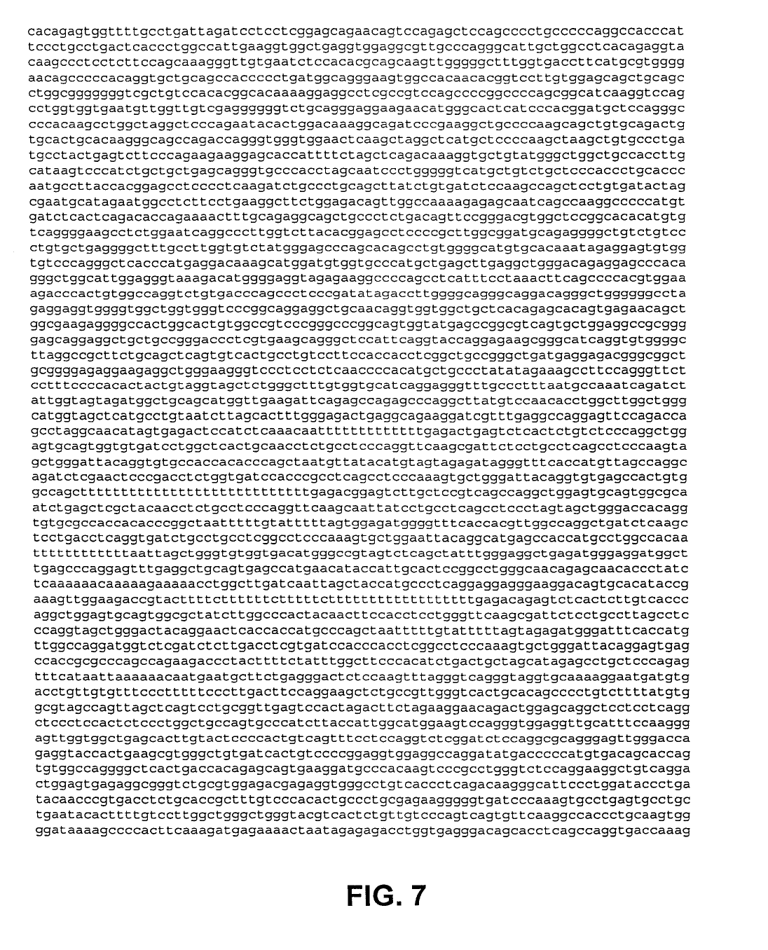

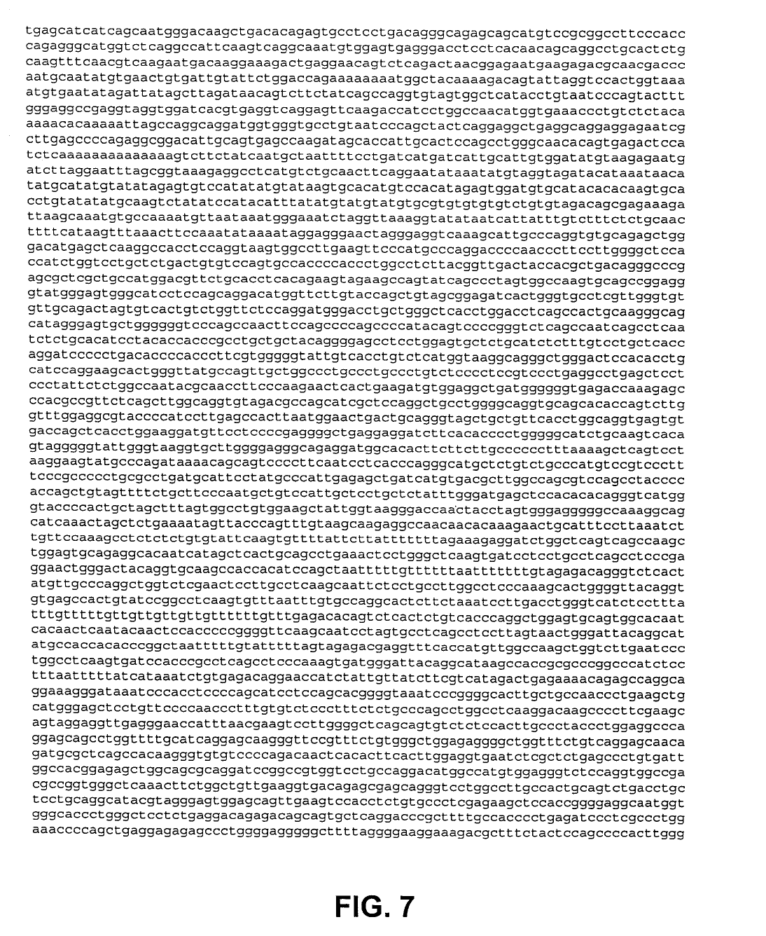

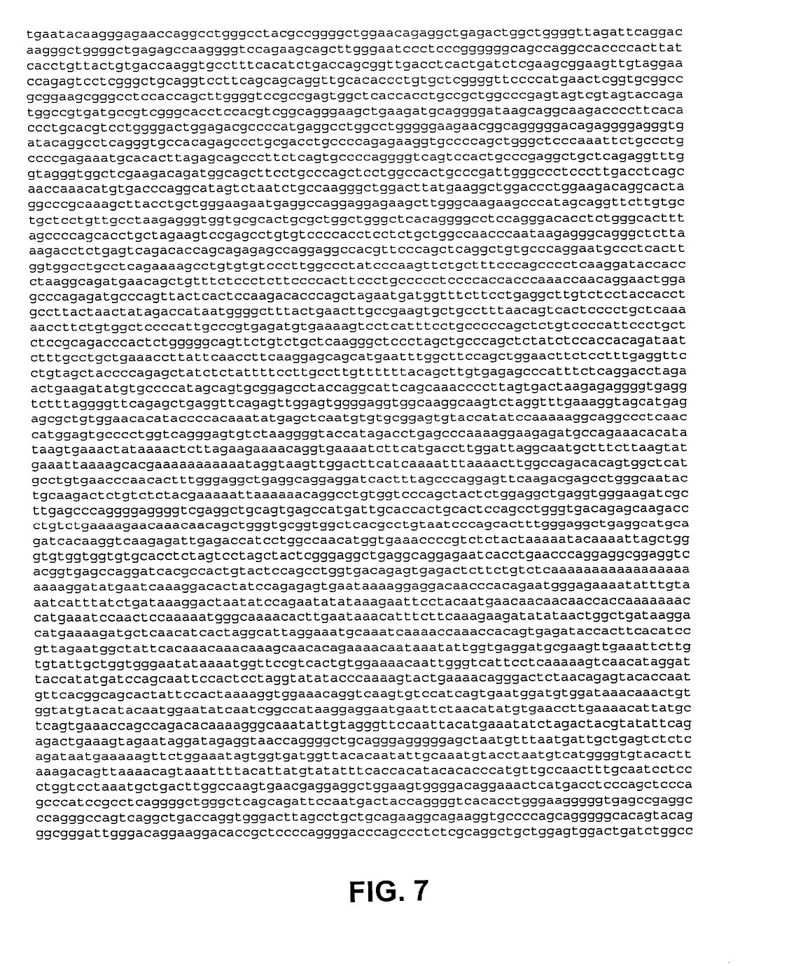

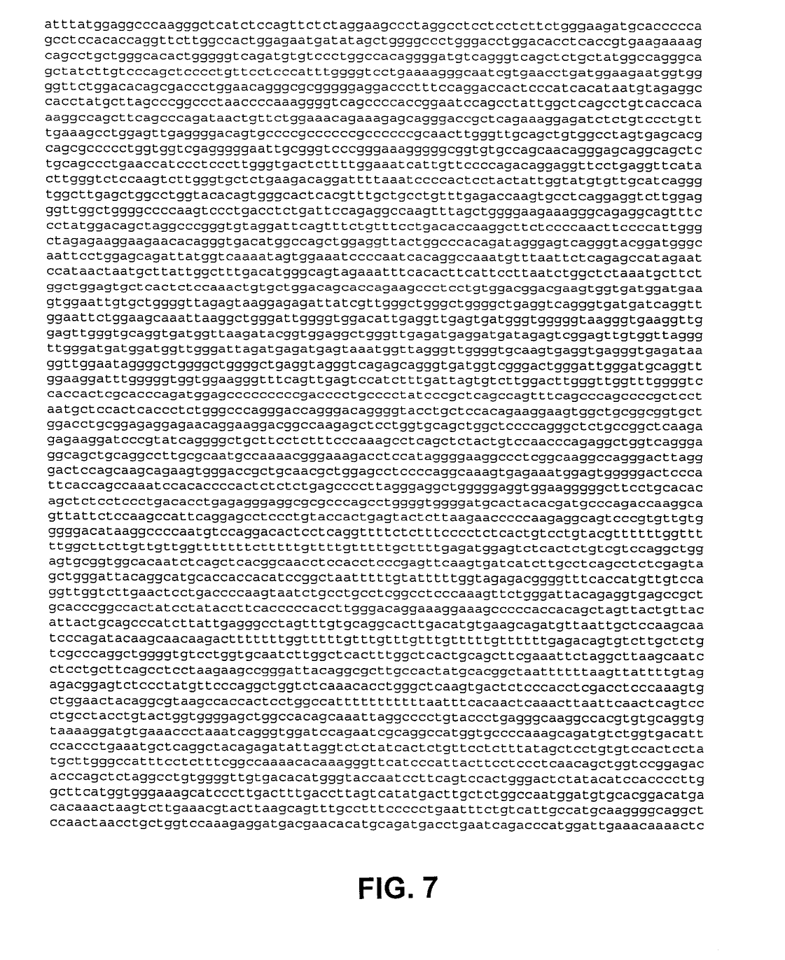

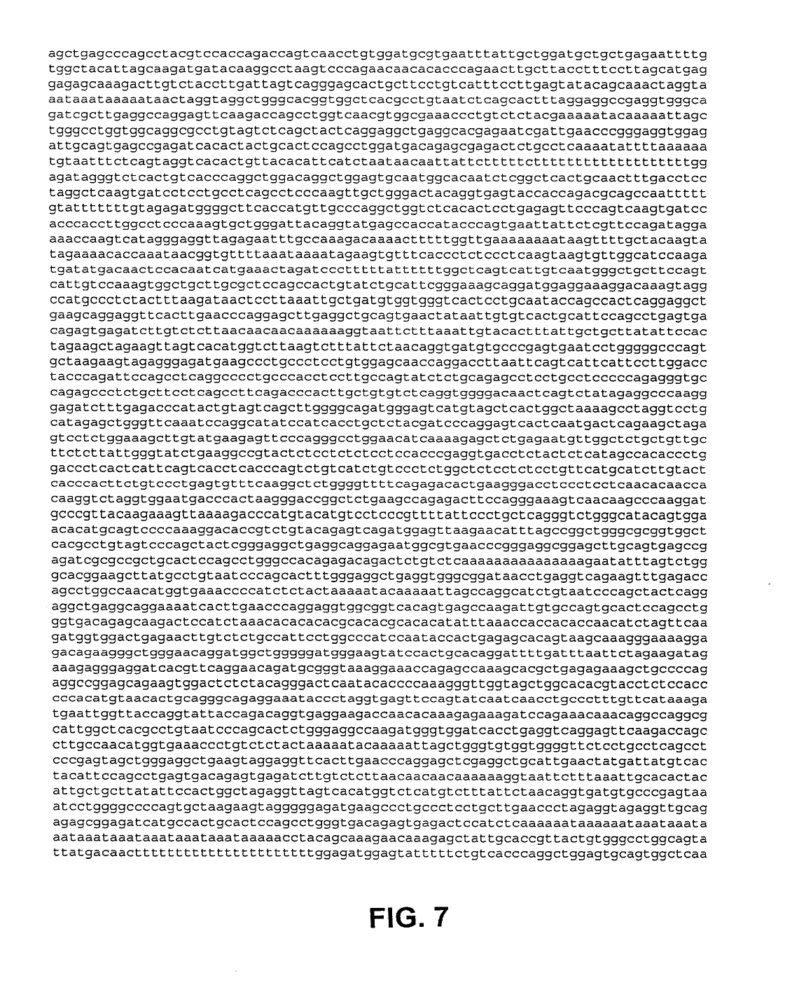

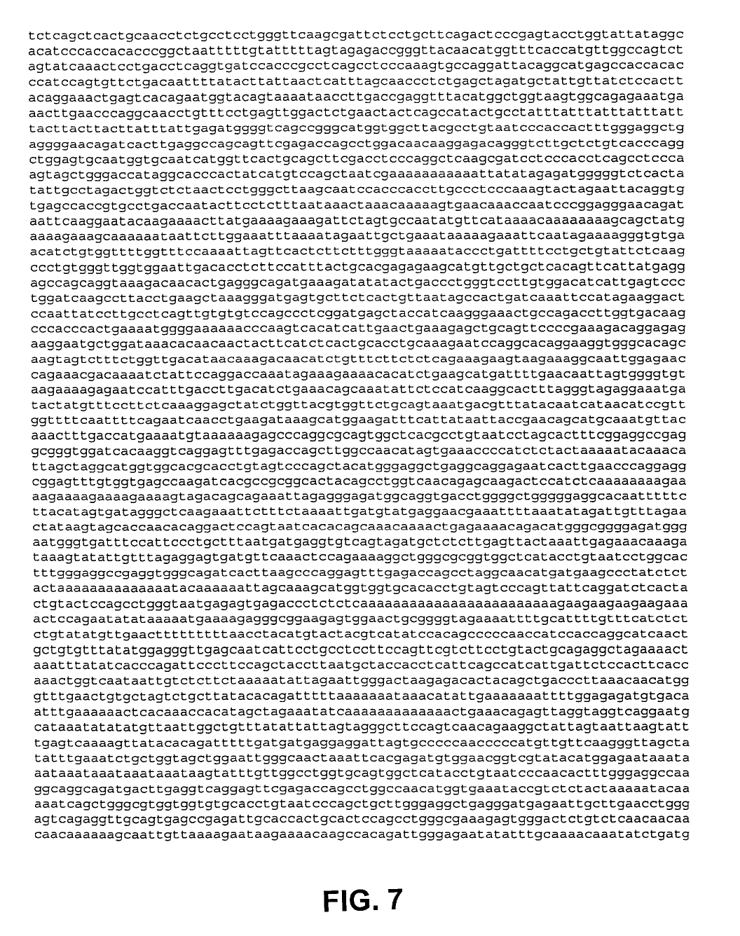

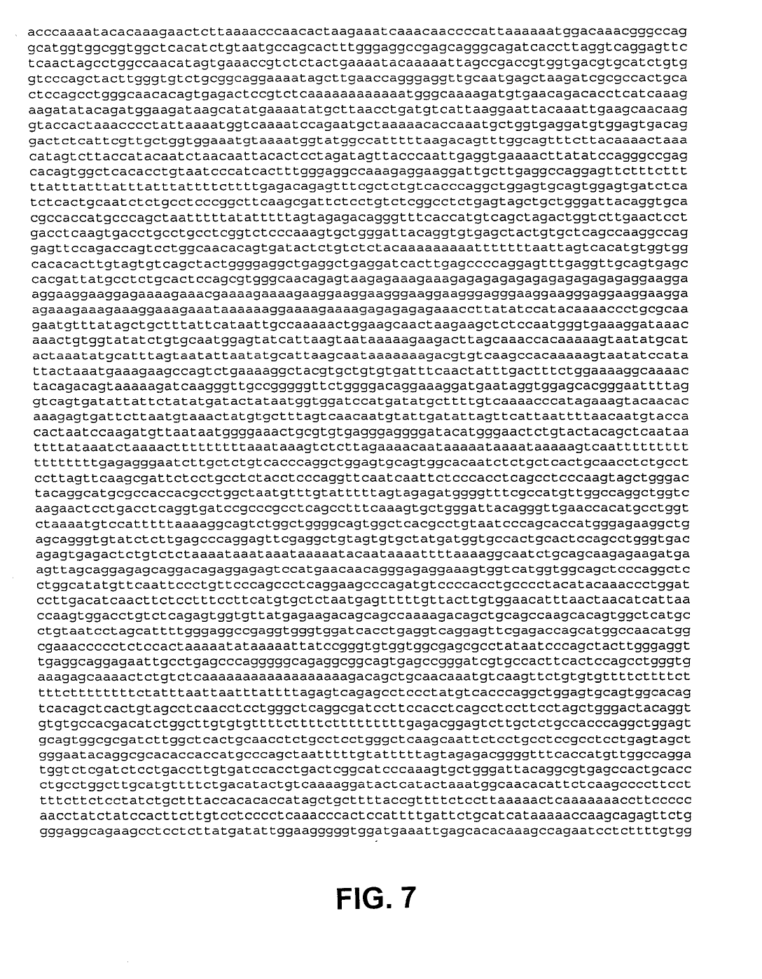

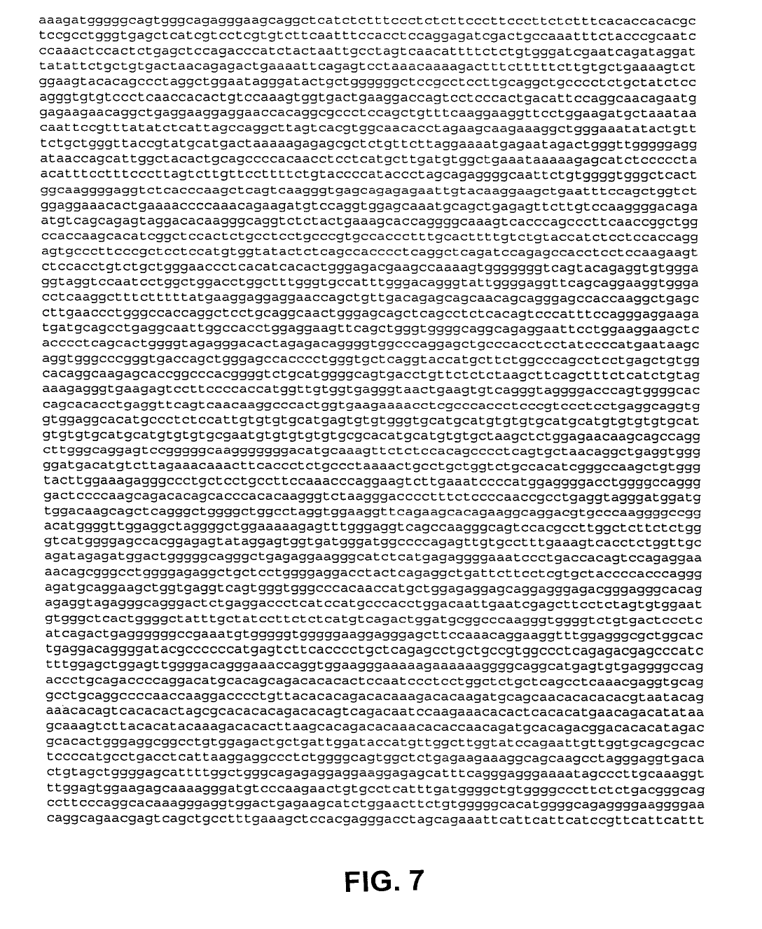

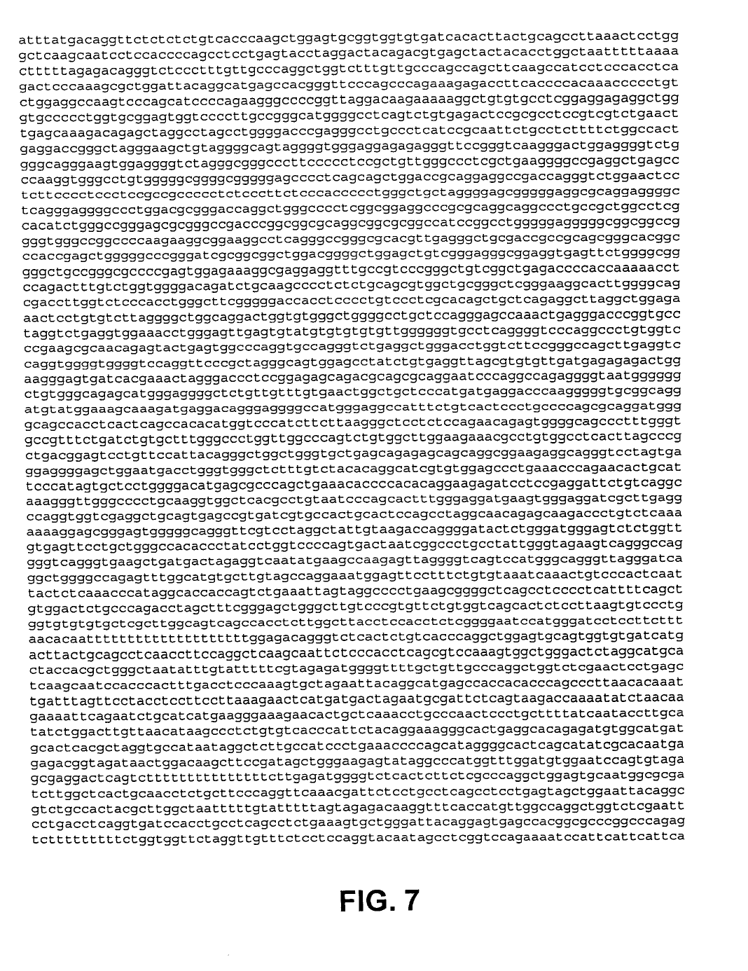

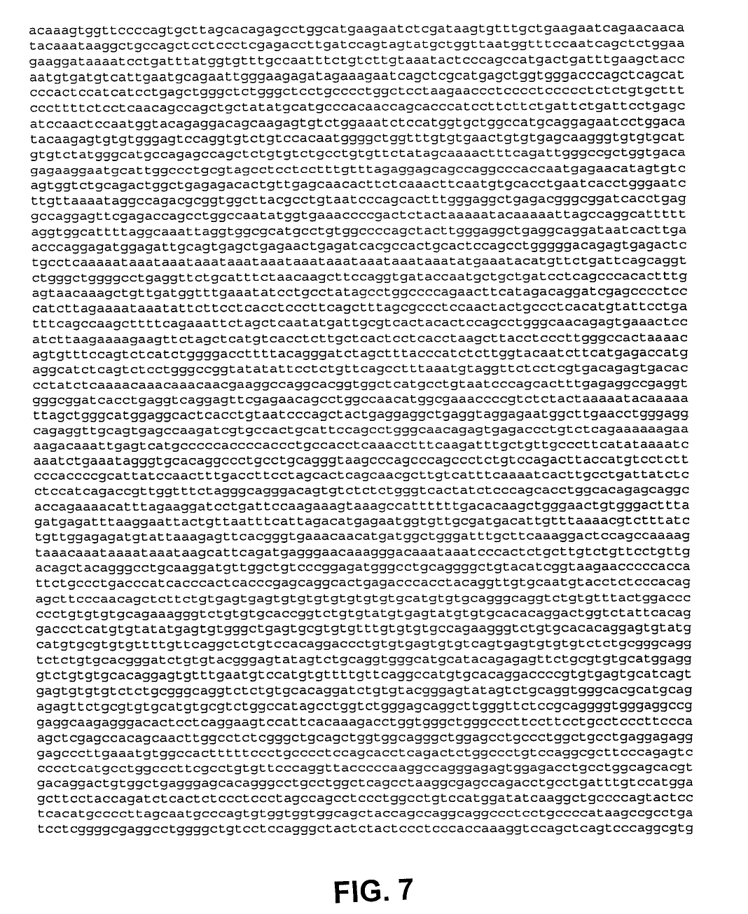

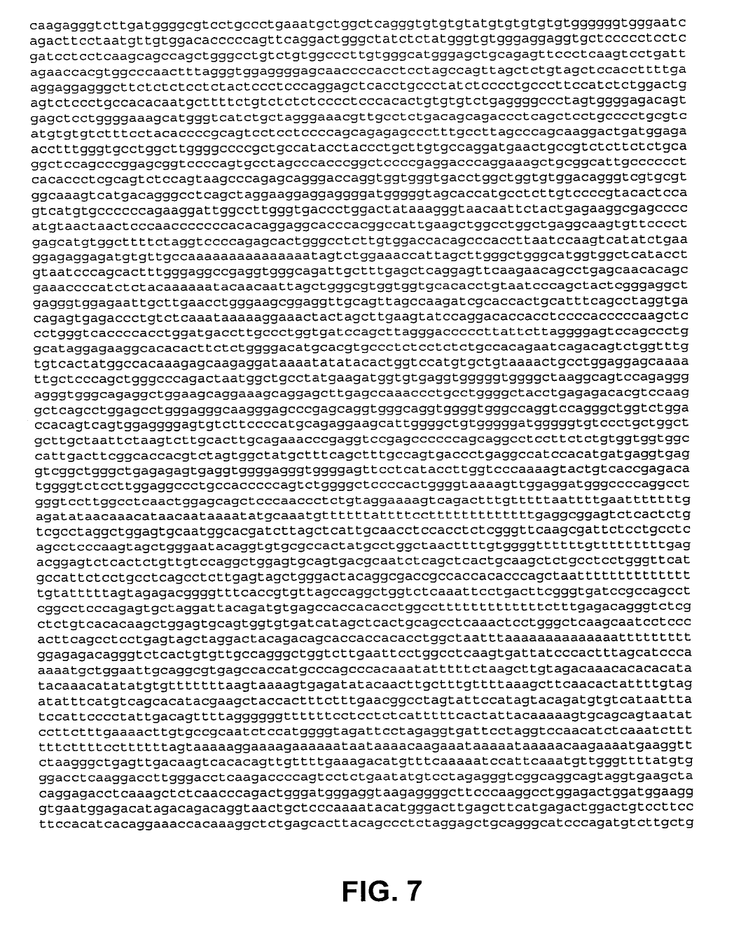

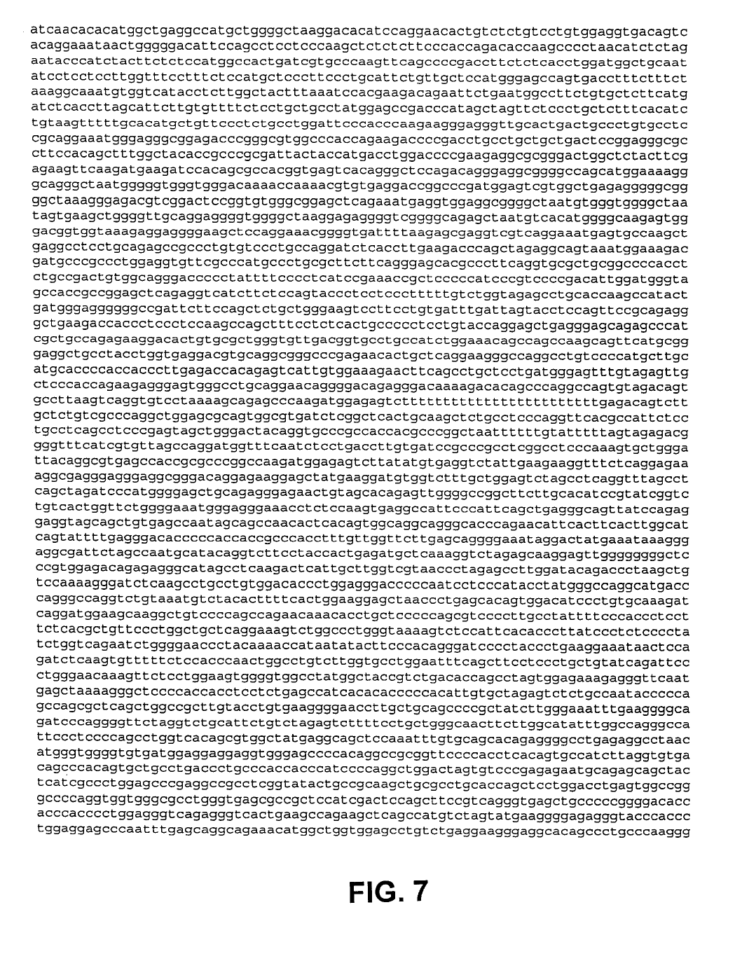

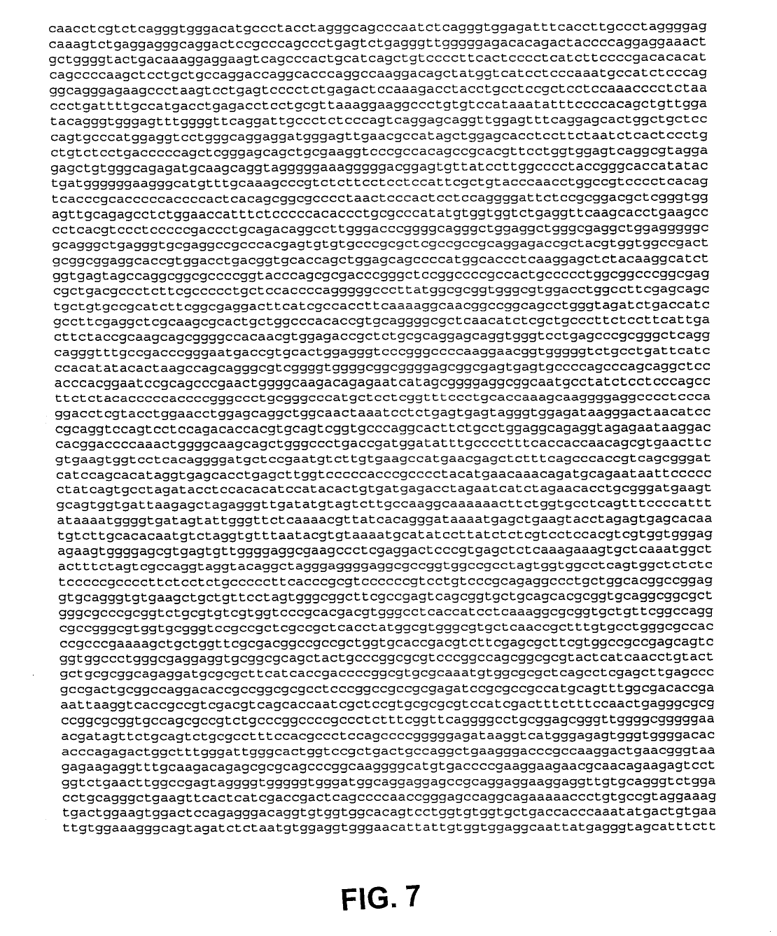

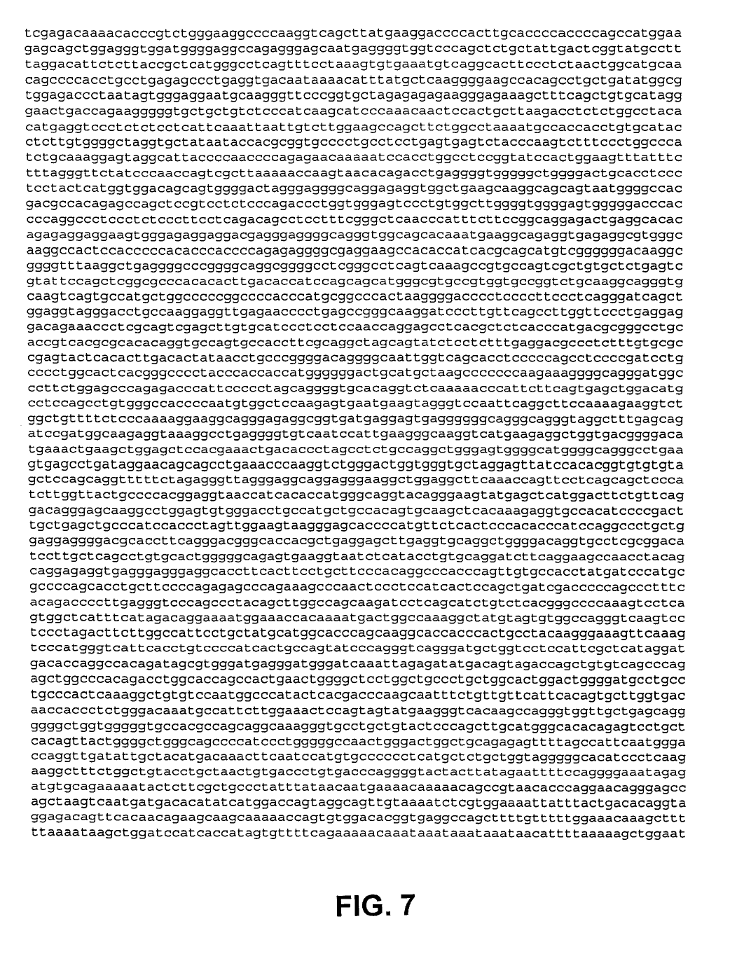

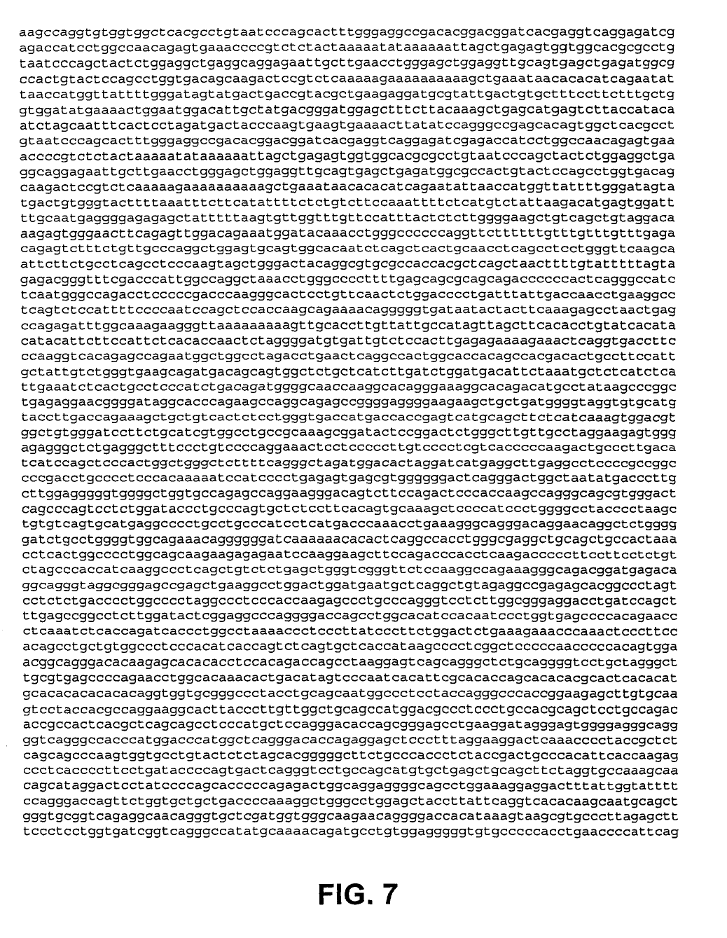

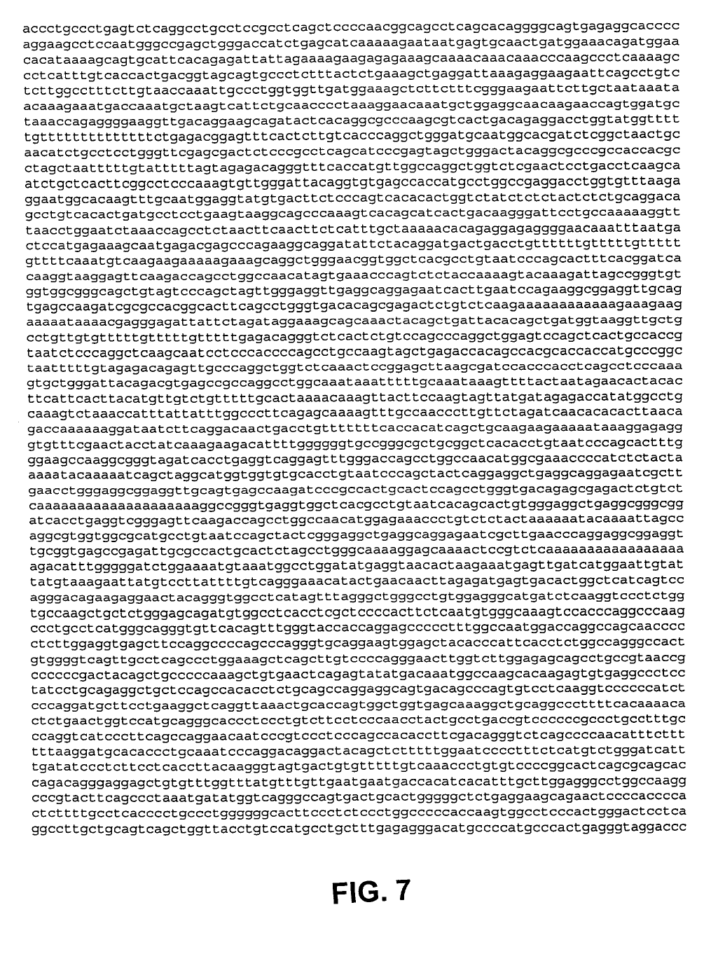

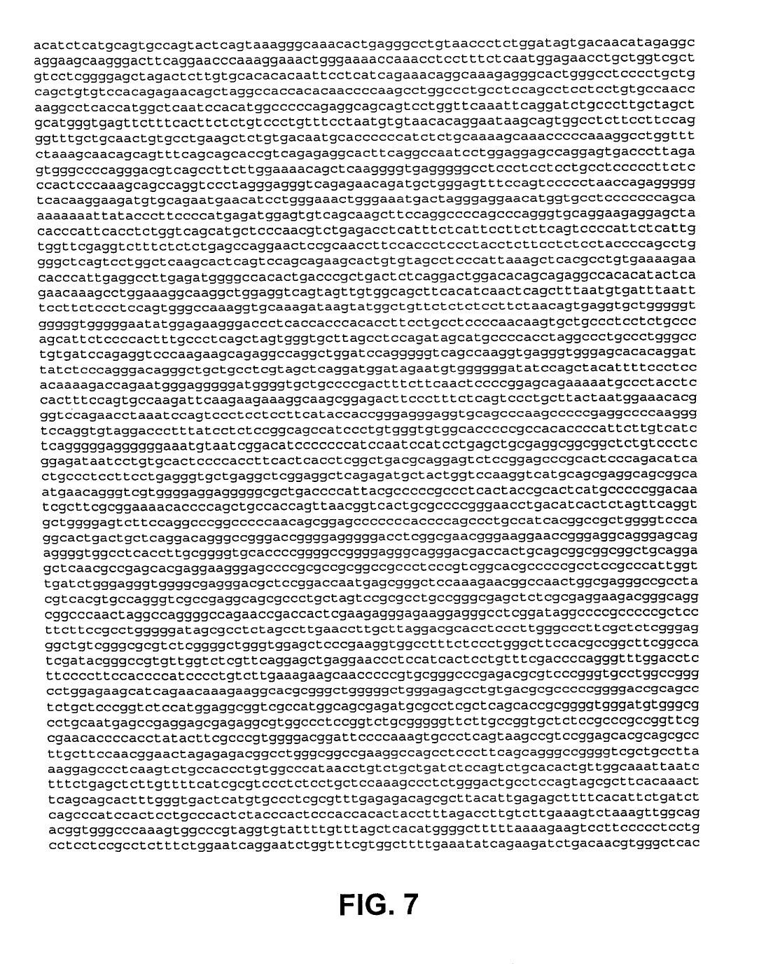

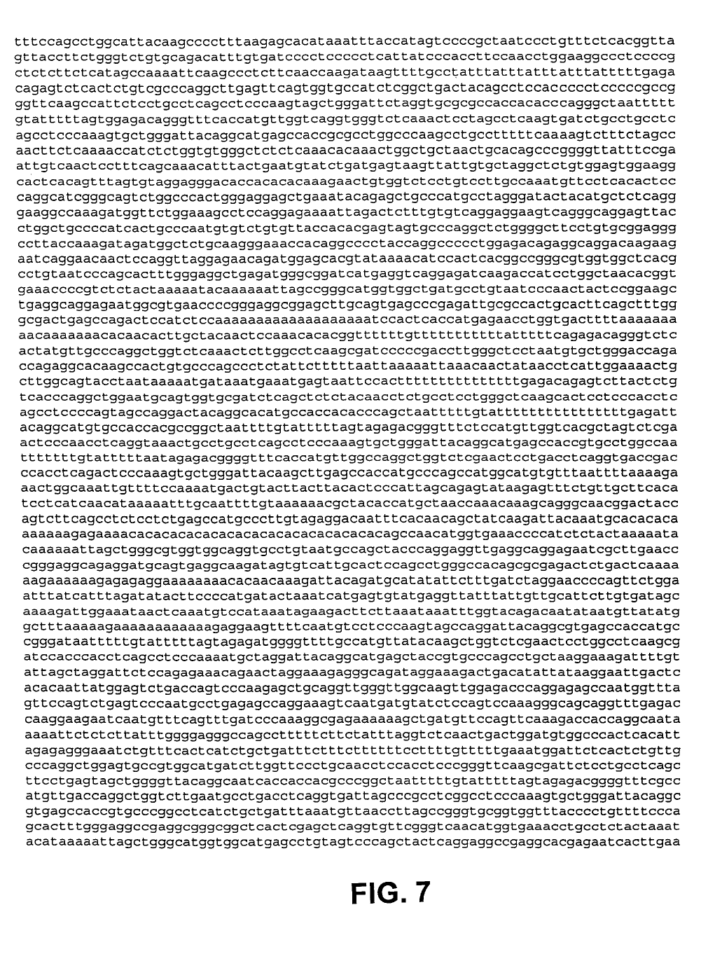

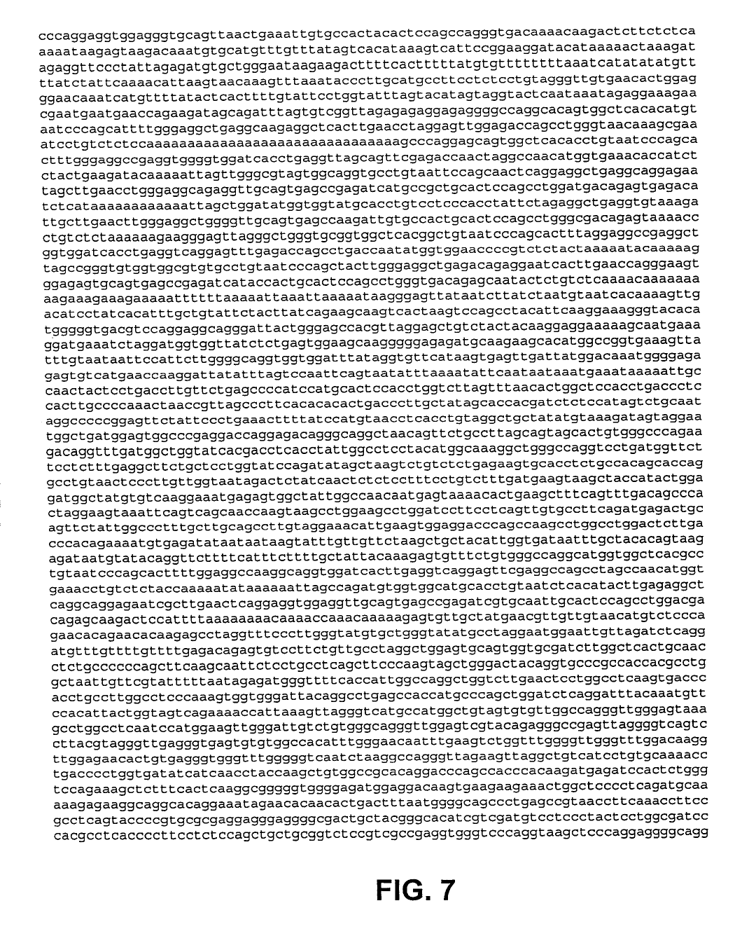

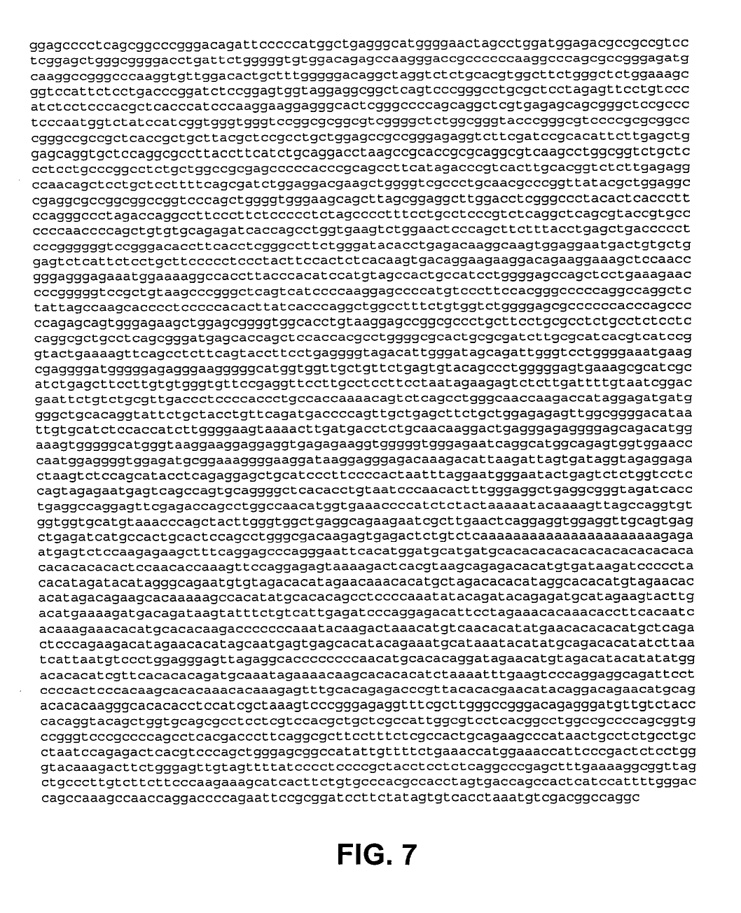

The invention also relates to isolated nucleic acids comprising regions required for accurate expression of Gene 216 (e.g., Gene 216 promoter (e.g., SEQ ID NO:8), enhancer (e.g., SEQ ID NO:7), and polyadenylation sequences). In a preferred embodiment, the present invention is directed to at least 15 contiguous nucleotides of the nucleic acid sequence of SEQ ID NO:1 or SEQ ID NO:6. More particularly, embodiments of this invention include the BAC clone containing segments of Gene 216 including RPCI_{—}1098L22 as set forth in SEQ ID NO:5 (FIG. 7).

The invention further relates to nucleic acids (e.g., DNA or RNA) that hybridize to a) a nucleic acid encoding a Gene 216 polypeptide, such as a nucleic acid having the sequence of SEQ ID NO:1 or SEQ ID NO:6; b) sequence-conservative, function-conservative, and non-conservative variants of (a); and c) fragments or portions of (a) or (b). Nucleic acids that hybridize to the sequence of SEQ ID NO:1 or SEQ ID NO:6 can be double- or single-stranded. Hybridization to the sequence of SEQ ID NO:1 or SEQ ID NO:6 includes hybridization to the strand shown or its complementary strand.

The present invention also relates to nucleic acids that encode a polypeptide having the amino acid sequence of SEQ ID NO:4 or SEQ ID NO:363, or functional equivalents thereof. A functional equivalent of a Gene 216 protein includes fragments or variants that perform at least on characteristic function of the Gene 216 protein (e.g., proteolysis, adhesion, fusion, antigenic, or intracellular activity). Preferably, a functional equivalent will share at least 65% sequence identity with the Gene 216 polypeptide.

In preferred embodiments, nucleic acids of the present invention share at least 50%, preferably at least 60-70%, more preferably at least 70-80% sequence identity, and even more preferably at least 90-100% sequence identity with the sequences of SEQ ID NO:1 or SEQ ID NO:6, or fragments or portions thereof. Sequence identity calculations can be performed using computer programs, hybridization methods, or calculations. Preferred computer program methods to determine identity and similarity between two sequences include, but are not limited to, the GCG program package, BLASTN, BLASTX, TBLASTX, and FASTA (J. Devereux et al., 1984, Nucleic Acids Research 12(1):387; S. F. Altschul et al., 1990, J. Molec. Biol. 215:403-410; W. Gish and D. J. States, 1994, Nature Genet. 3:266-272; W. R. Pearson and D. J. Lipman, 1988, Proc Natl. Acad. Sci. USA 85(8):2444-8). The BLAST programs are publicly available from NCBI and other sources. The well-known Smith Waterman algorithm may also be used to determine identity.

For example, nucleotide sequence identity can be determined by comparing a query sequences to sequences in publicly available sequence databases (NCBI) using the BLASTN2 algorithm (S. F. Altschul et al., 1997, Nucl. Acids Res., 25:3389-3402). The parameters for a typical search are: E=0.05, v=50, B=50, wherein E is the expected probability score cutoff, V is the number of database entries returned in the reporting of the results, and B is the number of sequence alignments returned in the reporting of the results (S. F. Altschul et al., 1990, J. Mol. Biol., 215:403-410).

In another approach, nucleotide sequence identity can be calculated using the following equation: % identity=(number of identical nucleotides)/(alignment length in nucleotides)* 100. For this calculation, alignment length includes internal gaps but not includes terminal gaps. Alternatively, nucleotide sequence identity can be determined experimentally using the specific hybridization conditions described below.

In accordance with the present invention, polynucleotide alterations are selected from the group consisting of at least one nucleotide deletion, substitution, including transition and transversion, insertion, or modification (e.g., via RNA or DNA analogs). Alterations may occur at the 5′ or 3′ terminal positions of the reference nucleotide sequence or anywhere between those terminal positions, interspersed either individually among the nucleotides in the reference sequence or in one or more contiguous groups within the reference sequence. Alterations of a polynucleotide sequence of SEQ ID NO:1 or SEQ ID NO:6 may create nonsense, missense, or frameshift mutations in this coding sequence, and thereby alter the polypeptide encoded by the polynucleotide following such alterations.

Such altered nucleic acids, including DNA or RNA, can be detected and isolated by hybridization under high stringency conditions or moderate stringency conditions, for example, which are chosen to prevent hybridization of nucleic acids having non-complementary sequences. “Stringency conditions” for hybridizations is a term of art which refers to the conditions of temperature and buffer concentration which permit hybridization of a particular nucleic acid to another nucleic acid in which the first nucleic acid may be perfectly complementary to the second, or the first and second may share some degree of complementarity which is less than perfect.

For example, certain high stringency conditions can be used which distinguish perfectly complementary nucleic acids from those of less complementarity. “High stringency conditions” and “moderate stringency conditions” for nucleic acid hybridizations are explained in F. M. Ausubel et al. (eds), 1995, Current Protocols in Molecular Biology, John Wiley and Sons, Inc., New York, N.Y., the teachings of which are hereby incorporated by reference. In particular, see pages 2.10.1-2.10.16 (especially pages 2.10.8-2.10.11) and pages 6.3.1-6.3.6. The exact conditions which determine the stringency of hybridization depend not only on ionic strength, temperature and the concentration of destabilizing agents such as formamide, but also on factors such as the length of the nucleic acid sequence, base composition, percent mismatch between hybridizing sequences and the frequency of occurrence of subsets of that sequence within other non-identical sequences. Thus, high or moderate stringency conditions can be determined empirically.

By varying hybridization conditions from a level of stringency at which no hybridization occurs to a level at which hybridization is first observed, conditions which will allow a given sequence to hybridize with the most similar sequences in the sample can be determined. Preferably the hybridizing sequences will have 60-70% sequence identity, more preferably 70-85% sequence identity, and even more preferably 90-100% sequence identity.

Typically, the hybridization reaction is initially performed under conditions of low stringency, followed by washes of varying, but higher stringency. Reference to hybridization stringency, e.g., high, moderate, or low stringency, typically relates to such washing conditions. Hybridization conditions are based on the melting temperature (Tₘ) of the nucleic acid probe or primer and are typically classified by degree of stringency of the conditions under which hybridization is measured (Ausubel et al., 1995). For example, high stringency hybridization typically occurs at about 5-10% C below the Tₘ; moderate stringency hybridization occurs at about 10-20% below the Tₘ; and low stringency hybridization occurs at about 20-25% below the Tₘ. The melting temperature can be approximated by the formulas as known in the art, depending on a number of parameters, such as the length of the hybrid or probe in number of nucleotides, or hybridization buffer ingredients and conditions. As a general guide, Tm decreases approximately 1° C. with every 1% decrease in sequence identity at any given SSC concentration. Generally, doubling the concentration of SSC results in an increase in Tₘ of ˜17° C. Using these guidelines, the washing temperature can be determined empirically for moderate or low stringency, depending on the level of mismatch sought.

High stringency hybridization conditions are typically carried out at 65 to 68° C. in 0.1× SSC and 0.1% SDS. Highly stringent conditions allow hybridization of nucleic acid molecules having about 95 to 100% sequence identity. Moderate stringency hybridization conditions are typically carried out at 50 to 65° C. in 1× SSC and 0.1% SDS. Moderate stringency conditions allow hybridization of sequences having at least about 80 to 95% nucleotide sequence identity. Low stringency hybridization conditions are typically carried out at 40 to 50° C. in 6× SSC and 0.1% SDS. Low stringency hybridization conditions allow detection of specific hybridization of nucleic acid molecules having at least about 50 to 80% nucleotide sequence identity.

For example, high stringency conditions can be attained by hybridization in 50% formamide, 5× Denhardt's solution, 5× SSPE or SSC (1× SSPE buffer comprises 0.15 M NaCl, 10 mM Na₂HPO₄, 1 mM EDTA; 1× SSC buffer comprises 150 mM NaCl, 15 mM sodium citrate, pH 7.0), 0.2% SDS at about 42° C., followed by washing in 1× SSPE or SSC and 0.1% SDS at a temperature of at least about 42° C., preferably about 55° C., more preferably about 65° C. Moderate stringency conditions can be attained, for example, by hybridization in 50% formamide, 5× Denhardt's solution, 5× SSPE or SSC, and 0.2% SDS at 42° C. to about 50° C., followed by washing in 0.2× SSPE or SSC and 0.2% SDS at a temperature of at least about 42° C., preferably about 55° C., more preferably about 65° C. Low stringency conditions can be attained, for example, by hybridization in 10% formamide, 5× Denhardt's solution, 6× SSPE or SSC, and 0.2% SDS at 42° C., followed by washing in 1× SSPE or SSC, and 0.2% SDS at a temperature of about 45° C., preferably about 50° C. in 4× SSC at 60° C. for 30 min.

High stringency hybridization procedures typically (1) employ low ionic strength and high temperature for washing, such as 0.015 M NaCl/0.0015 M sodium citrate, pH 7.0 (0.1× SSC) with 0.1% sodium dodecyl sulfate (SDS) at 50° C.; (2) employ during hybridization 50% (vol/vol) formamide with 5× Denhardt's solution (0.1% weight/volume highly purified bovine serum albumin/0.1% wt/vol Ficoll/0.1% wt/vol polyvinylpyrrolidone), 50 mM sodium phosphate buffer at pH 6.5 and 5× SSC at 42° C.; or (3) employ hybridization with 50% formamide, 5× SSC, 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC and 0.1% SDS.

In one particular embodiment, high stringency hybridization conditions may be attained by:

Prehybridization treatment of the support (e.g. nitrocellulose filter or nylon membrane), to which is bound the nucleic acid capable of hybridizing with any of the sequences of the invention, is carried out at 65° C. for 6 hr with a solution having the following composition: 4× SSC, 10× Denhardt's (1× Denhardt's comprises 1% Ficoll, 1% polyvinylpyrrolidone, 1% BSA (bovine serum albumin); 1× SSC comprises of 0.15 M of NaCl and 0.015 M of sodium citrate, pH 7);

Replacement of the pre-hybridization solution in contact with the support by a buffer solution having the following composition: 4× SSC, 1× Denhardt's, 25 mM NaPO₄, pH 7, 2 mM EDTA, 0.5% SDS, 100 μg/ml of sonicated salmon sperm DNA containing a nucleic acid derived from the sequences of the invention as probe, in particular a radioactive probe, and previously denatured by a treatment at 100° C. for 3 min;

Incubation for 12 hr at 65° C.; —

Successive washings with the following solutions: 1) four washings with 2× SSC, 1× Denhardt's, 0.5% SDS for 45 min at 65° C.; 2) two washings with 0.2× SSC, 0.1× SSC for 45 min at 65° C.; and 3) 0.1× SSC, 0.1% SDS for 45 min at 65° C.

Additional examples of high, medium, and low stringency conditions can be found in Sambrook et al., 1989. Exemplary conditions are also described in M. H. Krause and S. A. Aaronson, 1991, Methods in Enzymology, 200:546-556; Ausubel et al., 1995. It is to be understood that the low, moderate and high stringency hybridization/washing conditions may be varied using a variety of ingredients, buffers, and temperatures well known to and practiced by the skilled practitioner.

Isolated nucleic acids that are characterized by their ability to hybridize to (a) a nucleic acid encoding a Gene 216 polypeptide, such as the nucleic acids depicted as SEQ ID NO:1 or SEQ ID NO:6, b) the complement of (a), (c) or a portion of (a) or (b) (e.g., under high or moderate stringency conditions), may further encode a protein or polypeptide having at least one function characteristic of a Gene 216 polypeptide, such as proteolysis, adhesion, fusion, and intracellular activity, or binding of antibodies that also bind to non-recombinant Gene 216 protein or polypeptide. The catalytic or binding function of a protein or polypeptide encoded by the hybridizing nucleic acid may be detected by standard enzymatic assays for activity or binding (e.g., assays that measure the binding of a transit peptide or a precursor, or other components of the translocation machinery). Enzymatic assays, complementation tests, or other suitable methods can also be used in procedures for the identification and/or isolation of nucleic acids which encode a polypeptide having the amino acid sequence of SEQ ID NO:4 or SEQ ID NO:363, or a functional equivalent of this polypeptide. The antigenic properties of proteins or polypeptides encoded by hybridizing nucleic acids can be determined by immunological methods employing antibodies that bind to a Gene 216 polypeptide such as immunoblot, immunoprecipitation and radioimmunoassay. PCR methodology, including RAGE (Rapid Amplification of Genomic DNA Ends), can also be used to screen for and detect the presence of nucleic acids which encode Gene 216-like proteins and polypeptides, and to assist in cloning such nucleic acids from genomic DNA. PCR methods for these purposes can be found in M. A. Innis et al., 1990, PCR Protocols: A Guide to Methods and Applications, Academic Press, Inc., San Diego, Calif., incorporated herein by reference.

It is understood that, as a result of the degeneracy of the genetic code, many nucleic acid sequences are possible which encode a Gene 216-like protein or polypeptide. Some of these will share little identity to the nucleotide sequences of any known or naturally-occurring Gene 216-like gene but can be used to produce the proteins and polypeptides of this invention by selection of combinations of nucleotide triplets based on codon choices. Such variants, while not hybridizable to a naturally-occurring Gene 216 gene under conditions of high stringency, are contemplated within this invention.

Also encompassed by the present invention are alternate splice variants produced by differential processing of the primary transcript(s) from Gene 216 genomic DNA. An alternate splice variant may comprise, for example, the sequence of any one of SEQ ID NO:2 and SEQ ID NO:350-362. Alternate splice variants can also comprise other combinations of introns/exons of SEQ ID NO:1 or SEQ ID NO:6, which can be determined by those of skill in the art. Alternate splice variants can be determined experimentally, for example, by isolating and analyzing cellular RNAs (e.g., Southern blotting or PCR), or by screening cDNA libraries using the Gene 216 nucleic acid probes or primers described herein. In another approach, alternate splice variants can be predicted using various methods, computer programs, or computer systems available to practitioners in the field.

General methods for splice site prediction can be found in Nakata, 1985, Nucleic Acids Res. 13:5327-5340. In addition, splice sites can be predicted using, for example, the GRAIL™ (E. C. Uberbacher and R. J. Mural, 1991, Proc. Natl. Acad. Sci. USA, 88:11261-11265; E. C. Uberbacher, 1995, Trends Biotech., 13:497-500; http://grail.lsd.ornl.gov/grailexp); GenView (L. Milanesi et al., 1993, Proceedings of the Second International Conference on Bioinformatics, Supercomputing, and Complex Genome Analysis, H. A. Lim et al. (eds), World Scientific Publishing, Singapore, pp. 573-588; http://l25.1tba.mi.cnr.it/˜webgene/wwwgene_help.html); SpliceView (http://www. itba.mi.cnr.it/webgene); and HSPL (V.V. Solovyev et al., 1994, Nucleic Acids Res. 22:5156-5163; V. V. Solovyev et al., 1994, “The Prediction of Human Exons by Oligonucleotide Composition and Discriminant Analysis of Spliceable Open Reading Frames,” R. Altman et al. (eds), The Second International conference on Intelligent systems for Molecular Biology, AAAI Press, Menlo Park, Calif., pp. 354-362; V. V. Solovyev et al., 1993, “Identification Of Human Gene Functional Regions Based On Oligonucleotide Composition,” L. Hunter et al. (eds), In Proceedings of First International conference on Intelligent System for Molecular Biology, Bethesda, pp. 371-379) computer systems.

Additionally, computer programs such as GeneParser (E. E. Snyder and G. D. Stormo, 1995, J. Mol. Biol. 248: 1-18; E. E. Snyder and G. D. Stormo, 1993, Nucl. Acids Res. 21(3): 607-613; http://mcdb.colorado.edu/˜eesnyder/GeneParser.html); MZEF (M. Q. Zhang, 1997, Proc. Natl. Acad. Sci. USA, 94:565-568; http://fargon.cshl.org/genefinder); MORGAN (S. Salzberg et al., 1998, J. Comp. Biol. 5:667-680; S. Salzberg et al. (eds), 1998, Computational Methods in Molecular Biology, Elsevier Science, New York, N.Y., pp. 187-203); VEIL (J. Henderson et al., 1997, J. Comp. Biol. 4:127-141); GeneScan (S. Tiwari et al., 1997, CABIOS (BioInformatics) 13: 263-270); GeneBuilder (L. Milanesi et al., 1999, Bioinformatics 15:612-621); Eukaryotic GeneMark (J. Besemer et al., 1999, Nucl. Acids Res. 27:3911-3920); and FEXH (V. V. Solovyev et al., 1994, Nucleic Acids Res. 22:5156-5163). In addition, splice sites (i.e., former or potential splice sites) in cDNA sequences can be predicted using, for example, the RNASPL (V. V. Solovyev et al., 1994, Nucleic Acids Res. 22:5156-5163); or INTRON (A. Globek et al., 1991, INTRON version 1.1 manual, Laboratory of Biochemical Genetics, NIMH, Washington, D.C.) programs.

The present invention also encompasses naturally-occurring polymorphisms of Gene 216. As will be understood by those in the art, the genomes of all organisms undergo spontaneous mutation in the course of their continuing evolution generating variant forms of gene sequences (Gusella, 1986, Ann. Rev. Biochem. 55:831-854). Restriction fragment length polymorphisms (RFLPs) include variations in DNA sequences that alter the length of a restriction fragment in the sequence (Botstein et al., 1980, Am. J. Hum. Genet. 32, 314-331 (1980). RFLPs have been widely used in human and animal genetic analyses (see WO 90/13668; WO90/11369; Donis-Keller, 1987, Cell 51:319-337; Lander et al., 1989, Genetics 121: 85-99). Short tandem repeats (STRs) include tandem di-, tri- and tetranucleotide repeated motifs, also termed variable number tandem repeat (VNTR) polymorphisms. VNTRs have been used in identity and paternity analysis (U.S. Pat. No. 5,075,217; Armour et al., 1992, FEBS Lett. 307:113-115; Horn et al., WO 91/14003; Jeffreys, EP 370,719), and in a large number of genetic mapping studies.

Single nucleotide polymorphisms (SNPs) are far more frequent than RFLPS, STRs, and VNTRs. SNPs may occur in protein coding (e.g., exon), or non-coding (e.g., intron, 5′UTR, 3′UTR) sequences. SNPs in protein coding regions may comprise silent mutations that do not alter the amino acid sequence of a protein. Alternatively, SNPs in protein coding regions may produce conservative or non-conservative amino acid changes, described in detail below. In some cases, SNPs may give rise to the expression of a defective or other variant protein and, potentially, a genetic disease. SNPs within protein-coding sequences can give rise to genetic diseases, for example, in the β-globin (sickle cell anemia) and CFTR (cystic fibrosis) genes. In non-coding sequences, SNPs may also result in defective protein expression (e.g., as a result of defective splicing). Other single nucleotide polymorphisms have no phenotypic effects.

Single nucleotide polymorphisms can be used in the same manner as RFLPs and VNTRs, but offer several advantages. Single nucleotide polymorphisms tend to occur with greater frequency and are typically spaced more uniformly throughout the genome than other polymorphisms. Also, different SNPs are often easier to distinguish than other types of polymorphisms (e.g., by use of assays employing allele-specific hybridization probes or primers). In one embodiment of the present invention, a Gene 216 nucleic acid contains at least one SNP as set forth in Table 10, herein below. Various combinations of these SNPs are also encompassed by the invention. In a preferred aspect, a Gene 216 SNP is associated with a lung-related disorder, such as asthma.

The nucleic acid sequences of the present invention may be derived from a variety of sources including DNA, cDNA, synthetic DNA, synthetic RNA, or combinations thereof. Such sequences may comprise genomic DNA, which may or may not include naturally occurring introns. Moreover, such genomic DNA may be obtained in association with promoter regions or poly (A) sequences. The sequences, genomic DNA, or cDNA may be obtained in any of several ways. Genomic DNA can be extracted and purified from suitable cells by means well known in the art. Alternatively, mRNA can be isolated from a cell and used to produce cDNA by reverse transcription or other means.

The nucleic acids described herein are used in the methods of the present invention for production of proteins or polypeptides, through incorporation into cells, tissues, or organisms. In one embodiment, DNA containing all or part of the coding sequence for a Gene 216 polypeptide, or DNA which hybridizes to DNA having the sequence SEQ ID NO:1 or SEQ ID NO:6, is incorporated into a vector for expression of the encoded polypeptide in suitable host cells. The encoded polypeptide consisting of Gene 216, or its functional equivalent is capable of normal activity, such as proteolysis, adhesion, fusion, and intracellular activity.

The invention also concerns the use of the nucleotide sequence of the nucleic acids of this invention to identify DNA probes for Gene 216 genes, PCR primers to amplify Gene 216 genes, nucleotide polymorphisms in Gene 216 genes, and regulatory elements of the Gene 216 genes.

The nucleic acids of the present invention find use as primers and templates for the recombinant production of disorder-associated peptides or polypeptides, for chromosome and gene mapping, to provide antisense sequences, for tissue distribution studies, to locate and obtain full length genes, to identify and obtain homologous sequences (wild-type and mutants), and in diagnostic applications.

Probes may also be used for the detection of Gene 216-related sequences, and should preferably contain at least 50%, preferably at least 80%, identity to Gene 216 polynucleotide, or a complementary sequence, or fragments thereof. The probes of this invention may be DNA or RNA, the probes may comprise all or a portion of the nucleotide sequence of SEQ ID NO:1 or SEQ ID NO:6, or a complementary sequence thereof, and may include promoter, enhancer elements, and introns of the naturally occurring Gene 216 polynucleotide.

The probes and primers based on the Gene 216 gene sequences disclosed herein are used to identify homologous Gene 216 gene sequences and proteins in other species. These Gene 216 gene sequences and proteins are used in the diagnostic/prognostic, therapeutic and drug-screening methods described herein for the species from which they have been isolated.

Vectors and Host Cells

The invention also provides vectors comprising the disorder-associated sequences, or derivatives or fragments thereof, and host cells for the production of purified proteins. A large number of vectors, including bacterial, yeast, and mammalian vectors, have been described for replication and/or expression in various host cells or cell-free systems, and may be used for gene therapy as well as for simple cloning or protein expression.

In one aspect, an expression vectors comprises a nucleic acid encoding a Gene 216 polypeptide or peptide, as described herein, operably linked to at least one regulatory sequence. Regulatory sequences are known in the art and are selected to direct expression of the desired protein in an appropriate host cell. Accordingly, the term regulatory sequence includes promoters, enhancers and other expression control elements (see D. V. Goeddel (1990) Methods Enzymol. 185:3-7). Enhancer and other expression control sequences are described in Enhancers and Eukaryotic Gene Expression, Cold Spring Harbor Press, Cold Spring Harbor, N.Y. (1983). It should be understood that the design of the expression vector may depend on such factors as the choice of the host cell to be transfected and/or the type of polypeptide desired to be expressed.

Several regulatory elements (e.g., promoters) have been isolated and shown to be effective in the transcription and translation of heterologous proteins in the various hosts. Such regulatory regions, methods of isolation, manner of manipulation, etc. are known in the art. Non-limiting examples of bacterial promoters include the β-lactamase (penicillinase) promoter; lactose promoter; tryptophan (trp) promoter; araBAD (arabinose) operon promoter; lambda-derived P₁ promoter and N gene ribosome binding site; and the hybrid tac promoter derived from sequences of the trp and lac UV5 promoters. Non-limiting examples of yeast promoters include the 3-phosphoglycerate kinase promoter, glyceraldehyde-3-phosphate dehydrogenase (GAPDH) promoter, galactokinase (GAL1) promoter, galactoepimerase promoter, and alcohol dehydrogenase (ADH1) promoter. Suitable promoters for mammalian cells include, without limitation, viral promoters, such as those from Simian Virus 40 (SV40), Rous sarcoma virus (RSV), adenovirus (ADV), and bovine papilloma virus (BPV). Preferred replication and inheritance systems include M13, ColE1, SV40, baculovirus, lambda, adenovirus, CEN ARS, 2 μm ARS and the like. While expression vectors may replicate autonomously, they may also replicate by being inserted into the genome of the host cell, by methods well known in the art.

To obtain expression in eukaryotic cells, terminator sequences, polyadenylation sequences, and enhancer sequences that modulate gene expression may be required. Sequences that cause amplification of the gene may also be desirable. These sequences are well known in the art. Furthermore, sequences that facilitate secretion of the recombinant product from cells, including, but not limited to, bacteria, yeast, and animal cells, such as secretory signal sequences and/or preprotein or proprotein sequences, may also be included. Such sequences are well described in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that 1) confer resistance to antibiotics or other toxic substances, e.g. ampicillin, neomycin, methotrexate, etc.; 2) complement auxotrophic deficiencies, or 3) supply critical nutrients not available from complex media, e.g., the gene encoding D-alanine racemase for Bacilli. Markers may be an inducible or non-inducible gene and will generally allow for positive selection. Non-limiting examples of markers include the ampicillin resistance marker (i.e., beta-lactamase), tetracycline resistance marker, neomycin/kanamycin resistance marker (i.e., neomycin phosphotransferase), dihydrofolate reductase, glutamine synthetase, and the like. The choice of the proper selectable marker will depend on the host cell, and appropriate markers for different hosts as understood by those of skill in the art.

Suitable expression vectors for use with the present invention include, but are not limited to, pUC, pBluescript (Stratagene), pET (Novagen, Inc., Madison, Wis.), and pREP (Invitrogen) plasmids. Vectors can contain one or more replication and inheritance systems for cloning or expression, one or more markers for selection in the host, e.g. antibiotic resistance, and one or more expression cassettes. The inserted coding sequences can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

Suitable cell-free expression systems for use with the present invention include, without limitation, rabbit reticulocyte lysate, wheat germ extract, canine pancreatic microsomal membranes, E. coli S30 extract, and coupled transcription/translation systems (Promega Corp., Madison, Wis.). These systems allow the expression of recombinant polypeptides or peptides upon the addition of cloning vectors, DNA fragments, or RNA sequences containing protein-coding regions and appropriate promoter elements.

Non-limiting examples of suitable host cells include bacteria, archea, insect, fungi (e.g., yeast), plant, and animal cells (e.g., mammalian, especially human). Of particular interest are Escherichia coli, Bacillus subtilis, Saccharomyces cerevisiae, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, NY). Examples of commonly used mammalian host cell lines are VERO and HeLa cells, CHO cells, and WI38, BHK, and COS cell lines, although it will be appreciated by the skilled practitioner that other cell lines may be used, e.g., to provide higher expression desirable glycosylation patterns, or other features.

Host cells can be transformed, transfected, or infected as appropriate by any suitable method including electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Alternatively, vectors containing the nucleic acids of interest can be transcribed in vitro, and the resulting RNA introduced into the host cell by well-known methods, e.g., by injection (see, Kubo et al., 1988, FEBS Letts. 241:119). The cells into which have been introduced nucleic acids described above are meant to also include the progeny of such cells.

The nucleic acids of the invention may be isolated directly from cells. Alternatively, the polymerase chain reaction (PCR) method can be used to produce the nucleic acids of the invention, using either RNA (e.g., mRNA) or DNA (e.g., genomic DNA) as templates. Primers used for PCR can be synthesized using the sequence information provided herein and can further be designed to introduce appropriate new restriction sites, if desirable, to facilitate incorporation into a given vector for recombinant expression.

Using the information provided in SEQ ID NO:1 and SEQ ID NO:6, one skilled in the art will be able to clone and sequence all representative nucleic acids of interest, including nucleic acids encoding complete protein-coding sequences. It is to be understood that non-protein-coding sequences contained within SEQ ID NO:1 and SEQ ID NO:3 and the genomic sequences of SEQ ID NO:6 and SEQ ID NO:5 are also within the scope of the invention. Such sequences include, without limitation, sequences important for replication, recombination, transcription, and translation. Non-limiting examples include promoters and regulatory binding sites involved in regulation of gene expression, and 5′- and 3′- untranslated sequences (e.g., ribosome-binding sites) that form part of mRNA molecules.

The nucleic acids of this invention can be produced in large quantities by replication in a suitable host cell. Natural or synthetic nucleic acid fragments, comprising at least ten contiguous bases coding for a desired peptide or polypeptide can be incorporated into recombinant nucleic acid constructs, usually DNA constructs, capable of introduction into and replication in a prokaryotic or eukaryotic cell. Usually the nucleic acid constructs will be suitable for replication in a unicellular host, such as yeast or bacteria, but may also be intended for introduction to (with and without integration within the genome) cultured mammalian or plant or other eukaryotic cells, cell lines, tissues, or organisms. The purification of nucleic acids produced by the methods of the present invention is described, for example, in Sambrook et al., 1989; F. M. Ausubel et al., 1992, Current Protocols in Molecular Biology, J. Wiley and Sons, New York, N.Y.

The nucleic acids of the present invention can also be produced by chemical synthesis, e.g., by the phosphoramidite method described by Beaucage et al., 1981, Tetra. Letts. 22:1859-1862, or the triester method according to Matteucci et al., 1981, J. Am. Chem. Soc., 103:3185, and can performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

These nucleic acids can encode full-length variant forms of proteins as well as the wild-type protein. The variant proteins (which could be especially useful for detection and treatment of disorders) will have the variant amino acid sequences encoded by the polymorphisms described in Table 10, when said polymorphisms are read so as to be in-frame with the full-length coding sequence of which it is a component.

Large quantities of the nucleic acids and proteins of the present invention may be prepared by expressing the Gene 216 nucleic acids or portions thereof in vectors or other expression vehicles in compatible prokaryotic or eukaryotic host cells. The most commonly used prokaryotic hosts are strains of Escherichia Coli, although other prokaryotes, such as Bacillus subtilis or Pseudomonas may also be used. Mammalian or other eukaryotic host cells, such as those of yeast, filamentous fungi, plant, insect, or amphibian or avian species, may also be useful for production of the proteins of the present invention. For example, insect cell systems (i.e., lepidopteran host cells and baculovirus expression vectors) are particularly suited for large-scale protein production.

Host cells carrying an expression vector (i.e., transformants or clones) are selected using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

Prokaryotic or eukaryotic cells comprising the nucleic acids of the present invention will be useful not only for the production of the nucleic acids and proteins of the present invention, but also, for example, in studying the characteristics of Gene 216 proteins. Cells and animals that carry the Gene 216 gene can be used as model systems to study and test for substances that have potential as therapeutic agents. The cells are typically cultured mesenchymal stem cells. These may be isolated from individuals with somatic or germline Gene 216 gene. Alternatively, the cell line can be engineered to carry the Gene 216 genes, as described above. After a test substance is applied to the cells, the transformed phenotype of the cell is determined. Any trait of transformed cells can be assessed, including respiratory diseases including asthma, atopy, and response to application of putative therapeutic agents.

Antisense Nucleic Acids

A further embodiment of the invention is antisense nucleic acids or oligonucleotides that are complementary, in whole or in part, to a target molecule comprising a sense strand of Gene 216. The Gene 216 target can be DNA, or its RNA counterpart (i.e., wherein thymine (T) is present in DNA and uracil (U) is present in RNA). When introduced into a cell, antisense nucleic acids or oligonucleotides can hybridize to all or a part of the sense strand of Gene 216, thereby inhibiting gene expression or replication.

In a particular embodiment of the invention, an antisense nucleic acid or oligonucleotide is wholly or partially complementary to, and can hybridize with, a target nucleic acid (either DNA or RNA) having the sequence of SEQ ID NO:1 or SEQ ID NO:6. For example, an antisense nucleic acid or oligonucleotide comprising 16 nucleotides can be sufficient to inhibit expression of the Gene 216 protein. Alternatively, an antisense nucleic acid or oligonucleotide can be complementary to 5′ or 3′ untranslated regions, or can overlap the translation initiation codon (5′ untranslated and translated regions) of the Gene 216 gene, or its functional equivalent. In another embodiment, the antisense nucleic acid is wholly or partially complementary to, and can hybridize with, a target nucleic acid that encodes a Gene 216 polypeptide.

In addition, oligonucleotides can be constructed which will bind to duplex nucleic acid (i.e., DNA:DNA or DNA:RNA), to form a stable triple helix-containing or triplex nucleic acid. Such triplex oligonucleotides can inhibit transcription and/or expression of a gene encoding Gene 216, or its functional equivalent (M.D. Frank-Kamenetskii and S. M. Mirkin, 1995, Ann. Rev. Biochem. 64:65-95). Triplex oligonucleotides are constructed using the base-pairing rules of triple helix formation and the nucleotide sequence of the gene or mRNA for Gene 216.

The present invention encompasses methods of using oligonucleotides in antisense inhibition of the function of Gene 216. In the context of this invention, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art.

In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some non-limiting examples of modifications at the 2′ position of sugar moieties which are useful in the present invention include OH, SH, SCH₃, F, OCH₃, OCN, O(CH₂)ₙ NH₂ and O(CH₂)ₙ CH₃, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with Gene 216 DNA or RNA to inhibit the function thereof.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As defined herein, a “subunit” is a base and sugar combination suitably bound to adjacent subunits through phosphodiester or other bonds.

Antisense nucleic acids or oligonucleotides can be produced by standard techniques (see, e.g., Shewmaker et al., U.S. Pat. No. 5,107,065. The oligonucleotides used in accordance with this invention may be conveniently and routinely made through the well-known technique of solid phase synthesis. Equipment for such synthesis is available from several vendors, including PE Applied Biosystems (Foster City, Calif.). Any other means for such synthesis may also be employed, however, the actual synthesis of the oligonucleotides is well within the abilities of the practitioner. It is also will known to prepare other oligonucleotide such as phosphorothioates and alkylated derivatives.

The oligonucleotides of this invention are designed to be hybridizable with Gene 216 RNA (e.g., mRNA) or DNA. For example, an oligonucleotide (e.g., DNA oligonucleotide) that hybridizes to Gene 216 mRNA can be used to target the mRNA for RnaseH digestion. Alternatively, an oligonucleotide that hybridizes to the translation initiation site of Gene 216 mRNA can be used to prevent translation of the mRNA. In another approach, oligonucleotides that bind to the double-stranded DNA of Gene 216 can be administered. Such oligonucleotides can form a triplex construct and inhibit the transcription of the DNA encoding Gene 216 polypeptides. Triple helix pairing prevents the double helix from opening sufficiently to allow the binding of polymerases, transcription factors, or regulatory molecules. Recent therapeutic advances using triplex DNA have been described (see, e.g., J. E. Gee et al., 1994, Molecular and Immunologic Approaches, Futura Publishing Co., Mt. Kisco, N.Y.).

As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; and 3′ coding region. Preferably, the complementary oligonucleotide is designed to hybridize to the most unique 5′ sequence Gene 216, including any of about 15-35 nucleotides spanning the 5′ coding sequence. Appropriate oligonucleotides can be designed using OLIGO software (Molecular Biology Insights, Inc., Cascade, Co.; http://www.oligo.net).

In accordance with the present invention, the antisense oligonucleotide can be synthesized, formulated as a pharmaceutical composition, and administered to a subject. The synthesis and utilization of antisense and triplex oligonucleotides have been previously described (e.g., H. Simon et al., 1999, Antisense Nucleic Acid Drug Dev. 9:527-31; F. X. Barre et al., 2000, Proc. Natl. Acad. Sci. USA 97:3084-3088; R. Elez et al., 2000, Biochem. Biophys. Res. Commun. 269:352-6; E. R. Sauter et al., 2000, Clin. Cancer Res. 6:654-60). Alternatively, expression vectors derived from retroviruses, adenovirus, herpes or vaccinia viruses, or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding a Gene 216 polypeptide. These techniques are described both in Sambrook et al., 1989 and in Ausubel et al., 1992. For example, Gene 216 expression can be inhibited by transforming a cell or tissue with an expression vector that expresses high levels of untranslatable sense or antisense Gene 216 sequences. Even in the absence of integration into the DNA, such vectors may continue to transcribe RNA molecules until they are disabled by endogenous nucleases. Transient expression may last for a month or more with a non-replicating vector, and even longer if appropriate replication elements included in the vector system.

Various assays may be used to test the ability of Gene 216-specific antisense oligonucleotides to inhibit Gene 216 expression. For example, Gene 216 mRNA levels can be assessed northern blot analysis (Sambrook et al., 1989; Ausubel et al., 1992; J. C. Alwine et al. 1977, Proc. Natl. Acad. Sci. USA 74:5350-5354; I. M. Bird, 1998, Methods Mol. Biol. 105:325-36), quantitative or semi-quantitative RT-PCR analysis (see, e.g., W. M. Freeman et al., 1999, Biotechniques 26:112-122; Ren et al., 1998, Mol. Brain Res. 59:256-63; J. M. Cale et al., 1998, Methods Mol. Biol. 105:351-71), or in situ hybridization (reviewed by A. K. Raap, 1998, Mutat. Res. 400:287-298). Alternatively, antisense oligonucleotides may be assessed by measuring levels of Gene 216 polypeptide, e.g., by western blot analysis, indirect immunofluorescence, immunoprecipitation techniques (see, e.g., J. M. Walker, 1998, Protein Protocols on CD-ROM, Humana Press, Totowa, N.J.). Polypeptides The invention also relates to polypeptides and peptides encoded by the novel nucleic acids described herein. The polypeptides and peptides of this invention can be isolated and/or recombinant. In a preferred embodiment, the Gene 216 polypeptide, or analog or portion thereof, has at least one function characteristic of a Gene 216 protein, for example, proteolysis, adhesion, fusion, antigenic, and intracellular activity. Protein analogs include, for example, naturally-occurring or genetically engineered Gene 216 variants (e.g. mutants) and portions thereof. Variants may differ from wild-type Gene 216 protein by the addition, deletion, or substitution of one or more amino acid residues. In specific embodiments, polypeptide variants are encoded by Gene 216 nucleic acids containing one or more of the SNPs disclosed herein.

Variants also include polypeptides in which one or more residues are modified (i.e., by phosphorylation, sulfation, acylation, etc.), and mutants comprising one or more modified residues. Variant polypeptides can have conservative changes, wherein a substituted amino acid has similar structural or chemical properties, e.g., replacement of leucine with isoleucine. More infrequently, a variant polypeptide can have non-conservative changes, e.g., substitution of a glycine with a tryptophan. Guidance in determining which amino acid residues can be substituted, inserted, or deleted without abolishing biological or immunological activity can be found using computer programs well known in the art, for example, DNASTAR software (DNASTAR, Inc., Madison, Wis.) As non-limiting examples, conservative substitutions in the Gene 216 amino acid sequence can be made in accordance with the following table:

| Original Residue | Conservative Substitution(s) |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunogenicity can be made by selecting substitutions that are less conservative than those shown in the table, above. For example, non-conservative substitutions can be made which more significantly affect the structure of the polypeptide in the area of the alteration, for example, the alpha-helical, or beta-sheet structure; the charge or hydrophobicity of the molecule at the target site; or the bulk of the side chain. The substitutions which generally are expected to produce the greatest changes in the polypeptide's properties are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

In one embodiment, polypeptides of the present invention share at least 50% amino acid sequence identity with a Gene 216 polypeptide, such as SEQ ID NO:4, or fragments thereof. Preferably, the polypeptides share at least 65% amino acid sequence identity; more preferably, the polypeptides share at least 75% amino acid sequence identity; even more preferably, the polypeptides share at least 80% amino acid sequence identity with a Gene 216 polypeptide; still more preferably the polypeptides share at least 90% amino acid sequence identity with a Gene 216 polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch, 1970, J. Mol. Biol. 48:443-453; 2) BLOSSUM62 comparison matrix from Hentikoff and Hentikoff, 1992, Proc. Natl. Acad. Sci. USA 89:10915-10919; 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)* 100. For this calculation, alignment length includes internal gaps but does not include terminal gaps.

In accordance with the present invention, polypeptide sequences may be identical to the sequence of SEQ ID NO:4, or may include up to a certain integer number of amino acid alterations. Polypeptide alterations are selected from the group consisting of at least one amino acid deletion, substitution, including conservative and non-conservative substitution, or insertion. Alterations may occur at the amino- or carboxy-terminal positions of the reference polypeptide sequence or anywhere between those terminal positions, interspersed either individually among the amino acids in the reference sequence or in one or more contiguous groups within the reference sequence. In specific embodiments, polypeptide variants may be encoded by Gene 216 nucleic acids comprising SNPs and/or alternate splice variants.

The invention also relates to isolated, synthesized and/or recombinant portions or fragments of a Gene 216 protein or polypeptide as described herein. Polypeptide fragments (i.e., peptides) can be made which have full or partial function on their own, or which when mixed together (though fully, partially, or nonfunctional alone), spontaneously assemble with one or more other polypeptides to reconstitute a functional protein having at least one functional characteristic of a Gene 216 protein of this invention. In addition, Gene 216 polypeptide fragments may comprise, for example, one or more domains of the Gene 216 polypeptide (e.g., the pre-, pro-, catalytic, cysteine-rich, disintegrin, EGF, transmembrane, and cytoplasmic domains) disclosed herein.

Polypeptides according to the invention can comprise at least 5 amino acid residues; preferably the polypeptides comprise at least 12 residues; more preferably the polypeptides comprise at least 20 residues; and yet more preferably the polypeptides comprise at least 30 residues. Nucleic acids comprising protein-coding sequences can be used to direct the expression of asthma-associated polypeptides in intact cells or in cell-free translation systems. The coding sequence can be tailored, if desired, for more efficient expression in a given host organism, and can be used to synthesize oligonucleotides encoding the desired amino acid sequences. The resulting oligonucleotides can be inserted into an appropriate vector and expressed in a compatible host organism or translation system.

The polypeptides of the present invention, including function-conservative variants, may be isolated from wild-type or mutant cells (e.g., human cells or cell lines), from heterologous organisms or cells (e.g., bacteria, yeast, insect, plant, and mammalian cells), or from cell-free translation systems (e.g., wheat germ, microsomal membrane, or bacterial extracts) in which a protein-coding sequence has been introduced and expressed. Furthermore, the polypeptides may be part of recombinant fusion proteins. The polypeptides can also, advantageously, be made by synthetic chemistry. Polypeptides may be chemically synthesized by commercially available automated procedures, including, without limitation, exclusive solid phase synthesis, partial solid phase methods, fragment condensation or classical solution synthesis.

Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence (e.g., epitope or protein) tag that facilitates purification. Non-limiting examples of epitope tags include c-myc, haemagglutinin (HA), polyhistidine (6×-HIS) (SEQ ID NO:32), GLU-GLU, and DYKDDDDK (SEQ ID NO:33) (FLAG®) epitope tags. Non-limiting examples of protein tags include glutathione-S-transferase (GST), green fluorescent protein (GFP), and maltose binding protein (MBP).

In one approach, the coding sequence of a polypeptide or peptide can be cloned into a vector that creates a fusion with a sequence tag of interest. Suitable vectors include, without limitation, pRSET (Invitrogen Corp., San Diego, Calif.), pGEX (Amersham-Pharmacia Biotech, Inc., Piscataway, N.J.), pEGFP (CLONTECH Laboratories, Inc., Palo Alto, Calif.), and pMAL™ (New England BioLabs (NEB), Inc., Beverly, Mass.) plasmids. Following expression, the epitope, or protein tagged polypeptide or peptide can be purified from a crude lysate of the translation system or host cell by chromatography on an appropriate solid-phase matrix. In some cases, it may be preferable to remove the epitope or protein tag (i.e., via protease cleavage) following purification. As an alternative approach, antibodies produced against a disorder-associated protein or against peptides derived therefrom can be used as purification reagents. Other purification methods are possible.

The present invention also encompasses polypeptide derivatives of Gene 216. The isolated polypeptides may be modified by, for example, phosphorylation, sulfation, acylation, or other protein modifications. They may also be modified with a label capable of providing a detectable signal, either directly or indirectly, including, but not limited to, radioisotopes and fluorescent compounds.

Both the naturally occurring and recombinant forms of the polypeptides of the invention can advantageously be used to screen compounds for binding activity. Many methods of screening for binding activity are known by those skilled in the art and may be used to practice the invention. Several methods of automated assays have been developed in recent years so as to permit screening of tens of thousands of compounds in a short period of time. Such high-throughput screening methods are particularly preferred. The use of high-throughput screening assays to test for inhibitors is greatly facilitated by the availability of large amounts of purified polypeptides, as provided by the invention. The polypeptides of the invention also find use as therapeutic agents as well as antigenic components to prepare antibodies.

The polypeptides of this invention find use as immunogenic components useful as antigens for preparing antibodies by standard methods. It is well known in the art that immunogenic epitopes generally contain at least about five amino acid residues (Ohno et al., 1985, Proc. Natl. Acad. Sci. USA 82:2945). Therefore, the immunogenic components of this invention will typically comprise at least 5 amino acid residues of the sequence of the complete polypeptide chains. Preferably, they will contain at least 7, and most preferably at least about 10 amino acid residues or more to ensure that they will be immunogenic. Whether a given component is immunogenic can readily be determined by routine experimentation Such immunogenic components can be produced by proteolytic cleavage of larger polypeptides or by chemical synthesis or recombinant technology and are thus not limited by proteolytic cleavage sites. The present invention thus encompasses antibodies that specifically recognize asthma-associated immunogenic components.

Structural Studies

A purified Gene 216 polypeptide can be analyzed by well-established methods (e.g., X-ray crystallography, NMR, CD, etc.) to determine the three-dimensional structure of the molecule. The three-dimensional structure, in turn, can be used to model intermolecular interactions. Exemplary methods for crystallization and X-ray crystallography are found in P. G. Jones, 1981, Chemistry in Britain, 17:222-225; C. Jones et al. (eds), Crystallographic Methods and Protocols, Humana Press, Totowa, N.J.; A. McPherson, 198, Preparation and Analysis of Protein Crystals, John Wiley & Sons, New York, N.Y.; T. L. Blundell and L. N. Johnson, 1976, Protein Crystallography, Academic Press, Inc., New York, N.Y.; A. Holden and P. Singer, 1960, Crystals and Crystal Growing, Anchor Books-Doubleday, New York, N.Y.; R. A. Laudise, 1970, The Growth of Single Crystals, Solid State Physical Electronics Series, N. Holonyak, Jr., (ed), Prentice-Hall, Inc.; G. H. Stout and L. H. Jensen, 1989, X-ray Structure Determination: A Practical Guide, 2nd edition, John Wiliey & Sons, New York, N.Y.; Fundamentals of Analytical Chemistry, 3rd. edition, Saunders Golden Sunburst Series, Holt, Rinehart and Winston, Philadelphia, Pa., 1976; P. D. Boyle of the Department of Chemistry of North Carolina State University at http://Iaue.chem.ncsu.edu/web/Grow Xtal.html; M. B. Berry, 1995, Protein Crystalization: Theory and Practice, Structure and Dynamics of E. coli Adenylate Kinase, Doctoral Thesis, Rice University, Houston Tex.; www.bioc.crie.edu/˜berry/papers/crystalization/crystalization.html.

For X-ray diffraction studies, single crystals can be grown to suitable size. Preferably, a crystal has a size of 0.2 to 0.4 mm in at least two of the three dimensions. Crystals can be formed in a solution comprising a Gene 216 polypeptide (e.g., 1.5-200 mg/ml) and reagents that reduce the solubility to conditions close to spontaneous precipitation. Factors that affect the formation of polypeptide crystals include: 1) purity; 2) substrates or co-factors; 3) pH; 4) temperature; 5) polypeptide concentration; and 6) characteristics of the precipitant. Preferably, the Gene 216 polypeptides are pure, i.e., free from contaminating components (at least 95% pure), and free from denatured Gene 216 polypeptides. In particular, polypeptides can be purified by FPLC and HPLC techniques to assure homogeneity (see, Lin et al., 1992, J. Crystal. Growth. 122:242-245). Optionally, Gene 216 polypeptide substrates or co-factors can be added to stabilize the quaternary structure of the protein and promote lattice packing.

Suitable precipitants for crystallization include, but are not limited to, salts (e.g., ammonium sulphate, potassium phosphate); polymers (e.g., polyethylene glycol (PEG) 6000); alcohols (e.g., ethanol); polyalcohols (e.g., 1-methyl-2,4 pentane diol (MPD)); organic solvents; sulfonic dyes; and deionized water. The ability of a salt to precipitate polypeptides can be generally described by the Hofmeister series: PO₄³⁻>HPO₄²⁻=SO₄²⁻>citrate >CH₃CO₂⁻>Cl⁻>Br⁻>NO₃⁻>ClO₄⁻>SCN⁻; and NH₄⁺>K⁺>Na⁺>Li⁺. Non-limiting examples of salt precipitants are shown below (see Berry, 1995).

| Precipitant | Maximum concentration |
|---|---|
| (NH₄⁺/Na⁺/Li⁺)₂ or Mg₂ + SO₄²⁻ | 4.0/1.5/2.1/2.5 M |
| NH₄⁺/Na⁺/K⁺PO₄³⁻ | 3.0/4.0/4.0 M |
| NH₄⁺/K⁺/Na⁺/Li⁺ citrate | ˜1.8 M |
| NH₄⁺/K⁺/Na⁺/Li⁺ acetate | ˜3.0 M |
| NH₄⁺/K⁺/Na⁺/Li⁺ Cl⁻ | 5.2/9.8/4.2/5.4 M |
| NH₄⁺NO₃⁻ | ˜8.0 M |

High molecular weight polymers useful as precipitating agents include polyethylene glycol (PEG), dextran, polyvinyl alcohol, and polyvinyl pyrrolidone (A. Poison et al., 1964, Biochem. Biophys. Acta. 82:463-475). In general, polyethylene glycol (PEG) is the most effective for forming crystals. PEG compounds with molecular weights less than 1000 can be used at concentrations above 40% v/v. PEGs with molecular weights above 1000 can be used at concentration 5-50% w/v. Typically, PEG solutions are mixed with ˜0.1% sodium azide to prevent bacterial growth.

Typically, crystallization requires the addition of buffers and a specific salt content to maintain the proper pH and ionic strength for a protein's stability. Suitable additives include, but are not limited to sodium chloride (e.g., 50-500 mM as additive to PEG and MPD; 0.15-2 M as additive to PEG); potassium chloride (e.g., 0.05-2 M); lithium chloride (e.g., 0.05-2 M); sodium fluoride (e.g., 20-300 mM); ammonium sulfate (e.g., 20-300 mM); lithium sulfate (e.g., 0.05-2 M); sodium or ammonium thiocyanate (e.g., 50-500 mM); MPD (e.g., 0.5-50%); 1,6 hexane diol (e.g., 0.5-10%); 1,2,3 heptane triol (e.g., 0.5-15%); and benzamidine (e.g., 0.5-15%).

Detergents may be used to maintain protein solubility and prevent aggregation. Suitable detergents include, but are not limited to non-ionic detergents such as sugar derivatives, oligoethyleneglycol derivatives, dimethylamine-N-oxides, cholate derivatives, N-octyl hydroxyalkylsulphoxides, sulphobetains, and lipid-like detergents. Sugar-derived detergents include alkyl glucopyranosides (e.g., C8-GP, C9-GP), alkyl thio-glucopyranosides (e.g., C8-tGP), alkyl maltopyranosides (e.g., C10-M, C12-M; CYMAL-3, CYMAL-5, CYMAL-6), alkyl thio-maltopyranosides, alkyl galactopyranosides, alkyl sucroses (e.g., N-octanoylsucrose), and glucamides (e.g., HECAMEG, C-HEGA-10; MEGA-8). Oligoethyleneglycol-derived detergents include alkyl polyoxyethylenes (e.g., C8-E5, C8-En; C12-E8; C12-E9) and phenyl polyoxyethylenes (e.g., Triton X-100). Dimethylamine-N-oxide detergents include, e.g., C10-DAO; DDAO; LDAO. Cholate-derived detergents include, e.g., Deoxy-Big CHAP, digitonin. Lipid-like detergents include phosphocholine compounds. Suitable detergents further include zwitter-ionic detergents (e.g., ZWITTERGENT 3-10; ZWITTERGENT 3-12); and ionic detergents (e.g., SDS).

Crystallization of macromolecules has been performed at temperatures ranging from 60° C. to less than 0° C. However, most molecules can be crystallized at 4° C. or 22° C. Lower temperatures promote stabilization of polypeptides and inhibit bacterial growth. In general, polypeptides are more soluble in salt solutions at lower temperatures (e.g., 4° C.), but less soluble in PEG and MPD solutions at lower temperatures. To allow crystallization at 4° C. or 22° C., the precipitant or protein concentration can be increased or decreased as required. Heating, melting, and cooling of crystals or aggregates can be used to enlarge crystals. In addition, crystallization at both 4° C. and 22° C. can be assessed (A. McPherson, 1992, J. Cryst. Growth. 122:161-167; C. W. Carter, Jr. and C. W. Carter, 1979, J. Biol. Chem. 254:12219-12223; T. Bergfors, 1993, Crystalization Lab Manual).

A crystallization protocol can be adapted to a particular polypeptide or peptide. In particular, the physical and chemical properties of the polypeptide can be considered (e.g., aggregation, stability, adherence to membranes or tubing, internal disulfide linkages, surface cysteines, chelating ions, etc.). For initial experiments, the standard set of crystalization reagents can be used (Hampton Research, Laguna Niguel, Calif.). In addition, the CRYSTOOL program can provide guidance in determining optimal crystallization conditions (Brent Segelke, 1995, Efficiency analysis of sampling protocols used in protein crystallization screening and crystal structure from two novel crystal forms of PLA2, Ph.D. Thesis, University of California, San Diego; http://www. ccp14.ac.uk/ccp/web-mirrors/llnlru pp/crystool/crystool.htm). Exemplary crystallization conditions are shown below (see Berry, 1995).

| | | Concentration | Concentration |
|---|---|---|---|
| Major | | of Major | of |
| Precipitant | Additive | Precipitant | Additive |
| (NH₄)₂SO₄ | PEG 400-2000, MPD, | 2.0-4.0 M | 6%-0.5% |
| | ethanol, or methanol | | |
| Na citrate | PEG 400-2000, MPD, | 1.4-1.8 M | 6%-0.5% |
| | ethanol, or methanol | | |
| PEG 1000- | (NH4)₂SO₄, | 40-50% | 0.2-0.6 M |
| 20000 | NaCl, or Na formate | | |

Robots can be used for automatic screening and optimization of crystallization conditions. For example, the IMPAX and Oryx systems can be used (Douglas Instruments, Ltd., East Garston, United Kingdom). The CRYSTOOL program (Segelke, supra) can be integrated with the robotics programming. In addition, the Xact program can be used to construct, maintain, and record the results of various crystallization experiments (see, e.g., D. E. Brodersen et al., 1999, J. Appl. Cryst. 32: 1012-1016; G. R. Andersen and J. Nyborg, 1996, J. Appl. Cryst. 29:236-240). The Xact program supports multiple users and organizes the results of crystallization experiments into hierarchies. Advantageously, Xact is compatible with both CRYSTOOL and Microsoft® Excel programs.

Four methods are commonly employed to crystallize macromolecules: vapor diffusion, free interface diffusion, batch, and dialysis. The vapor diffusion technique is typically performed by formulating a 1:1 mixture of a solution comprising the polypeptide of interest and a solution containing the precipitant at the final concentration that is to be achieved after vapor equilibration. The drop containing the 1:1 mixture of/protein and precipitant is then suspended and sealed over the well solution, which contains the precipitant at the target concentration, as either a hanging or sitting drop. Vapor diffusion can be used to screen a large number of crystallization conditions or when small amounts of polypeptide are available. For screening, drop sizes of 1 to 2 μl can be used. Once preliminary crystallization conditions have been determined, drop sizes such as 10 μl can be used. Notably, results from hanging drops may be improved with agarose gels (see K. Provost and M. -C. Robert, 1991, J. Cryst. Growth. 110:258-264). Free interface diffusion is performed by layering of a low density solution onto one of higher density, usually in the form of concentrated protein onto concentrated salt. Since the solute to be crystallized must be concentrated, this method typically requires relatively large amounts of protein. However, the method can be adapted to work with small amounts of protein. In a representative experiment, 2 to 5 μl of sample is pipetted into one end of a 20 μl microcapillary pipet. Next, 2 to 5 μl of precipitant is pipetted into the capillary without introducing an air bubble, and the ends of the pipet are sealed. With sufficient amounts of protein, this method can be used to obtain relatively large crystals (see, e.g., S. M. Althoff et al., 1988, J. Mol. Biol. 199:665-666).

The batch technique is performed by mixing concentrated polypeptide with concentrated precipitant to produce a final concentration that is supersaturated for the solute macromolecule. Notably, this method can employ relatively large amounts of solution (e.g., milliliter quantities), and can produce large crystals. For that reason, the batch technique is not recommended for screening initial crystallization conditions.

The dialysis technique is performed by diffusing precipitant molecules through a semipermeable membrane to slowly increase the concentration of the solute inside the membrane. Dialysis tubing can be used to dialyze milliliter quantities of sample, whereas dialysis buttons can be used to dialyze microliter quantities (e.g., 7-200 μl). Dialysis buttons may be constructed out of glass, perspex, or Teflon™ (see, e.g., Cambridge Repetition Engineers Ltd., Greens Road, Cambridge CB43EQ, UK; Hampton Research). Using this method, the precipitating solution can be varied by moving the entire dialysis button or sack into a different solution. In this way, polypeptides can be “reused” until the correct conditions for crystallization are found (see, e.g., C. W. Carter, Jr. et al., 1988, J. Cryst. Growth. 90:60-73). However, this method is not recommended for precipitants comprising concentrated PEG solutions.

Various strategies have been designed to screen crystallization conditions, including 1) pi screening; 2) grid screening; 3) factorials; 4) solubility assays; 5) perturbation; and 6) sparse matrices. In accordance with the pl screening method, the pl of a polypeptide is presumed to be its crystallization point. Screening at the pl can be performed by dialysis against low concentrations of buffer (less than 20 mM) at the appropriate pH, or by use of conventional precipitants.

The grid screening method can be performed on two-dimensional matrices. Typically, the precipitant concentration is plotted against pH. The optimal conditions can be determined for each axis, and then combined. At that point, additional factors can be tested (e.g., temperature, additives). This method works best with fast-forming crystals, and can be readily automated (see M. J. Cox and P. C. Weber, 1988, J. Cryst. Growth. 90:318-324). Grid screens are commercially available for popular precipitants such as ammonium sulphate, PEG 6000, MPD, PEG/LiCl, and NaCl (see, e.g., Hamilton Research).

The incomplete factorial method can be performed by 1) selecting a set of ˜20 conditions; 2) randomly assigning combinations of these conditions; 3) grading the success of the results of each experiment using an objective scale; and 4) statistically evaluating the effects of each of the conditions on crystal formation (see, e.g., C. W. Carter, Jr. et al., 1988, J. Cryst. Growth. 90:60-73). In particular, conditions such as pH, temperature, precipitating agent, and cations can be tested. Dialysis buttons are preferably used with this method. Typically, optimal conditions/combinations can be determined within 35 tests. Similar approaches, such as “footprinting” conditions, may also be employed (see, e.g., E. A. Stura et al., 1991, J. Cryst Growth. 110:1-2).

The perturbation approach can be performed by altering crystallization conditions by introducing a series of additives designed to test the effects of altering the structure of bulk solvent and the solvent dielectric on crystal formation (see, e.g., Whitaker et al., 1995, Biochem. 34:8221-8226). Additives for increasing the solvent dialectric include, but are not limited to, NaCl, KCl, or LiCl (e.g., 200 mM); Na formate (e.g., 200 mM); Na₂HPO₄ or K₂HPO₄ (e.g., 200 mM); urea, triachloroacetate, guanidium HCl, or KSCN (e.g., 20-50 mM). A non-limiting list of additives for decreasing the solvent dialectric include methanol, ethanol, isopropanol, or tert-butanol (e.g., 1-5%); MPD (e.g., 1%); PEG 400, PEG 600, or PEG 1000 (e.g., 1-4%); PEG MME (monomethylether) 550, PEG MME 750, PEG MME 2000 (e.g., 1-4%).

As an alternative to the above-screening methods, the sparse matrix approach can be used (see, e.g., J. Jancarik and S. -H. J. Kim, 1991, Appl. Cryst. 24:409-411; A. McPherson, 1992, J. Cryst. Growth. 122:161-167; B. Cudney et al., 1994, Acta. Cryst. D50:414-423). Sparse matrix screens are commercially available (see, e.g., Hampton Research; Molecular Dimensions, Inc., Apopka, Fla.; Emerald Biostructures, Inc., Lemont, Ill.). Notably, data from Hampton Research sparse matrix screens can be stored and analyzed using ASPRUN software (Douglas Instruments).

Exemplary conditions for an initial screen are shown below (see Berry, 1995).

TABLE 1
Tray 1:

| PEG 8000 (wells 1-6) | | | | | | Ammonium sulfate (wells 7-12) | | | | | |
|---|---|---|---|---|---|---|---|---|---|---|---|
| 1 | 2 | 3 | 4 | 5 | 6 | 7 | 8 | 9 | 10 | 11 | 12 |
| 20% | 20% | 20% | 35% | 35% | 35% | 2.0 M | 2.0 M | 2.0 M | 2.5 M | 2.5 M | 2.5 M |
| pH 5.0 | pH 7.0 | pH 8.6 | pH 5.0 | pH 7.0 | pH 8.6 | pH 5.0 | pH 7.0 | pH 8.8 | pH 5.0 | pH 7.0 | pH 8.8 |
| MPD (wells 13-16) | | | | Na Citrate (wells 17-20) | | | | Na/K Phosphate (wells 21-24) | | | |
| 13 | 14 | 15 | 16 | 17 | 18 | 19 | 20 | 21 | 22 | 23 | 24 |
| 30% | 30% | 50% | 50% | 1.3 M | 1.3 M | 1.5 M | 1.5 M | 2.0 M | 2.0 M | 2.5 M | 2.5 M |
| pH 5.8 | pH 7.6 | pH 5.8 | pH 7.6 | pH 5.8 | pH 7.5 | pH 5.8 | pH 7.5 | pH 6.0 | pH 7.4 | pH 6.0 | pH 7.4 |

Tray 2:

| PEG 2000 MME/0.2 M Ammon. sulfate (wells 25-30) | | | | | |
|---|---|---|---|---|---|
| 25 | 26 | 27 | 28 | 29 | 30 |
| 25% | 25% | 25% | 40% | 40% | 40% |
| pH 5.5 | pH 7.0 | pH 8.5 | pH 5.5 | pH 7.0 | pH 8.5 |

Random for wells 31 to 48

The initial screen can be used with hanging or sitting drops. To conserve the sample, tray 2 can be set up several weeks following tray 1. Wells 31-48 of tray 2 can comprise a random set of solutions. Alternatively, solutions can be formulated using sparse methods. Preferably, test solutions cover a broad range of precipitants, additives, and pH (especially pH 5.0-9.0).

Seeding can be used to trigger nucleation and crystal growth (Stura and Wilson, 1990, J. Cryst. Growth. 110:270-282; C. Thaller et al., 1981, J. Mol. Biol. 147:465-469; A. McPherson and P. Schlichta, 1988, J. Cryst. Growth. 90:47-50). In general, seeding can performed by transferring crystal seeds into a polypeptide solution to allow polypeptide molecules to deposit on the surface of the seeds and produce crystals. Two seeding methods can be used: microseeding and macroseeding. For microseeding, a crystal can be ground into tiny pieces and transferred into the protein solution. Alternatively, seeds can be transferred by adding 1-2 μl of the seed solution directly to the equilibrated protein solution. In another approach, seeds can be transferred by dipping a hair in the seed solution and then streaking the hair across the surface of the drop (streak seeding; see Stura and Wilson, supra). For macroseeding, an intact crystal can be transferred into the protein solution (see, e.g., C. Thaller et al., 1981, J. Mol. Biol. 147:465-469). Preferably, the surface of the crystal seed is washed to regenerate the growing surface prior to being transferred. Optimally, the protein solution for crystallization is close to saturation and the crystal seed is not completely dissolved upon transfer.

Antibodies

An isolated Gene 216 polypeptide or a portion or fragment thereof, can be used as an immunogen to generate anti-Gene 216 antibodies using standard techniques for polyclonal and monoclonal antibody preparation. The full-length Gene 216 polypeptide can be used or, alternatively, the invention provides antigenic peptide fragments of Gene 216 for use as immunogens. The antigenic peptide of Gene 216 comprises at least 5 amino acid residues of the amino acid sequence shown in SEQ ID NO:4, and encompasses an epitope of Gene 216 such that an antibody raised against the peptide forms a specific immune complex with Gene 216 amino acid sequence.

Accordingly, another aspect of the invention pertains to anti-Gene 216 antibodies. The invention provides polyclonal and monoclonal antibodies that bind Gene 216 polypeptides or peptides. The term “monoclonal antibody” or “monoclonal antibody composition”, as used herein, refers to a population of antibody molecules that contain only one species of an antigen binding site capable of immunoreacting with a particular epitope of a Gene 216 polypeptide or peptide. A monoclonal antibody composition thus typically displays a single binding affinity for a particular Gene 216 polypeptide or peptide with which it immunoreacts.

A Gene 216 immunogen typically is used to prepare antibodies by immunizing a suitable subject, (e.g., rabbit, goat, mouse, or other non-human mammal) with the immunogen. An appropriate immunogenic preparation can contain, for example, recombinantly expressed Gene 216 polypeptide or a chemically synthesized Gene 216 polypeptide, or fragments thereof. The preparation can further include an adjuvant, such as Freund's complete or incomplete adjuvant, or similar immunostimulatory agent. Immunization of a suitable subject with an immunogenic Gene 216 preparation induces a polyclonal anti-Gene 216 antibody response.

A number of adjuvants are known and used by those skilled in the art. Non-limiting examples of suitable adjuvants include incomplete Freund's adjuvant, mineral gels such as alum, aluminum phosphate, aluminum hydroxide, aluminum silica, and surface-active substances such as lysolecithin, pluronic polyols, polyanions, peptides, oil emulsions, keyhole limpet hemocyanin, and dinitrophenol. Further examples of adjuvants include N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-Lalanyl-D-isoglutaminyl-L-alanine-2-(1′-2′-dipalmitoyl-sn-glycero-3 hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. A particularly useful adjuvant comprises 5% (wt/vol) squalene, 2.5% Pluronic L121 polymer and 0.2% polysorbate in phosphate buffered saline (Kwak et al., 1992, New Eng. J. Med. 327:1209-1215). Preferred adjuvants include complete BCG, Detox, (RIBI, Immunochem Research Inc.), ISCOMS, and aluminum hydroxide adjuvant (Superphos, Biosector). The effectiveness of an adjuvant may be determined by measuring the amount of antibodies directed against the immunogenic peptide.

Polyclonal anti-Gene 216 antibodies can be prepared as described above by immunizing a suitable subject with a Gene 216 immunogen. The anti-Gene 216 antibody titer in the immunized subject can be monitored over time by standard techniques, such as with an enzyme linked immunosorbent assay (ELISA) using immobilized Gene 216. If desired, the antibody molecules directed against Gene 216 can be isolated from the mammal (e.g., from the blood) and further purified by well-known techniques, such as protein A chromatography to obtain the IgG fraction.

At an appropriate time after immunization, e.g., when the anti-Gene 216 antibody titers are highest, antibody-producing cells can be obtained from the subject and used to prepare monoclonal antibodies by standard techniques, such as the hybridoma technique (see Kohler and Milstein, 1975, Nature 256:495-497; Brown et al., 1981, J. Immunol. 127:539-46; Brown et al., 1980, J. Biol. Chem. 255:4980-83; Yeh et al., 1976, PNAS 76:2927-31; and Yeh et al., 1982, Int. J. Cancer 29:269-75), the human B cell hybridoma technique (Kozbor et al., 1983, Immunol Today 4:72), the EBV-hybridoma technique (Cole et al., 1985, Monoclonal Antibodies and Cancer Therapy, Alan R. Liss, Inc., pp. 77-96) or trioma techniques.

The technology for producing hybridomas is well-known (see generally R. H. Kenneth, 1980, Monoclonal Antibodies: A New Dimension In Biological Analyses, Plenum Publishing Corp., New York, N.Y.; E. A. Lerner, 1981, Yale J. Biol. Med., 54:387-402; M. L. Gefter et al., 1977, Somatic Cell Genet. 3:231-36). In general, an immortal cell line (typically a myeloma) is fused to lymphocytes (typically splenocytes) from a mammal immunized with a Gene 216 immunogen as described above, and the culture supernatants of the resulting hybridoma cells are screened to identify a hybridoma producing a monoclonal antibody that binds Gene 216 polypeptides or peptides.

Any of the many well known protocols used for fusing lymphocytes and immortalized cell lines can be applied for the purpose of generating an anti-Gene 216 monoclonal antibody (see, e.g., G. Galfre et al., 1977, Nature 266:55052; Gefter et al., 1977; Lerner, 1981; Kenneth, 1980). Moreover, the ordinarily skilled worker will appreciate that there are many variations of such methods. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin, and thymidine (HAT medium). Any of a number of myeloma cell lines can be used as a fusion partner according to standard techniques, e.g., the P3-NS1/1-Ag4-1, P3-x63-Ag8.653, or Sp2/O-Ag14 myeloma lines. These myeloma lines are available from ATCC (American Type Culture Collection, Manassas, Va.). Typically, HAT-sensitive mouse myeloma cells are fused to mouse splenocytes using polyethylene glycol (PEG). Hybridoma cells resulting from the fusion arc then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind Gene 216 polypeptides or peptides, e.g., using a standard ELISA assay.

Alternative to preparing monoclonal antibody-secreting hybridomas, a monoclonal anti-Gene 216 antibody can be identified and isolated by screening a recombinant combinatorial immunoglobulin library (e.g., an antibody phage display library) with Gene 216 to thereby isolate immunoglobulin library members that bind Gene 216. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612).

Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al., 1991, Bio/Technology 9:1370-1372; Hay et al., 1992, Hum. Antibod. Hybridomas 3:81-85; Huse et al., 1989, Science 246:1275-1281; Griffiths et al., 1993, EMBO J. 12:725-734; Hawkins et al., 1992, J. Mol. Biol. 226:889-896; Clarkson et al., 1991, Nature 352:624-628; Gram et al., 1992, PNAS 89:3576-3580; Garrad et al., 1991, Bio/Technology 9:1373-1377; Hoogenboom et al., 1991, Nuc. Acid Res. 19:4133-4137; Barbas et al., 1991, PNAS 88:7978-7982; and McCafferty et al., 1990, Nature 348:552-55.

Additionally, recombinant anti-Gene 216 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., 1988, Science 240:1041-1043; Liu et al., 1987, PNAS 84:3439-3443; Liu et al., 1987, J. Immunol. 139:3521-3526; Sun et al., 1987, PNAS 84:214-218; Nishimura et al., 1987, Canc. Res. 47:999-1005; Wood et al., 1985, Nature 314:446-449; and Shaw et al., 1988, J. Natl. Cancer Inst. 80:1553-1559; S. L. Morrison, 1985, Science 229:1202-1207; Oi et al., 1986, BioTechniques 4:214; Winter U.S. Pat. No. 5,225,539; Jones et al., 1986, Nature 321:552-525; Verhoeyan et al., 1988, Science 239:1534; and Bcidler et al., 1988, J. Immunol. 141:4053-4060.

An anti-Gene 216 antibody (e.g., monoclonal antibody) can be used to isolate Gene 216 by standard techniques, such as affinity chromatography or immunoprecipitation. An anti-Gene 216 antibody can also facilitate the purification of natural Gene 216 polypeptide from cells and of recombinantly produced Gene 216 polypeptides or peptides expressed in host cells. Further, an anti-Gene 216 antibody can be used to detect Gene 216 protein (e.g., in a cellular lysate or cell supernatant) in order to evaluate the abundance and pattern of expression of the Gene 216 protein. Anti-Gene 216 antibodies can be used diagnostically to monitor protein levels in tissue as part of a clinical testing procedure, e.g., to, for example, determine the efficacy of a given treatment regimen as described in detail herein. In addition, and anti-Gene 216 antibody can be used as therapeutics for the treatment of diseases related to abnormal Gene 216 expression or function, e.g., asthma.

Ligands

The Gene 216 polypeptides, polynucleotides, variants, or fragments thereof, can be used to screen for ligands (e.g., agonists, antagonists, or inhibitors) that modulate the levels or activity of the Gene 216 polypeptide. In addition, these Gene 216 molecules can be used to identify endogenous ligands that bind to Gene 216 polypeptides or polynucleotides in the cell. In one aspect of the present invention, the full-length Gene 216 polypeptide (e.g., SEQ ID NO:4) is used to identify ligands. Alternatively, variants or fragments of a Gene 216 polypeptide are used. Such fragments may comprise, for example, one or more domains of the Gene 216 polypeptide (e.g., the pre-, pro-, catalytic, cysteine-rich, disintegrin, EGF, transmembrane, and cytoplasmic domains) disclosed herein. Of particular interest are screening assays that identify agents that have relatively low levels of toxicity in human cells. A wide variety of assays may be used for this purpose, including in vitro protein-protein binding assays, electrophoretic mobility shift assays, immunoassays, and the like.

The term “ligand” as used herein describes any molecule, protein, peptide, or compound with the capability of directly or indirectly altering the physiological function, stability, or levels of the Gene 216 polypeptide. Ligands that bind to the Gene 216 polypeptides or polynucleotides of the invention are potentially useful in diagnostic applications and/or pharmaceutical compositions, as described in detail herein. Ligands may encompass numerous chemical classes, though typically they are organic molecules, preferably small organic compounds having a molecular weight of more than 50 and less than about 2,500 daltons. Such ligands can comprise functional groups necessary for structural interaction with proteins, particularly hydrogen bonding, and typically include at least an amine, carbonyl, hydroxyl or carboxyl group, preferably at least two of the functional chemical groups. Ligands often comprise cyclical carbon or heterocyclic structures and/or aromatic or polyaromatic structures substituted with one or more of the above functional groups. Ligands can also comprise biomolecules including peptides, saccharides, fatty acids, steroids, purines, pyrimidines, derivatives, structural analogs, or combinations thereof.

Ligands may include, for example, 1) peptides such as soluble peptides, including Ig-tailed fusion peptides and members of random peptide libraries (see, e.g., Lam et al., 1991, Nature 354:82-84; Houghten et al., 1991, Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al, 1993, Cell 72:767-778); 3) antibodies (e.g., polyclonal, monoclonal, humanized, anti-id iotypic, chimeric, and single chain-antibodies as well as Fab, F(ab′)₂, Fab expression library fragments, and epitope-binding fragments of antibodies); and 4) small organic and inorganic molecules.

Ligands can be obtained from a wide variety of sources including libraries of synthetic or natural compounds. Synthetic compound libraries are commercially available from, for example, Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich Chemical Company, Inc. (Milwaukee, Wis.). Natural compound libraries comprising bacterial, fungal, plant or animal extracts are available from, for example, Pan Laboratories (Bothell, Wash.). In addition, numerous means are available for random and directed synthesis of a wide variety of organic compounds and biomolecules, including expression of randomized oligonucleotides.

Alternatively, libraries of natural compounds in the form of bacterial, fungal, plant and animal extracts can be readily produced. Methods for the synthesis of molecular libraries are readily available (see, e.g., DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carell et al., 1994, Angew. Chem. Int. Ed. Engl. 33:2059; Carell et al., 1994, Angew. Chem. Int. Ed. Engl. 33:2061; and in Gallop et al., 1994, J. Med. Chem. 37:1233). In addition, natural or synthetic compound libraries and compounds can be readily modified through conventional chemical, physical and biochemical means (see, e.g., Blondelle et al., 1996, Trends in Biotech. 14:60), and may be used to produce combinatorial libraries. In another approach, previously identified pharmacological agents can be subjected to directed or random chemical modifications, such as acylation, alkylation, esterification, amidification, and the analogs can be screened for Gene 216-modulating activity.

Numerous methods for producing combinatorial libraries are known in the art, including those involving biological libraries; spatially addressable parallel solid phase or solution phase libraries; synthetic library methods requiring deconvolution the ‘one-bead one-compound’ library method; and synthetic library methods using affinity chromatography selection. The biological library approach is limited to polypeptide libraries, while the other four approaches are applicable to polypeptide, non-peptide oligomer, or small molecule libraries of compounds (K. S. Lam, 1997, Anticancer Drug Des. 12:145).

Libraries may be screened in solution (e.g., Houghten, 1992, Biotechniques 13:412421), or on beads (Lam, 1991, Nature 354:82-84), chips (Fodor, 1993, Nature 364:555-556), bacteria or spores (Ladner U.S. Pat. No. 5,223,409), plasmids (Cull et al., 1992, Proc. Natl. Acad. Sci. USA 89:1865-1869), or on phage (Scott and Smith, 1990, Science 249:386-390; Devlin, 1990, Science 249:404-406; Cwirla et al., 1990, Proc. Natl. Acad. Sci. USA 97:6378-6382; Felici, 1991, J. Mol. Biol. 222:301-310; Ladner, supra).

Where the screening assay is a binding assay, a Gene 216 polypeptide, polynucleotide, analog, or fragment thereof, may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc., that are used to facilitate optimal protein-protein binding and/or reduce non-specific or background interactions. Reagents that improve the efficiency of the assay, such as protease inhibitors, nuclease inhibitors, anti-microbial agents, etc., may be used. The components are added in any order that produces the requisite binding. Incubations are performed at any temperature that facilitates optimal activity, typically between 40 and 40° C. Incubation periods are selected for optimum activity, but may also be optimized to facilitate rapid high-throughput screening. Normally, between 0.1 and 1 hr will be sufficient. In general, a plurality of assay mixtures is run in parallel with different agent concentrations to obtain a differential response to these concentrations. Typically, one of these concentrations serves as a negative control, i.e. at zero concentration or below the level of detection.

To perform cell-free ligand screening assays, it may be desirable to immobilize either the Gene 216 polypeptide, polynucleotide, or fragment to a surface to facilitate identification of ligands that bind to these molecules, as well as to accommodate automation of the assay. For example, a fusion protein comprising a Gene 216 polypeptide and an affinity tag can be produced. In one embodiment, a glutathione-S-transferase/phosphodiesterase fusion protein comprising a Gene 216 polypeptide is adsorbed onto glutathione sepharose beads (Sigma Chemical, St. Louis, Mo.) or glutathione-derivatized microtiter plates. Cell lysates (e.g., containing ³⁵S-labeled polypeptides) are added to the Gene 216-coated beads under conditions to allow complex formation (e.g., at physiological conditions for salt and pH). Following incubation, the Gene 216-coated beads are washed to remove any unbound polypeptides, and the amount of immobilized radiolabel is determined. Alternatively, the complex is dissociated and the radiolabel present in the supernatant is determined. In another approach, the beads are analyzed by SDS-PAGE to identify Gene 216-binding polypeptides.

Ligand-binding assays can be used to identify agonist or antagonists that alter the function or levels of the Gene 216 polypeptide. Such assays are designed to detect the interaction of test agents with Gene 216 polypeptides, polynucleotides, analogs, or fragments thereof. Interactions may be detected by direct measurement of binding. Alternatively, interactions may be detected by indirect indicators of binding, such as stabilization/destabilization of protein structure, or activation/inhibition of biological function. Non-limiting examples of useful ligand-binding assays are detailed below.

Ligands that bind to Gene 216 polypeptides, polynucleotides, analogs, or fragments thereof, can be identified using real-time Bimolecular Interaction Analysis (BIA; Sjolander et al., 1991, Anal. Chem. 63:2338-2345; Szabo et al., 1995, Curr. Opin. Struct. Biol. 5:699-705). BIA-based technology (e.g., BIAcore™; LKB Pharmacia, Sweden) allows study of biospecific interactions in real time, without labeling. In BIA, changes in the optical phenomenon surface plasmon resonance (SPR) is used determine real-time interactions of biological molecules.

Ligands can also be identified by scintillation proximity assays (SPA, described in U.S. Pat. No. 4,568,649). In a modification of this assay that is currently undergoing development, chaperonins are used to distinguish folded and unfolded proteins. A tagged protein is attached to SPA beads, and test agents are added. The bead is then subjected to mild denaturing conditions (such as, e.g., heat, exposure to SDS, etc.) and a purified labeled chaperonin is added. If a test agent binds to a target, the labeled chaperonin will not bind; conversely, if no test agent binds, the protein will undergo some degree of denaturation and the chaperonin will bind.

Ligands can also be identified using a binding assay based on mitochondrial targeting signals (Hurt et al., 1985, EMBO J. 4:2061-2068; Eilers and Schatz, 1986, Nature 322:228-231). In a mitochondrial import assay, expression vectors are constructed in which nucleic acids encoding particular target proteins are inserted downstream of sequences encoding mitochondrial import signals. The chimeric proteins are synthesized and tested for their ability to be imported into isolated mitochondria in the absence and presence of test compounds. A test compound that binds to the target protein should inhibit its uptake into isolated mitochondria in vitro.

The ligand-binding assay described in Fodor et al., 1991, Science 251:767-773, which involves testing the binding affinity of test compounds for a plurality of defined polymers synthesized on a solid substrate, can also be used.

Ligands that bind to Gene 216 polypeptides or peptides can be identified using two-hybrid assays (see, e.g., U.S. Pat. No. 5,283,317; Zervos et al., 1993, Cell 72:223-232; Madura et al., 1993, J. Biol. Chem. 268:12046-12054; Bartel et al., 1993, Biotechniques 14:920-924; Iwabuchi et al., 1993, Oncogene 8:1693-1696; and Brent WO 94/10300). The two-hybrid system relies on the reconstitution of transcription activation activity by association of the DNA-binding and transcription activation domains of a transcriptional activator through protein-protein interaction. The yeast GAL4 transcriptional activator may be used in this way, although other transcription factors have been used and are well known in the art. To carryout the two-hybrid assay, the GAL4 DNA-binding domain, and the GAL4 transcription activation domain are expressed, separately, as fusions to potential interacting polypeptides.

In one embodiment, the “bait” protein comprises a Gene 216 polypeptide fused to the GAL4 DNA-binding domain. The “fish” protein comprises, for example, a human cDNA library encoded polypeptide fused to the GAL4 transcription activation domain. If the two, coexpressed fusion proteins interact in the nucleus of a host cell, a reporter gene (e.g. LacZ) is activated to produce a detectable phenotype. The host cells that show two-hybrid interactions can be used to isolate the containing plasmids containing the cDNA library sequences. These plasmids can be analyzed to determine the nucleic acid sequence and predicted polypeptide sequence of the candidate ligand. Alternatively, methods such as the three-hybrid (Licitra et al., 1996, Proc. Natl. Acad. Sci. USA 93:12817-12821), and reverse two-hybrid (Vidal et al., 1996, Proc. Natl. Acad. Sci. USA 93:10315-10320) systems may be used. Commercially available two-hybrid systems such as the CLONTECH Matchmaker™ systems and protocols (CLONTECH Laboratories, Inc., Palo Alto, Calif.) may be also be used (see also, A. R. Mendelsohn et al., 1994, Curr. Op. Biotech. 5:482; E. M. Phizicky et al., 1995, Microbiological Rev. 59:94; M. Yang et al., 1995, Nucleic Acids Res. 23:1152; S. Fields et al., 1994, Trends Genet. 10:286; and U.S. Pat. No. 6,283,173 and 5,468,614).

Several methods of automated assays have been developed in recent years so as to permit screening of tens of thousands of test agents in a short period of time. High-throughput screening methods are particularly preferred for use with the present invention. The ligand-binding assays described herein can be adapted for high-throughput screens, or alternative screens may be employed. For example, continuous format high throughput screens (CF-HTS) using at least one porous matrix allows the researcher to test large numbers of test agents for a wide range of biological or biochemical activity (see U.S. Pat. No. 5,976,813 to Beutel et al.). Moreover, CF-HTS can be used to perform multi-step assays.

Diagnostics

As discussed herein, chromosomal region 20p13-p12 has been genetically linked to a variety of diseases and disorders, including asthma. The present invention provides nucleic acids and antibodies that can be useful in diagnosing individuals with aberrant Gene 216 expression. In particular, the disclosed SNPs can be used to diagnose chromosomal abnormalities linked to these diseases.

Antibody-Based Diagnostic Methods:

In a further embodiment of the present invention, antibodies which specifically bind to the Gene 216 polypeptide may be used for the diagnosis of conditions or diseases characterized by underexpression or overexpression of the Gene 216 polynucleotide or polypeptide, or in assays to monitor patients being treated with a Gene 216 polypeptide or peptide, or a Gene 216 agonist, antagonist, or inhibitor.

The antibodies useful for diagnostic purposes may be prepared in the same manner as those for use in therapeutic methods, described herein. Antibodies may be raised to the full-length Gene 216 polypeptide sequence (e.g., SEQ ID NO:4). Alternatively, the antibodies may be raised to fragments or variants of the Gene 216 polypeptide. In one aspect of the invention, antibodies are prepared to bind to a Gene 216 polypeptide fragment comprising one or more domains of the Gene 216 polypeptide (e.g., pre-, pro-, catalytic, disintegrin, cysteine-rich, EGF, transmembrane, and cytoplasmic domains) described herein.

Diagnostic assays for the Gene 216 polypeptide include methods that utilize the antibody and a label to detect the protein in biological samples (e.g., human body fluids, cells, tissues, or extracts of cells or tissues). The antibodies may be used with or without modification, and may be labeled by joining them, either covalently or non-covalently, with a reporter molecule. A wide variety of reporter molecules that are known in the art may be used, several of which are described herein.

The invention provides methods for detecting disease-associated antigenic components in a biological sample, which methods comprise the steps of: 1) contacting a sample suspected to contain a disease-associated antigenic component with an antibody specific for an disease-associated antigen, extracellular or intracellular, under conditions in which an antigen-antibody complex can form between the antibody and disease-associated antigenic components in the sample; and 2) detecting any antigen-antibody complex formed in step (1) using any suitable means known in the art, wherein the detection of a complex indicates the presence of disease-associated antigenic components in the sample. It will be understood that assays that utilize antibodies directed against altered Gene 216 amino acid sequences (i.e., epitopes encoded by SNPs, mutations, or variants) are within the scope of the invention.

Many immunoassay formats are known in the art, and the particular format used is determined by the desired application. An immunoassay can use, for example, a monoclonal antibody directed against a single disease-associated epitope, a combination of monoclonal antibodies directed against different epitopes of a single disease-associated antigenic component, monoclonal antibodies directed towards epitopes of different disease-associated antigens, polyclonal antibodies directed towards the same disease-associated antigen, or polyclonal antibodies directed towards different disease-associated antigens. Protocols can also, for example, use solid supports, or may involve immunoprecipitation.

In accordance with the present invention, “competitive” (U.S. Pat. Nos. 3,654,090 and 3,850,752), “sandwich” (U.S. Pat. No. 4,016,043), and “double antibody,” or “DASP” assays may be used. Several procedures for measuring the Gene 216 polypeptide (e.g., ELISA, RIA, and FACS) are known in the art and provide a basis for diagnosing altered or abnormal levels of Gene 216 polypeptide expression. Normal or standard values for Gene 216 polypeptide expression are established by incubating biological samples taken from normal subjects, preferably human, with antibody to the Gene polypeptide under conditions suitable for complex formation. The amount of standard complex formation may be quantified by various methods; photometric means are preferred. Levels of the Gene 216 polypeptide expressed in the subject sample, negative control (normal) sample, and positive control (disease) sample are compared with the standard values. Deviation between standard and subject values establishes the parameters for diagnosing disease.

Typically, immunoassays use either a labeled antibody or a labeled antigenic component (e.g., that competes with the antigen in the sample for binding to the antibody). A number of fluorescent materials are known and can be utilized as labels for antibodies or polypeptides. These include, for example, Cy3, Cy5, Alexa, BODIPY, fluorescein (e.g., FluorX, DTAF, and FITC), rhodamine (e.g., TRITC), auramine, Texas Red, AMCA blue, and Lucifer Yellow. Antibodies or polypeptides can also be labeled with a radioactive element or with an enzyme. Preferred isotopes include ³H, ¹⁴C, 32 P, ³⁵ S, 36 Cl, ⁵¹Cr, ⁵⁷Co, ⁵⁸Co, ⁵⁹ Fe, ⁹⁰Y, ¹²⁵I, ¹³¹I and ¹⁸⁶R Preferred enzymes include peroxidase, β-glucuronidase, β-D-glucosidase, β-D-galactosidase, urease, glucose oxidase plus peroxidase, and alkaline phosphatase (see, e.g., U.S. Pat. Nos. 3,654,090; 3,850,752 and 4,016,043). Enzymes can be conjugated by reaction with bridging molecules such as carbodiimides, diisocyanates, glutaraldehyde, and the like. Enzyme labels can be detected visually, or measured by calorimetric, spectrophotometric, fluorospectrophotometric, amperometric, or gasometric techniques. Other labeling systems, such as avidin/biotin, Tyramide Signal Amplification (TSATM), are known in the art, and are commercially available (see, e.g., ABC kit, Vector Laboratories, Inc., Burlingame, Calif.; NEN®) Life Science Products, Inc., Boston, Mass.).

Kits suitable for antibody-based diagnostic applications typically include one or more of the following components:

(1) Antibodies: The antibodies may be pre-labeled; alternatively, the antibody may be unlabeled and the ingredients for labeling may be included in the kit in separate containers, or a secondary, labeled antibody is provided; and

(2) Reaction components: The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput and/or automated operation.

Nucleic-Acid-Based Diagnostic Methods:

The invention provides methods for altered levels or sequences of Gene 216 nucleic acids in a sample, such as in a biological sample, which methods comprise the steps of: 1) contacting a sample suspected to contain a disease-associated nucleic acid with one or more disease-associated nucleic acid probes under conditions in which hybrids can form between any of the probes and disease-associated nucleic acid in the sample; and 2) detecting any hybrids formed in step (1) using any suitable means known in the art, wherein the detection of hybrids indicates the presence of the disease-associated nucleic acid in the sample. To detect disease-associated nucleic acids present in low levels in biological samples, it may be necessary to amplify the disease-associated sequences or the hybridization signal as part of the diagnostic assay. Techniques for amplification are known to those of skill in the art.

The presence of Gene 216 polynucleotide sequences can be detected by DNA-DNA or DNA-RNA hybridization, or by amplification using probes or primers comprising at least a portion of a Gene 216 polynucleotide, or a sequence complementary thereto. In particular, nucleic acid amplification-based assays can use Gene 216 oligonucleotides or oligomers to detect transformants containing Gene 216 DNA or RNA. Gene 216 nucleic acids useful as probes in diagnostic methods include oligonucleotides at least 15 nucleotides in length, preferably at least 20 nucleotides in length, and most preferably at least 25-55 nucleotides in length, that hybridize specifically with Gene 216 nucleic acids.

Several methods can be used to produce specific probes for Gene 216 polynucleotides. For example, labeled probes can be produced by oligo-labeling, nick translation, end-labeling, or PCR amplification using a labeled nucleotide. Alternatively, Gene 216 polynucleotide sequences (e.g., SEQ ID NO:1 or SEQ ID NO:6), or any portions or fragments thereof, may be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., from Amersham-Pharmacia; Promega Corp.; and U.S. Biochemical Corp., Cleveland, Ohio). Suitable reporter molecules or labels which may be used include radionucleotides, enzymes, fluorescent, chemiluminescent, or chromogenic agents, as well as substrates, cofactors, inhibitors, magnetic particles, and the like.

A sample to be analyzed, such as, for example, a tissue sample (e.g., hair or buccal cavity) or body fluid sample (e.g., blood or saliva), may be contacted directly with the nucleic acid probes. Alternatively, the sample may be treated to extract the nucleic acids contained therein. It will be understood that the particular method used to extract DNA will depend on the nature of the biological sample. The resulting nucleic acid from the sample may be subjected to gel electrophoresis or other size separation techniques, or, the nucleic acid sample may be immobilized on an appropriate solid matrix without size separation.

Kits suitable for nucleic acid-based diagnostic applications typically include the following components:

(1) Probe DNA: The probe DNA may be prelabeled; alternatively, the probe DNA may be unlabeled and the ingredients for labeling may be included in the kit in separate containers; and

(2) Hybridization reagents: The kit may also contain other suitably packaged reagents and materials needed for the particular hybridization protocol, including solid-phase matrices, if applicable, and standards.

In cases where a disease condition is suspected to involve an alteration of the Gene 216 nucleotide sequence, specific oligonucleotides may be constructed and used to assess the level of disease mRNA in cells affected or other tissue affected by the disease. For example, PCR can be used to test whether a person has a disease-related polymorphism (i.e., mutation).

For PCR analysis, Gene 216 oligonucleotides may be chemically synthesized, generated enzymatically, or produced from a recombinant source. Oligomers will preferably comprise two nucleotide sequences, one with a sense orientation (5′→3′) and another with an antisense orientation (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

In accordance with PCR analysis, two oligonucleotides are synthesized by standard methods or are obtained from a commercial supplier of custom-made oligonucleotides. The length and base composition are determined by standard criteria using the Oligo 4.0 primer Picking program (W. Rychlik, 1992; available from Molecular Biology Insights, Inc., Cascade, CO). One of the oligonucleotides is designed so that it will hybridize only to the disease gene DNA under the PCR conditions used. The other oligonucleotide is designed to hybridize a segment of genomic DNA such that amplification of DNA using these oligonucleotide primers produces a conveniently identified DNA fragment. Samples may be obtained from hair follicles, whole blood, or the buccal cavity. The DNA fragment generated by this procedure is sequenced by standard techniques.

In one particular aspect, Gene 216 oligonucleotides can be used to perform Genetic Bit Analysis (GBA) of Gene 216 in accordance with published methods (T. T. Nikiforov et al., 1994, Nucleic Acids Res. 22(20):4167-75; T. T. Nikiforov T T et al., 1994, PCR Methods Appl. 3(5):285-91). In PCR-based GBA, specific fragments of genomic DNA containing the polymorphic site(s) are first amplified by PCR using one unmodified and one phosphorothioate-modified primer. The double-stranded PCR product is rendered single-stranded and then hybridized to immobilized oligonucleotide primer in wells of a multi-well plate. The primer is designed to anneal immediately adjacent to the polymorphic site of interest. The 3′ end of the primer is extended using a mixture of individually labeled dideoxynucleoside triphosphates. The label on the extended base is then determined. Preferably, GBA is performed using semi-automated ELISA or biochip formats (see, e.g., S.R. Head et al., 1997, Nucleic Acids Res. 25(24):5065-71; T. T. Nikiforov et al., 1994, Nucleic Acids Res. 22(20):4167-75).

Other amplification techniques besides PCR may be used as alternatives, such as ligation-mediated PCR or techniques involving Q-beta replicase (Cahill et al., 1991, Clin. Chem., 37(9):1482-5). Products of amplification can be detected by agarose gel electrophoresis, quantitative hybridization, or equivalent techniques for nucleic acid detection known to one skilled in the art of molecular biology (Sambrook et al., 1989). Other alterations in the disease gene may be diagnosed by the same type of amplification-detection procedures, by using oligonucleotides designed to contain and specifically identify those alterations.

Gene 216 polynucleotides may also be used to detect and quantify levels of Gene 216 mRNA in biological samples in which altered expression of Gene 216 polynucleotide may be correlated with disease. These diagnostic assays may be used to distinguish between the absence, presence, increase, and decrease of Gene 216 mRNA levels, and to monitor regulation of Gene 216 polynucleotide levels during therapeutic treatment or intervention. For example, Gene 216 polynucleotide sequences, or fragments, or complementary sequences thereof, can be used in Southern or Northern analysis, dot blot, or other membrane-based technologies; in PCR technologies; or in dip stick, pin, ELISA or biochip assays utilizing fluids or tissues from patient biopsies to detect the status of, e.g., levels or overexpression of Gene 216, or to detect altered Gene 216 expression. Such qualitative or quantitative methods are well known in the art (G. H. Keller and M. M. Manak, 1993, DNA Probes, 2^{nd} Ed, Macmillan Publishers Ltd., England; D. W. Dieffenbach and G. S. Dveksler, 1995, PCR Primer: A Laboratory Manual, Cold Spring Harbor Press, Plainview, N.Y.; B. D. Hames and S. J. Higgins, 1985, Gene Probes 1, 2, IRL Press at Oxford University Press, Oxford, England).

Methods suitable for quantifying the expression of Gene 216 include radiolabeling or biotinylating nucleotides, co-amplification of a control nucleic acid, and standard curves onto which the experimental results are interpolated (P. C. Melby et al., 1993, J. Immunol. Methods 159:235-244; and C. Duplaa et al., 1993, Anal. Biochem. 229-236). The speed of quantifying multiple samples may be accelerated by running the assay in an ELISA format where the oligomer of interest is presented in various dilutions and a spectrophotometric or calorimetric response gives rapid quantification.

In accordance with these methods, the specificity of the probe, i.e., whether it is made from a highly specific region (e.g., at least 8 to 10 or 12 or 15 contiguous nucleotides in the 5′ regulatory region), or a less specific region (e.g., especially in the 3′ coding region), and the stringency of the hybridization or amplification (e.g., high, intermediate, or low) will determine whether the probe identifies only naturally occurring sequences encoding the Gene 216 polypeptide, alleles thereof, or related sequences.

In a particular aspect, a Gene 216 nucleic acid sequence, or a sequence complementary thereto, or fragment thereof, may be useful in assays that detect Gene 216-related diseases such as asthma. The Gene 216 polynucleotide can be labeled by standard methods, and added to a biological sample from a subject under conditions suitable for the formation of hybridization complexes. After a suitable incubation period, the sample can be washed and the signal is quantified and compared with a standard value. If the amount of signal in the test sample is significantly altered from that of a comparable negative control (normal) sample, the altered levels of Gene 216 nucleotide sequence can be correlated with the presence of the associated disease. Such assays may also be used to evaluate the efficacy of a particular prophylactic or therapeutic regimen in animal studies, in clinical trials, or for an individual patient.

To provide a basis for the diagnosis of a disease associated with altered expression of Gene 216, a normal or standard profile for expression is established. This may be accomplished by incubating biological samples taken from normal subjects, either animal or human, with a sequence complementary to the Gene 216 polynucleotide, or a fragment thereof, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for the disease. Deviation between standard and subject (patient) values is used to establish the presence of the condition.

Once the disease is diagnosed and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to diseases such as asthma, the presence of an abnormal amount of Gene 216 transcript in a biological sample (e.g., body fluid, cells, tissues, or cell or tissue extracts) from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the disease.

Microarrays:

In another embodiment of the present invention, oligonucleotides, or longer fragments derived from the Gene 216 polynucleotide sequence described herein may be used as targets in a microarray (e.g., biochip) system. The microarray can be used to monitor the expression level of large numbers of genes simultaneously (to produce a transcript image), and to identify genetic variants, mutations, and polymorphisms. This information may be used to determine gene function, to understand the genetic basis of a disease, to diagnose disease, and to develop and monitor the activities of therapeutic or prophylactic agents. Preparation and use of microarrays have been described in WO 95/11995 to Chee et al.; D. J. Lockhart et al., 1996, Nature Biotechnology 14:1675-1680; M. Schena et al., 1996, Proc. Natl. Acad. Sci. USA 93:10614-10619; U.S. Pat. No. 6,015,702 to P. Lal et al; J. Worley et al., 2000, Microarray Biochip Technology, M. Schena, ed., Biotechniques Book, Natick, MA, pp. 65-86; Y. H. Rogers et al., 1999, Anal. Biochem. 266(1):23-30; S. J. Head et al., 1999, Mol. Cell. Probes. 13(2):81-7; S. J. Watson et al., 2000, Biol. Psychiatry 48(12):1147-56.

In one application of the present invention, microarrays containing arrays of Gene 216 polynucleotide sequences can be used to measure the expression levels of Gene 216 in an individual. In particular, to diagnose an individual with a Gene 216-related condition or disease, a sample from a human or animal (containing nucleic acids, e.g., mRNA) can be used as a probe on a biochip containing an array of Gene 216 polynucleotides (e.g., DNA) in decreasing concentrations (e.g., 1 ng, 0.1 ng, 0.01 ng, etc.). The test sample can be compared to samples from diseased and normal samples. Biochips can also be used to identify Gene 216 mutations or polymorphisms in a population, including but not limited to, deletions, insertions, and mismatches. For example, mutations can be identified by: 1) placing Gene 216 polynucleotides of this invention onto a biochip; 2) taking a test sample (containing, e.g., mRNA) and adding the sample to the biochip; 3) determining if the test samples hybridize to the Gene 216 polynucleotides attached to the chip under various hybridization conditions (see, e.g., V. R. Chechetkin et al., 2000, J. Biomol. Struct. Dyn. 18(1):83-101). Alternatively microarray sequencing can be performed (see, e.g., E. P. Diamandis, 2000, Clin. Chem. 46(10):1523-5).

Chromosome Mapping:

In another application of this invention, the Gene 216 nucleic acid sequence, or a complementary sequence, or fragment thereof, can be used as probes which are useful for mapping the naturally occurring genomic sequence. The sequences may be mapped to a particular chromosome, to a specific region of a chromosome, or to human artificial chromosome constructions (HACs), yeast artificial chromosomes (YACs), bacterial artificial chromosomes (BACs), bacterial Pl constructions, or single chromosome cDNA libraries (see C. M. Price, 1993, Blood Rev., 7:127-134 and by B. J. Trask, 1991, Trends Genet. 7:149-154).

In another of its aspects, the invention relates to a diagnostic kit for detecting Gene 216 polynucleotide or polypeptide as it relates to a disease or susceptibility to a disease, particularly asthma. Also related is a diagnostic kit that can be used to detect or assess asthma conditions. Such kits comprise one or more of the following:

(a) a Gene 216 polynucleotide, preferably the nucleotide sequence of SEQ ID NO:1 or SEQ ID NO:6, or a fragment thereof; or

(b) a nucleotide sequence complementary to that of (a); or

(c) a Gene 216 polypeptide, preferably the polypeptide of SEQ ID NO:4, or a fragment thereof; or

(d) an antibody to a Gene 216 polypeptide, preferably to the polypeptide of SEQ ID NO:4, or an antibody bindable fragment thereof. It will be appreciated that in any such kits, (a), (b), (c), or (d) may comprise a substantial component and that instructions for use can be included. The kits may also contain peripheral reagents such as buffers, stabilizers, etc.

The present invention also includes a test kit for genetic screening that can be utilized to identify mutations in Gene 216. By identifying patients with mutated Gene 216 DNA and comparing the mutation to a database that contains known mutations in Gene 216 and a particular condition or disease, identification and/or confirmation of, a particular condition or disease can be made. Accordingly, such a kit would comprise a PCR-based test that would involve transcribing the patients mRNA with a specific primer, and amplifying the resulting cDNA using another set of primers. The amplified product would be detectable by gel electrophoresis and could be compared with known standards for Gene 216. Preferably, this kit would utilize a patient's blood, serum, or saliva sample, and the DNA would be extracted using standard techniques. Primers flanking a known mutation would then be used to amplify a fragment of Gene 216. The amplified piece would then be sequenced to determine the presence of a mutation.

Genomic Screening:

The use of polymorphic genetic markers linked to the Gene 216 gene is very useful in predicting susceptibility to the diseases genetically linked to 20p13-p12. Similarly, the identification of polymorphic genetic markers within the Gene 216 gene will allow the identification of specific allelic variants that are in linkage disequilibrium with other genetic lesions that affect one of the disease states discussed herein including respiratory disorders, obesity, and inflammatory bowel disease. SSCP (see below) allows the identification of polymorphisms within the genomic and coding region of the disclosed gene. The present invention provides sequences for primers that can be used identify exons that contain SNPs, as well as sequences for primers that can be used to identify the sequence change. This information can be used to identify additional SNPs in accordance with the methods disclosed herein. Suitable methods for genomic screening have also been described by, e.g., Sheffield et al., 1995, Genet., 4:1837-1844; LeBlanc-Straceski et al., 1994, Genomics, 19:341-9; Chen et al., 1995, Genomics, 25:1-8. In employing these methods, the disclosed reagents can be used to predict the risk for disease (e.g., respiratory disorders, obesity, and inflammatory bowel disease) in a population or individual.

Therapeutics

The present invention provides methods of screening for drugs comprising contacting such an agent with a novel protein of this invention or fragment thereof and assaying 1) for the presence of a complex between the agent and the protein or fragment, or 2) for the presence of a complex between the protein or fragment and a ligand, by methods well known in the art. In such competitive binding assays the novel protein or fragment is typically labeled. Free protein or fragment is separated from that present in a protein:protein complex, and the amount of free (i.e., uncomplexed) label is a measure of the binding of the agent being tested to Gene 216 protein or its interference with protein ligand binding, respectively.

This invention also contemplates the use of competitive drug screening assays in which neutralizing antibodies capable of specifically binding the Gene 216 protein compete with a test compound for binding to the Gene 216 protein or fragments thereof. In this manner, the antibodies can be used to detect the presence of any peptide that shares one or more antigenic determinants of a Gene 216 protein.

The goal of rational drug design is to produce structural analogs of biologically active proteins of interest or of small molecules with which they interact (e.g., agonists, antagonists, inhibitors) in order to fashion drugs which are, for example, more active or stable forms of the protein, or which, e.g., enhance or interfere with the function of a protein in vivo (see, e.g., Hodgson, 1991, Bio/Technology, 9:19-21). In one approach, one first determines the three-dimensional structure of a protein of interest or, for example, of the Gene 216 receptor or ligand complex, by x-ray crystallography, by computer modeling or most typically, by a combination of approaches. Less often, useful information regarding the structure of a protein may be gained by modeling based on the structure of homologous proteins. An example of rational drug design is the development of HIV protease inhibitors (Erickson et al., 1990, Science, 249:527-533). In addition, peptides (e.g., Gene 216 protein) are analyzed by an alanine scan (Wells, 1991, Methods in Enzymol., 202:390-411). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original Gene 216 protein. The anti-id could then be used to identify and isolate peptides from banks of chemically or biologically produced banks of peptides. Selected peptides would then act as the pharmacore.

Thus, one may design drugs which result in, for example, altered Gene 216 protein activity or stability or which act as inhibitors, agonists, antagonists, etc. of Gene 216 protein activity. By virtue of the availability of cloned Gene 216 gene sequences, sufficient amounts of the Gene 216 protein may be made available to perform such analytical studies as x-ray crystallography. In addition, the knowledge of the Gene 216 polypeptide sequence will guide those employing computer-modeling techniques in place of, or in addition to x-ray crystallography.

In another aspect of the present invention, cells and animals that carry the Gene 216 gene or an analog thereof can be used as model systems to study and test for substances that have potential as therapeutic agents. After a test substance is administered to animals or applied to the cells, the phenotype of the animals/cells can be determined.

In yet another aspect of this invention, antibodies that specifically react with Gene 216 polypeptide of peptides derived therefrom can be used as therapeutics. In particular, anti-Gene 216 antibodies can be used to block the Gene 216 activity. Anti-Gene 216 antibodies or fragments thereof can be formulated as pharmaceutical compositions and administered to a subject. It is noted that antibody-based therapeutics produced from non-human sources can cause an undesired immune response in human subjects. To minimize this problem, chimeric antibody derivatives can be produced. Chimeric antibodies combine a non-human animal variable region with a human constant region. Chimeric antibodies can be constructed according to methods known in the art (see Morrison et al., 1985, Proc. Natl. Acad. Sci. USA 81:6851; Takeda et al., 1985, Nature 314:452; U.S. Pat. No. 4,816,567 of Cabilly et al.; U.S. Pat. No. 4,816,397 of Boss et al.; European Patent Publication EP 171496; EP 0173494; United Kingdom Patent GB 2177096B). In addition, antibodies can be further “humanized” by any of the techniques known in the art, (e.g., Teng et al., 1983, Proc. Natl. Acad. Sci. USA 80:7308-7312; Kozbor et al., 1983, Immunology Today 4: 7279; Olsson et al., 1982, Meth. Enzymol. 92:3-16; International Patent Application WO92/06193; EP 0239400). Humanized antibodies can also be obtained from commercial sources (e.g., Scotgen Limited, Middlesex, Great Britain). Immunotherapy with a humanized antibody may result in increased long-term effectiveness for the treatment of chronic disease situations or situations requiring repeated antibody treatments.

In one embodiment, compositions (e.g., pharmaceutical compositions) for use with the present invention comprise metalloprotease inhibitors, or analogs or derivatives thereof. Non-limiting examples of metalloprotease inhibitors include: 1) naturally occurring inhibitors, e.g., oprin (J. J. Catanese and L. F. Kress, 1992, Biochemistry 31:410-418; HSF (Y. Yamakawa and T. Omori-Satoh, 1992, J. Biochem. 112:583-589); erinacin (D. Mebs et al., 1996, Toxicon 34:1313-1316; Omori-Satoh et al., 2000, Toxicon 38:1561-1580); DM40 and DM43 (A. G. Neves-Ferreira et al., 2000, Biochem. Biophys. Acta. 1473:309-320); citrate (B. Francis et al., 1992, Toxicon 30:1239-1246); TIMP-1 and TIMP-2 (R. V. Ward et al., 1991, Biochem J. 278, Pt 1:179-873); pyrophosphate (G. S. Makowski and M. L. Ramsby, 1999, Inflammation 23:333-360); proglutamyl peptides such as pyroGlu-Asn-Trp-OH and pyroGlu-Glu-Trp-OH (A. Robeva et al., 1991, Biomed. Biochem. Acta. 50:769-773); 2) peptide analogs and derivatives, e.g., 2-distereomeric furan-2-carbonylamino-3-oxohexahydroindolizino[8,7-b]indole carboxylates (S. D'Alessio et al., 2001, Eur. J. Med. Chem. 36:43-53); phosphonate and carboxylate derivatives of pyroGlu-Asn-Trp-OH (D'Alessio et al., 2001); POL 647 and POL 656 (F. X. Gomis-Ruth et al., 1998, Prot. Sci. 7:283-292); cysteine-switches (K. Nomura and N. Suzuki, 1993, FEBS Lett. 321:84-88); 3) hydroxamate compounds, e.g., batimastat/BB-94 (see, e.g., G. F. Beattie et al., 1998, Clin. Cancer Res. 8:1899-1902); prinomastat/AG3340 (see, e.g., R. Scatena, 2000, Expert Opin. Investig. Drugs 9:2159-2165); and 4) other inhibitors, e.g., ortho-substituted macrocyclic lactams (G. M. Ksander, 1997, J. Med. Chem. 40:495-505); diketopiperazine (DKP) (A. K. Szardenings et al., 1998, J. Med. Chem. 41(13):2194-200; alendronate/PCP (Makowski and Ramsby, 1999); and CT1746 (Z. An et al., 1997, Clin. Exp. Metastasis 15:184-195).

In particular, the determined structures of metalloproteases and metalloprotease inhibitors can be used to devise Gene 216-targeted inhibitors (i.e., by rational drug design; see Szardenings et al., 1998). Structural information can be found in, e.g., C. Oefner et al., 2000, J. Mol. Biol. 296(2):341-9; B. Wu et al., 2000, J. Mol. Biol. 295(2):257-68; L. Chen et al., 1999, J. Mol. Biol. 293(3):545-57; C. Fernandez-Catalanet al., 1998, EMBO J. 17(17):5238-48; S. Arumugam et al., 1998, Biochemistry 37(27):9650-7; Gohlke et al., 1996, FEBS Lett. 378:126-130; Gomis-Ruth et al., 1998; F. X. Gomis-Ruth et al, 1993, EMBO J. 12:4151-4157; F. X. Gomis-Ruth et al, 1996, J. Mol. Biol. 264:556-566; K. Maskos et al., 1998, Proc. Natl. Acad. Sci. USA 95(7):3408-12; F. X. Gomis-Ruth et al, 1997, Nature 389:77-80; M. Betz et al., 1997, Eur. J. Biochem. 247(1):356-63; B. Lovejoy et al., 1994, Biochemistry 33(27):8207-17. Structures of zinc metalloproteases are also found in Molecular Modeling DataBase (MMDB) at the NCBI web site http://www.ncbi.nlm.nih.gov:80/Structure/MMDB/mmdb.shtml (e.g. Accession Nos. 1D5J, 1D8F, 1D7X, 1BSK, 2TLX, 1TLX, 1BUD, 1BSW, 1UEA, 4AIG, 3AIG, 2AIG,1 KUH, 1DTH,1 UMS, IUMT, 7TLN, 6TMN, 5TMN, 5TLN, 4TMN, 4TLN, 3TMN, 2TMN, 1TMN, 1TLP, 1IAG, 1HYT, 1AST, 8TLN, 1THL). In an alternative approach, the binding specificity of TIMP proteins can be engineered to produce inhibitors that specifically inactivate Gene 216 polypeptide (see, e.g., H. Nagase et al., 1999, Ann. NY Acad. Sci. 878:1-11; G. S. Butler et al., 1999, J. Biol. Chem. 274(29):20391-20396).

In another embodiment of the present invention, compositions (e.g., pharmaceutical compositions) for use with the present invention comprise disintegrin agonists, or analogs or derivatives thereof. The determined structures of disintegrin proteins and domains can be used to devise Gene 216 disintegrin-targeted agonists (i.e., by rational drug design). Such structural information can be found in R. A. Atkinson et al., 1994, Int. J. Pept. Protein Res. 43:563-72; V. Saudek et al., 1991, Eur. J. Biochem. 202:329-38; H. Minoux et al., 2000, J. Comput. Aided Mol. Des. 14:317-27.

The present invention contemplates compositions comprising a Gene 216 polynucleotide, polypeptide, antibody, ligand (e.g., agonist, antagonist, or inhibitor), or fragments, variants, or analogs thereof, and a physiologically acceptable carrier, excipient, or diluent as described in detail herein. The present invention further contemplates pharmaceutical compositions useful in practicing the therapeutic methods of this invention. Preferably, a pharmaceutical composition includes, in admixture, a pharmaceutically acceptable excipient (carrier) and one or more of a Gene 216 polypeptide, polynucleotide, ligand, antibody, or fragment or variant thereof, as described herein, as an active ingredient. The preparation of pharmaceutical compositions that contain Gene 216-related reagents as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH-buffering agents, which enhance the effectiveness of the active ingredient.

A Gene 216 polypeptide, polynucleotide, ligand, antibody, or variant or fragment thereof can be formulated into the pharmaceutical composition as neutralized physiologically acceptable salt forms. Suitable salts include the acid addition salts (i.e., formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The pharmaceutical compositions can be administered systemically by oral or parenteral routes. Non-limiting parenteral routes of administration include subcutaneous, intramuscular, intraperitoneal, intravenous, transdermal, inhalation, intranasal, intra-arterial, intrathecal, enteral, sublingual, or rectal. Intravenous administration, for example, can be performed by injection of a unit dose. The term “unit dose” when used in reference to a pharmaceutical composition of the present invention refers to physically discrete units suitable as unitary dosage for humans, each unit containing a predetermined quantity of active material calculated to produce the desired therapeutic effect in association with the required diluent; i.e., carrier, or vehicle.

In one particular embodiment of the present invention, the disclosed pharmaceutical compositions are administered via mucoactive aerosol therapy (see, e.g., M. Fuloria and B. K. Rubin, 2000, Respir. Care 45:868-873; I. Gonda, 2000, J. Pharm. Sci. 89:940-945; R. Dhand, 2000, Curr. Opin. Pulm. Med. 6(1):59-70; B. K. Rubin, 2000, Respir. Care 45(6):684-94; S. Suarez and A. J. Hickey, 2000, Respir. Care. 45(6):652-66).

Pharmaceutical compositions are administered in a manner compatible with the dosage formulation, and in a therapeutically effective amount. The quantity to be administered depends on the subject to be treated, capacity of the subject's immune system to utilize the active ingredient, and degree of modulation of Gene 216 activity desired. Precise amounts of active ingredient required to be administered depend on the judgment of the practitioner and are specific for each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusions sufficient to maintain concentrations of 10 nM to 10 μM in the blood are contemplated. An exemplary pharmaceutical formulation comprises: Gene 216 antagonist or inhibitor (5.0 mg/ml); sodium bisulfite USP (3.2 mg/ml); disodium edetate USP (0.1 mg/ml); and water for injection q.s.a.d. (1.0 ml). As used herein, “pg” means picogram, “ng” means nanogram, “μg” means microgram, “mg” means milligram, “μl” means microliter, “ml” means milliliter, and “l” means L.

For further guidance in preparing pharmaceutical formulations, see, e.g., Gilman et al. (eds), 1990, Goodman and Gilman's: The Pharmacological Basis of Therapeutics, 8th ed., Pergamon Press; and Remington's Pharmaceutical Sciences, 17th ed., 1990, Mack Publishing Co., Easton, Pa.; Avis et al. (eds), 1993, Pharmaceutical Dosage Forms: Parenteral Medications, Dekker, New York; Lieberman et al. (eds), 1990, Pharmaceutical Dosage Forms: Disperse Systems, Dekker, New York.

Pharmacogenetics:

The Gene 216 polypeptides and polynucleotides are also useful in pharmacogenetic analysis (i.e., the study of the relationship between an individual's genotype and that individual's response to a therapeutic composition or drug). See, e.g., M. Eichelbaum, 1996, Clin. Exp. Pharmacol. Physiol. 23(10-11):983-985, and M. W. Linder, 1997, Clin. Chem. 43(2):254-266. The genotype of the individual can determine the way a therapeutic acts on the body or the way the body metabolizes the therapeutic. Further, the activity of drug metabolizing enzymes affects both the intensity and duration of therapeutic activity. Differences in the activity or metabolism of therapeutics can lead to severe toxicity or therapeutic failure. Accordingly, a physician or clinician may consider applying knowledge obtained in relevant pharmacogenetic studies in determining whether to administer a Gene 216 polypeptide, polynucleotide, analog, antagonist, inhibitor, or modulator, as well as tailoring the dosage and/or therapeutic or prophylactic treatment regimen.

In general, two types of pharmacogenetic conditions can be differentiated. Genetic conditions can be due to a single factor that alters the way the drug act on the body (altered drug action), or a factor that alters the way the body metabolizes the drug (altered drug metabolism). These conditions can occur either as rare genetic defects or as naturally-occurring polymorphisms. For example, glucose-6-phosphate dehydrogenase deficiency (G6PD) is a common inherited enzymopathy which results in haemolysis after ingestion of oxidant drugs (anti-malarials, sulfonamides, analgesics, nitrofurans) and consumption of fava beans.

The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. The gene coding for CYP2D6 is highly polymorphic and several mutations have been identified in PM, which all lead to the absence of functional CYP2D6. Poor metabolizers quite frequently experience exaggerated drug response and side effects when they receive standard doses. If a metabolite is the active therapeutic moiety, PM show no therapeutic response. This has been demonstrated for the analgesic effect of codeine mediated by its CYP2D6-formed metabolite morphine. At the other extreme, ultra-rapid metabolizers fail to respond to standard doses. Recent studies have determined that ultra-rapid metabolism is attributable to CYP2D6 gene amplification.

By analogy, genetic polymorphism or mutation may lead to allelic variants of Gene 216 in the population which have different levels of activity. The Gene 216 polypeptides or polynucleotides thereby allow a clinician to ascertain a genetic predisposition that can affect treatment modality. In addition, genetic mutation or variants at other genes may potentiate or diminish the activity of Gene 216-targeted drugs. Thus, in a Gene 216-based treatment, polymorphism or mutation may give rise to individuals that are more or less responsive to treatment. Accordingly, dosage would necessarily be modified to maximize the therapeutic effect within a given population containing the polymorphism. As an alternative to genotyping, specific polymorphic polypeptides or polynucleotides can be identified.

To identify genes that modify Gene 216-targeted drug response, several pharmacogenetic methods can be used. One pharmacogenomics approach, “genome-wide association”, relies primarily on a high-resolution map of the human genome. This high-resolution map shows previously identified gene-related markers (e.g., a “bi-allelic” gene marker map which consists of 60,000-100,000 polymorphic or variable sites on the human genome, each of which has two variants). A high-resolution genetic map can then be compared to a map of the genome of each of a statistically significant number of patients taking part in a Phase II/III drug trial to identify markers associated with a particular observed drug response or side effect. Alternatively, a high-resolution map can be generated from a combination of some ten million known single nucleotide polymorphisms (SNPs) in the human genome. Given a genetic map based on the occurrence of such SNPs, individuals can be grouped into genetic categories depending on a particular pattern of SNPs in their individual genome. In this way, treatment regimens can be tailored to groups of genetically similar individuals, taking into account traits that may be common among such genetically similar individuals (see, e.g., D. R. Pfost et al., 2000, Trends Biotechnol. 18(8):334-8).

As another example, the “candidate gene approach”, can be used. According to this method, if a gene that encodes a drug target is known, all common variants of that gene can be fairly easily identified in the population and it can be determined if having one version of the gene versus another is associated with a particular drug response.

As yet another example, a “gene expression profiling approach”, can be used. This method involves testing the gene expression of an animal treated with a drug (e.g., a Gene 216 polypeptide, polynucleotide, analog, or modulator) to determine whether gene pathways related to toxicity have been turned on.

Information obtained from one of the approaches described herein can be used to establish a pharmacogenetic profile, which can be used to determine appropriate dosage and treatment regimens for prophylactic or therapeutic treatment an individual. A pharmacogenetic profile, when applied to dosing or drug selection, can be used to avoid adverse reactions or therapeutic failure and thus enhance therapeutic or prophylactic efficiency when treating a subject with a Gene 216 polypeptide, polynucleotide, analog, antagonist, inhibitor, or modulator.

Gene 216 polypeptides or polynucleotides are also useful for monitoring therapeutic effects during clinical trials and other treatment. Thus, the therapeutic effectiveness of an agent that is designed to increase or decrease gene expression, polypeptide levels, or activity can be monitored over the course of treatment using the Gene 216 compositions or modulators. For example, monitoring can be performed by: 1) obtaining a pre-administration sample from a subject prior to administration of the agent; 2) detecting the level of expression or activity of the protein in the pre-administration sample; 3) obtaining one or more post-administration samples from the subject; 4) detecting the level of expression or activity of the polypeptide in the post-administration samples; 5) comparing the level of expression or activity of the polypeptide in the pre-administration sample with the polypeptide in the post-administration sample or samples; and 6) increasing or decreasing the administration of the agent to the subject accordingly.

Gene Therapy:

In recent years, significant technological advances have been made in the area of gene therapy for both genetic and acquired diseases (Kay et al., 1997, Proc. Natl. Acad. Sci. USA, 94:12744-12746). Gene therapy can be defined as the transfer of DNA for therapeutic purposes. Improvement in gene transfer methods has allowed for development of gene therapy protocols for the treatment of diverse types of diseases. Gene therapy has also taken advantage of recent advances in the identification of new therapeutic genes, improvement in both viral and non-viral gene delivery systems, better understanding of gene regulation, and improvement in cell isolation and transplantation. Gene therapy would be carried out according to generally accepted methods as described by, for example, Friedman, 1991, Therapy for Genetic Diseases, Friedman, Ed., Oxford University Press, pages 105-121.

Vectors for introduction of genes both for recombination and for extrachromosomal maintenance are known in the art, and any suitable vector may be used. Methods for introducing DNA into cells such as electroporation, calcium phosphate co-precipitation, and viral transduction are known in the art, and the choice of method is within the competence of one skilled in the art (Robbins (ed), 1997, Gene Therapy Protocols, Human Press, NJ). Cells transformed with a Gene 216 gene can be used as model systems to study chromosome 20 disorders and to identify drug treatments for the treatment of such disorders.

Gene transfer systems known in the art may be useful in the practice of the gene therapy methods of the present invention. These include viral and non-viral transfer methods. A number of viruses have been used as gene transfer vectors, including polyoma, i.e., SV40 (Madzak et al., 1992, J. Gen. Virol., 73:1533-1536), adenovirus (Berkner, 1992, Curr. Top. Microbiol. Immunol., 158:39-6; Berkner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; Ohi et al., 1990, Gene, 89:279-282), herpes viruses including HSV and EBV (Margolskee, 1992, Curr. Top. Microbiol. Immunol., 158:67-90; Johnson et al., 1992, J. Virol., 66:2952-2965; Fink et al., 1992, Hum. Gene Ther., 3:11-19; Breakfield et al., 1987, Mol. Neurobiol., 1:337-371; Fresse et al., 1990, Biochem. Pharmacol., 40:2189-2199), and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Most human gene therapy protocols have been based on disabled murine retroviruses.

Non-viral gene transfer methods known in the art include chemical techniques such as calcium phosphate coprecipitation (Graham et al., 1973, Virology, 52:456-467; Pellicer et al., 1980, Science, 209:1414-1422), mechanical techniques, for example microinjection (Anderson et al., 1980, Proc. Natl. Acad. Sci. USA, 77:5399-5403; Gordon et al., 1980, Proc. Natl. Acad. Sci. USA, 77:7380-7384; Brinster et al., 1981, Cell, 27:223-231; Constantini et al., 1981, Nature, 294:92-94), membrane fusion-mediated transfer via liposomes (Felgner et al., 1987, Proc. Natl. Acad. Sci. USA, 84:7413-7417; Wang et al., 1989, Biochemistry, 28:508-9514; Kaneda et al., 1989, J. Biol. Chem., 264:12126-12129; Stewart et al., 1992, Hum. Gene Ther., 3:267-275; Nabel et al., 1990, Science, 249:1285-1288; Lim et al., 1992, Circulation, 83:2007-2011), and direct DNA uptake and receptor-mediated DNA transfer (Wolff et al., 1990, Science, 247:1465-1468; Wu et al., 1991, BioTechniques, 11:474-485; Zenke et al., 1990, Proc. Natl. Acad. Sci. USA, 87:3655-3659; Wu et al., 1989, J. Biol. Chem., 264:16985-16987; Wolff et al., 1991, BioTechniques, 11:474-485; Wagner et al., 1991, Proc. Natl. Acad. Sci. USA, 88:4255-4259; Cotten et al., 1990, Proc. Natl. Acad. Sci. USA, 87:4033-4037; Curiel et al., 1991, Proc. Natl. Acad. Sci. USA, 88:8850-8854; Curiel et al., 1991, Hum. Gene Ther., 3:147-154).

In one approach, plasmid DNA is complexed with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

In another approach, liposome/DNA is used to mediate direct in vivo gene transfer. While in standard liposome preparations the gene transfer process is non-specific, localized in vivo uptake and expression have been reported in tumor deposits, for example, following direct in situ administration (Nabel, 1992, Hum. Gene Ther., 3:399-410).

Suitable gene transfer vectors possess a promoter sequence, preferably a promoter that is cell-specific and placed upstream of the sequence to be expressed. The vectors may also contain, optionally, one or more expressible marker genes for expression as an indication of successful transfection and expression of the nucleic acid sequences contained in the vector. In addition, vectors can be optimized to minimize undesired immunogenicity and maximize long-term expression of the desired gene product(s) (see Nabe, 1999, Proc. Natl. Acad. Sci. USA 96:324-326). Moreover, vectors can be chosen based on cell-type that is targeted for treatment. Notably, gene transfer therapies have been initiated for the treatment of various pulmonary diseases (see, e.g., M. J. Welsh, 1999, J. Clin. Invest. 104(9):1165-6; D. L. Ennist, 1999, Trends Pharmacol. Sci. 20:260-266; S. M. Albelda et al., 2000, Ann. Intern. Med. 132:649-660; E. Alton and C. Kitson C., 2000, Expert Opin. Investig. Drugs. 9(7):1523-35).

Illustrative examples of vehicles or vector constructs for transfection or infection of the host cells include replication-defective viral vectors, DNA virus or RNA virus (retrovirus) vectors, such as adenovirus, herpes simplex virus and adeno-associated viral vectors. Adeno-associated virus vectors are single stranded and allow the efficient delivery of multiple copies of nucleic acid to the cell's nucleus. Preferred are adenovirus vectors. The vectors will normally be substantially free of any prokaryotic DNA and may comprise a number of different functional nucleic acid sequences. An example of such functional sequences may be a DNA region comprising transcriptional and translational initiation and termination regulatory sequences, including promoters (e.g., strong promoters, inducible promoters, and the like) and enhancers which are active in the host cells. Also included as part of the functional sequences is an open reading frame (polynucleotide sequence) encoding a protein of interest. Flanking sequences may also be included for site-directed integration. In some situations, the 5′-flanking sequence will allow homologous recombination, thus changing the nature of the transcriptional initiation region, so as to provide for inducible or non-inducible transcription to increase or decrease the level of transcription, as an example.

In general, the encoded and expressed Gene 216 polypeptide may be intracellular, i.e., retained in the cytoplasm, nucleus, or in an organelle, or may be secreted by the cell. For secretion, the natural signal sequence present in Gene 216 may be retained. When the polypeptide or peptide is a fragment of a Gene 216 protein, a signal sequence may be provided so that, upon secretion and processing at the processing site, the desired protein will have the natural sequence. Specific examples of coding sequences of interest for use in accordance with the present invention include the Gene polypeptide coding sequences, e.g., SEQ ID NO:4.

As previously mentioned, a marker may be present for selection of cells containing the vector construct. The marker may be an inducible or non-inducible gene and will generally allow for positive selection under induction, or without induction, respectively. Examples of marker genes include neomycin, dihydrofolate reductase, glutamine synthetase, and the like. The vector employed will generally also include an origin of replication and other genes that are necessary for replication in the host cells, as routinely employed by those having skill in the art. As an example, the replication system comprising the origin of replication and any proteins associated with replication encoded by a particular virus may be included as part of the construct. The replication system must be selected so that the genes encoding products necessary for replication do not ultimately transform the cells. Such replication systems are represented by replication-defective adenovirus (see G. Acsadi et al., 1994, Hum. Mol. Genet. 3:579-584) and by Epstein-Barr virus. Examples of replication defective vectors, particularly, retroviral vectors that are replication defective, are BAG, (see Price et al., 1987, Proc. Natl. Acad. Sci. USA, 84:156; Sanes et al., 1986, EMBO J., 5:3133). It will be understood that the final gene construct may contain one or more genes of interest, for example, a gene encoding a bioactive metabolic molecule. In addition, cDNA, synthetically produced DNA or chromosomal DNA may be employed utilizing methods and protocols known and practiced by those having skill in the art.

According to one approach for gene therapy, a vector encoding a Gene 216 polypeptide is directly injected into the recipient cells (in vivo gene therapy). Alternatively, cells from the intended recipients are explanted, genetically modified to encode a Gene 216 polypeptide, and reimplanted into the donor (ex vivo gene therapy). An ex vivo approach provides the advantage of efficient viral gene transfer, which is superior to in vivo gene transfer approaches. In accordance with ex vivo gene therapy, the host cells are first transfected with engineered vectors containing at least one gene encoding a Gene 216 polypeptide, suspended in a physiologically acceptable carrier or excipient such as saline or phosphate buffered saline, and the like, and then administered to the host. The desired gene product is expressed by the injected cells, which thus introduce the gene product into the host. The introduced gene products can thereby be utilized to treat or ameliorate a disorder that is related to altered levels of Gene 216 (e.g., asthma).

Animal Models

Gene 216 polynucleotides can be used to generate genetically altered non-human animals or human cell lines. Any non-human animal can be used; however typical animals are rodents, such as mice, rats, or guinea pigs. Genetically engineered animals or cell lines can carry a gene that has been altered to contain deletions, substitutions, insertions, or modifications of the polynucleotide sequence (e.g., exon sequence). Such alterations may render the gene nonfunctional, (i.e., a null mutation) producing a “knockout” animal or cell line. In addition, genetically engineered animals can carry one or more exogenous or non-naturally occurring genes, i.e., “transgenes”, that are derived from different organisms (e.g., humans), or produced by synthetic or recombinant methods. Genetically altered animals or cell lines can be used to study Gene 216 function, regulation, and treatments for Gene 216-related diseases. In particular, knockout animals and cell lines can be used to establish animal models and in vitro models for Gene 216-related illnesses, respectively. In addition, transgenic animals expressing human Gene 216 can be used in drug discovery efforts.

A “transgenic animal” is any animal containing one or more cells bearing genetic information altered or received, directly or indirectly, by deliberate genetic manipulation at a subcellular level, such as by targeted recombination or microinjection or infection with recombinant virus. The term “transgenic animal” is not intended to encompass classical cross-breeding or in vitro fertilization, but rather is meant to encompass animals in which one or more cells are altered by, or receive, a recombinant DNA molecule. This recombinant DNA molecule may be specifically targeted to a defined genetic locus, may be randomly integrated within a chromosome, or it may be extrachromosomally replicating DNA.

Transgenic animals can be selected after treatment of germline cells or zygotes. For example, expression of an exogenous Gene 216 gene or a variant can be achieved by operably linking the gene to a promoter and optionally an enhancer, and then microinjecting the construct into a zygote (see, e.g., Hogan et al., Manipulating the Mouse Embryo, A Laboratory Manual, Cold Spring Harbor Laboratory, Cold Spring Harbor, N.Y.). Such treatments include insertion of the exogenous gene and disrupted homologous genes. Alternatively, the gene(s) of the animals may be disrupted by insertion or deletion mutation of other genetic alterations using conventional techniques (see, e.g., Capecchi, 1989, Science, 244:1288; Valancuis et al., 1991, Mol. Cell Biol., 11:1402; Hasty et al., 1991, Nature, 350:243; Shinkai et al., 1992, Cell, 68:855; Mombaerts et al., 1992, Cell, 68:869; Philpott et al., 1992, Science, 256:1448; Snouwaert et al., 1992, Science, 257:1083; Donehower et al., 1992, Nature, 356:215).

In one aspect of the invention, Gene 216 knockout mice can be produced in accordance with well-known methods (see, e.g., M. R. Capecchi, 1989, Science, 244:1288-1292; P. Li et al., 1995, Cell 80:401-411; L. A. Galli-Taliadoros et al., 1995, J. Immunol. Methods 181(1):1-15; C. H. Westphal et al., 1997, Curr. Biol. 7(7):530-3; S. S. Cheah et al., 2000, Methods Mol. Biol. 136:455-63). The disclosed murine Gene 216 genomic clone can be used to prepare a Gene 216 targeting construct that can disrupt Gene 216 in the mouse by homologous recombination at the Gene 216 chromosomal locus. The targeting construct can comprise a disrupted or deleted Gene 216 sequence that inserts in place of the functioning portion of the native mouse gene. For example, the construct can contain an insertion in the Gene 216 protein-coding region.

Preferably, the targeting construct contains markers for both positive and negative selection. The positive selection marker allows the selective elimination of cells that lack the marker, while the negative selection marker allows the elimination of cells that carry the marker. In particular, the positive selectable marker can be an antibiotic resistance gene, such as the neomycin resistance gene, which can be placed within the coding sequence of Gene 216 to render it non-functional, while at the same time rendering the construct selectable. The herpes simplex virus thymidine kinase (HSV tk) gene is an example of a negative selectable marker that can be used as a second marker to eliminate cells that carry it. Cells with the HSV tk gene are selectively killed in the presence of gangcyclovir. As an example, a positive selection marker can be positioned on a targeting construct within the region of the construct that integrates at the Gene 216 locus. The negative selection marker can be positioned on the targeting construct outside the region that integrates at the Gene 216 locus. Thus, if the entire construct is present in the cell, both positive and negative selection markers will be present. If the construct has integrated into the genome, the positive selection marker will be present, but the negative selection marker will be lost.

The targeting construct can be employed, for example, in embryonal stem cell (ES). ES cells may be obtained from pre-implantation embryos cultured in vitro (M. J. Evans et al., 1981, Nature 292:154-156; M. O. Bradley et al., 1984, Nature 309:255-258; Gossler et al., 1986, Proc. Natl. Acad. Sci. USA 83:9065-9069; Robertson et al., 1986, Nature 322:445-448; S. A. Wood et al., 1993, Proc. Natl. Acad. Sci. USA 90:4582-4584). Targeting constructs can be efficiently introduced into the ES cells by standard techniques such as DNA transfection or by retrovirus-mediated transduction. Following this, the transformed ES cells can be combined with blastocysts from a non-human animal. The introduced ES cells colonize the embryo and contribute to the germ line of the resulting chimeric animal (R. Jaenisch, 1988, Science 240:1468-1474). The use of gene-targeted ES cells in the generation of gene-targeted transgenic mice has been previously described (Thomas et al., 1987, Cell 51:503-512) and is reviewed elsewhere (Frohman et al., 1989, Cell 56:145-147; Capecchi, 1989, Trends in Genet. 5:70-76; Baribault et al., 1989, Mol. Biol. Med. 6:481-492; Wagner, 1990, EMBO J. 9:3025-3032; Bradley et al., 1992, Bio/Technology 10: 534-539).

Several methods can be used to select homologously recombined murine ES cells. One method employs PCR to screen pools of transformant cells for homologous insertion, followed by screening individual clones (Kim et al., 1988, Nucleic Acids Res. 16:8887-8903; Kim et al., 1991, Gene 103:227-233). Another method employs a marker gene is constructed which will only be active if homologous insertion occurs, allowing these recombinants to be selected directly (Sedivy et al., 1989, Proc. Natl. Acad. Sci. USA 86:227-231). For example, the positive-negative selection (PNS) method can be used as described above (see, e.g., Mansour et al., 1988, Nature 336:348-352; Capecchi, 1989, Science 244:1288-1292; Capecchi, 1989, Trends in Genet. 5:70-76). In particular, the PNS method is useful for targeting genes that are expressed at low levels.

The absence of functional Gene 216 in the knockout mice can be confirmed, for example, by RNA analysis, protein expression analysis, and functional studies. For RNA analysis, RNA samples are prepared from different organs of the knockout mice and the Gene 216 transcript is detected in Northern blots using oligonucleotide probes specific for the transcript. For protein expression detection, antibodies that are specific for the Gene 216 polypeptide are used, for example, in flow cytometric analysis, immunohistochemical staining, and activity assays. Alternatively, functional assays are performed using preparations of different cell types collected from the knockout mice.

Several approaches can be used to produce transgenic mice. In one approach, a targeting vector is integrated into ES cell by homologous recombination, an intrachromosomal recombination event is used to eliminate the selectable markers, and only the transgene is left behind (A. L. Joyner et al., 1989, Nature 338(6211):153-6; P. Hasty et al., 1991, Nature 350(6315):243-6; V. Valancius and O. Smithies, 1991, Mol. Cell Biol. 11(3):1402-8; S. Fiering et al., 1993, Proc. Natl. Acad. Sci. USA 90(18):8469-73). In an alternative approach, two or more strains are created; one strain contains the gene knocked-out by homologous recombination, while one or more strains contain transgenes. The knockout strain is crossed with the transgenic strain to produce new line of animals in which the original wild-type allele has been replaced (although not at the same site) with a transgene. Notably, knockout and transgenic animals can be produced by commercial facilities (e.g., The Lerner Research Institute, Cleveland, Ohio; B & K Universal, Inc., Fremont, Calif.; DNX Transgenic Sciences, Cranbury, N.J.; Incyte Genomics, Inc., St. Louis, Mo.).

Transgenic animals (e.g., mice) containing a nucleic acid molecule which encodes human Gene 216, may be used as in vivo models to study the overexpression of Gene 216. Such animals can also be used in drug evaluation and discovery efforts to find compounds effective to inhibit or modulate the activity of Gene 216, such as for example compounds for treating respiratory disorders, diseases, or conditions. One having ordinary skill in the art can use standard techniques to produce transgenic animals which produce human Gene 216 polypeptide, and use the animals in drug evaluation and discovery projects (see, e.g., U.S. Pat. No. 4,873,191 to Wagner; U.S. Pat. No. 4,736,866 to Leder).

In another embodiment of the present invention, the transgenic animal can comprise a recombinant expression vector in which the nucleotide sequence that encodes human Gene 216 is operably linked to a tissue specific promoter whereby the coding sequence is only expressed in that specific tissue. For example, the tissue specific promoter can be a mammary cell specific promoter and the recombinant protein so expressed is recovered from the animal's milk.

In yet another embodiment of the present invention, a Gene 216 “knockout” can be produced by administering to the animal antibodies (e.g., neutralizing antibodies) that specifically recognize an endogenous Gene 216 polypeptide. The antibodies can act to disrupt function of the endogenous Gene 216 polypeptide, and thereby produce a null phenotype. In one specific example, an orthologous mouse Gene 216 polypeptide (e.g., SEQ ID NO:366) or peptide can be used to generate antibodies. These antibodies can be given to a mouse to knockout the function of the mouse Gene 216 ortholog.

In addition, non-mammalian organisms may be used to study Gene 216 and Gene 216-related diseases. For example, model organisms such as C. elegans, D. melanogaster, and S. cerevisiae may be used. Gene 216 homologues can be identified in these model organisms, and mutated or deleted to produce a Gene 216-deficient strain. Human Gene 216 can then be tested for the ability to “complement” the Gene 216-deficient strain. Gene 216-deficient strains can also be used for drug screening. The study of Gene 216 homologs can facilitate the understanding of human Gene 216 biological function, and assist in the identification of binding proteins (e.g., agonists and antagonists).

Gene Identification

To identify genes in the region on 20p13-p12, a set of bacterial artificial chromosome(BAC) clones containing this chromosomal region was identified in accordance with the methods described herein. The BAC clones served as a template for genomic DNA sequencing and served as reagents for identifying coding sequences by direct cDNA selection. Genomic sequencing and direct cDNA selection methods were used to characterize DNA from 20p13-p12.

When one or more genes have been genetically localized to a specific chromosomal region, the gene(s) can be characterized at the molecular level by a series of steps that include: 1) cloning the entire region of DNA in a set of overlapping clones (physical mapping); 2) characterizing the gene(s) encoded by these clones by a combination of direct cDNA selection, exon trapping and DNA sequencing (gene identification); and 3) identifying mutations (i.e., SNPs) in the gene(s) by comparative DNA sequencing of affected and unaffected members of the kindred and/or in unrelated affected individuals and unrelated unaffected controls (mutation analysis).

Physical mapping is accomplished by screening libraries of human DNA cloned in vectors that are propagated in a host such as E. coli, using hybridization or PCR assays from unique molecular landmarks in the chromosomal region of interest. In accordance with the present invention, a physical map of the disorder region was generated by screening a library of human DNA cloned in BACs with a set overgo markers that had been previously mapped to chromosome 20p13-p12 by the efforts of the Human Genome Project. Overgos are unique molecular landmarks in the human genome that can be assayed by hybridization. The location of thousands of overgos on the twenty-two autosomes and two sex chromosomes has been determined through the efforts of the Human Genome Project. For a positional cloning effort, the physical map is tied to the genetic map because the markers used for genetic mapping can also be used as overgos for physical mapping. By screening a BAC library with a combination of overgos derived from genetic markers, genes, and random DNA fragments, a physical map comprised of overlapping clones representing all of the DNA in a chromosomal region of interest can be assembled.

BACs are cloning vectors for large (80 kilobase to 200 kilobase) segments of human or other DNA that are propagated in E. coli. To construct a physical map using BACs, a library of BAC clones is screened so that individual clones harboring the DNA sequence corresponding to a given overgo or set of overgos are identified. Throughout most of the human genome, the overgo markers are spaced approximately 20 to 50 kilobases apart, so that an individual BAC clone typically contains at least two overgo markers. In addition, the BAC libraries that were screened contain enough cloned DNA to cover the human genome twelve times over. An individual overgo typically identifies more than one BAC clone. By screening a twelve-fold coverage BAC library with a series of overgo markers spaced approximately 50 kilobases apart, a physical map consisting of a series of overlapping contiguous BAC clones, i.e., BAC “contigs,” can be assembled for any region of the human genome. This map is closely tied to the genetic map because many of the overgo markers used to prepare the physical map are also genetic markers.

When constructing a physical map, it often happens that there are gaps in the overgo map of the genome that result in the inability to identify BAC clones that are overlapping in a given location. Typically, the physical map is first constructed from a set of overgos identified through the publicly available literature and World Wide Web resources. The initial map consists of several separate BAC contigs that are separated by gaps of unknown molecular distance. To identify BAC clones that fill these gaps, it is necessary to develop new overgo markers from the ends of the clones on either side of the gap. This is done by sequencing the terminal 200 to 300 base pairs of the BACs flanking the gap, and developing a PCR or hybridization based assay. If the terminal sequences are demonstrated to be unique within the human genome, then the new overgo can be used to screen the BAC library to identify additional BACs that contain the DNA from the gap in the physical map. To assemble a BAC contig that covers a region the size of the disorder region (6,000,000 or more base pairs), it is necessary to develop new overgo markers from the ends of a number of clones.

After building a BAC contig, this set of overlapping clones serves as a template for identifying the genes encoded in the chromosomal region. Gene identification can be accomplished by many methods. Three methods are commonly used: 1) a set of BACs selected from the BAC contig to represent the entire chromosomal region are sequenced, and computational methods are used to identify all of the genes; 2) the BACs from the BAC contig are used as a reagent to clone cDNAs corresponding to the genes encoded in the region by a method termed direct cDNA selection; or 3) the BACs from the BAC contig are used to identify coding sequences by selecting for specific DNA sequence motifs in a procedure called exon trapping. Gene 216 was identified by methods (1) and (2) in accordance with the techniques disclosed herein.

To sequence the entire BAC contig representing the disorder region, a set of BACs can be chosen for subcloning into plasmid vectors and subsequent DNA sequencing of these subclones. Since the DNA cloned in the BACs represents genomic DNA, this sequencing is referred to as genomic sequencing to distinguish it from cDNA sequencing. To initiate the genomic sequencing for a chromosomal region of interest, several non-overlapping BAC clones are chosen. DNA for each BAC clone is prepared, and the clones are sheared into random small fragments that are subsequently cloned into standard plasmid vectors such as pUC18. The plasmid clones are then grown to propagate the smaller fragments, and these are the templates for sequencing. To ensure adequate coverage and sequence quality for the BAC DNA sequence, sufficient plasmid clones are sequenced to yield three-fold coverage of the BAC clone. For example, if the BAC is 100 kilobases long, then phagemids are sequenced to yield 300 kilobases of sequence. Since the BAC DNA is randomly sheared prior to cloning in the phagemid vector, the 300 kilobases of raw DNA sequence can be assembled by computational methods into overlapping DNA sequences termed sequence contigs. For the purposes of initial gene identification by computational methods, three-fold coverage of each BAC is sufficient to yield twenty to forty sequence contigs of 1000 base pairs to 20,000 base pairs.

In accordance with the present invention, the “seed” BACs from the BAC contig in the disorder region were sequenced. The sequence of the “seed” BACs was then used to identify minimally overlapping BACs from the contig, and these were subsequently sequenced. In this manner, the entire candidate region can be sequenced, with several small sequence gaps left in each BAC. This sequence serves as the template for computational gene identification. In one approach, genes can be identified by comparing the sequence of BAC contig to publicly available databases of cDNA and genomic sequences, e.g. UniGene, dbEST, EMBL nucleotide database, GenBank, and the DNA Database of Japan (DDBJ). The BAC DNA sequence can also be translated into protein sequence, and the protein sequence can be used to search publicly available protein databases, e.g., GenPept, EMBL protein database, Protein Information Resource (PIR), Protein Data Bank (PDB), and SWISS-PROT. These comparisons are typically done using the BLAST family of computer algorithms and programs (Altschul et al., 1990, J. Mol. Biol., 215:403-410; Altschul et al, 1997, Nucl. Acids Res., 25:3389-3402).

For nucleotide queries, BLASTN, BLASTX, and TBLASTX can be used. BLASTN compares a nucleotide query sequence with a nucleotide sequence database; BLASTX compares a nucleotide query sequence translated in all reading frames against a protein sequence database; TBLASTX compares the six-frame translations of a nucleotide query sequence against the six-frame translations of a nucleotide sequence database. For protein queries, BLASTP and TBLASTN can be used. BLASTP compares a protein query sequence with a protein sequence database; TBLASTN compares a protein query sequence against a nucleotide sequence database dynamically translated in all reading frames.

Additionally, computer algorithms such as MZEF (Zhang, 1997, Proc. Natl. Acad. Sci. USA 94:565-568), GRAIL (Uberbacher et al., 1996, Methods Enzymol., 266:259-281), and Genscan (Burge and Karlin, 1997, J. Mol. Biol., 268:78-94) can be used to predict the location of exons in the sequence based on the presence of specific DNA sequence motifs that are common to all exons, as well as the presence of codon usage typical of human protein encoding sequences.

In addition to identifying genes by computational methods, genes can be identified by direct cDNA selection (Del Mastro and Lovett, 1996, Methods in Molecular Biology, Humana Press Inc., NJ). In direct cDNA selection, cDNA pools from tissues of interest are prepared, and BACs from the candidate region are used in a liquid hybridization assay to capture the cDNAs which base pair to coding regions in the BAC. In the methods described herein, the cDNA pools were created from several different tissues by random priming and oligo dT priming the first strand cDNA from poly A⁺ RNA, synthesizing the second-strand cDNA by standard methods, and adding linkers to the ends of the cDNA fragments. In this approach, the linkers are used to amplify the cDNA pools of BAC clones from the disorder region identified by screening a BAC library. The amplified products are then used as a template for initiating DNA synthesis to create a biotin labeled copy of BAC DNA. Following this, the biotin labeled copy of the BAC DNA is denatured and incubated with an excess of the PCR amplified, linkered cDNA pools which have also been denatured. The BAC DNA and cDNA are allowed to anneal in solution, and heteroduplexes between the BAC and the cDNA are isolated using streptavidin coated magnetic beads. The cDNAs that are captured by the BAC are then amplified using primers complimentary to the linker sequences, and the hybridization/selection process is repeated for a second round. After two rounds of direct cDNA selection, the cDNA fragments are cloned, and a library of these direct selected fragments is created.

The cDNA clones isolated by direct selection are analyzed by two methods. Where the genomic target DNA sequence is obtained from a pool of BACs from the disorder region, the cDNAs are mapped to BAC genomic clones to verify their chromosomal location. This is accomplished by arraying the cDNAs in microtiter dishes, and replicating their DNA in high-density grids. Individual genomic clones known to map to the region are then hybridized to the grid to identify direct selected cDNAs mapping to that region. cDNA clones that are confirmed to correspond to individual BACs are sequenced. To determine whether the cDNA clones isolated by direct selection share sequence identity or similarity to previously identified genes, the DNA and protein coding sequences are compared to publicly available databases using the BLAST family of programs described above.

The combination of genomic DNA sequence and cDNA sequence provided by BAC sequencing and by direct cDNA selection yields an initial list of putative genes in the region. In the present invention, the genes in the region were candidates for the asthma locus. To further characterize each gene, Northern blots were performed to determine the size of the transcript corresponding to each gene, and to determine which putative exons were transcribed together to make an individual gene. For Northern blot analysis of each gene, probes are prepared from direct selected cDNA clones or by PCR amplifying specific fragments from genomic DNA, cDNA or from the BAC encoding the putative gene of interest. The Northern blot analysis is used to determine the size of the transcript and the tissues in which it is expressed. For transcripts that are not highly expressed, it is sometimes necessary to perform a reverse transcription PCR assay using RNA from the tissues of interest as a template for the reaction.

Gene identification by computational methods and by direct cDNA selection provides unique information about the genes in a region of a chromosome. Once genes are identified, it is possible to examine subjects for sequence variants. Variant sequences can be inherited as allelic differences or can arise from spontaneous mutations.

Inherited alleles can be analyzed for linkage to a disease susceptibility locus. Linkage analysis is possible because of the nature of inheritance of chromosomes from parents to offspring. During meiosis, the two parental homologs pair to guide their proper separation to daughter cells. While they are paired, the two homologs exchange pieces of the chromosomes, in an event called “crossing over” or “recombination.” The resulting chromosomes contain parts that originate from both parental homologs. The closer together two sequences are on the chromosome, the less likely that a recombination event will occur between them, and the more closely linked they are.

In the present invention, data obtained from the different families were combined and analyzed together by a computer using statistical methods described herein. The results were then used as evidence for linkage between the genetic markers used and an asthma susceptibility locus.

In general, a recombination frequency of 1% is equivalent to approximately 1 map unit, a relationship that holds up to frequencies of about 20% or 20 cM. One centimorgan (cM) is roughly equivalent to 1,000 kb of DNA. The entire human genome is 3,300 cM long. In order to find an unknown disease gene within 5-10 cM of a marker locus, the whole human genome can be searched with roughly 330 informative marker loci spaced at approximately 10 cM intervals (Botstein et al., 1980, Am. J. Hum. Genet., 32:314-331).

The reliability of linkage results is established by using a number of statistical methods. The methods most commonly used for the detection by linkage analysis of oligogenes involved in the etiology of a complex trait are non-parametric or model-free methods which have been implemented into the computer programs MAPMAKER/SIBS (L. Kruglyak and E. S. Lander, 1995, Am. J. Hum. Genet. 57:439-454) and GENEHUNTER (L. Kruglyak et al., 1996, Am. J. Hum. Genet. 58:1347-1363). Typically, linkage analysis is performed by typing members of families with multiple affected individuals at a given marker locus and evaluating if the affected members (excluding parent-offspring pairs) share alleles at the marker locus that are identical by descent (IBD) more often than expected by chance alone.

As a result of the rapid advances in mapping the human genome over the last few years, and concomitant improvements in computer methodology, it has become feasible to carry out linkage analyses using multi-point data. Multi-point analysis provides a simultaneous analysis of linkage between the trait and several linked genetic markers, when the recombination distance among the markers is known. A LOD score statistic is computed at multiple locations along a chromosome to measure the evidence that a susceptibility locus is located nearby. A LOD score is the logarithm base 10 of the ratio of the likelihood that a susceptibility locus exists at a given location to the likelihood that no susceptibility locus is located there. By convention, when testing a single marker, a total LOD score greater than +3.0 (that is, odds of linkage being 1,000 times greater than odds of no linkage) is considered to be significant evidence for linkage.

Multi-point analysis is advantageous for two reasons. First, the informativeness of the pedigrees is usually increased. Each pedigree has a certain amount of potential information, dependent on the number of parents heterozygous for the marker loci and the number of affected individuals in the family. However, few markers are sufficiently polymorphic as to be informative in all those individuals. If multiple markers are considered simultaneously, then the probability of an individual being heterozygous for at least one of the markers is greatly increased. Second, an indication of the position of the disease gene among the markers may be determined. This allows identification of flanking markers, and thus eventually allows identification of a small region in which the disease gene resides.

### EXAMPLES

The examples as set forth herein are meant to exemplify the various aspects of the present invention and are not intended to limit the invention in any way.

### EXAMPLE 1

### Family Collection

Asthma is a complex disorder that is influenced by a variety of factors, including both genetic and environmental effects. Complex disorders are typically caused by multiple interacting genes, some contributing to disease development and some conferring a protective effect. The success of linkage analyses in identifying chromosomes with significant LOD scores is achieved in part as a result of an experimental design tailored to the detection of susceptibility genes in complex diseases, even in the presence of epistasis and genetic heterogeneity. Also important are rigorous efforts in ascertaining asthmatic families that meet strict guidelines, and collecting accurate clinical information.

Given the complex nature of the asthma phenotype, non-parametric affected sib pair analyses were used to analyze the genetic data. This approach does not require parameter specifications such as mode of inheritance, disease allele frequency, penetrance of the disorder, or phenocopy rates. Instead, it determines whether the inheritance pattern of a chromosomal region is consistent with random segregation. If it is not, affected sibs inherit identical copies of alleles more often than expected by chance. Because no models for inheritance are assumed, allele-sharing methods tend to be more robust than parametric methods when analyzing complex disorders. They do, however, require larger sample sizes to reach statistically significant results.

At the outset of the program, the goal was to collect 400 affected sib-pair families for the linkage analyses. Based on a genome scan with markers spaced ˜10 cM apart, this number of families was predicted to provide >95% power to detect an asthma susceptibility gene that caused an increased risk to first-degree relatives of 3-fold or greater. The assumed relative risk of 3-fold was consistent with epidemiological studies in the literature that suggest an increased risk ranging from 3- to 7-fold. The relative risk was based on gender, different classifications of the asthma phenotype (i.e. bronchial hyper-responsiveness versus physician's diagnosis) and, in the case of offspring, whether one or both parents were asthmatic.

The family collection efforts exceeded the initial goal of 400, obtaining a total of 444 affected sibling pair (ASP) families, with 342 families from the UK and 102 families from the US. The ASP families in the US collection were Caucasian with a minimum of two affected siblings that were identified through both private practice and community physicians as well as through advertising. A total of 102 families were collected in Kansas, Nebraska, and Southern California. In the UK collection, Caucasian families with a minimum of two affected siblings were identified through physicians' registers in a region surrounding Southampton and including the Isle of Wight. In both the US and UK collections, additional affected and unaffected sibs were collected whenever possible. An additional 39 families from the United Kingdom were utilized from an earlier collection effort with different ascertainment criteria. These families were recruited either: 1) without reference to asthma and atopy; or 2) by having at least one family member or at least two family members affected with asthma. The randomly ascertained samples were identified from general practitioner registers in the Southampton area. For families with affected members, the probands were recruited from hospital based clinics in Southampton. Seven pedigrees extended beyond a single nuclear family.

Families were included in the study if they met all of the following criteria: 1) the biological mother and biological father were Caucasian and agreed to participate in the study; 2) at least two biological siblings were alive, each with a current physician diagnosis of asthma, and were 5 to 21 years of age; and 3) the two siblings were currently taking asthma medications on a regular basis. This included regular, intermittent use of inhaled or oral bronchodilators and regular use of cromolyn, theophylline, or steroids.

Families were excluded from the study if they met any one of the following criteria: 1) both parents were affected (i.e., with a current diagnosis of asthma, having asthma symptoms, or on asthma medications at the time of the study); 2) any of the siblings to be included in the study was less than 5 years of age; 3) any asthmatic family member to be included in the study was taking beta-blockers at the time of the study, 4) any family member to be included in the study had congenital or acquired pulmonary disease at birth (e.g. cystic fibrosis), a history of serious cardiac disease (myocardial infarction) or any history of serious pulmonary disease (e.g. emphysema); or 5) any family member to be included in the study was pregnant.

An extensive clinical instrument was designed and data from all participating family members were collected. The case report form (CRF) included questions on demographics, medical history including medications, a health survey on the incidence and frequency of asthma, wheeze, eczema, hay fever, nasal problems, smoking, and questions on home environment. Data from a video questionnaire designed to show various examples of wheeze and asthmatic attacks were also included in the CRF. Clinical data, including skin prick tests to 8 common allergens, total and specific IgE levels, and bronchial hyper-responsiveness following a methacholine challenge, were also collected from all participating family members. All data were entered into a SAS dataset by IMTCI, a CRO; either by double data entry or scanning followed by on-screen visual validation. An extensive automated review of the data was performed on a routine basis and a full audit at the conclusion of the data entry was completed to verify the accuracy of the dataset.

### EXAMPLE 2

### Genome Scan

In order to identify chromosomal regions linked to asthma, the inheritance pattern of alleles from genetic markers spanning the genome was assessed on the collected family resources. As described above, combining these results with the segregation of the asthma phenotype in these families allows the identification of genetic markers that are tightly linked to asthma. In turn, this provides an indication of the location of genes predisposing affected individuals to asthma. The genotyping strategy was twofold: 1) to conduct a genome wide scan using markers spaced at approximately 10 cM intervals; and 2) to target ten chromosomal regions for high density genetic mapping. The initial candidate regions for high-density mapping were chosen based on suggestions of linkage to these regions by other investigators.

Genotypes of PCR amplified simple sequence microsatellite genetic linkage markers were determined using ABI model 377 Automated Sequencers (PE Applied Biosystems). Microsatellite markers were obtained from Research Genetics Inc. (Huntsville, Ala.) in the fluorescent dye-conjugated form (see Dubovsky et al., 1995, Hum. Mol. Genet. 4(3):449-452). The markers comprised a variation of a human linkage mapping panel as released from the Cooperative Human Linkage Center (CHLC), also known as the Weber lab screening set version 8. The variation of the Weber 8 screening set consisted of 529 markers with an average spacing of 6.9 cM (autosomes only) and 7.0 cM (all chromosomes). Eighty-nine percent of the markers consisted of either tri- or tetra-nucleotide microsatellites. There were no gaps present in chromosomal coverage greater than 17.5 cM.

Study subject genomic DNA (5 μl; 4.5 ng/μl) was amplified in a 10 μl PCR reaction using AmpliTaq Gold DNA polymerase (0.225 U); 1× PCR buffer (80 mM (NH₄)₂SO₄; 30 mM Tris-HCl (pH 8.8); 0.5% Tween-20); 200 μM each dATP, dCTP, dGTP and dTTP; 1.5-3.5 μM MgCl₂; and 250 μM forward and reverse PCR primers. PCR reactions were set up in 192 well plates (Costar) using a Tecan Genesis 150 robotic workstation equipped with a refrigerated deck. PCR reactions were overlaid with 20 μl mineral oil, and thermocycled on an MJ Research Tetrad DNA Engine equipped with four 192 well heads using the following conditions: 92° C. for 3 min; 6 cycles of 92° C. for 30 sec, 56° C. for 1 min, 72° C. for 45 sec; followed by 20 cycles of 92° C. for 30 sec, 55° C. for 1 min, 72° C. for 45 sec; and a 6 min incubation at 72° C.

PCR products of 8-12 microsatellite markers were subsequently pooled into two 96-well microtitre plates (2.0 μl PCR product from TET and FAM labeled markers, 3.0 μl HEX labeled markers) using a Tecan Genesis 200 robotic workstation and brought to a final volume of 25 μl with H₂O. Following this, 1.9 μl of pooled PCR product was transferred to a loading plate and combined with 3.0 μl loading buffer (2.5 μl formamide/blue dextran (9.0 mg/ml), 0.5 μl GS-500 TAMRA labeled size standard, ABI). Samples were denatured in the loading plate for 4 min at 95° C., placed on ice for 2 min, and electrophoresed on a 5% denaturing polyacrylamide gel (FMC on the ABI 377XL). Samples (0.8 μl) were loaded onto the gel using an 8 channel Hamilton Syringe pipettor.

Each gel consisted of 62 study subjects and 2 control subjects (CEPH parents ID #1331-01 and 1331-02, Coriell Cell Repository, Camden, N.J.). Genotyping gels were scored in duplicate by investigators blind to patient identity and affection status using GENOTYPER analysis software V 1.1.12 (ABI; PE Applied Biosystems). Nuclear families were loaded onto the gel with the parents flanking the siblings to facilitate error detection. The final tables obtained from the GENOTYPER output for each gel analysed were imported into a SYBASE Database.

Allele calling (binning) was performed using the SYBASE version of the ABAS software (Ghosh et al., 1997, Genome Research 7:165-178). Offsize bins were checked manually and incorrect calls were corrected or blanked. The binned alleles were then imported into the program MENDEL (Lange et al., 1988, Genetic Epidemiology, 5:471) for inheritance checking using the USERM13 subroutine (Boehnke et al., 1991, Am. J. Hum. Genet. 48:22-25). Non-inheritance was investigated by examining the genotyping traces and, once all discrepancies were resolved, the subroutine USERM13 was used to estimate allele frequencies.

### EXAMPLE 3

### Linkage Analysis

Chromosomal regions harboring asthma susceptibility genes by linkage analysis of genotyping data and three separate phenotypes, asthma, bronchial hyper-responsiveness, and atopic status were identified as follows.

1. Asthma Phenotype:

For the initial linkage analysis, the phenotype and asthma affection status were defined by a patient who answered the following questions in the affirmative: i) have you ever had asthma; ii) do you have a current physician's diagnosis of asthma; and iii) are you currently taking asthma medications? Medications included inhaled or oral bronchodilators, cromolyn, theophylline, or steroids. Multipoint linkage analyses of allele sharing in affected individuals were performed using the MAPMAKER/SIBS analysis program (L. Kruglyak and E. S. Lander, 1995, Am. J. Hum. Genet. 57:439-454). The map location and distances between markers were obtained from the genetic maps published by the Marshfield medical research foundation (http://www.marshmed.org/genetics/). Ambiguous ordering of markers in the Marshfield map was resolved using the program MULTIMAP (T. C. Matise et al., 1994, Nature Genet. 6:384-390).

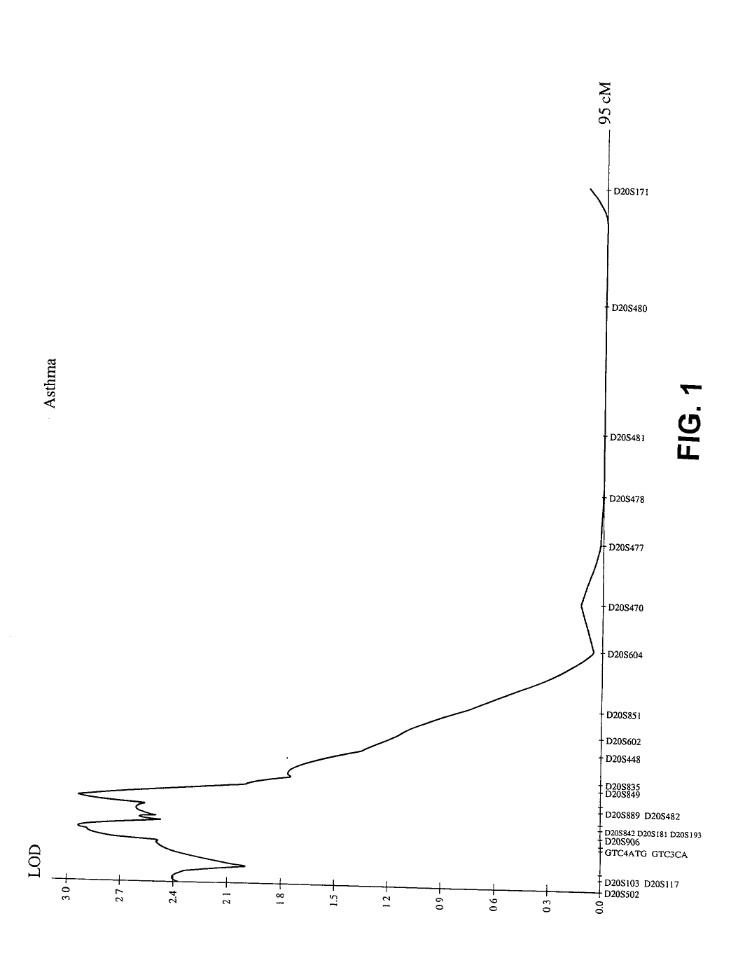

Using the discrete phenotype of asthma (yes/no), a candidate region was identified on chromosome 20 with a LOD score of 2.94, based on 462 nuclear families. FIG. 1 displays the multipoint LOD score against the map location of the markers along chromosome 20. A Maximum LOD Score (MLS) of 2.94 was obtained at location 7.9 cM, 0.3 cM proximal to marker D20S906. A second MLS of 2.94 was obtained at marker D20S482 at location 12.1 cM. An excess sharing by descent (Identity By Descent (IBD)=2) of 0.31 was observed at both maximum LOD scores. Table 2 lists the single and multipoint LOD scores at each marker. Analyses were done using a conservative approach by weighting multiple sibling pairs within a sibship. When affected sib pairs were utilized in the linkage analyses without weighting the LOD score on chromosome 20 maximized at D20S482 with a value of 3.19. Thus, these data provided strong evidence for the presence of an asthma susceptibility gene in this region of chromosome 20.

TABLE 2

| Marker | Distance | Single-point | Multipoint |
|---|---|---|---|
| D20S502 | 0.5 | 0.7 | 2.4 |
| D20S103 | 2.1 | 2.4 | 2.3 |
| D20S117 | 2.8 | 1.2 | 2.0 |
| GTC4ATG | 6.3 | 2.4 | 2.5 |
| GTC3CA | 6.6 | 1.3 | 2.7 |
| D20S906 | 7.6 | 2.9 | 2.9 |
| D20S842 | 9.0 | 1.3 | 2.5 |
| D20S181 | 9.5 | 1.8 | 2.6 |
| D20S193 | 9.5 | 2.5 | 2.5 |
| D20S889 | 11.2 | 1.6 | 2.6 |
| D20S482 | 12.1 | 1.9 | 2.9 |
| D20S849 | 14.0 | 0.8 | 2.0 |
| D20S835 | 15.1 | 0.5 | 1.8 |
| D20S448 | 18.8 | 1.4 | 1.4 |
| D20S602 | 21.2 | 1.1 | 1.1 |
| D20S851 | 24.7 | 1.0 | 0.8 |
| D20S604 | 32.9 | 0.0 | 0.1 |
| D20S470 | 39.3 | 0.0 | 0.1 |
| D20S477 | 47.5 | 0.0 | 0.0 |
| D20S478 | 54.1 | 0.0 | 0.0 |
| D20S481 | 62.3 | 0.0 | 0.0 |
| D20S480 | 79.9 | 0.0 | 0.0 |
| D20S171 | 95.7 | 0.4 | 0.1 |

2. Phenotypic Subgroups:

Nuclear families were ascertained by the presence of at least two affected siblings with a current physician's diagnosis of asthma, as well as the use of asthma medication. In the initial analysis (see above), the evidence was examined for linkage based on that dichotomous phenotype (asthma—yes/no). To further characterize the linkage signals, additional quantitative traits were measured in the clinical protocol. Since quantitative trait loci (QTL) analysis tools with correction for ascertainment was not available, the following approach was taken to refine the linkage and association analyses:

i. Phenotypic subgroups that could be indicative of an underlying genotypic heterogeneity were identified. Asthma subgroups were defined according to 1) bronchial hyper-responsiveness (BHR) to methacholine challenge; or 2) to atopic status using quantitative measures like total serum IgE and specific IgE to common allergens.

ii. Non-parametric linkage analyses were performed on subgroups to test for the presence of a more homogeneous sub-sample. If genetic heterogeneity was present in the sample, the amount of allele sharing among phenotypically similar siblings was expected to increase in the appropriate subgroup in comparison to the full sample. A narrower region of significant increased allele sharing was also expected to result unless the overall LOD score decreased as a consequence of having a smaller sample size and of using an approximate partitioning of the data.

iii. Alternatively, allele sharing probabilities were parameterized as a function of the quantitative trait value of each child in a given sib pair, as advocated by N. Morton and implemented in his program BETA (N. Morton, 1996, Proc. Natl. Acad. Sci. USA 93:3471-3476). This approach alleviated the need to dichotomize a quantitative trait. However, the program did not correct for the use of non-independent sib pairs in sibship of size 3 or larger. As such it did not provide an accurate measure of the significance of a linkage finding, but was used to corroborate the localization of the linkage signal.

3. Results for BHR and IgE:

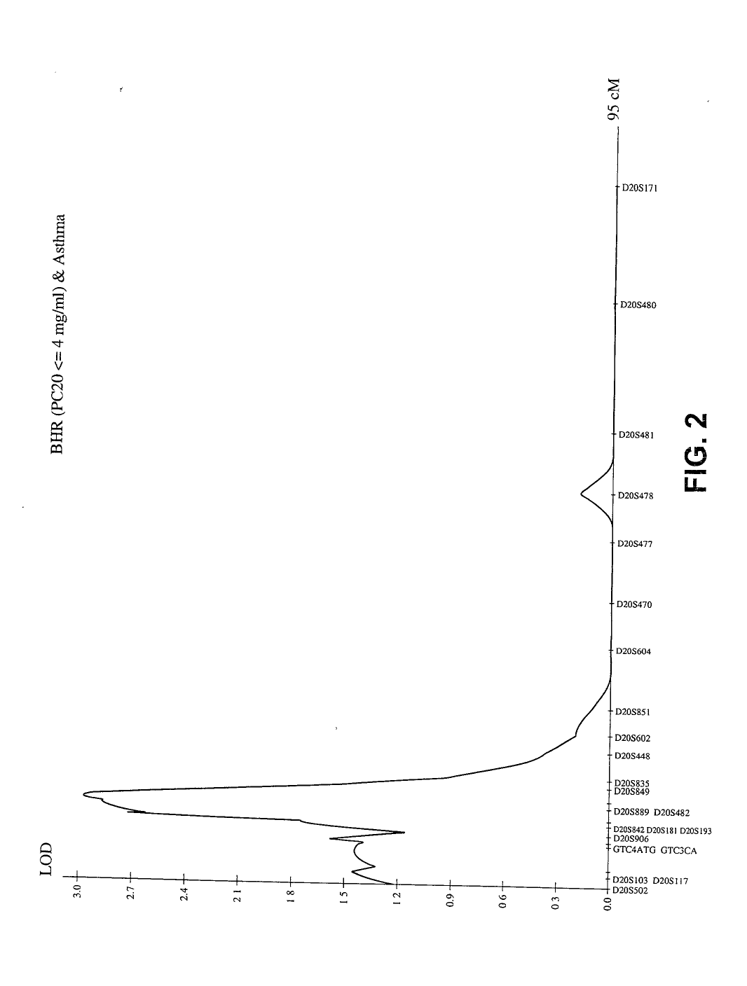

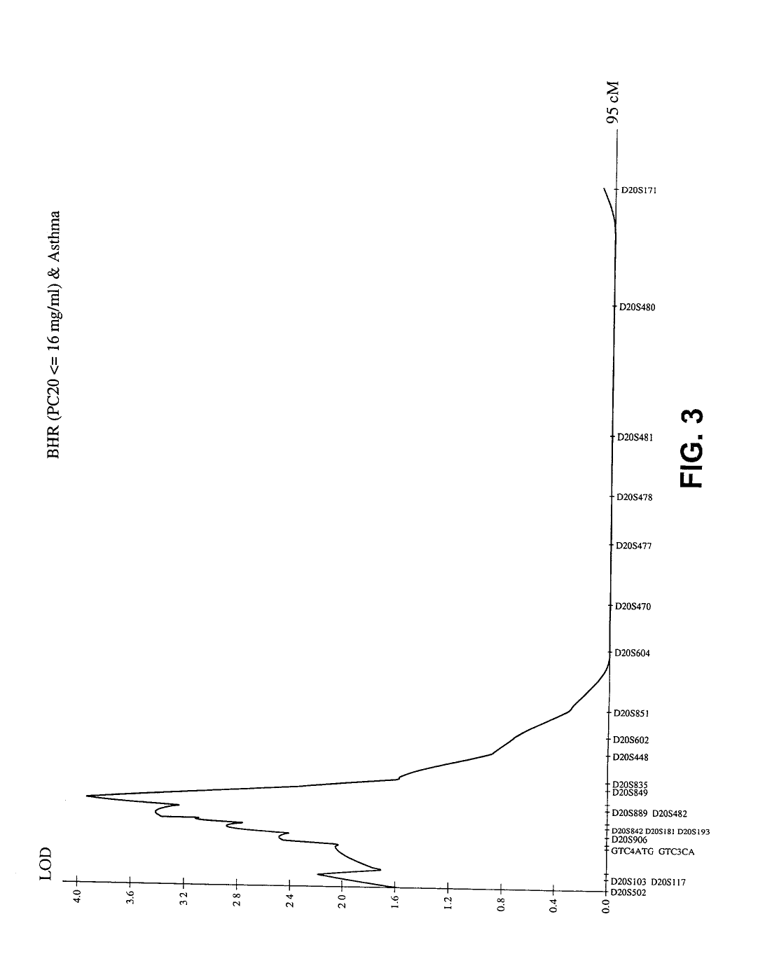

PC₂₀, the concentration of methacholine resulting in a 20% drop in FEV₁ (forced expiratory volume), was polychotomized in four groups and analyses were performed on the subsets of asthmatic children with mild to severe BHR(PC₂₀≦4 mg/ml) or PC₂₀(4), as well as on the broader subset with borderline to severe BHR(PC₂₀≦16 mg/ml) or PC20(16). As shown in the LOD plot in FIG. 2, the MLS for the subset of 127 nuclear families with at least two PC20(4) affected sibs was 2.97 at 11.8 cM, 0.3 cM from D20S482, with an excess sharing by descent of 0.37. As shown in FIG. 3, for the 218 nuclear families with at least two PC₂₀(16), the MLS was 3.93 at D20S482 with an excess sharing of 0.36. Both PC₂₀(4) and PC₂₀(16) strongly implicated the region of chromosome 20 under the second peak around marker D20S482. When considering the more extreme phenotype, PC₂₀(4), a higher proportion of families was linked to the region. However, the increase in LOD score for the PC₂₀(16) phenotype indicated that families concordant for the milder BHR phenotype also contributed to the linkage signal and would provide a larger pool of linked families.

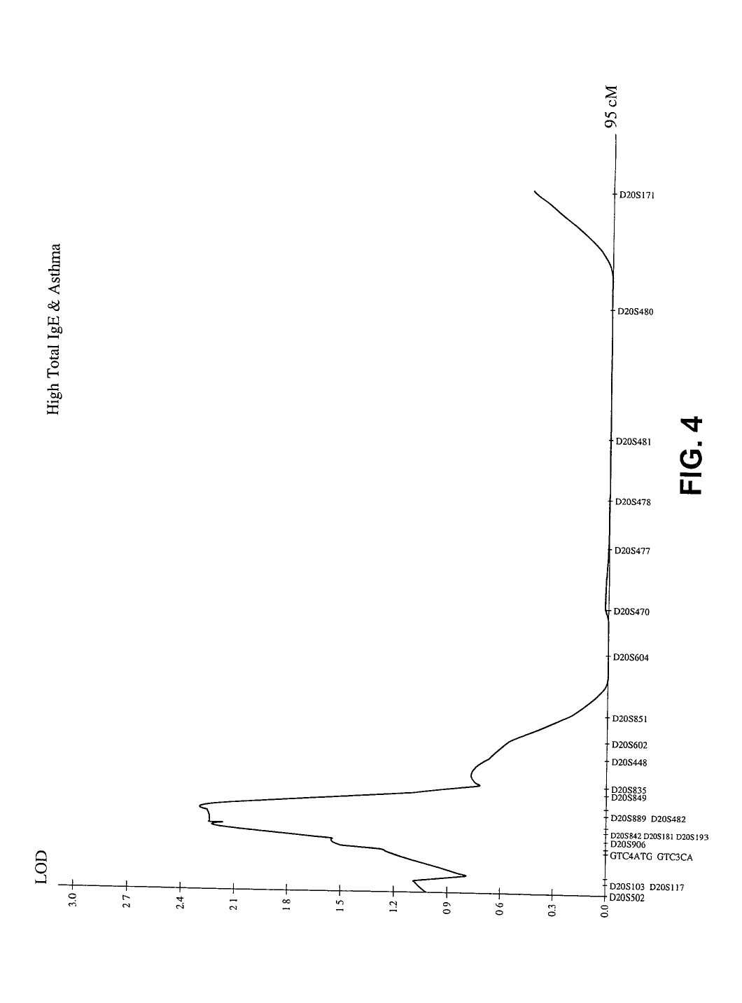

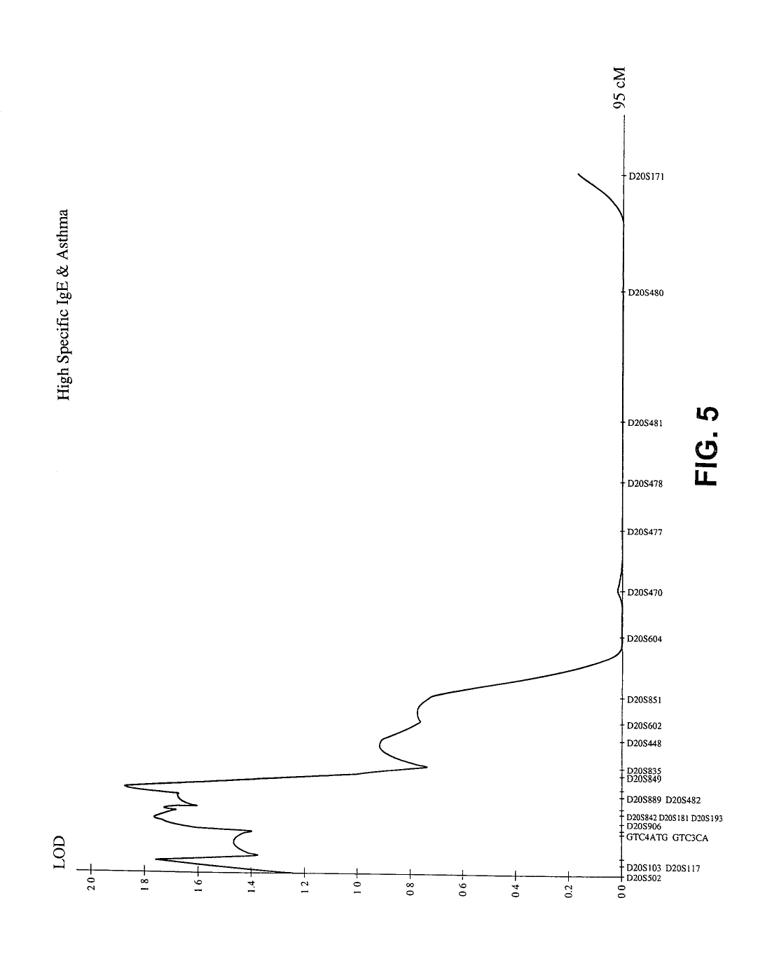

Total IgE was dichotomized using an age specific cutoff for elevated levels (one standard deviation above the mean). Similarly, a dichotomous variable was created using specific IgE to common allergens. An individual was assigned a high specific IgE value if his/her level was positive (grass or tree) or elevated (>0.35 KU/L for cat, dog, mite A, mite B, alternaria, or ragweed) for at least one such measure. In linkage analyses, the subset of asthmatic children with high total IgE (274 families) was given a maximum LOD score of 2.3 at 11.6 cM (FIG. 4), while the subset with high specific IgE (288 families) was given a LOD score of 1.87 at 12.1 cM (FIG. 5). Similar to the BHR results, analyses based on IgE implicated the region under the second peak around marker D20S482 The substantially lower LOD scores using the subset of affected sibs concordant for atopy indicated the presence of groups with fewer linked families. Thus, atopy in asthmatic individuals was not the primary phenotype associated with the linkage signal on chromosome 20.

The BETA program (Morton, 1996) was used on two scales for PC₂₀. Individuals that did not drop 20% by the last dose administered (16 mg/ml) were assigned an arbitrary value of 32 mg/ml. First, a (0,1)-severity scale was constructed by applying a linear transformation to PC₂₀ where 0 mg/ml received a score of 1 and 32 mg/ml received a score of 0. For this scale, individuals that did not drop 20% in their FEV₁ did not contribute to the LOD score. A maximum LOD score of 3.43 was achieved at 12.1 cM with marker D20S482. Second, a linear transformation of PC₂₀ was used where 0 mg/ml received a score of 1 and 32 mg/ml a score of −1. In other words, in addition to the high concordant pairs, discordant pairs and concordant pairs that did not drop would also contribute to the LOD score. In contrast, individuals with PC₂₀ close to 16 mg/ml would have little impact on the LOD score. A maximum LOD score of 2.08 was again achieved at 12.1 cM.

Accordingly, a consistent pattern of evidence by linkage analysis pointed to the existence of an asthma susceptibility locus in the vicinity of marker D20S482. This was supported by the initial analysis of the asthma (yes/no) phenotype and by analyses of BHR in asthmatic individuals. Localization in the region of marker D20S482 was obtained using both BHR and IgE phenotypes.

### EXAMPLE 4

### Physical Mapping

The linkage results for chromosome 20 described above were used to delineate a candidate region for a disorder-associated gene located on chromosome 20. Gene discovery efforts were thus initiated in a 25 cM interval from the 20p telomere (marker D20S502) to marker D20S851, representing a >98% confidence interval. All genes known to map to this interval were considered as candidates. Intensive physical mapping (BAC contig construction) focused on a 90% confidence interval between markers D20S103 and D20S916, a 15 cM interval. The discovery of novel genes using direct cDNA selection focused on a 95% confidence interval between markers D20S502 (20p telomere) and D20S916, a 17 cM region.

The following section describes details of the efforts to generate cloned coverage of the disorder gene region on chromosome 20, i.e., construction of a BAC contig spanning the region. There were two primary reasons for using this approach: 1) to provide genomic clones for DNA sequencing (analysis of this sequence would provide information about the gene content of the region); and 2) to provide reagents for direct cDNA selection (this would provide additional information about novel genes mapping to the interval). The physical map consisted of an ordered set of molecular landmarks, and a set of bacterial artificial chromosome clones (BACs; U.-J. Kim et al., 1996, Genomics 34:213-218; H. Shizuya et al., 1992, Proc. Natl. Acad. Sci. USA 89:8794-8797) that contained the disorder gene region from human chromosome 20p13-p12.

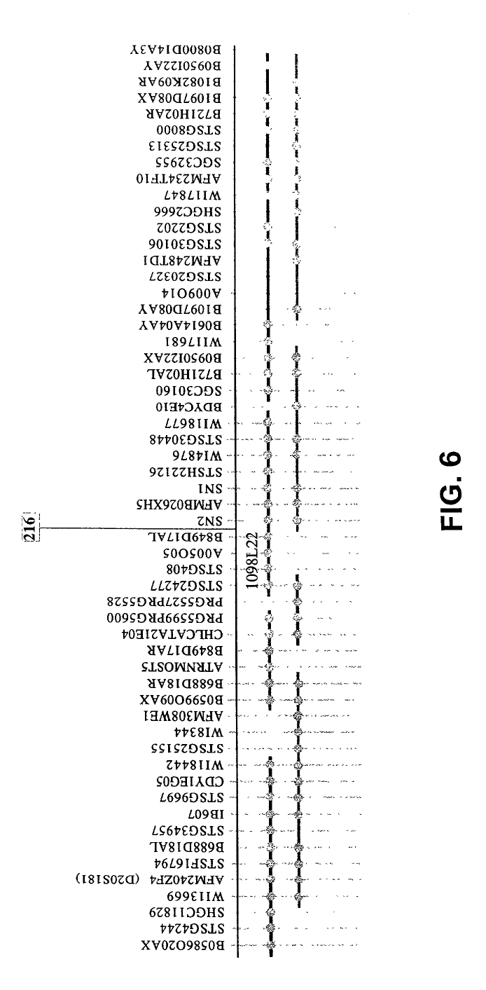

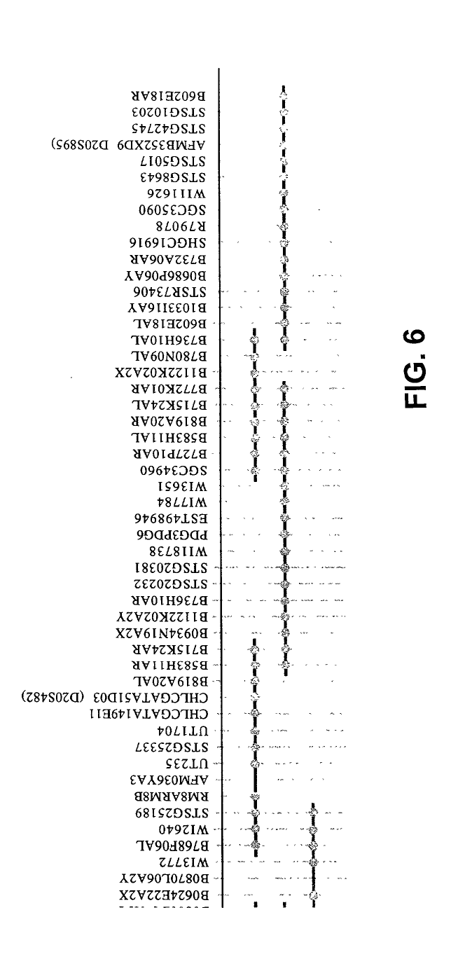

FIG. 6 depicts the BAC/STS content contig map of human chromosome 20p13-p12. Markers used to screen the RPCl-11 BAC library (P. dejong, Roswell Park Cancer Institute (RPCl)) are shown in the top row. Markers that were present in the Genome Database (GDB, http://gdbwww.gdb.org/) are represented by GDB nomenclature. The BAC clones are shown below the markers as horizontal lines. BAC RPCl-11_{—}1098L22 is labeled and the location of Gene 216, described herein, is indicated at the top of the figure.

1. Map Integration.

Various publicly available mapping resources were utilized to identify existing STS (sequence tagged site) markers (Olson et al., 1989, Science, 245:1434-1435) in the 20p13-p12 region. Resources included the GDB (http:/gdbwww.gdb.org/), Genethon (http://www.genethon. fr/genethon_en.html), Marshfield Center for Medical Genetics (http://www.marshmed.org/genetics/), the Whitehead Institute Genome Center (http://www-genome.wi.mit.edu/), GeneMap98, dbSTS and dbEST (NCBI, http://www.ncbi.nim.nih.gov/), the Sanger Centre (http://www.sanger.ac.uk/), and the Stanford Human Genome Center (http://www-shgc.stanford.edul). Maps were integrated manually to identify markers mapping to the disorder region. A list of the markers is provided in Table 3.

2. Marker Development:

Sequences for existing STSs were obtained from the GDB, RHDB (http://www.ebi.ac.uk/RHdb/), or NCBI, and were used to pick primer pairs (overgos; see Table 3) for BAC library screening. Novel markers were developed either from publicly available genomic sequences, proprietary cDNA sequences, or from sequences derived from BAC insert ends (described below). Primers were chosen using a script that automatically performs vector and repetitive sequence masking using CROSSMATCH (P. Green, University of Washington). Subsequent primer selection was performed using a customized Filemaker Pro database (http://www.filemaker.com). Primers for use in PCR-based clone confirmation or radiation hybrid mapping (described below) were chosen using the program Primer3 (Steve Rozen, Helen J. Skaletsky, 1996, 1997, http://www-genome.wi.mit.edulgenome_software/other/primer3.html).

TABLE 3

| | | | | | SEQ | | SEQ |
|---|---|---|---|---|---|---|---|
| | | DNA | | | ID | ID | |
| Overgo | Locus | Type | Gene | Forward Primer | NO | Reverse Primer | NO |
| stSG24277 | | Genomic | | aactcttgaaatgagaagcgtg | 34 | aaccaccacggattcacgcttc | 45 |
| stSG4O8 | | EST | | aatatcatgcaccatgacccac | 35 | ataaccagatggctgtgggtca | 46 |
| A005O05 | | EST | Attractin (ATTN) | tggagtaagtattgtaaactat | 36 | atccccgcaatgaaatagttta | 47 |
| B849D17AL | | BACend | | ggagcttatcctggattatcta | 37 | gttgagagcccacttagataat | 48 |
| SN2 | | EST | Sialoadhesin (SN) | agagccacacatccatgtcctg | 38 | gcattgggggaagccaggacat | 49 |
| AFMb026xh5 | D205867 | MSAT | | aagccactctgtgaattgccat | 39 | gccactaggaggcaatggcaat | 50 |
| SN1 | | EST | Sialoadhesin (SN) | gagtagtcgtagtaccagatgg | 40 | cgacggcatcacggccatctgg | 51 |
| stsH22126 | | EST | | gtctggcaatggagcatgaaaa | 41 | tccaggctcattcattttcatg | 52 |
| WI4876 | D20S752 | Genomic | attagagcacatgaaggaaagg | 42 | tgacatcaacttctcctttcct | 53 | |
| stSG30448 | | EST | | acactgctttgggggacaggct | 43 | agttgcagagacctagcctgtc | 54 |
| WI18677 | | EST | cacgacgccacagagccagctc | 44 | tctgggagaggacggagctggc | 55 | |

3. Radiation Hybrid (RH) Mapping:

Radiation hybrid mapping was performed against the Genebridge4 panel (Gyapay et al., 1996, Hum. Mol. Genet. 5:339-46) purchased from Research Genetics, in order to refine the chromosomal localization of genetic markers used in genotyping as well as to identify, confirm, and refine localizations of markers from proprietary sequences. Standard PCR procedures were used for typing the RH panel with markers of interest. Briefly, 10 μl PCR reactions contained 25 ng DNA of each of the 93 Genebridge4 RH samples. PCR products were electrophoresed on 2% agarose gels (Sigma) containing 0.5 μg/ml ethidium bromide in 1× TBE at 150 volts for 45 min. Model A3-1 electrophoresis systems were used (Owl Scientific Products, Portsmouth, N.H.). Typically, gels contained 10 tiers of lanes with 50 wells/tier. Molecular weight markers (100 bp ladder, GibcoBRL, Rockville, Md.) were loaded at both ends of the gel. Images of the gels were captured with a Kodak DC40 CCD camera and processed with Kodak 1D software (www.kodak.com). The gel data were exported as tab delimited text files; names of the files included information about the panel screened, the gel image files and the marker screened. These data were automatically imported using a customized Perl script into Filemaker databases for data storage and analysis. The data were then automatically formatted and submitted to an internal server for linkage analysis to create a radiation hybrid map using RHMAPPER (L. Stein et al., 1995; available from Whitehead Institute/MIT Center for Genome Research, at http://www.genome.wi.mit.edu/ftp/pub/software/rhmapper/, and via anonymous ftp to ftp.genome.wi.mit.edu, in the directory /pub/software/rhmapper.)

4. BAC Library Screening:

The protocol used for BAC library screening was based on the “overgo” method, originally developed by John McPherson at Washington University in St. Louis (http://www.tree.caltech.edu /protocols/overgo.html, and W -W. Cai et al., 1998, Genomics 54:387-397). This method involved filling in the overhangs generated after annealing two primers, each 22 nucleotides in length, which overlap by 8 nucleotides. The resulting labeled 36 bp product was then used in hybridization-based screening of high density grids derived from the RPCI-11 BAC library (dejong, supra). Typically, 15 probes were pooled together to hybridize 12 filters (13.5 genome equivalents).

Stock solutions (2 μM) of combined complementary oligos were heated at 80° C. for 5 min, placed at 37° C. for 10 min, and then stored on ice. Labeling reactions included the following: 1.0 μl H₂O; 5 μl mixed oligos (2 μM each); 0.5 μl BSA (2 mg/ml); 2 μl OLB (-A, -C, -N6) Solution (see below); 0.5 μl ³²P-dATP (3000 Ci/mmol); 0.5 μl ³²P-dCTP (3000 Ci/mmol); and 0.5 μl Klenow fragment (5 U/μl). The reaction was incubated at room temperature for 1 hr, and unincorporated nucleotides were removed using Sephadex G50 spin columns. Solution O: 1.25 M Tris-HCL, pH 8, 125 M MgCl₂; Solution A: 1 ml Solution O, 18 μl 2-mercaptoethanol, 5 μl 0.1M dTTP, 5 μl 0.1 M dGTP; Solution B: 2 M HEPES-NaOH, pH 6.6; Solution C: 3 mM Tris-HCl, pH 7.4, 0.2 mM EDTA; Solutions A, B, and C were combined to a final ratio of 1:2.5:1.5, and aliquots were stored at −20° C.

High-density BAC library membranes were pre-wetted in 2× SSC at 58° C. Filters were then drained slightly and placed in hybridization solution (1% BSA; 1 mM EDTA, pH 8.0; 7% SDS; and 0.5 M sodium phosphate), pre-warmed to 58° C., and incubated at 58° C. for 2-4 hr. Typically, 6 filters were hybridized in each container. Ten milliliters of pre-hybridization solution was removed, combined with the denatured overgo probes, and added back to the filters. Hybridization was performed overnight at 58° C. The hybridization solution was removed and filters were washed once in 2× SSC, 0.1% SDS, followed by a 30 min wash in the same solution at 58° C. Filters were then washed in: 1) 1.5× SSC and 0.1% SDS at 58° C. for 30 min; 2) 0.5× SSC and 0.1% SDS at 58° C. for 30 min; and finally in 3) 0.1× SSC and 0.1% SDS at 58° C. for 30 min. Filters were then wrapped in Saran Wrap and exposed to film overnight. To remove bound probe, filters were treated in 0.1× SSC and 0.1% SDS pre-warmed to 95° C. and cooled room temperature. Clone addresses were determined as described by instructions supplied by RPCI.

To recover clonal BAC cultures from the library, a sample from the appropriate library well was plated by streaking onto LB agar (T. Maniatis et al., 1982, Molecular Cloning: A Laboratory Manual, Cold Spring Harbor Laboratory, Cold Spring Harbor, N.Y.) containing 12.5 μg/ml chloramphenicol (Sigma), and plates were incubated overnight. A single colony and a portion of the initial streak quadrant were inoculated into 400 μl LB plus chloramphenicol in wells of a 96 well plate. Cultures were grown overnight at 37° C. For storage, 100 μl of 80% glycerol was added and the plates placed at −80° C.

To determine the marker content of clones, aliquots of the 96 well plate cultures were transferred to the surface of nylon filters (GeneScreen Plus, NEN) placed on LB/chloramphenicol Petri plates. Colonies were grown overnight at 37° C. and colony lysis was performed by placing filters on pools of: 1) 10% SDS for 3 min; 2) 0.5 N NaOH and 1.5 M NaCl for 5 min; and 3) 0.5 M Tris-HCl, pH 7.5, and 1 M NaCl for 5 min. Filters were then air-dried and washed free of debris in 2× SSC for 1 hr. The filters were air-dried for at least 1 hr and DNA was crosslinked linked to the membrane using standard conditions. Probe hybridization and filter washing were performed as described above for the primary library screening. Confirmed clones were stored in LB containing 15% glycerol.

In certain cases, polymerase chain reaction (PCR) was used to confirm the marker content of clones. PCR conditions for each primer pair were initially optimized with respect to MgCl₂ concentration. The standard buffer was 10 mM Tris-HCl (pH 8.3), 50 mM KCl, MgCl₂, 0.2 mM each dNTP, 0.2 μM each primer, 2.7 ng/μl human DNA, 0.25 units of AmpliTaq (Perkin Elmer) and MgCl₂ concentrations of 1.0 mM, 1.5 mM, 2.0 mM or 2.4 mM. Cycling conditions included an initial denaturation at 94° C. for 2 min followed by 40 cycles at 94° C. for 15 sec, 55° C. for 25 sec, and 72° C. for 25 sec followed by a final extension at 72° C. for 3 min. Depending on the results from the initial round of optimization the conditions were further optimized if necessary. Variables included increasing the annealing temperature to 58° C. or 60° C., increasing the cycle number to 42 and the annealing and extension times to 30 sec, and using AmpliTaqGold (Perkin Elmer).

5. BAC DNA Preparation:

Several different types of DNA preparation methods were used for isolation of BAC DNA. The manual alkaline lysis miniprep protocol listed below (Maniatis et al., 1982) was successfully used for most applications, i.e., restriction mapping, CHEF gel analysis and FISH mapping, but was not reproducibly successful in endsequencing. The Autogen protocol described below was used specifically for BAC DNA preparation for endsequencing.

For manual alkaline lysis BAC minipreps, bacteria were grown in 15 ml terrific broth (TB) containing 12.5 μg/ml chloramphenicol. Cultures were placed in a 50 ml conical tube at 37° C. for 20 hr with shaking at 300 rpm. The cultures were centrifuged in a Sorvall RT 6000 D at 3000 rpm (1800× g) at 4° C. for 15 min. The supernatant was then aspirated as completely as possible. In some cases cell pellets were frozen at −20° C. at this step for up to 2 weeks. The pellet was then vortexed to homogenize the cells and minimize clumping. Following this, 250 μl of P1 solution (50 mM glucose, 15 mM Tris-HCl, pH 8, 10 mM EDTA, and 100 μg/ml RNase A) was added and the mixture pipetted up and down to mix. The mixture was then transferred to a 2 ml Eppendorf tube. Subsequently, 350 μl of P2 solution (0.2 N NaOH, 1% SDS) was added, mixed gently, and the mixture was incubated for 5 min at room temperature. Then, 350 μl of P3 solution (3 M KOAc, pH 5.5) was added and mixed gently until a white precipitate formed. The solution was incubated on ice for 5 min and then centrifuged at 4° C. in a microfuge for 10 min.

The supernatant was transferred carefully (avoiding the white precipitate) to a fresh 2 ml Eppendorf tube, and 0.9 ml of isopropanol was added; the solution was mixed and left on ice for 5 min. The samples were centrifuged for 10 min, and the supernatant removed carefully. Pellets were washed in 70% ethanol and air-dried for 5 min. Pellets were resuspended in 200 μl of TE8 (10 mM Tris-HCl, pH 8.0, 1.0 mM EDTA, pH 8.0), and RNase (Boehringer Mannheim, http://biochem.boehringer-mannheim.com) added to 100 μg/ml. Samples were incubated at 37° C. for 30 min, then precipitated by addition of NH₄OAc to 0.5 M and 2 volumes of ethanol. Samples were centrifuged for 10 min, and the pellets were washed with 70% ethanol. The pellets were air-dried and dissolved in 50 μl TE8. Typical yields for this DNA prep were 3-5 μg per 15 ml bacterial culture. Ten to 15 μl of DNA was used for EcoRI restriction analysis; 5 μl was used for NotI digestion and clone insert sizing by CHEF gel electrophoresis.

Autogen 740 BAC DNA preparations for endsequencing were made by dispensing 3 ml of LB media containing 12.5 μg/ml of chloramphenicol into autoclaved Autogen tubes. A single tube was used for each clone. For inoculation, glycerol stocks were removed from −70° C. storage and placed on dry ice. A small portion of the glycerol stock was removed from the original tube with a sterile toothpick and transferred into the Autogen tube. The toothpick was left in the Autogen tube for at least two min before discarding. After inoculation the tubes were covered with tape to ensure that the seal was tight. When all samples were inoculated, the tubes were transferred into an Autogen rack holder and placed into a rotary shaker. Cultures were incubated at 37° C. for 16-17 hr at 250 rpm. Following this, standard conditions for BAC DNA preparation, as defined by the manufacturer, were used to program the Autogen. However, samples were not dissolved in TE8 as part of the program. DNA pellets were left dry.

When the program was completed, the tubes were removed from the output tray and 30 μl of sterile distilled and deionized H₂O was added directly to the bottom of the tube. The tubes were then gently shaken for 2-5 sec and then covered with parafilm and incubated at room temperature for 1-3 hr. DNA samples were then transferred to an Eppendorf tube and used either directly for sequencing or stored at 4° C. for later use.

6. BAC Clone Characterization:

DNA samples prepared either by manual alkaline lysis or the Autogen protocol were digested with EcoRI for analysis of restriction fragment sizes. These data were used to compare the extent of overlap among clones. Typically 1-2 μg were used for each reaction. Reaction mixtures included: 1× Buffer 2 (NEB); 0.1 mg/ml BSA (NEB); 50 μg/ml RNase A (Boehringer Mannheim); and 20 units of EcoRI (NEB) in a final volume of 25 μl. Digestions were incubated at 37° C. for 4-6 hr. BAC DNA was also digested with NotI for estimation of insert size by CHEF gel analysis (see below). Reaction conditions were identical to those for EcoRI, except that 20 units of NotI were used. Six microliters of 6× Ficoll loading buffer containing bromphenol blue and xylene cyanol was added prior to electrophoresis.

EcoRI digests were analyzed on 0.6% agarose (Seakem, FMC Bioproducts, Rockland, Me.) in 1× TBE containing 0.5 μg/ml ethidium bromide. Gels (20 cm×25 cm) were electrophoresed in a Model A4 electrophoresis unit (Owl Scientific) at 50 volts for 20-24 hr. Molecular weight size markers included undigested lambda DNA, HindIII digested lambda DNA, and HaeIII digested .X174 DNA. Molecular weight markers were heated at 65° C. for 2 min prior to loading the gel. Images were captured with a Kodak DC40 CCD camera and analyzed with Kodak 1 D software.

NotI digests were analyzed on a CHEF DRII (Bio-Rad) electrophoresis unit according to the manufacturer's recommendations. Briefly, 1% agarose gels (Bio-Rad pulsed field grade) were prepared in 0.5× TBE, equilibrated for 30 min in the electrophoresis unit at 14° C., and electrophoresed at 6 volts/cm for 14 hr with circulation. Switching times were ramped from 10 sec to 20 sec. Gels were stained after electrophoresis in 0.5 μg/ml ethidium bromide. Molecular weight markers included undigested lambda DNA, HindIII digested lambda DNA, lambda ladder PFG ladder, and low range PFG marker (all from NEB).

7. BAC Endsequencing:

The sequence of BAC insert ends utilized DNA prepared by either of the two methods described above. The ends of BAC clones were sequenced for the purpose of filling gaps in the physical map and for gene discovery information. The following vector primers specific to the BAC vector pBACe3.6 were used to generate endsequence from BAC clones: pBAC 5′-2 (TGT AGG ACT ATA TTG CTC; SEQ ID NO:56) and pBAC 3′-1 (CGA CAT TTA GGT GAC ACT; SEQ ID NO:57).

The ABI dye-terminator sequencing protocol was used to set up sequencing reactions for 96 clones. The BigDye (ABI; PE Applied Biosystems) Terminator Ready Reaction Mix with AmpliTaq″ FS, Part number 4303151, was used for sequencing with fluorescently labeled dideoxy nucleotides. A master sequencing mix was prepared for each primer reaction set including: 1600 μl of BigDye terminator mix (ABI; PE Applied Biosystems); 800 μl of 5× CSA buffer (ABI; PE Applied Biosystems); 800 μl of primer (either pBAC 5′-2 or pBAC 3′-1 at 3.2 μM). The sequencing cocktail was vortexed to ensure it was well-mixed and 32 μl was aliquoted into each PCR tube. Eight microliters of the Autogen DNA for each clone was transferred from the DNA source plate to a corresponding well of the PCR plate. The PCR plates were sealed tightly and centrifuged briefly to collect all the reagents. Cycling conditions were as follows: 1) 95° C. for 5 min; 2) 95° C. for 30 sec; 3) 50° C. for 20 sec; 4) 65° C. for 4 min; 5) steps 2 through 4 were repeated 74 times; and 6) samples were stored at 4° C.

At the end of the sequencing reaction, the plates were removed from the thermocycler and centrifuged briefly. Centri•Sep 96 plates were then used according to manufacturer's recommendations to remove unincorporated nucleotides, salts, and excess primers. Each sample was resuspended in 1.5 μl of loading dye, and 1.3 μl of the mixture was loaded on ABI 377 Fluorescent Sequencers. The resulting endsequences were then used to develop markers to rescreen the BAC library for filling gaps and were also analyzed by BLASTN searching for EST or gene content.

### EXAMPLE 5

### Subcloning and Sequencing of BAC RPCI-11 1098L22

The physical map of the chromosome 20 region provided the location of the BAC RPCI-11_{—}1098L22 clone that contains Gene 216 (see FIG. 6). The BAC RPCI-11_{—}1098L22 clone was deposited as clone RP11-1098L22 with the American Type Culture Collection (ATCC), 10801 University Blvd., Manassas, Va. 20110-2209 USA, under ATCC Designation No. PTA-3171, on Mar. 14, 2001 according to the terms of the Budapest Treaty. DNA sequencing of BAC, RPCI-11-1098L22 from the region was completed. BAC RPCI-11-1098L22 DNA, (the “BAC DNA”) was isolated according to one of two protocols: either a QIAGEN purification (QIAGEN, Inc., Valencia, Calif., per manufacturer's instructions) or a manual purification using a method which was a modification of the standard alkaline lysis/Cesium Chloride preparation of plasmid DNA (see e.g., F. M. Ausubel et al., 1997, Current Protocols in Molecular Biology, John Wiley & Sons, New York, N.Y.). Briefly, for the manual protocol, cells were pelleted, resuspended in GTE (50 mM glucose, 25 mM Tris-Cl (pH 8), 10 mM EDTA) and lysozyme (50 mg/ml solution), followed by addition of NaOH/SDS (1% SDS and 0.2N NaOH) and then an ice-cold solution of 3M KOAc (pH 4.5-4.8). RnaseA was added to the filtered supernatant, followed by treatment with Proteinase K and 20% SDS. The DNA was then precipitated with isopropanol, dried, and resuspended in TE (10 mM Tris, 1 mM EDTA (pH 8.0)). The BAC DNA was further purified by cesium chloride density gradient centrifugation (Ausubel et al., 1997).

Following isolation, the BAC DNA was hydrodynamically sheared using HPLC (Hengen et al., 1997, Trends in Biochem. Sci., 22:273-274) to an insert size of 2000-3000 bp. After shearing, the DNA was concentrated and separated on a standard 1% agarose gel. A single fraction, corresponding to the approximate size, was excised from the gel and purified by electroelution (Sambrook et al., 1989).

The purified DNA fragments were then blunt-ended using T4 DNA polymerase. The blunt-ended DNA was then ligated to unique BstXl-linker adapters (5′ GTCTTCACCACGGGG (SEQ ID NO:58) and 5′ GTGGTGAAGAC (SEQ ID NO:59) in 100-1000 fold molar excess). These linkers were complimentary to the BstXl-cut pMPX vectors, while the overhang was not self-complimentary. Therefore, the linkers would not concatemerize, nor would the cut-vector re-ligate to itself easily. The linker-adapted inserts were separated from unincorporated linkers on a 1% agarose gel and purified using GeneClean (BIO 101, Inc., Vista, Calif.). The linker-adapted insert was then ligated to a modified pBlueScript vector to construct a “shotgun” subclone library. The vector contained an out-of-frame lacZ gene at the cloning site, which became in-frame in the event that an adapter-dimer was cloned. Such adapter-dimer clones gave rise to blue colonies, which were avoided.

All subsequent steps were based on sequencing by ABI377 automated DNA sequencing methods. Major modifications to the protocols are highlighted below. Briefly, the library was transformed into DH5-competent cells (GibcoBRL, DH5-transformation protocol). Transformed cells were plated onto antibiotic plates containing ampicillin and IPTG/X-gal. The plates were incubated overnight at 37° C. White colonies were identified and then used to plate individual clones for sequencing. The cultures were grown overnight at 37° C. DNA was purified using a silica bead DNA preparation method (Ng et al., 1996, Nucl. Acids Res., 24:5045-5047). In this manner, 25 μg of DNA was obtained per clone.

These purified DNA samples were sequenced using ABI dye-terminator chemistry. The ABI dye terminator sequence reads were run on ABI377 machines and the data were directly transferred to UNIX machines following lane tracking of the gels. All reads were assembled using PHRAP (P. Green, Abstracts of DOE Human Genome Program Contractor-Grantee Workshop V, January 1996, p.157) with default parameters and quality scores. The assembly was done at 8-fold coverage and yielded 1 contig, BAC RPCI-11-1098L22. SEQ ID NO:5 (FIG. 7) comprises a portion of the BAC that includes the genomic sequence of Gene 216.

### EXAMPLE 6

### Gene Identification

Any gene or EST mapping to the interval based on public map data or proprietary map data was considered a candidate respiratory disease gene. Public map data were derived from several sources: the Genome Database (GDB, http:/gdbwww.gdb.org/), the Whitehead Institute Genome Center (http://www-genome.wi.mit.edu/), GeneMap98, UniGene, OMIM, dbSTS and dbEST (NCBI, http://www.ncbi.nlm.nih.gov/), the Sanger Centre (http://www.sanger.ac.uk/), and the Stanford Human Genome Center (http://www-shgc.stanford.edu/). Proprietary data was obtained from sequencing genomic DNA (cloned into BACs) or cDNAs (identified by direct selection, screening of cDNA libraries or full length sequencing of IMAGE Consortium (http://www-bio.11nl.gov/bbrp/image.html) cDNA clones).

1. Gene Identification from Clustered DNA Fragments.

DNA sequences corresponding to gene fragments in public databases (GenBank and human dbEST) and proprietary cDNA sequences (IMAGE consortium and direct selected cDNAs) were masked for repetitive sequences and clustered using the PANGEA Systems (Oakland, Calif.) EST clustering tool. The clustered sequences were then subjected to computational analysis to identify regions bearing similarity to known genes. This protocol included the following steps:

a. The clustered sequences were compared to the publicly available UniGene database (NCBI) using the BLASTN2 algorithm (Altschul et al., 1997). The parameters for this search were: E=0.05, v=50, B=50, where E was the expected probability score cutoff, V was the number of database entries returned in the reporting of the results, and B was the number of sequence alignments returned in the reporting of the results (Altschul et al., 1990).

b. The clustered sequences were compared to the GenBank database (NCBI) using BLASTN2 (Altschul et al., 1997). The parameters for this search were E=0.05, V=50, B=50, where E, V, and B were defined as above.

c. The clustered sequences were translated into protein sequences for all six reading frames, and the protein sequences were compared to a non-redundant protein database compiled from GenPept Swissprot PIR (NCBI). The parameters for this search were E=0.05, V=50, B=50, where E, V, and B were defined as above.

d. The clustered sequences were compared to BAC sequences (see below) using BLASTN2 (Altschul et al., 1997). The parameters for this search were E=0.05, V=50, B=50, where E, V, and B were defined as above.

2. Gene Identification from BAC Genomic Sequence:

Following assembly of the BAC sequences into contigs, the contigs were subjected to computational analyses to identify coding regions and regions bearing DNA sequence similarity to known genes. This protocol included the following steps:

a. Contigs were Degapped.

The sequence contigs often contained symbols (denoted by a period symbol) that represented locations where the individual ABI sequence reads had insertions or deletions. Prior to automated computational analysis of the contigs, the periods were removed. The original data were maintained for future reference.

b. BAC vector sequences were “masked” within the sequence by using the program crossmatch (P. Green, http:\\chimera.biotech.washington. edu\UWGC). Since the shotgun library construction detailed above left some BAC vector in the shotgun libraries, this program was used to compare the sequence of the BAC contigs to the BAC vector and to mask any vector sequence prior to subsequent steps. Masked sequences were marked by “X” in the sequence files, and remained inert during subsequent analyses.

c. E. coli sequences contaminating the BAC sequences were masked by comparing the BAC contigs to the entire E. coli DNA sequence.

d. Repetitive elements known to be common in the human genome were masked using CROSSMATCH(P. Green, University of Washington). In this implementation of crossmatch, the BAC sequence was compared to a database of human repetitive elements (J. Jerka, Genetic Information Research Institute, Palo Alto, Calif.). The masked repeats were marked by “X” and remained inert during subsequent analyses.

e. The location of exons within the sequence was predicted using the MZEF computer program (Zhang, 1997, Proc. Nat. Acad. Sci., 94:565-568)and GenScan gene prediction program (Burge and Karlin, J. Mol. Biol., 268:78-94).

f. The sequence was compared to the publicly available UniGene database (NCBI) using the BLASTN2 algorithm (Altschul et al., 1997). The parameters for this search were: E=0.05, v=50, B=50, where E was the expected probability score cutoff, V was the number of database entries returned in the reporting of the results, and B was the number of sequence alignments returned in the reporting of the results (Altschul et al., 1990).

g. The sequence was translated into protein sequences for all six reading frames, and the protein sequences were compared to a non-redundant protein database compiled from GenPept Swissprot PIR (NCBI). The parameters for this search were E=0.05, V=50, B=50, where E, V, and B were defined as above.

h. The BAC DNA sequence was compared to a database of clustered sequences using the BLASTN2 algorithm (Altschul et al., 1997). The parameters for this search were E=0.05, V=50, B=50, where E, V, and B were defined as above. The database of clustered sequences was prepared utilizing a proprietary clustering technology (PANGEA Systems, Inc.) using cDNA clones derived from direct selection experiments (described below), human dbEST sequences mapping to the 20p13-p12 region, proprietary cDNAs, GenBank genes, and IMAGE consortium cDNA clones.

i. The BAC sequence was compared to the sequences derived from the ends of BACs from the region on chromosomes 20 using the BLASTN2 algorithm (Altschul et al., 1997). The parameters for this search were E=0.05, V=50, B=50, where E, V, and B were defined as above. j. The BAC sequence was compared to the GenBank database (NCBI) using the BLASTN2 algorithm (Altschul et al., 1997). The parameters for this search were E=0.05, V=50, B=50, where E, V, and B were defined as above.

k. The BAC sequence was compared to the STS division of GenBank database (NCBI) using the BLASTN2 algorithm (Altschul et al., 1997). The parameters for this search were E=0.05, V=50, B=50, where E, V, and B were defined as above.

l. The BAC sequence was compared to the Expressed Sequence Tag (EST) GenBank database (NCBI) using the BLASTN2 algorithm (Altschul et al., 1997). The parameters for this search were E=0.05, V=50, B=50, where E, V, and B were defined as above.

c. Mapping Analysis

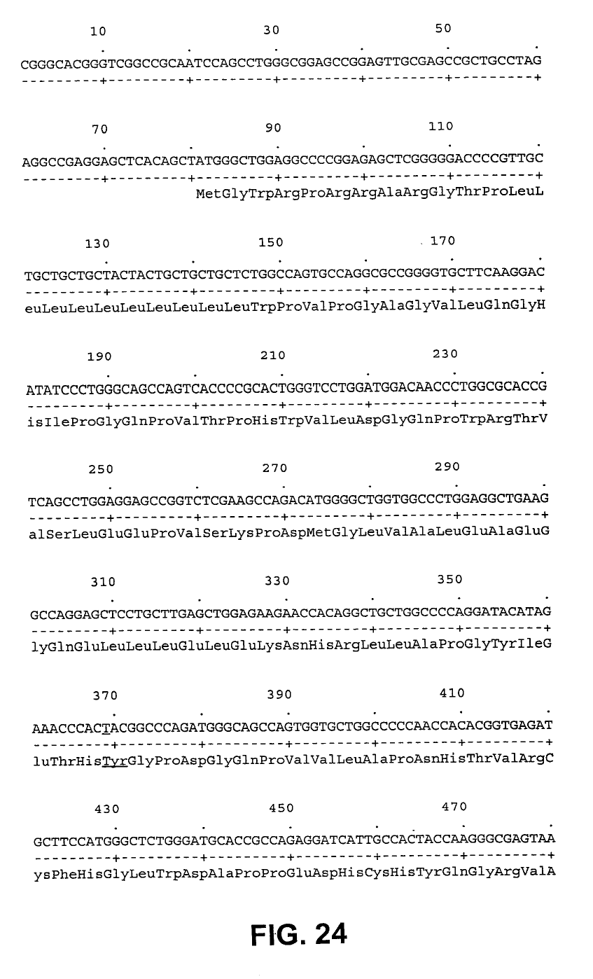

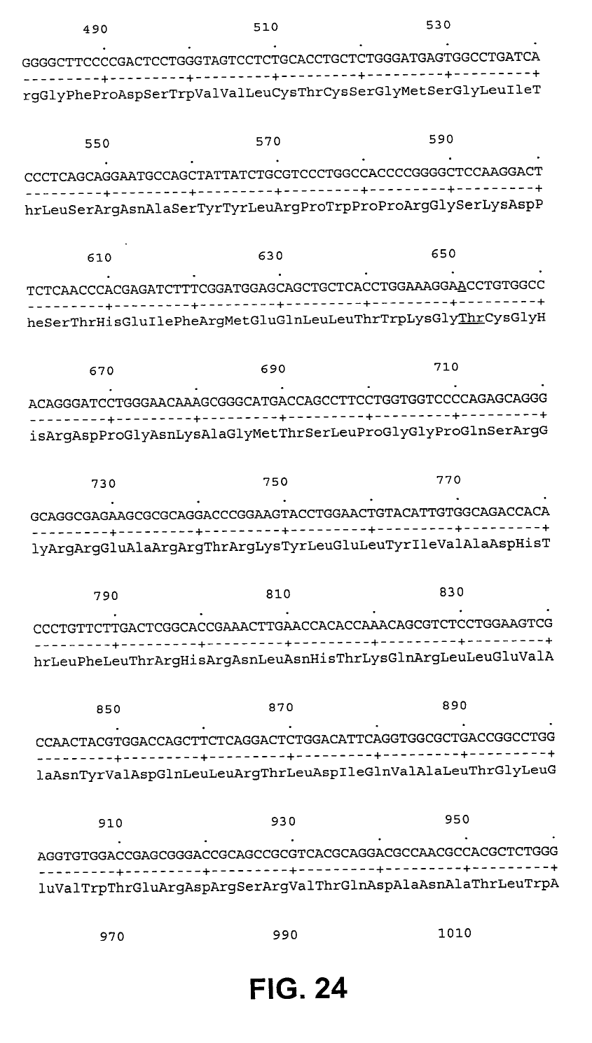

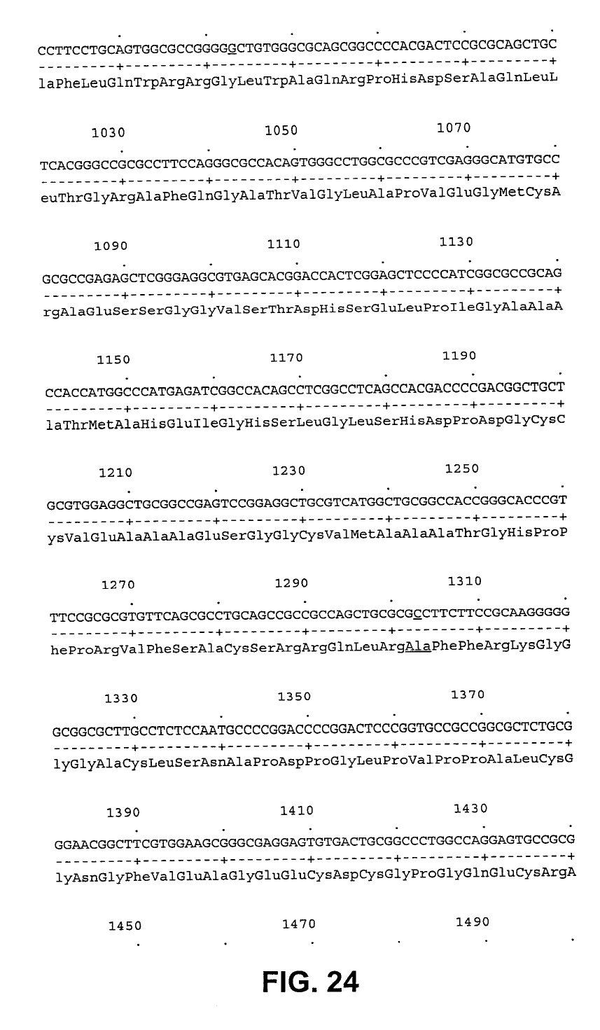

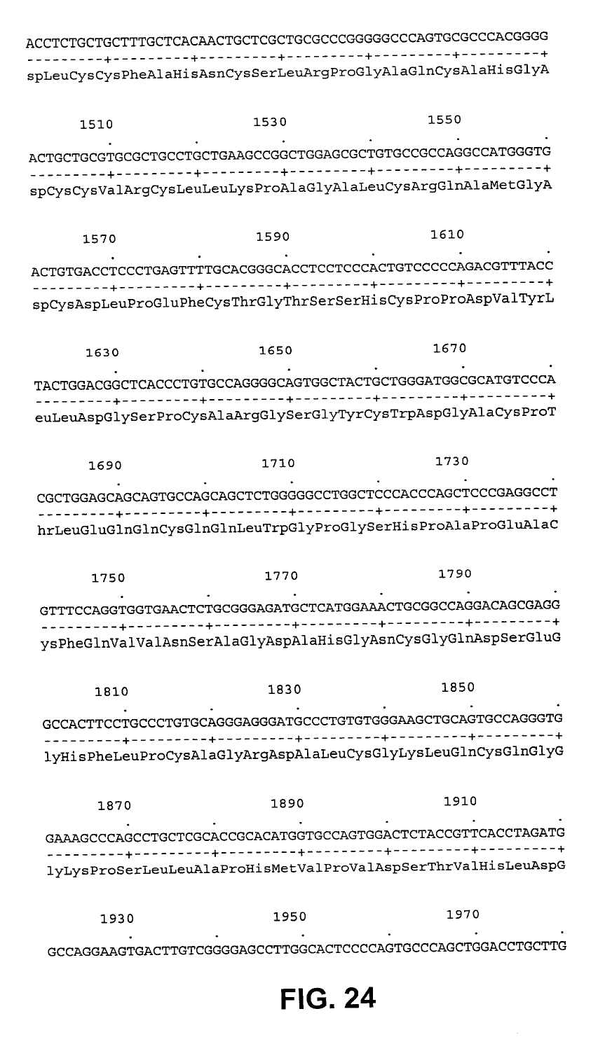

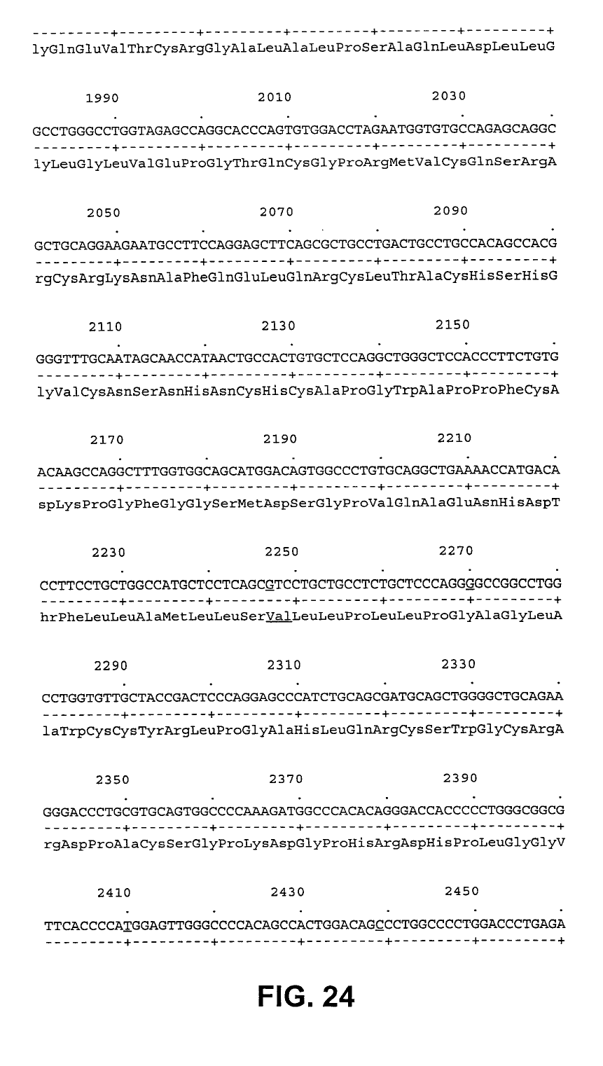

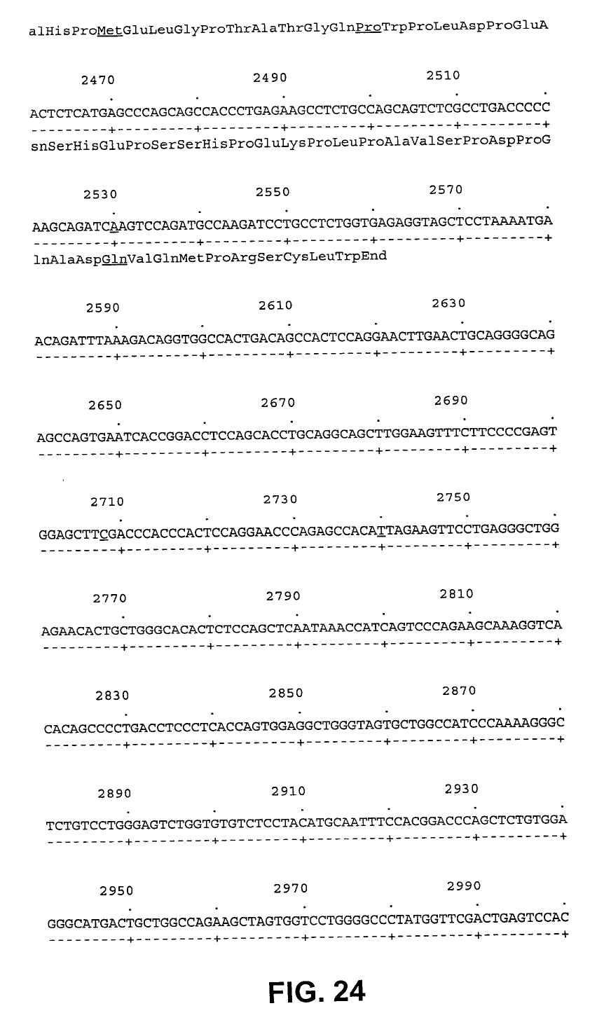

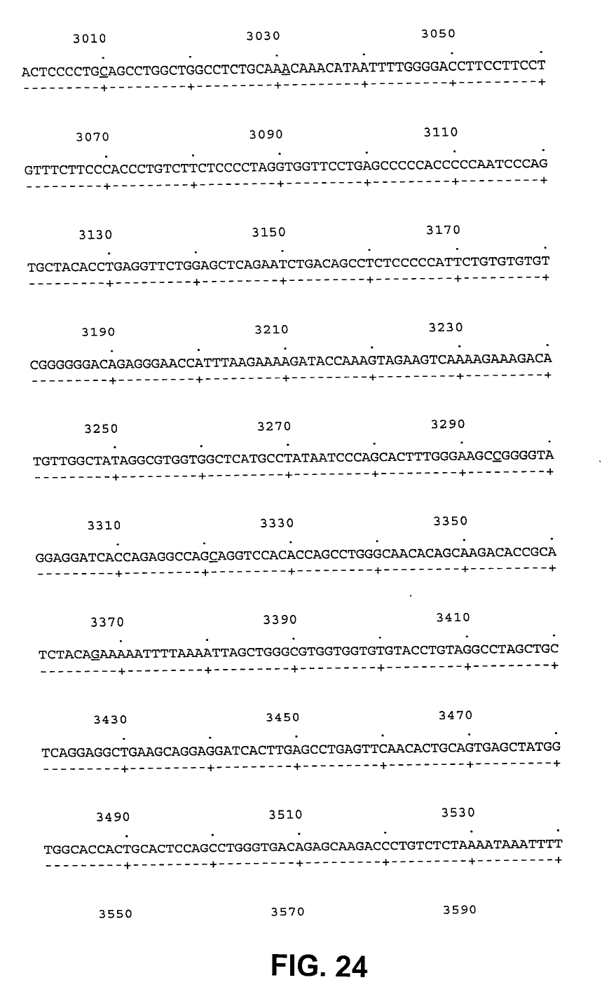

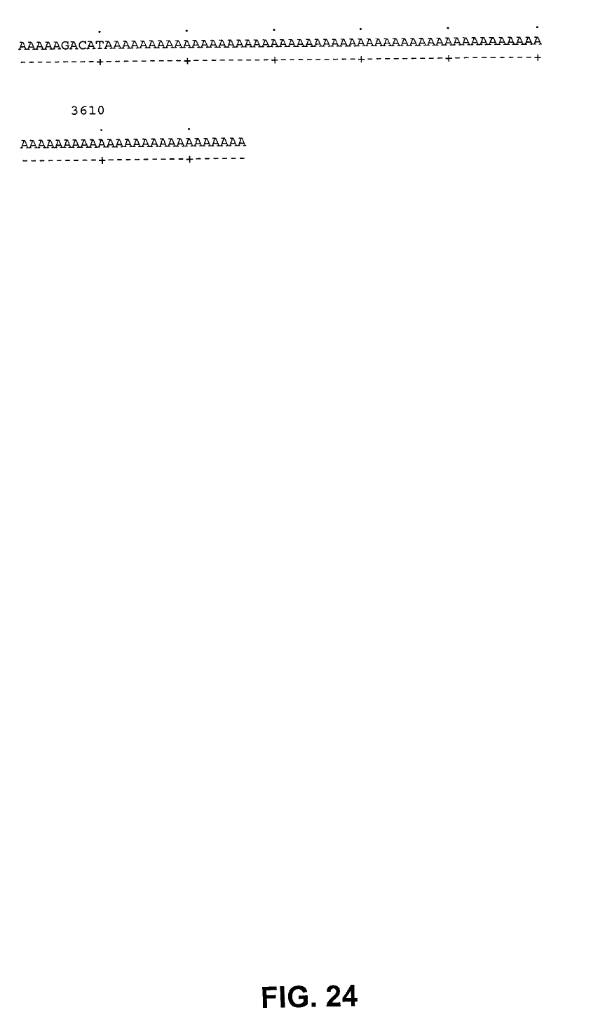

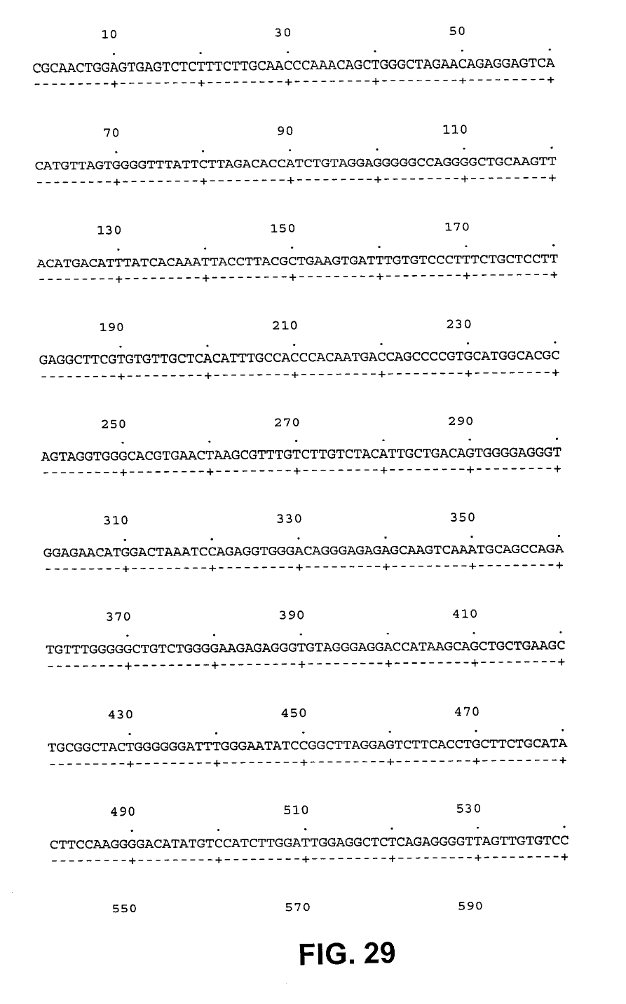

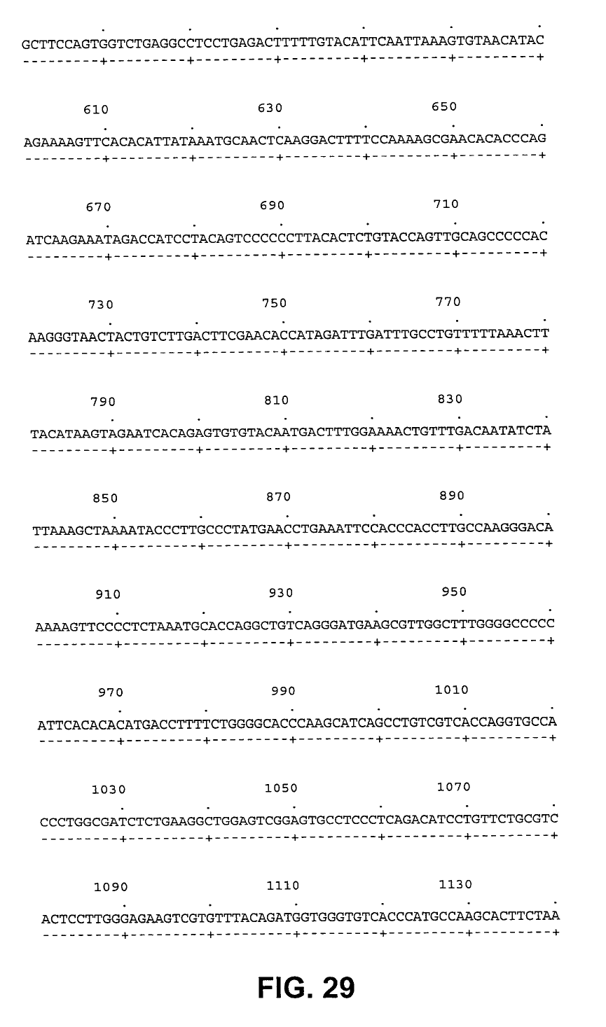

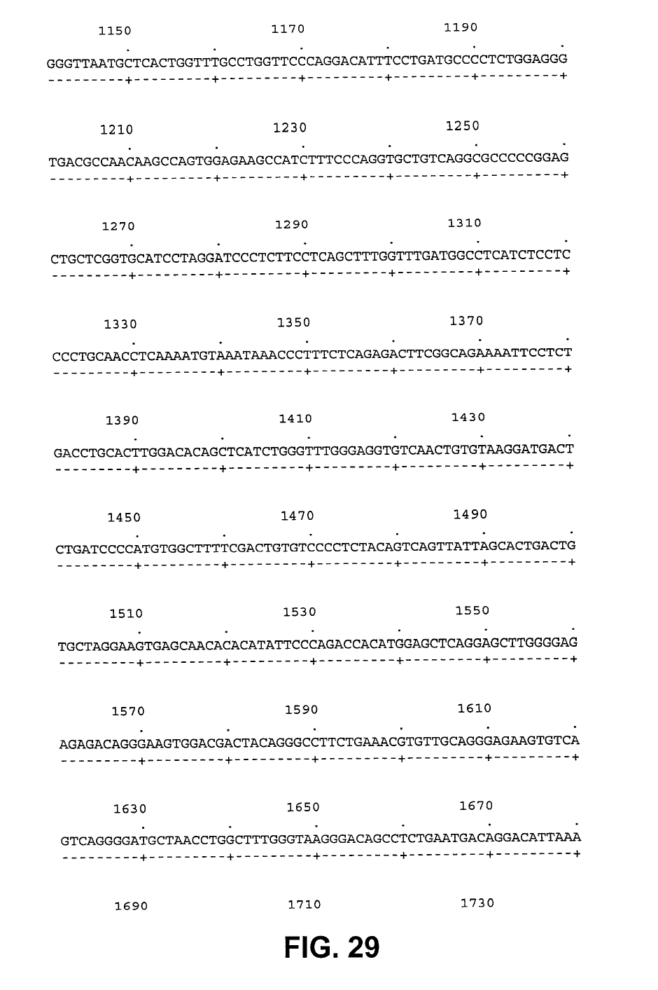

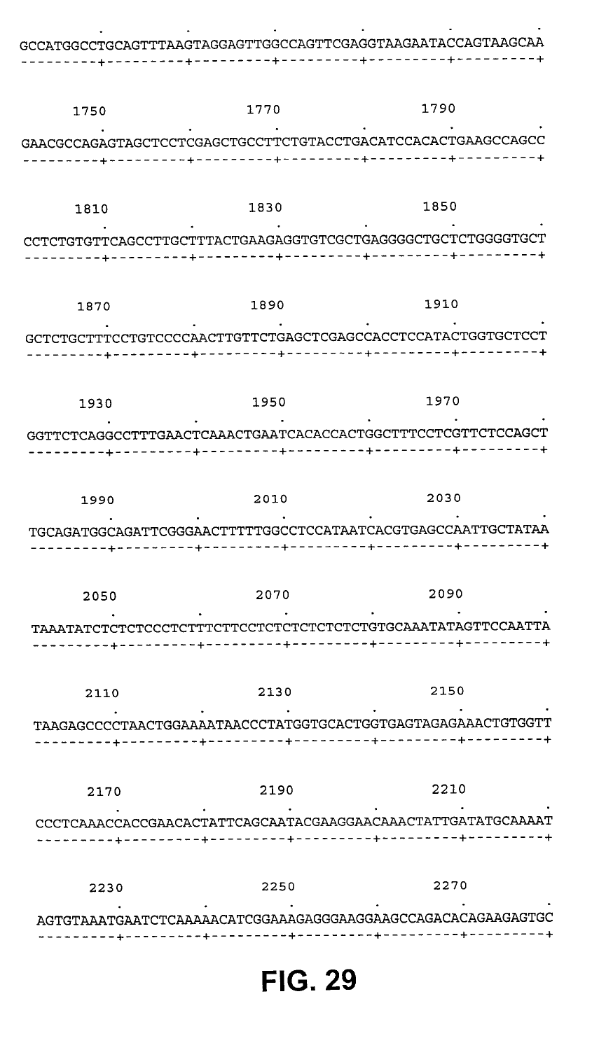

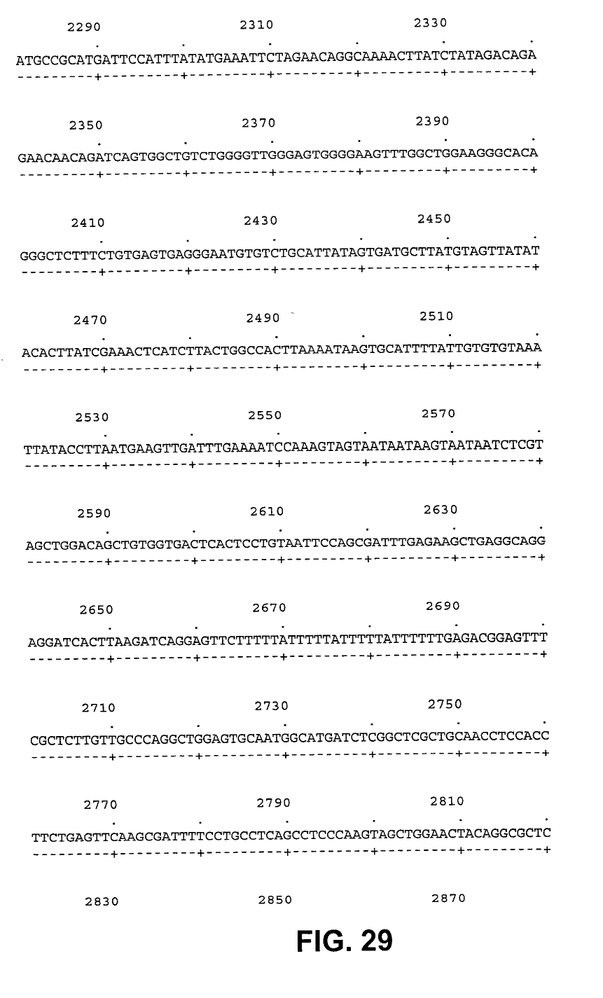

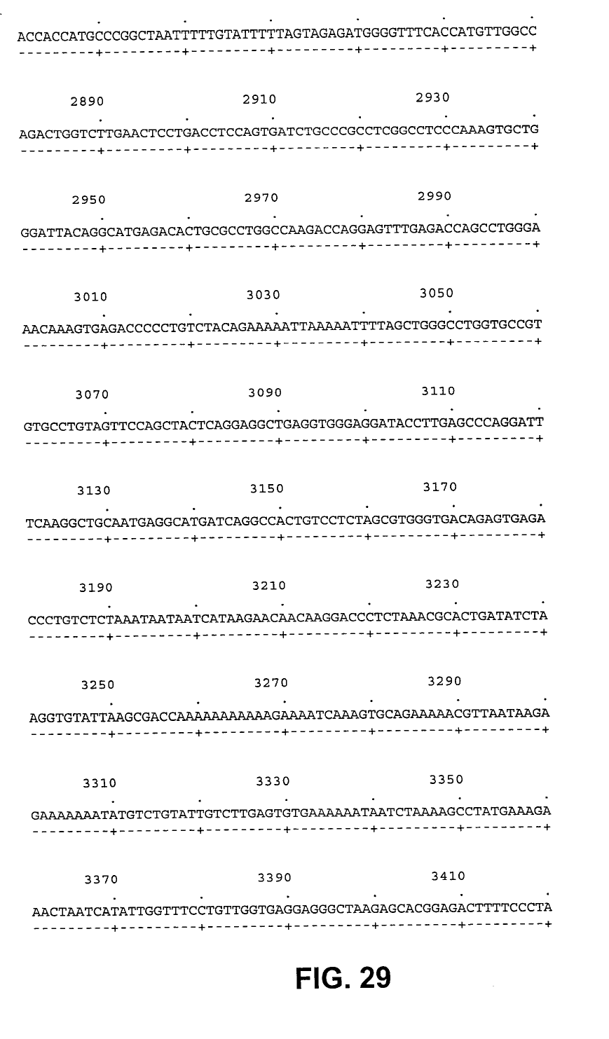

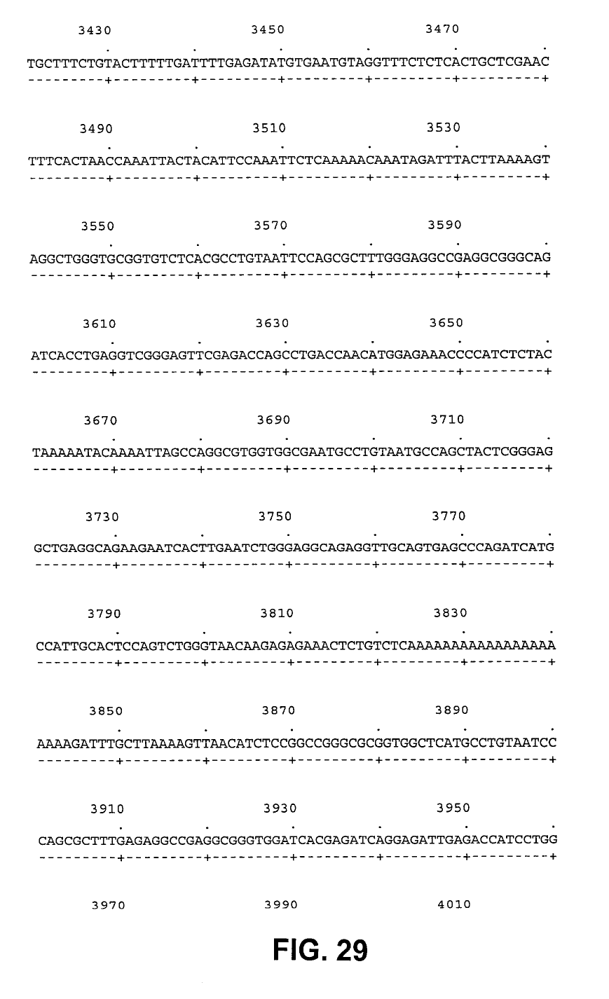

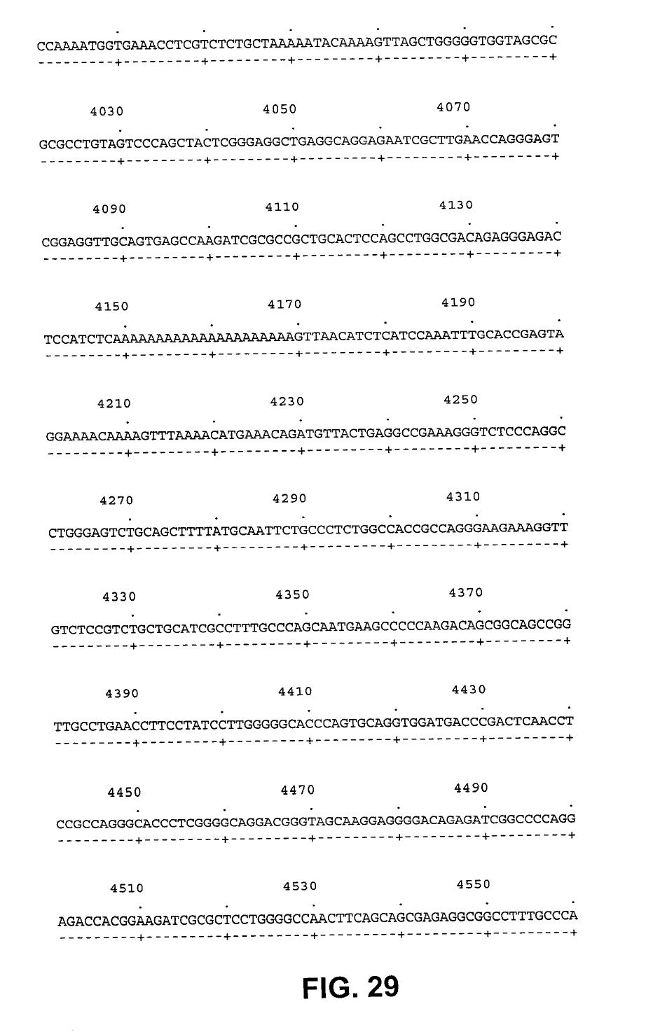

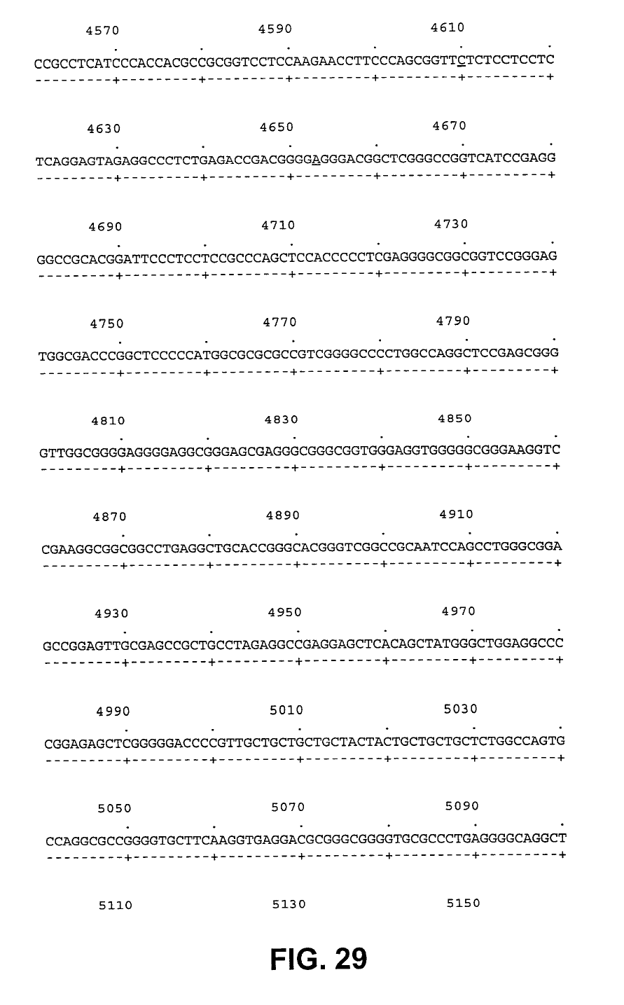

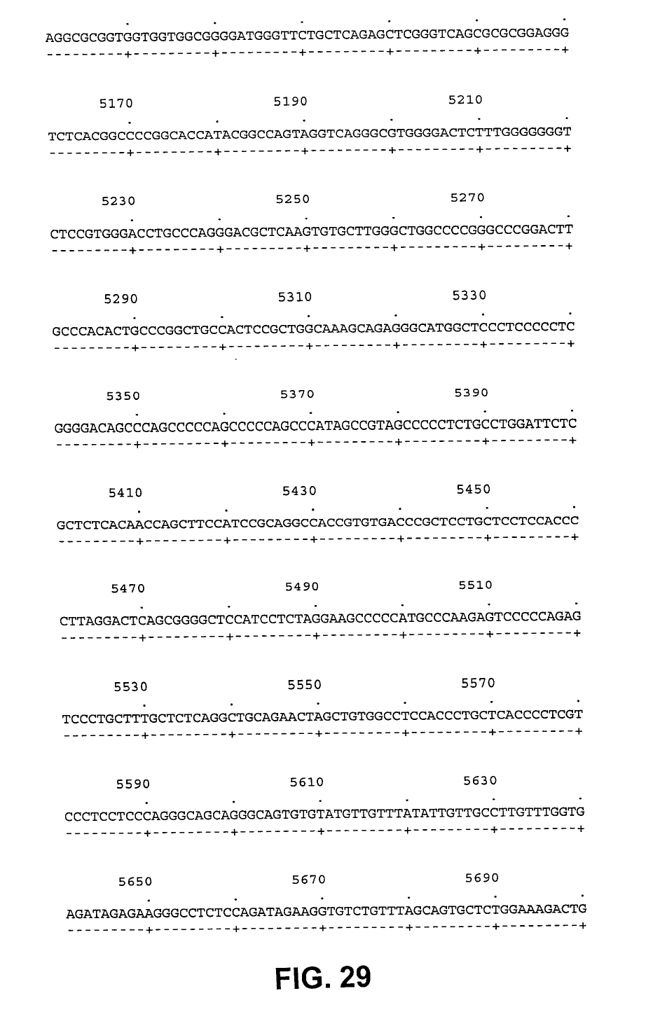

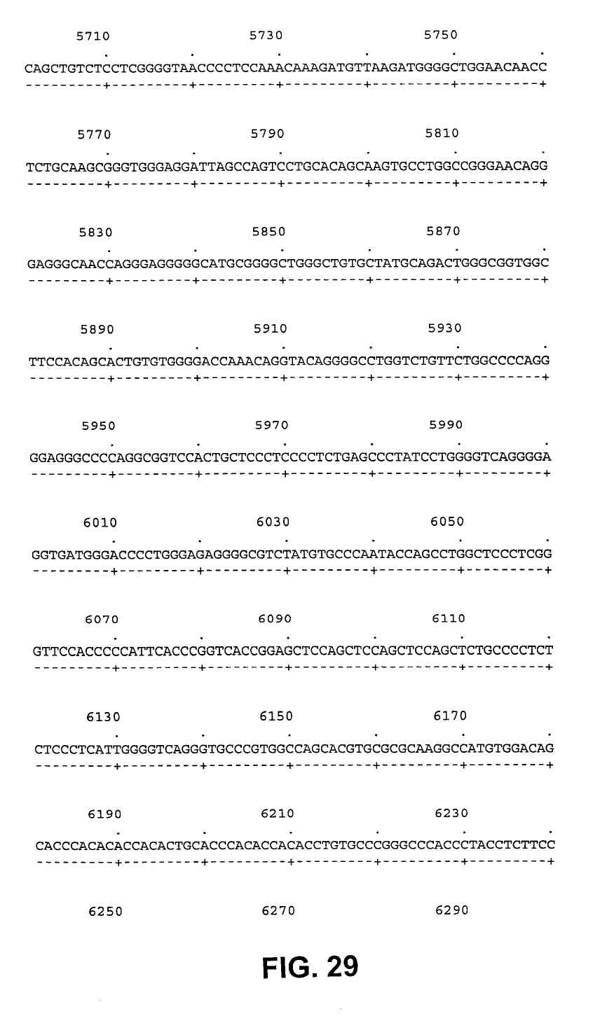

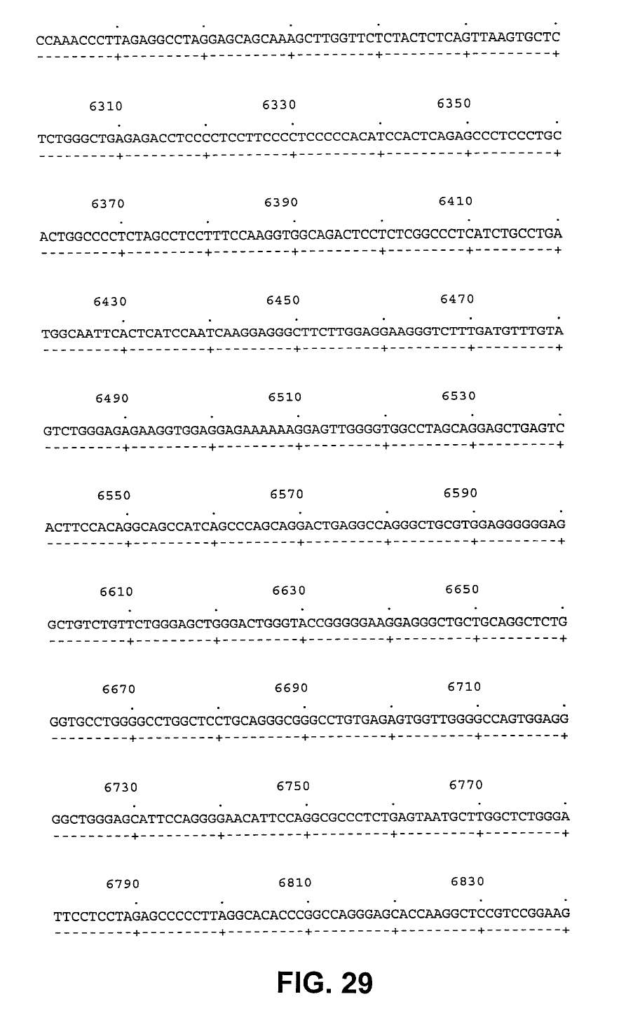

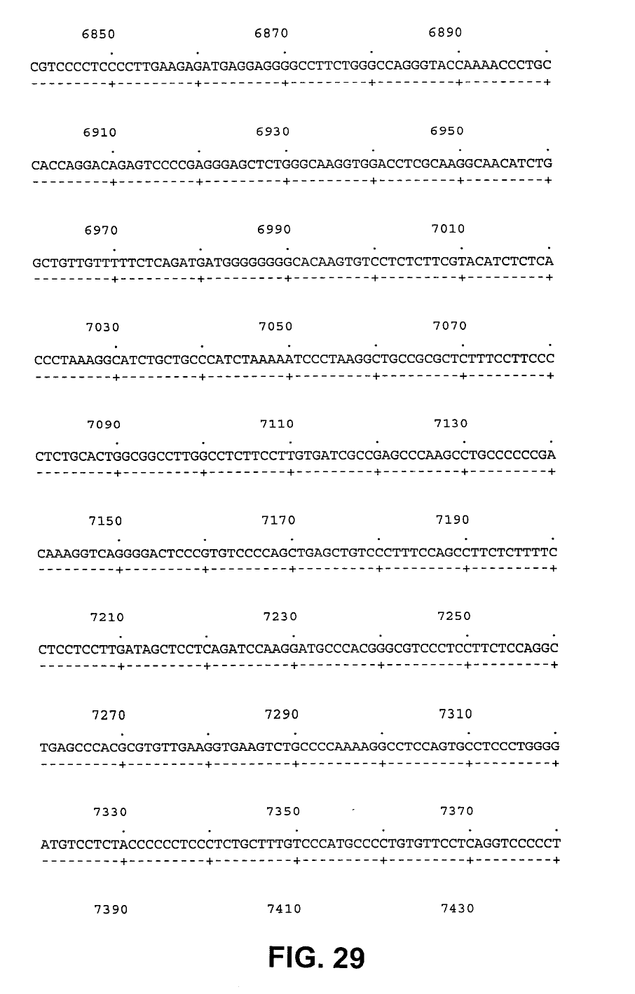

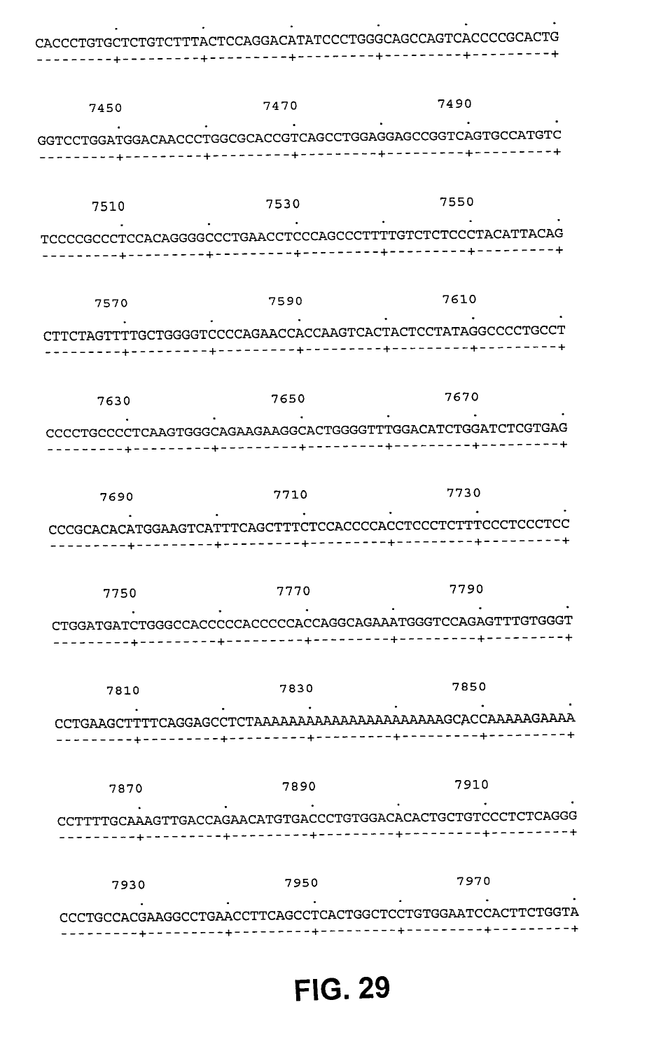

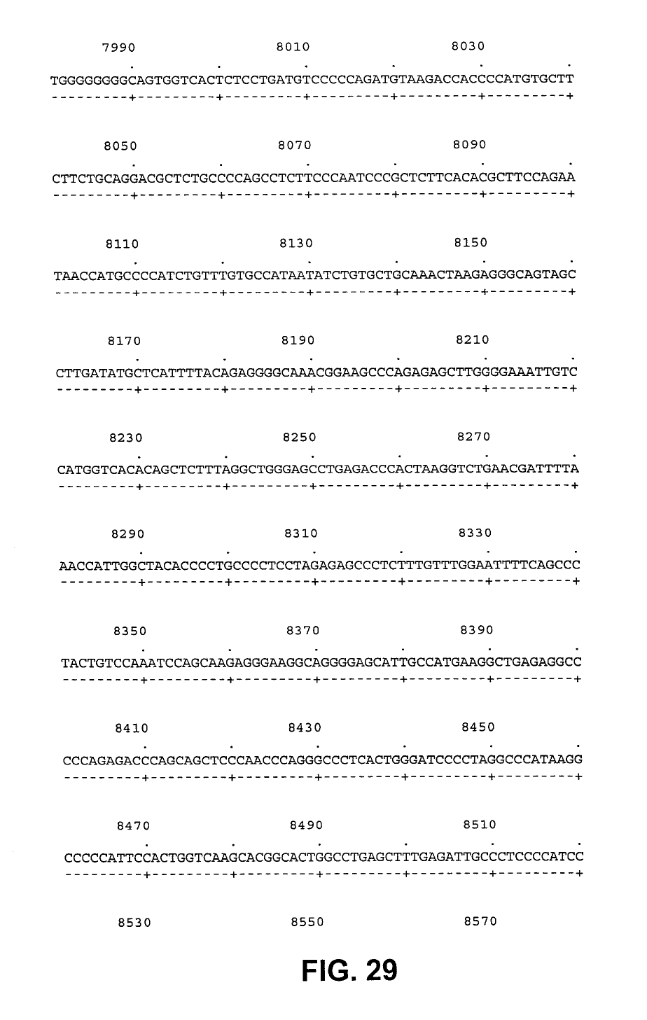

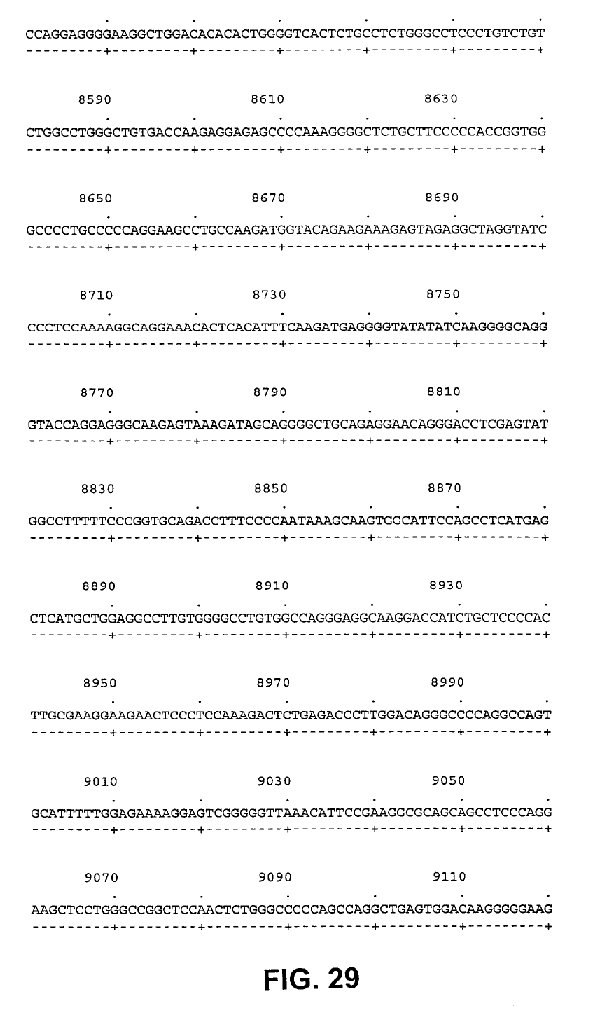

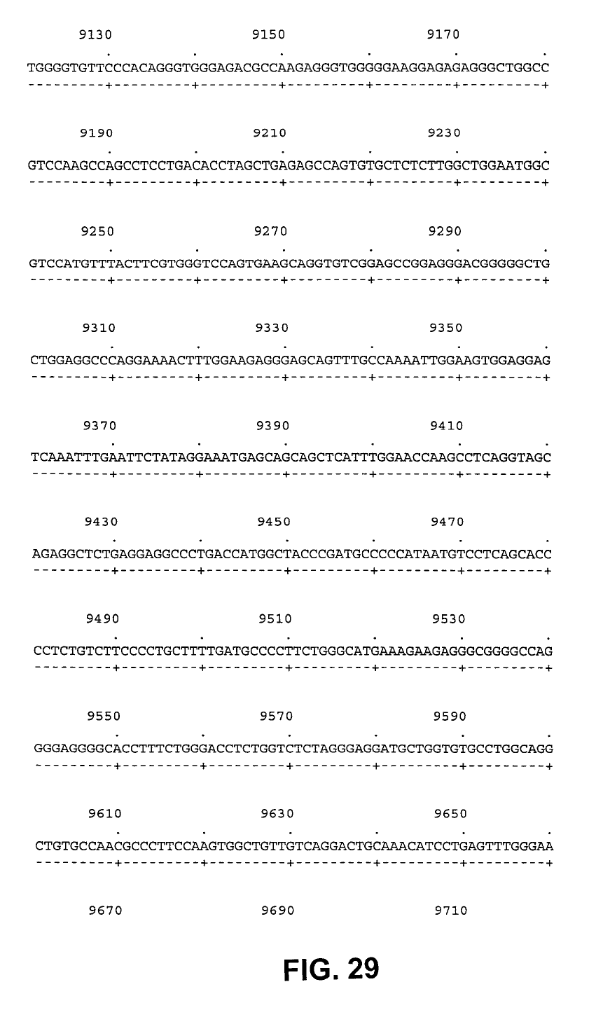

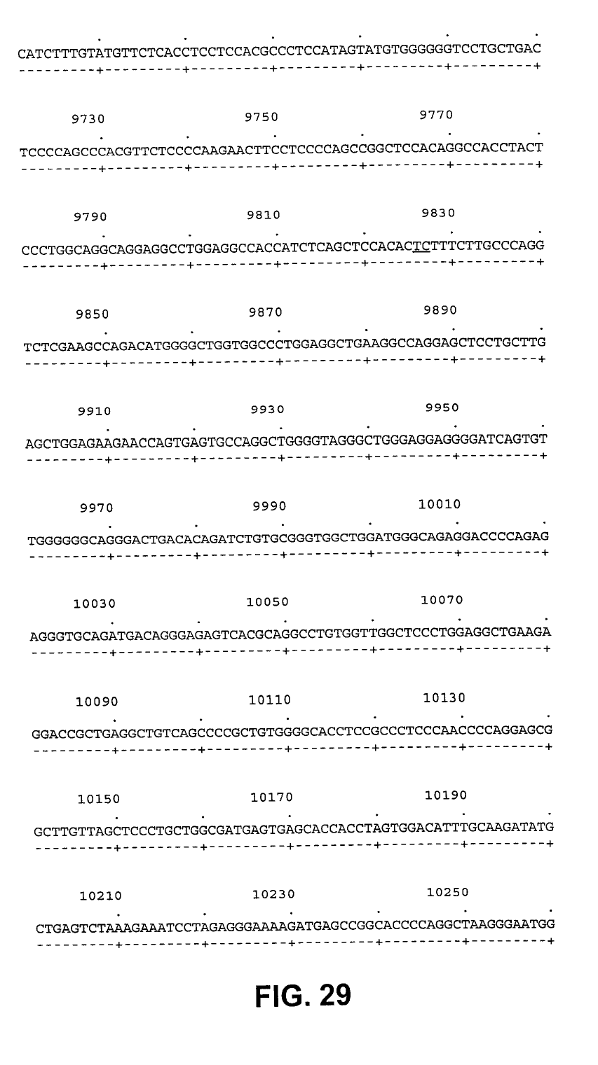

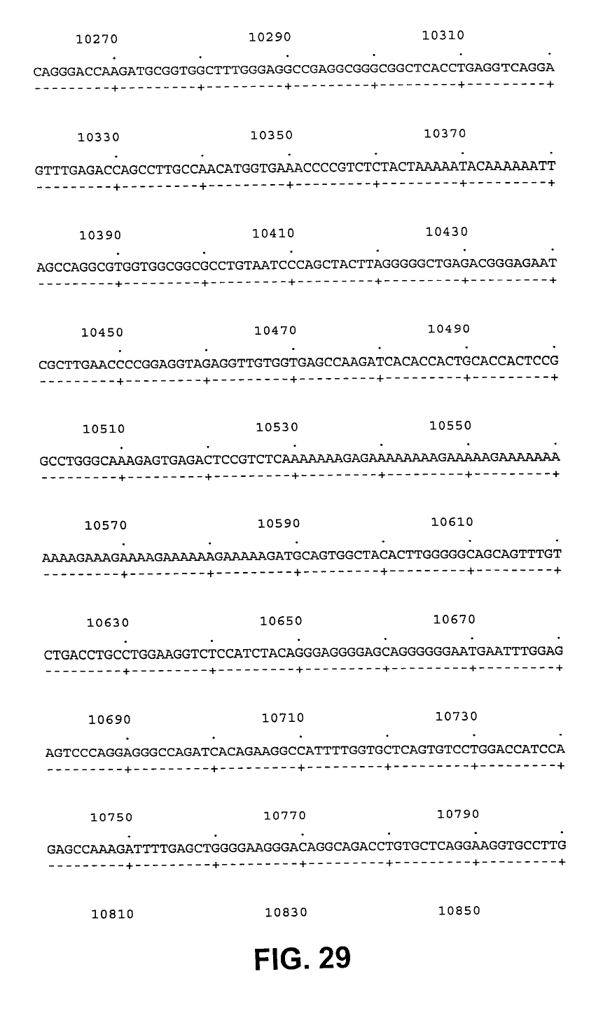

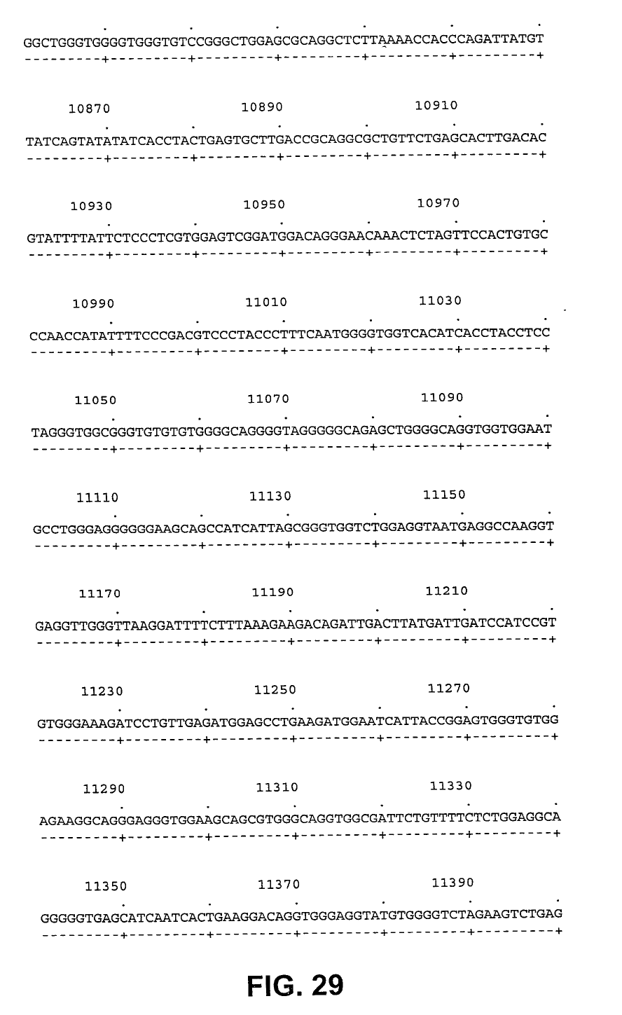

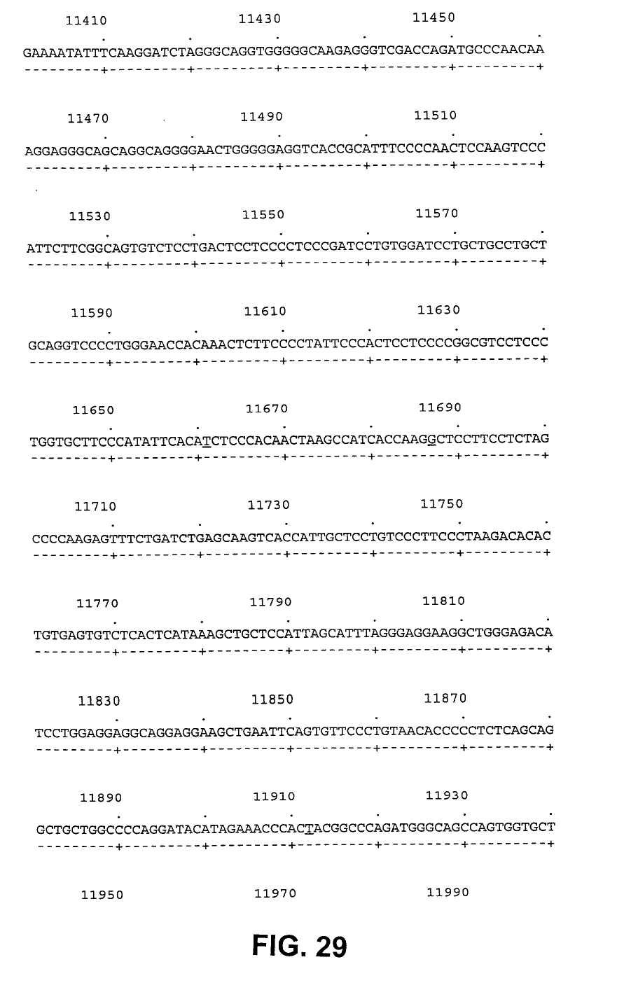

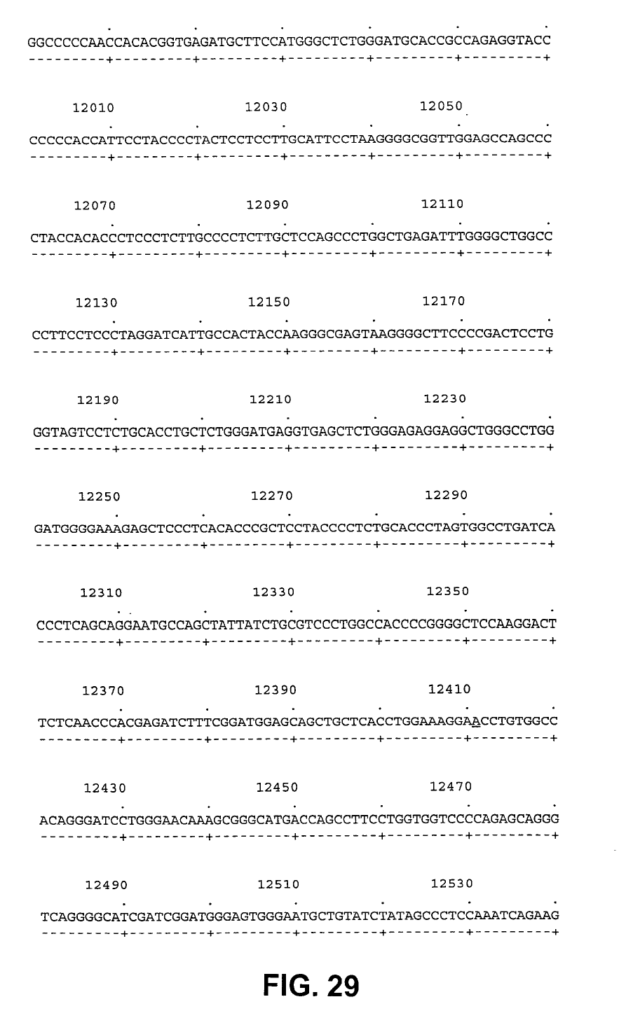

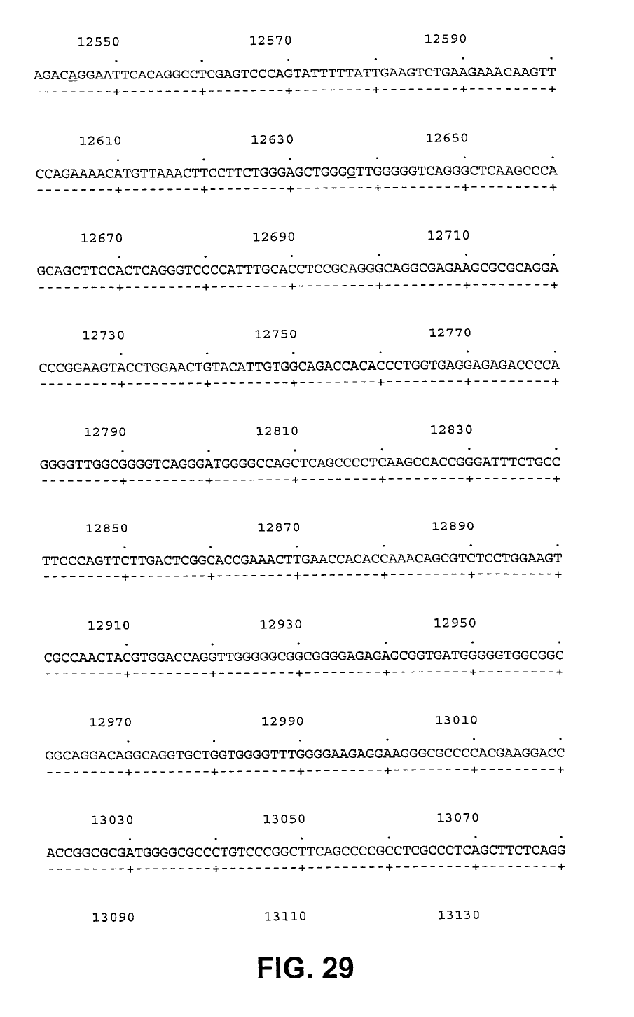

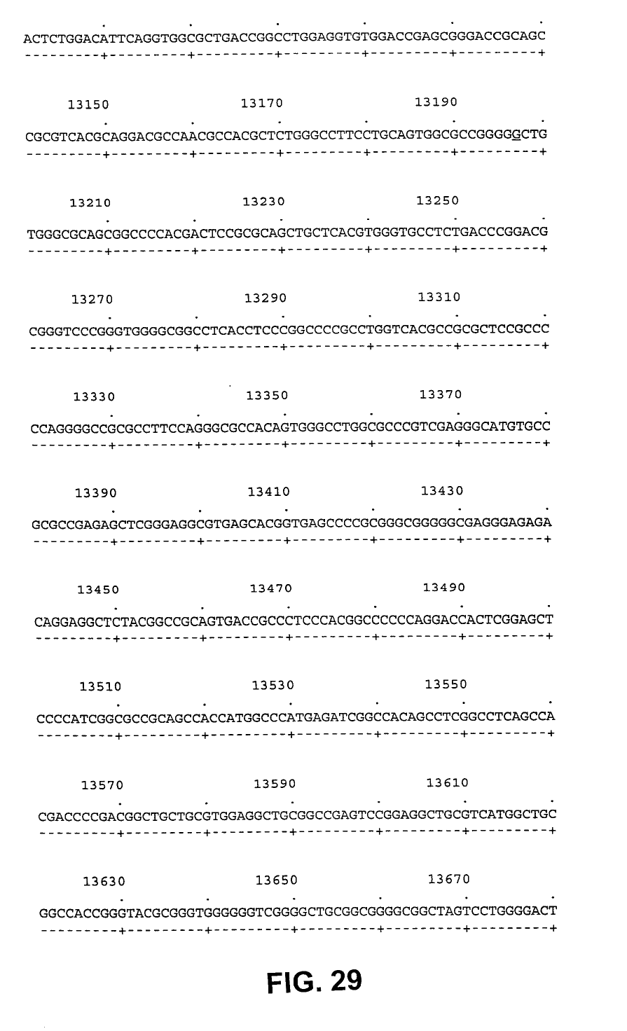

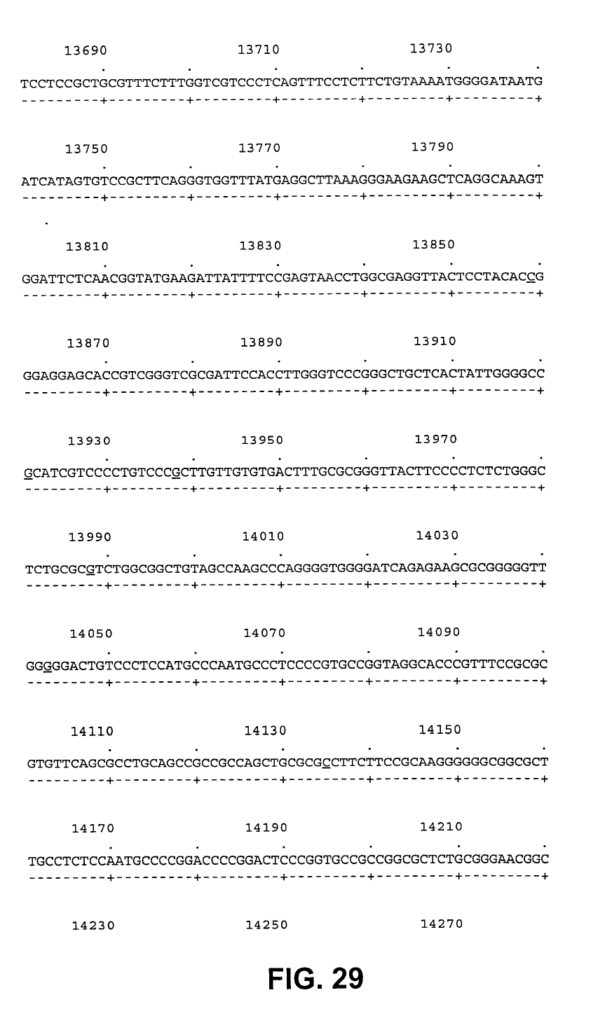

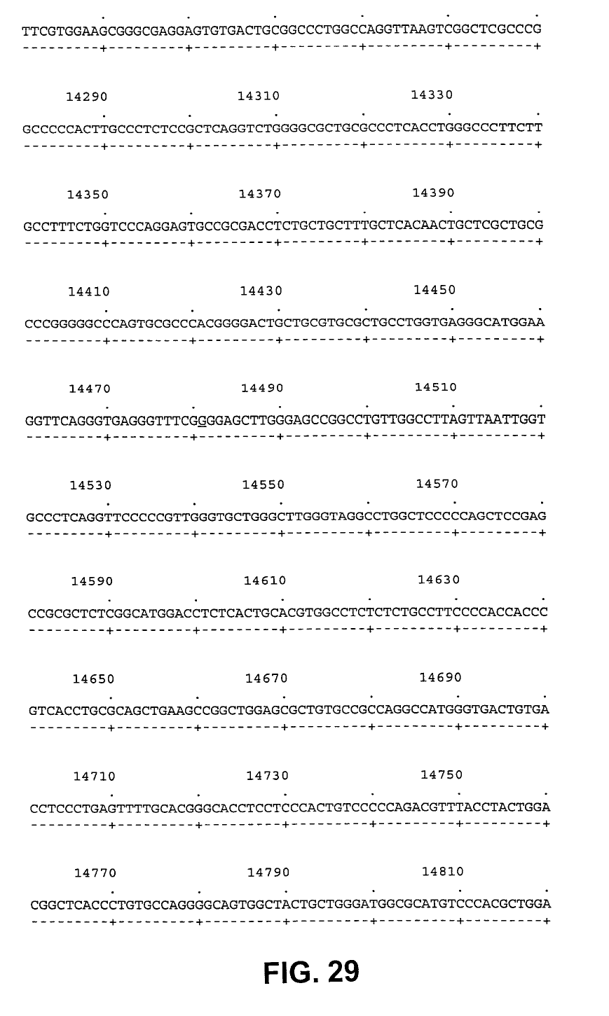

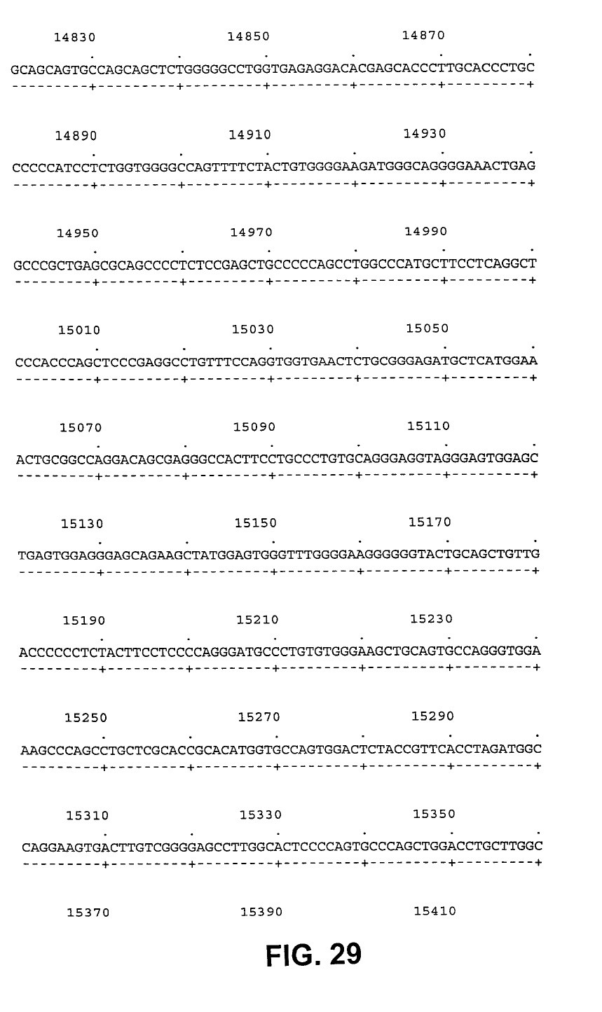

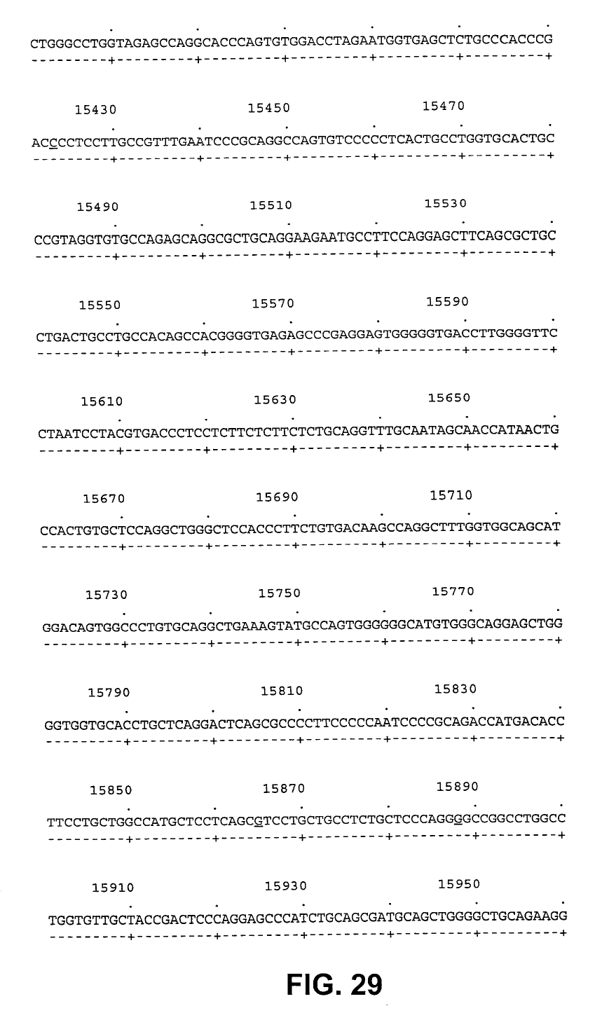

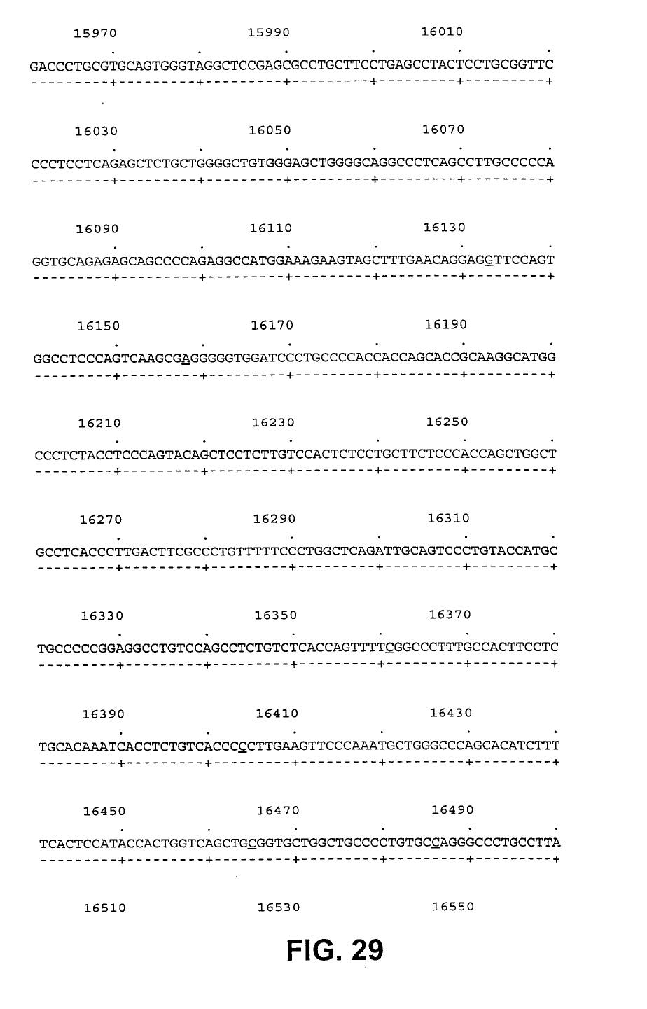

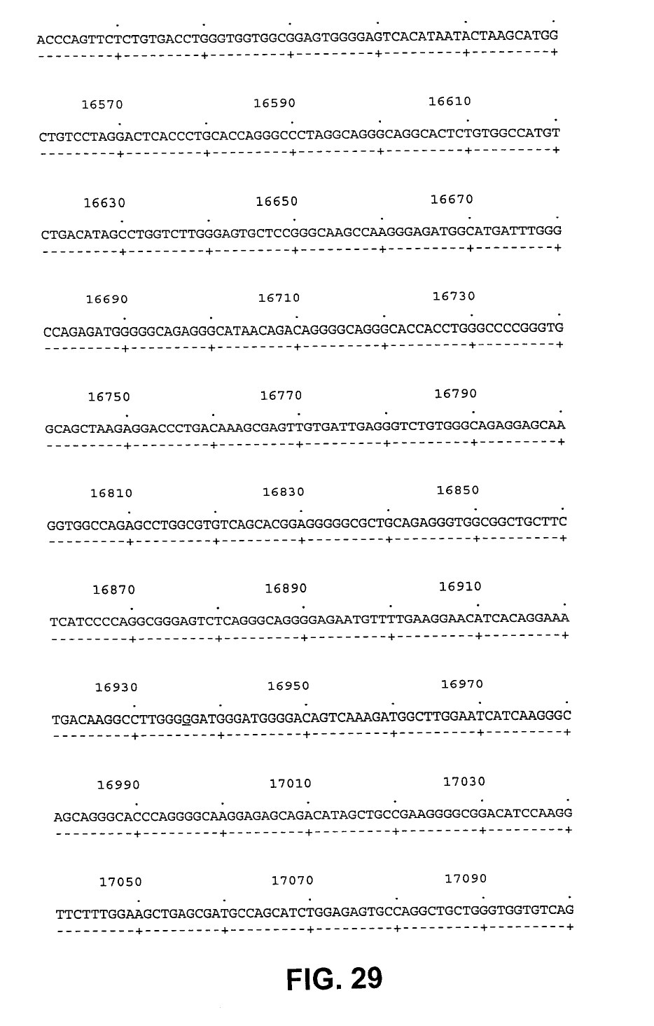

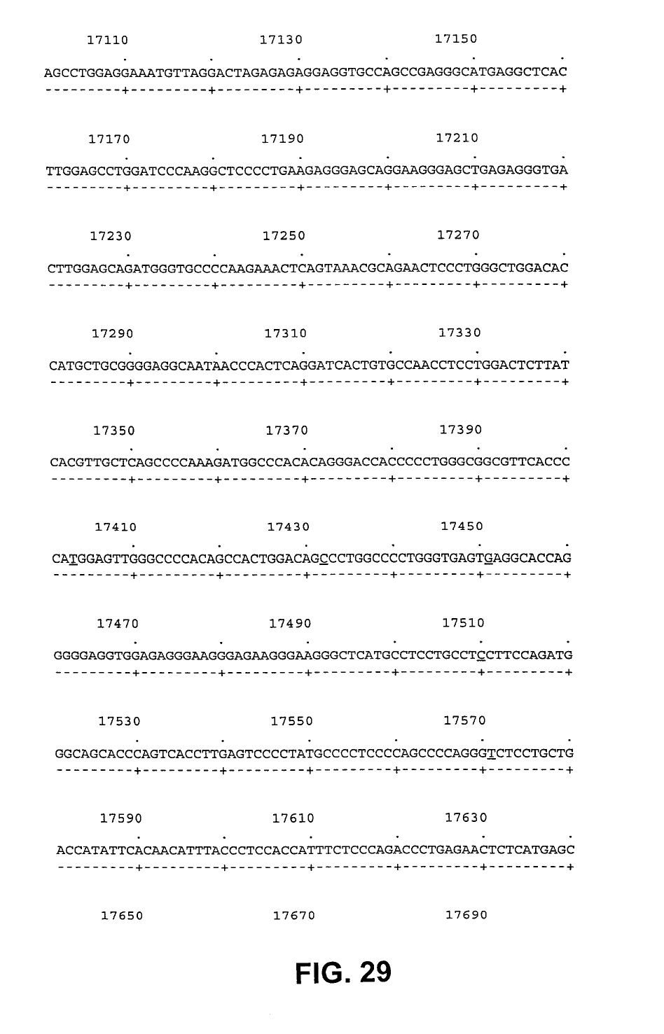

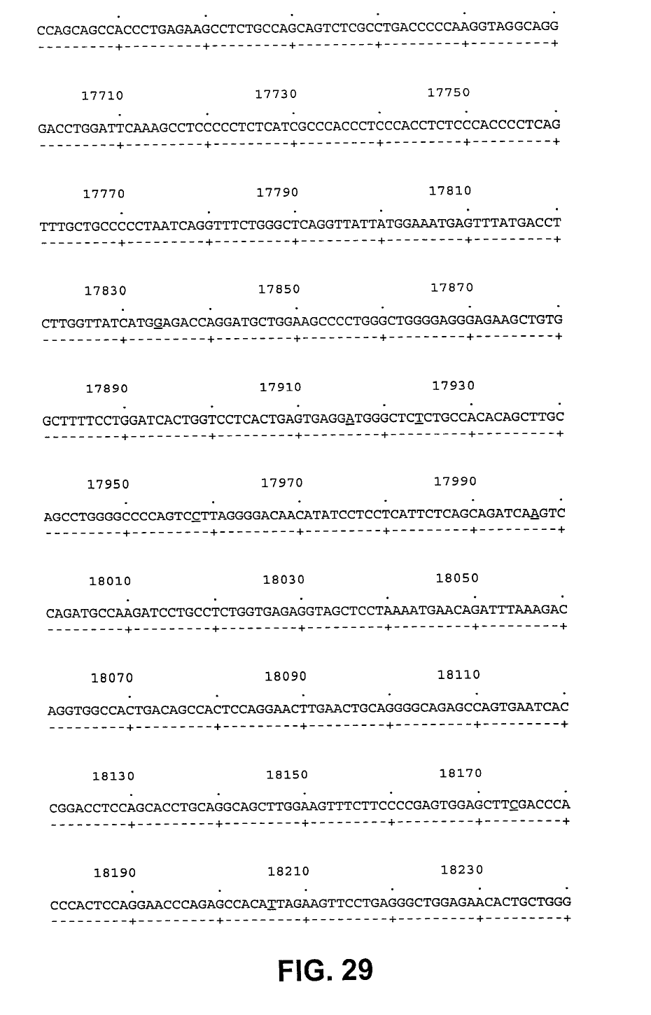

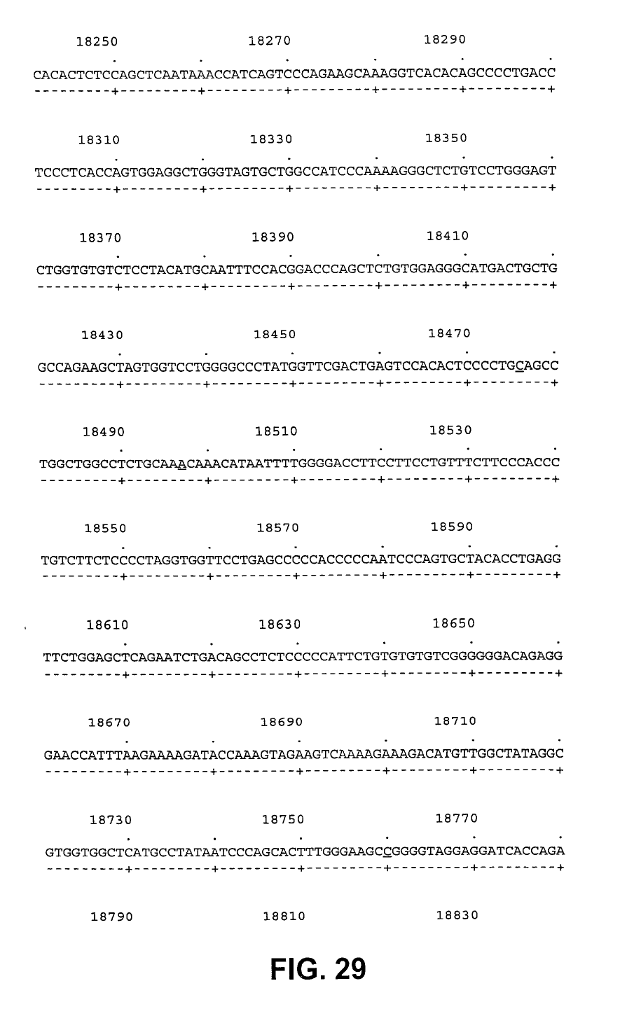

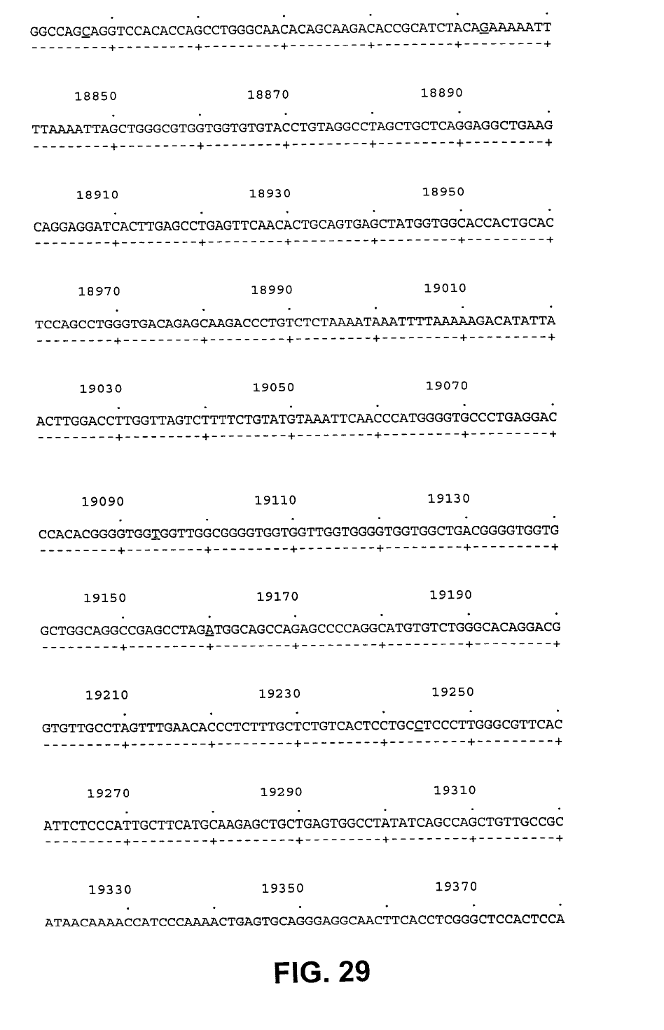

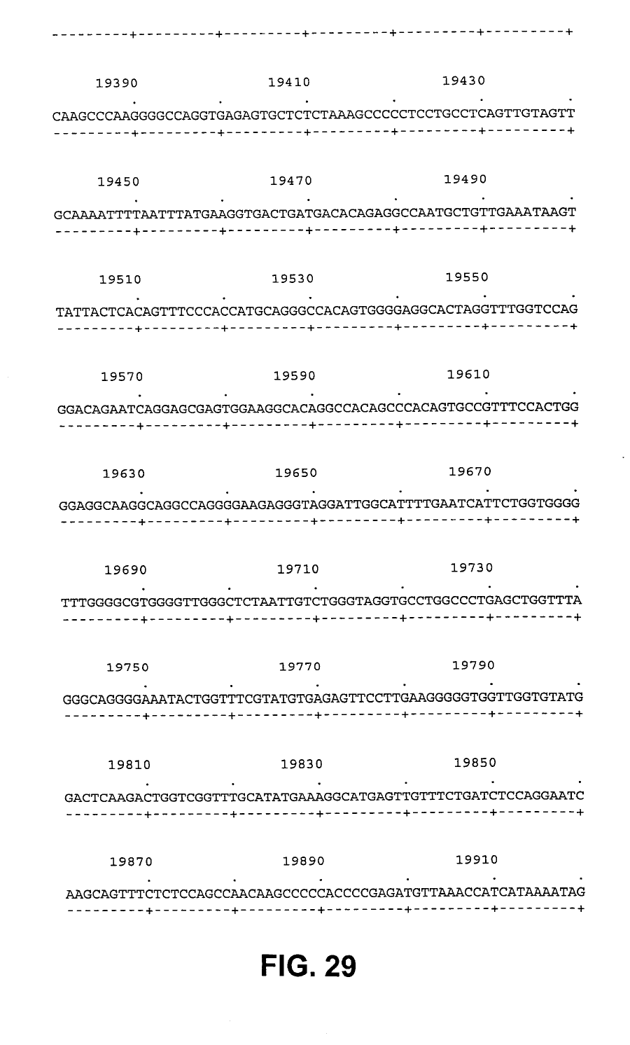

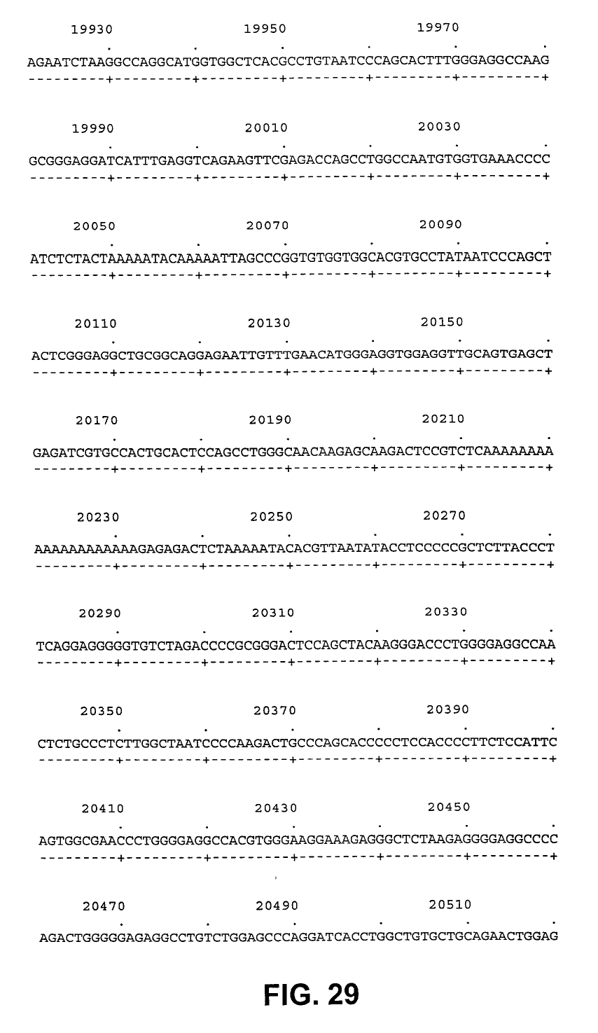

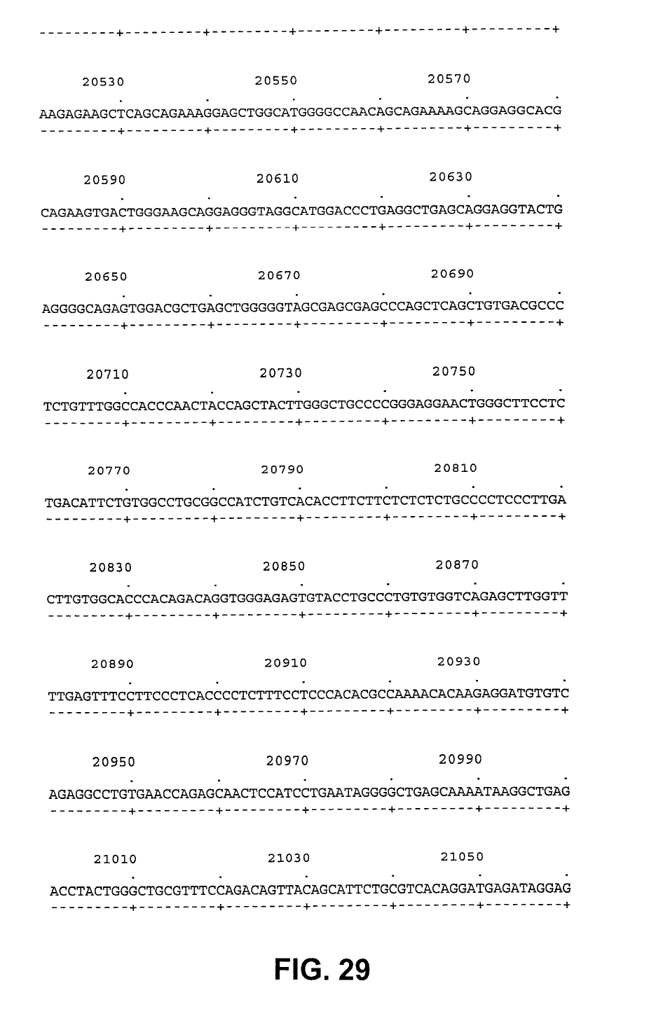

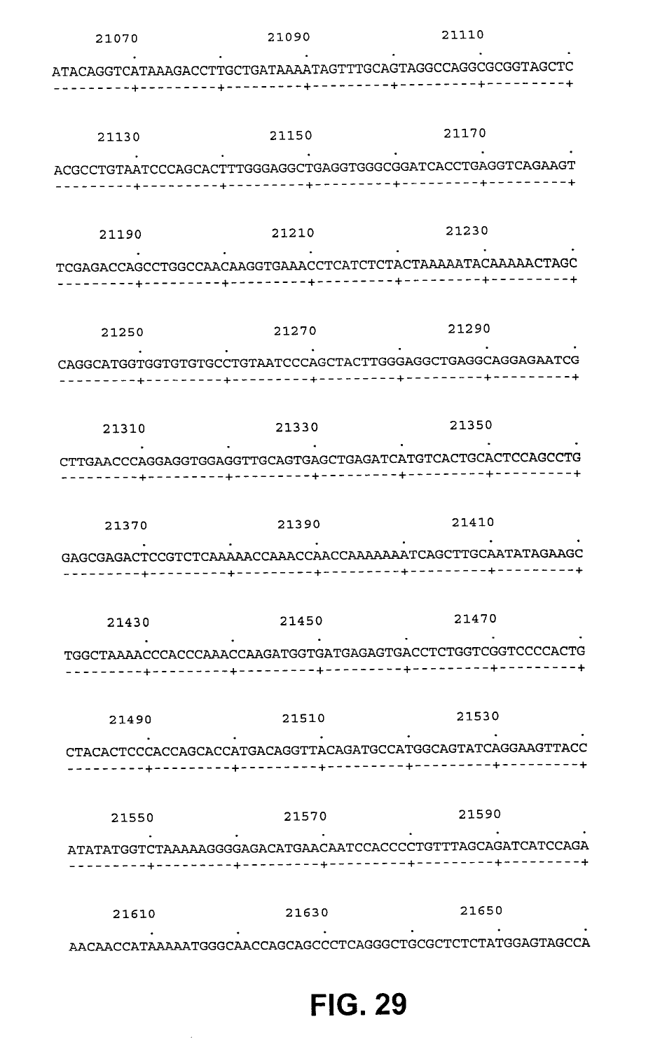

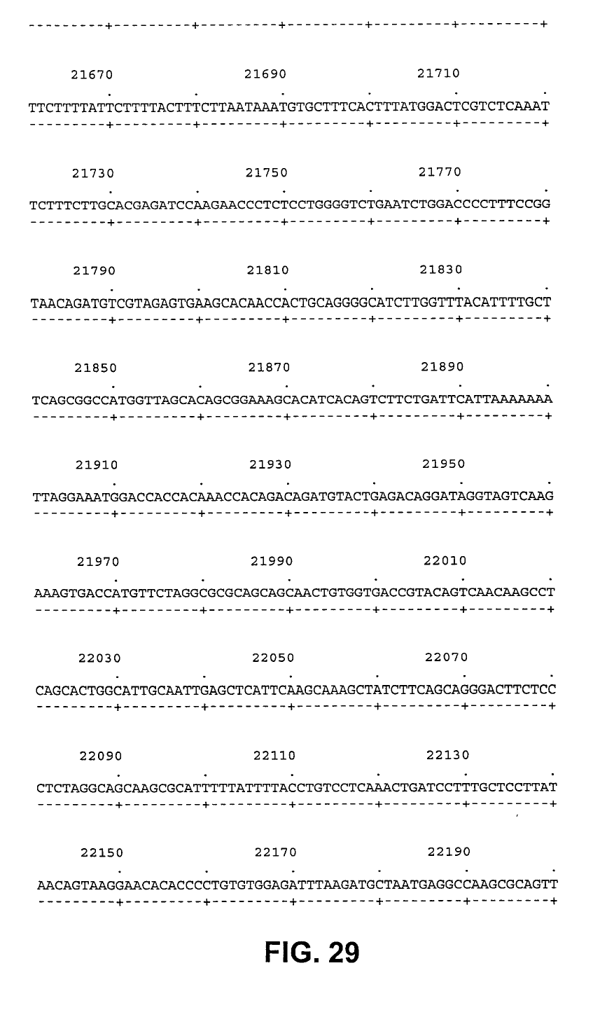

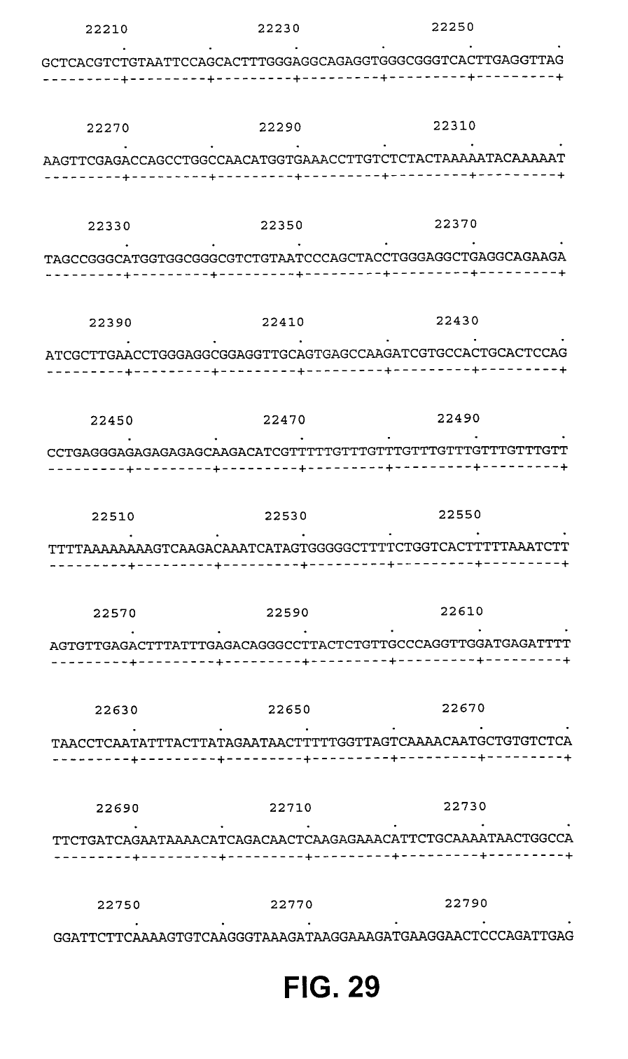

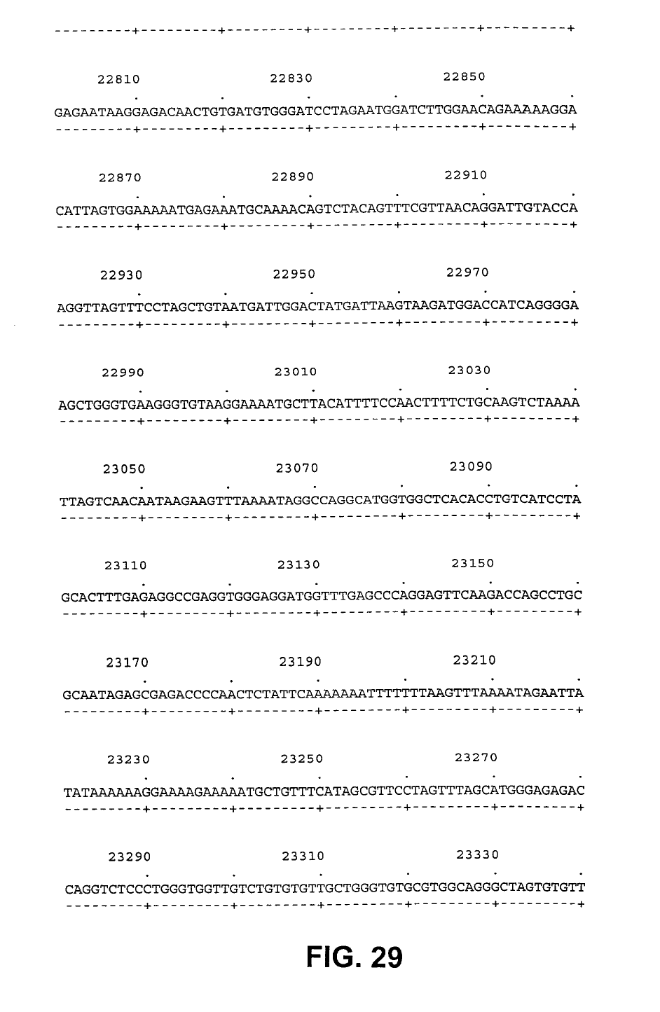

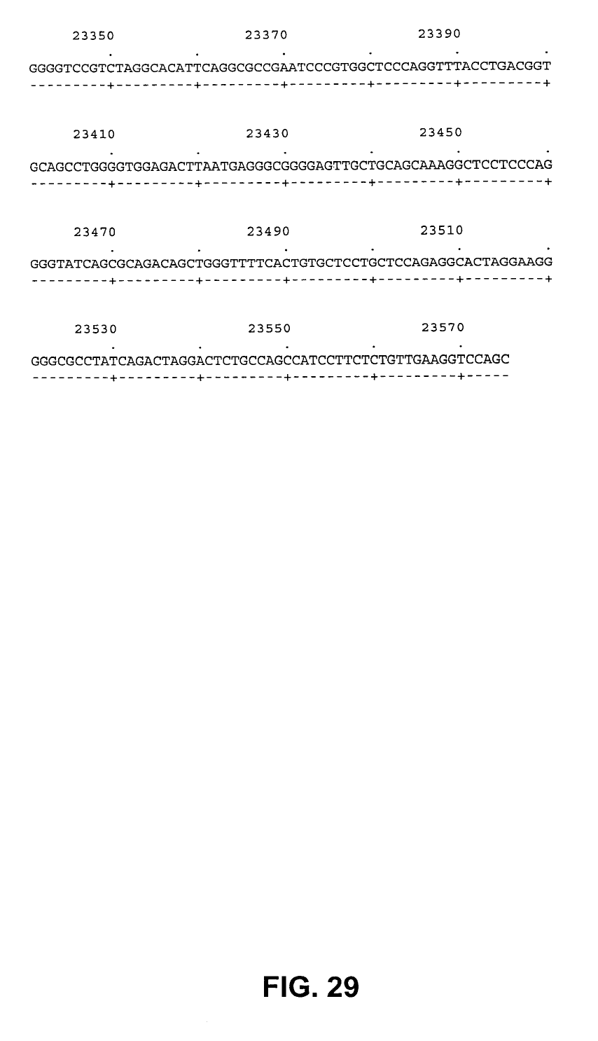

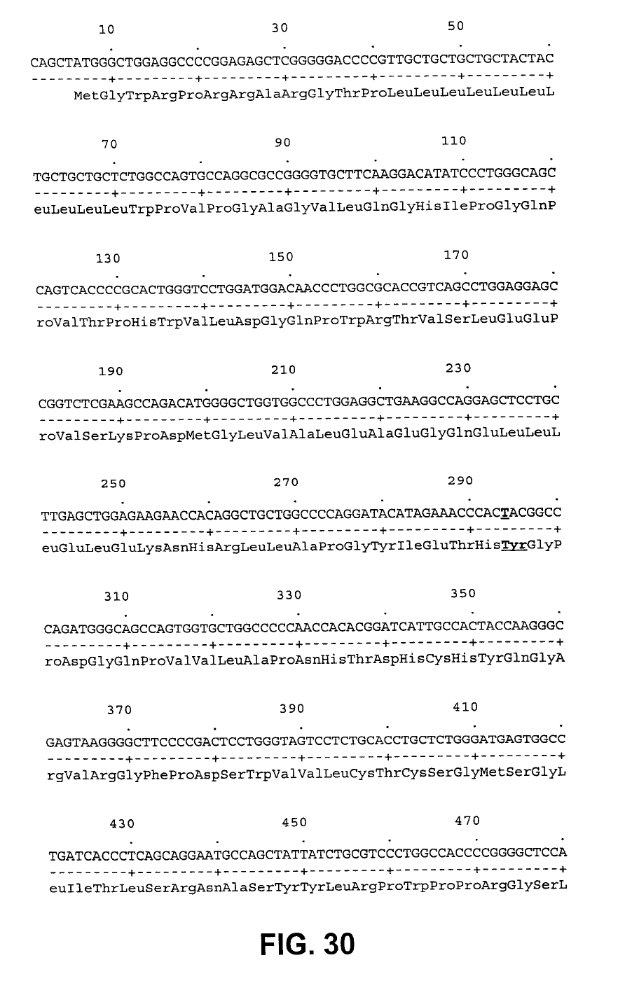

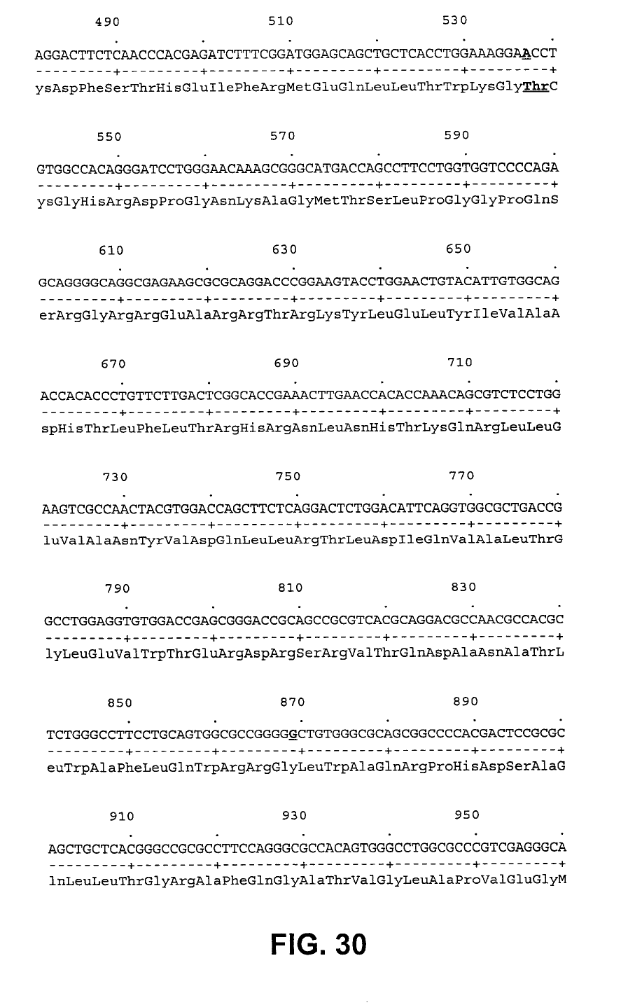

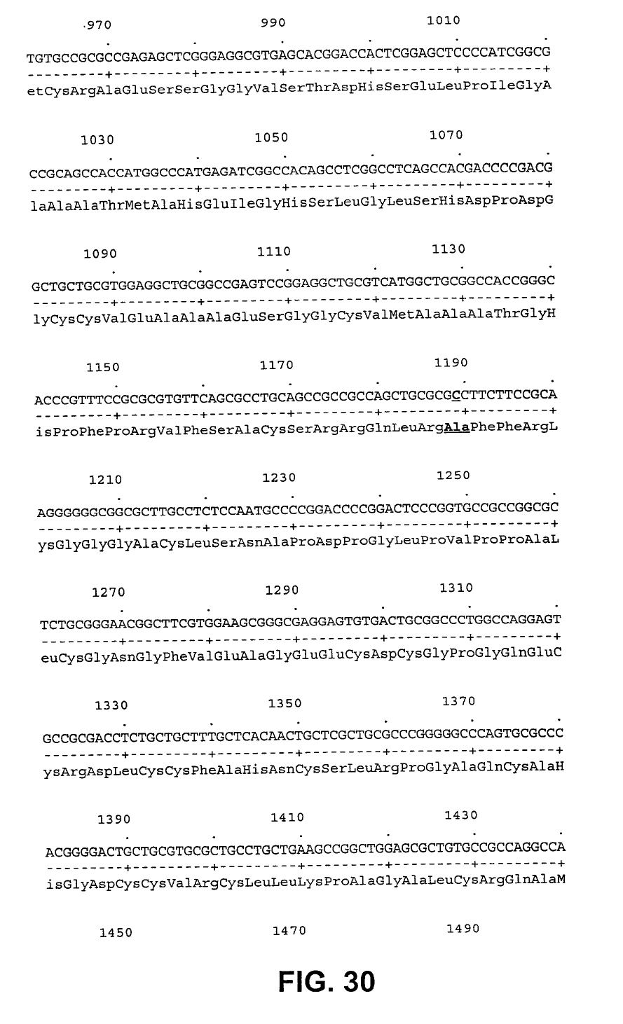

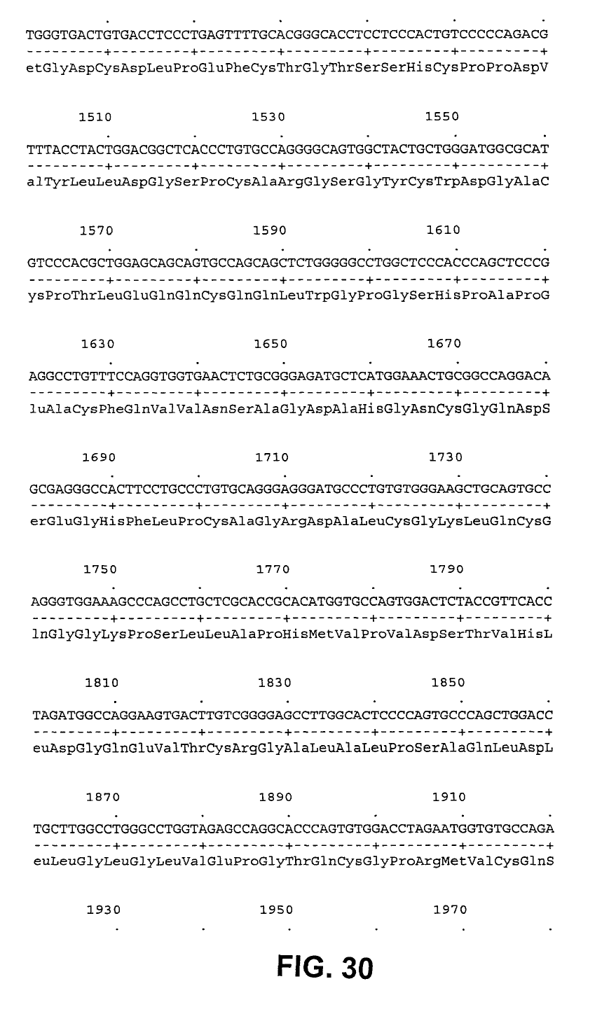

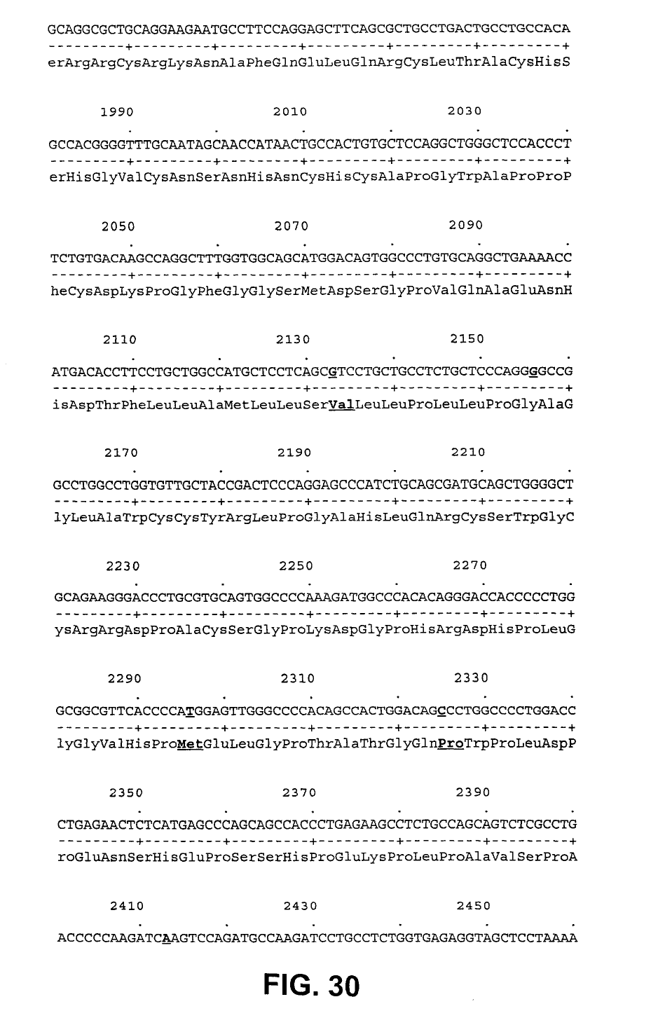

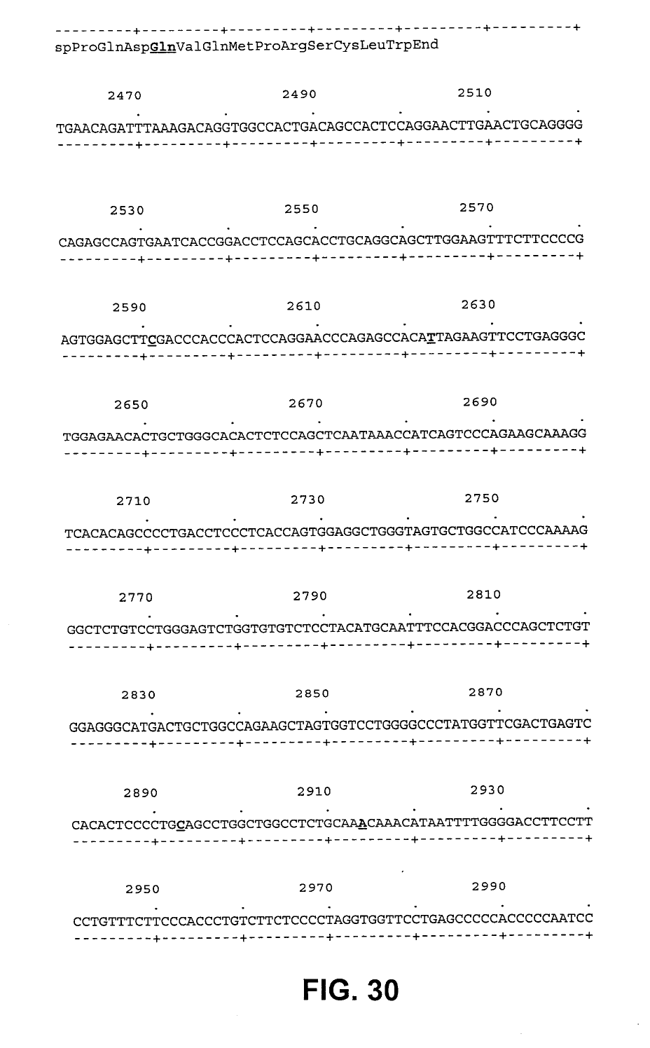

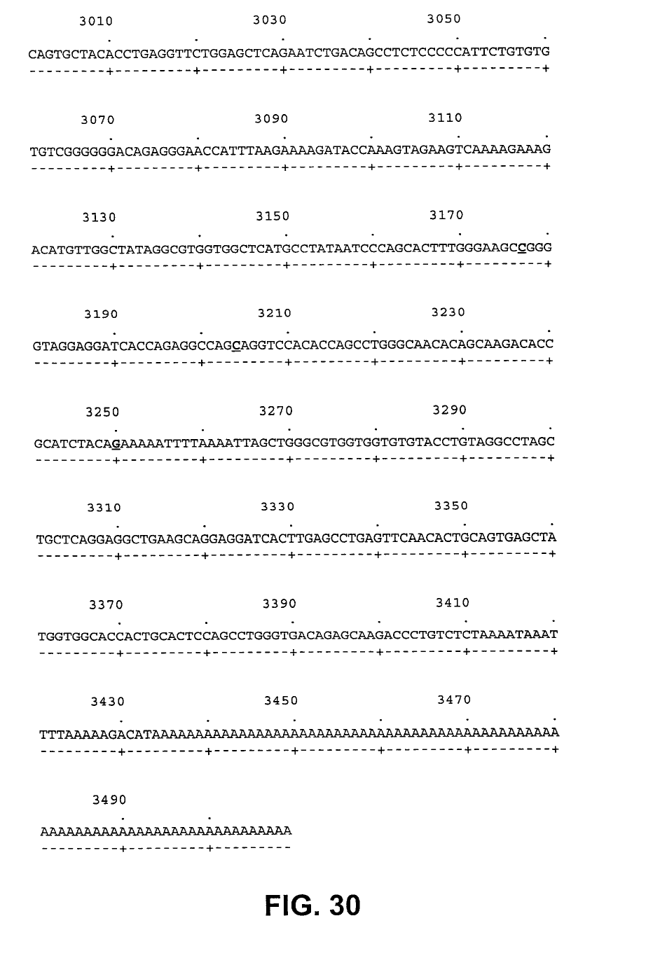

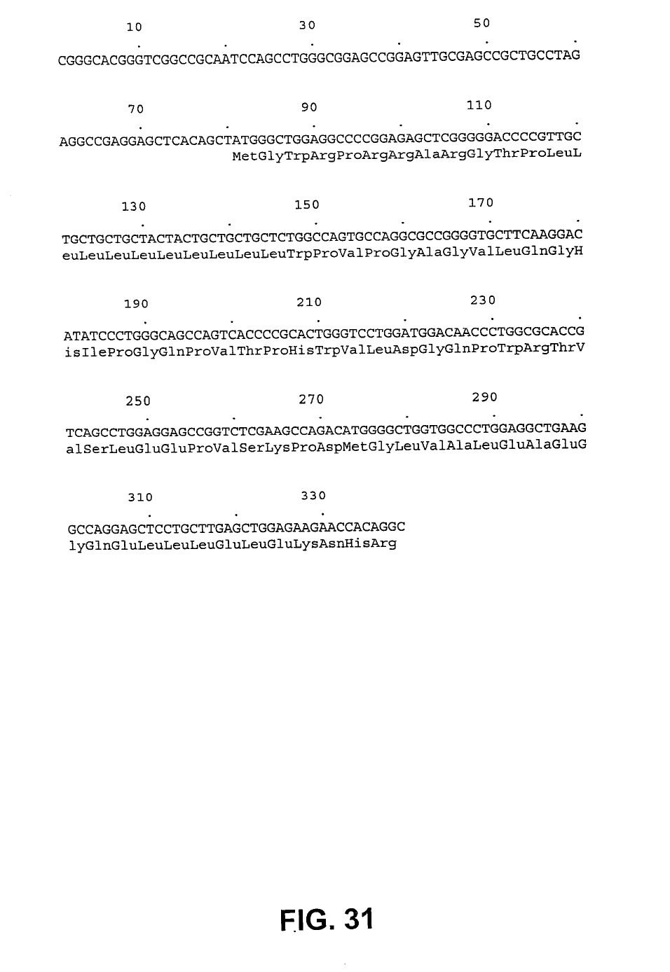

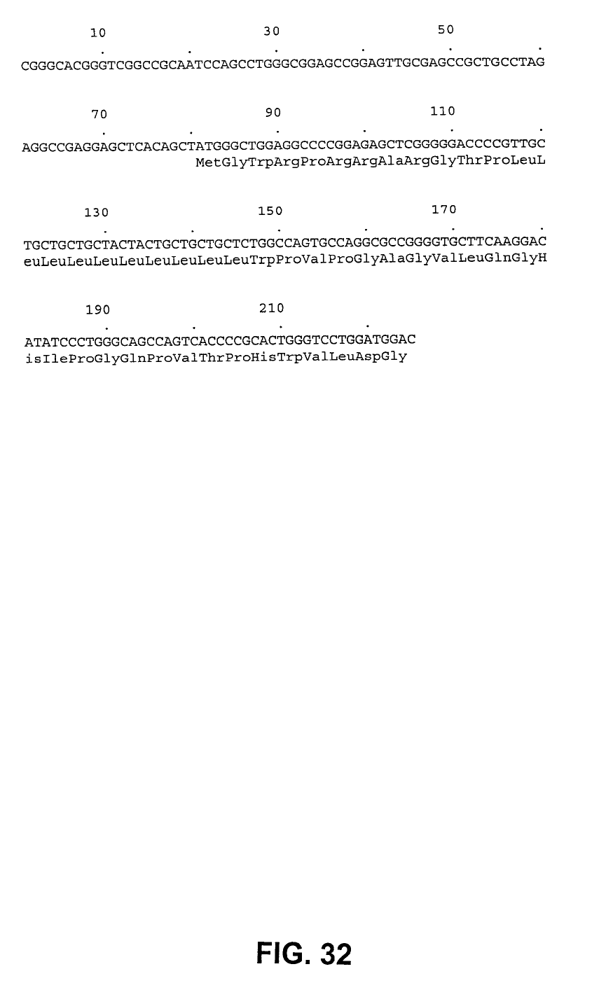

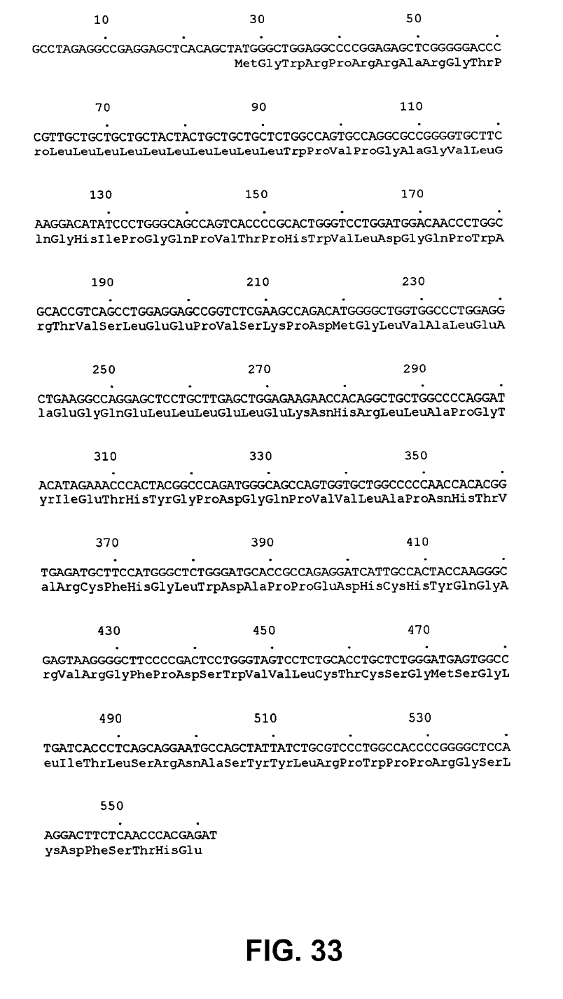

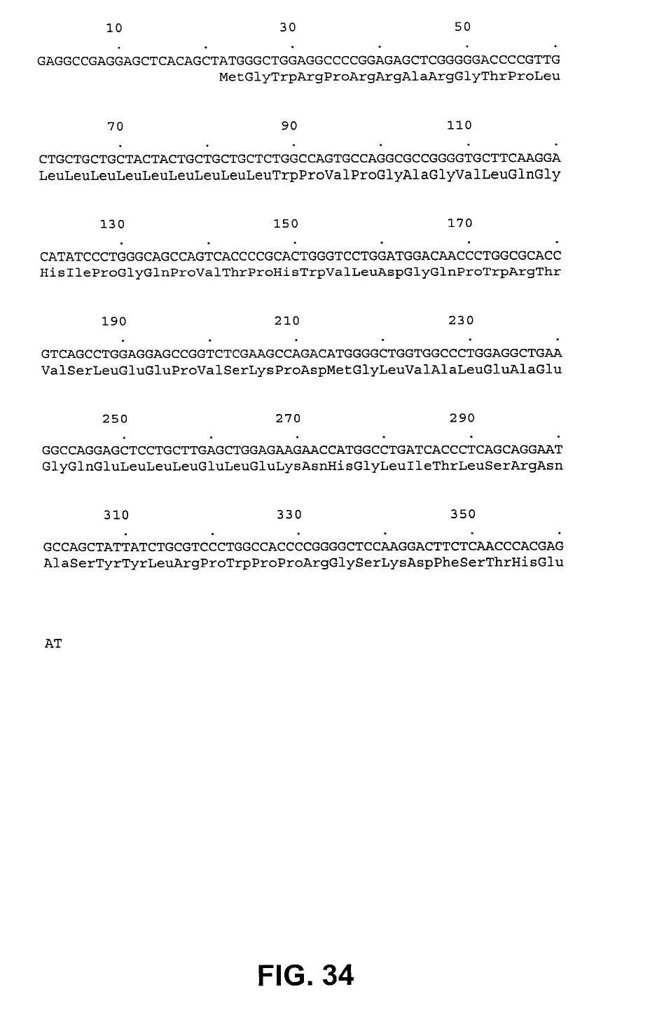

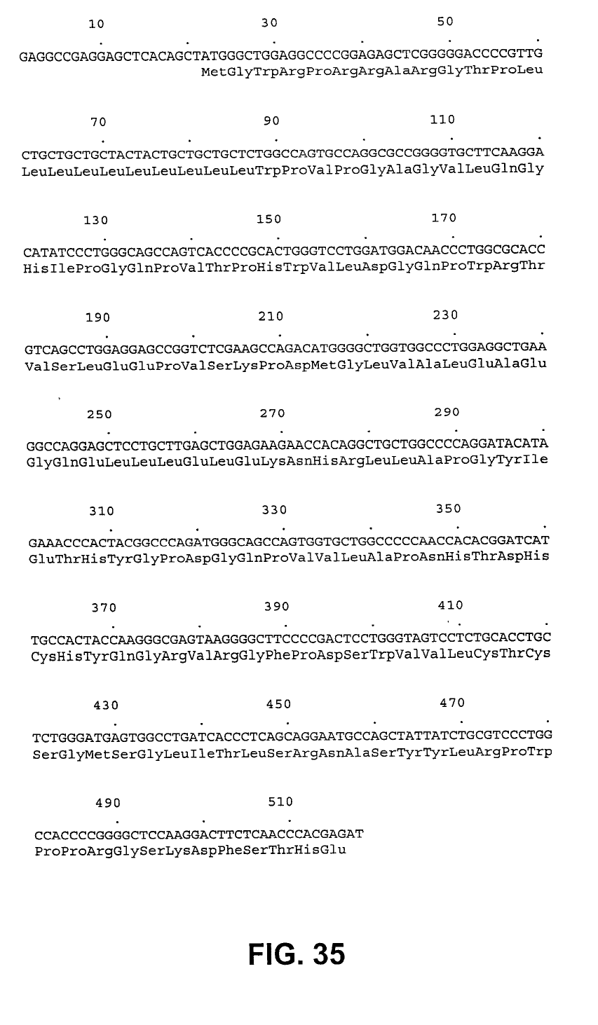

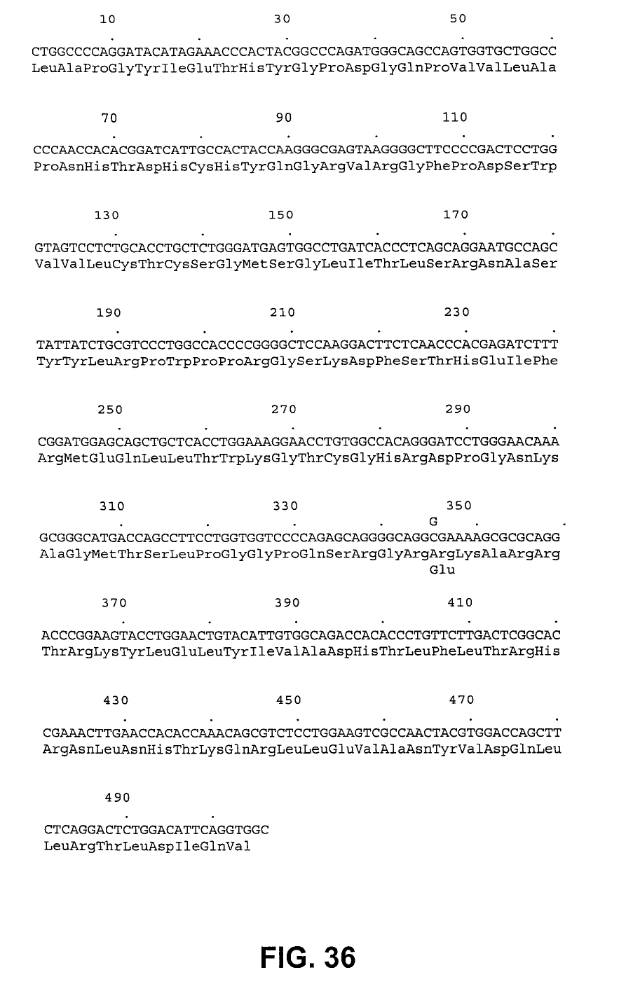

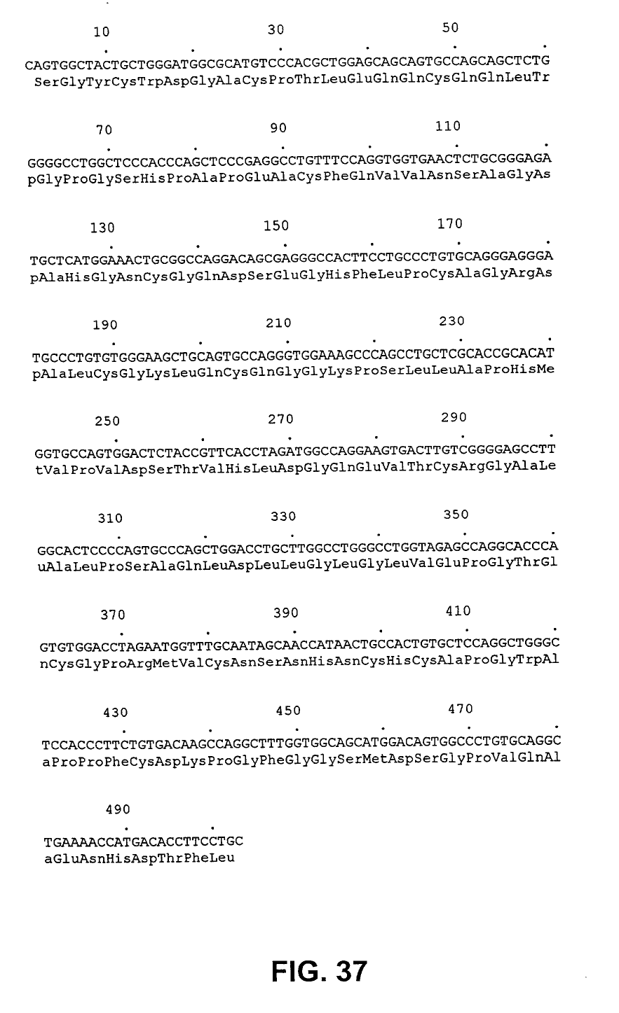

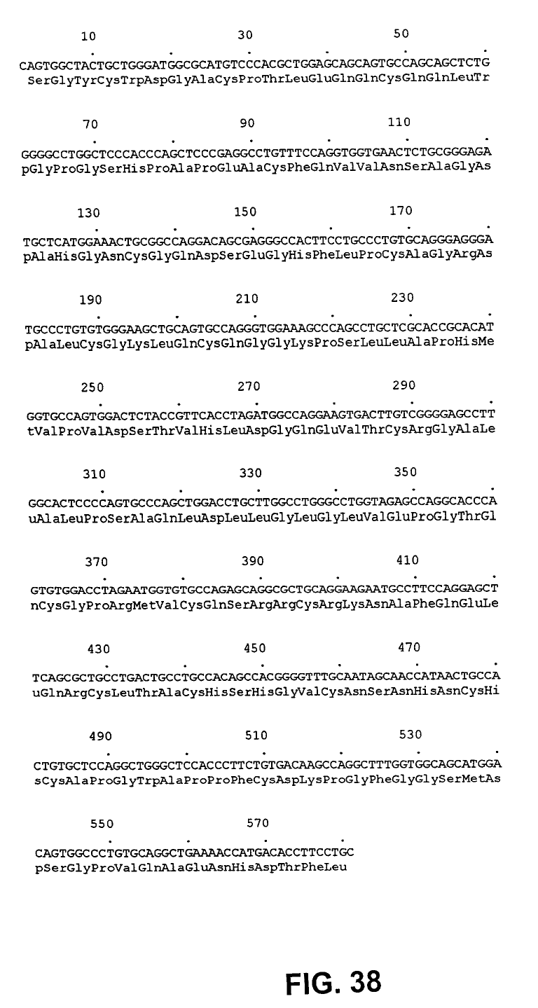

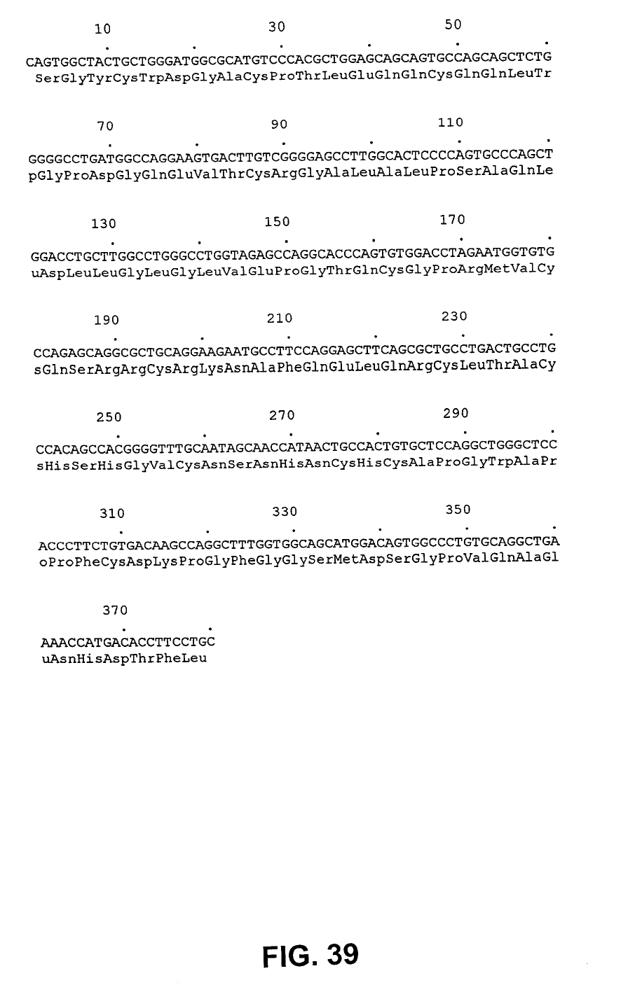

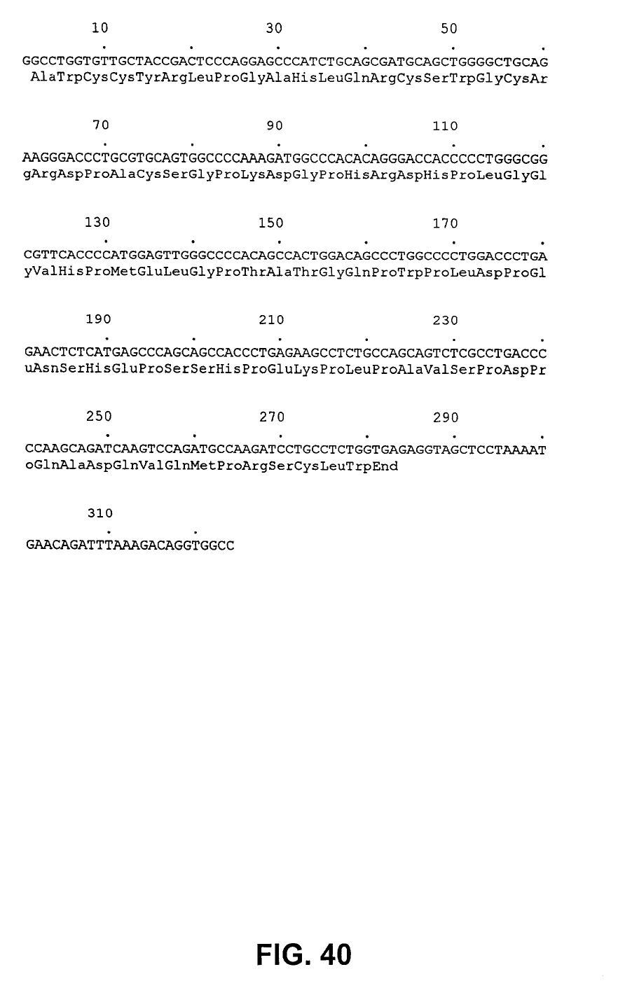

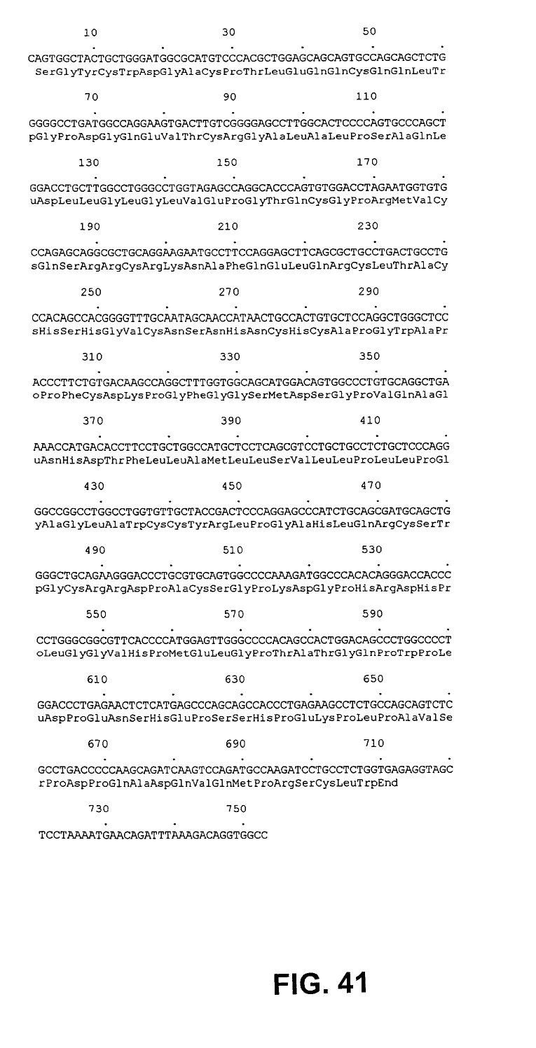

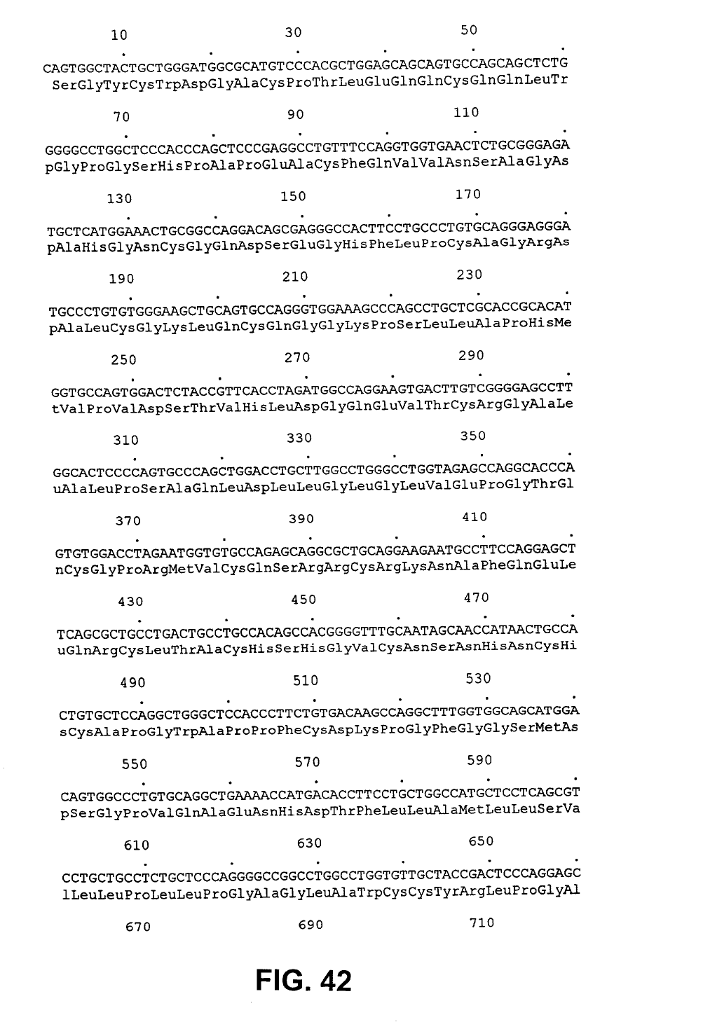

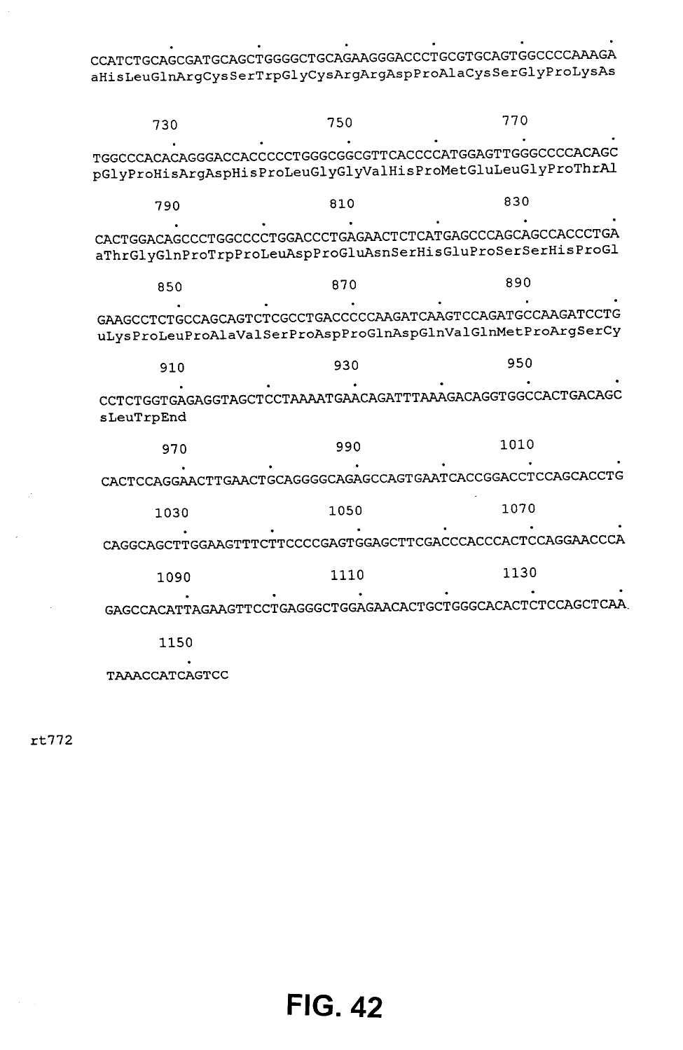

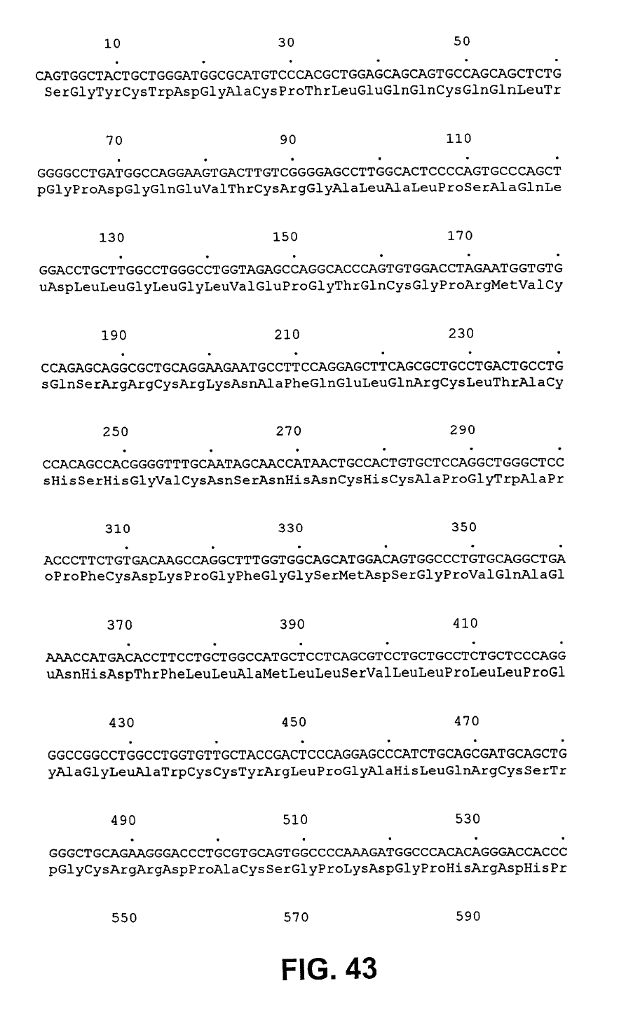

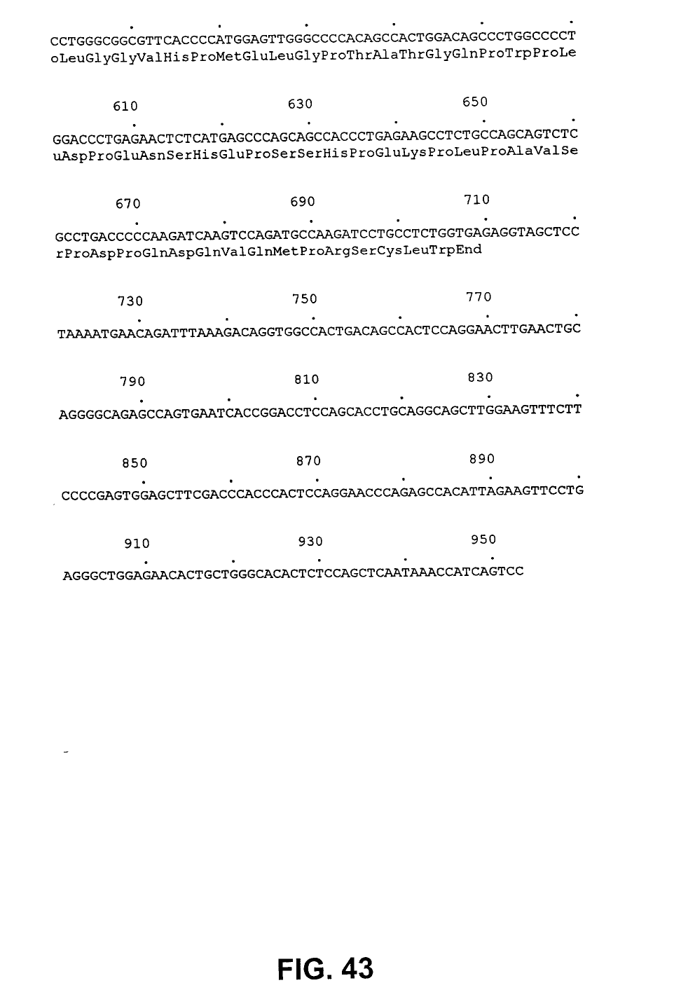

Through mapping analysis, BAC RPCI-11_{—}1098L22 (ATCC Designation No. PTA-3171) was identified as containing Gene 216. This BAC sequence (SEQ ID NO:5, FIG. 7) included the genomic sequence of Gene 216 (SEQ ID NO:6; FIG. 29), which corresponded to the cDNA sequence of Gene 216 (SEQ ID NO:1; FIG. 24).

### EXAMPLE 7

### Gene 216 cDNA Cloning and Expression Analysis

1. Construction and Screening of cDNA Libraries:

Directionally cloned cDNA libraries from normal lung and bronchial epithelium were constructed using standard methods (Soares et. al., 1994, Automated DNA Sequencing and Analysis, Adams et al. (eds), Academic Press, NY, pp. 110-114). Total and cytoplasmic RNAs were extracted from tissue or cells by homogenizing the sample in the presence of Guanidinium Thiocyanate-Phenol-Chloroform extraction buffer (e.g. Chomczynski and Sacchi, 1987, Anal. Biochem., 162:156-159) using a polytron homogenizer (Brinkman Instruments, http://www.brinkmann.com). Poly A+ RNA was isolated from total/cytoplasmic RNA using dynabeads-dT according to the manufacturer's recommendations (Dynal, Inc., http://www.dynal.com). The double stranded cDNA was then ligated into the plasmid vector pBluescript II KS+ (Stratagene, http://www.stratagene.com), and the ligation mixture was transformed into E. coli host DH10B or DH12S by electroporation (Soares, 1994). Following overnight growth at 37° C., DNA was recovered from the E coli colonies after scraping the plates by processing as directed for the Mega-prep kit (QIAGEN). The quality of the cDNA libraries was estimated by counting a portion of the total number of primary transformants, determining the average insert size, and the percentage of plasmids with no cDNA insert. Additional cDNA libraries (human total brain, heart, kidney, leukocyte, and fetal brain) were purchased from Life Technologies (Bethesda, Md.).

cDNA libraries, both oligo (dT) and random hexamer-primed, were used for isolating cDNA clones mapped within the disorder critical region. Four 10×10 arrays of each of the cDNA libraries were prepared as follows. The cDNA libraries were titered to 2.5×10⁶ using primary transformants. The appropriate volume of frozen stock was used to inoculate 2 L of LB/ampicillin (100 μg/μl). Four hundred aliquots containing 4 ml of the inoculated liquid culture were generated. Each tube contained about 5000 cfu (colony forming units). The tubes were incubated at 30° C. overnight with shaking until an OD of 0.7-0.9 was obtained. Frozen stocks were prepared for each of the cultures by aliquofting 300 μl of culture and 100 μl of 80% glycerol. Stocks were frozen in a dry ice/ethanol bath and stored at −70° C. DNA was isolated from the remaining culture using the QIAGEN spin mini-prep kit according to the manufacturer's instructions. The DNA from the 400 cultures were pooled to make 80 column and row pools. Markers were designed to amplify putative exons from candidate genes. Once a standard PCR condition was identified and specific cDNA libraries were determined to contain cDNA clones of interest, the markers were used to screen the arrayed library. Positive addresses indicating the presence of cDNA clones were confirmed by a second PCR using the same markers.

Once a cDNA library was identified as likely to contain cDNA clones corresponding to a transcript of interest from the disorder critical region, it was used to isolate a clone or clones containing cDNA inserts. This was accomplished by a modification of the standard “colony screening” method (Sambrook et al., 1989). Specifically, twenty 150 mm LB plus ampicillin agar plates were spread with 20,000 cfu of cDNA library. Colonies were allowed to grow overnight at 37° C. Colonies were then transferred to nylon filters (Hybond from Amersham-Pharmacia, or equivalent) and duplicates prepared by pressing two filters together essentially as described (Sambrook et al., 1989). The “master” plate was then incubated an additional 6-8 hr to allow the colonies additional growth. The DNA from the bacterial colonies was then bound to the nylon filters by treating the filters sequentially with denaturing solution (0.5 N NaOH, 1.5 M NaCl) for 2 min, and neutralization solution (0.5 M Tris-Cl pH 8.0, 1.5 M NaCl) for 2 min (twice). The bacterial colonies were removed from the filters by washing in a solution of 2× SSC/2% SDS for 1 min while rubbing with tissue paper. The filters were air-dried and baked under vacuum at 80° C. for 1-2 hr to crosslink the DNA to the filters.

cDNA hybridization probes were prepared by random hexamer labeling (Fineberg and Vogelstein, 1983, Anal. Biochem., 132:6-13) or by including gene-specific primers and no random hexamers in the reaction (for small fragments). The colony membranes were then pre-washed in 10 mM Tris-Cl pH 8.0, 1 M NaCl, 1 mM EDTA, 0.1% SDS for 30 min at 55° C. Following the pre-wash, the filters were pre-hybridized in >2 ml/filter of 6× SSC, 50% deionized formamide, 2% SDS, 5× Denhardt's solution, and 100 mg/ml denatured salmon sperm DNA, at 42° C. for 30 min. The filters were then transferred to hybridization solution (6× SSC, 2% SDS, 5× Denhardt's, 100 mg/ml denatured salmon sperm DNA) containing denatured α-32P-dCTP-labeled cDNA probe and incubated overnight at 42° C.

The following morning, the filters were washed under constant agitation in 2× SSC, 2% SDS at room temperature for 20 min, followed by two washes at 65° C. for 15 min each. A second wash was performed in 0.5 X SSC, 0.5% SDS for 15 min at 65° C. Filters were then wrapped in plastic wrap and exposed to radiographic film. Individual colonies on plates were aligned with the autoradiograph and positive clones picked into a 1 ml solution of LB Broth containing ampicillin. After shaking at 37° C. for 1-2 hr, aliquots of the solution were plated on 150 mm plates for secondary screening. Secondary screening was identical to primary screening (above) except that it was performed on plates containing ˜250 colonies so that individual colonies could be clearly identified. Positive cDNA clones were characterized by restriction endonuclease cleavage, PCR, and direct sequencing to confirm the sequence identity between the original probe and the isolated clone.

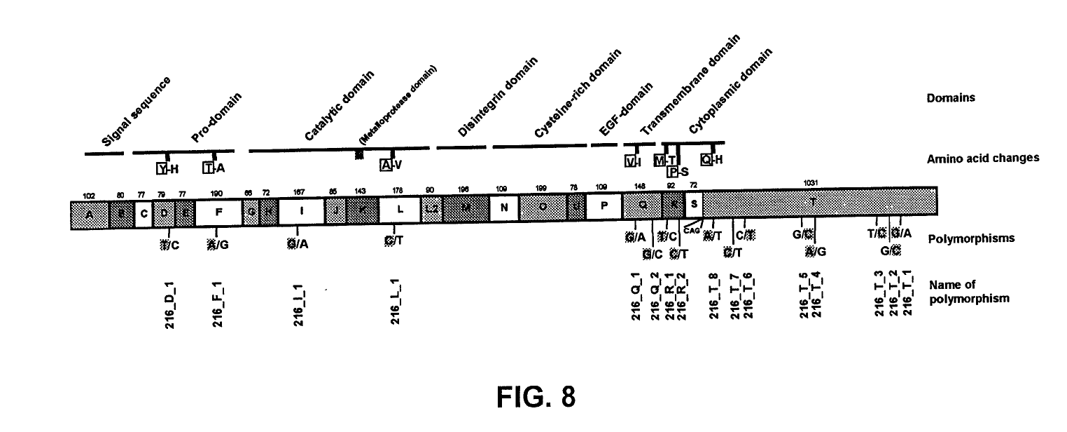

To obtain the full-length cDNA, novel sequence from the 5′-end of the clone was used to reprobe the library. This process was repeated until the length of the cDNA cloned matched that of the mRNA, estimated by Northern analysis. Utilizing this process, a single uterus clone was isolated and deposited as clone Gene 216_CS759 with the American Type Culture Collection (ATCC), 10801 University Blvd., Manassas, Va. 20110-2209 USA, under ATCC Designation No. PTA-3173, on Mar. 14, 2001, according to the terms of the Budapest Treaty. The uterus clone (SEQ ID NO:3) contained the entire Gene 216 open reading frame. Both strands of this clone were completely sequenced and the data were compared against the BAC sequence. Any discrepancies were flagged, and these regions were resequenced. The final analysis of the sequence revealed that the uterine clone was 3433 bp long and contained the full complement of exons defining the open reading frame (SEQ ID NO:3). In addition, the clone contained a small portion of the 5′ untranslated region (5 bp), the entire 3′ untranslated region with a polyadenylation signal, and a poly A tail of 76 bp in length. The Gene 216 open reading frame was determined to be 2436 bp in length and to encode a protein of 812 amino acids (SEQ ID NO:363). Analysis of the composition of SNPs across the cDNA clone revealed that it contained the most frequent haplotype (FIG. 8, see below).

Rapid Amplification of cDNA ends (RACE) was performed following the manufacturer's instructions using a Marathon cDNA Amplification Kit (CLONTECH) as a method for cloning the 5′ and 3′ ends of candidate genes. cDNA pools were prepared from total RNA by performing first strand synthesis. For first strand synthesis, a sample of total RNA sample was mixed with a modified oligo (dT) primer, heated to 70° C., cooled on ice and incubated with: 5× first strand buffer (CLONTECH), 10 mM dNTP mix, and AMV Reverse Transcriptase (20 U/μl). The reaction mixture was incubated at 42° C. for 1 hr and placed on ice. For second-strand synthesis, the following components were added directly to the reaction tube: 5×second-strand buffer (CLONTECH), 10 mM dNTP mix, sterile water, and 20×second-strand enzyme cocktail (CLONTECH). The reaction mixture was incubated at 16° C. for 1.5 hr. T4 DNA Polymerase was added to the reaction mixture and incubated at 16° C. for 45 min. The second-strand synthesis was terminated with the addition of an EDTA/Glycogen mix. The sample was purified by phenol/chloroform extraction and ammonium acetate precipitation. The cDNA pools were checked for quality by analyzing on an agarose gel for size distribution. Marathon cDNA adapters were then ligated onto the cDNA ends. The specific adapters contained priming sites that allowed for amplification of either 5′ or 3′ ends, and varied depending on the orientation of the gene specific primer (GSP) that was chosen. An aliquot of the double stranded cDNA was added to the following reagents: 10 μM Marathon cDNA adapter, 5× DNA ligation buffer, T4 DNA ligase. The reaction was incubated at 16° C. overnight and heat inactivated to terminate the reaction. PCR was performed by the addition of the following to the diluted double stranded cDNA pool: 10× cDNA PCR reaction buffer, 10 μM dNTP mix, 10 μM GSP, 10 μM AP1 primer (kit), 50× Advantage cDNA Polymerase Mix. Thermal Cycling conditions were carried out at 94° C. for 30 sec; 5 cycles of 94° C. for 5 sec, 72° C. for 4 min, 5 cycles of 94° C. for 5 sec, and 70° C. for 4 min; 23 cycles of 94° C. for 5 sec; 68° C. for 4 min. The first round of PCR was performed using the GSP to extend to the end of the adapter to create the adapter primer-binding site. Following this, exponential amplification of the specific cDNA of interest was performed. Usually, a second, nested PCR was performed to provide specificity. The RACE product was analyzed on an agarose gel. Following excision from the gel and purification (GeneClean, BIO 101), the RACE product was then cloned into pCTNR (General Contractor DNA Cloning System, 5′-3′, Inc.) and sequenced to verify that the clone was specific to the gene of interest.

The 5′ RACE technique was employed to identify the 5′ untranslated region of Gene 216. Experiments were performed using lung mRNA and a primer that hybridized near the 5′ end of the available sequence. The result of the experiment identified an additional 75 bp 5′ of that present in the uterus cDNA clone (rt690; SEQ ID NO:351). This sequence was subsequently cloned and deposited with the ATCC (American Type Culture Collection, 10801 University Blvd., Manassas, Va. 20110-2209 USA), as clone Gene 216_rt690, under ATCC Designation No.PTA-3172 on Mar. 14, 2001, according to the terms of the Budapest Treaty.

Further attempts to extend the 5′ end of Gene 216 by 5′ RACE gave similar results indicating that the 5′ end of the transcript was obtained.

This sequence in combination with the uterus cDNA clone yielded the master consensus sequence containing the 5′ to 3′ cDNA for Gene 216 (SEQ ID NO:1; FIG. 24).

2. Identification of Splice Variants:

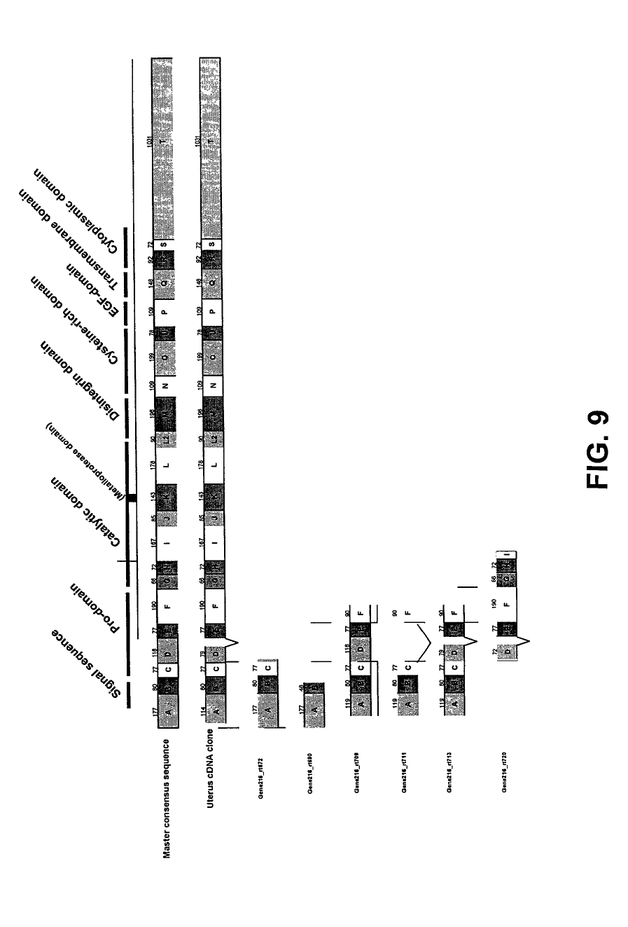

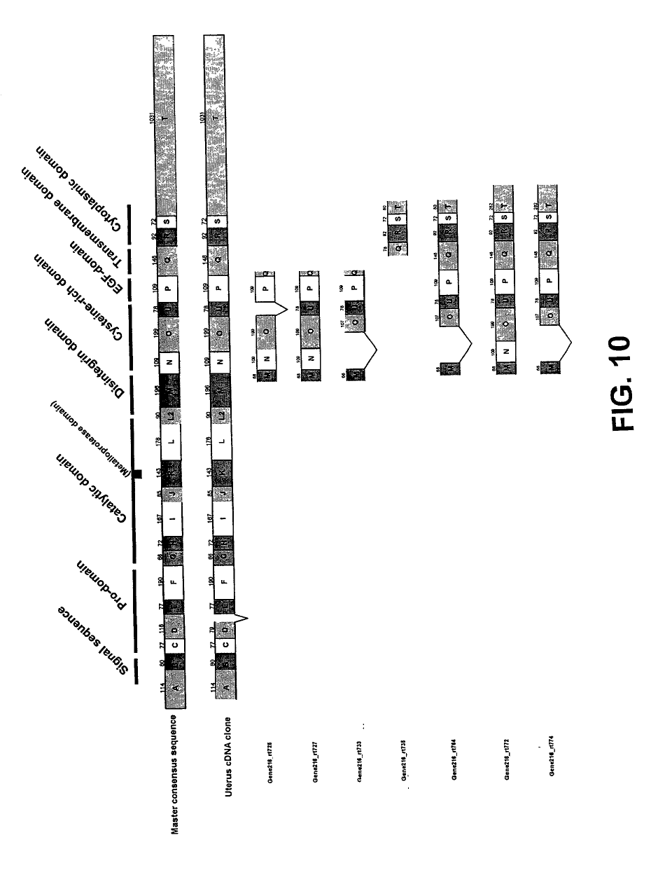

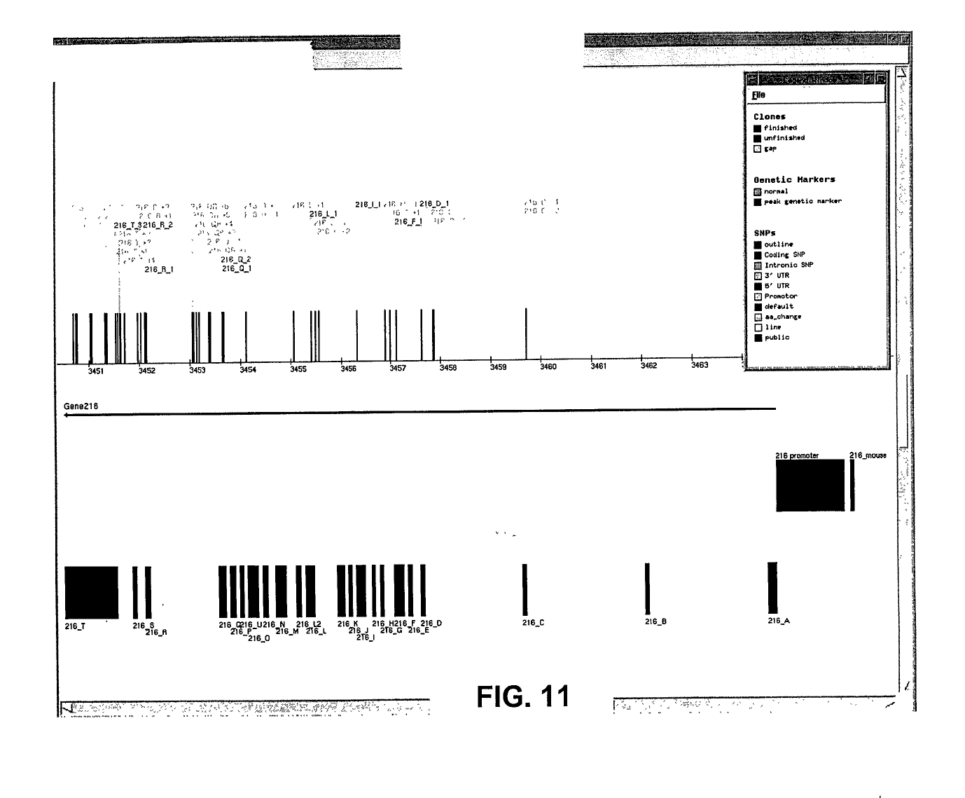

Additional cDNA clones were isolated that represented alternatively spliced variants of Gene 216. To ensure that all splice variants present in lung tissue were identified, an RT-PCR-based screening protocol was designed using multiple primer pairs spanning the entire gene. These amplicons produced PCR fragments of approximately 600 bp and overlapped by approximately 100 bp. The PCR products were fractionated on agarose gels and any fragments that were different from the expected size were cloned and sequenced. These results are summarized in FIGS. 9 and 10. The availability of the complete genomic sequence of BAC RPCI-11_{—}1098L22 enabled the intron/exon structure of Gene 216 (FIG. 11) to be determined. Gene 216 contains 21 exons that span approximately 23.5 kb of genomic DNA.

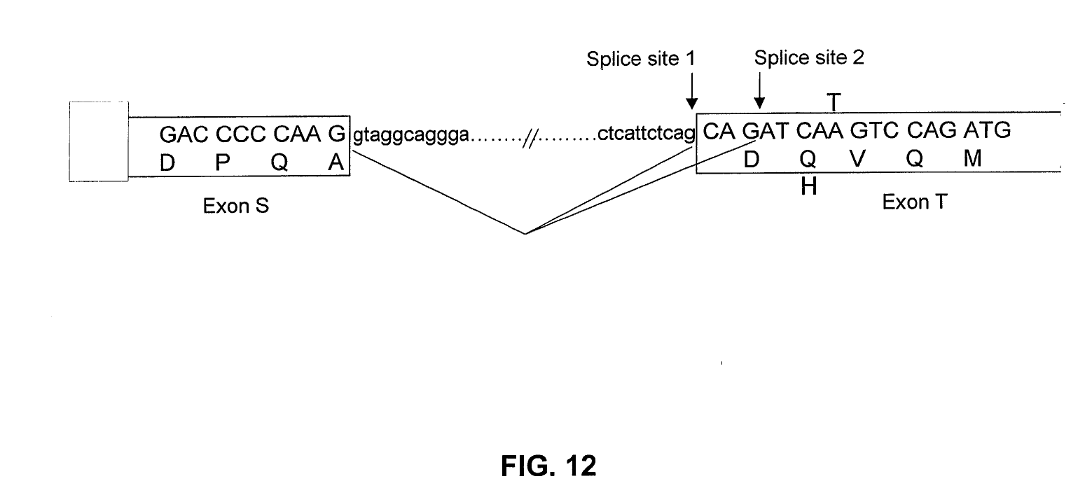

Analysis of the sequence surrounding the intron/exon boundaries indicated that the consensus splice sequence GT/AG was upheld in all cases (Table 4). However, in several of the cDNA clones, an alternative use of a splice site at the intron/exon boundary of exon T was identified. The sequence CAGCAG was present at the border of intron ST and exon T resulting in a duplication of the canonical acceptor splice consensus CAG. Typically, a C residue preceding the AG is found in approximately 65% of acceptor splice sites. As a consequence, the splicing machinery can utilize either AG resulting in the presence or absence of an alanine. If the first AG (splice site 1) were utilized near the junction of intron ST and exon T, the resulting protein would encode the amino acid sequence DPQADQVQM (FIG. 12) (SEQ ID NO:60). However, if the second AG (splice site 2) were favored, then one alanine would be omitted from the amino acid sequence and the protein would contain the amino acid sequence DPQDQVQM (FIG. 12) (SEQ ID NO:61). The percentage that used splice site 1 or splice site 2 could not be determined from the dataset because the majority of the clones were derived from PCR-based techniques.

TABLE 4

| | | | | |
|---|---|---|---|---|
| EXON | 3′ INTRON | 5′ EXON | 3′ EXON | 5′ INTRON |
| A | | | AAG | GTGAGG |
| B | CAG | GAC | CCG | GTCAGT |
| C | GAG | GTC | CCA | GTGAGT |
| D | CAG | CAG | ACG | GTGAGA |
| D (ALT) | CAG | CAG | GAG | GTACCC |
| E | TAG | GAT | GAG | GTGAGG |
| F | TAG | TGG | AGG | GTCAGG |
| G | CAG | GGC | CTG | GTGAGG |
| H | CAG | TTC | CAG | GTTGGG |
| I | CAG | CTT | CAC | GTGGGT |
| J | CAG | GGG | ACG | GTGAGC |
| K | CAG | GAC | CGG | GTACGC |
| L | TAG | GCA | CAG | GTTAAG |
| L2 | CAG | GAG | CTG | GTGAGG |
| M | CAG | CTG | CTG | GTGAGA |
| N | CAG | GCT | GAG | GTAGGG |
| O | CAG | GGA | ATG | GTGAGC |
| O (ALT) | TAG | ATG | ATG | GTGAGC |
| U | TAG | GTG | GGG | GTGAGA |
| P | CAG | GTT | AAA | GTATGC |
| Q | CAG | AGC | TGG | GTAGGC |
| R | CAG | CCC | TGG | GTGAGT |
| S | CAG | ACC | AAG | GTAGGC |
| T | GAG | CAG | | |

C₆₅ A₁₀₀ G₁₀₀ N A₆₄ G₇₃ G₁₀₀ T₁₀₀ A₆₂ A₆₈ G₈₄ T₆₃

3. Promoter Analysis:

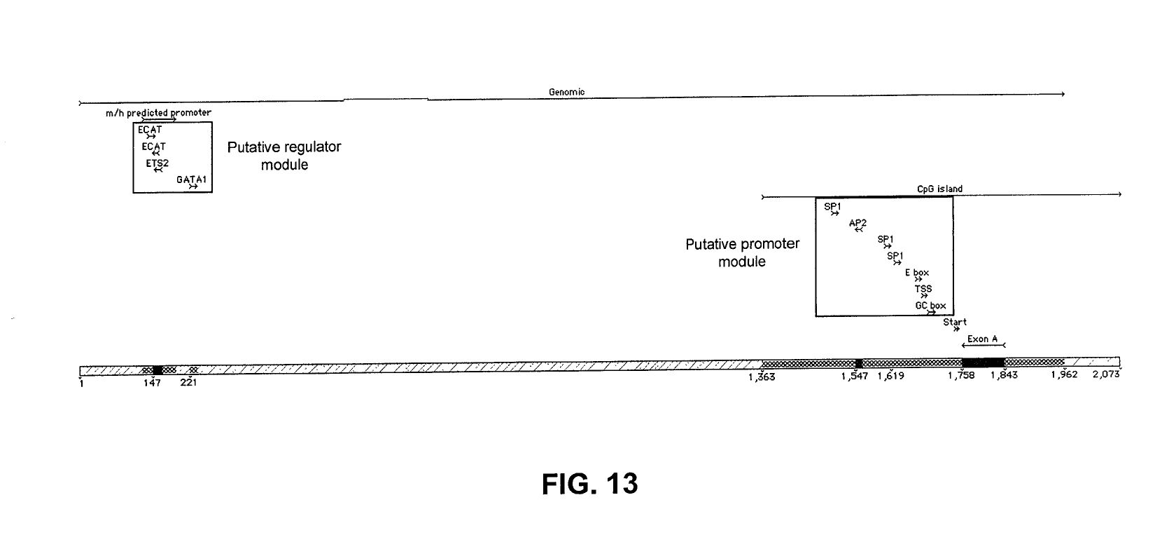

In order to identify the transcriptional start site of Gene 216, multiple 5′ RACE products were sequenced from several different tissues. In most cases the 5′ ends were located 80 bp upstream of the translational start site. The region upstream of this sequence was then analyzed for potential transcription factor binding sites using GEMS Launcher, a promoter analysis program (http://anthea.gsf.de/). GEMS Launcher uses statistically weighted algorithms to identify binding elements that comprise a promoter or regulatory module. A stretch of DNA sequence spanning the 2000 bp upstream of the translational start site was analyzed. The results indicated that Gene 216 did not possess a TATA or CCMT box. In fact, the first binding element that was identified was a GC box within the 5′ untranslated region oriented in the opposite direction (FIG. 13). This result is not unprecedented since 60% of TATA-less genes possess a GC box on the opposing strand. Also, this result was in agreement with published data regarding the promoters of mouse ADAM 17 and 19. Other binding elements that were identified within 600 bp upstream of the initiator methionine included an E-box, one AP2, and three SP1 sites (FIG. 13). These types of binding elements were also identified in the mouse ADAM 17 and 19 genes and may represent components of a promoter module for Gene 216. Approximately 1200 bp upstream of the putative promoter module, GEMS Launcher identified binding elements that may comprise an additional regulatory element (FIG. 13). This region was highly conserved with the mouse ortholog of Gene 216 (see below), as determined by dot matrix analysis.

4. BLAST Analysis:

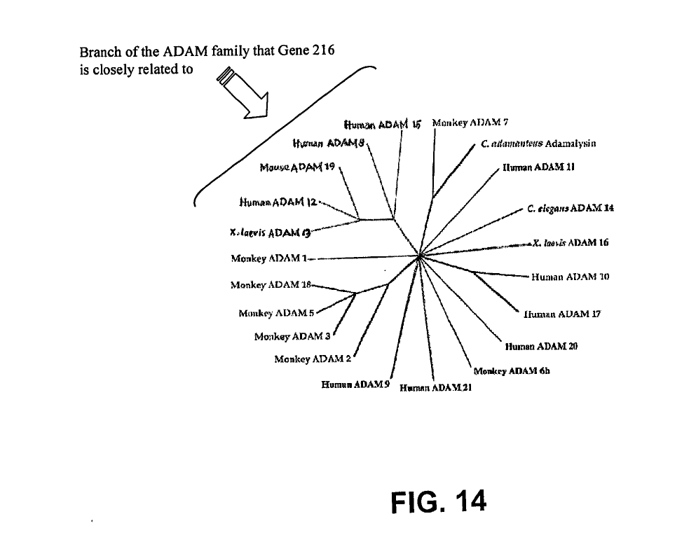

BLASTP, BLASTN, and BLASTX analysis of Gene 216 against protein and nucleotide databases revealed that it was a novel member of the ADAM (A Disintegrin And Metalloprotease) gene family. This gene family, of which there are currently 31 members, is a sub-group of the zinc-dependent metalloprotease superfamily. ADAMs have a complex domain organization that includes a signal sequence, propeptide, metalloprotease, disintegrin, cysteine-rich, and epidermal growth factor-like domains, as well as a transmembrane region and cytoplasmic tail. ADAM proteins have been implicated in many processes such as proteolysis in the secretory pathway and extracellular matrix, extra- and intra-cellular signaling, processing of plasma membrane proteins and procytokine conversion. The homology of Gene 216 and human ADAMs 19, 12, 15, 8 and 9 indicated that Gene 216 belonged to a branch of the 31-member family containing active metalloprotease domains (FIG. 14).

6. Expression Analysis:

To characterize the expression of Gene 216, a series of expression experiments were performed.

i. Northern Analysis:

To characterize novel genes, Northern analysis (Sambrook et al., 1989) can be used to determine the length, in nucleotides, of the processed transcript or messenger RNA (mRNA). Probes were generated using one of the methods described below. Briefly, sequence verified IMAGE consortium cDNA clones were digested with appropriate restriction endonucleases to release the insert. The restriction digest was electrophoresed on an agarose gel and the bands containing the insert were excised. The gel piece containing the DNA insert was placed in a Spin-X (Corning Costar Corporation, Cambridge, Mass.) or Supelco spin column (Supelco Park, Pa.) and spun at high speed for 15 min. The DNA was ethanol precipitated and resuspended in TE. Alternatively, PCR products obtained from genomic DNA or RT-PCR were purified. First, oligonucleotide primers were designed for use in the polymerase chain reaction (PCR) so that portions of the cDNA, EST, or genomic DNA could be amplified from a pool of DNA molecules or RNA population (RT-PCR). The PCR primers were used in a reaction containing genomic DNA to verify that they generated a product of the predicted size (based on the genomic sequence. Inserts purified from IMAGE clones or PCR products were random primer labeled (Fineberg and Vogelstein, supra) to generate probes for hybridization. Probes from purified PCR products were generated by incorporation of a-³²P-dCTP in second round of PCR. Commercially available Multiple Tissue Northern blots (CLONTECH) were hybridized and washed under conditions recommended by the manufacturer. A separate filter that contained 6 tissues from the immune system was also utilized. The results revealed a major 5.0 kb transcript and a minor 3.5 kb transcript that were expressed in most tissues examined (FIGS. 15A-15B). The strongest signals were consistently identified in heart, skeletal muscle, colon, lymph, and small intestine, with lung, liver, kidney, placenta, bone marrow, and brain showing moderate expression levels.

The 5 kb transcript was further analyzed to determine if it was an incompletely spliced version of the Gene 216 transcript. To test this hypothesis, Northern blotting was performed using cytoplasmic mRNA isolated from bronchial smooth muscle cells. The same radioactive probe was employed as previously. The results showed a very strong 3.5 kb signal and no signal at 5.0 kb (FIG. 15C) suggesting that the predominant 5 kb transcript contained intronic material and was localized to the nucleus. Interestingly, intron QR is 1.4 kb in size. The addition of the QR intron and the 3.5 kb full length cDNA would total ˜5.0 kb. Accordingly, there may be regulatory elements within the region around intron QR that affect splicing, retention in the nucleus, and/or transport to the cytoplasm.

ii. RNA Dot Blot Analysis:

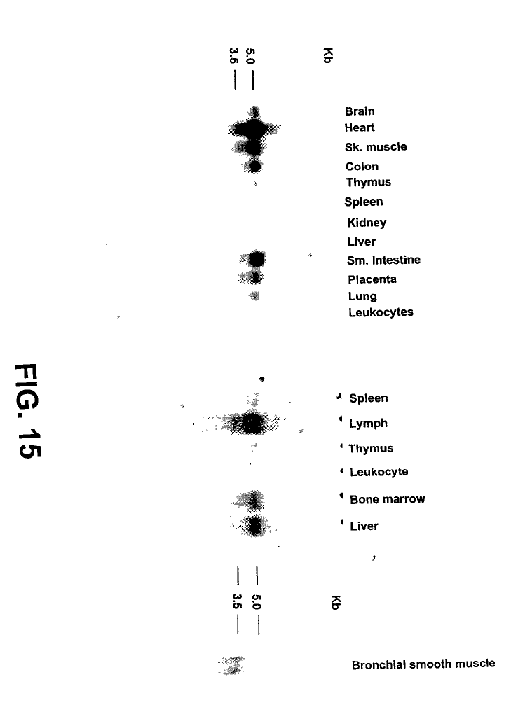

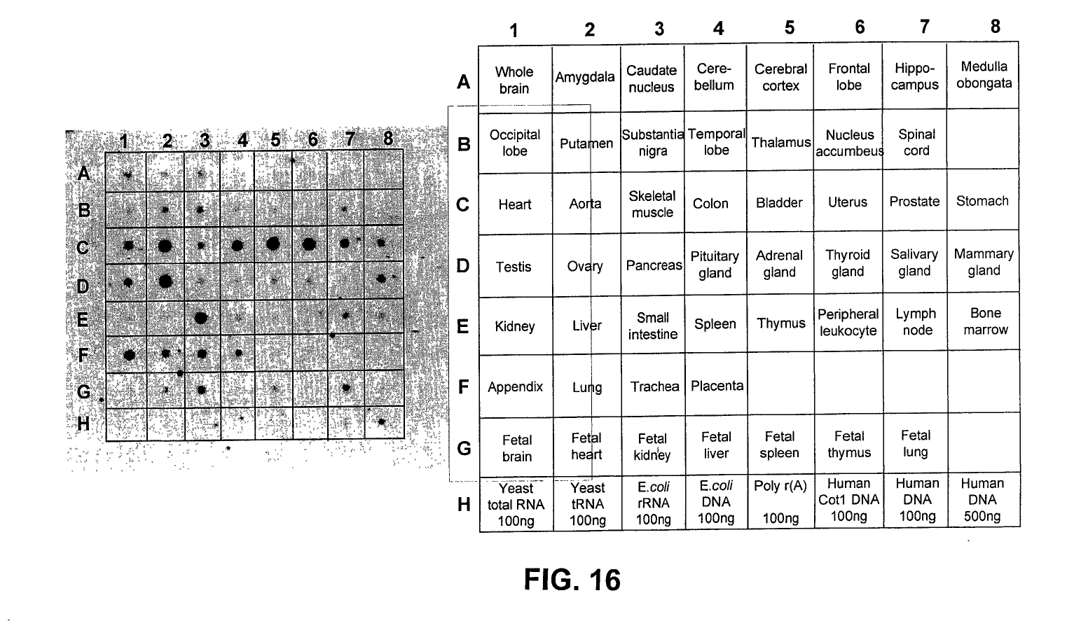

RNA dot blotting was used to determine the expression of Gene 216 in a wide range of tissues. mRNA from 50 tissues was dotted onto a nylon filter, and a radioactive probe designed to hybridize to the 3′ untranslated region was used. FIG. 16 shows that Gene 216 was highly expressed in gastrointestinal tissues as well as aorta, uterus, prostate, ovary, lung, fetal lung, trachea and placenta. Notably, the majority of these tissues are derived from the endoderm, which forms a tube that produces the primordium of the digestive tract. Extensions from this wall also develop into organs such as the lung and trachea.

iii. RT-PCR:

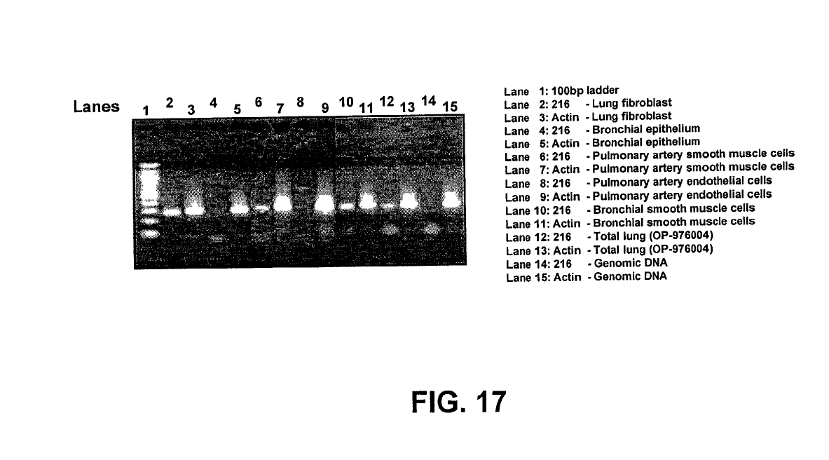

Total RNA isolated from primary cultures of seven cell types cultured from lung tissue was analyzed in RT-PCR experiments. Genomic DNA was removed from the total RNA by DNasel digestion. The “Superscript” Preamplification System for First strand cDNA synthesis” (Life Technologies) was used according to manufacturer's specifications with oligo(dT) or random hexamers to synthesize cDNA from the DNasel treated total RNA. Gene specific primers were used to amplify the target cDNAs in a 30 μl PCR reaction containing 0.5 μl of first strand cDNA, 1 μl sense primer (10 μM), 1 μl antisense primer (10 μM), 3 μl dNTPs (2 mM), 1.2 μl MgCl₂ (25 mM), 3 μl 10× PCR buffer and 1 unit of Taq Polymerase (Perkin Elmer). The PCR reaction was initially incubated at 94° C. for 4 min, followed by 30 cycles of incubation at 94° C. for 30 sec, 58° C. for 1 min, and 72° C. for 1 min; then followed by a final incubation at 72° C. for 7 min. PCR products were analyzed on agarose gels. FIG. 17 shows that Gene 216 was expressed in lung fibroblasts, pulmonary artery smooth muscle cells, bronchial smooth muscle cells and total lung, but not in bronchial epithelium or pulmonary artery endothelial cells.

iv. cDNA Library Representation:

A comprehensive approach to determining the tissue distribution of Gene 216 was performed in silico by mining the public EST database and Genome Therpaeutics Corporation's internal cDNA database. BLAST analysis identified ESTs from multiple cDNA libraries. A summary of all tissues expressing Gene 216 is given in Table 5.

TABLE 5

| Source | Tissue |
|---|---|
| UNIGENE | Eye |
| | Muscle |
| | Placenta |
| | Stomach |
| | Uterus |
| | Whole embryo |
| | Breast |
| | Normal testis |
| Direct selected cDNAs | Bronchial smooth muscle (1 clone) |
| | Normal lung (2 clones) |
| | Brain (1 clone) |
| Primary cell types (RT/PCR) | Pulmonary artery smooth muscle |
| | Bronchial smooth muscle |
| | Lung fibroblast |
| | Total lung |
| RNA Dot Blot | Aorta |
| | Colon |
| | Bladder |
| | Uterus |
| | Prostate |
| | Ovary |
| | Small intestine |
| | Heart |
| | Stomach |
| | Testis |
| | Appendix |
| | Lung |
| | Trachea |
| | Fetal kidney |
| | Fetal lung |
| Northern Blot | Brain |
| | Heart |
| | Skeletal muscle |
| | Colon |
| | Thymus |
| | Spleen |
| | Kidney |
| | Liver |
| | Small intestine |
| | Placenta |
| | Lung |
| | Lymph |
| | Bone marrow |

### EXAMPLE 8

### Gene 216 Polypeptide

1. ADAM Family Features:

The zinc-dependent metalloprotease superfamily is comprised of several sub-groups. Those proteases that exhibit the characteristic Zn-binding consensus sequence HEXXHXXGXXH (SEQ ID NO:62) are referred to as zincins. The 3 histidines play an essential role in binding to the catalytically essential zinc ion. The zincins can be further classified into metzincins if a methionine residue is located beneath the active-site zinc ion (“Met-turn” motif). Within this sub-group there are 4 sub-families: astacins, matraxins, adamlysins, and serralysins. The ADAM genes fall within the adamlysins sub-family along with snake venom metalloproteases.

Currently, there are 31 members of the ADAM family. The ADAM genes encode proteins of approximately 750 amino acids with 8 different domains. Domain I is a pre-domain and contains the signal sequence peptide that facilitates secretion through the plasma membrane. Domain II is a pro-domain that is cleaved before the protein is secreted resulting in activation of the catalytic domain. Domain III is a catalytic domain containing metalloprotease activity. Domain IV is a disintegrin-like domain and is believed to interact with integrins or other receptors. Domain V is a cysteine-rich domain and is speculated to be involved in protein-protein interactions or in the presentation of the disintegrin-like domain. Domain VI is an EGF-like domain that plays a role in stimulating membrane fusion. Domain VII is a transmembrane domain that anchors the ADAM protein to the membrane. Domain VIII is a cytoplasmic domain and contains binding sites for cytoskeletal-associated proteins and/or SH3 binding domains that may play a role in bi-directional signaling. See FIG. 8 for the location of ADAM domains identified in the Gene 216 protein sequence.

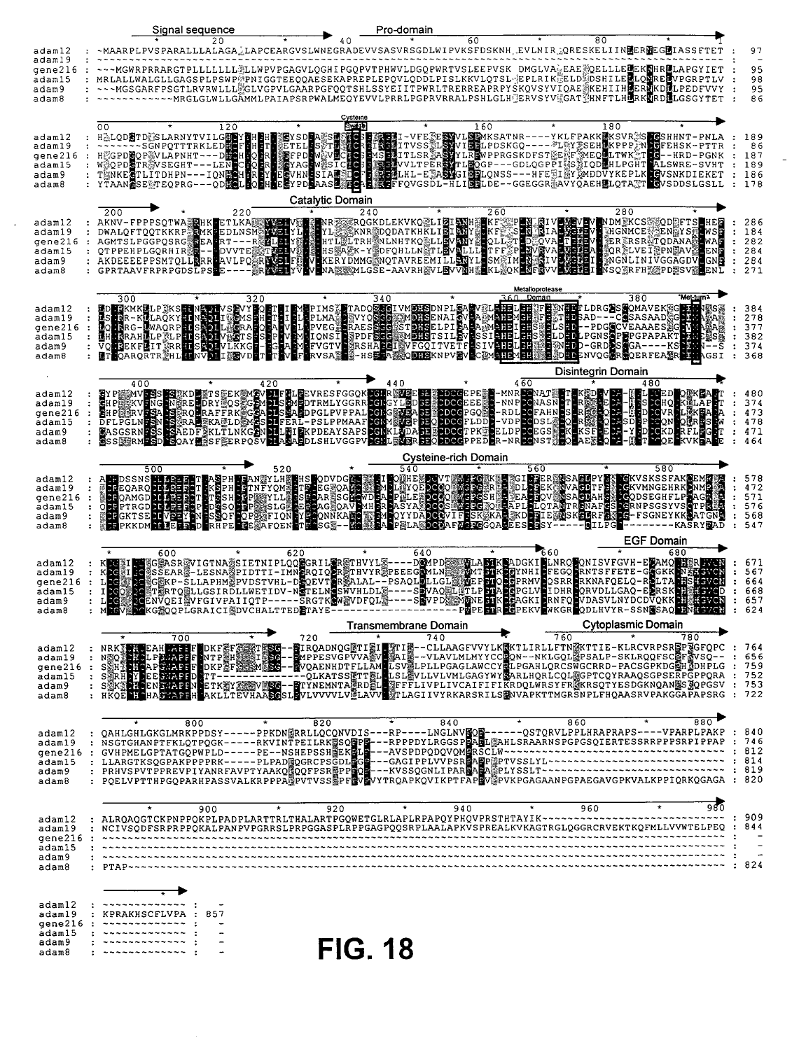

To determine whether Gene 216 was a novel member of the ADAM family, the 812 amino acid sequence was aligned by Pile-Up (Genetics Computer Group, http://www.gcg.com) (FIG. 18). These analyses indicated that Gene 216 possessed the characteristic consensus sequence HEXXHXXGXXH (SEQ ID NO:62) located within the catalytic domain. In addition, a methionine residue referred to as a “Met-turn” was identified in the Gene 216 protein. A conserved cysteine (amino acid 133 in Gene 216) that plays a role in activating ADAM proteins was identified in the prodomain of Gene 216 protein. In ADAM proteins, this single cysteine residue forms an intramolecular complex with the zinc ion bound to the metalloprotease domain and blocks the active site. The catalytic domain is activated by the dissociation of the cysteine from the complex, resulting in either a conformational change or enzymatic cleavage of the prodomain. This process is referred to as the “cysteine switch”.

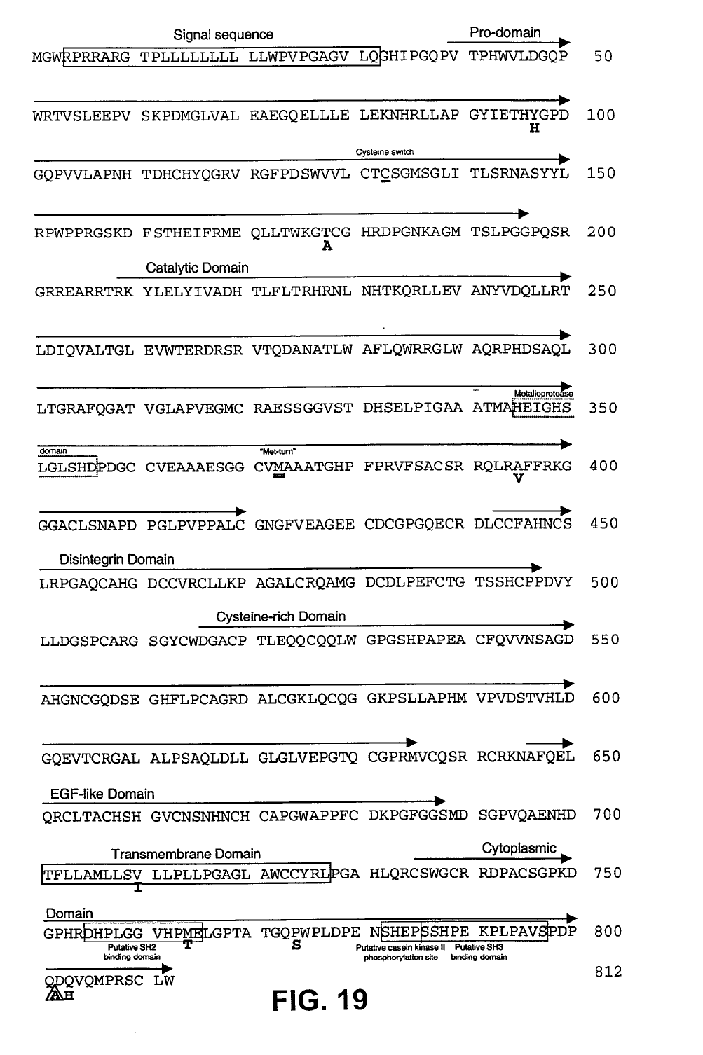

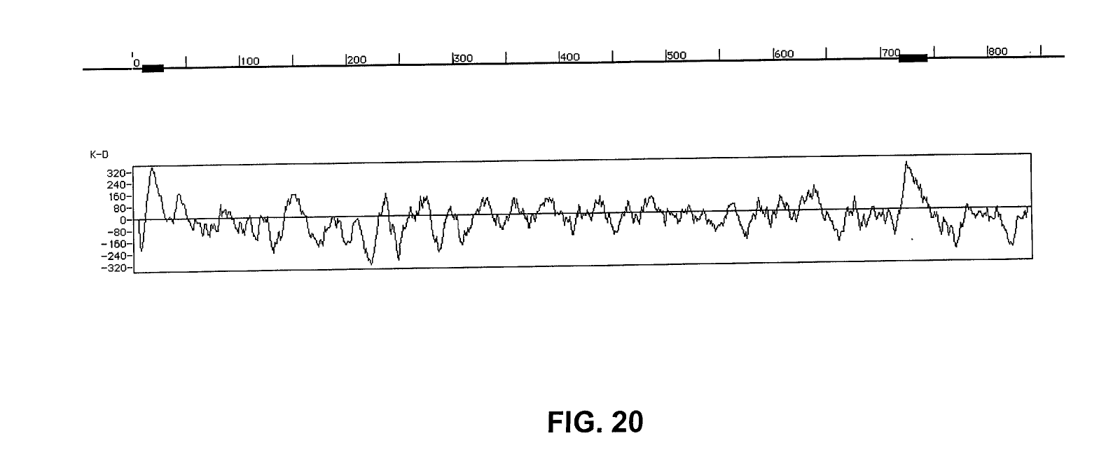

In ADAM 12, the position of the cysteine residue was reported to be located in a different position in the prodomain (B. L. Gilpin et al., 1998, J. Biol. Chem. 273:157-166). This location would correspond to the cysteine residue at amino acid 179 in Gene 216 (FIG. 19). However, in accordance with analyses performed by Stone et al., using 14 ADAMs, including ADAMs 8, 9, 12 and 15, the cysteine residue corresponding to position 133 of Gene 216 (FIGS. 18 and 19) was identified as being involved in the “cysteine switch”. In addition, there appeared to be more sequence identity around the cysteine at amino acid 133 in Gene 216 than at position 179. This provided further support that the cysteine at position 133 was involved in the “cysteine switch”. The alignment also indicated that the amino acid sequence of Gene 216 contained all eight domains that define the hallmarks of these types of genes (FIG. 18).

Hydrophobicity analysis (PepPlot, Genetics Computer Group) of the Gene 216 amino acid sequence revealed the presence of two hydrophobic regions (FIG. 20). One region is located at the amino terminus of the protein and is the putative the signal sequence. The other hydrophobic region is located near the carboxyl terminus and is the putative transmembrane domain that anchors the protein to the cell surface. Computational biology analysis (http://blocks.fhcrc.org) of the Gene 216 cytoplasmic domain revealed the presence of a putative SH2 and SH3 binding domain as well as a putative casein kinase I phosphorylation site (FIG. 19). These sites may contribute to a role in bi-directional signaling, a function attributed to ADAM proteins.

Sequence analyses indicated that Gene 216 is a novel member of the ADAM family. Gene 216 is most closely related to ADAMs 8, 9, 12,15, and 19, a branch of the family that is known to possess an active metalloprotease domain. Table 6 lists the 5 most similar BLASTP hits using the Gene 216 amino acid sequence as a query. Based on BLASTN and BLASTP analysis, Gene 216 nucleotide sequence shares the 37% identity with the ADAM 19 nucleotide sequence; and Gene 216 amino acid sequence shares 58% identity with the ADAM 19 amino acid sequence.

TABLE 6
Top 5 Hits from BLAST Analysis of Gene 216 protein

| Hit | GenBank | Locus Description | Smallest Sum |
|---|---|---|---|
| 1 | U66003 | Xenopus laevis (ADAM 13) | 5.5e−166 |
| 2 | AF019887 | Mus musculus metalloprotease- | 1.2e−139 |
| | | disintegrin meltrin beta | |
| 3 | AF134707 | Homo sapiens disintegrin and | 1.6e−139 |
| | | metalloprotease domain 19 | |
| | | (ADAM19) | |
| 4 | S60257 | Mouse mRNA for meltrin alpha | 1.8e−121 |
| 5 | AF023476 | Homo sapiens meltrin-L | 4.9e−119 |
| | | precursor (ADAM12) | |

Table 7 lists the top two hits from BLIMPS analysis of the Block protein motif database (http://blocks.fhcrc.org/).

TABLE 7
Top 2 Hits from BLIMPS Analysis of Gene 216 protein

| Description | Strength Score | AA# | AA | Sequence | |
|---|---|---|---|---|---|
| Disintegrins proteins | 1950 | 1597 | 377 | CCfAhnCsLRPGAQCAh- | (SEQ ID NO:335) |
| | | | | GdCCvRCIIKpAGaI- | |
| | | | | CRqAMGDCDIPEfCT- | |
| | | | | GTSshCPP | |
| Zinc metallopeptidases | 1173 | 1276 | 276 | TMAHEIGHSLG | (SEQ ID NO:336) |

2. Amino Acid Changes:

In total, there were 9 SNPs within the open reading frame of Gene 216. See Example 10 for details on polymorphism identification and FIG. 19 for resulting changes to the protein sequence. Seven of the nine SNPs constituted an amino acid change and the other 2 were synonymous. Of the 7 amino acid changes, 4 were clustered toward the carboxyl terminus of the protein: one within the identified transmembrane domain and 3 within the identified cytoplasmic domain.

One SNP located in an identified SH2 binding domain resulted in a significant amino acid change: methionine (hydrophobic) to threonine (polar). The remaining two SNPs in the identified cytoplasmic domain resulted in significant amino acid changes: proline (hydrophobic) to serine (polar) and glutamine (polar) to histidine (basic). These amino acid changes may disturb the signaling properties of the Gene 216 protein. In addition, the valine to isoleucine amino acid change in the putative transmembrane domain may affect signaling efficiency.

The two SNPs in the identified pro-domain generated significant amino acid changes: tyrosine (polar) to histidine (basic) and threonine (polar) to alanine (hydrophobic). Since the ADAM pro-domain is cleaved during activation of the catalytic domain, it is possible that these amino acid changes affect the cleavage process. One SNP in the identified catalytic domain resulted in a change from alanine (hydrophobic) to valine (hydrophobic). This amino acid change may affect sheddase efficiency.

Notably, amino acid changes in the identified Gene 216 catalytic domain, especially within the metalloprotease domain, would be of great interest, as this domain is critical to sheddase function. Recently, the X-ray crystallographic data of the snake venom catalytic domain was determined and deposited in the public domain (http://www.rcsb.orgpdb/cgi/explore.cgi? pid=9267984771616 & pdbld=1 C9G; Accession No. 1 C9GA). This information can be utilized to determine whether an amino acid change alters the folding of the catalytic domain of the Gene 216 protein. In particular, the sequence of the catalytic domain of Gene 216 protein can be plotted as X-ray crystallographic coordinates and used to determine changes in the tertiary structure of this domain.

3. Biological Role of Gene 216:

ADAMs are part of a very large superfamily called zinc-dependent metalloproteases (Stone et. al., 1999, J. Prot. Chem. 18:447465). Gene 216 represents a novel member of the ADAM family that is closely related to ADAM 19, a gene that was found to participate in the proteolytic processing of the membrane anchored protein neuregulin 1 (NRG1) (Shirakabe et. al., 2001, J. Biol. Chem. 276(12):9352-8). The expression and activation of ADAM 19 protein has been localized to the trans-Golgi apparatus. This has been observed for other ADAM proteins (Lum et al., 1998, J. Biol. Chem. 273:26236-26247; Roghani et. al., 1999, J. Biol. Chem. 274:3531-3540; Shirakabe et. al., 2001, J. Biol. Chem. 276(12):9352-8). These data suggest that the ADAM genes, and Gene 216, encode proteins that function in the trans-Golgi apparatus as intracellular processing enzymes. The processed substrates of these enzymes may be released into the cytosol as part of a signal transduction cascade leading to the cell surface.

The substrate of ADAM 19, NRG1, belongs to a group of growth factors (neuregulins) that are members of the epidermal growth factor family. The neuregulins participate in an array of biological effects that are mediated by the epidermal growth factor family of tyrosine kinase receptors. Data suggest that the proteolytically cleaved isoform of NRG1, NRG-β1, may induce the tyrosine phosphorylation of EGFR2 and EGFR3 in differentiated muscle cells (Shirakabe et. al., 2001, J. Biol. Chem. 276(12):9352-8). The sequence similarity of Gene 216 protein and ADAM 19 protein suggests that the neuregulins or their isoforms serve as substrates for Gene 216 protein. The Gene 216-processed neuregulins or isoforms may then serve as ligands for EGFR1.

Epidermal growth factor receptor (EGFR1) plays a pivotal role in the maintenance and repair of epithelial tissue. Following injury in bronchial epithelium, EGFR1 is upregulated in response to ligands acting on it or through transactivation of the EGFR1 receptor. This results in the increased proliferation of cells and airway remodeling at the point of insult, leading to the repair of the bronchial epithelium (Polosa et. al., 1999, Am. J. Respir. Cell Mol. Biol. 20:914-923; Holgate et. al., 1999, Clin. Exp. Allergy Suppl 2:90-95).

In asthma, the bronchial epithelium is highly abnormal, with structural changes involving separation of columnar cells from their basal attachments and functional changes that include increased expression and release of proinflammatory cytokines, growth factors, and mediator-generating enzymes. Beneath this damaged structure are the subepithelial myofibroblasts that have been activated to proliferate. This, in turn, causes excessive matrix deposition leading to abnormal thickening and increased density of the subepithelial basement membrane.

Immunocytochemical studies have shown that both TGF-β and EGFR1 are highly expressed at the area of injury and that parallel pathways could be operating in the repairing epithelial cells (Puddicombe et. al., 2000, FASEB J. 14:1362-1374). EGFR1 stimulates epithelial repair and TGF-β regulates the production of profibrogenic growth factors and proinflammatory cytokines leading to extracellular matrix synthesis. As EGFR1 is involved in regulating a number of different stages of epithelial repair (survival, migration, proliferation and differentiation), any inhibitory effects that act on the receptor may cause the epithelium to be held in a “state of repair” (Holgate et. al., 1999, Clin. Exp. Allergy Suppl 2:90-95).

Without wishing to be bound by theory, it is possible that a variant Gene 216 protein induces the epithelium into a continuous “state of repair” by functioning improperly and failing to release its substrate (a member of the neuregulin family) that serves as the ligand for EGFR1. This, in turn, may cause the observed increase in EGFR1 expression. Under these circumstances, the TGF-β pathway remains active, producing a continuous source of proinflammatory products as well as growth factors that drive airway wall remodeling causing bronchial hyperresponsiveness, a phenotype of asthma.

It is also possible that the disintegrin-like domain of Gene 216 plays a role in respiratory diseases. Integrins are a family of heterodimeric transmembrane receptors that mediate cell-cell and cell-extracellular matrix interaction (Hynes, 1992, Cell 69:11). Integrins mediate angiogenesis (Brooks et al., 1994, Science 264:569), which plays a major role in various pathological mechanisms, such as tumor growth, metastasis, diabetic retinopathy, and certain inflammation diseases (Folkman, 1995, N. Engl. J. Med. 333:1757). Disintegrins act as integrin ligands that disrupt cell-matrix interactions (C. P. Blobel and J. M. White, 1992, Curr. Opin. Cell Biol. 4:760-5) and inhibit angiogenesis (C. H. Yeh et al., 1998, Blood 92:3268-3276). Without wishing to be bound by theory, it is possible that the disintegrin-like domain of the Gene 216 polypeptide inhibits angiogenesis in the respiratory system. Gene 216 variants that have partly functional or non-functional disintegrin activity may lack anti-angiogenesis function. These Gene 216 variants may give rise to angiogenesis and inflammation in the respiratory system, a phenotype of asthma.

### EXAMPLE 9

### Identification of the Mouse Homolog for Gene 216

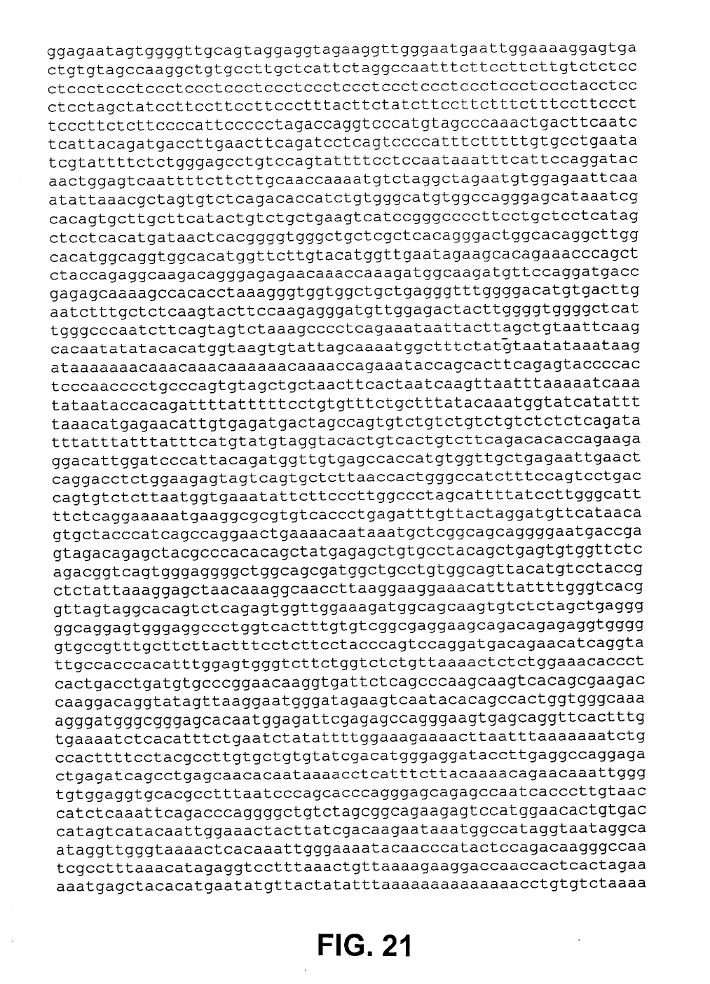

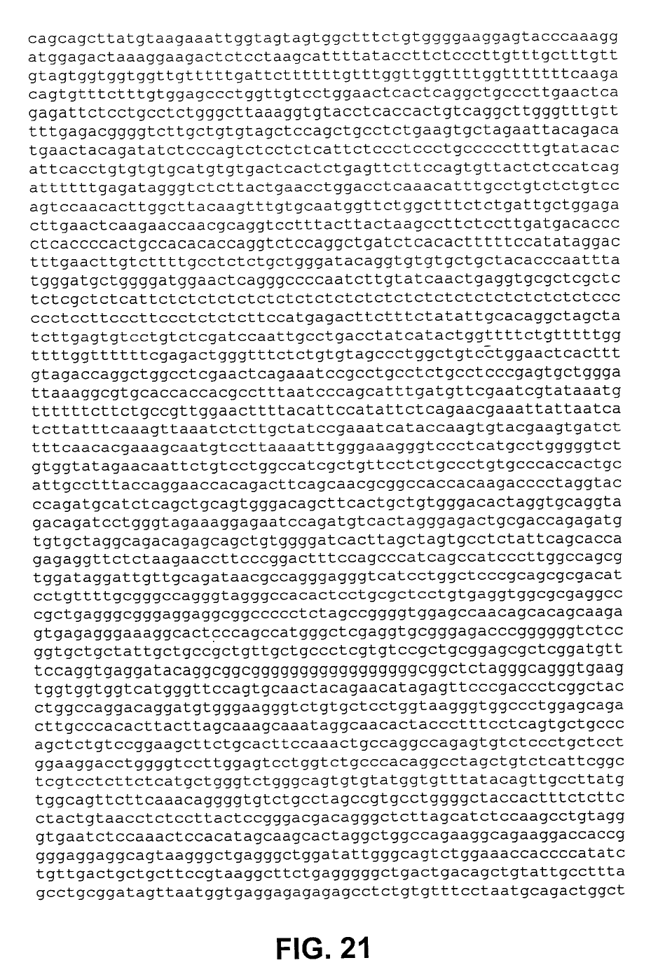

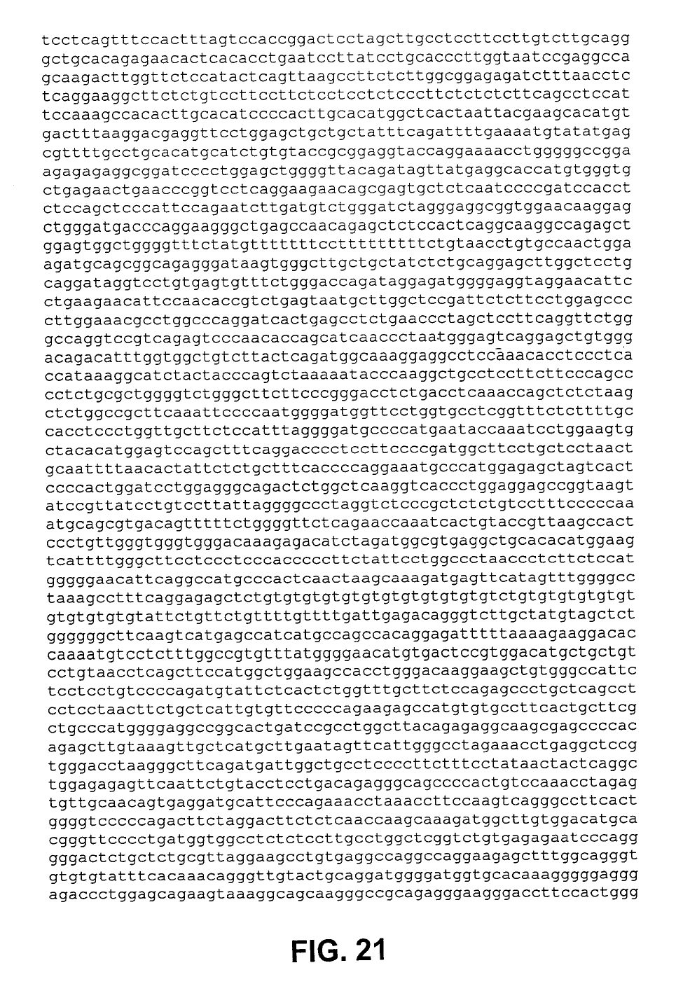

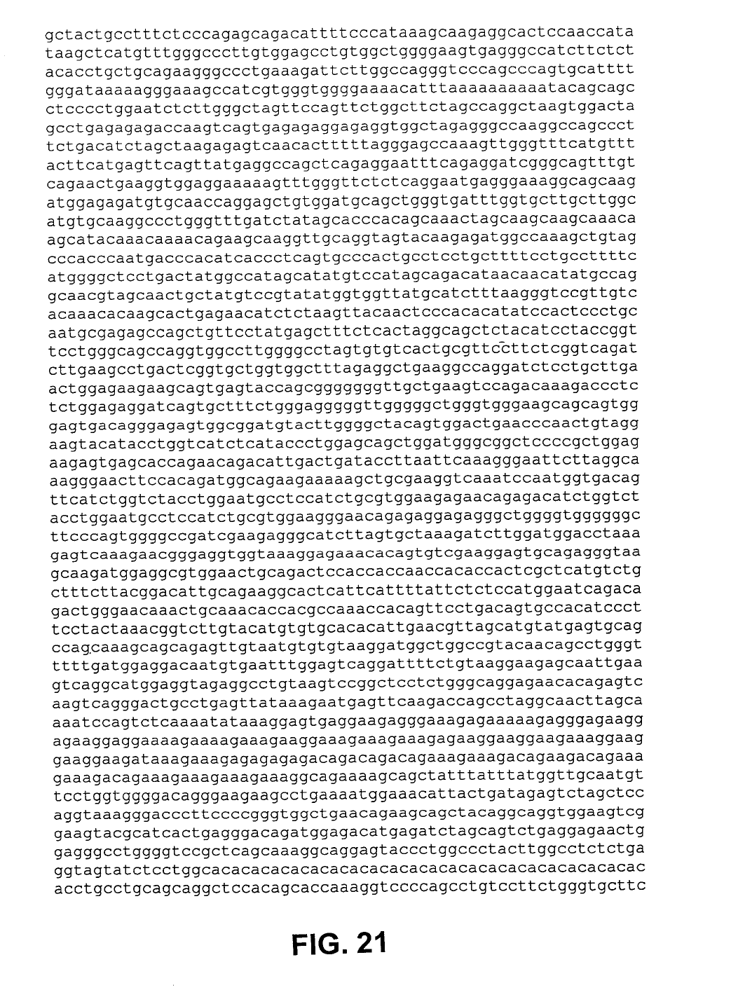

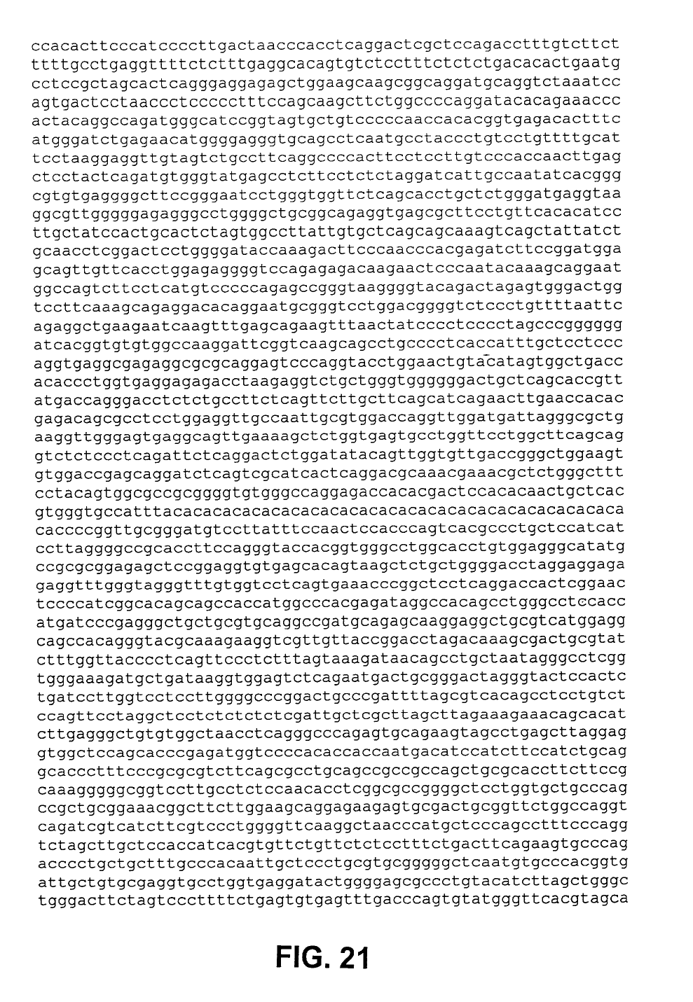

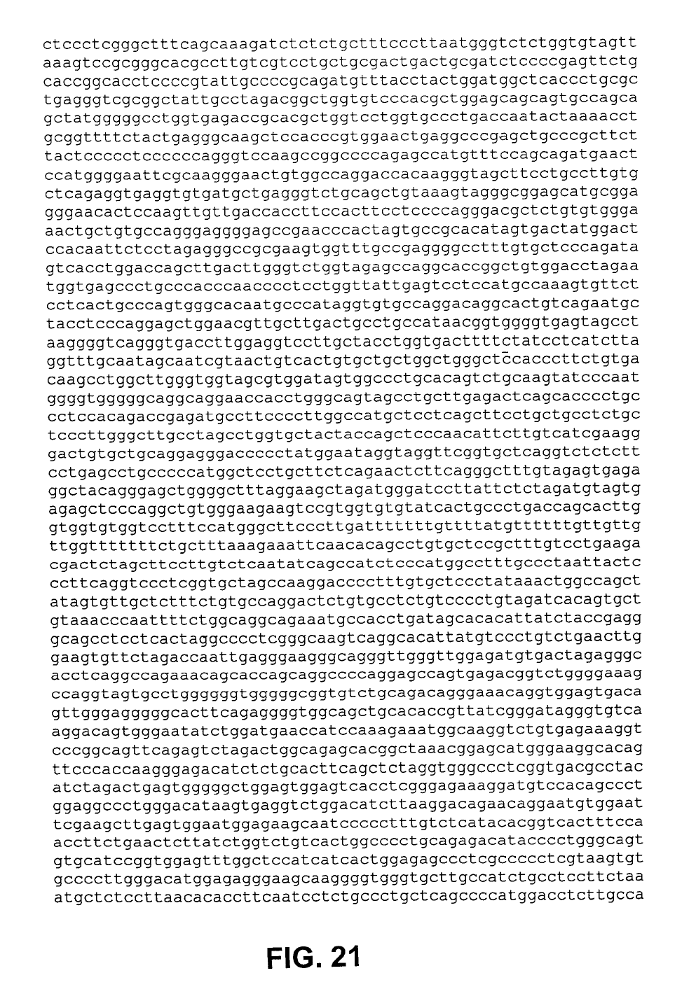

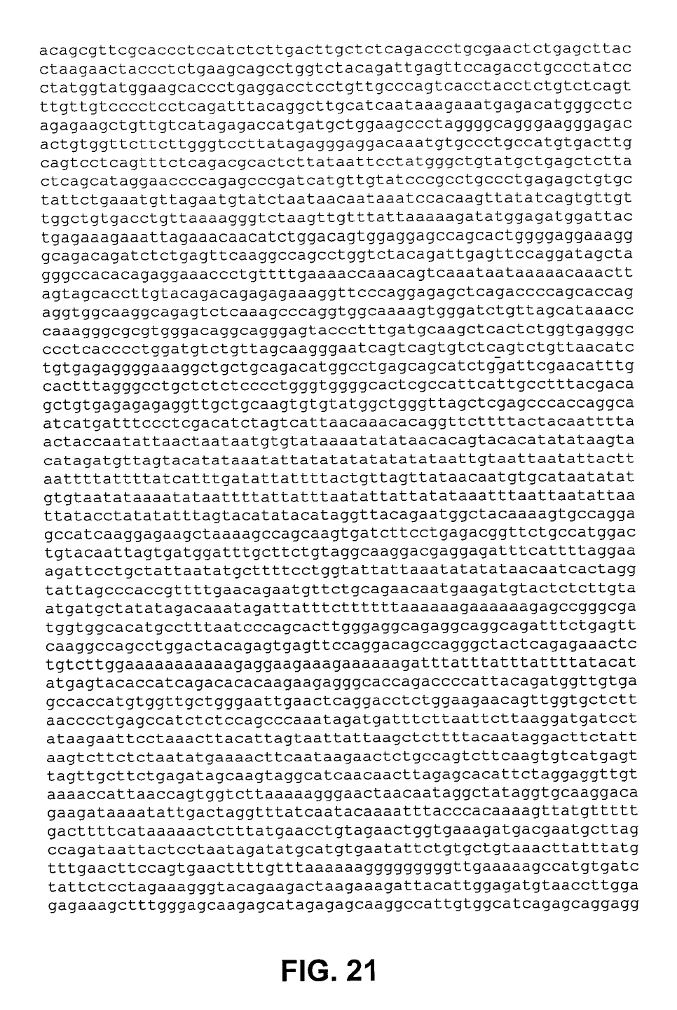

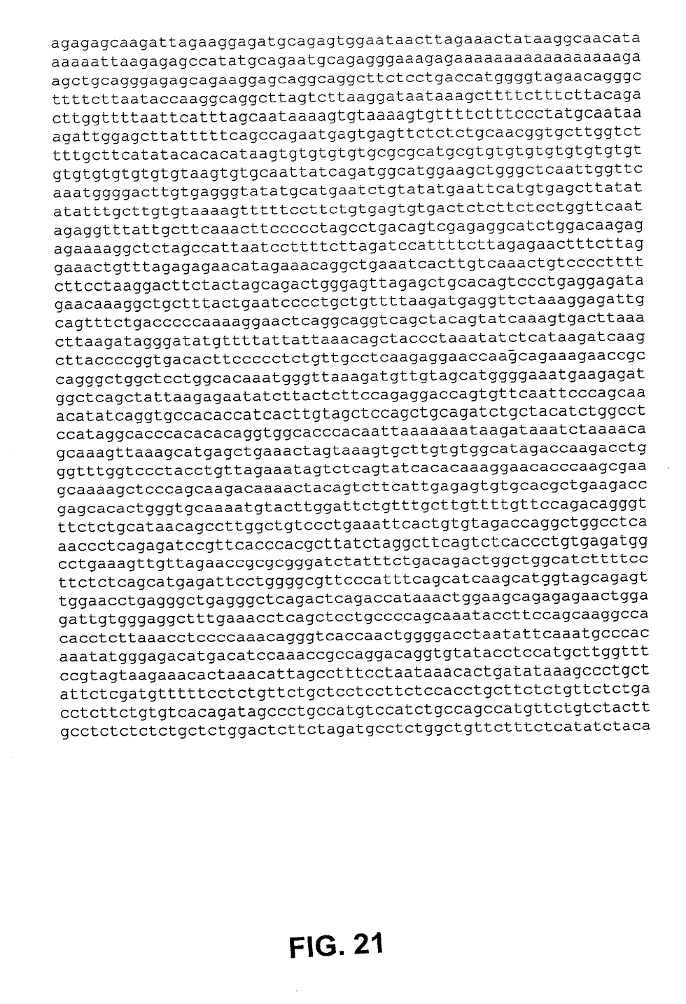

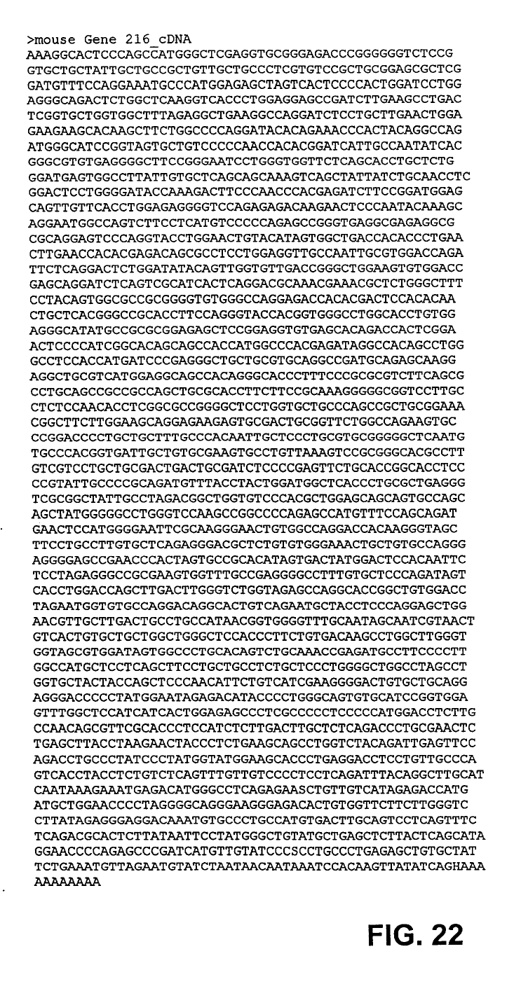

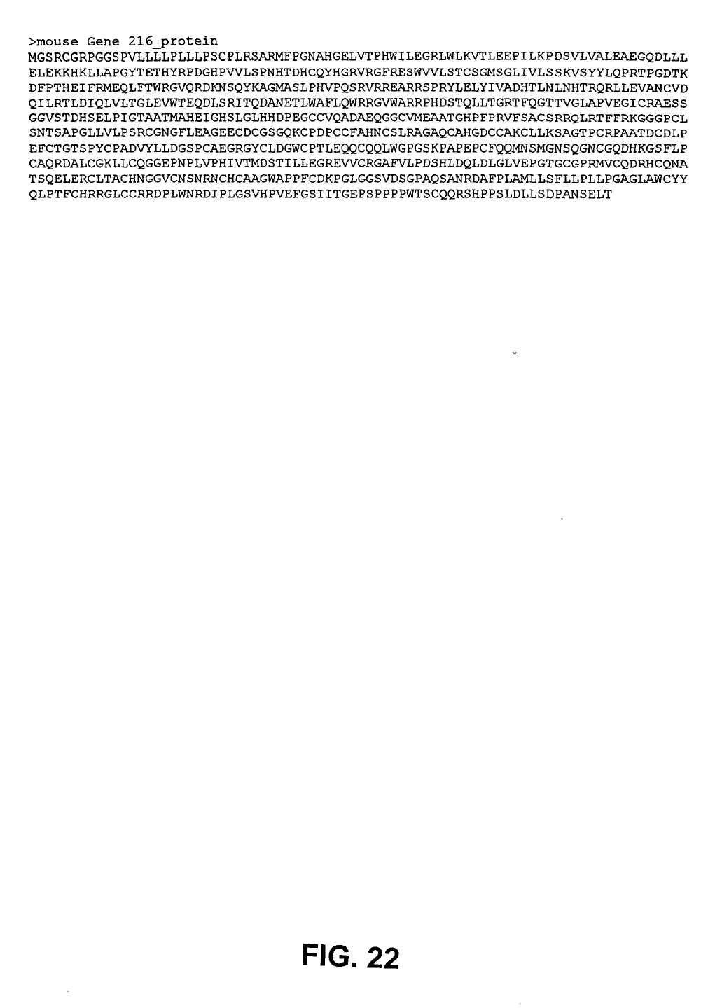

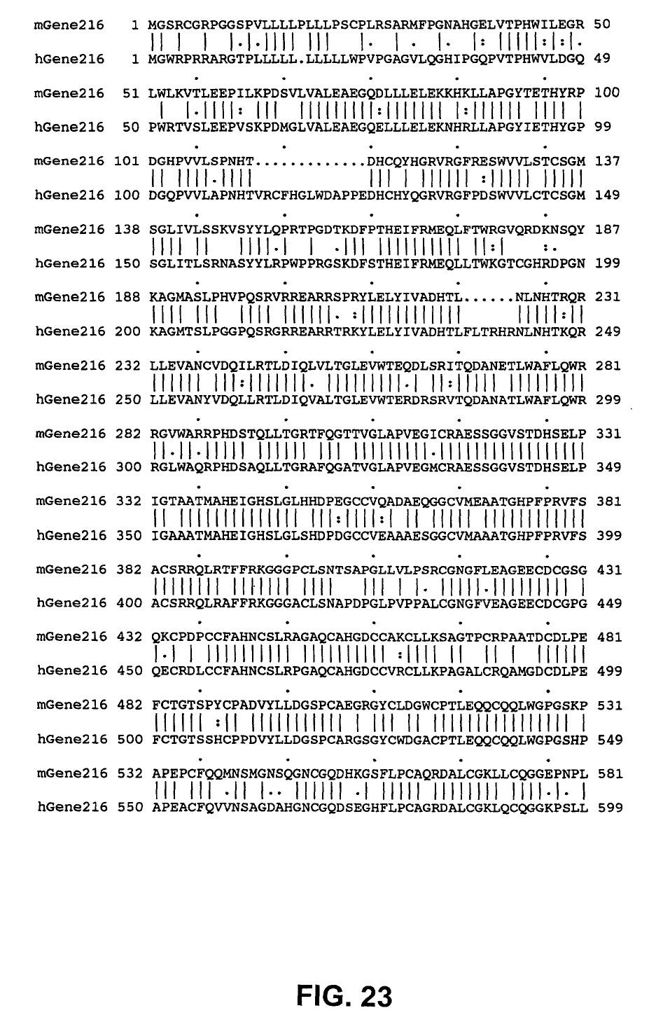

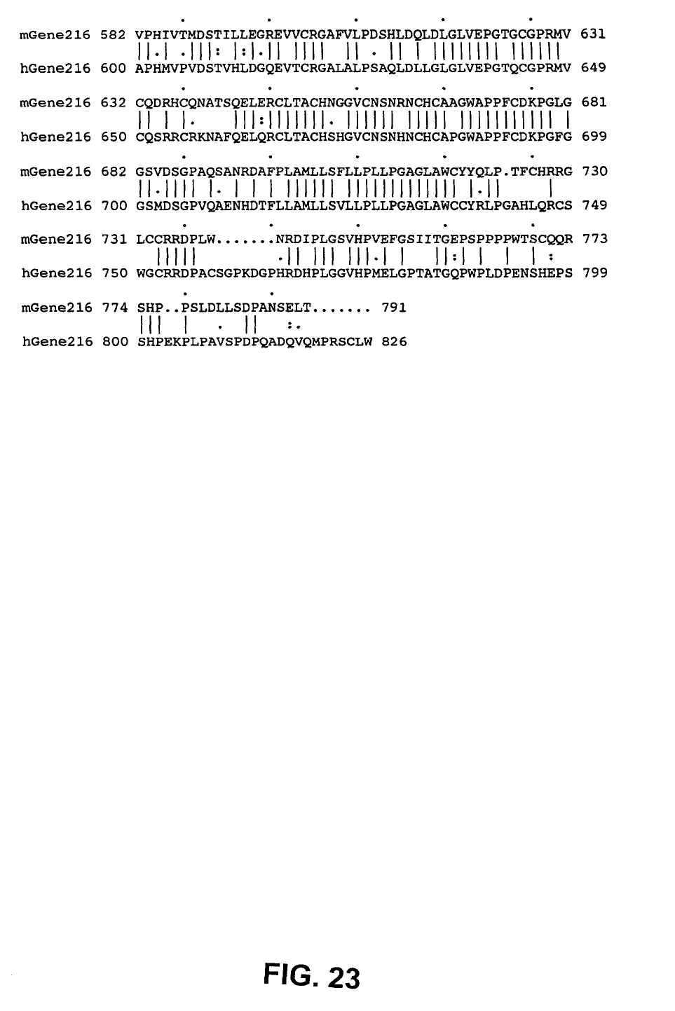

The mouse ortholog of Gene 216 was identified by TBLASTN analysis of Gene 216 against mouse dbEST. BLAST analysis identified three mouse ESTs that were partially homologous to the human sequence but were not 100% homologous to any known mouse ADAM genes. The three mouse ESTs were 100% identical to a partially sequenced mouse BAC (BAC389B9; Accession Number AF155960). This BAC maps to mouse chromosome 2 in a region that is syntenic to human chromosome 20p13. The 47 kb BAC sequence was analyzed for potential genes using the Genscan gene prediction program (Burge and Karlin, J. Mol. Biol., 268:78-94). Additional putative exons were identified based on comparison of the human Gene 216 protein to the mouse BAC by TBLASTN. The results identified a mouse gene that contained an ORF of 2124 bp encoding a protein of 707 amino acids. The genomic nucleotide sequence of the mouse homolog is depicted in FIG. 21 and the corresponding amino acid sequence is depicted in FIG. 22. The mouse amino acid sequence was analyzed by BLASTP analysis and found to have homology to mouse and human ADAM proteins. The mouse amino acid sequence was aligned against the amino acid sequence of human Gene 216 (BestFit, http://www.gcg.com) (FIG. 23). The results showed that the mouse and human proteins shared ˜70% identity at the amino acid level. This indicated that the mouse sequence was the murine ortholog of human Gene 216.

### EXAMPLE 10

### Polymorphism Identification

Polymorphisms were identified in the chromosome 20 region and subsequently used in association studies. Most of the data focused on the region of Gene 216.

1. Single Nucleotide Polymorphism (SNP) Discovery:

An efficient tiered approach was used for mutation analysis. First, PCR assays were developed across exons to include the consensus splice sites. Assays were designed for all exons that contribute to the open reading frame of the gene. This strategy ensured the detection of mutations that would result in the modification of the protein sequence as well as mutations that would be predicted to disrupt mRNA splicing. The identified promoter and putative regulatory element for Gene 216 and a large intronic region were assayed for polymorphisms as well. Second, a total of 77 individuals were tested for polymorphisms using fluorescent SSCP (single strand conformational polymorphism). This sample size provided a 99% power to detect a polymorphism with a frequency of 3% or greater. Briefly, PCR was used to generate templates from asthmatic individuals that showed increased sharing for the 20p13-p12 chromosomal region and contributed towards linkage. Non-asthmatic individuals were used as controls. Enzymatic amplification of Gene 216 was accomplished using PCR with oligonucleotides flanking each exon as well as the putative 5′ region. Primers were chosen to amplify each exon as well as 15 or more base pairs within each intron on either side of the splice site. The forward and the reverse priers were labeled with two different dye colors to allow analysis of each strand and confirm variants independently. Standard PCR assays were utilized for each exon primer pair following optimization. Buffer and cycling conditions were specific to each primer set. The products were denatured using a formamide dye and electrophoresed on non-denaturing acrylamide gels with varying concentrations of glycerol (at least two different glycerol concentrations).

Primers utilized in fluorescent SSCP experiments to screen coding and non-coding regions of Gene 216 for polymorphisms are provided in Table 8. Column 1 lists the genes targeted for mutation analysis. Column 2 lists the specific exons analyzed. Column 3 lists the primer names. Columns 4 and 5 list the forward primer sequences and corresponding SEQ ID NOS, respectively. Columns 5 and 6 list the reverse primer sequences and corresponding SEQ ID NOS, respectively

TABLE 8

| | | | | SEQ ID | | SEQ ID |
|---|---|---|---|---|---|---|
| Gene | Exon | Assay Name | Primer Sequence | NO | Primer Sequence | NO: |
| 216 | 216_A | 502_216_A_F_503_216_A_R | Ctgcctagaggccgagga | 63 | agctctgagcagaacccatc | 106 |
| 216 | 216_A | 1623_216 A_F_1624_216_A_R | Caggagaccacggaagatcg | 64 | ctcgagggggtggagctg | 107 |
| 216 | 216_A | 1625_216_A_F_1626_216_A_R | Ttgcctgaaccttcctatcc | 65 | gagaggaggagagaaccgct | 108 |
| 216 | 216_B | 293_216_B_F_294_216_B_R | Cccctgtgttcctcaggtc | 66 | agtgacttggtggttctggg | 109 |
| 216 | 216_C | 295_216_C_F_296_216_C_R | Gctccacactctttcttgcc | 67 | tgtcatctgcaccctctctg | 110 |
| 216 | 216_D | 297_216_D_F_298_216_D_R | Aggcaggaggaagctgaat | 68 | aagagggagggtgtggtagg | 111 |
| 216 | 216_E | 1290_216_E_F_1291_216_E_R | Cctaccacaccctccctctt | 69 | gtgatcaggccactagggtg | 112 |
| 216 | 216_F | 299_216_F_F_300_216_F_R | Cctacccctctgcacccta | 70 | atacagcattcccaetccca | 113 |
| 216 | 216_G | 301_216_G_F_302_216_G_R | aacttccttctgggagctgg | 71 | gaaggcagaaatcccggt | 114 |
| 216 | 216_H | 700_216_H_F_701_216_H_R | cacaccctggtgaggagaga | 72 | caccagcacctgcctgtc | 115 |
| 216 | 216_I | 305_216_I_F_306_216_I_R | ccacgaaggaccaccg | 73 | gggtcagaggcacccac | 116 |
| 216 | 216_J | 889_216_J_F_890_216_J_R | ctcacgtgggtgcctctg | 74 | gccgtagagcctcctgtct | 117 |
| 216 | 216_K | 891_216_K_F_892_216_K_R | ctctacggccgcagtgac | 75 | gacgaccaaagaaacgcag | 118 |
| 216 | 216_L | 311_216_L_F_312_216_L_R | gtccctccatgcccaatg | 76 | tgagcggagagggcaagt | 119 |
| 216 | 216_L | 313_216_L_F_314_216_L_R | caggttaagtcggctcgc | 77 | aaaccctcaccctgaacctt | 120 |
| 216 | 216_M | 315_216_M_F_316_216_M_R | ctctctctgccttccccac | 78 | aagggtgctcgtgtcctct | 121 |
| 216 | 216_N | 317_216_N_F_318_216_N_R | tctactgtggggaagatggg | 79 | ccactcagctccactcccta | 122 |
| 216 | 216_O | 319_216_O_F_320_216_O_R | cccctctacttcctcccca | 80 | ggattcaaacggcaaggag | 123 |
| 216 | 216_P | 321_216_P_F_322_216_P_R | gaccttggggttcctaatcc | 81 | gctgagtcctgagcaggtg | 124 |
| 216 | 216_Q | 323_216_Q_F_504_216_Q_R | gtgcacctgctcaggactc | 82 | gaaccgcaggagtaggctc | 125 |
| 216 | 216_R | 325_216_R_F_326_216_R_R | cctggactcttatcacgttgc | 83 | atatggtcagcaggagaccc | 126 |
| 216 | 216_S | 327_216_S_F_328_216_S_R | ttaccctccaccatttctcc | 84 | gcatcctggtctccatgataa | 127 |
| 216 | 216_S | 1308_216_S_F_1309_216_S_R | gtggagagggaagggagaag | 85 | gaggctttgaatccaggtcc | 128 |
| 216 | 216_T | 1294_216_T_F_1295_216_T_R | ccccatgggttgaatttaca | 86 | cagcaagacaccgcatctac | 129 |
| 216 | 216_T | 1296_216_T_F_1297_216_T_R | gcagctaggcctacaggtaca | 87 | gggacagagggaaccattta | 130 |
| 216 | 216_T | 1298_216_T_F_1299_216_T_R | accacgcctatagccaacat | 88 | ttccttcctgtttcttccca | 131 |
| 216 | 216_T | 1300_216_T_F_1301_216_T_R | aggtgtagcactgggattgg | 89 | gtcctgggagtctggtgtgt | 132 |
| 216 | 216_T | 1302_216_T_F_1303_216_T_R | ccccaggaccactagcttct | 90 | aggaacccagagccacacta | 133 |
| 216 | 216_T | 1304_216_T_F_1305_216_T_R | attgagctggagagtgtgcc | 91 | tgcctctggtgagaggtagc | 134 |
| 216 | 216_T | 1306_216_T_F_1307_216_T_R | ttcaagttcctggagtggct | 92 | ttcctggatcactggtcctc | 135 |
| 216 | 216_AA | 1619_216_AA_F_1620_216_AA_R | acaaggaccctctaaacgca | 93 | ttcgagcagtgagagaaacct | 136 |
| 216 | 216_PQ | 1465_216_PQ_F_1466_216_PQ_R | acccttctgtgacaagccag | 94 | ctgggagtcggtagcaaca | 137 |
| 216 | 216_QR | 1467_216_QR_F_1468_216_QR_R | gtgttgctaccgactcccag | 95 | aggccactggaacctcct | 138 |
| 216 | 216_QR | 1469_216_QR_F_1470_216_QR_R | cccaggtgcagagagcag | 96 | gcagcatggtacagggactg | 139 |
| 216 | 216_QR | 1471_216_QR_F_1472_216_QR_R | gctcctcttgtccactctcct | 97 | cagctgaccagtggtatgga | 140 |
| 216 | 216_QR | 1473_216_QR_F_1474_216_QR_R | gccacttcctctgcacaaat | 98 | tgtcagacatggccacagag | 141 |
| 216 | 216_QR | 1475_216_QR_F_1476_216_QR_R | ttctctgtgacctgggtggt | 99 | agggtcctcttagctgccac | 142 |
| 216 | 216_QR | 1477_216_QR_F_1478_216_QR_R | atttgggccagagatggg | 100 | aggccttgtcatttcctgtg | 143 |
| 216 | 216_QR | 1479_216_QR_F_1480_216_QR_R | ggcagaggagcaaggtgg | 101 | caaagaaccttggatgtccg | 144 |
| 216 | 216_QR | 1481_216_QR_F_1482_216_QR_R | atggcttggaatcatcaagg | 102 | ctcagctcccttcctgctc | 145 |
| 216 | 216_QR | 1483_216_QR_F_1484_216_QR_R | tagagagaggaggtgccagc | 103 | ctgtgtgggccatctttg | 146 |
| 216 | 216_RS | 1485_216_RS_F_1486_216_RS_R | aaagatggcccacacagg | 104 | ggagaaatggtggagggtaa | 147 |
| 216 | 216_ST | 1487_216_ST_F_1488_216_ST_R | agaactctcatgagcccagc | 105 | aaagccacagcttctccct | 148 |
| 216 | 216_ST | 1489_216_ST_F_1490_216_ST_R | aggtttctgggctcaggtta | 149 | caggatcttggcatctggac | 153 |
| 216 | 216_UP | 1463_216_UP_F_1464_216_UP_R | gtaggtgtgccagagcagg | 150 | ctggcttgtcacagaagggt | 154 |
| 216 | 216_U | 1292_216_U_F_1293_216_U_R | tgtggacctagaatggtgagc | 151 | ctggagcacagtggcagtta | 155 |
| 216 | 216_V | 1736_216_V_F_1737_216_V_R | caaagtcacacaacaagcgg | 152 | tttggtcgtccctcagtttc | 156 |

Once polymorphisms were identified, multiple individuals representative of each SSCP pattern and two genomic controls were sequenced for polymorphism validation and to identify SNPs. The variants detected in the initial set of asthmatic and normal individuals were subject to fluorescent sequencing (ABI) using a standard protocol described by the manufacturer (Perkin Elmer). In cases where SSCP did not identify polymorphisms in Gene 216, sequence information was obtained from 16 individuals that were identical by descent (IBD) in the region, and from 4 controls to ensure that potential polymorphisms were identified.

Primers utilized in DNA sequencing for purposes of confirming polymorphisms detected using fluorescent SSCP are provided in Table 9. Column 1 lists the specific exons sequenced. Column 2 lists the forward primer names, column 3 lists the forward primer sequences, and column 4 lists the corresponding SEQ ID NOS. Column 5 lists the reverse primer names, column 6 lists the reverse primer sequences, and column 7 lists the corresponding SEQ ID NOS.

TABLE 9

| | | | SEQ ID | | | SEQ |
|---|---|---|---|---|---|---|
| Exon | Forward | Forward Seq | NO: | Reverse Name | Reverse Seq | ID NO |
| 216_A | MDSeg_101_216_A_F | cctctcaggagtagaggccc | 157 | MDSeq_101_216_A_R | ccaagcacacttgagcgtc | 177 |
| 216_A | MDSeq_175_216_A_F | agcggttctctcctcctctc | 158 | MDSeq_175_216_A_R | agccatgccctctgcttt | 178 |
| 216_A | MDSeq_213_216_A_F | cctctcaggatagaggccc | 159 | MDSeq_213_216_A_R | cagcccaagcacacttga | 179 |
| 216_A | MDSeq_334_216_A_F | atgttactgaggccgaaagg | 160 | MDSeq_334_216_A_R | cccatagctgtgagctcctc | 180 |
| 216_B | MDSeq_296_216_R_F | ccctttccagccttctcttt | 161 | MDSeq_296_216_B_R | aaagcttcaggacccacaaa | 181 |
| 216_C | MDSeq_297_216_C_F | caggactgcaaacatcctga | 162 | MDSeq_297_216_C_R | atcttggtccctgccattc | 182 |
| 216_D | MDSeq_61_216_D_F | tccctggtgcttcccata | 163 | MDSeq_61_216_D_R | gagggagctctttcccca | 183 |
| 216_E | MDSeq_245_216_E_F | aggcaggaggaagctgaat | 164 | MDSeq_245_216_E_R | ggaccaccaggaaggctg | 184 |
| 216_F | MDSeq_57_216_F_F | cctcttgcccctcttgct | 165 | MDSeq_57_216_F_R | aaccccagctcccagaag | 185 |
| 216_G | MDSeq_336_216__G_F | cctgaatgtccagagtcctga | 166 | MDSeq_336_216_G_R | ctgctcacctggaaaggaac | 186 |
| 216_H | MDSeq_155_216_H_F | ggcctcgagtcccagtattt | 167 | MDSeq_155_216_H_R | actgcaggaaggcccagag | 187 |
| 216_I | MDSeq_363_216_I_F | agagcctcctgtctctccct | 168 | MDSeq_363_216_I_R | accgaaacttgaaccacacc | 188 |
| 216_J | MDSeq_181_216_J_F | tcgccctcagcttctcag | 169 | MDSeq_181_216_J_R | tgagggacgaccaaagaaac | 189 |
| 216_K | MDSeq_182_216_K_F | tcacgtgggtgcctctga | 170 | MDSeq_182_216_K_R | caaagtcacacaacaagcgg | 190 |
| 216_L | MDSeq_106_216_L_F | gggttacttcccctctctgg | 171 | MDSeq_106_216_L_R | gaacctgagggcaccaatta | 191 |
| 216_M | MDSeq_337_216_M_F | ctgggctttccaccctgg | 172 | MDSeq_337_216_M_R | ttggccttagttaattggtgc | 192 |
| 216_N | MDSeq_338_216_N_F | ctgggctttccaccctgg | 173 | MDSeq_338_216_N_R | ttggccttagttaattggtgc | 193 |
| 216_O | MDSeq_49_216_O_F | tccaggtggtgaactctgc | 174 | MDSeq_49_216_O_R | ctggagcacagtggcagtta | 194 |
| 216_P | MDSeq_248_216_P_F | tagaatggtgagctctgccc | 175 | MDSeq_248_216_P_R | aggagtaggctcaggaagca | 195 |
| 216_Q | MDSeq_96_216_Q_F | gaccttggggttcctaatcc | 176 | MDSeq_96_216_Q_R | tgtactgggaggtagagggc | 196 |
| 216_R | MDSeq_50_216_R_F | agagggtgacttggagcaga | 197 | MDSeq_50_216_R_R | ccagaaacctgattaggggg | 219 |
| 216_S | MDSeq_262_216_S_F | aggcaataacccactcagga | 198 | MDSeq_262_216_S_R | tacctctcaccagaggcagg | 220 |
| 216_T | MDSeq_255_216_T_F | cccatgggttgaatttacata | 199 | MDSeq_255_216_T_R | gccagaagctagtggtcctg | 221 |
| 216_T | MDSeq_256_216_T_F | gcctctggtgatcctcctac | 200 | MDSeq_256_216_T_R | gcaggcagcttggaagttt | 222 |
| 216_T | MDSeq_257_216_T_F | actcagtcgaaccatagggc | 201 | MDSeq_257_216_T_R | ttatcatggagaccaggatgc | 223 |
| 216_T | MDSeq_258_216_T_F | tgtgtgacctttgcttctgg | 202 | MDSeq_258_216_T_R | gacctggattcaaagcctcc | 224 |
| 216_T | MDSeq_358_216_T_F | gcatgaagcaatgggagaat | 203 | MDSeq_358_216_T_R | atgttggctataggcgtggt | 225 |
| 216_T | MDSeq_365_216_T_F | actcagtcgaaccatagggc | 204 | MDSeq_365_216_T_R | ttatcatggagaccaggatgc | 226 |
| 216_U | MDSeq_244_216_U_F | gcaggaaggtgtcatggtct | 205 | MDSeq_244_216_U_R | ctgagtggagggagcagaag | 227 |
| 216_U | MDSeq_292_216_U_F | gcaggaaggtgtcatggtct | 206 | MDSeq_292_216_U_R | ctgagtggagggagcagaag | 228 |
| 216_V | MDSeq_389_216_V_F | gggcattggagaggcaag | 207 | MDSeq_389_216_V_R | ccatgagatcggccacag | 229 |
| 216_AA | MDSeq_360_216_AA_F | tctgcctcccagattcaagt | 208 | MDSeq_360_216_AA_R | atttcaaggctgcaatgagg | 230 |
| 216_PQ | MDSeq_300_216_PQ_F | agaatgccttccaggagctt | 209 | MDSeq_300_216_FQ_R | acttctttccatggcctctg | 231 |
| 216_QR | MDSeq_301_216_QR_F | gtgttgctaccgactcccag | 210 | MDSeq_301_216_QR_R | accacccaggtcacagagaa | 232 |
| 216_QR | MDSeq_303_216_QR_F | ctgcttcctgagcctactcc | 211 | MDSeq_303_216_QR_R | tcccaagaccaggctatgtc | 233 |
| 216_QR | MDSeq_321_216_QR_F | aacaggaggttccagtggc | 212 | MDSeq_321_216_QR_R | ctggggatgagaagcagc | 234 |
| 216_QR | MDSeq_322_216_QR_F | agcgagttgtgattgagggt | 213 | MDSeq_322_216_QR_R | cttctcccttccctctccac | 235 |
| 216_QR | MDSeq_361_216_QR_F | tgtgcaggctgaaagtatgc | 214 | MDSeq_361_216_QR_R | atttgtgcagaggaagtggc | 236 |
| 216_QR | MDSeq_362_216_QR_F | gccacttcctctgcacaaat | 215 | MDSeq_362_216_QR_R | catttcctccaggctctgac | 237 |
| 216_RS | MDSeq_339_216_RS_F | ctgagcccagaaacctgatt | 216 | MDSeq_339_216_RS_R | tcagagcctggaggaaatgt | 238 |
| 216_ST | MDSeq_302_216_ST_F | gtgagtgaggcaccaggg | 217 | MDSeq_302_216_ST_R | gttcctggagtgggtgggt | 239 |
| 216_UP | MDSeq_359_216_UP_F | cctagatggccaggaagtga | 218 | MDSeq_359_216_UP_R | ctgggagtcggtagcaaca | 240 |

Single nucleotide polymorphisms (SNPs) that were identified in Gene 216 are provided in Table 10. Column 1 lists the SNP numbers (1-48). Column 2 lists the exons that either contain the SNPs or are flanked by intronic sequences that contain the SNPs. Column 3 lists the PMP sites for the SNPs. A “−” denotes polymorphisms which are 5′ of the exon that are within the intronic region. The corresponding number is given from the 3′ to 5′ direction. A “+” denotes polymorphisms which are 3′ of the exon that are within the intronic region. The number corresponding to the “+” is given from the 5′ to 3′ direction. Columns 2 and 3, combined, show the SNP names as described herein, e.g., T+1, T+2, etc. Column 4 indicates whether the SNP was detected in an exon or intron sequence. Column 5 lists the SNP locations in the Gene 216 genomic sequence of SEQ ID NO:6 (FIG. 7). Column 6 lists the SNP reference sequences which illustrate the SNP nucleotide changes with underlining. Column 7 lists the SEQ ID NOs of the SNP reference sequences. Column 8 lists the base changes of the SNP sequences. Column 9 lists the amino acid changes resulting from the SNP sequences.

TABLE 10

| | | PMF | | | | SEQ ID | | AA |
|---|---|---|---|---|---|---|---|---|
| SNP | Exon | size | Location | Location | Sequence (20nt+SNP+20nt) | NO | PMP | Change |
| 1 | A | −1 | intron | 4653 | GCCCTCTGAGACCGACGGGGAGGGACGGCTCGGGCCGGGTCA | 241 | A>T | |
| 2 | A | −2 | intron | 4610 | CAAGAACCTTCCCAGCGGTTCTCTCCTCCTCTCAGGAGTAG | 242 | C>A | |
| 3 | C | −1 | intron | 9827 | CACCATCTCAGCTCCACACTCTTTCTTGCCCAGGTCTCGAA | 243 | C>T | |
| 4 | C | −2 | intron | 9826 | CCACCATCTCAGCTCCACACTCTTTCTTGCCCAGGTCTCGA | 244 | T>A | |
| 5 | D | −1 | intron | 11687 | ACAACTAAGCCATCACCAAGGCTCCTTCCTCTAGCCCCAAG | 245 | G>C | |
| 6 | D | −2 | intron | 11661 | TGGTGCTTCCCATATTCACATCTCCCACAACTAAGCCATCA | 246 | T>C | |
| 7 | D | 1 | | 11912 | CAGGATACATAGAAACCCACTACGGCCCAGATGGGCAGCCA | 247 | T>C | Tyr>His |
| 8 | F | +1 | intron | 12545 | CCCTCCAAATCAGAAGAGACAGGAATTCACAGGCCTCGAGT | 248 | A>G | |
| 9 | F | 1 | | 12411 | AGCTGCTCACCTGGAAAGGAACCTGTGGCCACAGGGATCCT | 249 | A>G | Thr>Ala |
| 10 | G | −1 | intron | 12637 | ACTTCCTTCTGGGAGCTGGGGTTGGGGGTCAGGGCTCAAGC | 250 | G>A | |
| 11 | I | 1 | | 13197 | TTCCTGCAGTGGCGCCGGGGGCTGTGGGCGCAGCGGCCCCA | 251 | G>A | none |
| 12 | L | +1 | intron | 14481 | GGTTCAGGGTGAGGGTTTCGGGGAGCTTGGGAGCCGGCCTG | 252 | G>T | |
| 13 | L | −1 | intron | 14043 | CAGAGAAGCGCGGGGGTTGGGGGACTGTCCCTCCATGCCCA | 253 | G>A | |
| 14 | L | −2 | intron | 13988 | CCCCTCTCTGGGCTCTGCGCGTCTGGCGGCTGTAGCCAAGC | 254 | G>A | |
| 15 | L | 1 | | 14135 | CAGCCGCCGCCAGCTGCGCGCCTTCTTCCGCAAGGGGGGCG | 255 | C>T | Ala>Val |
| 16 | Q | +1 | intron | 16158 | AGTGGCCTCCCAGTCAAGCGAGGGGGTGGATCCCTGCCCCA | 256 | A>T | |
| 17 | Q | 1 | | 15865 | TGCTGGCCATGCTCCTCAGCGTCCTGCTGCCTCTGCTCCCA | 257 | G>A | Val>Ile |
| 18 | Q | 2 | | 15888 | CTGCTGCCTCTGCTCCCAGGGGCCGGCCTGGCCTGGTGTTG | 258 | G>C | |
| 19 | QR | +1 | intron | 16133 | GAAGTAGCTTTGAACAGGAGGTTCCAGTGGCCTCCCAGTCA | 259 | G>T | |
| 20 | QR | +3 | intron | 16361 | GCCTCTGTCTCACCAGTTTTCGGCCCTTTGCCACTTCCTCT | 260 | C>T | |
| 21 | QR | +4 | intron | 16404 | ACAAATCACCTCTGTCACCCCCTTGAAGTTCCCAAATGCTG | 261 | C>A | |
| 22 | QR | +5 | intron | 16465 | TCCATACCACTGGTCAGCTGCGGTGCTGGCTGCCCCTGTGC | 262 | C>T | |
| 23 | QR | +6 | intron | 16486 | GGTGCTGGCTGCCCCTGTGCCAGGGCCCTGCCTTAACCCAG | 263 | C>T | |
| 24 | QR | +7 | intron | 16936 | GGAAATGACAAGGCCTTGGGGGATGGGATGGGGACAGTCAA | 264 | G>A | |
| 25 | R | +1 | intron | 17510 | AGGGCTCATGCCTCCTGCCTCCTTCCAGATGGGCAGCACCC | 265 | C>T | |
| 26 | R | +2 | intron | 17571 | GCCCCTCCCCAGCCCCAGGGTCTCCTGCTGACCATATTCAC | 266 | T>G | |
| 27 | R | 1 | | 17403 | CCTGGGCGGCGTTCACCCCATGGAGTTGGGCCCCACAGCCA | 267 | T>C | Met>Thr |
| 28 | R | 2 | | 17432 | GCCCCACAGCCACTGGACAGCCCTGGCCCCTGGGTGAGTGA | 268 | C>T | Pro>Ser |
| 29 | RS | −1 | intron | 17451 | GCCCTGGCCCCTGGGTGAGTGAGGCACCAGGGGGAGGTGGA | 269 | G>T | |
| 30 | T | +1 | intron | 17958 | TGCAGCCTGGGGCCCCAGTCCTTAGGGGACAACATATCCTC | 270 | C>A | |
| 31 | T | +2 | intron | 17924 | CACTGAGTGAGGATGGGCTCTCTGCCACACAGCTTGCAGCC | 271 | T>C | |
| 32 | T | +3 | intron | 17916 | CTGGTCCTCACTGAGTGAGGATGGGCTCTCTGCCACACAGC | 272 | A>G | |
| 33 | T | +4 | intron | 17834 | ATGACCTCTTGGTTATCATGGAGACCAGGATGCTGGAAGCC | 273 | G>C | |
| 34 | T | 1 | 3′UTR | 18833 | AGCAAGACACCGCATCTACAGAAAAATTTTAAAATTAGCTG | 274 | G>A | |
| 35 | T | 2 | | 18787 | GGAGGATCACCAGAGGCCAGCAGGTCCACACCAGCCTGGGC | 275 | C>G | |
| 36 | T | 3 | 3′UTR | 18760 | ATCCCAGCACTTTGGGAAGCCGGGGTAGGAGGATCACCAGA | 276 | C>T | |
| 37 | T | 4 | 3′UTR | 18497 | AGCCTGGCTGGCCTCTGCAAACAAACATAATTTTGGGGACC | 277 | A>G | |
| 38 | T | 5 | | 18476 | ACTGAGTCCACACTCCCCTGCAGCCTGGCTGGCCTCTGCAA | 278 | C>G | |
| 39 | T | 6 | | 18206 | TCCAGGAACCCAGAGCCACATTAGAAGTTCCTGAGGGGCTGG | 279 | T>C | |
| 40 | T | 7 | | 18174 | TTCTTCCCCGAGTGGAGCTTCGACCCACCCACTCCAGGAAC | 280 | C>T | |
| 41 | T | 8 | | 17997 | TCCTCATTCTCAGCAGATCAAGTCCAGATGCCAAGATCCTG | 281 | A>T | Gln>His |
| 42 | T | −2 | intron | 19094 | CTGAGGACCACACGGGGTGGTGGTTGGCGGGGTGGTGGTTG | 282 | T>C | |
| 43 | T | −4 | intron | 19160 | GGCTGGCAGGCCGAGCCTAGATGGCAGCCAGAGCCCCAGGC | 283 | A>G | |
| 44 | T | −5 | intron | 19244 | CTTTGCTCTGTCACTCCTGCCTCCCTTGGGCGTTCACATTC | 284 | C>T | |
| 45 | U | −1 | intron | 15423 | GTGAGCTCTGCCCACCCGACCCCTCCTTGCCGTTTGAATCC | 285 | C>T | |
| 46 | V | +1 | intron | 13859 | TGGCGAGGTTACTCCTACACCGGGAGGAGCACCGTCGGGTC | 286 | C>T | |
| 47 | V | +2 | intron | 13921 | GGCTGCTCACTATTGGGGCCGCATCGTCCCCTGTCCCGCTT | 287 | G>T | |
| 48 | V | +3 | intron | 13938 | GCCGCATCGTCCCCTGTCCCGCTGTTGTGTGACTTTGCGC | 288 | G>A | |

Using an in-house program called snp_view; the genomic structure of the gene is diagrammatically shown in FIG. 11. The exons are shown to scale and the SNPs are identified by their location along the genomic BAC DNA. The polymorphic sites identified in the Gene 216 genomic sequence are also shown by the underlined nucleotides in FIG. 29. The polymorphic sites discovered within the cDNA and the corresponding amino acid position in Gene 216 are underlined in FIG. 24. It will be understood by those of skill in the art that the SNPs identified in the Gene 216 genomic sequence can be correlated to the SNP positions identified in the Gene 216 cDNA sequence by aligning the genomic and cDNA sequences.

### EXAMPLE 11

### Polymorphism Genotyping

Once putative variants were confirmed by sequencing, rapid allele specific assays were designed to type more than 400 individuals (>200 cases and >200 controls) for use in the association studies. All coding SNPs (cSNPs) that resulted in an amino acid change were typed. Neutral polymorphisms were typed if: 1) the polymorphism was present in an exon lacking a cSNP that resulted in an amino acid change; 2) the polymorphism was present in an exon containing a cSNP resulting in an amino acid change but the two polymorphisms were observed to have different frequencies; and 3) the polymorphism was in an intronic region adjacent to an exon without a cSNP. If results from the association studies appeared positive, additional neutral polymorphisms were typed. More than 30 allele specific assays from Gene 216 were typed for the case control population (Table 11).

Two types of allele specific assays (ASAs) were used. If the SNP resulted in a mutation that created or abolished a restriction site, restriction fragment length polymorphisms (RFLPs) were obtained from PCR products that spanned the variants, and the RFLPs were analyzed. If the polymorphisms did not result in RFLPs, allele specific oligonucleotide assays were used. For these assays, PCR products that spanned the polymorphism were electrophoresed on agarose gels and transferred to nylon membranes by Southern blotting. Oligomers 16-20 bp in length were designed such that the middle base was specific for each variant. The oligomers were labeled and successively hybridized to the membrane in order to determine genotypes. The specific method used to type each SNP is indicated in Table 11.

Table 11 below contains the information relating to the specific assay used. Column 1 lists the SNP designation number. Column 2 lists the specific assay used, either RFLP or ASO. Column 3 lists the enzyme used in the RFLP assay (described below). Columns 4 and 6 list the sequence of the primers used in the ASO assay (described below). Columns 5 and 7 list the corresponding SEQ ID NOS for the primers.

1. RFLP Assay:

The amplicon containing the polymorphism was PCR amplified using primers that were used to generate a fragment for sequencing (sequencing primers) or SSCP(SSCP primers). The appropriate population of individuals was PCR amplified in 96 well microtitre plates.

Enzymes were purchased from NEB. The restriction cocktail containing the appropriate enzyme for the particular polymorphism is added to the PCR product. The reaction was incubated at the appropriate temperature according to the manufacturer's recommendations (NEB) for 2-3 hr, followed by a 4° C. incubation. After digestion, the reactions were size fractionated using the appropriate agarose gel depending on the assay specifications (2.5%, 3%, or Metaphor, FMC Bioproducts). Gels were electrophoresed in 1× TBE Buffer at 170 Volts for approximately 2 hr. The gel was illuminated using ultraviolet light and the image was saved as a Kodak 1 D file. Using the Kodak 1 D image analysis software, the images were scored and the data was exported to Microsoft EXCEL (http://www.microsoft.com).

2. ASO Assay:

The amplicon containing the polymorphism was PCR amplified using primers that were used to generate a fragment for sequencing (sequencing primers) or SSCP(SSCP primers). The appropriate population of individuals was PCR amplified in 96 well microtitre plates and re-arrayed into 384 well microtitre plates using a Tecan Genesis RSP200. The amplified products were loaded onto 2% agarose gels and size fractionated at 150V for 5 min. The DNA was transferred from the gel to Hybond N+ nylon membrane (Amersham-Pharmacia) using a Vacuum blotter (Bio-Rad). The filter containing the blotted PCR products was transferred to a dish containing 300 ml pre-hybridization solution (5× SSPE (pH 7.4), 2% SDS, 5× Denhardt's). The filter was incubated in pre-hybridization solution at 40° C. for over 1 hr. After pre-hybridization, 10 ml of the pre-hybridization solution and the filter were transferred to a washed glass bottle. The allele specific oligonucleotides (ASO) were designed with the polymorphism in the middle. The size of the oligonucleotide was dependent upon the GC content of the sequence around the polymorphism. Those ASOs that had a G or C polymorphism were designed so that the Tₘ was between 54-56° C. and those that had an A or T variance were designed so that the Tm was between 60-64° C. All oligonucleotides were phosphate free at the 5′ end and purchased from GibcoBRL. For each polymorphism, 2 ASOs were designed: one for each variant.

The two ASOs that represented the polymorphism were resuspended at a concentration of 1 μg/μl and separately end-labeled with γ-ATP³² (6000 Ci/mmol) (NEN) using T4 polynucleotide kinase according to manufacturer recommendations (NEB). The end-labeled products were removed from the unincorporated γ-ATP³² by passing the reactions through Sephadex G-25 columns according to manufacturers recommendation (Amersham-Pharmacia). The entire end-labeled product of one ASO was added to the bottle containing the appropriate filter and 10 ml hybridization solution. The hybridization reaction was placed in a rotisserie oven (Hybaid) and left at 40° C. for a minimum of 4 hr. The other ASO was stored at −20° C.

After the prerequisite hybridization time had elapsed, the filter was removed from the bottle and transferred to 1 L of wash solution (0.1× SSPE (pH 7.4), 0.1% SDS) pre-warmed to 45° C. After 15 min, the filter was transferred to another L of wash solution (0.1× SSPE (pH 7.4), 0.1% SDS) pre-warmed to 50° C. After 15 min, the filter was wrapped in Saran, placed in an autoradiograph cassette and an X-ray film (Kodak) placed on top of the filter. Typically, an image would be observed on the film within 1 hr. After an image had been captured on film for the 50° C. wash, the process was repeated for wash steps at 55° C., 60° C. and 65° C. The image that captured the best result was used.

The ASO was removed from the filter by adding 1 L of boiling strip solution (0.1× SSPE (pH 7.4), 0.1% SDS). This was repeated two more times. After removing the ASO the filter was pre-hybridized in 300 ml pre-hybridization solution (5× SSPE (pH 7.4), 2% SDS, 5× Denhardt's) at 40° C. for over 1 hr. The second end-labeled ASO corresponding to the other variant was removed from storage at −20° C. and thawed at room temperature. The filter was placed into a glass bottle along with 10 ml hybridization solution and the entire end-labeled product of the second ASO. The hybridization reaction was placed in a rotisserie oven (Hybaid, http://www.hybaid.co.uk) and left at 40° C. for a minimum of 4 hr. After the hybridization, the filter was washed at various temperatures and images captured on film as described above.

The two films that best captured the allele-specific assay with the two ASOs were converted into digital images by scanning them into Adobe PhotoShop. These images were overlaid against each other in Graphic Converter and then scored.

TABLE 11

| | ASA | RFLP | | SEQ ID | | |
|---|---|---|---|---|---|---|
| SNP | Type | Enzyme | ASO Primer 1 | NO: | ASO Primer 2 | SEQ ID NO: |
| 1 | ASO | | gccgtcccaccccgtcg | 289 | gccgtccctccccgtcg | 299 |
| 2 | ASO | | cctcctctcttggcgac | 290 | tcctcctctattggcgaccc | 300 |
| 3 | ASO | | tccacactctttcttgcc | 291 | ctccacactttttcttgccca | 301 |
| 4 | ASO | | gctccacactctttcttgcc | 292 | gctccacactctttcttgc | 302 |
| 5 | ASO | | tcaccaaggctccttcct | 293 | tcaccaagcctccttcct | 303 |
| 6 | Alt. Meth | | | | | |
| 7 | RFLP | XcmI | | | | |
| 8 | ASO | | cagaagagacaggaattcaca | 294 | agaagagacgggaattcac | 304 |
| 9 | ASO | | tggaaaggaacctgtggcc | 295 | tggaaaggagcctgtgg | 305 |
| 10 | ASO | | | | | |
| 11 | ASO | | | | | |
| 12 | ASO | | gggtttcggggagcttg | 296 | agggtttcgtggagcttgg | 306 |
| 13 | ASO | | gggttgggggactgtc | 297 | ggggttggaggactgtcc | 307 |
| 14 | ASO | | ctctgcgcgtctggcg | 298 | gctctgcgcatctggcgg | 308 |
| 15 | RFLP | BssHII | | | | |
| 16 | ASO | | agtcaagcgagggggtgg | 309 | agtcaagcgtgggggtgg | 322 |
| 17 | ASO | | cctcagcgtcctgctg | 310 | ctcctcagcatcctgctgc | 323 |
| 18 | RFLP | KasI | | | | |
| 19 | ASO | | aacaggaggttccagtgg | 311 | gaacaggagtttccagtggc | 324 |
| 20 | ASO | | accagttttcggcccttt | 312 | caccagtttttggccctttg | 325 |
| 21 | ASO | | ctgtcacccccttgaagt | 313 | ctgtcacccacttgaagttc | 326 |
| 22 | ASO | | tcagctgcggtgctgg | 314 | ggtcagctgtggtgctgg | 327 |
| 23 | RFLP | BstNI | | | | |
| 24 | ASO | | gccttgggggatgga | 315 | aggccttgggagatgggat | 328 |
| 25 | ASO | | tcctgcctccttccag | 316 | tcctgccttcttccag | 329 |
| 26 | RFLP | BgII | | | | |
| 27 | RFLP | NcoI | | | | |
| 28 | ASO | | actggacagccctggc | 317 | actggacagtcctggc | 330 |
| 29 | ASO | | | | | |
| 30 | RFLP | Bsu36I | | | | |
| 31 | ASO | | ctgtgtggcagagagccca | 318 | tgtggcagggagccca | 331 |
| 32 | ASO | | | | | |
| 33 | RFLP | BsaI | | | | |
| 34 | Alt. Meth | | | | | |
| 35 | RFLP | Cac8I | | | | |
| 36 | RFLP | MspI | | | | |
| 37 | ASO | | aattatgtttgtttgcagaggc | 319 | attatgtttgcttgcagagg | 332 |
| 38 | RFLP | Fnu4HI | | | | |
| 39 | ASO | | gaacttctagtgtggctct | 320 | ggaacttctaatgtggctctg | 333 |
| 40 | RFLP | TaqI | | | | |
| 41 | RFLP | NlaIII | | | | |
| 42 | ASO | | | | | |
| 43 | RFLP | StyI | | | | |
| 44 | ASO | | ccaagggaggcaggagt | 321 | cccaagggaagcaggagtga | 334 |
| 45 | RFLP | HinfI | | | | |
| 46 | RFLP | BsrI | | | | |

| | | |
|---|---|---|
| 47 | RFLP | Eco109I |
| 48 | ASO | |

### EXAMPLE 12

### Association Study Analysis

1. Case-Control Study:

In order to determine whether polymorphisms in candidate genes were associated with the asthma phenotype, association studies were performed using a case-control study design. In a well-matched design, the case-control approach is more powerful than the family based transmission disequilibrium test (TDT) (N.E. Morton and A. Collins, 1998, Proc. Natl. Acad. Sci. USA 95:11389-93). Case-control studies are, however, sensitive to population heterogeneity.

To avoid issues of population admixture, which can bias case-control studies, the unaffected controls were collected in both the US and the UK. A total of three hundred controls were collected, 200 in the UK and 100 in the US. Inclusion into the study required that the control individual was negative for asthma, as determined by self-report of never having asthma, had no first degree relatives with asthma, and was negative for eczema and symptoms indicative of atopy within the past 12 months. Data from an abbreviated questionnaire similar to that administered to the affected sib pair families were collected. Results from skin prick tests to 4 common allergens were also collected. The results of the skin prick test were used to select a subset of controls that were most likely to be asthma and atopy negative.

A subset of unrelated cases was selected from the affected sib pair families based on the evidence for linkage at the chromosomal location near a given gene. One affected sib demonstrating identity-by-descent (IBD) at the appropriate marker loci was selected from each family. Since the appropriate cases may vary for each gene in the chromosome 20 region, a larger collection of individuals who were IBD across a larger interval were genotyped, and a subset was used in the analyses. On average, 130 IBD affected individuals and 200 controls were compared for allele and genotype frequencies. This number provided an 80% power to detect a difference of 5% or greater between the two groups for a rare allele (≦5%) at a 0.05 level of significance. For a common allele (50%), the number provided an 80% power to detect a difference of 10% or more between the two groups.

For each polymorphism, the frequency of the alleles in the control and case populations was compared using a Fisher exact test. A mutation that increased susceptibility to the disease would be more prevalent in the cases than in the controls, while a protective mutation would be more prevalent in the control group. Similarly, the genotype frequencies of the SNPs were compared between cases and controls. P-values for both the allele and genotype were plotted against a coordinate system based on genomic sequence to visualize regions where allelic association was present. A small p-value (or a large value of -log (p) as plotted in the figures described below) was indicative of an association between the SNPs and the disease phenotype. The analysis was repeated for the US and UK population separately to adjust for the possibility of genetic heterogeneity.

2. Association Test with Individual SNPs:

Chromosomal regions harboring asthma susceptibility genes were identified by association studies using the SNP typing data. Two separate phenotypes were used in these analyses: asthma and bronchial hyper-responsiveness.

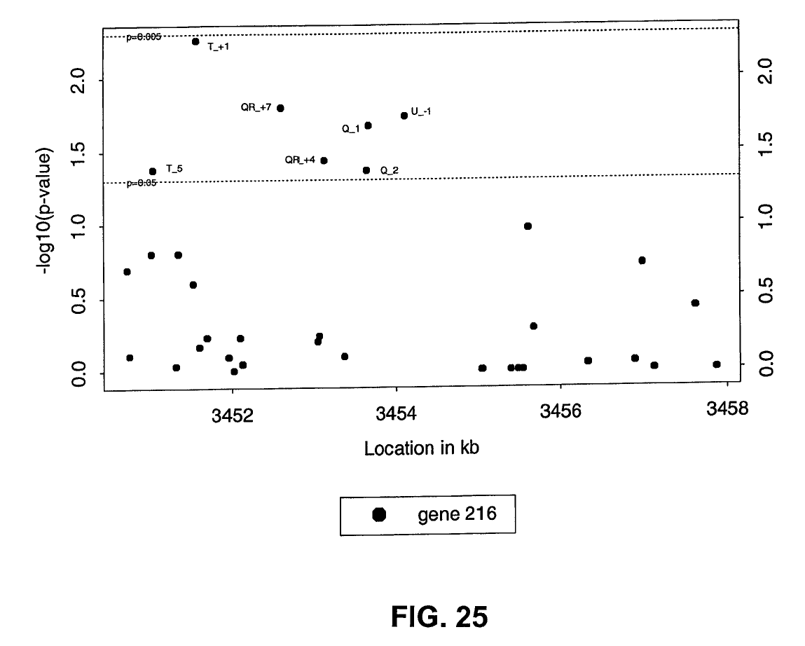

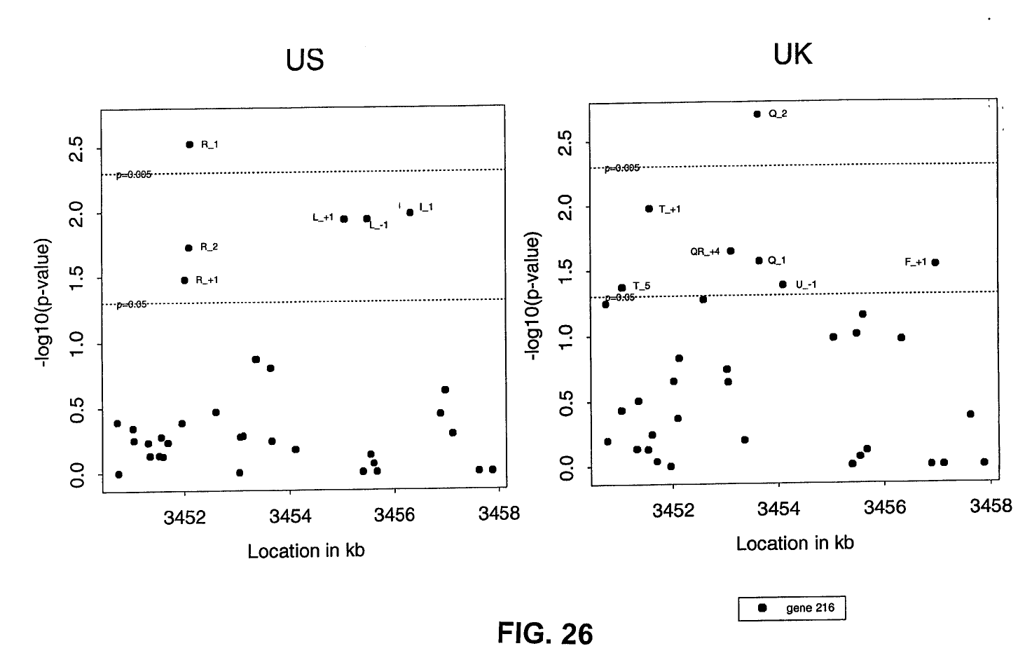

a. Asthma Phenotype: The significance levels (p-values) for allelic association of all typed SNPs in Gene 216 to the asthma phenotype are plotted in FIG. 25 (combined population) and FIG. 26 (US and UK populations separately). The most significant result in the combined population was observed for Gene 216 exon T+1, where 92.4% of the cases harbored the intronic mutation, while the SNP was present in only 85.2% of the controls (p=0.0055). Six additional SNPs in Gene 216 (T5, QR+7, QR+4, Q2, Q1, and U−1) were significant at the 0.05 level. Frequencies and p-values for SNPs associated with the asthma phenotype in Gene 216 are presented in Tables 12, 13, and 14 for the combined population and for the UK and US populations, separately.

TABLE 12
Asthma Yes/NO
Combined US and UK

| | Frequencies | | | | ALLELE | GENOTYPE |
|---|---|---|---|---|---|---|
| GENE_EXON | CNTL | N | CASE | N | P-VALUE | P-VALUE |
| gene216_T_2 | 66.5% | 215 | 71.5% | 128 | 0.2029 | 0.1482 |
| gene216_T_3 | 8.7% | 213 | 9.5% | 131 | 0.7841 | 0.6895 |
| gene216_T_4 | 96.3% | 215 | 98.5% | 129 | 0.1576 | 0.1513 |
| gene216_T_5 | 76.7% | 217 | 83.3% | 129 | 0.0420 | 0.0468 |
| gene216_T_6 | 77.8% | 214 | 78.4% | 125 | 0.9235 | 0.9791 |
| gene216_T_7 | 96.3% | 215 | 98.5% | 129 | 0.1576 | 0.1513 |
| gene216_T_8 | 96.5% | 211 | 98.1% | 129 | 0.2528 | 0.2456 |
| gene216_T_+1 | 85.2% | 216 | 92.4% | 131 | 0.0055 | 0.0178 |
| gene216_T_+2 | 37.3% | 209 | 39.0% | 127 | 0.6825 | 0.7722 |
| gene216_T_+4 | 24.4% | 215 | 26.3% | 131 | 0.5886 | 0.7410 |
| gene216_R_+2 | 88.3% | 217 | 88.9% | 131 | 0.8076 | 0.9005 |
| gene216_R_+1 | 88.7% | 191 | 88.8% | 120 | 1.0000 | 0.8394 |
| gene216_R_2 | 9.4% | 208 | 10.8% | 125 | 0.5928 | 0.7656 |
| gene216_R_1 | 11.3% | 217 | 11.8% | 131 | 0.9025 | 0.7483 |
| gene216_QR_+7 | 78.1% | 215 | 85.7% | 129 | 0.0160 | 0.0265 |
| gene216_QR_+6 | 0.5% | 216 | 0.8% | 129 | 0.6323 | 0.6317 |
| gene216_QR_+5 | 46.4% | 210 | 48.8% | 129 | 0.5794 | 0.4165 |
| gene216_QR_+4 | 51.5% | 205 | 59.9% | 126 | 0.0367 | 0.1272 |
| gene216_Q_+1 | 51.2% | 206 | 52.5% | 120 | 0.8075 | 0.6608 |
| gene216_Q_2 | 73.7% | 217 | 80.5% | 131 | 0.0432 | 0.0831 |
| gene216_Q_1 | 89.5% | 209 | 94.8% | 125 | 0.0213 | 0.0584 |
| gene216_U_−1 | 85.0% | 217 | 91.2% | 131 | 0.0184 | 0.0659 |
| gene216_L_+1 | 88.7% | 213 | 88.9% | 131 | 1.0000 | 0.9672 |
| gene216_L_1 | 99.3% | 217 | 99.6% | 131 | 1.0000 | 1.0000 |
| gene216_L_−1 | 88.9% | 212 | 89.2% | 130 | 1.0000 | 1.0000 |
| gene216_L_−2 | 92.9% | 212 | 93.1% | 131 | 1.0000 | 0.9379 |
| gene216_V_+2 | 71.3% | 216 | 77.1% | 129 | 0.1085 | 0.2262 |
| gene216_V_+1 | 96.1% | 217 | 97.2% | 125 | 0.5223 | 0.5145 |
| gene216_I_1 | 84.9% | 212 | 85.3% | 129 | 0.9124 | 1.0000 |
| gene216_G_−1 | 90.7% | 210 | 91.3% | 127 | 0.8900 | 0.7683 |
| gene216_F_+1 | 65.2% | 197 | 70.4% | 120 | 0.1913 | 0.4109 |
| gene216_F_1 | 96.8% | 217 | 96.9% | 129 | 1.0000 | 1.0000 |
| gene216_D_1 | 0.0% | 215 | 0.4% | 131 | 0.3786 | 0.3786 |
| gene216_D_−2 | 0.7% | 214 | 0.8% | 127 | 1.0000 | 1.0000 |

TABLE 13
Asthma Yes/No
UK population

| | Frequencies | | | | ALLELE | GENOTYPE |
|---|---|---|---|---|---|---|
| GENE_EXON | CNTL | N | CASE | N | P-VALUE | P-VALUE |
| gene216_T_2 | 65.8% | 139 | 74.3% | 101 | 0.0566 | 0.1266 |
| gene216_T_3 | 8.3% | 139 | 9.6% | 104 | 0.6308 | 0.7329 |
| gene216_T_4 | 97.1% | 140 | 98.5% | 103 | 0.3689 | 0.3633 |
| gene216_T_5 | 75.4% | 140 | 83.3% | 102 | 0.0426 | 0.0365 |
| gene216_T_6 | 78.5% | 137 | 80.1% | 98 | 0.7301 | 0.8875 |
| gene216_T_7 | 97.5% | 138 | 99.0% | 102 | 0.3129 | 0.3082 |
| gene216_T_8 | 97.8% | 137 | 98.5% | 102 | 0.7388 | 0.7363 |
| gene216_T_+1 | 86.4% | 140 | 93.8% | 104 | 0.0105 | 0.0243 |
| gene216_T_+2 | 37.9% | 136 | 40.5% | 100 | 0.5682 | 0.8375 |
| gene216_T_+4 | 25.2% | 139 | 26.0% | 104 | 0.9163 | 0.6037 |
| gene216_R_+2 | 87.5% | 140 | 87.5% | 104 | 1.0000 | 1.0000 |
| gene216_R_+1 | 86.9% | 122 | 91.1% | 95 | 0.2211 | 0.4281 |
| gene216_R_2 | 10.5% | 134 | 8.2% | 98 | 0.4279 | 0.7007 |
| gene216_R_1 | 13.2% | 140 | 8.7% | 104 | 0.1473 | 0.3472 |
| gene216_QR_+7 | 79.5% | 139 | 86.4% | 103 | 0.0535 | 0.1362 |
| gene216_QR_+6 | 0.0% | 139 | 1.0% | 103 | 0.1806 | 0.1801 |
| gene216_QR_+5 | 44.4% | 133 | 50.0% | 102 | 0.2273 | 0.2470 |
| gene216_QR_+4 | 48.1% | 128 | 59.1% | 99 | 0.0229 | 0.0730 |
| gene216_Q_+1 | 53.1% | 129 | 50.5% | 97 | 0.6346 | 0.5458 |
| gene216_Q_2 | 72.9% | 140 | 84.6% | 104 | 0.0020 | 0.0050 |
| gene216_Q_1 | 89.4% | 132 | 95.1% | 101 | 0.0274 | 0.0732 |
| gene216_U_−1 | 86.1% | 140 | 92.3% | 104 | 0.0419 | 0.0763 |
| gene216_L_+1 | 87.0% | 138 | 91.8% | 104 | 0.1059 | 0.2969 |
| gene216_L_1 | 99.3% | 140 | 99.5% | 104 | 1.0000 | 1.0000 |
| gene216_L_−1 | 87.2% | 137 | 92.2% | 103 | 0.0992 | 0.1655 |
| gene216_L_−2 | 92.7% | 137 | 92.3% | 104 | 0.8633 | 1.0000 |
| gene216_V_+2 | 71.6% | 139 | 79.1% | 103 | 0.0717 | 0.1519 |
| gene216_V_+1 | 97.1% | 140 | 98.0% | 99 | 0.7685 | 0.7655 |
| gene216_I_1 | 83.7% | 138 | 89.2% | 102 | 0.1094 | 0.1323 |
| gene216_G_−1 | 90.2% | 137 | 90.1% | 101 | 1.0000 | 0.4913 |
| gene216_F_+1 | 64.1% | 128 | 74.2% | 93 | 0.0295 | 0.0711 |
| gene216_F_1 | 97.9% | 140 | 98.0% | 102 | 1.0000 | 1.0000 |
| gene216_D_1 | 0.0% | 139 | 0.5% | 104 | 0.4280 | 0.4280 |
| gene216_D_−2 | 0.7% | 139 | 1.0% | 101 | 1.0000 | 1.0000 |

TABLE 14
Asthma Yes/No
US population

| | Fre- | | | | | GENO- |
|---|---|---|---|---|---|---|
| | quencies | | | | ALLELE | TYPE |
| GENE_EXON | CNTL | N | CASE | N | P-VALUE | P-VALUE |
| gene216_T_2 | 67.8% | 76 | 61.1% | 27 | 0.4053 | 0.1776 |
| gene216_T_3 | 9.5% | 74 | 9.3% | 27 | 1.0000 | 1.0000 |
| gene216_T_4 | 94.7% | 75 | 98.1% | 26 | 0.4519 | 0.4404 |
| gene216_T_5 | 79.2% | 77 | 83.3% | 27 | 0.5583 | 0.7765 |
| gene216_T_6 | 76.6% | 77 | 72.2% | 27 | 0.5819 | 0.6932 |
| gene216_T_7 | 94.2% | 77 | 96.3% | 27 | 0.7320 | 0.7241 |
| gene216_T_8 | 93.9% | 74 | 96.3% | 27 | 0.7308 | 0.7226 |
| gene216_T_+1 | 82.9% | 76 | 87.0% | 27 | 0.5262 | 0.8281 |
| gene216_T_+2 | 36.3% | 73 | 33.3% | 27 | 0.7416 | 0.5739 |
| gene216_T_+4 | 23.0% | 76 | 27.8% | 27 | 0.5795 | 0.6743 |
| gene216_R_+2 | 89.6% | 77 | 94.4% | 27 | 0.4127 | 0.3874 |
| gene216_R_+1 | 92.0% | 69 | 80.0% | 25 | 0.0334 | 0.0361 |
| gene216_R_2 | 7.4% | 74 | 20.4% | 27 | 0.0188 | 0.0208 |
| gene216_R_1 | 7.8% | 77 | 24.1% | 27 | 0.0030 | 0.0055 |
| gene216_QR_+7 | 75.7% | 76 | 82.7% | 26 | 0.3410 | 0.0921 |
| gene216_QR_+6 | 1.3% | 77 | 0.0% | 26 | 1.0000 | 1.0000 |
| gene216_QR_+5 | 50.0% | 77 | 44.4% | 27 | 0.5287 | 0.6337 |
| gene216_QR_+4 | 57.1% | 77 | 63.0% | 27 | 0.5218 | 0.4709 |
| gene216_Q_+1 | 48.1% | 77 | 60.9% | 23 | 0.1345 | 0.3169 |
| gene216_Q_2 | 75.3% | 77 | 64.8% | 27 | 0.1571 | 0.1404 |
| gene216_Q_1 | 89.6% | 77 | 93.8% | 24 | 0.5726 | 1.0000 |
| gene216_U_−1 | 83.1% | 77 | 87.0% | 27 | 0.6654 | 0.8280 |
| gene216_L_+1 | 92.0% | 75 | 77.8% | 27 | 0.0116 | 0.0123 |
| gene216_L_1 | 99.4% | 77 | 100.0% | 27 | 1.0000 | 1.0000 |
| gene216_L_−1 | 92.0% | 75 | 77.8% | 27 | 0.0116 | 0.0123 |
| gene216_L_−2 | 93.3% | 75 | 96.3% | 27 | 0.7362 | 0.5089 |
| gene216_V_+2 | 70.8% | 77 | 69.2% | 26 | 0.8614 | 0.8889 |
| gene216_V_+1 | 94.2% | 77 | 94.2% | 26 | 1.0000 | 1.0000 |
| gene216_I_1 | 87.2% | 74 | 70.4% | 27 | 0.0105 | 0.0074 |
| gene216_G_−1 | 91.8% | 73 | 96.2% | 26 | 0.3635 | 0.3440 |
| gene216_F_+1 | 67.4% | 69 | 57.4% | 27 | 0.2401 | 0.3270 |
| gene216_F_1 | 94.8% | 77 | 92.6% | 27 | 0.5136 | 0.5043 |
| gene216_D_1 | 0.0% | 76 | 0.0% | 27 | 1.0000 | 1.0000 |
| gene216_D_−2 | 0.7% | 75 | 0.0% | 26 | 1.0000 | 1.0000 |

b. Bronchial Hyper-Responsiveness:

The analyses were repeated using asthmatic children with borderline to severe BHR(PC₂₀≦16 mg/ml) or PC₂₀(16), as described in the linkage section. First, sibling pairs were identified where both sibs were affected and satisfied this new criteria. Of these pairs, one sib was included in the case/control analyses if they showed evidence of linkage at the gene of interest. This phenotype was more restrictive than the Asthma yes/no criteria; hence the number of cases included in the analyses was reduced approximately in half. If the PC₂₀(16) subgroup represented a more genetically homogeneous sample, one expected to see an increase in the effect size compared to the one observed in the original set of cases. However, the reduction in sample size could result in estimates that were less accurate and that could obscure a trend in allele frequencies in the control group, the original set of cases and the PC₂₀(16) subgroup. In addition, the reduction in sample size could induce a reduction in power (and increase in p values) in spite of the larger effect size.

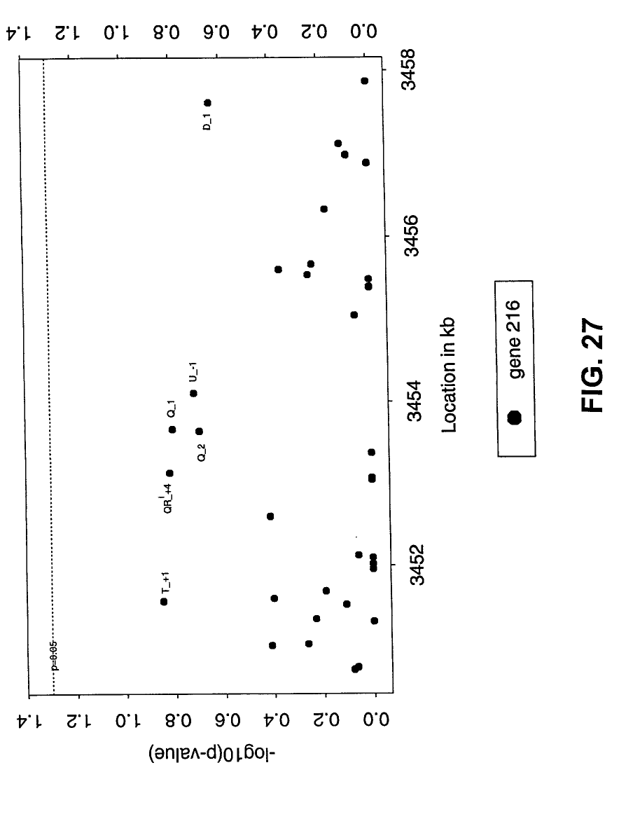

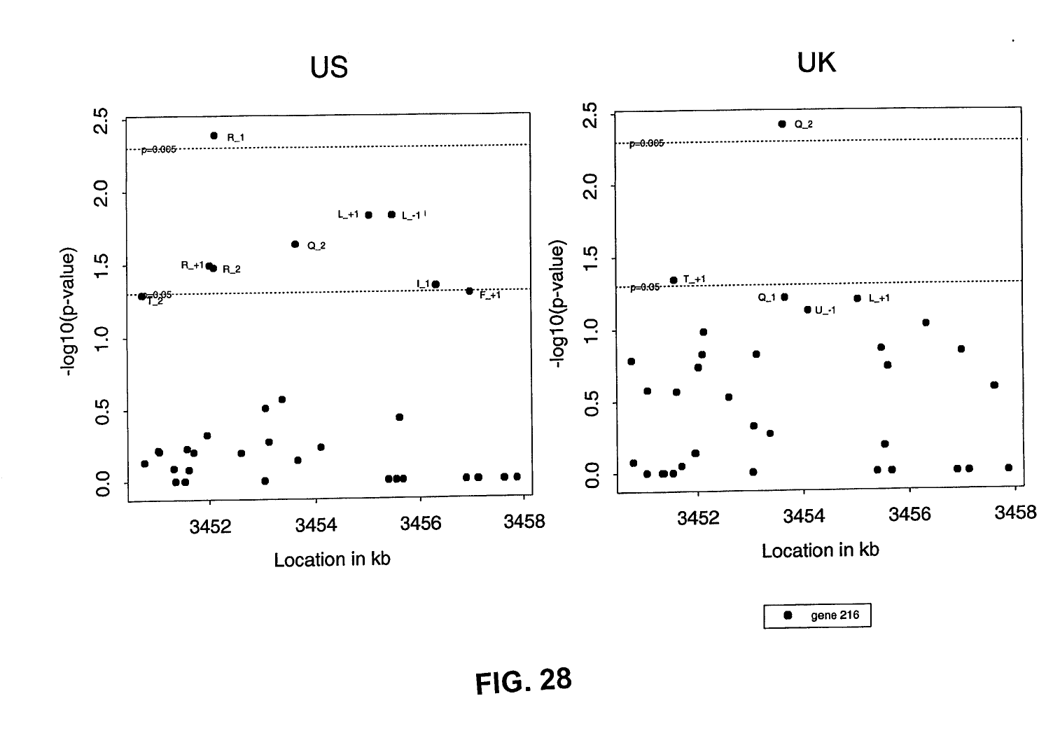

The significance levels (p-values) for allelic association of all typed SNPs in Gene 216 to the BHR phenotype are plotted in FIG. 27 (combined population) and FIG. 28 (US and UK populations separately). Frequencies and p-values for SNPs associated with the BHR phenotype in Gene 216 are presented in Tables 15, 16, and 17 for the combined population and for the UK and US populations, separately. Again, multiple SNPs in Gene 216 were associated with the phenotype in each separate population. In the UK population, the most significant SNP was in Gene 216, exon Q2, where 87% of the cases had the mutation compared to 72.9% for the controls (p=0.0038). For the US population, the most significant association was found with the SNP in Gene 216 exon R1, where 28.6% of the cases carried the mutation compared to 7.8% for the controls (p=0.0041).

In summary, Gene 216 associated with the phenotypes of both asthma and bronchial hyper-responsiveness. Association was found with multiple SNPs in both the UK and US populations. The 3′ region of the gene, which contains the transmembrane domain, the cytoplasmic domain, and the 3′ UTR, appeared to have the strongest association. Taken together, these data strongly suggested that Gene 216 is an asthma susceptibility gene.

TABLE 15
BHR
Combined US and UK

| | Frequencies | | | | ALLELE | GENOTYPE |
|---|---|---|---|---|---|---|
| GENE_EXON | CNTL | N | CASE | N | P-VALUE | P-VALUE |
| gene216_T_2 | 66.5% | 215 | 67.7% | 62 | 0.8294 | 0.1358 |
| gene216_T_3 | 8.7% | 213 | 9.4% | 64 | 0.8592 | 0.6092 |
| gene216_T_4 | 96.3% | 215 | 98.4% | 62 | 0.3878 | 0.3797 |
| gene216_T_5 | 76.7% | 217 | 79.8% | 62 | 0.5428 | 0.5315 |
| gene216_T_6 | 77.8% | 214 | 78.3% | 60 | 1.0000 | 0.8426 |
| gene216_T_7 | 96.3% | 215 | 97.7% | 64 | 0.5856 | 0.5786 |
| gene216_T_8 | 96.5% | 211 | 97.6% | 63 | 0.7758 | 0.7721 |
| gene216_T_+1 | 85.2% | 216 | 90.6% | 64 | 0.1413 | 0.3117 |
| gene216_T_+2 | 37.3% | 209 | 41.8% | 61 | 0.3978 | 0.6939 |
| gene216_T_+4 | 24.4% | 215 | 26.6% | 64 | 0.6421 | 0.2498 |
| gene216_R_+2 | 88.3% | 217 | 88.3% | 64 | 1.0000 | 0.8975 |
| gene216_R_+1 | 88.7% | 191 | 89.2% | 60 | 1.0000 | 0.7540 |
| gene216_R_2 | 90.6% | 208 | 91.1% | 62 | 1.0000 | 1.0000 |
| gene216_R_1 | 11.3% | 217 | 11.7% | 64 | 0.8750 | 0.7576 |
| gene216_QR_+7 | 78.1% | 215 | 82.0% | 64 | 0.3876 | 0.1711 |
| gene216_QR_+6 | 99.5% | 216 | 100.0% | 63 | 1.0000 | 1.0000 |
| gene216_QR_+5 | 46.4% | 210 | 46.8% | 63 | 1.0000 | 0.5530 |
| gene216_QR_+4 | 51.5% | 205 | 58.9% | 62 | 0.1521 | 0.3393 |
| gene216_Q_+1 | 51.2% | 206 | 51.8% | 57 | 1.0000 | 0.7632 |
| gene216_Q_2 | 73.7% | 217 | 79.7% | 64 | 0.2009 | 0.0664 |
| gene216_Q_1 | 89.5% | 209 | 94.2% | 60 | 0.1565 | 0.4299 |
| gene216_U_−1 | 85.0% | 217 | 89.8% | 64 | 0.1915 | 0.5304 |
| gene216_L_+1 | 88.7% | 213 | 89.8% | 64 | 0.8722 | 0.9410 |
| gene216_L_1 | 0.7% | 217 | 0.8% | 64 | 1.0000 | 1.0000 |
| gene216_L_−1 | 88.9% | 212 | 89.1% | 64 | 1.0000 | 1.0000 |
| gene216_L_−2 | 7.1% | 212 | 8.6% | 64 | 0.5661 | 0.5313 |
| gene216_V_+2 | 71.3% | 216 | 75.0% | 64 | 0.4343 | 0.7291 |
| gene216_V_+1 | 96.1% | 217 | 97.6% | 63 | 0.5874 | 0.5802 |
| gene216_I_1 | 84.9% | 212 | 86.7% | 64 | 0.6709 | 0.8958 |
| gene216_G_−1 | 9.3% | 210 | 9.5% | 63 | 1.0000 | 0.9355 |
| gene216_F_+1 | 65.2% | 197 | 66.7% | 57 | 0.8234 | 0.3665 |
| gene216_F_1 | 96.8% | 217 | 97.6% | 62 | 0.7752 | 0.7715 |
| gene216_D_1 | 0.0% | 215 | 0.8% | 64 | 0.2294 | 0.2294 |
| gene216_D_−2 | 0.7% | 214 | 0.8% | 63 | 1.0000 | 1.0000 |

TABLE 16
BHR
UK population

| | Frequencies | | | | ALLELE | GENOTYPE |
|---|---|---|---|---|---|---|
| GENE_EXON | CNTL | N | CASE | N | P-VALUE | P-VALUE |
| gene216_T_2 | 65.8% | 139 | 74.0% | 48 | 0.1635 | 0.1885 |
| gene216_T_3 | 8.3% | 139 | 9.0% | 50 | 0.8352 | 0.6515 |
| gene216_T_4 | 97.1% | 140 | 98.0% | 49 | 1.0000 | 1.0000 |
| gene216_T_5 | 75.4% | 140 | 81.3% | 48 | 0.2641 | 0.3646 |
| gene216_T_6 | 78.5% | 137 | 79.4% | 46 | 1.0000 | 0.9547 |
| gene216_T_7 | 97.5% | 138 | 98.0% | 50 | 1.0000 | 1.0000 |
| gene216_T_8 | 97.8% | 137 | 98.0% | 49 | 1.0000 | 1.0000 |
| gene216_T_+1 | 86.4% | 140 | 94.0% | 50 | 0.0454 | 0.1307 |
| gene216_T_+2 | 37.9% | 136 | 44.7% | 47 | 0.2715 | 0.4549 |
| gene216_T_+4 | 25.2% | 139 | 26.0% | 50 | 0.8938 | 0.1153 |
| gene216_R_+2 | 87.5% | 140 | 86.0% | 50 | 0.7290 | 0.6834 |
| gene216_R_+1 | 86.9% | 122 | 92.6% | 47 | 0.1838 | 0.3875 |
| gene216_R_2 | 89.6% | 134 | 94.8% | 48 | 0.1494 | 0.4752 |
| gene216_R_1 | 13.2% | 140 | 7.0% | 50 | 0.1041 | 0.3226 |
| gene216_QR_+7 | 79.5% | 139 | 85.0% | 50 | 0.2983 | 0.3872 |
| gene216_QR_+6 | 0.0% | 139 | 0.0% | 49 | 1.0000 | 1.0000 |
| gene216_QR_+5 | 44.4% | 133 | 49.0% | 49 | 0.4771 | 0.5020 |
| gene216_QR_+4 | 48.1% | 128 | 57.3% | 48 | 0.1508 | 0.2350 |
| gene216_Q_+1 | 53.1% | 129 | 48.9% | 45 | 0.5407 | 0.6988 |
| gene216_Q_2 | 72.9% | 140 | 87.0% | 50 | 0.0038 | 0.0128 |
| gene216_Q_1 | 89.4% | 132 | 95.8% | 48 | 0.0613 | 0.1924 |
| gene216_U_−1 | 86.1% | 140 | 93.0% | 50 | 0.0752 | 0.2087 |
| gene216_L_+1 | 87.0% | 138 | 94.0% | 50 | 0.0638 | 0.2367 |
| gene216_L_1 | 0.7% | 140 | 1.0% | 50 | 1.0000 | 1.0000 |
| gene216_L_−1 | 87.2% | 137 | 93.0% | 50 | 0.1400 | 0.3796 |
| gene216_L_−2 | 7.3% | 137 | 9.0% | 50 | 0.6623 | 0.5686 |
| gene216_V_+2 | 71.6% | 139 | 79.0% | 50 | 0.1860 | 0.3615 |
| gene216_V_+1 | 97.1% | 140 | 98.0% | 49 | 1.0000 | 1.0000 |
| gene216_I_1 | 83.7% | 138 | 91.0% | 50 | 0.0952 | 0.2406 |
| gene216_G_−1 | 9.9% | 137 | 10.2% | 49 | 1.0000 | 0.9269 |
| gene216_F_+1 | 64.1% | 128 | 73.3% | 43 | 0.1466 | 0.2885 |
| gene216_F_1 | 97.9% | 140 | 97.9% | 48 | 1.0000 | 1.0000 |
| gene216_D_1 | 0.0% | 139 | 1.0% | 50 | 0.2646 | 0.2646 |
| gene216_D_−2 | 0.7% | 139 | 1.0% | 49 | 1.0000 | 1.0000 |

TABLE 17
BHR
US population

| | Fre- | | | | | GENO- |
|---|---|---|---|---|---|---|
| | quencies | | | | ALLELE | TYPE |
| GENE_EXON | CNTL | N | CASE | N | P-VALUE | P-VALUE |
| gene216_T_2 | 67.8% | 76 | 46.4% | 14 | 0.0514 | 0.0409 |
| gene216_T_3 | 9.5% | 74 | 10.7% | 14 | 0.7369 | 1.0000 |
| gene216_T_4 | 94.7% | 75 | 100.0% | 13 | 0.6065 | 0.5986 |
| gene216_T_5 | 79.2% | 77 | 75.0% | 14 | 0.6206 | 0.6767 |
| gene216_T_6 | 76.6% | 77 | 75.0% | 14 | 0.8130 | 0.7738 |
| gene216_T_7 | 94.2% | 77 | 96.4% | 14 | 1.0000 | 1.0000 |
| gene216_T_8 | 93.9% | 74 | 96.4% | 14 | 1.0000 | 1.0000 |
| gene216_T_+1 | 82.9% | 76 | 78.6% | 14 | 0.5937 | 0.6635 |
| gene216_T_+2 | 36.3% | 73 | 32.1% | 14 | 0.8300 | 1.0000 |
| gene216_T_+4 | 23.0% | 76 | 28.6% | 14 | 0.6296 | 0.7242 |
| gene216_R_+2 | 89.6% | 77 | 96.4% | 14 | 0.4778 | 0.4545 |
| gene216_R_+1 | 92.0% | 69 | 76.9% | 13 | 0.0321 | 0.0452 |
| gene216_R_2 | 92.6% | 74 | 78.6% | 14 | 0.0333 | 0.0469 |
| gene216_R_1 | 7.8% | 77 | 28.6% | 14 | 0.0041 | 0.0072 |
| gene216_QR_+7 | 75.7% | 76 | 71.4% | 14 | 0.6391 | 0.2476 |
| gene216_QR_+6 | 98.7% | 77 | 100.0% | 14 | 1.0000 | 1.0000 |
| gene216_QR_+5 | 50.0% | 77 | 39.3% | 14 | 0.3130 | 0.4007 |
| gene216_QR_+4 | 57.1% | 77 | 64.3% | 14 | 0.5371 | 0.8691 |
| gene216_Q_+1 | 48.1% | 77 | 62.5% | 12 | 0.2724 | 0.4060 |
| gene216_Q_2 | 75.3% | 77 | 53.6% | 14 | 0.0233 | 0.0331 |
| gene216_Q_1 | 89.6% | 77 | 87.5% | 12 | 0.7250 | 0.5718 |
| gene216_U_−1 | 83.1% | 77 | 78.6% | 14 | 0.5910 | 0.6593 |
| gene216_L_+1 | 92.0% | 75 | 75.0% | 14 | 0.0149 | 0.0227 |
| gene216_L_1 | 0.6% | 77 | 0.0% | 14 | 1.0000 | 1.0000 |
| gene216_L_−1 | 92.0% | 75 | 75.0% | 14 | 0.0149 | 0.0227 |
| gene216_L_−2 | 6.7% | 75 | 7.1% | 14 | 1.0000 | 1.0000 |
| gene216_V_+2 | 70.8% | 77 | 60.7% | 14 | 0.3730 | 0.2711 |
| gene216_V_+1 | 94.2% | 77 | 96.4% | 14 | 1.0000 | 1.0000 |
| gene216_I_1 | 87.2% | 74 | 71.4% | 14 | 0.0455 | 0.0463 |
| gene216_G_−1 | 8.2% | 73 | 7.1% | 14 | 1.0000 | 1.0000 |
| gene216_F_+1 | 67.4% | 69 | 46.4% | 14 | 0.0510 | 0.0665 |
| gene216_F_1 | 94.8% | 77 | 96.4% | 14 | 1.0000 | 1.0000 |
| gene216_D_1 | 0.0% | 76 | 0.0% | 14 | 1.0000 | 1.0000 |
| gene216_D_−2 | 0.7% | 75 | 0.0% | 14 | 1.0000 | 1.0000 |

### EXAMPLE 13

### Haplotype Analyses

In addition to the analysis of individual SNPs, haplotype frequencies between the case and control groups were also compared. The haplotypes were constructed using a maximum likelihood approach. Since existing software for predicting haplotypes is unable to utilize individuals with missing data, a program was developed to make use of all individuals and, hence, provide more accurate haplotype frequency estimates. Haplotype analysis based on multiple SNPs in a gene is expected to provide increased evidence for an association between a given phenotype and that gene if all haplotyped SNPs are involved in the characterization of the phenotype. In other words, allelic variation involving those haplotyped SNPs are expected to be associated with different risks or susceptibilities toward the phenotype.

1. Asthma Phenotype:

The estimated frequency of each haplotype was compared between cases and controls by a permutation test. An overall comparison of the distribution of all haplotypes between the two groups was also performed. In Tables 18, 19 and 20 the haplotype analysis (2-at-a-time) for all SNPs in Gene 216 is presented for the combined, the UK and the US populations, respectively. The diagonal entries represent the single SNP p-values, while the other entries are the p-values for a test of association between the asthma phenotype and the haplotypes defined by the 2 SNPs listed on the horizontal and vertical axes. The frequency of the individual SNPs in the cases and controls are shown at the bottom of the tables. Colored cells indicate p-values that were statistically significant (light gray: 0.01 to 0.05, dark gray: 0.001 to 0.0099, black: <0.001). As seen in Table 18, haplotypes defined by SNPs T5 & T8, SNPs T+2 & QR+4, T5 & T7 and SNPs T4 & T5, yielded highly significant p-values of 0.00039, 0.000042, 0.00056 and 0.00042 respectively, which were more significant than the analysis of these SNPs alone (T4 p=0.16; T5 p=0.04; T7 p=0.16; T8 p=0.25; T+2 p=0.68; QR+4 p=0.04). These associations were also more significant than the one observed for the single SNP T+1 reported above. In the UK population, the most significant association was found in Gene 216 (Table 19) with five haplotypes significant at the 0.001 level (SNPs T+2 & QR+4, p=0.000021; QR+5 & QR+4, p=0.00051; QR+4 & Q+1 p=0.00066; QR+6 & Q2, p=0.00062; and QR+4 & Q2, p=0.00023). Forty four haplotypes were significant at the 0.01 level in Gene 216 (Table 19) in the UK population. In the US population, numerous haplotypes were significant at the 0.01 level for Genes 216 (Table 20).

[image]

[image]

[image]

[image]

[image]

[image]

2. Bronchial Hyper-Responsiveness:

A similar test for association of 2-SNP-a-time haplotypes with BHR(PC₂₀≦16 mg/ml) was performed. In Tables 21, 22 and 23, the haplotype analysis (2-at-a-time) for all SNPs in Gene 216 is presented for the combined, the UK and the US populations, respectively. One haplotype in Gene 216 (Table 21: SNPs T+2 & QR+4, p=0.0041) was significant at the 0.01 level in the combined sample. In contrast, in the UK population, seventeen haplotypes were significant at the 0.01 level in Gene 216 (Table 22). In the US population, nine haplotypes were significant at the 0.01 level in Gene 216 (Table 23). Tables 18, 19, and 20 and Tables 21, 22 and 23 showed similar patterns of significance with lower level achieved in the BHR analysis due to the reduced sample size in the (PC₂₀≦16 mg/ml) subgroup.

In summary, haplotype analysis of SNPs significantly strengthened the evidence in support of Gene 216 as an asthma susceptibility gene. In some SNP combinations, the association was increased by an order of magnitude. The most striking association again appeared in the 3′ region of the gene, in agreement with the single SNP analysis.

TABLE 21

| | 216_T_2 | 216_T_3 | 216_T_4 | 216_T_5 | 216_T_6 | 216_T_7 | 216_T_8 | |
|---|---|---|---|---|---|---|---|---|
| 216_T_2 | 0.8294 | 0.8699 | 0.6528 | 0.9001 | 0.6497 | 0.8157 | 0.8569 | 216_T_2 |
| 216_T_3 | — | 0.8592 | 0.4838 | 0.7833 | 0.8337 | 0.6421 | 0.6733 | 216_T_3 |
| 216_T_4 | — | — | 0.3878 | 0.0801 | 0.6596 | 0.6816 | 0.6683 | 216_T_4 |
| 216_T_5 | — | — | — | 0.5428 | 0.5556 | 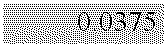 | 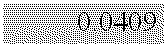 | 216_T_5 |
| 216_T_6 | — | — | — | — | 1 | 0.7439 | 0.7536 | 216_T_6 |
| 216_T_7 | — | — | — | — | — | 0.5856 | 0.3561 | 216_T_7 |
| 216_T_8 | — | — | — | — | — | — | 0.7758 | 216_T_8 |
| 216_T_+1 | — | — | — | — | — | — | — | 216_T_+1 |
| 216_T_+2 | — | — | — | — | — | — | — | 216_T_+2 |
| 216_T_+4 | — | — | — | — | — | — | — | 216_T_+4 |
| 216_R_+2 | — | — | — | — | — | — | — | 216_R_+2 |
| 216_R_+1 | — | — | — | — | — | — | — | 216_R_+1 |
| 216_R_2 | — | — | — | — | — | — | — | 216_R_2 |
| 216_R_1 | — | — | — | — | — | — | — | 216_R_1 |
| 216_QR_+7 | — | — | — | — | — | — | — | 216_QR_+7 |
| 216_QR_+6 | — | — | — | — | — | — | — | 216_QR_+6 |
| 216_QR_+5 | — | — | — | — | — | — | — | 216_QR_+5 |
| 216_QR_+4 | — | — | — | — | — | — | — | 216_QR_+4 |
| 216_Q_+1 | — | — | — | — | — | — | — | 216_Q_+1 |
| 216_Q_2 | — | — | — | — | — | — | — | 216_Q_2 |
| 216_Q_1 | — | — | — | — | — | — | — | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 66.50% | 8.70% | 96.30% | 76.70% | 77.80% | 96.30% | 96.50% | CNTL |
| CASE | 67.70% | 9.40% | 98.40% | 79.80% | 78.30% | 97.70% | 97.60% | CASE |
| | 216_T_+1 | 216_T_+2 | 216_T_+4 | 216_R_+2 | 216_R_+1 | 216_R_2 | 216_R_1 | |
| 216_T_2 | 0.3137 | 0.6102 | 0.8654 | 0.9227 | 0.8548 | 0.9513 | 0.9968 | 216_T_2 |
| 216_T_3 | 0.2704 | 0.4217 | 0.5951 | 0.9937 | 0.71 | 0.8294 | 0.9975 | 216_T_3 |
| 216_T_4 | 0.4037 | 0.426 | 0.474 | 0.2036 | 0.4754 | 0.4846 | 0.4814 | 216_T_4 |
| 216_T_5 | 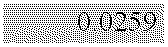 | 0.6817 | 0.7754 | 0.7406 | 0.8656 | 0.7605 | 0.8568 | 216_T_5 |
| 216_T_6 | 0.3846 | 0.7367 | 0.6802 | 0.866 | 0.8322 | 0.9808 | 0.9949 | 216_T_6 |
| 216_T_7 | 0.264 | 0.5599 | 0.7258 | 0.4951 | 0.7442 | 0.746 | 0.7652 | 216_T_7 |
| 216_T_8 | 0.2735 | 0.614 | 0.7615 | 0.5474 | 0.7837 | 0.7875 | 0.795 | 216_T_8 |
| 216_T_+1 | 0.1413 | 0.4229 | 0.2936 | 0.1518 | 0.1374 | 0.2295 | 0.1848 | 216_T_+1 |
| 216_T_+2 | — | 0.3978 | 0.8361 | 0.6592 | 0.1325 | 0.3537 | 0.3367 | 216_T_+2 |
| 216_T_+4 | — | — | 0.6421 | 0.9199 | 0.6672 | 0.8959 | 0.8684 | 216_T_+4 |
| 216_R_+2 | — | — | — | 1 | 0.8319 | 0.9837 | 0.9783 | 216_R_+2 |
| 216_R_+1 | — | — | — | — | 1 | 0.8591 | 0.5707 | 216_R_+1 |
| 216_R_2 | — | — | — | — | — | 1 | 0.338 | 216_R_2 |
| 216_R_1 | — | — | — | — | — | — | 0.875 | 216_R_1 |
| 216_QR_+7 | — | — | — | — | — | — | — | 216_QR_+7 |
| 216_QR_+6 | — | — | — | — | — | — | — | 216_QR_+6 |
| 216_QR_+5 | — | — | — | — | — | — | — | 216_QR_+5 |
| 216_QR_+4 | — | — | — | — | — | — | — | 216_QR_+4 |
| 216_Q_+1 | — | — | — | — | — | — | — | 216_Q_+1 |
| 216_Q_2 | — | — | — | — | — | — | — | 216_Q_2 |
| 216_Q_1 | — | — | — | — | — | — | — | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 85.20% | 37.30% | 24.40% | 88.30% | 88.70% | 90.60% | 11.30% | CNTL |
| CASE | 90.60% | 41.80% | 26.60% | 88.30% | 89.20% | 91.10% | 11.70% | CASE |
| | 216_QR_+7 | 216_QR_+6 | 216_QR_+5 | 216_QR_+4 | 216_Q_+1 | 216_Q_2 | 216_Q_1 | |
| 216_T_2 | 0.6127 | 0.809 | 0.4618 | 0.3714 | 0.2027 | 0.4985 | 0.5787 | 216_T_2 |
| 216_T_3 | 0.6953 | 0.8966 | 0.9682 | 0.5282 | 0.9662 | 0.6224 | 0.1274 | 216_T_3 |
| 216_T_4 | 0.6119 | 0.2507 | 0.6758 | 0.372 | 0.5948 | 0.4522 | 0.1688 | 216_T_4 |
| 216_T_5 | 0.7124 | 0.6804 | 0.6199 | 0.2383 | 0.7883 | 0.5364 | 0.4421 | 216_T_5 |
| 216_T_6 | 0.5552 | 0.8049 | 0.937 | 0.2731 | 0.8839 | 0.431 | 0.2757 | 216_T_6 |
| 216_T_7 | 0.5427 | 0.4354 | 0.8044 | 0.33 | 0.8655 | 0.374 | 0.2692 | 216_T_7 |
| 216_T_8 | 0.5818 | 0.4673 | 0.8358 | 0.3389 | 0.901 | 0.3728 | 0.3041 | 216_T_8 |
| 216_T_+1 | 0.2476 | 0.2972 | 0.2684 | 0.1303 | 0.349 | 0.2692 | 0.2569 | 216_T_+1 |
| 216_T_+2 | 0.1168 | 0.4177 | 0.8063 | 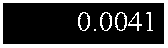 | 0.6387 | 0.6177 | 0.3046 | 216_T_+2 |
| 216_T_+4 | 0.4637 | 0.6411 | 0.827 | 0.0709 | 0.7037 | 0.5357 | 0.3112 | 216_T_+4 |
| 216_R_+2 | 0.4454 | 0.8445 | 0.9937 | 0.3199 | 0.9869 | 0.3483 | 0.1773 | 216_R_+2 |
| 216_R_+1 | 0.6045 | 0.7804 | 0.9553 | 0.0906 | 0.8743 | 0.5514 | 0.1027 | 216_R_+1 |
| 216_R_2 | 0.6225 | 0.7814 | 0.977 | 0.208 | 0.9485 | 0.4841 | 0.1249 | 216_R_2 |
| 216_R_1 | 0.6402 | 0.8153 | 0.3556 | 0.197 | 0.3474 | 0.2054 | 0.1515 | 216_R_1 |
| 216_QR_+7 | 0.3876 | 0.3756 | 0.0564 | 0.1251 | 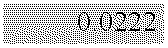 | 0.497 | 0.3984 | 216_QR_+6 |
| 216_QR_+6 | — | 1 | 0.8309 | 0.21 | 0.8314 | 0.3909 | 0.3646 | 216_QR_+4 |
| 216_QR_+5 | — | — | 1 | 0.0265 | 0.9982 | 0.3183 | 0.2453 | 216_Q_+1 |
| 216_QR_+4 | — | — | — | 0.1521 | 0.0611 | 0.1626 | 0.1368 | 216_Q_2 |
| 216_Q_+1 | — | — | — | — | 1 | 0.2097 | 0.2725 | 216_Q_1 |
| 216_Q_2 | — | — | — | — | — | 0.2009 | 0.3331 | 216_U_−1 |
| 216_Q_1 | — | — | — | — | — | — | 0.1565 | 216_L_+1 |
| 216_U_−1 | — | — | — | — | — | — | — | 216_L_1 |
| 216_L_+1 | — | — | — | — | — | — | — | 216_L_−1 |
| 216_L_1 | — | — | — | — | — | — | — | 216_L_−2 |
| 216_L_−1 | — | — | — | — | — | — | — | 216_V_+2 |
| 216_L_−2 | — | — | — | — | — | — | — | 216_V_+1 |
| 216_V_+2 | — | — | — | — | — | — | — | 216_I_1 |
| 216_V_+1 | — | — | — | — | — | — | — | 216_G_−1 |
| 216_I_1 | — | — | — | — | — | — | — | 216_F_+1 |
| 216_G_−1 | — | — | — | — | — | — | — | 216_F_1 |
| 216_F_+1 | — | — | — | — | — | — | — | 216_D_1 |
| 216_F_1 | — | — | — | — | — | — | — | 216_D_−2 |
| 216_D_1 | — | — | — | — | — | — | — | |
| 216_D_−2 | — | — | — | — | — | — | — | |
| CNTL | 78.10% | 99.50% | 46.40% | 51.50% | 51.20% | 73.70% | 89.50% | CNTL |
| CASE | 82.00% | 100.00% | 46.80% | 58.90% | 51.80% | 79.70% | 94.20% | CASE |
| | 216_U_−1 | 216_L_+1 | 216_L_1 | 216_L_−1 | 216_L_−2 | 216_V_+2 | 216_V_+1 | |
| 216_T_2 | 0.6007 | 0.9369 | 0.9292 | 0.9947 | 0.7441 | 0.8759 | 0.7461 | 216_T_2 |
| 216_T_3 | 0.3869 | 0.9807 | 0.92266 | 0.9835 | 0.6939 | 0.6655 | 0.5735 | 216_T_3 |
| 216_T_4 | 0.4878 | 0.4333 | 0.6387 | 0.4707 | 0.5001 | 0.3521 | 0.6522 | 216_T_4 |
| 216_T_5 | 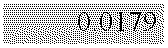 | 0.8419 | 0.8272 | 0.8799 | 0.7291 | 0.5465 | 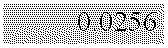 | 216_T_5 |
| 216_T_6 | 0.5172 | 0.9373 | 0.3839 | 0.9918 | 0.8763 | 0.6743 | 0.7197 | 216_T_6 |
| 216_T_7 | 0.3785 | 0.6817 | 0.774 | 0.7393 | 0.7235 | 0.6851 | 0.7076 | 216_T_7 |
| 216_T_8 | 0.3782 | 0.7262 | 0.7647 | 0.7922 | 0.7622 | 0.7673 | 0.6937 | 216_T_8 |
| 216_T_+1 | 0.1884 | 0.1217 | 0.2688 | 0.1549 | 0.2174 | 0.3823 | 0.3985 | 216_T_+1 |
| 216_T_+2 | 0.4568 | 04714 | 0.7192 | 0.419 | 0.3029 | 0.2318 | 0.5451 | 216_T_+2 |
| 216_T_+4 | 0.5279 | 0.8787 | 0.477 | 0.8913 | 0.6089 | 0.4624 | 0.5545 | 216_T_+4 |
| 216_R_+2 | 0.0904 | 0.9371 | 0.9874 | 0.9912 | 0.9466 | 0.694 | 0.5058 | 216_R_+2 |
| 216_R_+1 | 0.2215 | 0.8563 | 0.9592 | 0.9952 | 0.8919 | 0.8423 | 0.6891 | 216_R_+1 |
| 216_R_2 | 0.3228 | 0.8735 | 0.9512 | 0.5874 | 0.8544 | 0.6935 | 0.7084 | 216_R_2 |
| 216_R_1 | 0.2889 | 0.2987 | 0.9506 | 0.2671 | 0.8668 | 0.7229 | 0.7163 | 216_R_1 |
| 216_QR_+7 | 0.3418 | 0.5129 | 0.724 | 0.6153 | 0.3508 | 0.6796 | 0.6562 | 216_QR_+7 |
| 216_QR_+6 | 0.5159 | 0.6427 | 0.8251 | 0.8337 | 0.7354 | 0.6436 | 0.3846 | 216_QR_+6 |
| 216_QR_+5 | 0.185 | 0.9356 | 0.977 | 0.3226 | 0.8535 | 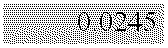 | 0.7561 | 216_QR_+5 |
| 216_QR_+4 | 0.1257 | 0.1971 | 0.2895 | 0.2002 | 0.2075 | 0.0632 | 0.3431 | 216_QR_+4 |
| 216_Q_+1 | 0.3284 | 0.8919 | 0.9458 | 0.3135 | 0.8539 | 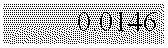 | 0.8169 | 216_Q_+1 |
| 216_Q_2 | 0.3349 | 0.3249 | 0.5627 | 0.1721 | 0.3748 | 0.3227 | 0.4849 | 216_Q_2 |
| 216_Q_1 | 0.3747 | 0.1173 | 0.3107 | 0.1415 | 0.1125 | 0.268 | 0.1866 | 216_Q_1 |
| 216_U_−1 | 0.1915 | 0.1873 | 0.5405 | 0.2376 | 0.3173 | 0.3582 | 0.509 | 216_U_−1 |
| 216_L_+1 | — | 0.8722 | 0.8927 | 0.4469 | 0.8646 | 0.6223 | 0.6449 | 216_L_+1 |
| 216_L_1 | — | — | 1 | 0.9676 | 0.8249 | 0.2583 | 0.7503 | 216_L_1 |
| 216_L_−1 | — | — | — | 1 | 0.9027 | 0.6942 | 0.7054 | 216_L_−1 |
| 216_L_−2 | — | — | — | — | 0.5661 | 0.5297 | 0.6783 | 216_L_−2 |
| 216_V_+2 | — | — | — | — | — | 0.4343 | 0.6819 | 216_V_+2 |
| 216_V_+1 | — | — | — | — | — | — | 0.5874 | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 85.00% | 88.70% | 0.70% | 88.90% | 7.10% | 71.30% | 96.10% | CNTL |
| CASE | 89.80% | 89.80% | 0.80% | 89.10% | 8.60% | 75.00% | 97.60% | CASE |

| | 216_I_1 | 216_G_−1 | 216_F_+1 | 216_F_1 | 216_D_1 | 216_D_−2 | |
|---|---|---|---|---|---|---|---|
| 216_T_2 | 0.9473 | 0.9753 | 0.9665 | 0.9031 | 0.2822 | 0.9148 | 216_T_2 |
| 216_T_3 | 0.8079 | 0.8659 | 0.894 | 0.732 | 0.2896 | 0.9415 | 216_T_3 |
| 216_T_4 | 0.6115 | 0.6035 | 0.6912 | 0.5512 | 0.1272 | 0.6373 | 216_T_4 |
| 216_T_5 | 0.3726 | 0.9338 | 0.8212 | 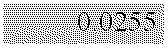 | 0.1684 | 0.7477 | 216_T_5 |
| 216_T_6 | 0.4388 | 0.9627 | 0.6782 | 0.9025 | 0.3121 | 0.9493 | 216_T_6 |
| 216_T_7 | 0.8106 | 0.8494 | 0.749 | 0.4337 | 0.1823 | 0.7671 | 216_T_7 |
| 216_T_8 | 0.8463 | 0.9027 | 0.7612 | 0.539 | 0.1647 | 0.7718 | 216_T_8 |
| 216_T_+1 | 0.1619 | 0.4265 | 0.4195 | 0.2726 | 0.0542 | 0.2739 | 216_T_+1 |
| 216_T_+2 | 0.8303 | 0.1377 | 0.7163 | 0.6594 | 0.1451 | 0.7493 | 216_T_+2 |
| 216_T_+4 | 0.7345 | 0.2519 | 0.8486 | 0.8646 | 0.2115 | 0.8406 | 216_T_+4 |
| 216_R_+2 | 0.8759 | 0.999 | 0.9922 | 0.6347 | 0.3025 | 0.9783 | 216_R_+2 |
| 216_R_+1 | 0.7137 | 0.8918 | 0.9842 | 0.9141 | 0.3019 | 0.97834 | 216_R_+1 |
| 216_R_2 | 0.6623 | 0.8538 | 0.7704 | 0.9203 | 0.3276 | 0.9797 | 216_R_2 |
| 216_R_1 | 0.7775 | 0.9891 | 0.9096 | 0.9257 | 0.2969 | 0.94 | 216_R_1 |
| 216_QR_+7 | 0.5829 | 0.5411 | 0.722 | 0.6178 | 0.1341 | 0.7947 | 216_QR_+7 |
| 216_QR_+6 | 0.54 | 0.9664 | 0.7774 | 0.6443 | 0.1566 | 0.81 | 216_QR_+6 |
| 216_QR_+5 | 0.9466 | 0.9434 | 0.5968 | 0.9188 | 0.3188 | 0.9019 | 216_QR_+5 |
| 216_QR_+4 | 0.3571 | 0.2455 | 0.4052 | 0.355 | 0.087 | 0.4236 | 216_QR_+4 |
| 216_Q_+1 | 0.9418 | 0.6496 | 0.2272 | 0.9075 | 0.2945 | 0.8972 | 216_Q_+1 |
| 216_Q_2 | 0.4143 | 0.5794 | 0.2689 | 0.3845 | 0.0808 | 0.6666 | 216_Q_2 |
| 216_Q_1 | 0.135 | 0.3023 | 0.3737 | 0.2772 | 0.0535 | 0.311 | 216_Q_1 |
| 216_U_−1 | 0.1412 | 0.6101 | 0.5283 | 0.3593 | 0.0715 | 0.5417 | 216_U_−1 |
| 216_L_+1 | 0.7264 | 0.9664 | 0.6497 | 0.8604 | 0.2819 | 0.8878 | 216_L_+1 |
| 216_L_1 | 0.841 | 0.9814 | 0.9135 | 0.8975 | 0.1975 | 0.8815 | 216_L_1 |
| 216_L_−1 | 0.8977 | 0.9983 | 0.9637 | 0.9181 | 0.3408 | 0.6135 | 216_L_−1 |
| 216_L_−2 | 0.8107 | 0.7618 | 0.776 | 0.8166 | 0.2206 | 0.8115 | 216_L_−2 |
| 216_V_+2 | 0.7195 | 0.7148 | 0.8358 | 0.8174 | 0.1638 | 0.7397 | 216_V_+2 |
| 216_V_+1 | 0.8032 | 0.7974 | 0.7905 | 0.5318 | 0.1413 | 0.7527 | 216_V_+1 |
| 216_I_1 | 0.6709 | 0.8919 | 0.7447 | 0.8389 | 0.2255 | 0.8588 | 216_I_1 |
| 216_G_−1 | — | 1 | 0.6949 | 0.9566 | 0.3243 | 0.9938 | 216_G_−1 |
| 216_F_+1 | — | — | 0.8234 | 0.8619 | 0.2797 | 0.9023 | 216_F_+1 |
| 216_F_1 | — | — | — | 0.7752 | 0.2845 | 0.902 | 216_F_1 |
| 216_D_1 | — | — | — | — | 0.2294 | 0.2139 | 216_D_1 |
| 216_D_−2 | — | — | — | — | — | 1 | 216_D_−2 |
| CNTL | 84.90% | 9.30% | 65.20% | 96.80% | 0.00% | 0.70% | CNTL |
| CASE | 86.70% | 9.50% | 66.70% | 97.60% | 0.80% | 0.80% | CASE |

TABLE 22

| | 216_T_2 | 216_T_3 | 216_T_4 | 216_T_5 | 216_T_6 | 216_T_7 | 216_T_8 | |
|---|---|---|---|---|---|---|---|---|
| 216_T_2 | 0.1635 | 0.1946 | 0.4642 | 0.2542 | 0.2534 | 0.4757 | 0.4674 | 216_T_2 |
| 216_T_3 | — | 0.8352 | 0.9346 | 0.5085 | 0.9737 | 0.9445 | 0.9524 | 216_T_3 |
| 216_T_4 | — | — | 1 | 0.1832 | 0.891 | 0.7313 | 0.4727 | 216_T_4 |
| 216_T_5 | — | — | — | 0.2641 | 0.2902 | 0.221 | 0.2228 | 216_T_5 |
| 216_T_6 | — | — | — | — | 1 | 0.9555 | 0.9371 | 216_T_6 |
| 216_T_7 | — | — | — | — | — | 1 | 0.7135 | 216_T_7 |
| 216_T_8 | — | — | — | — | — | — | 1 | 216_T_8 |
| 216_T_+1 | — | — | — | — | — | — | — | 216_T_+1 |
| 216_T_+2 | — | — | — | — | — | — | — | 216_T_+2 |
| 216_T_+4 | — | — | — | — | — | — | — | 216_T_+4 |
| 216_R_+2 | — | — | — | — | — | — | — | 216_R_+2 |
| 216_R_+1 | — | — | — | — | — | — | — | 216_R_+1 |
| 216_R_2 | — | — | — | — | — | — | — | 216_R_2 |
| 216_R_1 | — | — | — | — | — | — | — | 216_R_1 |
| 216_QR_+7 | — | — | — | — | — | — | — | 216_QR_+7 |
| 216_QR_+6 | — | — | — | — | — | — | — | 216_QR_+6 |
| 216_QR_+5 | — | — | — | — | — | — | — | 216_QR_+5 |
| 216_QR_+4 | — | — | — | — | — | — | — | 216_QR_+4 |
| 216_Q_+1 | — | — | — | — | — | — | — | 216_Q_+1 |
| 216_Q_2 | — | — | — | — | — | — | — | 216_Q_2 |
| 216_Q_1 | — | — | — | — | — | — | — | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 65.80% | 8.30% | 97.10% | 75.40% | 78.50% | 97.50% | 97.80% | CNTL |
| CASE | 74.00% | 9.00% | 98.00% | 81.30% | 79.40% | 98.00% | 98.00% | CASE |
| | 216_T_+1 | 216_T_+2 | 216_T_+4 | 216_R_+2 | 216_R_+1 | 216_R_2 | 216_R_1 | |
| 216_T_2 | 0.1699 | 0.0577 | 0.2983 | 0.3236 | 0.2864 | 0.1937 | 0.2868 | 216_T_2 |
| 216_T_3 | 0.1119 | 0.2317 | 0.5889 | 0.8441 | 0.4145 | 0.2901 | 0.4045 | 216_T_3 |
| 216_T_4 | 0.1102 | 0.6516 | 0.6303 | 0.5016 | 0.279 | 0.2638 | 0.2006 | 216_T_4 |
| 216_T_5 | 0.0834 | 0.3727 | 0.415 | 0.2866 | 0.1992 | 0.132 | 0.15 | 216_T_5 |
| 216_T_6 | 0.2145 | 0.6693 | 0.4986 | 0.6354 | 0.3337 | 0.3054 | 0.2174 | 216_T_6 |
| 216_T_7 | 0.105 | 0.6713 | 0.7568 | 0.5668 | 0.277 | 0.2847 | 0.235 | 216_T_7 |
| 216_T_8 | 0.0868 | 0.6249 | 0.8635 | 0.584 | 0.3144 | 0.3013 | 0.2432 | 216_T_8 |
| 216_T_+1 | 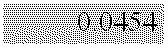 | 0.0896 | 0.1191 | 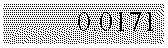 | 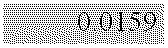 | 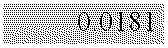 | 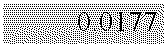 | 216_T_+1 |
| 216_T_+2 | — | 0.2715 | 0.6143 | 0.6109 | 0.1093 | 0.1696 | 0.1672 | 216_T_+2 |
| 216_T_+4 | — | — | 0.8938 | 0.8941 | 0.4271 | 0.3497 | 0.2603 | 216_T_+4 |
| 216_R_+2 | — | — | — | 0.729 | 0.2922 | 0.3083 | 0.2672 | 216_R_+2 |
| 216_R_+1 | — | — | — | — | 0.1838 | 0.3156 | 0.2629 | 216_R_+1 |
| 216_R_2 | — | — | — | — | — | 0.1494 | 0.1886 | 216_R_2 |
| 216_R_1 | — | — | — | — | — | — | 0.1041 | 216_R_1 |
| 216_QR_+7 | — | — | — | — | — | — | — | 216_QR_+7 |
| 216_QR_+6 | — | — | — | — | — | — | — | 216_QR_+6 |
| 216_QR_+5 | — | — | — | — | — | — | — | 216_QR_+5 |
| 216_QR_+4 | — | — | — | — | — | — | — | 216_QR_+4 |
| 216_Q_+1 | — | — | — | — | — | — | — | 216_Q_+1 |
| 216_Q_2 | — | — | — | — | — | — | — | 216_Q_2 |
| 216_Q_1 | — | — | — | — | — | — | — | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 86.40% | 37.90% | 25.20% | 87.50% | 86.90% | 89.60% | 13.20% | CNTL |
| CASE | 94.00% | 44.70% | 26.00% | 86.00% | 92.60% | 94.80% | 7.00% | CASE |
| | 216_QR_+7 | 216_QR_+6 | 216_QR_+5 | 216_QR_+4 | 216_Q_+1 | 216_Q_2 | 216_Q_1 | |
| 216_T_2 | 0.41 | 0.151 | 0.0598 | 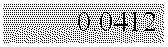 | 0.0993 | 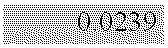 | 0.1363 | 216_T_2 |
| 216_T_3 | 0.0868 | 0.8146 | 0.7043 | 0.1865 | 0.6845 | 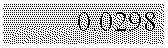 | 0.1044 | 216_T_3 |
| 216_T_4 | 0.4975 | 0.7313 | 0.7734 | 0.3353 | 0.8254 | 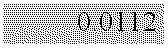 | 0.1712 | 216_T_4 |
| 216_T_5 | 0.3394 | 0.2043 | 0.1396 | 0.0658 | 0.3609 | 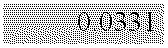 | 0.1884 | 216_T_5 |
| 216_T_6 | 0.4571 | 0.846 | 0.7353 | 0.2584 | 0.8496 | 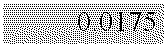 | 0.1382 | 216_T_6 |
| 216_T_7 | 0.4779 | 0.8161 | 0.7714 | 0.3188 | 0.8564 | 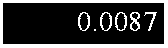 | 0.2165 | 216_T_7 |
| 216_T_8 | 0.4423 | 0.8072 | 0.8158 | 0.3266 | 0.8823 | 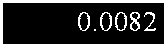 | 0.2404 | 216_T_8 |
| 216_T_+1 | 0.0993 | 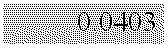 | 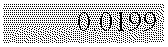 | 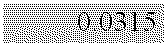 | 0.0873 | 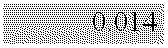 | 0.1085 | 216_T_+1 |
| 216_T_+2 | 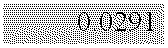 | 0.2774 | 0.0937 | 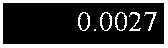 | 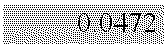 | 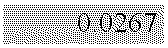 | 0.1205 | 216_T_+2 |
| 216_T_+4 | 0.3809 | 0.8728 | 0.5997 | 0.1248 | 0.4254 | 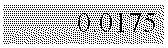 | 0.1491 | 216_T_+4 |
| 216_R_+2 | 0.3422 | 0.6525 | 0.5864 | 0.1828 | 0.6558 | 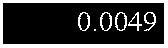 | 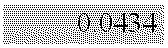 | 216_R_+2 |
| 216_R_+1 | 0.1076 | 0.1376 | 0.2608 | 0.0465 | 0.2608 | 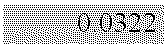 | 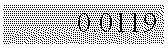 | 216_R_+1 |
| 216_R_2 | 0.1264 | 0.1401 | 0.2309 | 0.0603 | 0.2443 | 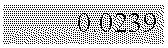 | 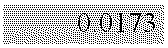 | 216_R_2 |
| 216_R_1 | 0.0702 | 0.1153 | 0.2645 | 0.0454 | 0.2559 | 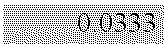 | 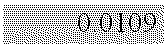 | 216_R_1 |
| 216_QR_+7 | 0.2983 | 0.2237 | 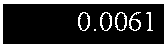 | 0.0362 | 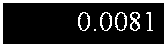 | 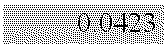 | 0.1453 | 216_QR_+7 |
| 216_QR_+6 | — | 1 | 0.4576 | 0.16 | 0.55 | 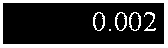 | 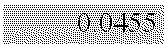 | 216_QR_+6 |
| 216_QR_+5 | — | — | 0.4771 | 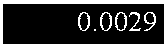 | 0.8707 | 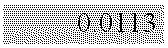 | 0.0561 | 216_QR_+5 |
| 216_QR_+4 | — | — | — | 0.1508 | 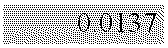 | 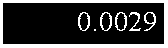 | 0.0531 | 216_QR_+4 |
| 216_Q_+1 | — | — | — | — | 0.5407 | 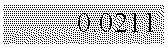 | 0.0536 | 216_Q_+1 |
| 216_Q_2 | — | — | — | — | — | 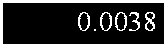 | 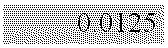 | 216_Q_2 |
| 216_Q_1 | — | — | — | — | — | — | 0.0613 | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 79.50% | 0.00% | 44.40% | 48.10% | 53.10% | 72.90% | 89.40% | CNTL |
| CASE | 85.00% | 0.00% | 49.00% | 57.30% | 48.90% | 87.00% | 95.80% | CASE |
| | 216_U_−1 | 216_L_+1 | 216_L_1 | 216_L_−1 | 216_L_−2 | 216_V_+2 | 216_V_+1 | |
| 216_T_2 | 0.2354 | 0.2108 | 0.4205 | 0.2791 | 0.1304 | 0.2571 | 0.4636 | 216_T_2 |
| 216_T_3 | 0.1847 | 0.2611 | 0.9291 | 0.4768 | 0.4817 | 0.202 | 0.9277 | 216_T_3 |
| 216_T_4 | 0.1539 | 0.1427 | 0.8569 | 0.2395 | 0.7921 | 0.3405 | 0.7155 | 216_T_4 |
| 216_T_5 | 0.0872 | 0.097 | 0.7714 | 0.1855 | 0.507 | 0.3068 | 0.1748 | 216_T_5 |
| 216_T_6 | 0.3072 | 0.1424 | 0.4052 | 0.2802 | 0.9045 | 0.3762 | 0.9218 | 216_T_6 |
| 216_T_7 | 0.1593 | 0.1465 | 0.9358 | 0.2551 | 0.8878 | 0.3588 | 0.7347 | 216_T_7 |
| 216_T_8 | 0.1703 | 0.1848 | 0.9092 | 0.2773 | 0.9105 | 0.4628 | 0.476 | 216_T_8 |
| 216_T_+1 | 0.1081 | 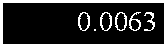 | 0.0966 | 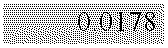 | 0.1105 | 0.108 | 0.1116 | 216_T_+1 |
| 216_T_+2 | 0.1096 | 0.1576 | 0.6858 | 0.2164 | 0.4992 | 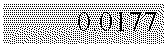 | 0.6495 | 216_T_+2 |
| 216_T_+4 | 0.2888 | 0.1708 | 0.6009 | 0.3196 | 0.8842 | 0.1628 | 0.6625 | 216_T_+4 |
| 216_R_+2 | 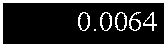 | 0.1525 | 0.9209 | 0.2989 | 0.7197 | 0.3294 | 0.5441 | 216_R_+2 |
| 216_R_+1 | 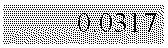 | 0.2472 | 0.362 | 0.6439 | 0.4755 | 0.1176 | 0.2779 | 216_R_+1 |
| 216_R_2 | 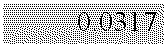 | 0.2588 | 0.3122 | 0.2887 | 0.3647 | 0.0767 | 0.2614 | 216_R_2 |
| 216_R_1 | 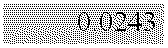 | 0.1321 | 0.2283 | 0.1027 | 0.2925 | 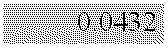 | 0.2021 | 216_R_1 |
| 216_QR_+7 | 0.18 | 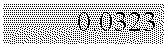 | 0.6684 | 0.0796 | 0.125 | 0.3654 | 0.5079 | 216_QR_+7 |
| 216_QR_+6 | 0.0663 | 0.0568 | 0.8893 | 0.1137 | 0.65 | 0.1144 | 0.7358 | 216_QR_+6 |
| 216_QR_+5 | 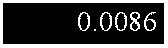 | 0.1599 | 0.7718 | 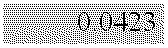 | 0.6116 | 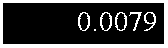 | 0.7782 | 216_QR_+5 |
| 216_QR_+4 | 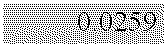 | 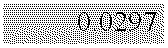 | 0.2615 | 0.0558 | 0.1816 | 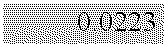 | 0.3334 | 216_QR_+4 |
| 216_Q_+1 | 0.052 | 0.1512 | 0.7981 | 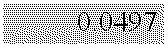 | 0.5841 | 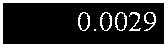 | 0.8282 | 216_Q_+1 |
| 216_Q_2 | 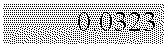 | 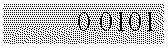 | 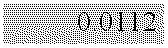 | 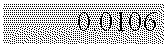 | 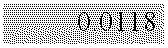 | 0.0098 | 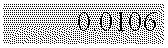 | 216_Q_2 |
| 216_Q_1 | 0.1043 | 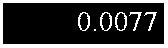 | 0.1176 | 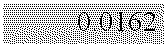 | 0.1069 | 0.094 | 0.1753 | 216_Q_1 |
| 216_U_−1 | 0.0752 | 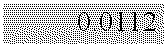 | 0.1367 | 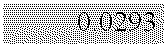 | 0.1799 | 0.0683 | 0.1592 | 216_U_−1 |
| 216_L_+1 | — | 0.0638 | 0.1326 | 0.0572 | 0.1554 | 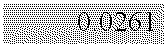 | 0.1422 | 216_L_+1 |
| 216_L_1 | — | — | 1 | 0.2934 | 0.8458 | 0.1022 | 0.8558 | 216_L_1 |
| 216_L_−1 | — | — | — | 0.014 | 0.3116 | 0.0638 | 0.2334 | 216_L_−1 |
| 216_L_−2 | — | — | — | — | 0.6623 | 0.2018 | 0.7842 | 216_L_−2 |
| 216_V_+2 | — | — | — | — | — | 0.186 | 0.3583 | 216_V_+2 |
| 216_V_+1 | — | — | — | — | — | — | 1 | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 86.10% | 87.00% | 0.70% | 87.20% | 7.30% | 71.60% | 97.10% | CNTL |
| CASE | 93.00% | 94.00% | 1.00% | 93.00% | 9.00% | 79.00% | 98.00% | CASE |

| | 216_I_1 | 216_G_−1 | 216_F_+1 | 216_F_1 | 216_D_1 | 216_D_−2 | |
|---|---|---|---|---|---|---|---|
| 216_T_2 | 0.3122 | 0.4106 | 0.2736 | 0.4814 | 0.0819 | 0.3287 | 216_T_2 |
| 216_T_3 | 0.3057 | 0.6908 | 0.1932 | 0.9356 | 0.3363 | 0.8904 | 216_T_3 |
| 216_T_4 | 0.1905 | 0.8917 | 0.274 | 0.733 | 0.2708 | 0.8707 | 216_T_4 |
| 216_T_5 | 0.1044 | 0.6102 | 0.3101 | 0.1393 | 0.1039 | 0.6496 | 216_T_5 |
| 216_T_6 | 0.2911 | 0.9952 | 0.2941 | 0.9375 | 0.3535 | 0.9013 | 216_T_6 |
| 216_T_7 | 0.1891 | 0.9602 | 0.2734 | 0.6747 | 0.3149 | 0.8817 | 216_T_7 |
| 216_T_8 | 0.1876 | 0.9762 | 0.2664 | 0.8999 | 0.3484 | 0.9021 | 216_T_8 |
| 216_T_+1 | 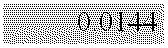 | 0.1974 | 0.0741 | 0.0772 | 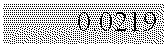 | 0.9021 | 216_T_+1 |
| 216_T_+2 | 0.3096 | 0.3468 | 0.1153 | 0.6344 | 0.1181 | 0.6945 | 216_T_+2 |
| 216_T_+4 | 0.2711 | 0.6529 | 0.2089 | 0.8494 | 0.3638 | 0.9405 | 216_T_+4 |
| 216_R_+2 | 0.1899 | 0.9553 | 0.519 | 0.6061 | 0.267 | 0.8387 | 216_R_+2 |
| 216_R_+1 | 0.3641 | 0.5165 | 0.2261 | 0.3177 | 0.061 | 0.4647 | 216_R_+1 |
| 216_R_2 | 0.1962 | 0.4256 | 0.2238 | 0.2998 | 0.0745 | 0.4318 | 216_R_2 |
| 216_R_1 | 0.2913 | 0.3177 | 0.2156 | 0.2523 | 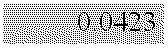 | 0.2934 | 216_R_1 |
| 216_QR_+7 | 0.1117 | 0.3534 | 0.2261 | 0.4609 | 0.1223 | 0.6626 | 216_QR_+7 |
| 216_QR_+6 | 0.0642 | 0.9403 | 0.1299 | 0.9983 | 0.2238 | 0.626 | 216_QR_+6 |
| 216_QR_+5 | 0.2914 | 216_{0.7561} | 0.1616 | 0.815 | 0.2255 | 0.8636 | —QR_+5 |
| 216_QR_+4 | 0.1253 | 0.2598 | 0.0624 | 0.3336 | 0.0825 | 0.2851 | 216_QR_+4 |
| 216_Q_+1 | 0.3082 | 0.4934 | 0.1278 | 0.8645 | 0.2558 | 0.8262 | 216_Q_+1 |
| 216_Q_2 | 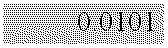 | 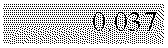 | 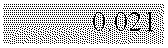 | 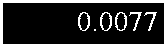 | 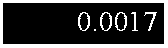 | 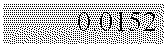 | 216_Q_2 |
| 216_Q_1 | 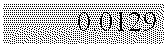 | 0.1534 | 0.0924 | 0.2463 | 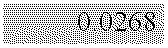 | 0.1185 | 216_Q_1 |
| 216_U_−1 | 0.0097 | 0.3354 | 0.1051 | 0.1615 | 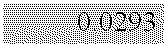 | 0.1401 | 216_U_−1 |
| 216_L_+1 | 0.2061 | 0.1806 | 0.1294 | 0.1884 | 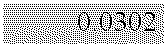 | 0.1887 | 216_L_+1 |
| 216_L_1 | 0.1542 | 0.9613 | 0.325 | 0.9999 | 0.3917 | 0.8772 | 216_L_1 |
| 216_L_−1 | 0.2675 | 0.3511 | 0.2591 | 0.2871 | 0.0528 | 0.1734 | 216_L_−1 |
| 216_L_−2 | 0.2354 | 0.8657 | 0.1417 | 0.8987 | 0.2811 | 0.8408 | 216_L_−2 |
| 216_V_+2 | 0.0558 | 0.3687 | 0.2253 | 0.4145 | 0.077 | 0.4394 | 216_V_+2 |
| 216_V_+1 | 0.1857 | 0.8927 | 0.2713 | 0.7385 | 0.2762 | 0.867 | 216_V_+1 |
| 216_I_1 | 0.0952 | 0.3777 | 0.1788 | 0.1961 | 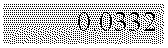 | 0.2355 | 216_I_1 |
| 216_G_−1 | — | 1 | 0.2373 | 0.9526 | 0.3934 | 0.982 | 216_G_−1 |
| 216_F_+1 | — | — | 0.1466 | 0.2595 | 0.0691 | 0.2676 | 216_F_+1 |
| 216_F_1 | — | — | — | 1 | 0.383 | 0.9241 | 216_F_1 |
| 216_D_1 | — | — | — | — | 0.2646 | 0.2646 | 216_D_1 |
| 216_D_−2 | — | — | — | — | — | 1 | 216_D_−2 |
| CNTL | 83.70% | 9.90% | 64.10% | 97.90% | 0.00% | 0.70% | CNTL |
| CASE | 91.00% | 10.20% | 73.30% | 97.90% | 1.00% | 1.00% | CASE |

TABLE 23

| | 216_T_2 | 216_T_3 | 216_T_4 | 216_T_5 | 216_T_6 | 216_T_7 | 216_T_8 | |
|---|---|---|---|---|---|---|---|---|
| 216_T_2 | 0.0514 | 0.1243 | 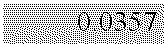 | 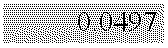 | 0.0787 | 0.0595 | 0.062 | 216_T_2 |
| 216_T_3 | — | 0.7369 | 0.4787 | 0.4804 | 0.6341 | 0.8292 | 0.8018 | 216_T_3 |
| 216_T_4 | — | — | 0.6065 | 0.3323 | 0.5394 | 0.9824 | 0.9942 | 216_T_4 |
| 216_T_5 | — | — | — | 0.6206 | 0.6544 | 0.168 | 0.2128 | 216_T_5 |
| 216_T_6 | — | — | — | — | 0.813 | 0.812 | 0.74 | 216_T_6 |
| 216_T_7 | — | — | — | — | — | 1 | 0.5041 | 216_T_7 |
| 216_T_8 | — | — | — | — | — | — | 1 | 216_T_8 |
| 216_T_+1 | — | — | — | — | — | — | — | 216_T_+1 |
| 216_T_+2 | — | — | — | — | — | — | — | 216_T_+2 |
| 216_T_+4 | — | — | — | — | — | — | — | 216_T_+4 |
| 216_R_+2 | — | — | — | — | — | — | — | 216_R_+2 |
| 216_R_+1 | — | — | — | — | — | — | — | 216_R_+1 |
| 216_R_2 | — | — | — | — | — | — | — | 216_R_2 |
| 216_R_1 | — | — | — | — | — | — | — | 216_R_1 |
| 216_QR_+7 | — | — | — | — | — | — | — | 216_QR_+7 |
| 216_QR_+6 | — | — | — | — | — | — | — | 216_QR_+6 |
| 216_QR_+5 | — | — | — | — | — | — | — | 216_QR_+5 |
| 216_QR_+4 | — | — | — | — | — | — | — | 216_QR_+4 |
| 216_Q_+1 | — | — | — | — | — | — | — | 216_Q_+1 |
| 216_Q_2 | — | — | — | — | — | — | — | 216_Q_2 |
| 216_Q_1 | — | — | — | — | — | — | — | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 67.80% | 9.50% | 94.70% | 79.20% | 76.60% | 94.20% | 93.90% | CNTL |
| CASE | 46.40% | 10.70% | 100.00% | 75.00% | 75.00% | 96.40% | 96.40% | CASE |
| | 216_T_+1 | 216_T_+2 | 216_T_+4 | 216_R_+2 | 216_R_+1 | 216_R_2 | 216_R_1 | |
| 216_T_2 | 0.1864 | 0.2019 | 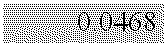 | 0.1509 | 0.1065 | 0.0837 | 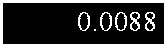 | 216_T_2 |
| 216_T_3 | 0.9402 | 0.9479 | 0.9244 | 0.1641 | 0.165 | 0.1963 | 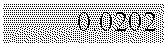 | 216_T_3 |
| 216_T_4 | 0.5099 | 0.3588 | 0.4219 | 0.1293 | 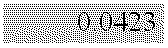 | 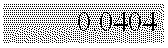 | 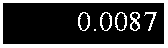 | 216_T_4 |
| 216_T_5 | 0.1058 | 0.5476 | 0.6693 | 0.3828 | 0.1102 | 0.1245 | 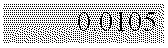 | 216_T_5 |
| 216_T_6 | 0.8376 | 0.7371 | 0.9089 | 0.5923 | 0.1377 | 0.1121 | 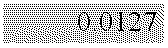 | 216_T_6 |
| 216_T_7 | 0.6321 | 0.7968 | 0.7783 | 0.3655 | 0.1388 | 0.1468 | 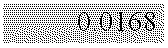 | 216_T_7 |
| 216_T_8 | 0.488 | 0.7896 | 0.7716 | 0.3796 | 0.1355 | 0.1386 | 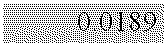 | 216_T_8 |
| 216_T_+1 | 0.5937 | 0.8346 | 0.6672 | 0.4505 | 0.1151 | 0.1286 | 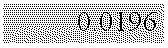 | 216_T_+1 |
| 216_T_+2 | — | 0.83 | 0.2834 | 0.4935 | 0.1588 | 0.1904 | 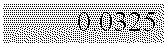 | 216_T_+2 |
| 216_T_+4 | — | — | 0.6296 | 0.4691 | 0.0794 | 0.1096 | 0.0096 | 216_T_+4 |
| 216_R_+2 | — | — | — | 0.4778 | 0.1289 | 0.14 | 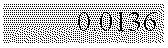 | 216_R_+2 |
| 216_R_+1 | — | — | — | — | 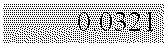 | 0.1017 | 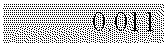 | 216_R_+1 |
| 216_R_2 | — | — | — | — | — | 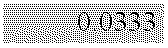 | 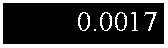 | 216_R_2 |
| 216_R_1 | — | — | — | — | — | — | 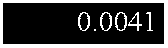 | 216_R_1 |
| 216_QR_+7 | — | — | — | — | — | — | — | 216_QR_+7 |
| 216_QR_+6 | — | — | — | — | — | — | — | 216_QR_+6 |
| 216_QR_+5 | — | — | — | — | — | — | — | 216_QR_+5 |
| 216_QR_+4 | — | — | — | — | — | — | — | 216_QR_+4 |
| 216_Q_+1 | — | — | — | — | — | — | — | 216_Q_+1 |
| 216_Q_2 | — | — | — | — | — | — | — | 216_Q_2 |
| 216_Q_1 | — | — | — | — | — | — | — | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 82.90% | 36.30% | 23.00% | 89.60% | 92.00% | 92.60% | 7.80% | CNTL |
| CASE | 78.60% | 32.10% | 28.60% | 96.40% | 76.90% | 78.60% | 28.60% | CASE |
| | 216_QR_+7 | 216_QR_+6 | 216_QR_+5 | 216_QR_+4 | 216_Q_+1 | 216_Q_2 | 216_Q_1 | |
| 216_T_2 | 0.0914 | 0.0506 | 0.1874 | 0.1924 | 0.1399 | 0.1647 | 0.2214 | 216_T_2 |
| 216_T_3 | 0.2541 | 0.9842 | 0.5592 | 0.3088 | 0.4792 | 0.1073 | 0.68 | 216_T_3 |
| 216_T_4 | 0.503 | 0.2862 | 0.2802 | 0.5248 | 0.2718 | 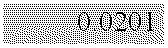 | 0.4625 | 216_T_4 |
| 216_T_5 | 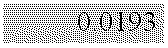 | 0.6959 | 0.5418 | 0.8568 | 0.4941 | 0.1575 | 0.6092 | 216_T_5 |
| 216_T_6 | 0.876 | 0.9981 | 0.6551 | 0.5037 | 0.5361 | 0.0844 | 0.9136 | 216_T_6 |
| 216_T_7 | 0.6745 | 0.7745 | 0.3781 | 0.7488 | 0.6372 | 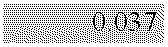 | 0.8639 | 216_T_7 |
| 216_T_8 | 0.5855 | 0.6202 | 0.3539 | 0.727 | 0.6365 | 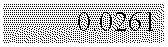 | 0.8702 | 216_T_8 |
| 216_T_+1 | 0.9187 | 0.6807 | 0.6064 | 0.9072 | 0.4903 | 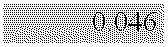 | 0.319 | 216_T_+1 |
| 216_T_+2 | 0.9216 | 0.7751 | 0.4055 | 0.6702 | 0.3989 | 0.0697 | 0.8958 | 216_T_+2 |
| 216_T_+4 | 0.8987 | 0.6591 | 0.6732 | 0.2344 | 0.5765 | 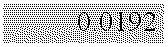 | 0.7132 | 216_T_+4 |
| 216_R_+2 | 0.2244 | 0.3101 | 0.146 | 0.4372 | 0.1157 | 0.0789 | 0.5406 | 216_R_+2 |
| 216_R_+1 | 0.0845 | 0.0921 | 0.1826 | 0.1505 | 0.1518 | 0.0838 | 0.1592 | 216_R_+1 |
| 216_R_2 | 0.1172 | 0.0961 | 0.1661 | 0.1518 | 0.1628 | 0.0866 | 0.1983 | 216_R_2 |
| 216_R_1 | 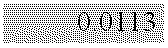 | 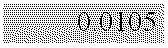 | 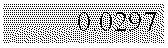 | 0.0255 | 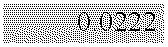 | 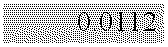 | 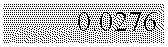 | 216_R_1 |
| 216_QR_+7 | 0.6391 | 0.7177 | 0.6406 | 0.8806 | 0.6188 | 0.0587 | 0.6982 | 216_QR_+7 |
| 216_QR_+6 | — | 1 | 0.4211 | 0.6283 | 0.3013 | 0.0784 | 0.8185 | 216_QR_+6 |
| 216_QR_+5 | — | — | 0.313 | 0.1105 | 0.2621 | 0.0591 | 0.4573 | 216_QR_+5 |
| 216_QR_+4 | — | — | — | 0.5371 | 0.1314 | 0.1379 | 0.7941 | 216_QR_+4 |
| 216_Q_+1 | — | — | — | — | 0.2724 | 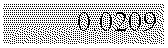 | 0.3082 | 216_Q_+1 |
| 216_Q_2 | — | — | — | — | — | 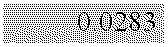 | 0.1055 | 216_Q_2 |
| 216_Q_1 | — | — | — | — | — | — | 0.725 | 216_Q_1 |
| 216_U_−1 | — | — | — | — | — | — | — | 216_U_−1 |
| 216_L_+1 | — | — | — | — | — | — | — | 216_L_+1 |
| 216_L_1 | — | — | — | — | — | — | — | 216_L_1 |
| 216_L_−1 | — | — | — | — | — | — | — | 216_L_−1 |
| 216_L_−2 | — | — | — | — | — | — | — | 216_L_−2 |
| 216_V_+2 | — | — | — | — | — | — | — | 216_V_+2 |
| 216_V_+1 | — | — | — | — | — | — | — | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 75.70% | 98.70% | 50.00% | 57.10% | 48.10% | 75.30% | 89.60% | CNTL |
| CASE | 71.40% | 100.00% | 39.30% | 64.30% | 62.50% | 53.60% | 87.50% | CASE |
| | 216_U_−1 | 216_L_+1 | 216_L_1 | 216_L_−1 | 216_L_−2 | 216_V_+2 | 216_V_+1 | |
| 216_T_2 | 0.1925 | 0.0531 | 0.0936 | 0.0568 | 0.0918 | 0.1241 | 0.1621 | 216_T_2 |
| 216_T_3 | 0.9027 | 0.1186 | 0.9966 | 0.1189 | 0.9483 | 0.5519 | 0.8271 | 216_T_3 |
| 216_T_4 | 0.4641 | 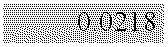 | 0.3384 | 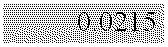 | 0.4347 | 0.2913 | 0.9791 | 216_T_4 |
| 216_T_5 | 0.1038 | 0.0531 | 0.8202 | 0.0576 | 0.1218 | 0.6548 | 0.1185 | 216_T_5 |
| 216_T_6 | 0.8376 | 0.0579 | 0.9799 | 0.0598 | 0.9969 | 0.3487 | 0.835 | 216_T_6 |
| 216_T_7 | 0.6497 | 0.0786 | 0.5075 | 0.0789 | 0.9077 | 0.6325 | 0.916 | 216_T_7 |
| 216_T_8 | 0.4836 | 0.0702 | 0.6191 | 0.075 | 0.8249 | 0.6225 | 0.8561 | 216_T_8 |
| 216_T_+1 | 0.5365 | 0.0598 | 0.7057 | 0.0622 | 0.7784 | 0.8165 | 0.7192 | 216_T_+1 |
| 216_T_+2 | 0.8408 | 0.099 | 0.7111 | 0.0971 | 0.7024 | 0.6255 | 0.8129 | 216_T_+2 |
| 216_T_+4 | 0.6565 | 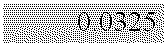 | 0.6815 | 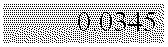 | 0.7815 | 0.8106 | 0.8199 | 216_T_+4 |
| 216_R_+2 | 0.4399 | 0.0636 | 0.3328 | 0.0661 | 0.111 | 0.4883 | 0.4012 | 216_R_+2 |
| 216_R_+1 | 0.1091 | 0.0633 | 0.1175 | 0.0602 | 0.179 | 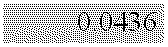 | 0.1427 | 216_R_+1 |
| 216_R_2 | 0.1344 | 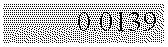 | 0.1472 | 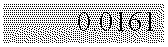 | 0.2042 | 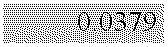 | 0.1459 | 216_R_2 |
| 216_R_1 | 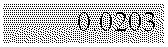 | 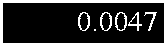 | 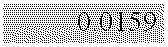 | 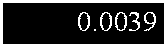 | 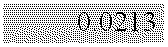 | 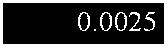 | 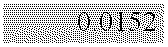 | 216_R_1 |
| 216_QR_+7 | 0.8618 | 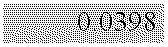 | 0.7941 | 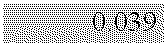 | 0.8881 | 0.7451 | 0.7462 | 216_QR_+7 |
| 216_QR_+6 | 0.6332 | 0.0691 | 0.4097 | 0.0702 | 0.9827 | 0.5193 | 0.7699 | 216_QR_+6 |
| 216_QR_+5 | 0.6112 | 0.1063 | 0.4489 | 0.106 | 0.569 | 0.4606 | 0.3673 | 216_QR_+5 |
| 216_QR_+4 | 0.9027 | 0.0812 | 0.5626 | 0.0816 | 0.8155 | 0.3467 | 0.7442 | 216_QR_+4 |
| 216_Q_+1 | 0.4922 | 0.0923 | 0.289 | 0.092 | 0.465 | 0.4652 | 0.6406 | 216_Q_+1 |
| 216_Q_2 | 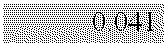 | 0.0587 | 0.0575 | 0.0558 | 0.0817 | 0.0825 | 0.0789 | 216_Q_2 |
| 216_Q_1 | 0.3265 | 0.1276 | 0.794 | 0.1285 | 0.5428 | 0.596 | 0.8505 | 216_Q_1 |
| 216_U_−1 | 0.591 | 0.0522 | 0.6716 | 0.0532 | 0.7612 | 0.8174 | 0.7175 | 216_U_−1 |
| 216_L_+1 | — | 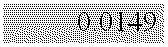 | 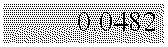 | 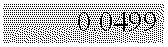 | 0.0875 | 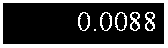 | 0.0745 | 216_L_+1 |
| 216_L_1 | — | — | 1 | 0.0517 | 0.9908 | 0.4566 | 0.4983 | 216_L_1 |
| 216_L_−1 | — | — | — | 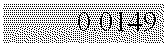 | 0.0877 | 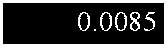 | 0.0776 | 216_L_−1 |
| 216_L_−2 | — | — | — | — | 1 | 0.5878 | 0.91 | 216_L_−2 |
| 216_V_+2 | — | — | — | — | — | 0.373 | 0.4953 | 216_V_+2 |
| 216_V_+1 | — | — | — | — | — | — | 1 | 216_V_+1 |
| 216_I_1 | — | — | — | — | — | — | — | 216_I_1 |
| 216_G_−1 | — | — | — | — | — | — | — | 216_G_−1 |
| 216_F_+1 | — | — | — | — | — | — | — | 216_F_+1 |
| 216_F_1 | — | — | — | — | — | — | — | 216_F_1 |
| 216_D_1 | — | — | — | — | — | — | — | 216_D_1 |
| 216_D_−2 | — | — | — | — | — | — | — | 216_D_−2 |
| CNTL | 83.10% | 92.00% | 0.60% | 92.00% | 6.70% | 70.80% | 94.20% | CNTL |
| CASE | 78.60% | 75.00% | 0.00% | 75.00% | 7.10% | 60.70% | 96.40% | CASE |

| | 216_I_1 | 216_G_−1 | 216_F_+1 | 216_F_1 | 216_D_1 | 216_D_−2 | |
|---|---|---|---|---|---|---|---|
| 216_T_2 | 0.0909 | 0.1637 | 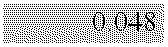 | 0.0815 | 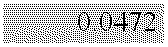 | 0.0683 | 216_T_2 |
| 216_T_3 | 0.1789 | 0.9677 | 0.1256 | 0.8419 | 0.9949 | 0.8539 | 216_T_3 |
| 216_T_4 | 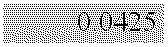 | 0.3741 | 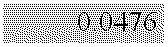 | 0.9752 | 0.2527 | 0.3207 | 216_T_4 |
| 216_T_5 | 0.0652 | 0.2079 | 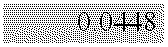 | 0.1373 | 0.7435 | 0.6039 | 216_T_5 |
| 216_T_6 | 0.0677 | 0.957 | 0.0943 | 0.8986 | 0.9784 | 0.7357 | 216_T_6 |
| 216_T_7 | 0.1029 | 0.7963 | 0.0923 | 0.9022 | 0.5217 | 0.6292 | 216_T_7 |
| 216_T_8 | 0.1003 | 0.819 | 0.0757 | 0.831 | 0.5288 | 0.578 | 216_T_8 |
| 216_T_+1 | 0.3281 | 0.7646 | 0.2019 | 0.6787 | 0.538 | 0.7074 | 216_T_+1 |
| 216_T_+2 | 0.1714 | 0.5849 | 0.2635 | 0.8532 | 0.7344 | 0.7752 | 216_T_+2 |
| 216_T_+4 | 0.067 | 0.5879 | 0.0594 | 0.8059 | 0.6085 | 0.591 | 216_T_+4 |
| 216_R_+2 | 0.0964 | 0.1258 | 0.1173 | 0.5041 | 0.2734 | 0.321 | 216_R_+2 |
| 216_R_+1 | 0.0515 | 0.1554 | 0.1161 | 0.1538 | 0.0672 | 0.1176 | 216_R_+1 |
| 216_R_2 | 0.1696 | 0.2001 | 0.0901 | 0.1632 | 0.0676 | 0.1272 | 216_R_2 |
| 216_R_1 | 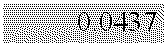 | 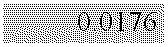 | 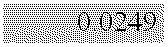 | 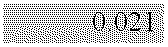 | 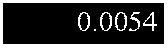 | 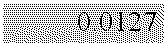 | 216_R_1 |
| 216_QR_+7 | 0.253 | 0.9131 | 0.1308 | 0.6922 | 0.7005 | 0.6339 | 216_QR_+7 |
| 216_QR_+6 | 0.0859 | 0.9127 | 0.0677 | 0.786 | 0.9687 | 0.6737 | 216_QR_+6 |
| 216_QR_+5 | 0.1808 | 0.6586 | 0.1822 | 0.4067 | 0.2538 | 0.3883 | 216_QR_+5 |
| 216_QR_+4 | 0.0755 | 0.7063 | 0.2482 | 0.7513 | 0.5206 | 0.61 | 216_QR_+4 |
| 216_Q_+1 | 0.1208 | 0.5445 | 0.1143 | 0.862 | 0.1418 | 0.2809 | 216_Q_+1 |
| 216_Q_2 | 0.0831 | 0.1175 | 0.1098 | 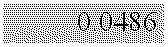 | 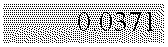 | 0.0583 | 216_Q_2 |
| 216_Q_1 | 0.1679 | 0.588 | 0.144 | 0.9111 | 0.7657 | 0.7939 | 216_Q_1 |
| 216_U_−1 | 0.3191 | 0.7498 | 0.2124 | 0.667 | 0.6025 | 0.5864 | 216_U_−1 |
| 216_L_+1 | 0.0845 | 0.0887 | 0.0692 | 0.0678 | 0.0509 | 0.0523 | 216_L_+1 |
| 216_L_1 | 0.1375 | 0.7513 | 0.1015 | 0.9959 | 0.9126 | 0.4148 | 216_L_1 |
| 216_L_−1 | 0.0846 | 0.085 | 0.0729 | 0.0718 | 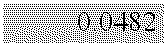 | 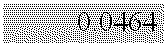 | 216_L_−1 |
| 216_L_−2 | 0.2211 | 0.6965 | 0.1255 | 0.9831 | 1 | 0.8513 | 216_L_−2 |
| 216_V_+2 | 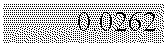 | 0.6322 | 0.118 | 0.5217 | 0.3071 | 0.3812 | 216_V_+2 |
| 216_V_+1 | 0.1025 | 0.7965 | 0.1808 | 0.9116 | 0.7034 | 0.6613 | 216_V_+1 |
| 216_I_1 | 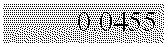 | 0.2247 | 0.1343 | 0.0604 | 0.0646 | 0.1262 | 216_I_1 |
| 216_G_−1 | — | 1 | 0.1806 | 0.9156 | 0.7415 | 0.739 | 216_G_−1 |
| 216_F_+1 | — | — | 0.051 | 0.107 | 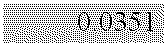 | 0.0799 | 216_F_+1 |
| 216_F_1 | — | — | — | 1 | 0.9127 | 0.8487 | 216_F_1 |
| 216_D_1 | — | — | — | — | 1 | 0.4883 | 216_D_1 |
| 216_D_−2 | — | — | — | — | — | 1 | 216_D_−2 |
| CNTL | 87.20% | 8.20% | 67.40% | 94.80% | 0.00% | 0.70% | CNTL |
| CASE | 71.40% | 7.10% | 46.40% | 96.40% | 0.00% | 0.00% | CASE |

### EXAMPLE 14

### Transmission Disequilibrium Test (TDT)

To ensure that the significant association observed in the case-control studies was not an artifact due to population admixture, a family based test of association, the transmission disequilibrium test (TDT) was conducted. By selecting a single affected offspring in each family, the TDT test performed a test of association (due to linkage disequilibrium) in the presence of linkage. The test determined whether a particular allele was preferentially transmitted to an affected individual over what would be expected by chance. Only heterozygous parents were considered informative for the TDT. In addition, to increase power, heterozygous parents transmitting a different allele to two affected offspring were ignored. Accordingly, the TDT would be based on the same families that contributed to the linkage signal. The significance levels were estimated by Markov Chain Monte Carlo simulation methods as implemented in TDTEX from the S.A.G.E. program (Department of Epidemiology and Biostatistics, Rammelkamp Center for Education and Research, MetroHealth Campus, Case Western Reserve University, Cleveland, Ohio (1997)).

1. Asthma Phenotype:

Five candidate SNPs were typed in the extended population in order to confirm the association seen in the case-control study. The five SNPs were in Gene 216 exons T5, T8, T+1, R1, and Q1. Since only heterozygote parents contribute information to the TDT test, SNP haplotypes (all 2-at-a-time and all 3-at-a-time) were constructed based on family data with the program GENEHUNTER (Kruglyak et al., 1996) in addition to analyzing the SNPs separately. This served to increase the informativeness of the single SNPs. These haplotypes were then used as “alleles” in future TDT analyses. In addition, p-values obtained from the TDT analyses were compared to the p-values obtained from the haplotyping in the case/control setting. To check for consistency, the p-values were recorded to compare the haplotype frequencies between the cases and controls of the over-transmitted alleles/haplotypes.

The TDT results strongly supported the association previously observed in the case control studies (Table 24). Three of the five SNPs showed alleles that were preferentially transmitted to affected offspring (p<0.04 to <0.0044) in either the combined or UK population. When these SNPs were haplotyped together, most combinations had a haplotype that was preferentially transmitted to affected offspring (p<0.03 to <0.001). The most significant haplotype in the combined population was composed of SNPs T+1/R1/Q1 (p=0.0006). The most significant haplotype in the UK population was composed of SNPs T5/R1/Q1 (p=0.0005). In contrast to the UK population, none of the single SNP allele or multiple SNP haplotypes were preferentially over-transmitted to affected offspring at significant levels in the US population. This is most likely due to the combination of reduced power of the TDT versus the case-control study and the smaller sample size in the US.

Importantly, for all of the single SNP or multiple SNP haplotypes the allele that was significantly over-transmitted in either the combined population or in the UK sample was more frequent in the cases than in the controls. A summary of the TDT analyses and a comparison between the Case/control and TDT results are presented in Table 24.

2. Bronchial Hyper-Responsiveness:

The TDT analyses were repeated using only those asthmatic pairs that satisfied the additional criteria of having a PC₂₀≦16 mg/ml (Table 25). The vast majority of single SNP and multiple SNP haplotypes showed increased significance with the more restricted phenotype. P values reached levels of <0.00008 for T5 μl/Q1 in the combined population and p<0.000008 in the UK sample. Similar to the yes/no phenotype, for the majority of the alleles in both the combined and UK population, the over-transmitted alleles in the TDT were more frequent in the cases. Similar to the yes/no phenotype with the less powerful TDT test, no significant results were observed with smaller US sample. In summary, the analysis of single SNPs and SNP haplotypes by the TDT test provided confirmatory evidence for Gene 216 as an asthma susceptibility gene.

TABLE 24
Asthma Yes/NO
Over-Transmitted
Haplotype

| | | | | |
|---|---|---|---|---|
| Exon in | TDT | Case/Control | Control | |
| Gene 216 | p-value | p-value | Frequency | Case Frequency |

Combined
US and UK

| | | | | |
|---|---|---|---|---|
| Q_1 | 0.0337 | 0.0213 | 89.5% | 94.8% |
| R_1 | 0.0725 | NS | 88.7% | 88.2% |
| T_+ 1 | 0.0956 | 0.0055 | 85.2% | 92.4% |
| T_8 | 1.0000 | NS | NA | |
| T_5 | 0.1364 | 0.0420 | 76.7% | 83.3% |
| R1Q1 | 0.0042 | 0.1362 | 78.2% | 83.1% |
| T + 1Q1 | 0.0932 | 0.0049 | 85.2% | 92.4% |
| T8Q1 | 0.0553 | 0.0084 | 86.0% | 92.9% |
| T5Q1 | 0.2659 | 0.0342 | 76.2% | 83.0% |
| T + 1R1 | 0.0029 | 0.0465 | 73.9% | 80.6% |
| T8R1 | 0.0799 | NS | 85.1% | 67.9% |
| T5R1 | 0.0107 | 0.1537 | 66.1% | 71.5% |
| T8T + 1 | 0.2762 | 0.0044 | 85.2% | 92.4% |
| T5T + 1 | 0.3078 | 0.0012 | 72.5% | 83.0% |
| T5T8 | 0.0948 | 0.0028 | 73.7% | 83.4% |
| T + 1R1Q1 | 0.0006 | 0.0430 | 73.9% | 80.8% |
| T8R1Q1 | 0.0086 | 0.0552 | 74.7% | 81.2% |
| T5R1Q1 | 0.0025 | 0.1591 | 65.9% | 71.2% |
| T5T + 1R1 | 0.0136 | 0.0175 | 62.3% | 71.2% |
| T8T + 1R1 | 0.0084 | 0.0377 | 73.9% | 80.9% |
| T5T8R1 | 0.0060 | 0.0235 | 63.0% | 71.5% |
| T5T8Q1 | 0.1242 | 0.0033 | 73.1% | 83.0% |
| T5T8T + 1 | 0.1540 | 0.0009 | 72.7% | 83.0% |
| T8T + 1Q1 | 0.1351 | 0.0043 | 85.3% | 92.4% |
| T5T + 1Q1 | 0.1080 | 0.0010 | 72.5% | 83.0% |

UK

| | | | | |
|---|---|---|---|---|
| Q_1 | 0.0044 | 0.0274 | 89.4% | 95.1% |
| R_1 | 0.3665 | 0.1473 | 86.8% | 91.4% |
| T_+ 1 | 0.0128 | 0.0105 | 86.4% | 93.8% |
| T_8 | 1.0000 | NS | NA | |
| T_5 | 0.0434 | 0.0426 | 75.4% | 83.3% |
| R1Q1 | 0.0044 | 0.0069 | 76.2% | 86.5% |
| T + 1Q1 | 0.0714 | 0.0066 | 86.4% | 93.8% |
| T8Q1 | 0.0342 | 0.0275 | 87.4% | 93.6% |
| T5Q1 | 0.1687 | 0.0314 | 74.9% | 82.9% |
| T + 1R1 | 0.0269 | 0.0018 | 73.2% | 85.1% |
| T8R1 | 0.4848 | 0.0933 | 84.6% | 89.9% |
| T5R1 | 0.0639 | 0.0067 | 63.1% | 74.7% |
| T8T + 1 | 0.2254 | 0.0069 | 86.4% | 93.8% |
| T5T + 1 | 0.2007 | 0.0088 | 72.9% | 82.9% |
| T5T8 | 0.0277 | 0.0103 | 73.7% | 83.4% |
| T + 1R1Q1 | 0.0063 | 0.0016 | 73.2% | 85.1% |
| T8R1Q1 | 0.0139 | 0.0039 | 74.1% | 85.0% |
| T5R1Q1 | 0.0005 | 0.0136 | 63.4% | 74.2% |
| T5T + 1R1 | 0.0220 | 0.0036 | 61.5% | 74.2% |
| T8T + 1R1 | 0.0043 | 0.0012 | 73.2% | 85.1% |
| T5T8R1 | 0.0095 | 0.0018 | 61.5% | 74.7% |
| T5T8Q1 | 0.0074 | 0.0105 | 73.3% | 82.9% |
| T5T8T + 1 | 0.0255 | 0.0082 | 73.0% | 82.9% |
| T8T + 1Q1 | 0.0207 | 0.0087 | 86.4% | 93.8% |
| T5T + 1Q1 | 0.0127 | 0.0093 | 72.9% | 82.9% |

US

| | | | | |
|---|---|---|---|---|
| Q_1 | 0.8039 | NS | 10.4% | 6.3% |
| R_1 | 0.1067 | NS | 92.2% | 75.9% |
| T_+ 1 | 0.6288 | NS | 17.1% | 13.0% |
| T_8 | 1.0000 | NS | NA | |
| T_5 | 0.7020 | NS | 20.8% | 16.7% |
| R1Q1 | 0.2134 | NS | 81.8% | 69.6% |
| T + 1Q1 | 0.6811 | NS | 10.4% | 9.7% |
| T8Q1 | 0.7584 | 0.2887 | 83.6% | 90.2% |
| T5Q1 | 0.8284 | NS | 9.7% | 8.3% |
| T + 1R1 | 0.0658 | NS | 75.1% | 63.0% |
| T8R1 | 0.0687 | NS | 86.1% | 72.2% |
| T5R1 | 0.1859 | NS | 71.4% | 59.3% |
| T8T + 1 | 0.9465 | 0.4778 | 83.0% | 87.0% |
| T5T + 1 | 0.8537 | 0.5074 | 9.7% | 13.0% |
| T5T8 | 0.8848 | NS | 20.8% | 13.0% |
| T + 1R1Q1 | 0.1569 | NS | 75.2% | 62.7% |
| T8R1Q1 | 0.2386 | NS | 75.8% | 66.0% |
| T5R1Q1 | 0.0831 | NS | 70.7% | 59.3% |
| T5T + 1R1 | 0.1332 | NS | 64.1% | 59.9% |
| T8T + 1R1 | 0.1299 | NS | 75.2% | 63.4% |
| T5T8R1 | 0.0813 | NS | 65.5% | 60.2% |
| T5T8Q1 | 0.8654 | NS | 9.7% | 7.8% |
| T5T8T + 1 | 0.8546 | NS | 9.6% | 9.3% |
| T8T + 1Q1 | 0.6864 | NS | 10.4% | 9.3% |
| T5T + 1Q1 | 0.8618 | 0.9991 | 9.7% | 9.7% |

NS = non-significant or over-transmitted allele not present more often in cases than controls;
NA = no alleles were over-transmitted

TABLE 25
BHR
Over-Transmitted
Haplotype

| | | | | |
|---|---|---|---|---|
| Exon in | TDT | Case/Control | Control | |
| Gene 216 | p-value | p-value | Frequency | Case Frequency |

Combined US
and UK

| | | | | |
|---|---|---|---|---|
| Q_1 | 0.0800 | 0.1565 | 89.5% | 94.2% |
| R_1 | 0.0374 | NS | 88.7% | 88.3% |
| T_+ 1 | 0.1252 | 0.1413 | 85.2% | 90.6% |
| T_8 | 1.0000 | NS | NA | |
| T_5 | 0.0947 | 0.4681 | 76.7% | 80.2% |
| R1Q1 | 0.0017 | 0.2040 | 78.2% | 83.7% |
| T + 1Q1 | 0.1835 | 0.1192 | 85.2% | 90.6% |
| T8Q1 | 0.1616 | 0.0987 | 86.0% | 91.8% |
| T5Q1 | 0.1496 | 0.3214 | 76.2% | 80.2% |
| T + 1R1 | 0.0015 | 0.1479 | 73.9% | 80.2% |
| T8R1 | 0.0281 | 0.7994 | 85.1% | 85.9% |
| T5R1 | 0.0009 | 0.6419 | 66.1% | 68.4% |
| T8T + 1 | 0.6224 | 0.1380 | 85.2% | 90.6% |
| T5T + 1 | 0.4821 | 0.0660 | 72.5% | 80.3% |
| T5T8 | 0.1786 | 0.1284 | 73.7% | 80.2% |
| T + 1R1Q1 | 0.0003 | 0.1426 | 73.9% | 80.4% |
| T8R1Q1 | 0.0035 | 0.1298 | 74.7% | 81.4% |
| T5R1Q1 | 0.0001 | 0.4524 | 65.9% | 69.7% |
| T5T + 1R1 | 0.0052 | 0.1332 | 62.3% | 69.6% |
| T8T + 1R1 | 0.0066 | 0.1397 | 73.9% | 80.6% |
| T5T8R1 | 0.0028 | 0.2632 | 63.0% | 68.4% |
| T5T8Q1 | 0.3680 | 0.0954 | 73.1% | 80.3% |
| T5T8T + 1 | 0.5282 | 0.0786 | 72.7% | 80.3% |
| T8T + 1Q1 | 0.3105 | 0.1261 | 85.3% | 90.6% |
| T5T + 1Q1 | 0.5276 | 0.0686 | 72.5% | 80.3% |

UK

| | | | | |
|---|---|---|---|---|
| Q_1 | 0.0069 | 0.0613 | 89.4% | 95.8% |
| R_1 | 0.3285 | 0.1041 | 86.8% | 93.0% |
| T_+ 1 | 0.0201 | 0.0454 | 86.4% | 94.0% |
| T_8 | 1.0000 | NS | NA | |
| T_5 | 0.0367 | 0.2644 | 75.4% | 81.6% |
| R1Q1 | 0.00078 | 0.0052 | 76.2% | 89.8% |
| T + 1Q1 | 0.0209 | 0.0280 | 86.4% | 94.0% |
| T8Q1 | 0.0120 | 0.0933 | 87.4% | 93.8% |
| T5Q1 | 0.0974 | 0.1624 | 74.9% | 81.7% |
| T + 1R1 | 0.0001 | 0.0026 | 73.2% | 87.6% |
| T8R1 | 0.2818 | 0.1182 | 84.6% | 91.0% |
| T5R1 | 0.0038 | 0.0420 | 63.1% | 74.6% |
| T8T + 1 | 0.1437 | 0.0327 | 86.4% | 94.0% |
| T5T + 1 | 0.0902 | 0.0739 | 72.9% | 81.7% |
| T5T8 | 0.0536 | 0.1052 | 73.7% | 81.7% |
| T + 1R1Q1 | 0.000075 | 0.0042 | 73.2% | 87.8% |
| T8R1Q1 | 0.0031 | 0.0056 | 74.1% | 87.7% |
| T5R1Q1 | 0.0000078 | 0.0331 | 63.4% | 75.4% |
| T5T + 1R1 | 0.0071 | 0.0131 | 61.5% | 75.3% |
| T8T + 1R1 | 0.0023 | 0.0034 | 73.2% | 87.8% |
| T5T8R1 | 0.0073 | 0.0216 | 61.5% | 74.6% |
| T5T8Q1 | 0.0424 | 0.0835 | 73.3% | 81.7% |
| T5T8T + 1 | 0.1380 | 0.0761 | 73.0% | 81.7% |
| T8T + 1Q1 | 0.0322 | 0.0319 | 86.4% | 94.0% |
| T5T + 1Q1 | 0.1096 | 0.0756 | 72.9% | 81.7% |

US

| | | | | |
|---|---|---|---|---|
| Q_1 | 0.5081 | 0.7250 | 10.4% | 12.5% |
| R_1 | 0.0577 | NS | 92.2% | 71.4% |
| T_+ 1 | 0.5493 | 0.5937 | 17.1% | 21.4% |
| T_8 | 1.0000 | NS | NA | |
| T_5 | 0.7741 | 0.6206 | 20.8% | 25.0% |
| R1Q1 | 0.1259 | NS | 81.8% | 58.8% |
| T + 1Q1 | 0.7495 | 0.1224 | 10.4% | 21.4% |
| T8Q1 | 0.7514 | 0.7864 | 10.4% | 12.1% |
| T5Q1 | 0.1029 | 0.1408 | 9.7% | 18.8% |
| T + 1R1 | 0.2012 | NS | 75.1% | 50.0% |
| T8R1 | 0.0880 | NS | 86.1% | 67.9% |
| T5R1 | 0.0963 | NS | 71.4% | 46.4% |
| T8T + 1 | 0.7557 | 0.2626 | 10.7% | 17.9% |
| T5T + 1 | 0.4904 | 0.0908 | 9.7% | 21.4% |
| T5T8 | 0.8871 | 0.9876 | 20.8% | 21.4% |
| T + 1R1Q1 | 0.0828 | NS | 75.2% | 50.0% |
| T8R1Q1 | 0.1759 | NS | 75.8% | 55.9% |
| T5R1Q1 | 0.2046 | NS | 70.7% | 46.4% |
| T5T + 1R1 | 0.1915 | NS | 64.1% | 46.4% |
| T8T + 1R1 | 0.2537 | NS | 75.2% | 50.0% |
| T5T8R1 | 0.1633 | NS | 65.5% | 46.4% |
| T5T8Q1 | 0.6920 | 0.3863 | 9.7% | 16.1% |
| T5T8T + 1 | 0.8586 | 0.3158 | 9.6% | 17.9% |
| T8T + 1Q1 | 0.7517 | 0.3367 | 10.4% | 17.9% |
| T5T + 1Q1 | 0.8579 | 0.1166 | 9.7% | 21.4% |

NS = non-significant or over-transmitted allele not present more often in cases than controls;
NA = no alleles were over-transmitted

### EXAMPLE 15

### Attributable Risk Assessment

From the knowledge of the frequency of a functional polymorphism and the relative risk of the heterozygote and homozygote (at-risk) genotypes, one can evaluate the attributable fraction (M. J. Khoury et al., 1993, Fundamentals of Genetic Epidemiology, J. L. Kelsy et al., (eds), Monographs in Epidemiology and Biostatistics, Oxford University Press, New York, N.Y., Section 3, pp 74-77) or attributable risk in the population. An attributable fraction of 25% would mean that if the population were monomorphic for the protective allele, the prevalence of the trait would be 25% lower.

The formula for the attributable fraction is:

$$Attributable fraction = \frac{{( 1 - f )}^{2} + 2 f ( 1 - f ) γ + {f}^{2} η - 1}{{( 1 - f )}^{2} + 2 f ( 1 - f ) γ + {f}^{2} η} ,$$

where f is the allele frequency, y is the relative risk of the heterozygote genotype over the wild type homozygote, and η is the risk of the homozygote mutant over the wild type homozygote. This approach requires the estimation of f, γ and η. Ideally these quantities should be estimated in an epidemiological sample.

The study design (genome scan with affected sibling pairs followed by association study using IBD=2 individuals as cases in the case/control comparison) offers maximum power to detect linkage and association, but does not provide estimates of the required parameters, namely 1) the relative risk (or odds ratio) of the genotype/allele for most SNPs or haplotypes and 2) the frequency of the SNP in the general population. In a recent paper, Altshuler et al. used the data from a TDT analysis to estimate allele and genotype relative risks assuming a multiplicative model or ρ=γ² (D. Altshuler et al., 2000, Nature Genetics 26:76-80). Thus, the mutant homozygote is predicted to carry a relative risk equal to the square of the risk for the heterozygote.

To overcome some of the difficulties mentioned above that are associated with a case/control design, the data obtained from typing 5 SNPs in Gene 216 on the entire population (not just the subset of IBD=2 individuals) were used to estimate the relative risk of these 5 SNPs. The data from the TDT obtained by using the first asthmatic sibling per family were used. Because of the limited number of informative matings in the TDT analysis, a multiplicative model for the genotype relative risk was used as in the Altshuler et. al paper, i.e. η=γ². An interval on the attributable fraction estimates was made by constructing individual confidence regions for the allele frequency in the control population and for the attributable risk obtained from the TDT data. While combining these two confidence intervals to obtain a confidence region for the attributable fraction did not lead to a proper confidence region with the required coverage, it determined the variability involved in estimating the attributable fraction. As a short hand notation, this is referred to as a confidence interval with coverage equal to the one used for the constituent parameters.

By using the control population to estimate allele frequencies, the attributable risk was underestimated. Based on these assumptions, the attributable risk for the single SNPs that were significant in the case-control study (p<0.05) in either population was computed. The AF was also computed for all SNP combinations significant in the combined TDT analysis (p<0.01) using the asthma phenotype. These values are shown below.

| | Attributable | |
|---|---|---|
| SNP(s) | fraction (AF) estimate | 80% Confidence Interval |
| Q1 | 50% | 17 to 65% |
| R1 | 37% | 4 to 57% |
| T + 1 | 39% | 7 to 57% |
| T5 | 22% | 0 to 35% |
| R1Q1 | 36% | 14 to 54% |
| T + 1R1 | 29% | 8 to 47% |
| T + 1R1Q1 | 34% | 14 to 52% |
| T5R1Q1 | 19% | 3 to 38% |
| T5T8R1 | 24% | 9 to 41% |
| T8R1Q1 | 32% | 11 to 50% |
| T8T + 1R1 | 25% | 2 to 44% |

Because the alleles that confer increased risk of developing asthma are so common (haplotype frequencies ranging from 60% to 83%), their effect translated into a substantial population attributable risk, with estimates ranging from 19 to 50% for different SNPs or SNP haplotypes. These computations depended heavily on allele frequency and risk estimates. Proper estimates of the attributable fraction are based on a population sample and are only meaningful for functional SNPs or SNP haplotypes.

Conclusion:

Gene 216 has been demonstrated to be an asthma gene in accordance with the data disclosed herein, including: 1) localization to a region on chromosome 20 identified through linkage; 2) polymorphism analysis performed to identify sequence variants localized in the candidate gene; 3) genotype analyses of the identified polymorphisms; 4) association between identified alleles and the asthma phenotype in a case-control analysis; 5) association between identified alleles and the asthma phenotype in transmission disequilibrium tests (TDT), haplotype analyses, and analyses using additional phenotypes; 6) identification of transcripts in tissues relevant to pulmonary disease and/or inflammation; and 7) characterization of Gene 216 as an ADAM family member. In addition to respiratory diseases, Gene 216 is likely to be involved in obesity and inflammatory bowel disease, as obesity (Wilson et al., 1999, Arch. Intern. Med. 159: 2513-14) and inflammatory bowel disease (B. Wallaert et al., 1995, J. Exp. Med. 182:1897-1904) have been linked to asthma.

### EXAMPLE 16

### Protein Expression and Purification

Expression and purification of the Gene 216 protein of the invention can be performed essentially as outlined below. To facilitate the cloning, expression, and purification of membrane and secreted protein from the 20p13-p12, a gene expression system, such as the pET System (Novagen), for cloning and expression of recombinant proteins in E. coli is selected. Also, a DNA sequence encoding a peptide tag, the His-Tap, is fused to the 3′ end of DNA sequences of interest to facilitate purification of the recombinant protein products. The 3′ end is selected for fusion to avoid alteration of any 5′ terminal signal sequence.

Nucleic acids chosen, for example, from the nucleic acids set forth in SEQ ID NO:1 or SEQ ID NO:6 (FIGS. 24 and 29, respectively) for cloning the genes are prepared by polymerase chain reaction (PCR). Synthetic oligonucleotide primers specific for the 5′ and 3′ ends of the nucleotide sequences are designed and purchased from Life Technologies. All forward primers (specific for the 5′ end of the sequence) are designed to include an NcoI cloning site at the 5′ terminus. These primers are designed to permit initiation of protein translation at the methionine residue encoded within the NcoI site followed by a valine residue and the protein encoded by the DNA sequence. All reverse primers (specific for the 3′ end of the sequence) include an EcoRI site at the 5′ terminus to permit cloning of the sequence into the reading frame of the pET-28b. The pET-28b vector provides a sequence encoding an additional 20 carboxyl-terminal amino acids including six histidine residues (at the C-terminus), which comprise the histidine affinity tag.

DNA prepared from the 20p13-p12 region is used as the source of template DNA for PCR amplification (Ausubel et al., 1994). To amplify a DNA sequence containing the nucleotide sequence, c DNA (50 ng) is introduced into a reaction vial containing 2 mM MgCl₂, 1 μM synthetic oligonucleotide primers (forward and reverse primers) complementary to and flanking a defined 20p13-p12 region, 0.2 mM of each of deoxynucleotide triphosphate, dATP, dGTP, dCTP, dTTP and 2.5 units of heat stable DNA polymerase (Amplitaq, Roche Molecular Systems, Inc., Branchburg, N.J.) in a final volume of 100 microliters.

Upon completion of thermal cycling reactions, each sample of amplified DNA is purified using the Qiaquick Spin PCR purification kit. All amplified DNA samples are subjected to digestion with the restriction endonucleases, e.g., NcoI and EcoRI (NEB) (Ausubel et al., 1994). DNA samples are then subjected to electrophoresis on 1.0% NuSeive (FMC BioProducts) agarose gels. DNA is visualized by exposure to ethidium bromide and long wave UV irradiation. DNA contained in slices isolated from the agarose gel was purified using the BIO 101 GeneClean Kit protocol.

The pET-28b vector is prepared for cloning by digestion with restriction endonucleases, e.g., NcoI and EcoRI (NEB) (Ausubel et al., 1994). The pET-28a vector, which encodes the histidine affinity tag that can be fused to the 5′ end of an inserted gene, is prepared by digestion with appropriate restriction endonucleases.

Following digestion, DNA inserts are cloned (Ausubel et al., 1994) into the previously digested pET-28b expression vector. Products of the ligation reaction are then used to transform the BL21 strain of E. coli (Ausubel et al., 1994) as described below.

Competent bacteria, E. coli strain BL21 or E. coli strain BL21 (DE3), are transformed with recombinant pET expression plasmids carrying the cloned sequence according to standard methods (Ausubel et al., 1994). Briefly, 1 microliter of ligation reaction is mixed with 50 microliters of electrocompetent cells and subjected to a high voltage pulse, after which samples were incubated in 0.45 ml SOC medium (0.5% yeast extract, 2.0% tryptone, 10 mM NaCl, 2.5 mM KCl, 10 mM MgCl₂, 10 mM MgSO₄ and 20 mM glucose) at 37° C. with shaking for 1 hr. Samples are then spread on LB agar plates containing 25 μg/ml kanamycin sulfate for growth overnight. Transformed colonies of BL21 are then picked and analyzed to evaluate cloned inserts, as described below.

Individual BL21 clones transformed with recombinant pET-28b. 20p13-p12 region nucleotide sequences are analyzed by PCR amplification of the cloned inserts using the same forward and reverse primers specific for the 20p13-p12 region sequences that are used in the original PCR amplification cloning reactions. Successful amplification verifies the integration of the sequence in the expression vector (Ausubel et al., 1994).

Individual clones of recombinant pET-28b vectors carrying properly cloned 20p13-p12 region nucleotide sequences are picked and incubated in 5 ml of LB broth plus 25 μg/ml kanamycin sulfate overnight. The following day plasmid DNA is isolated and purified using the QIAGEN plasmid purification protocol.

The pET vector can be propagated in any E. coli K-12 strain, e.g., HMS174, HB101, JM109, DH5, and the like, for purposes of cloning or plasmid preparation. Hosts for expression include E. coli strains containing a chromosomal copy of the gene for T7 RNA polymerase. These hosts are lysogens of bacteriophage DE3, a lambda derivative that carries the lad gene, the lacUV5 promoter, and the gene for T7 RNA polymerase. T7 RNA polymerase is induced by addition of isopropyl-β-D-thiogalactoside (IPTG), and the T7 RNA polymerase transcribes any target plasmid containing a functional T7 promoter, such as pET-28b, carrying its gene of interest. Strains include, for example, BL21(DE3) (Studier et al., 1990, Meth. Enzymol., 185:60-89).

To express the recombinant sequence, 50 ng of plasmid DNA are isolated as described above to transform competent BL21(DE3) bacteria as described above (provided by Novagen as part of the pET expression kit). The lacZ gene (β-galactosidase) is expressed in the pET-System as described for the 20p13-p12 region recombinant constructions. Transformed cells were cultured in SOC medium for 1 hr, and the culture is then plated on LB plates containing 25 μg/ml kanamycin sulfate. The following day, the bacterial colonies are pooled and grown in LB medium containing kanamycin sulfate (25 μg/ml) to an optical density at 600 nM of 0.5 to 1.0 OD units, at which point 1 mM IPTG was added to the culture for 3 hr to induce gene expression of the 20p13-p12 region recombinant DNA constructions.

After induction of gene expression with IPTG, bacteria are collected by centrifugation in a Sorvall RC-3B centrifuge at 3500× g for 15 min at 4° C. Pellets are resuspended in 50 ml of cold mM Tris-HCl, pH 8.0, 0.1 M NaCl and 0.1 mM EDTA (STE buffer). Cells are then centrifuged at 2000× g for 20 min at 4° C. Wet pellets are weighed and frozen at −8° C. until ready for protein purification.

A variety of methodologies known in the art can be used to purify the isolated proteins (Coligan et al., 1995, Current Protocols in Protein Science, John Wiley & Sons, New York, N.Y.). For example, the frozen cells can be thawed, resuspended in buffer, and ruptured by several passages through a small volume microfluidizer (Model M-110S, Microfluidics International Corp., Newton, MA). The resultant homogenate is centrifuged to yield a clear supernatant (crude extract) and, following filtration, the crude extract is fractioned over columns. Fractions are monitored by absorbance at OD₂₈₀ nm and peak fractions may be analyzed by SDS-PAGE.

The concentrations of purified protein preparations are quantified spectrophotometrically using absorbance coefficients calculated from amino acid content (Perkins, 1986, Eur. J. Biochem., 157:169-180). Protein concentrations are also measured by the method of Bradford, 1976, Anal. Biochem., 72:248-254; and Lowry et al., 1951, J. Biol. Chem., 193:265-275 using bovine serum albumin as a standard.

SDS-polyacrylamide gels of various concentrations are purchased from Bio-Rad, and stained with Coomassie blue. Molecular weight markers may include rabbit skeletal muscle myosin (200 kDa), E. coli β-galactosidase (116 kDa), rabbit muscle phosphorylase B (97.4 kDa), bovine serum albumin (66.2 kDa), ovalbumin (45 kDa), bovine carbonic anyhdrase (31 kDa), soybean trypsin inhibitor (21.5 kDa), egg white lysozyme (14.4 kDa) and bovine aprotinin (6.5 kDa).

Proteins can also be isolated by other conventional means of protein biochemistry and purification to obtain a substantially pure product, i.e., 80, 95, or 99% free of cell component contaminants, as described in Jacoby, 1984, Methods in Enzymology, Vol. 104, Academic Press, NY; Scoopes, 1987, Protein Purification, Principles and Practice, 2^{nd} Ed., Springer-Verlag, NY; and Deutscher (ed), 1990, Guide to Protein Purification, Methods in Enzymology, Vol. 182. If the protein is secreted, it can be isolated from the supernatant in which the host cell is grown; otherwise, it can be isolated from a lysate of the host cells.

Once a sufficient quantity of the desired protein has been obtained, it may be used for various purposes. One use of the protein or polypeptide is the production of antibodies specific for binding. These antibodies may be either polyclonal or monoclonal, and may be produced by in vitro or in vivo techniques well known in the art. Monoclonal antibodies to epitopes of any of the peptides identified and isolated as described can be prepared from murine hybridomas (Kohler, 1975, Nature, 256:495). In summary, a mouse is inoculated with a few micrograms of protein over a period of 2 weeks. The mouse is then sacrificed. The cells that produce antibodies are then removed from the mouse's spleen. The spleen cells are then fused with polyethylene glycol with mouse myeloma cells. The successfully fused cells are diluted in a microtiter plate and growth of the culture is continued. The amount of antibody per well is measured by immunoassay methods such as ELISA (Engvall, 1980, Meth. Enzymol., 70:419). Clones producing antibody can be expanded and further propagated to produce protein antibodies. Other suitable techniques involve in vitro exposure of lymphocytes to the antigenic polypeptides, or alternatively, to selection of libraries of antibodies in phage or similar vectors. See Huse et al., 1989, Science, 246:1275-1281. For additional information on antibody production see Davis et al., 1989, Basic Methods in Molecular Biology, Elsevier, N.Y., Section 21-2. Such antibodies are particularly useful in diagnostic assays for detection of variant protein forms, or as an active ingredient in a pharmaceutical composition.

The disclosure of each of the patents, patent applications, and publications cited in the specification is hereby incorporated by reference herein in its entirety.

Although the invention has been set forth in detail, one skilled in the art will recognize that numerous changes and modifications can be made, and that such changes and modifications may be made without departing from the spirit and scope of the invention.

What is claimed is:

1. An isolated nucleic acid comprising a nucleotide sequence selected from the group consisting of:
a. SEQ ID NO:1;
b. a nucleotide sequence encoding amino acid SEQ ID NO:4;
c. a nucleotide sequence complementary to SEQ ID NO:1;
d. a nucleotide sequence which hybridizes under high stringency conditions to SEQ ID NO:1;
e. a nucleotide sequence which hybridizes under moderate stringency conditions to SEQ ID NO:1;
f. a nucleotide sequence which hybridizes under low stringency conditions to SEQ ID NO:1;
g. a nucleotide sequence which is at least 95% identical to the sequence of SEQ ID NO:1;
h. a nucleotide sequence which is at least 80% identical to the sequence of SEQ ID NO:1; and
i. a nucleotide sequence which is at least 50% identical to the sequence of SEQ ID NO:1.

2. The isolated nucleic acid of claim 1 which is DNA.

3. The isolated nucleic acid of claim 1 which is RNA.

4. A vector comprising the isolated nucleic acid of claim 1.

5. A host cell comprising the expression vector of claim 4.

6. The host cell of claim 5 which is selected from the group consisting of eukaryotic and prokaryotic cells.

7. The host cell of claim 5 which is selected from the group consisting of bacterial, fungal.

8. The isolated nucleic acid of claim 1, wherein the nucleic acid sequence comprises at least 50 consecutive nucleotides.

9. A vector comprising the isolated nucleic acid of claim 8.

10. A host cell comprising the expression vector of claim 9.

11. The host cell of claim 10 which is selected from the group consisting of eukaryotic and prokaryotic cells.

12. The host cell of claim 10 which is selected from the group consisting of bacterial, yeast, insect, mammalian, and plant cells.

13. The isolated nucleic acid of claim 1, wherein the nucleic acid sequence comprises at least 15 consecutive nucleotides.

14. A vector comprising the isolated nucleic acid of claim 13.

15. A host cell comprising the vector of claim 14.

16. The host cell of claim 15 which is selected from the group consisting of eukaryotic and prokaryotic cells.

17. The host cell of claim 15 which is selected from the group consisting of bacterial, yeast, insect, mammalian, and plant cells.

18. An isolated nucleic acid variant which comprises the sequence of SEQ ID NO:6, and contains at least one single nucleotide polymorphism set forth in Table 10.

19. An isolated nucleic acid variant which comprises at least 50 consecutive nucleotides of SEQ ID NO:6, and contains at least one single nucleotide polymorphism set forth in Table 10.

20. An isolated nucleic acid variant which comprises at least 15 consecutive nucleotides of SEQ ID NO:6, and contains at least one single nucleotide polymorphism set forth in Table 10.

21. The isolated nucleic acid variant of claim 20, wherein the single nucleotide polymorphism is selected from the group consisting of T4, T5, T8, T+1, T+2, R1, Q1, Q2, QR+4, QR+6, QR+7, and U−1.

22. The isolated nucleic acid variant of claim 20, wherein the single nucleotide polymorphism selected from the group consisting of D1, F1, I1, L1, R2, T6, T1, T2, T3, and T7.

23. The isolated nucleic acid variant of claim 20 containing at least two single nucleotide polymorphisms selected from the group consisting of:
a. T+2 and QR+4;
b. QR+5 and QR+4;
c. QR+4 and Q+1;
d. QR+6 and Q2; and
e. QR+4 and Q2.

24. The isolated nucleic acid variant of claim 20, wherein the single nucleotide polymorphism is selected from the group consisting of:
a. T5 and T8;
b. T+2 and QR+4;
c. T4 and T5.
d. T+1 and R1 and Q1; and
e. T5 and R1 and Q1.

25. An isolated nucleic acid variant which comprises the sequence of SEQ ID NO:1, and contains at least one single nucleotide polymorphism at a site shown in FIG. 24.

26. An isolated nucleic acid variant which comprises at least 50 consecutive nucleotides of SEQ ID NO:1, and contains at least one single nucleotide polymorphism at a site shown in FIG. 24.

27. An isolated nucleic acid variant which comprises at least 15 consecutive nucleotides of SEQ ID NO:1, and contains at least one single nucleotide polymorphism at a site shown in FIG. 24.

28. An isolated alternate splice variant which comprises at least one exon of SEQ ID NO:1 set forth in FIGS. 9 and 10.

29. An isolated alternate splice variant which comprises at least one exon of SEQ ID NO:1 selected from the group consisting of exons T, R, Q, and U set forth in FIGS. 9 and 10.

30. An isolated alternate splice variant which comprises at least one exon of SEQ ID NO:1 selected from the group consisting of exons A, B, C, D, D′, E, F, G, H, I, J, K, L, L2, M, N, O, P, and S set forth in FIGS. 9 and 10.

31. An isolated alternate splice variant which comprises a sequence selected from the group consisting of SEQ ID NO:2 and SEQ ID NO:350-362.

32. An isolated polypeptide encoded by the nucleic acid of any one of claims 1 and 8.

33. An isolated polypeptide encoded by the nucleic acid of any one of claims 18, 19, 25, and 26.

34. An isolated polypeptide comprising an amino acid sequence selected from the group consisting of:
a. SEQ ID NO:4;
b. an amino acid sequence which is at least 80% identical to SEQ ID NO:4;
c. an amino acid sequence which is at least 75% identical to SEQ ID NO:4; and
d. an amino acid sequence which is at least 65% identical to SEQ ID NO:4.

35. An isolated polypeptide comprising at least 20 consecutive residues of the amino acid sequence of claim 34.

36. An isolated polypeptide comprising at least 7 consecutive residues of the amino acid sequence of claim 34.

37. An antibody or antibody fragment which binds to the isolated polypeptide of claim 32.

38. An antibody or antibody fragment which binds to the isolated polypeptide of claim 33.

39. An antibody or antibody fragment which binds to the isolated polypeptide according to any one of claims 34-36

40. The antibody or antibody fragment of claim 37 which is selected from the group consisting of polyclonal and monoclonal antibodies.

41. The antibody or antibody fragment of claim 38 which is selected from the group consisting of polyclonal and monoclonal antibodies.

42. The antibody or antibody fragment of claim 39 which is selected from the group consisting of polyclonal and monoclonal antibodies.

43. An isolated nucleic acid comprising a nucleotide sequence selected from the group consisting of:
a. SEQ ID NO:6;
b. a nucleotide sequence comprising at least 50 consecutive nucleotides of SEQ ID NO:6; and
c. a nucleotide sequence comprising at least 15 consecutive nucleotides of SEQ ID NO:6.

44. An isolated nucleic acid comprising a nucleotide sequence selected from the group consisting of:
a. SEQ ID NO:364;
b. a nucleotide sequence complementary to SEQ ID NO:364.
c. a nucleotide sequence comprising at least 50 consecutive nucleotides of SEQ ID NO:364;
d. a nucleotide sequence comprising at least 15 consecutive nucleotides of SEQ ID NO:364.
e. SEQ ID NO:365;
f. a nucleotide sequence complementary to SEQ ID NO:365;
g. a nucleotide sequence comprising at least 50 consecutive nucleotides of SEQ ID NO:365; and
h. a nucleotide sequence comprising at least 15 consecutive nucleotides of SEQ ID NO:365.

45. An isolated polypeptide comprising an amino acid sequence selected from the group consisting of:
a. SEQ ID NO:366;
b. an amino acid sequence comprising 20 consecutive residues of SEQ ID NO:366; and
c. an amino acid sequence comprising 7 consecutive residues of SEQ ID NO:M366.

46. An isolated antibody or antibody fragment that binds to the isolated polypeptide of claim 45.

47. The antibody or antibody fragment of claim 46 which is selected from the group consisting of monoclonal and polyclonal antibodies.

48. An isolated antisense nucleic acid comprising at least 15 consecutive nucleotides of a sequence complementary to SEQ ID NO:1.

49. An isolated antisense nucleic acid comprising at least 15 consecutive nucleotides of a sequence complementary to SEQ ID NO:6.

50. A vector comprising the isolated antisense nucleic acid of any one of claims 48-49.

51. A kit for detecting a Gene 216 nucleotide sequence comprising:
a. the isolated nucleic acid of any one of claims 13, 20, and 27; and
b. at least one component to detect binding of the isolated nucleic acid to a Gene 216 nucleotide sequence.

52. A kit for detecting a Gene 216 amino acid sequence comprising:
a. the isolated antibody of claim 42; and
b. at least one component to detect binding of the isolated antibody to a Gene 216 amino acid sequence.

53. A method of identifying a Gene 216 ligand, comprising:
a. contacting the isolated polypeptide of claim 35 with a test agent under conditions that allow the polypeptide to bind to the test agent, and thereby form a complex; and
b. detecting the polypeptide-test agent complex of (a), wherein detection of the complex indicates identification of a Gene 216 ligand.

54. The method of claim 53, wherein the ligand is a metalloprotease inhibitor.

55. The method of claim 54, wherein the metalloprotease inhibitor is a proglutamyl peptide analog.

56. The method of claim 55, wherein the proglutamyl peptide analog is an analog of pyroGlu-Asn-Trp-OH or pyroGlu-Glu-Trp-OH.

57. A pharmaceutical composition comprising the ligand identified according to the method of any one of claims 53-56, and a physiologically acceptable carrier, excipient, or diluent.

58. A pharmaceutical composition comprising the isolated nucleic acid of any one of claims 1, 8, 13, 43, 48, and 49, and a physiologically acceptable carrier, excipient, or diluent.

59. A pharmaceutical composition comprising the vector of any one of claims 4, 9,14, and 48, and a physiologically acceptable carrier, excipient, or diluent.

60. A pharmaceutical composition comprising the isolated antibody or antibody fragment of claim 42, and a physiologically acceptable carrier, excipient, or diluent.

61. A pharmaceutical composition comprising the isolated polypeptide of claim 36 and a physiologically acceptable carrier, excipient, or diluent.

62. A method of identifying a human Gene 216 or ortholog, comprising:
a. contacting the nucleic acid of any one of claims 1, 8, and 13 with a biological sample under conditions that allow the nucleic acid to hybridize to a nucleic acid in the sample, and thereby form a complex; and
b. detecting the hybridization complex of (a), wherein detection of the complex indicates identification of a human Gene 216 or ortholog.

63. A method of treating a chromosome 20 disorder comprising administering the pharmaceutical composition of claim 57 in an amount effective to treat the disorder.

64. The method of claim 63, wherein the chromosome 20 disorder is selected from the group consisting of asthma, obesity, and inflammatory bowel disease.

65. A method of treating a chromosome 20 disorder comprising administering the pharmaceutical composition of claim 58 in an amount effective to treat the disorder.

66. The method of claim 65, wherein the chromosome 20 disorder is selected from the group consisting of asthma, obesity, and inflammatory bowel disease.

67. A method of treating a chromosome 20 disorder comprising administering the pharmaceutical composition of claim 59 in an amount effective to treat the disorder.

68. The method of claim 67, wherein the chromosome 20 disorder is selected from the group consisting of asthma, obesity, and inflammatory bowel disease.

69. A method of treating a chromosome 20 disorder comprising administering the pharmaceutical composition of claim 60 in an amount effective to treat the disorder.

70. The method of claim 69, wherein the chromosome 20 disorder is selected from the group consisting of asthma, obesity, and inflammatory bowel disease.

71. A method of treating a chromosome 20 disorder comprising administering the pharmaceutical composition of claim 61 in an amount effective to treat the disorder.

72. The method of claim 71, wherein the chromosome 20 disorder is selected from the group consisting of asthma, obesity, and inflammatory bowel disease.

73. A transgenic mouse whose genome comprises an introduced null mutation in an endogenous Gene 216.

74. The transgenic mouse of claim 73, wherein both alleles of the endogenous Gene 216 of said mouse have been disrupted.

75. The transgenic mouse of claim 74, wherein the mouse genome further comprises a human Gene 216 nucleic acid sequence.

76. A method of making a homozygous transgenic knockout mouse comprising:
a. disrupting an endogenous Gene 216 in mouse embryonic stem cells;
b. introducing said embryonic stem cells into a mouse blastocyst and transplanting said blastocyst into a pseudopregnant mouse;
c. allowing said blastocyst to develop into a chimeric mouse;
d. breeding said chimeric mouse to produce offspring; and
e. screening said offspring to identify a homozygous transgenic knockout mouse.

77. A method of making a knockout mouse comprising administering the antibody or antibody fragment of claim 47 in an amount effective to disrupt endogenous Gene 216 polypeptide function, thereby making a knockout mouse.

78. A method of forming a crystal of the isolated Gene 216 polypeptide of claim 36 comprising:
a. incubating the polypeptide with a solution selected from the group consisting of the solutions in wells 1-30 in Table 1 under conditions to allow crystalization; and
b. detecting the crystalization in (a), whereby crystalization indicates formation of a Gene 216 polypeptide crystal.

79. A method of diagnosing a chromosome 20 disorder, comprising:
a. contacting the isolated nucleic acid of any one of claims 20-24 with a biological sample under high stringency conditions that allow the nucleic acid to hybridize to a nucleic acid in the sample, and thereby form a complex; and
b. detecting the hybridization complex of (a), wherein detection of the complex indicates diagnosis of a chromosome disorder.

80. The method of claim 79, wherein the disorder is selected from the group consisting of asthma, obesity, and inflammatory bowel disease.

81. A method of diagnosing a chromosome 20 disorder comprising:
a. contacting the isolated antibody or antibody fragment of claim 41 with a biological sample under high stringency conditions that allow the antibody or antibody fragment to bind to an amino acid sequence in the sample, and thereby form a complex; and
b. detecting the complex of (a), wherein detection of the complex indicates diagnosis of a chromosome disorder.

82. A method of determining a pharmacogenetic profile comprising:
a. contacting the isolated nucleic acid of any one of claims 20-24 with a biological sample under high stringency conditions that allow the nucleic acid to hybridize to a nucleic acid in the sample, and thereby form a complex; and
b. detecting the hybridization complex of (a), wherein detection of the complex determines the pharmacogenetic profile.

83. A method of determining a pharmacogenetic profile comprising:
a. contacting the isolated antibody of claim 41 with a biological sample under high stringency conditions that allow the antibody to hybridize to an amino acid sequence in the sample, and thereby form a complex; and
b. detecting the complex of (a), wherein detection of the complex determines the pharmacogenetic profile.

84. A cell line comprising the isolated nucleic acid of any one of claims 8, 19, 26, and 28.

85. A biochip comprising the isolated nucleic acid of any one of claims 8, 19, 26, and 28.
